(19) United States
(12) Patent Application Publication
(10) Pub. No.: US 2025/0375537 A1
(43) Pub. Date: Dec. 11, 2025

(54) Systems and Methods for Genomic Editing

(71) Applicant: Intellia Therapeutics, Inc., Cambridge, MA (US)

(72) Inventors: Sabin Mulepati, Somerville, MA (US); Christian Dombrowski, Auburndale, MA (US); Jennifer Lauren Curran, Cambridge, MA (US)

(73) Assignee: Intellia Therapeutics, Inc., Cambridge, MA (US)

(21) Appl. No.: 19/241,998

(22) Filed: Jun. 18, 2025

Related U.S. Application Data
(63) Continuation of application No. PCT/US2023/085661, filed on Dec. 22, 2023.
(60) Provisional application No. 63/477,093, filed on Dec. 23, 2022.

(51) Int. Cl. A61K 48/00; C12N 9/22; C12N 15/11; C12N 15/88; C12N 15/90

(52) U.S. Cl. CPC A61K 48/0058; A61K 48/0041; C12N 9/226; C12N 15/11; C12N 15/88; C12N 15/907; C12N 2310/20

(57) ABSTRACT

Methods and compositions for genetically modifying a cell are provided.

### CROSS-REFERENCE TO RELATED APPLICATION

This application is a continuation of International Application No. PCT/US2023/085661, filed Dec. 22, 2023, which claims the benefit of priority to U.S. Provisional Application No. 63/477,093, filed Dec. 23, 2022, both of which are herein incorporated by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 12, 2024, is named “01155-0056-00US.xml” and is 1,645,487 bytes in size. The sequence listing contained in this .XML file is part of the specification and is hereby incorporated by reference herein in its entirety.

### INTRODUCTION AND SUMMARY

The present disclosure relates to methods, compositions, and systems for genomic editing.

The ability to introduce precise and reproducible deletions into the genome of a cell is of interest for gene editing and clinical therapeutic applications. For example, adoptive cell therapy approaches using genetically modified immune cells have become an attractive modality to treat a variety of conditions and diseases, including cancers, to reconstitute cell lineages and immune system defense. However, the clinical application of cell product therapies has been challenging in part due to the complex genetic engineering requirements. The ability to precisely excise genetic elements, including start codons, splice sites, and transcription factor binding sites, while minimizing the risk of off-target cleavage and locus inversions, is thus of great interest to the field of genetic engineering.

CRISPR/Cas9 genome editing has been demonstrated to be highly efficient; however, it has been challenging to generate precise deletions. Double strand breaks (DSBs) may be repaired via the error-prone non-homologous end-joining (NHEJ) pathway, generating small insertions or deletions around a break site. While this process may therefore generate a deletion around the site of a DSB, the size of this deletion may vary considerably on a cell-to-cell and even allele-to-allele basis. Thus, there is a need for a more efficient approach for generating precise and reproducible deletions within the genome of a cell.

The methods provided herein comprise using an orthogonal Cas9-Cas9 fusion system for precise genome editing applications, providing substantial advantages over traditional methods.

Accordingly, the present disclosure provides a method of producing a modification in the genome of a target cell, the method comprising contacting the cell with: (a) a fusion protein, or a nucleic acid encoding a fusion protein, wherein the fusion protein comprises a first cleavase and a second cleavase, wherein the first cleavase is a Streptococcus pyogenes (Spy)Cas9 cleavase, said SpyCas9 cleavase comprising a R1333K mutation within its protospacer adjacent motif recognition domain; and the second cleavase is a Neisseria meningitidis (Nme)Cas9 cleavase, a Campylobacter jejuni (Cje) Cas9 cleavase, or a Simonsiella muelleri (Smu) Cas9 cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, a method of producing a cell or a population of cells comprising a modification in the genome of the target cell or cells is provided. In some embodiments, the method comprises contacting the cell with: (a) a fusion protein, or a nucleic acid encoding a fusion protein, wherein the fusion protein comprises a first cleavase and a second cleavase, wherein the first cleavase is a SpyCas9 cleavase, said SpyCas9 cleavase comprising a R1333K mutation within its protospacer adjacent motif recognition domain; and the second cleavase is a NmeCas9 cleavase, a Cje Cas9 cleavase, or a Smu Cas9 cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, a polynucleotide is provided, comprising an open reading frame (ORF) encoding a fusion protein, wherein the fusion protein comprises a first cleavase and a second cleavase, wherein: the first cleavase is a S. pyogenes (Spy)Cas9 cleavase, said SpyCas9 cleavase comprising a R1333K mutation within its protospacer adjacent motif recognition domain; and the second cleavase is a NmeCas9 cleavase, a Cje Cas9 cleavase, or a Smu Cas9 cleavase.

In some embodiments, a composition is provided.

In some embodiments, the composition comprises (a) a polynucleotide comprising an open reading frame (ORF) encoding a fusion protein, wherein the fusion protein comprises a first cleavase and a second cleavase, wherein: the first cleavase is a S. pyogenes (Spy)Cas9 cleavase, said SpyCas9 cleavase comprising a R1333K mutation within its protospacer adjacent motif recognition domain; and the second cleavase is a NmeCas9 cleavase, a Cje Cas9 cleavase, or a Smu Cas9 cleavase; and (b) a first guide RNA that directs the first cleavase to a first genomic locus; and (c) a second guide RNA that directs the second cleavase to a second genomic locus, wherein the second genomic locus is different from the first genomic locus.

The following is a non-exhaustive listing of embodiments provided herein.

Embodiment 1 is a method of producing a modification in the genome of a target cell, the method comprising contacting the cell with:
(a) a fusion protein, or a nucleic acid encoding a fusion protein, wherein the fusion protein comprises a first cleavase and a second cleavase, wherein:
   a. the first cleavase is a Streptococcus pyogenes (Spy)Cas9 cleavase, said SpyCas9 cleavase comprising a R1333K mutation within its protospacer adjacent motif recognition domain; and
   b. the second cleavase is a Neisseria meningitidis (Nme)Cas9 cleavase, a Campylobacter jejuni (Cje) Cas9 cleavase, or a Simonsiella muelleri (Smu) Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

Embodiment 2 is a method of producing a cell or a population of cells comprising a modification in the genome of the target cell or cells, the method comprising contacting the cell or cells with:
(a) a fusion protein, or a nucleic acid encoding a fusion protein, wherein the fusion protein comprises a first cleavase and a second cleavase, wherein:
   a. the first cleavase is a S. pyogenes (Spy)Cas9 cleavase, said SpyCas9 cleavase comprising a R1333K mutation within its protospacer adjacent motif recognition domain; and
   b. the second cleavase is a N. meningitidis (Nme)Cas9 cleavase, a C. jejuni (Cje)Cas9 cleavase, or a S. muelleri (Smu)Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

Embodiment 3 is the method of any one of Embodiment 1 or 2, wherein the first cleavase is located N-terminal to the second cleavase.

Embodiment 4 is the method of any one of Embodiment 1 or 2, wherein the first cleavase is located C-terminal to the second cleavase.

Embodiment 5 is the method of any one of the preceding Embodiments, wherein (i) the SpyCas9 cleavase comprises an amino acid sequence of SEQ ID NO: 105 or an amino acid sequence that is at least 85%, at least 90%, or at least 95% identical to SEQ ID NO: 105; or (ii) the nucleotide encoding the SpyCas9 cleavase comprises an open reading frame (ORF) comprising a sequence of SEQ ID NO: 104 or a nucleotide sequence that is at least 85, at least 90%, or at least 95% identical to SEQ ID NO: 104.

Embodiment 6 is the method of any one of the preceding Embodiments, wherein the second cleavase is a NmeCas9 cleavase.

Embodiment 7 is the method of any one of the preceding Embodiments, wherein the NmeCas9 cleavase is an Nme1Cas9, an Nme2Cas9, or an Nme3Cas9.

Embodiment 8 is the method of any one of the preceding Embodiments, wherein (i) the NmeCas9 cleavase comprises an amino acid sequence of any one of SEQ ID NO: 22, 107, 109, 120, 127, 136, or 137 or an amino acid sequence that is at least 85%, at least 90%, or at least 95% identical to any one of SEQ ID NO: 22, 107, 109, 120, 127, 136, or 137; or (ii) the nucleotide encoding the NmeCas9 cleavase comprises a nucleotide sequence of any one of SEQ ID NO: 21, 106, 108, 121-126, 128-133, 134, 135, 138, or 139; or a nucleotide sequence that is at least 85%, at least 90%, or at least 95% identical to any one of SEQ ID NO: SEQ ID NO: 21, 106, 108, 121-126, 128-133, 134, 135, 138, or 139.

Embodiment 9 is the method of any one of the preceding Embodiments, wherein (a) the NmeCas9 cleavase is a Nme2Cas9 comprises an amino acid sequence of any one of SEQ ID NO: 22, 109, or 136 or an amino acid sequence that is at least 85%, at least 90%, at least 95% identical to any one of SEQ ID NO: 22, 109, or 136; or (b) the nucleotide encoding the NmeCas9 cleavase comprises a nucleotide sequence of any one of SEQ ID NO: 21, 108, or 138; or a nucleotide sequence that is at least 85%, at least 90%, or at least 95% identical to any one of SEQ ID NO: SEQ ID NO: 21, 108, or 138.

Embodiment 10 is the method of any one of the preceding Embodiments, wherein (a) the CjeCas9 cleavase comprises an amino acid sequence of SEQ ID NO: 144; or an amino acid sequence that is at least 85%, at least 90%, or at least 95% identical to SEQ ID NO: 144; or (b) the nucleotide encoding the CjeCas9 cleavase comprises a sequence of SEQ ID NO: 143 or a nucleotide sequence that is at least 85%, at least 90%, or at least 95% identical to SEQ ID NO: 143.

Embodiment 11 is the method of any one of the preceding Embodiments, wherein (a) the SmuCas9 cleavase comprises an amino acid sequence of SEQ ID NO: 142; or an amino acid sequence that is at least 85%, at least 90%, or at least 95% identical to SEQ ID NO: 142; or (b) the nucleotide encoding the SmuCas9 cleavase comprises an open reading frame (ORF) comprising a sequence of SEQ ID NO: 140 or 141 or a nucleotide sequence that is at least 85%, at least 90%, or at least 95% identical to SEQ ID NO: 140 or 141.

Embodiment 12 is the method of any one of the preceding Embodiments, wherein the fusion protein comprises a peptide linker between the first cleavase and the second cleavase.

Embodiment 13 is the method of any one of the preceding Embodiments, wherein the fusion protein comprises a peptide linker between the first cleavase and the second cleavase and the peptide linker comprises at least 10, at least 20, at least 30, at least 40, at least 50, at least 60, at least 70, at least 80 amino acids.

Embodiment 14 is the method of any one of the preceding Embodiments, wherein the fusion protein comprises a peptide linker between the first cleavase and the second cleavase and the peptide linker comprises 11, 21, 31, 41, 51, 61, 71, or 81 amino acid residues.

Embodiment 15 is the method of any one of the preceding Embodiments, wherein the fusion protein comprises a peptide linker between the first cleavase and the second cleavase and the peptide linker comprises an amino acid sequence of any one of SEQ ID NOs: 150-158; or an amino acid sequence is at least 85%, at least 90%, or at least 95% identical to any one of SEQ ID NOs: 150-158.

Embodiment 16 is the method of any one of the preceding Embodiments, wherein the fusion protein comprises a nuclear localization signal (NLS).

Embodiment 17 is the method of any one of the preceding Embodiments, wherein the fusion protein comprises a nuclear localization signal (NLS) and the NLS is present at the C-terminus of the fusion protein.

Embodiment 18 is the method of any one of the preceding Embodiments, wherein the fusion protein comprises a nuclear localization signal (NLS) and the NLS is present at the N-terminus of the fusion protein.

Embodiment 19 is the method of any one of the preceding Embodiments, wherein the fusion protein comprises a nuclear localization signal (NLS) and the NLS is present at both the N-terminus and C-terminus of the fusion protein.

Embodiment 20 is the method of any one of the preceding Embodiments, wherein the fusion protein comprises a nuclear localization signal (NLS), and wherein the NLS comprises a sequence having at least 80%, at least 85%, at least 90%, or at least 95% identity to any one of SEQ ID NOs: 366-369 and 371-384.

Embodiment 21 is the method of any one of the preceding Embodiments, wherein the fusion protein comprises a nuclear localization signal (NLS), and wherein the NLS is encoded by a sequence having at least 80%, at least 85%, at least 90%, at least 95%, at least 98% or 100% identity to the sequence of any one of SEQ ID NOs: 370 and 385-397.

Embodiment 22 is the method of any one of the preceding Embodiments, wherein the fusion protein comprises one, two, or three nuclear localization signals (NLSs) independently selected from SEQ ID NOs: 366-369 and 371-384.

Embodiment 23 is the method of any one of the preceding Embodiments, wherein the fusion protein comprises, from N-terminus to C-terminus:
a. the first cleavase;
b. a peptide linker, optionally wherein the linker comprises 81 amino acid residues;
c. the second cleavase; and
d. an NLS comprising an SV40 NLS.

Embodiment 24 is the method of any one of Embodiments 1-22, wherein the fusion protein comprises, from N-terminus to C-terminus:
a. a first NLS, wherein the first NLS comprises an SV40 NLS;
b. the second cleavase;
c. a peptide linker, optionally wherein the peptide linker comprises 41 amino acids;
d. the first cleavase;
e. a second NLS, wherein the second NLS comprising an SV40 NLS.

Embodiment 25 is the method of any one of Embodiments 1-22, wherein the fusion protein comprises, from N-terminus to C-terminus:
a. the second cleavase;
b. a peptide linker, optionally wherein the peptide linker comprises 41 amino acids;
c. the first cleavase; and
d. an NLS, optionally wherein the NLS comprises an SV40 NLS.

Embodiment 26 is the method of any one of the preceding Embodiments, wherein
(a) the fusion protein comprise an amino acid sequence of SEQ ID NOs: 3, 5, 7, 10, 13, 16, 40, 43, 45, 47, 50, 52, 55, 57, 60, 62, 65, 67, 70, 72, 75, 77, 80, 82, 85, 87, 90, 92, 101, or 105, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, 13, 16, 40, 43, 45, 47, 50, 52, 55, 57, 60, 62, 65, 67, 70, 72, 75, 77, 80, 82, 85, 87, 90, 92, 101, or 105; or
(b) the nucleic acid encoding the fusion protein comprises a nucleotide sequence of SEQ ID NOs: 1-2, 4, 6, 8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104.

Embodiment 27 is the method of any one of the preceding Embodiments, wherein
(a) the fusion protein comprise an amino acid sequence of SEQ ID NOs: 3, 5, 7, 10, or 13, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, or 13; or
(b) the nucleic acid encoding the fusion protein comprises a nucleotide sequence of SEQ ID NOs: 1, 2, 4, 6, 8, 9, 11 or 12, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11 or 12.

Embodiment 28 is the method of any one of the preceding Embodiments, wherein
(a) the fusion protein comprise an amino acid sequence of SEQ ID NOs: 5 or 9 or n amino acid sequence that is at least 85%, at least 90%, or at least 95%% identical to an amino acid sequence of SEQ ID NOs: 5 or 99; or
(b) the nucleic acid encoding the fusion protein comprises a nucleotide sequence of SEQ ID NOs: 4, 6, 7, or 8, a nucleotide sequence that is at least 85%, at least 90%, or at least 95% identical to SEQ ID NO: 4, 6, 7 or 8,

Embodiment 29 is a method of producing a modification in the genome of a target cell, the method comprising:
(a) contacting the cell with a first polypeptide, or a nucleic acid encoding the first polypeptide, wherein the first polypeptide comprises a first cleavase and a first intein, wherein the first cleavase is a S. pyogenes (Spy)Cas9 cleavase, said SpyCas9 cleavase comprising a R1333K mutation within its protospacer adjacent motif recognition domain;
(b) contacting the cell with a second polypeptide, or a nucleic acid encoding the first polypeptide, wherein the second polypeptide comprises a second cleavase and a second intein capable of binding the first intein, wherein the second cleavase is a N. meningitidis (Nme)Cas9 cleavase, a C. jejuni (Cje) Cas9 cleavase, or a S. muelleri (Smu) Cas9 cleavase;
and wherein the first polypeptide binds to the second polypeptide through intein catalysis,
(c) contacting the cell with a first guide RNA that directs the first cleavase to a first genomic locus; and
(d) contacting the cell with a second guide RNA that directs the second cleavase to a second genomic locus, wherein the second genomic locus is different from the first genomic locus.

Embodiment 30 is the method of Embodiment 29, wherein the first polypeptide comprises, from N-terminus to C-terminus:
a. the first intein;
b. the first cleavase, wherein the first cleavase is a S. pyogenes (Spy)Cas9 cleavase, said SpyCas9 cleavase comprising a R1333K mutation within its protospacer adjacent motif recognition domain; and
c. a first NLS comprising an SV40 NLS.

Embodiment 31 is the method of Embodiment 29 or 30, wherein the second polypeptide comprises, from N-terminus to C-terminus:
a. a second NLS comprising an SV40 NLS;
b. a third NLS comprising a nucleoplasmin NLS;
c. the second cleavase;
d. a peptide linker, optionally wherein the peptide linker comprises 41 or 81 amino acid residues; and
e. the second intein capable of binding the first intein.

Embodiment 32 is the method of any one of Embodiments 29-31, wherein the first polypeptide comprise an amino acid sequence of SEQ ID NOs: 28 or 31 or an amino acid sequence that is at least 85%, at least 90%, or at least 95% identical to SEQ ID NO: 28 or 31; or the nucleic acid or nucleic acids encoding the polypeptide or polypeptides comprises a sequence of SEQ ID NOs: 27 or 30, or a nucleotide sequence that is at least 85%, at least 90%, or at least 95% identical to SEQ ID NOs: 27 or 30.

Embodiment 33 is the method of any one of Embodiments 29-33, wherein the second polypeptide comprise an amino acid sequence of SEQ ID NOs: 25 or 34 or an amino acid sequence that is at least 85%, at least 90%, or at least 95% identical to SEQ ID NO: 25 or 34; or the nucleic acid or nucleic acids encoding the polypeptide or polypeptides comprises a sequence of SEQ ID NOs: 24 or 33, or a nucleotide sequence that is at least 85%, at least 90%, or at least 95% identical to SEQ ID NOs: 24 or 33.

Embodiment 34 is the method of any one of the preceding Embodiments, wherein the first guide RNA and the second guide RNA target two non-overlapping genomic loci.

Embodiment 35 is The method of the immediately preceding Embodiment, wherein the two non-overlapping genomic loci are separated by equal to or less than 150, 140, 130, 120, 110, 100, 90, 80, 70, 60, 50, 40, 30, or 25 nucleotides.

Embodiment 36 is the method of the immediately preceding Embodiment, wherein the two non-overlapping genomic loci are separated by equal to or less than 110 nucleotides.

Embodiment 37 is the method of any one of the preceding Embodiments, wherein the first guide RNA is a single guide RNA (sgRNA).

Embodiment 38 is the method of any one of the preceding Embodiments, wherein the first guide RNA is a SpyCas9 guide RNA.

Embodiment 39 is the method of the immediately preceding Embodiment, wherein the SpyCas9 guide RNA is a single guide RNA comprising:
a conserved portion of an sgRNA comprising an upper stem and hairpin region, wherein every nucleotide in the upper stem region is modified with 2′-O-Me, and every nucleotide in the hairpin region is modified with 2′-O-Me;
a 3′ end modification comprising 2′-O-Me modified nucleotides at the last three nucleotides of the 3′ end and phosphorothioate (PS) bonds between the last four nucleotides of the 3′ end; and
5′ end modification comprising 2′-O-Me modified nucleotides at the first three nucleotides of the 5′ end; and phosphorothioate (PS) bonds between the first four nucleotides of the 5′ end.

Embodiment 40 is the method of Embodiment 38 or 39, wherein the SpyCas9 guide RNA is a short-single guide RNA (short-sgRNA) comprising a conserved portion of an sgRNA comprising a hairpin region, wherein the hairpin region lacks at least 5-10 nucleotides and wherein the short-sgRNA comprises (i) a 5′ end modification or (ii) a 3′ end modification.

Embodiment 41 is the method of any one of the preceding Embodiments, wherein the first guide RNA is a SpyCas9 guide RNA that is a single guide RNA comprising a nucleotide sequence selected from SEQ ID NOs: 159-167, 170-177, and 180-194, or a nucleotide sequence that is at least 85%, 90%, or 95% identical to SEQ ID NOs: 159-167, 170-177, and 180-194.

Embodiment 42 is the method of any one of the preceding Embodiments, wherein the second guide RNA is a single guide RNA (sgRNA).

Embodiment 43 is the method of the immediately preceding Embodiment, wherein the second guide RNA is a shortened or chemically modified single guide RNA (sgRNA).

Embodiment 44 is the method of any one of the preceding Embodiments, wherein the second guide RNA is a NmeCas9 guide RNA.

Embodiment 45 is the method of any one of the preceding Embodiments, wherein the second guide RNA is a NmeCas9 guide RNA that is a single guide RNA comprising a nucleotide sequence selected from SEQ ID NOs: 280-297, or a nucleotide sequence that is at least 85%, 90%, or 95% identical to SEQ ID NOs: 280-297.

Embodiment 46 is the method of the immediately preceding Embodiment, wherein the second guide RNA comprises one or more internal polyethylene glycol (PEG) linker, optionally wherein the second guide RNA comprises at least 85%, 90%, 95%, 99%, 100% identical to a sequence selected from SEQ ID NOs: 272-278.

Embodiment 47 is the method of any one of the preceding Embodiments, wherein one or both of the guide RNAs comprises one or more mismatches to the target sequences.

Embodiment 48 is the method of any one of the preceding Embodiments, wherein the nucleic acid encoding the fusion protein is delivered to the cell on at least one vector.

Embodiment 49 is the method of any one of the preceding Embodiments, wherein the fusion protein or the nucleic acid encoding the fusion protein are delivered to the cell via electroporation.

Embodiment 50 is the method of any one of the preceding Embodiments, wherein the first guide RNA is delivered to the cell via electroporation.

Embodiment 51 is the method of any one of the preceding Embodiments, wherein the second guide RNA is delivered to the cell via electroporation.

Embodiment 52 is the method of any one of Embodiments 1-48, wherein one or more of the nucleic acids encoding the fusion protein, the first guide RNA, and the second guide RNA are associated with one or more lipid nanoparticle (LNP).

Embodiment 53 is the method of any one of Embodiments 1-48 or 52, wherein the nucleic acids encoding the fusion protein are each associated with a separate lipid nanoparticle (LNP).

Embodiment 54 is the method of any one of Embodiments 1-48 or 52-53, wherein the first guide RNA and the second guide RNA are associated with a same lipid nanoparticle (LNP).

Embodiment 55 is the method of any one of Embodiments 1-48 or 52-54, wherein all of the nucleic acids encoding the fusion protein, the first guide RNA, and the second guide RNA are associated with a same lipid nanoparticle (LNP).

Embodiment 56 is the method of any one of Embodiments 1-48 or 52-55, wherein the LNP comprises (i) an ionizable lipid; (ii) a helper lipid; (iii) a stealth lipid; (iv) a neutral lipid; or combinations of one or more of (i)-(iv).

Embodiment 57 is the method of the immediately preceding Embodiment, wherein the ionizable lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 58 is the method of Embodiment 56 or 57, wherein the helper lipid is cholesterol.

Embodiment 59 is the method of any one of Embodiments 56-58 immediately preceding Embodiments, wherein the stealth lipid is PEG-DMG.

Embodiment 60 is the method of any one of Embodiments 56-59, wherein the PEG-DMG is 1,2-dimyristoyl-rac-glycero-3-methoxypolyethylene glycol-2000 (PEG2k-DMG).

Embodiment 61 is the method of any one of Embodiments 56-60, wherein the neutral lipid is DSPC.

Embodiment 62 is the method of any one of Embodiments 56-61, wherein the LNP composition comprises about 50 mol-% ionizable lipid; about 9 mol-% neutral lipid; about 3 mol-% of stealth lipid, and the remainder of the lipid component is helper lipid such as cholesterol.

Embodiment 63 is the method of any one of Embodiments 56-62, wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

Embodiment 64 is the method of any one of the preceding Embodiments, wherein the modification is in vivo.

Embodiment 65 is the method of any one of Embodiments 1-63, wherein the modification is ex vivo.

Embodiment 66 is the method of any one of the preceding Embodiments, wherein the modification comprises a deletion of equal to or less than 150, 140, 130, 120, 110, 100, 90, 80, 70, 60, 50, 40, 30 nucleotides.

Embodiment 67 is the method of the immediately preceding Embodiment, wherein the modification comprises a deletion of equal to or less than 150, 140, 130, 120, 110, 100, 90, 80, 70, 60, 50, 40, 30, or 25 contiguous nucleotides.

Embodiment 68 is the method of any one of the preceding Embodiments, wherein the modification comprises a deletion of equal to or larger than 25, 35, 45, 55, 65, 75, 85, 95, 100, 105 nucleotides.

Embodiment 69 is the method of the immediately preceding Embodiment, wherein the modification comprises a deletion of equal to or larger than 25, 35, 45, 55, 65, 75, 85, 95, 100, 105 contiguous nucleotides.

Embodiment 70 is the method of any one of the preceding Embodiments, wherein the modification comprises a deletion of each of the nucleotides between a first cleavage site and a second cleavage site.

Embodiment 71 is the method of any one of Embodiments 66-70, wherein the deletion comprises one or both protospacer adjacent motif (PAM) sites recognized by the first cleavase or the second cleavase.

Embodiment 72 is the method of any one of the preceding Embodiments, wherein the modification increases the expression of one or more RNAs or proteins.

Embodiment 73 is the method of any one of the preceding Embodiments, wherein the modification increases the expression of one or more mRNAs by at least two-fold.

Embodiment 74 is the method of any one of the preceding Embodiments, wherein the modification increases the expression of one or more proteins by at least two-fold.

Embodiment 75 is the method of any one of the preceding Embodiments, wherein the modification results in the deletion of a start codon.

Embodiment 76 is the method of any one of the preceding Embodiments, wherein the modification reduces or eliminates the expression of one or more mRNAs or proteins.

Embodiment 77 is the method of any one of the preceding Embodiments, wherein the modification reduces or eliminates the expression of one or more mRNAs by at least 50%, at least 55%, at least 60%, at least 65%, at least 70%, at least 75%, at least 80%, at least 85%, at least 90%, or at least 95%.

Embodiment 78 is the method of any one of the preceding Embodiments, wherein the modification reduces or eliminates the expression of one or more proteins by at least 50%, at least 55%, at least 60%, at least 65%, at least 70%, at least 75%, at least 80%, at least 85%, at least 90%, or at least 95%.

Embodiment 79 is the method of any one of the preceding Embodiments, wherein the cell is in a subject.

Embodiment 80 is the method of any one of the preceding Embodiments, wherein the cell is a kidney cell.

Embodiment 81 is the method of any one of the preceding Embodiments, wherein the cell is a liver cell.

Embodiment 82 is the method of any one of the preceding Embodiments, wherein the cell is selected from: a mesenchymal stem cell; a hematopoietic stem cell (HSC); a mononuclear cell; an endothelial progenitor cells (EPC); a neural stem cells (NSC); a limbal stem cell (LSC); a tissue-specific primary cell or a cell derived therefrom (TSC), an induced pluripotent stem cell (iPSC); an ocular stem cell; a pluripotent stem cell (PSC); an embryonic stem cell (ESC); and a cell for organ or tissue transplantation.

Embodiment 83 is the method of any one of the preceding Embodiments, wherein the cell is an immune cell.

Embodiment 84 is the method of any one of the preceding Embodiments, wherein the cell is a lymphocyte.

Embodiment 85 is the method of any one of the preceding Embodiments, wherein the cell is a T-cell.

Embodiment 86 is an engineered cell or population of engineered cells altered by the method of any one of the preceding Embodiments.

Embodiment 87 is the engineered cell or population of engineered cells of the immediately preceding Embodiment, wherein the genetic modification comprises a deletion of equal to or less than 110, 100, 90, 80, 70, 60, 50, 40, 30, or 25 nucleotides.

Embodiment 88 is the engineered cell or population of engineered cells of any one of Embodiments 86 or 87, wherein the deletion comprises one or both protospacer adjacent motif (PAM) sites.

Embodiment 89 is a polynucleotide comprising an open reading frame (ORF) encoding a fusion protein, wherein the fusion protein comprises a first cleavase and a second cleavase, wherein:
a. the first cleavase is a S. pyogenes (Spy)Cas9 cleavase, said SpyCas9 cleavase comprising a R1333K mutation within its protospacer adjacent motif recognition domain; and
b. the second cleavase is a N. meningitidis (Nme)Cas9 cleavase, a C. jejuni (Cje) Cas9 cleavase, or a S. muelleri (Smu) Cas9 cleavase.

Embodiment 90 is a composition comprising
(a) a polynucleotide comprising an open reading frame (ORF) encoding a fusion protein, wherein the fusion protein comprises a first cleavase and a second cleavase, wherein:
   a. the first cleavase is a S. pyogenes (Spy)Cas9 cleavase, said SpyCas9 cleavase comprising a R1333K mutation within its protospacer adjacent motif recognition domain; and
   b. the second cleavase is a N. meningitidis (Nme)Cas9 cleavase, a C. jejuni (Cje) Cas9 cleavase, or a Simonsiella muelleri (Smu) Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

Embodiment 91 is a composition comprising
(a) a first polynucleotide comprising an ORF encoding the first polypeptide, wherein the first polypeptide comprises first cleavase and a first intein, wherein the first cleavase is a S. pyogenes (Spy)Cas9 cleavase, said SpyCas9 cleavase comprising a R1333K mutation within its protospacer adjacent motif recognition domain;
(b) a second polynucleotide comprising an ORF encoding the second polypeptide, wherein the second polypeptide comprises a second cleavase and a second intein capable of binding the first intein, wherein the second cleavase is a N. meningitidis (Nme)Cas9 cleavase, a C. jejuni (Cje) Cas9 cleavase, or a Simonsiella muelleri (Smu) Cas9 cleavase;
   and wherein the first polypeptide binds to the second polypeptide through intein catalysis,

Embodiment 92 is one or more lipid nanoparticles comprising:
(a) a polynucleotide comprising an open reading frame (ORF) encoding a fusion protein, wherein the fusion protein comprises a first cleavase and a second cleavase, wherein:
   a. the first cleavase is a S. pyogenes (Spy)Cas9 cleavase, said SpyCas9 cleavase comprising a R1333K mutation within its protospacer adjacent motif recognition domain; and
   b. the second cleavase is a N. meningitidis (Nme)Cas9 cleavase, a C. jejuni (Cje) Cas9 cleavase, or a Simonsiella muelleri (Smu) Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

Embodiment 93 is the polynucleotide, composition or lipid nanoparticles of any one of Embodiments 89-92, wherein (i) the SpyCas9 cleavase comprises an amino acid sequence of SEQ ID NO: 105 or an amino acid sequence that is at least 85%, at least 90%, or at least 95% identical to SEQ ID NO: 105; or (ii) the nucleotide encoding the SpyCas9 cleavase comprises an open reading frame (ORF) comprising a sequence of SEQ ID NO: 104 or a nucleotide sequence that is at least 85, at least 90%, or at least 95% identical to SEQ ID NO: 104.

Embodiment 94 is the polynucleotide, composition or lipid nanoparticles of any one of Embodiments 89-93, wherein the second cleavase is a NmeCas9 cleavase.

Embodiment 95 is the polynucleotide, composition or lipid nanoparticles of any one of Embodiments 89-94, wherein the NmeCas9 cleavase is an Nme1Cas9, an Nme2Cas9, or an Nme3Cas9.

Embodiment 96 is the polynucleotide, composition or lipid nanoparticles of any one of Embodiments 89-95, wherein (i) the NmeCas9 cleavase comprises an amino acid sequence of any one of SEQ ID NO: 22, 107, 109, 120, 127, 136, or 137 or an amino acid sequence that is at least 85%, at least 90%, at least 95% identical to any one of SEQ ID NO: 22, 107, 109, 120, 127, 136, or 137; or (ii) the nucleotide encoding the NmeCas9 cleavase comprises a nucleotide sequence of any one of SEQ ID NO: 21, 106, 108, 121-126, 128-133, 134, 135, 138, or 139; or a nucleotide sequence that is at least 85%, at least 90%, or at least 95% identical to any one of SEQ ID NO: SEQ ID NO: 21, 106, 108, 121-126, 128-133, 134, 135, 138, or 139.

Embodiment 97 is the polynucleotide, composition or lipid nanoparticles of any one of Embodiments 89-96, wherein (a) the NmeCas9 cleavase is a Nme2Cas9 comprises an amino acid sequence of any one of SEQ ID NO: 22, 109, or 136, or an amino acid sequence that is at least 85%, at least 90%, at least 95% identical to any one of SEQ ID NO: 22, 109, or 136; or (b) the nucleotide encoding the NmeCas9 cleavase comprises a nucleotide sequence of any one of SEQ ID NO: 21, 108, or 138; or a nucleotide sequence that is at least 85%, at least 90%, or at least 95% identical to any one of SEQ ID NO: SEQ ID NO: 21, 108, or 138.

Embodiment 98 is the polynucleotide, composition or lipid nanoparticles of any one of Embodiments 89-97, wherein (a) the CjeCas9 cleavase comprises an amino acid sequence of SEQ ID NO: 144; or an amino acid sequence that is at least 85%, at least 90%, or at least 95% identical to SEQ ID NO: 144; or (b) the nucleotide encoding the CjeCas9 cleavase comprises a sequence of SEQ ID NO: 143 or a nucleotide sequence that is at least 85%, at least 90%, or at least 95% identical to SEQ ID NO: 143.

Embodiment 99 is the polynucleotide, composition or lipid nanoparticles of any one of Embodiments 89-98, wherein (a) the SmuCas9 cleavase comprises an amino acid sequence of SEQ ID NO: 142; or an amino acid sequence that is at least 85%, at least 90%, or at least 95% identical to SEQ ID NO: 142; or (b) the nucleotide encoding the SmuCas9 cleavase comprises an open reading frame (ORF) comprising a sequence of SEQ ID NO: 140 or 141 or a nucleotide sequence that is at least 85%, at least 90%, or at least 95% identical to SEQ ID NO: 140 or 141.

Embodiment 100 is the polynucleotide, composition or lipid nanoparticles of any one of Embodiments 89-99, wherein the fusion protein comprises a peptide linker between the first cleavase and the second cleavase.

Embodiment 101 is the polynucleotide, composition or lipid nanoparticles of any one of Embodiments 89-100, wherein the fusion protein comprises a peptide linker between the first cleavase and the second cleavase and the peptide linker comprises at least 10, at least 20, at least 30, at least 40, at least 50, at least 60, at least 70, at least 80 amino acids.

Embodiment 102 is the polynucleotide, composition or lipid nanoparticles of any one of Embodiments 89-101, wherein the fusion protein comprises a peptide linker between the first cleavase and the second cleavase and the peptide linker comprises 11, 21, 31, 41, 51, 61, 71, or 81 amino acid residues.

Embodiment 103 is the polynucleotide, composition or lipid nanoparticles of any one of Embodiments 89-102, wherein the fusion protein comprises a peptide linker between the first cleavase and the second cleavase and the peptide linker comprises an amino acid sequence of any one of SEQ ID NOs: 150-158; or an amino acid sequence is at least 85%, at least 90%, or at least 95% identical to any one of SEQ ID NOs: 150-158.

Embodiment 104 is the polynucleotide, composition or lipid nanoparticles of any one of Embodiments 89-103, wherein the fusion protein comprises a nuclear localization signal (NLS).

Embodiment 105 is the polynucleotide, composition or lipid nanoparticles of any one of Embodiments 89-104, wherein the fusion protein comprises a nuclear localization signal (NLS) and the NLS is present at the C-terminus of the fusion protein.

Embodiment 106 is the polynucleotide, composition or lipid nanoparticles of any one of Embodiments 89-105, wherein the fusion protein comprises a nuclear localization signal (NLS) and the NLS is present at the N-terminus of the fusion protein.

Embodiment 107 is the polynucleotide, composition or lipid nanoparticles of any one of Embodiments 89-106, wherein the fusion protein comprises a nuclear localization signal (NLS) and the NLS is present at both the N-terminus and C-terminus of the fusion protein.

Embodiment 108 is the polynucleotide, composition or lipid nanoparticles of any one of any one of Embodiments 89-107, wherein the fusion protein comprises a nuclear localization signal (NLS), and wherein the NLS comprises a sequence having at least 80%, at least 85%, at least 90%, or at least 95% identity to any one of SEQ ID NOs: 366-369 and 371-384.

Embodiment 109 is the polynucleotide, composition or lipid nanoparticles of any one of Embodiments 89-108, wherein the fusion protein comprises a nuclear localization signal (NLS), and wherein the NLS is encoded by a sequence having at least 80%, at least 85%, at least 90%, at least 95%, at least 98% or 100% identity to the sequence of any one of SEQ ID NOs: 370 and 385-397.

Embodiment 110 is the polynucleotide, composition or lipid nanoparticles of any one of Embodiments 89-109, wherein the fusion protein comprise one, two, or three nuclear localization signals (NLSs) independently selected from SEQ ID NOs: 366-369 and 371-384.

Embodiment 111 is the polynucleotide, composition or lipid nanoparticles of any one of Embodiments 89-110, wherein the fusion protein comprises, from N-terminus to C-terminus:
a. the first cleavase;
b. a peptide linker, optionally wherein the linker comprises 81 amino acid residues;
c. the second cleavase; and
d. an NLS comprising an SV40 NLS.

Embodiment 112 is the polynucleotide, composition or lipid nanoparticles of any one of Embodiments 89-111, wherein the fusion protein comprises, from N-terminus to C-terminus:
a. a first NLS, wherein the first NLS comprises an SV40 NLS;
b. the second cleavase;
c. a peptide linker, optionally wherein the peptide linker comprises 41 amino acids;
d. the first cleavase;
e. a second NLS, wherein the second NLS comprising an SV40 NLS.

Embodiment 113 is the polynucleotide, composition or lipid nanoparticles of any one of Embodiments 89-112, wherein the fusion protein comprises, from N-terminus to C-terminus:
a. the second cleavase;
b. a peptide linker, optionally wherein the peptide linker comprises 41 amino acids;
c. the first cleavase; and
d. an NLS, optionally wherein the NLS comprises an SV40 NLS.

Embodiment 114 is the polynucleotide, composition or lipid nanoparticles of any one of any one of Embodiments 89-113, wherein
(a) the fusion protein comprise an amino acid sequence of SEQ ID NOs: 3, 5, 7, 10, 13, 16, 40, 43, 45, 47, 50, 52, 55, 57, 60, 62, 65, 67, 70, 72, 75, 77, 80, 82, 85, 87, 90, 92, 101, or 105, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, 13, 16, 40, 43, 45, 47, 50, 52, 55, 57, 60, 62, 65, 67, 70, 72, 75, 77, 80, 82, 85, 87, 90, 92, 101, or 105; or
(b) the nucleic acid encoding the fusion protein comprises a nucleotide sequence of SEQ ID NOs: 1-2, 4, 6-8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104.

Embodiment 115 is the polynucleotide, composition or lipid nanoparticles of any one of Embodiments 89-114, wherein
(a) the fusion protein comprise an amino acid sequence of SEQ ID NOs: 3, 5, 7, 10, or 13, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, or 13; or
(b) the nucleic acid encoding the fusion protein comprises a nucleotide sequence of SEQ ID NOs: 1, 2, 4, 6, 8, 9, 11 or 12, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11 or 12.

Embodiment 116 is the polynucleotide, composition, or lipid nanoparticles of any one of Embodiments 89-115, wherein
(a) the fusion protein comprise an amino acid sequence of SEQ ID NOs: 5, 7, 10, or 13 or an amino acid sequence that is at least 85%, at least 90%, or at least 95%% identical to an amino acid sequence of SEQ ID NOs: 5, 7, 10, or 13; or
(b) the nucleic acid encoding the fusion protein comprises a nucleotide sequence of SEQ ID NOs: 4, 6, 8, 9, 11 or 12, a nucleotide sequence that is at least 85%, at least 90%, or at least 95% identical to SEQ ID NO: 4, 6, 8, 9, 11 or 12.

Embodiment 117 is the polynucleotide, composition, or lipid nanoparticles of Embodiment 116, wherein the first polypeptide comprises, from N-terminus to C-terminus:
a. the first intein;
b. the first cleavase, wherein the first cleavase is a S. pyogenes (Spy)Cas9 cleavase, said SpyCas9 cleavase comprising a R1333K mutation within its protospacer adjacent motif recognition domain; and
c. a first NLS comprising an SV40 NLS.

Embodiment 118 is the polynucleotide, composition, or lipid nanoparticles of Embodiment 116 or 117, wherein the second polypeptide comprises, from N-terminus to C-terminus:
a. a second NLS comprising an SV40 NLS;
b. a third NLS comprising a nucleoplasmin NLS;
c. the second cleavase;
d. a peptide linker, optionally wherein the peptide linker comprises 41 or 81 amino acid residues; and
e. the second intein capable of binding the first intein.

Embodiment 119 is the polynucleotide, composition, or lipid nanoparticles of any one of Embodiments 116-118, wherein the first polypeptide comprise an amino acid sequence of SEQ ID NOs: 28 or 31 or an amino acid sequence that is at least 85%, at least 90%, or at least 95% identical to SEQ ID NO: 28 or 31; or the nucleic acid or nucleic acids encoding the polypeptide or polypeptides comprises a sequence of SEQ ID NOs: 27 or 30, or a nucleotide sequence that is at least 85%, at least 90%, or at least 95% identical to SEQ ID NOs: 27 or 30.

Embodiment 120 is the polynucleotide, composition, or lipid nanoparticles of any one of Embodiments 116-119, wherein the second polypeptide comprise an amino acid sequence of SEQ ID NOs: 25 or 34 or an amino acid sequence that is at least 85%, at least 90%, or at least 95% identical to SEQ ID NO: 25 or 34; or the nucleic acid or nucleic acids encoding the polypeptide or polypeptides comprises a sequence of SEQ ID NOs: 24 or 33, or a nucleotide sequence that is at least 85%, at least 90%, or at least 95% identical to SEQ ID NOs: 24 or 33.

Embodiment 121 is the polynucleotide, composition or lipid nanoparticles of any one of Embodiments 89-120, wherein the polynucleotide comprises a 5′ UTR with at least 85%, at least 90%, or at least 95% identity to any one of SEQ ID NOs: 398-405.

Embodiment 122 is the polynucleotide, composition or lipid nanoparticles of any one of Embodiments 89-121, wherein the polynucleotide comprises a 3′ UTR with at least 85%, at least 90%, or at least 95% identity to any one of SEQ ID NOs: 406-413.

Embodiment 123 is the polynucleotide, composition or lipid nanoparticles of any one of Embodiments 89-122, wherein the polynucleotide comprises a 5′ UTR and 3′ UTR from the same source.

Embodiment 124 is the polynucleotide, composition or lipid nanoparticles of any one of Embodiments 89-123, wherein the polynucleotide comprises a 5′ cap, optionally wherein the 5′ cap is Cap0, Cap1, or Cap2.

Embodiment 125 is the polynucleotide, composition or lipid nanoparticles of any one of Embodiments 89-124, wherein the polynucleotide is an mRNA.

Embodiment 126 is the polynucleotide, composition or lipid nanoparticles of any one of Embodiments 89-125, wherein at least 85% of the uridine is substituted with a modified uridine.

Embodiment 127 is the polynucleotide, composition or lipid nanoparticles of Embodiment 126, wherein the modified uridine is one or more of N1-methyl-pseudouridine, pseudouridine or 5-iodouridine.

Embodiment 128 is the polynucleotide, composition or lipid nanoparticles of any one of Embodiments 126-127, wherein the modified uridine is N1-methyl-pseudouridine.

Embodiment 129 is the polynucleotide, composition or lipid nanoparticles of any one of Embodiments 126-128, wherein the modified uridine is pseudouridine.

Embodiment 130 is the polynucleotide, composition or lipid nanoparticles of Embodiment 126, wherein the modified uridine is 5-iodouridine.

Embodiment 131 is the polynucleotide, composition or lipid nanoparticles of any one of Embodiments 126-130, wherein at least 85% of the uridine is substituted with the modified uridine.

Embodiment 132 is the polynucleotide, composition or lipid nanoparticles of any one of Embodiments 126-131, wherein 100% uridine is substituted with the modified uridine.

Embodiment 133 is the composition or lipid nanoparticles of any one of Embodiments 90-132, wherein one or more of the nucleic acids encoding the fusion protein, the first guide RNA, and the second guide RNA are associated with one or more lipid nanoparticle (LNP).

Embodiment 134 is the composition or lipid nanoparticles of any one of Embodiments 90-133, wherein the nucleic acids encoding the fusion protein are each associated with a separate lipid nanoparticle (LNP).

Embodiment 135 is the composition or lipid nanoparticles of any one of Embodiments 90-133, wherein the first guide RNA and the second guide RNA are associated with a same lipid nanoparticle (LNP).

Embodiment 136 is the composition or lipid nanoparticles of any one of Embodiments 90-133, wherein all of the nucleic acids encoding the fusion protein, the first guide RNA, and the second guide RNA are associated with a same lipid nanoparticle (LNP).

Embodiment 137 is the composition or lipid nanoparticles of any one of Embodiments 133-136, wherein the LNP comprises (i) an ionizable lipid; (ii) a helper lipid; (iii) a stealth lipid; (iv) a neutral lipid; or combinations of one or more of (i)-(iv).

Embodiment 138 is the composition or lipid nanoparticles of Embodiment 137, wherein the ionizable lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 139 is the composition or lipid nanoparticles of any one of Embodiments 133-138, wherein the helper lipid is cholesterol.

Embodiment 140 is the composition or lipid nanoparticles of any one of Embodiments 133-139 wherein the stealth lipid is PEG-DMG.

Embodiment 141 is the composition or lipid nanoparticles of any one of Embodiments 133-140, wherein the PEG-DMG is 1,2-dimyristoyl-rac-glycero-3-methoxypolyethylene glycol-2000 (PEG2k-DMG).

Embodiment 142 is the composition or lipid nanoparticles of any one of Embodiments 133-141, wherein the neutral lipid is DSPC.

Embodiment 143 is the composition or lipid nanoparticles of any one of Embodiments 133-142, wherein the LNP composition comprises about 50 mol-% ionizable lipid; about 9 mol-% neutral lipid; about 3 mol-% of stealth lipid, and the remainder of the lipid component is helper lipid such as cholesterol.

Embodiment 144 is the composition or lipid nanoparticles of any one of Embodiments 133-143, wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

Embodiment 145 is a polypeptide encoded by the polynucleotide of any one of Embodiments 89-132.

Embodiment 146 is a vector comprising the polynucleotide of any one of Embodiments 89-132.

Embodiment 147 is an expression construct comprising a promoter operably linked to a sequence encoding the polynucleotide of any one of Embodiments 89-132.

Embodiment 148 is a plasmid comprising the expression construct of Embodiment 147.

Embodiment 149 is a host cell comprising the vector of Embodiment 146, the expression construct of Embodiment 147, or the plasmid of Embodiment 148.

Embodiment 150 is a pharmaceutical composition comprising the polynucleotide, composition, lipid nanoparticle, or polypeptide of any one of Embodiments 89-145, and a pharmaceutically acceptable carrier.

Embodiment 151 is a kit comprising the polynucleotide, composition, or polypeptide of any one of Embodiments 89-145.

Embodiment 152 is the use of the polynucleotide, composition, lipid nanoparticle or polypeptide any one of Embodiments 89-145 for producing a modification in the genome of a target cell.

Embodiment 153 is the use of the polynucleotide, composition, lipid nanoparticle or polypeptide any one of Embodiments 89-145 for the manufacture of a medicament for producing a modification in the genome of a target cell.

Embodiment 154 is the method of any one of Embodiments 1-85, wherein one or more of the nucleic acids encoding the fusion protein, the first guide RNA, and the second guide RNA are associated with one or more targeted LNP.

Embodiment 155 is the method of Embodiment 154, wherein the targeted LNP is targeted to one or more of the brain, eye, muscle, liver, lung, spleen, and bone marrow.

Embodiment 156 is the method of any one of Embodiments 154-155, wherein the targeted LNP comprises a targeting lipid component.

Embodiment 157 is the method of any one of Embodiments 154-156, wherein the targeted LNP comprises a targeting domain.

Embodiment 158 is the method of Embodiment 157, wherein the targeting domain comprises a nucleic acid, peptide, antibody, small molecule, glycan, sugar, or hormone.

Embodiment 159 is the method of any one of Embodiments 154-158, wherein the targeted LNP is administered by a delivery route of intravenous, intradermal, subcutaneous, inhalation, intranasal, or intramuscular delivery.

Embodiment 160 is the composition of any one of Embodiments 91 or 93-144, wherein one or more of the nucleic acids encoding the fusion protein, the first guide RNA, and the second guide RNA are associated with one or more targeted LNP.

Embodiment 161 is the composition of any one of Embodiments 160, wherein the targeted LNP comprises a targeting lipid component.

Embodiment 162 is the composition of any one of Embodiments 160-161, wherein the targeted LNP comprises a targeting domain.

Embodiment 163 is the composition of Embodiment 162, wherein the targeting domain comprises a nucleic acid, peptide, antibody, small molecule, glycan, sugar, or hormone.

### BRIEF DESCRIPTION OF THE DRAWINGS

FIG. 1 shows mean percent editing in HEK-Blue cells.

FIG. 2 shows percent editing in HEK-Blue cells.

FIG. 3 shows percent editing in primary mouse hepatocytes.

FIG. 4 shows percent editing in primary mouse hepatocytes.

FIG. 5 shows percent editing in Hepa 1-6 cells.

FIG. 6 shows percent editing in Hepa 1-6 cells.

FIG. 7 shows orthogonal Cas9-Cas9 fusion and SpyCas9 expression in Hepa 1-6 cells 72 hours post-transfection.

FIG. 8 shows orthogonal Cas9-Cas9 fusion and SpyCas9 expression relative to GAPDH.

FIG. 9 shows percent editing at the TTR locus in primary mouse hepatocytes.

FIG. 10 shows percent editing in liver tissue.

FIG. 11A shows serum TTR levels.

FIG. 11B shows serum TTR levels.

FIG. 12 shows percent editing in liver tissue.

FIG. 13A shows serum TTR levels.

FIG. 13B shows serum TTR levels.

FIG. 14 shows percent editing in transfected cells. The dotted line represents mRNA A excision.

FIG. 15 shows percent editing in transfected cells.

FIG. 16 shows orthogonal Cas9-Cas9 fusion protein expression as detected via Western blot.

FIG. 17 shows mean percent editing in primary mouse hepatocytes.

FIG. 18 shows mean percent editing at the PSCK9 locus by Spy guides and SpyCas9.

FIG. 19 shows mean percent editing at the PSCK9 locus by Nme guides and NmeCas9.

FIG. 20 shows mean percent editing at the PSCK9 locus by Spy guides and orthogonal Cas9-Cas9 fusion.

FIG. 21 shows mean percent editing at the PSCK9 locus by Nme guides and orthogonal Cas9-Cas9 fusion.

FIG. 22 shows mean percent editing at the PSCK9 locus by Spy guides and Nme guide G017566 and orthogonal Cas9-Cas9 fusion.

FIG. 23 shows mean percent editing at the PSCK9 locus by Spy guides and Nme G017564 and orthogonal Cas9-Cas9 fusion.

FIG. 24 shows mean percent editing at the TTR locus in primary mouse hepatocytes.

FIG. 25 shows mean percent editing at the TTR locus in primary mouse hepatocytes

FIG. 26 shows mean percent editing at the TTR locus in cells transfected with orthogonal Cas9-Cas9 fusion mRNA and Nme guides.

FIG. 27 shows mean percent editing at the TTR locus in cells transfected with NmeCas9 mRNA and Nme guides.

FIG. 28 shows mean percent editing at the TTR locus in cells transfected with Spy guide G000502, Nme guide G021845, and orthogonal Cas9-Cas9 fusion.

FIG. 29 shows mean percent editing at the TTR locus in cells transfected with Spy guide G000502, Nme guide G021846, and orthogonal Cas9-Cas9 fusion.

FIG. 30 shows mean percent editing at the TTR locus in cells transfected with Spy guide G000502 and SpyCas9.

FIG. 31 shows mean percent editing at the TTR locus in cells transfected with Nme guide G021845 and NmeCas9.

FIG. 32 shows mean percent editing at the TTR locus in cells transfected with Nme guide G021846 and NmeCas9.

### BRIEF DESCRIPTION OF DISCLOSED SEQUENCES

| SEQ ID | |
|---|---|
| NO | Description |
| 1 | Cas9-Cas9 fusion (mRNA A)-mRNA sequence |
| 2 | Cas9-Cas9 fusion (mRNA A)-ORF sequence |
| 3 | Cas9-Cas9 fusion (mRNA A)-amino acid sequence |
| 4 | Cas9-Cas9 fusion (mRNA A with HiBit)-ORF sequence |
| 5 | Cas9-Cas9 fusion (mRNA A with HiBit)-amino acid sequence |
| 6 | Cas9-Cas9 fusion (mRNA B)-ORF sequence |
| 7 | Cas9-Cas9 fusion (mRNA B)- amino acid sequence |
| 8 | Cas9-Cas9 fusion (mRNA B with HiBit)-mRNA sequence |
| 9 | Cas9-Cas9 fusion (mRNA B with HiBit)-ORF sequence |
| 10 | Cas9-Cas9 fusion (mRNA B with HiBit)-amino acid sequence |
| 11 | Cas9-Cas9 fusion (mRNA C)-mRNA sequence |
| 12 | Cas9-Cas9 fusion (mRNA C)-ORF sequence |
| 13 | Cas9-Cas9 fusion (mRNA C)-amino acid sequence |
| 14 | Cas9-Cas9 fusion (mRNA D)-mRNA sequence |
| 15 | Cas9-Cas9 fusion (mRNA D)-ORF sequence |
| 16 | Cas9-Cas9 fusion (mRNA D)-amino acid sequence |
| 17 | SpyCas9 (mRNA E) -mRNA sequence |
| 18 | SpyCas9 (mRNA E) -ORF sequence |
| 19 | SpyCas9 (mRNA E) -amino acid sequence |
| 20 | Nme2Cas9 (mRNA F) -mRNA sequence |
| 21 | Nme2Cas9 (mRNA F) -ORF sequence |
| 22 | Nme2Cas9 (mRNA F) -amino acid sequence |
| 23 | Nme2 Cas9 Intein (mRNA G) - mRNA sequence |
| 24 | Nme2 Cas9 Intein (mRNA G) - ORF sequence |
| 25 | Nme2 Cas9 Intein (mRNA G) - amino acid sequence |
| 26 | Spy-R1333K Cas9 Intein (mRNA H) -mRNA sequence |
| 27 | Spy-R1333K Cas9 Intein (mRNA H) -ORF sequence |
| 28 | Spy-R1333K Cas9 Intein (mRNA H) -amino acid sequence |
| 29 | Spy-R1333K Cas9 Intein (mRNA I) -mRNA sequence |
| 30 | Spy-R1333K Cas9 Intein (mRNA I) -ORF sequence |
| 31 | Spy-R1333K Cas9 Intein (mRNA I) -amino acid sequence |
| 32 | Nme2 Cas9 Intein (mRNA J) - mRNA sequence |
| 33 | Nme2 Cas9 Intein (mRNA J) - ORF sequence |
| 34 | Nme2 Cas9 Intein (mRNA J) - amino acid sequence |
| 35 | SpyCas9 with HiBit (mRNA K) -mRNA sequence |
| 36 | SpyCas9 with HiBit (mRNA K) -ORF sequence |
| 37 | SpyCas9 with HiBit (mRNA K) -amino acid sequence |
| 38 | Cas9-Cas9 fusion (mRNA L)-mRNA sequence |
| 39 | Cas9-Cas9 fusion (mRNA L)-ORF sequence |
| 40 | Cas9-Cas9 fusion (mRNA L)-amino acid sequence |
| 41 | Cas9-Cas9 fusion with HiBit (mRNA M)-mRNA sequence |
| 42 | Cas9-Cas9 fusion with HiBit (mRNA M)-ORF sequence |
| 43 | Cas9-Cas9 fusion with HiBit (mRNA M)-amino acid sequence |
| 44 | Cas9-Cas9 fusion (mRNA M)-ORF sequence |
| 45 | Cas9-Cas9 fusion (mRNA M)-amino acid sequence |
| 46 | R1333 WT Cas9-Cas9 fusion PA1-4 (mRNA N) - ORF sequence (Example 4) |
| 47 | R1333 WT Cas9-Cas9 fusion PA1-4 (mRNA N) - amino acid sequence |
| | (Example 4) |
| 48 | Cas9-Cas9 fusion-71 amino acid residue linker and HiBit tag (mRNA O)-mRNA |
| | sequence |
| 49 | Cas9-Cas9 fusion-71 amino acid residue linker and HiBit tag (mRNA O)-ORF |
| | sequence |
| 50 | Cas9-Cas9 fusion-71 amino acid residue linker and HiBit tag (mRNA O)-amino |
| | acid sequence |
| 51 | Cas9-Cas9 fusion-71 amino acid residual linker (mRNA O)-mRNA sequence |
| 52 | Cas9-Cas9 fusion-71 amino acid residual linker (mRNA O)-amino acid sequence |
| 53 | Cas9-Cas9 fusion-61 amino acid residue linker and HiBit tag (mRNA P)-mRNA |
| | sequence |
| 54 | Cas9-Cas9 fusion-61 amino acid residue linker and HiBit tag (mRNA P)-ORF |
| | sequence |
| 55 | Cas9-Cas9 fusion-61 amino acid residue linker and HiBit tag (mRNA P)-amino |
| | acid sequence |
| 56 | Cas9-Cas9 fusion-61 amino acid residual linker (mRNA P)-mRNA sequence |
| 57 | Cas9-Cas9 fusion-61 amino acid residual linker (mRNA P)-amino acid sequence |
| 58 | Cas9-Cas9 fusion-51 amino acid residue linker and HiBit tag (mRNA Q)-mRNA |
| | sequence |
| 59 | Cas9-Cas9 fusion-51 amino acid residue linker and HiBit tag (mRNA Q)-ORF |
| | sequence |
| 60 | Cas9-Cas9 fusion-51 amino acid residue linker and HiBit tag (mRNA Q)-amino |
| | acid sequence |
| 61 | Cas9-Cas9 fusion-51 amino acid residual linker (mRNA Q)-ORF sequence |
| 62 | Cas9-Cas9 fusion-51 amino acid residual linker (mRNA Q)-amino acid sequence |
| 63 | Cas9-Cas9 fusion-41 amino acid residue linker and HiBit tag (mRNA R)-mRNA |
| | sequence |
| 64 | Cas9-Cas9 fusion-41 amino acid residue linker and HiBit tag (mRNA R)-ORF |
| | sequence |
| 65 | Cas9-Cas9 fusion-41 amino acid residue linker and HiBit tag (mRNA R)-amino |
| | acid sequence |
| 66 | Cas9-Cas9 fusion-41 amino acid residual linker (mRNA R)-ORF sequence |
| 67 | Cas9-Cas9 fusion-41 amino acid residual linker (mRNA R)-amino acid sequence |
| 68 | Cas9-Cas9 fusion-31 amino acid residue linker and HiBit tag (mRNA S)-mRNA |
| | sequence |
| 69 | Cas9-Cas9 fusion-31 amino acid residue linker and HiBit tag (mRNA S)-ORF |
| | sequence |
| 70 | Cas9-Cas9 fusion-31 amino acid residue linker and HiBit tag (mRNA S)-amino |
| | acid sequence |
| 71 | Cas9-Cas9 fusion-31 amino acid residual linker (mRNA S)-ORF sequence |
| 72 | Cas9-Cas9 fusion-31 amino acid residual linker (mRNA S)-amino acid sequence |
| 73 | Cas9-Cas9 fusion-21 amino acid residue linker and HiBit tag (mRNA T)-mRNA |
| | sequence |
| 74 | Cas9-Cas9 fusion-21 amino acid residue linker and HiBit tag (mRNA T)-ORF |
| | sequence |
| 75 | Cas9-Cas9 fusion-21 amino acid residue linker and HiBit tag (mRNA T)- amino |
| | acid sequence |
| 76 | Cas9-Cas9 fusion-21 amino acid residual linker (mRNA T)-ORF sequence |
| 77 | Cas9-Cas9 fusion-21 amino acid residual linker (mRNA T)-amino acid sequence |
| 78 | Cas9-Cas9 fusion-11 amino acid residue linker and HiBit tag (mRNA U)-mRNA |
| | sequence |
| 79 | Cas9-Cas9 fusion-11 amino acid residue linker and HiBit tag (mRNA U)-ORF |
| | sequence |
| 80 | Cas9-Cas9 fusion-11 amino acid residue linker and HiBit tag (mRNA U)- |
| | Amino acid sequence |
| 81 | Cas9-Cas9 fusion-11 amino acid residual linker (mRNA U)-ORF sequence |
| 82 | Cas9-Cas9 fusion-11 amino acid residual linker (mRNA U)-amino acid sequence |
| 83 | Cas9-Cas9 fusion-81 amino acid residue linker and HiBit tag-SpyCas9 without |
| | Rec2 domain (mRNA V) -mRNA sequence |
| 84 | Cas9-Cas9 fusion-81 amino acid residue linker and HiBit tag-SpyCas9 without |
| | Rec2 domain (mRNA V) -ORF sequence |
| 85 | Cas9-Cas9 fusion-81 amino acid residue linker and HiBit tag-SpyCas9 without |
| | Rec2 domain (mRNA V) -amino acid sequence |
| 86 | Cas9-Cas9 fusion-81 amino acid residual linker withSpyCas9 without Rec2 |
| | domain (mRNA V) -mRNA sequence |
| 87 | Cas9-Cas9 fusion-81 amino acid residual linker with SpyCas9 without Rec2 |
| | domain (mRNA V) -amino acid sequence |
| 88 | Cas9-Cas9 fusion-81 amino acid residue linker and HiBit tag-SpyCas9 without |
| | Rec3 domain (mRNA W) -mRNA sequence |
| 89 | Cas9-Cas9 fusion-81 amino acid residue linker and HiBit tag-SpyCas9 without |
| | Rec3 domain (mRNA W) -ORF sequence |
| 90 | Cas9-Cas9 fusion-81 amino acid residue linker and HiBit tag-SpyCas9 without |
| | Rec3 domain (mRNA W) -amino acid sequence |
| 91 | Cas9-Cas9 fusion-81 amino acid residual linker with SpyCas9 without Rec3 |
| | domain (mRNA W) -mRNA sequence |
| 92 | Cas9-Cas9 fusion-81 amino acid residual linker with SpyCas9 without Rec3 |
| | domain (mRNA W) -amino acid sequence |
| 93 | SpyCas9 (mRNA X) -mRNA sequence |
| 94 | SpyCas9 (mRNA X) -ORF sequence |
| 95 | SpyCas9 (mRNA X) -amino acid sequence |
| 96 | SpyCas9-Nme cleavase fusion ORF |
| 97 | SpyCas9-Nme cleavase fusion amino acid sequence |
| 98 | R1335S Spy Cas9-WT NmeCas9 fusion ORF |
| 99 | R1335S Spy Cas9-WT NmeCas9 fusion amino acid sequence |
| 100 | R1333K Spy-WT NmeCas9 fusion ORF |
| 101 | R1333K Spy -WT NmeCas9 fusion amino acid sequence |
| 102 | WT SpyCas9 -WT NmeCas9 fusion ORF |
| 103 | WT SpyCas9-WT NmeCas9 fusion ORF |
| 104 | Spy Cas9 R133K nucleic acid coding sequence without start or stop codons |
| | suitable for fusion constructs |
| 105 | Amino acid sequence for Spy Cas9 R1333K suitable for fusion constructs |
| 106 | WT Nme1 Cas9 coding sequence |
| 107 | WT Nme1 Cas9 amino acid sequence |
| 108 | Nme2 Cas9 coding sequence |
| 109 | Nme2 Cas9 amino acid sequence |
| 110 | WT SpyCas9 ORF |
| 111 | WT SpyCas9 amino acid sequence |
| 112-119 | NOT USED |
| 120 | Exemplary amino acid sequence of Nme1Cas9 cleavase |
| 121 | Exemplary coding sequence encoding Nme1Cas9 cleavase |
| 122 | Exemplary coding sequence encoding Nme1Cas9 cleavase |
| 123 | Exemplary coding sequence encoding Nme1Cas9 cleavase |
| 124 | Exemplary open reading frame for Nme1Cas9 cleavase |
| 125 | Exemplary open reading frame for Nme1Cas9 cleavase |
| 126 | Exemplary open reading frame for Nme1Cas9 cleavase |
| 127 | Exemplary amino acid sequence of Nme3Cas9 cleavase |
| 128 | Exemplary coding sequence encoding Nme3Cas9 cleavase |
| 129 | Exemplary coding sequence encoding Nme3Cas9 cleavase |
| 130 | Exemplary coding sequence encoding Nme3Cas9 cleavase |
| 131 | Exemplary open reading frame for Nme3Cas9 cleavase |
| 132 | Exemplary open reading frame for Nme3Cas9 cleavase |
| 133 | Exemplary open reading frame for Nme3Cas9 cleavase |
| 134 | Exemplary coding sequence encoding Nme2Cas9 (mRNA AA) |
| 135 | Exemplary coding sequence encoding Nme1Cas9 (mRNA AB) |
| 136 | Exemplary amino acid sequence for Nme2Cas9 (mRNA AA amino acid) |
| 137 | Exemplary amino acid sequence for Nme1Cas9 (mRNA AB amino acid) |
| 138 | Exemplary open reading frame for Nme2Cas9 |
| 139 | Exemplary open reading frame for Nme1Cas9 |
| 140 | WT Smu Cas9 coding sequence |
| 141 | human codon-optimized Smu Cas9 coding sequences |
| 142 | WT Smu Cas9 amino acid sequence |
| 143 | WT Cje Cas9 coding sequence |
| 144 | WT Cje Cas9 amino acid sequence |
| 145-149 | NOT USED |
| 150 | Exemplary amino acid residue linker (81 aa) |
| 151 | Exemplary amino acid residue linker (71 aa) |
| 152 | Exemplary amino acid residue linker (61 aa) |
| 153 | Exemplary amino acid residue linker (51 aa) |
| 154 | Exemplary amino acid residue linker (41 aa) |
| 155 | Exemplary amino acid residue linker (31 aa) |
| 156 | Exemplary amino acid residue linker (21 aa) |
| 157 | Exemplary amino acid residue linker (11 aa) |
| 158 | Exemplary amino acid residue linker (81 aa) |
| 159 | Exemplary SpyCas9 sgRNA-1 (Table 39) |
| 160-167 | Exemplary Spy Guide RNA Conserved Region only- unmodified sequences |
| 168-169 | NOT USED |
| 170-177 | Exemplary Spy Guide RNA unmodified sequence |
| 178-179 | NOT USED |
| 180-191 | Exemplary Spy Guide RNA conserved region only-modified sequences |
| 192-194 | Exemplary Spy Guide RNA -modified sequence |
| 195-199 | NOT USED |
| 200-237 | Exemplary Spy guide RNA sequences |
| 238 | Exemplary Nme guide; plCR012468 |
| 239 | Exemplary Nme guide; plCR012469 |
| 240-253 | Exemplary Nme sgRNA sequences |
| 254 | Exemplary Spy guide; plG000665 |
| 255 | Exemplary Spy guide; plG012746 |
| 256 | Exemplary Spy guide; plG012684 |
| 257 | Exemplary Spy guide; plG012685 |
| 258 | Exemplary Spy guide; plG012686 |
| 259 | Exemplary Spy guide; plG012687 |
| 260 | Exemplary Spy guide; plG012688 |
| 261-269 | Target sequences for Spy guides encoded by the exemplary plasmids |
| 270-271 | NOT USED |
| 272-278 | Exemplary Nme Guide RNA comprising linkers |
| 279 | Exemplary Nme sgRNA-1 (Table 40) |
| 280-283 | Exemplary Nme Guide RNA Conserved Region only- unmodified sequences |
| 284-292 | Exemplary Nme Guide RNA Conserved Region only- modified sequences |
| 293-297 | Exemplary Nme Guide RNA modified sequences |
| 298-300 | NOT USED |
| 301-365 | Exemplary linkers |
| 366-397 | Exemplary NLS amino acid sequences and coding sequences |
| 398-405 | Exemplary 5′ UTR sequences |
| 406-413 | Exemplary 3′ UTR sequences |
| 414 | Exemplary Kozak sequence |
| 415 | Exemplary Kozak sequence |
| 416 | Exemplary poly-A sequence |

### DETAILED DESCRIPTION

The present disclosure provides, e.g., systems and methods of contacting a cell with an orthogonal Cas9-Cas9 fusion for precise and reproducible genome editing. The methods provide, for example, excising sequences from the genome of a cell without significant side effects such as inversions.

In some embodiments, provided herein is a method of genetically modifying a cell, comprising contacting the cell with an orthogonal Cas9-Cas9 fusion comprising a first cleavase and a second cleavase, or a nucleic acid encoding the same, thereby excising a DNA sequence between a first cleavage site cleaved by the first Cas9 cleavase and a second cleavage site cleaved by the second Cas9 cleavase.

The present disclosure also relates to manufacturing methods to prepare cells in vitro for subsequent therapeutic administration to a subject. In some embodiments, the platform relates to genome editing via simultaneous or sequential administration of lipid nanoparticles (LNPs) comprising an orthogonal Cas9-Cas9 fusion comprising a first cleavase and a second cleavase, or a nucleic acid encoding the same as disclosed herein. The systems and methods disclosed herein are relevant to any cell type but is particularly advantageous in preparing cells that require excision of a defined genomic sequence for full therapeutic applicability, e.g., in primary immune cells. As provided herein, the platform methods apply to “a cell” or to “a cell population” (or “population of cells”). When referring to delivery or gene editing methods for “a cell” herein, it is understood that the methods may be used for delivery or gene editing to “a cell population.”

In some embodiments, provided herein is a cell treated in vitro with any method or composition disclosed herein. In some embodiments, provided herein is a cell treated in vivo with any method or composition disclosed herein. In some embodiments, provided herein is a population of cells comprising any cell disclosed herein.

In some embodiments, provided herein is use of any cell, population of cells, or composition disclosed herein for treating cancer. In some embodiments, provided herein is use of any cell, population of cells, or composition disclosed herein for preparation of a medicament for treating cancer. In some embodiments, provided herein is an engineered cell modified by the methods disclosed herein, and the engineered cell comprises at least one genomic modification, e.g., deletion of contiguous nucleotides.

In some embodiments, the orthogonal Cas9-Cas9 fusion disclosed herein comprises a first Cas9 cleavase and a second Cas9 cleavase. The first cleavase may be a S. pyogenes (Spy)Cas9 cleavase, and the SpyCas9 cleavase comprising an R1333K mutation within its protospacer adjacent motif recognition domain. The second cleavase may be an N. meningitidis (Nme)Cas9 cleavase, a C. jejuni (Cje) Cas9 cleavase, or a Simonsiella muelleri (Smu) Cas9 cleavase.

Accordingly, in some embodiments, a method of producing a modification in the genome of a target cell is provided. In some embodiments, the method comprises contacting the cell with: (a) a fusion protein, or a nucleic acid encoding a fusion protein, wherein the fusion protein comprises a first cleavase and a second cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, a method of producing a cell or a population of cells is provided. In some embodiments, the method comprises a modification in the genome of the target cell or cells, the method comprising contacting the cell or cells with: (a) a fusion protein, or a nucleic acid encoding a fusion protein, wherein the fusion protein comprises a first cleavase and a second cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, a composition is provided, the composition comprising (a) a polynucleotide comprising an open reading frame (ORF) encoding a fusion protein, wherein the fusion protein comprises a first cleavase and a second cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, a composition is provided, the composition comprising (a) a first polynucleotide comprising an ORF encoding the first polypeptide, wherein the first polypeptide comprises first cleavase and a first intein, wherein the first cleavase is a S. pyogenes (Spy)Cas9 cleavase, said SpyCas9 cleavase comprising a R1333K mutation within its protospacer adjacent motif recognition domain; (b) a second polynucleotide comprising an ORF encoding the second polypeptide, wherein the second polypeptide comprises a second cleavase and a second intein capable of binding the first intein, wherein the second cleavase is a N. meningitidis (Nme)Cas9 cleavase, a C. jejuni (Cje) Cas9 cleavase, or a Simonsiella muelleri (Smu) Cas9 cleavase; and wherein the first polypeptide binds to the second polypeptide through intein catalysis.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy, 2′ halide, or 2′-O-(2-methoxyethyl) (2′-O-moe) substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Nucleic acid includes “unlocked nucleic acid” enables the modulation of the thermodynamic stability and also provides nuclease stability. RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with the target sequence and the agent binds to the target sequence; in cases where the agent is a cleavase or nickase, binding can be followed by cleaving or nicking.

As used herein, “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases, Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “orthogonal” refers to any two genomic editors (e.g., base editors, nucleases, nickases, or cleavases) where each is capable of recognizing its own target(s) via its cognate guide RNA(s) but not compatible with the guide RNA(s) cognate to the other genomic editor, e.g., each is not capable of recognizing the target(s) of the other genomic editor via the guide RNA(s) cognate to the other genomic editor. For example, an N. meningitidis Cas9 (NmeCas9) cleavase may be capable of recognizing a genomic locus via a guide RNA cognate to the NmeCas9 cleavase, and an S. pyogenes Cas9 (SpyCas9) cleavase may be capable of recognizing another genomic locus via a guide RNA cognate to the SpyCas9 cleavase. In this example, the NmeCas9 cleavase and the SpyCas9 cleavase are orthogonal to each other. Genome editors or genome editing components may be engineered to be orthogonal. Although in this example, the NmeCas9 cleavase and the SpyCas9 cleavase are derived from different organisms, two genomic editors need not be derived from different organisms to be orthogonal to each other.

As used herein, the term “fusion protein” refers to a hybrid polypeptide which comprises polypeptides from at least two different proteins or sources. One polypeptide may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “intein,” as used herein refers to a protein domain capable of mediating a process known as protein splicing. For example, a first intein positioned at the C-terminus of a first polypeptide (the N-terminal intein) and a second intein positioned at the N-terminus of a second polypeptide (the C-terminal intein) may undergo a chemical reaction culminating in the formation of a peptide bond between the first polypeptide and the second polypeptide and the excision of the first and second intein domains. Intein-mediated protein splicing is known in the art, and represents a well-established technique for generating a single protein from two separate polypeptides (See, e.g., Shah and Muir. Inteins: Nature's Gift to Protein Chemists. Chem. Sci., 5(1), 446-461 (2014)).

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is a peptide linker comprising an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 301), SGSETPGTSESA (SEQ ID NO: 302), or SGSETPGTSESATPEGGSGGS (SEQ ID NO: 303). In some embodiments, the linker comprises one or more sequences selected from SEQ ID NOs: 150-158 and 304-365.

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be fused to the molecule by a covalent bond, hydrogen bonds or ionic interactions. In some embodiments, the NLS may be fused to the molecule via a linker.

As used herein, “open reading frame” or “ORF” of a gene refers to a sequence consisting of a series of codons that specify the amino acid sequence of the protein that the gene codes for. The ORF generally begins with a start codon (e.g., ATG in DNA or AUG in RNA) and ends with a stop codon, e.g., TAA, TAG or TGA in DNA or UAA, UAG, or UGA in RNA.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise one or more modifications, e.g., as provided below. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in or accompanying this disclosure can be a uridine or a modified uridine.

As used herein, the “minimal uridine codon(s)” for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating uridine content.

As used herein, the “uridine dinucleotide (UU) content” of an ORF can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating uridine dinucleotide content.

As used herein, the “minimal adenine codon(s)” for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating adenine content.

As used herein, the “adenine dinucleotide content” of an ORF can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating adenine dinucleotide content.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, the term “genomic locus,” when used in the context of a genomic locus being targeted by a guide RNA, includes one or more parts of a genome, the targeting of which affects the expression of the gene that is associated with the locus. For example, a genomic locus may include a coding sequence of a gene, an intron sequence of a gene, a regulatory sequence, a transcriptional control sequence of a gene, a translational control sequence of a gene, a splicing site, or a non-coding sequence between genes (e.g., intergenic space).

As used herein, a “guide sequence” or “guide region” or “targeting sequence” or “spacer” or “spacer sequence” and the like refers to a sequence within a gRNA that is complementary to a target sequence and functions to direct a gRNA to a target sequence for binding or modification (e.g., cleavage) by an RNA-guided nickase. A guide sequence can be 20 nucleotides in length, e.g., in the case of Streptococcus pyogenes (i.e., Spy Cas9 (also referred to as SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. A guide sequence can be 20-25 nucleotides in length, e.g., in the case of Nme Cas9, e.g., 20-, 21-, 22-, 23-, 24- or 25-nucleotides in length. For example, a guide sequence of 24 nucleotides in length can be used with Nme Cas9, e.g., Nme2 Cas9.

In some embodiments, the target sequence is in a genomic locus or on a chromosome, for example, and is complementary to the guide sequence. In some embodiments, the degree of complementarity or identity between a guide sequence and its corresponding target sequence may be about 75%, about 80%, about 85%, about 90%, about 95%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, at least 18, at least 19, at least 20 or more base pairs. In some embodiments, the guide sequence and the target region may contain 1-4 mismatches where the guide sequence comprises at least 17, at least 18, at least 19, at least 20 or more nucleotides. In some embodiments, the guide sequence and the target region may contain 1, 2, 3, or 4 mismatches where the guide sequence comprises 20 nucleotides. In some embodiments, the degree of complementarity or identity between a guide sequence and its corresponding target sequence is at least 80%, at least 85%, at least 90%, or at least 95%, for example when, the guide sequence comprises a sequence 24 contiguous nucleotides. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides.

As used herein, a “target sequence” or “genomic target sequence” refers to a sequence of nucleic acid in a target genomic locus, in either the positive or the negative strand, that has complementarity to the guide sequence of the gRNA, i.e., that is sufficiently complementary to the guide sequence of the gRNA to permit specific binding of the guide to the target sequence. The interaction of the target sequence and the guide sequence directs an RNA-guided DNA binding agent to bind, and potentially nick or cleave (depending on the activity of the agent), within the target sequence. The specific length of the target sequence and the number of mismatches possible between the target sequence and the guide sequence depend, for example, on the identity of the Cas9 nuclease being directed by the gRNA. Target sequences for Cas proteins include both the positive and negative strands of genomic DNA (i.e., the sequence given and the sequence's reverse complement), as a nucleic acid substrate for a Cas protein is a double stranded nucleic acid. Accordingly, where a guide sequence is said to be “complementary to a target sequence,” it is to be understood that the guide sequence may direct an RNA-guided DNA binding agent (e.g., dCas9 or impaired Cas9) to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, a first sequence is considered to “comprise a sequence that is at least X % identical to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, the term “contact” refers to providing at least one component so that the component physically contacts a cell, including physically contacting the cell surface, cytosol, and/or nucleus of the cell. “Contacting” a cell with a polypeptide encompasses, for example, contacting the cell with a nucleic acid that encodes the polypeptide and allowing the cell to express the polypeptide.

As used herein, “indel” refers to an insertion or deletion mutation consisting of a number of nucleotides that are either inserted, deleted, or inserted and deleted, e.g., at the site of double-strand breaks (DSBs), in a target nucleic acid. As used herein, when indel formation results in an insertion, the insertion is a random insertion at the site of a DSB and is not generally directed by or based on a template sequence.

As used herein, an “excision” is defined as a single long deletion that starts within the indel window of one guide RNA and ends within the indel window of the other guide RNA.

As used herein, “inversion” refers to a mutation in which a DNA sequence flanked by two double-strand breaks (DSBs) is reinserted back into the chromosome in the opposite orientation. Inversion may occur following the simultaneous or near-simultaneous generation of two adjacent D SBs.

As used herein, “reduces or eliminates” (or “reduced or eliminated”) expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art. As used herein, “eliminated” expression is understood as a reduction of expression to below the level of detection of the protein by the method used.

As used herein, “increased” (or “increases”) expression of a protein on a cell refers to an increase in expression of the protein relative to an unmodified cell. In some embodiments, the surface expression of a protein on a cell is measured by flow cytometry and has “increased” surface expression relative to an unmodified cell as evidenced by an increase in fluorescence signal upon staining with the same antibody against the protein. The “increase” of protein expression can be measured by other known techniques in the field with appropriate controls known to those skilled in the art.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, the methods of the disclosure “knockout” a target protein one or more cells (e.g., in a population of cells including in vivo populations such as those found in tissues). In some embodiments, a knockout is not the formation of mutant of the target protein, for example, created by indels, but rather the complete loss of expression of the target protein in a cell, i.e., decrease of expression to below the level of detection of the assay used.

As used herein, a “population of cells comprising edited cells” (or “population of cells comprising engineered cells”) or the like refers to a cell population that comprises edited cells (or engineered cells), however not all cells in the population must be edited. A cell population comprising edited cells may also include non-edited cells. The percentage of edited cells within a cell population comprising edited cells may be determined by counting the number of cells within the population that are edited in the population as determined by standard cell counting methods. For example, in some embodiments, a cell population comprising edited cells comprising a single genome edit will have at least 20%, at least 30%, at least 40%, preferably at least 50%, at least 60%, at least 70%, at least 80%, at least 90%, at least 95%, or at least 99% of the cells in the population with the single edit. In some embodiments, a cell population comprising edited cells comprising at least two genome edits will have at least 20%, at least 30%, at least 40%, at least 50%, at least 60%, at least 70%, at least 80%, at least 90%, or at least 95% of the cells in the population with at least two genome edits.

As used herein, “TTR” refers to the TTR gene (NCBI Gene ID: 7276; Ensembl: ENSG00000118271), which encodes the protein transthyretin (TTR).

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including reoccurrence of the symptom.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

As used herein, a “subject” refers to any member of the animal kingdom. In some embodiments, “subject” refers to humans. In some embodiments, “subject” refers to non-human animals. In some embodiments, “subject” refers to primates. In some embodiments, a subject may be a transgenic animal, genetically engineered animal, or a clone. In certain embodiments of the present invention the subject is an adult, an adolescent, or an infant. In some embodiments, terms “individual” or “patient” are used and are intended to be interchangeable with “subject”.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1% or within two standard deviations of a set of values. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or,” unless the context clearly indicates otherwise.

As used herein, ranges include both the upper and lower limit.

In the event of a conflict between a sequence in the application and an indicated accession number or position in an accession number, the sequence in the application predominates.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

### II. Cas9-Cas9 Fusion Protein

The Cas9-Cas9 fusion system may use both Cas9 domains to achieve coordinated cleavage at two neighboring positions within the genome. For example, attenuated SpyCas9 cleavase can be coupled to NmeCas9 cleavase to allow the formation two double-strand breaks together and provide clear excision of the intervening sequence.

In some embodiments, the fusion protein (or “Cas9-Cas9 fusion”) disclosed herein comprises (a) a first Cas9 cleavase and a guide RNA (gRNA) that targets at least one genomic locus and that is cognate to the first Cas9 cleavase; and (b) a second Cas9 cleavase and a gRNA that targets at least one genomic locus and that is cognate to the second Cas9 cleavase, thereby excising a DNA sequence between a first cleavage site cleaved by the first cleavase and a second cleavage site cleaved by the second cleavase. In some embodiments, the fusion protein is delivered to the cell as at least one polypeptide or at least one mRNA.

In some embodiments, a polynucleotide comprising an open reading frame (ORF) encoding a fusion protein is provided. In some embodiments, the fusion protein comprises a first cleavase and a second cleavase, wherein: the first cleavase is a S. pyogenes (Spy)Cas9 cleavase, said SpyCas9 cleavase comprising a R1333K mutation within its protospacer adjacent motif recognition domain; and the second cleavase is a N. meningitidis (Nme)Cas9 cleavase, a C. jejuni (Cje) Cas9 cleavase, or a Simonsiella muelleri (Smu) Cas9 cleavase.

In some embodiments, a composition is provided, the composition comprising (a) a polynucleotide comprising an open reading frame (ORF) encoding a fusion protein, wherein the fusion protein comprises a first cleavase and a second cleavase, wherein: the first cleavase is a S. pyogenes (Spy)Cas9 cleavase, said SpyCas9 cleavase comprising a R1333K mutation within its protospacer adjacent motif recognition domain; and the second cleavase is a N. meningitidis (Nme)Cas9 cleavase, a C. jejuni (Cje) Cas9 cleavase, or a Simonsiella muelleri (Smu) Cas9 cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, a composition is provided, the composition comprising (a) a first polynucleotide comprising an ORF encoding the first polypeptide, wherein the first polypeptide comprises first cleavase and a first intein, wherein the first cleavase is a S. pyogenes (Spy)Cas9 cleavase, said SpyCas9 cleavase comprising a R1333K mutation within its protospacer adjacent motif recognition domain; (b) a second polynucleotide comprising an ORF encoding the second polypeptide, wherein the second polypeptide comprises a second cleavase and a second intein capable of binding the first intein, wherein the second cleavase is a N. meningitidis (Nme)Cas9 cleavase, a C. jejuni (Cje) Cas9 cleavase, or a Simonsiella muelleri (Smu) Cas9 cleavase; and wherein the first polypeptide binds to the second polypeptide through intein catalysis.

In some embodiments, the first cleavase is located N-terminal to the second cleavase. In some embodiments, the first cleavase is located C-terminal to the second cleavase.

### A. First Cleavase: Attenuated SpyCas9

In some embodiment, the present disclose provides an attenuated SpCas9 comprising a mutation in its protospacer adjacent motif (PAM) recognition domain. In some embodiments, the SpyCas9 may have an attenuated DNA-binding activity. Exemplary mutations in the PAM-interacting domain include R1333S, R1333K, and R1335K. See also WO2016106338; Nishimasu et al., Crystal structure of Cas9 in complex with guide RNA and target DNA. Cell. 2014 Feb. 27; 156(5):935-49; and Anders et al., Structural basis of PAM-dependent target DNA recognition by the Cas9 endonuclease. Nature. 2014 Sep. 25; 513(7519):569-73, the contents of all of which are incorporated by reference herein.

In some embodiments, the first cleavase comprises a R1333K mutation within its PAM recognition domain.

In some embodiments, the SpyCas9 cleavase comprises an amino acid sequence of SEQ ID NO: 105 or an amino acid sequence that is at least 85%, at least 90%, or at least 95% identical to SEQ ID NO: 105. In some embodiments, the nucleotide encoding the SpyCas9 cleavase comprises an open reading frame (ORF) comprising a sequence of SEQ ID NO: 104 or a nucleotide sequence that is at least 85, at least 90%, or at least 95% identical to SEQ ID NO: 104.

### B. Second Cas9 Cleavase

In some embodiments, the fusion protein disclosed herein comprises a second Cas9 cleavase. The second Cas9 may be Class II-C Cas9 orthologs. Non-limiting examples of Class II-C Cas9s include N. meningitidis (NmeCas9), C. jejuni Cas9 (CjeCas9), or S. muelleri (Smu) Cas9.

In some embodiments, the Cas9 is an Nme1Cas9, an Nme2Cas9, an Nme3Cas9. In some embodiments, the second cleavase is a NmeCas9 cleavase. In some embodiments, the second cleavase is an Nme1Cas9, an Nme2Cas9, or an Nme3Cas9. In some embodiments, the second cleavase is an Nme2Cas9.

In some embodiments, the second cleavase is a CjeCas9. In some embodiments, the second cleavase is a SmuCas9.

In some embodiments, the NmeCas9 cleavase comprises an amino acid sequence of any one of SEQ ID NO: 22, 107, 109, 120, 127, 136, or 137 or an amino acid sequence that is at least 85%, at least 90%, at least 95% identical to any one of SEQ ID NO: 22, 107, 109, 120, 127, 136, or 137.

In some embodiments, the nucleotide encoding the NmeCas9 cleavase comprises a nucleotide sequence of any one of SEQ ID NO: 21, 106, 108, 121-126, 128-133, 134, 135, 138, or 139; or a nucleotide sequence that is at least 85%, at least 90%, or at least 95% identical to any one of SEQ ID NO: SEQ ID NO: 21, 106, 108, 121-126, 128-133, 134, 135, 138, or 139.

In some embodiments, the NmeCas9 cleavase is a Nme2Cas9 comprises an amino acid sequence of any one of SEQ ID NO: 22, 109, or 136 or an amino acid sequence that is at least 85%, at least 90%, at least 95% identical to any one of SEQ ID NO: 22, 109, or 136; or (b) the nucleotide encoding the NmeCas9 cleavase comprises a nucleotide sequence of any one of SEQ ID NO: 21, 108, or 138; or a nucleotide sequence that is at least 85%, at least 90%, or at least 95% identical to any one of SEQ ID NO: 21, 108, or 138.

In some embodiments, the CjeCas9 cleavase comprises an amino acid sequence of SEQ ID NO: 144; or an amino acid sequence that is at least 85%, at least 90%, or at least 95% identical to SEQ ID NO: 144. In some embodiments, the nucleotide encoding the CjeCas9 cleavase comprises a sequence of SEQ ID NO: 143 or a nucleotide sequence that is at least 85%, at least 90%, or at least 95% identical to SEQ ID NO: 143.

In some embodiments, the SmuCas9 cleavase comprises an amino acid sequence of SEQ ID NO: 142; or an amino acid sequence that is at least 85%, at least 90%, or at least 95% identical to SEQ ID NO: 142. In some embodiments, the nucleotide encoding the SmuCas9 cleavase comprises an open reading frame (ORF) comprising a sequence of SEQ ID NO: 140 or 141 or a nucleotide sequence that is at least 85%, at least 90%, or at least 95% identical to SEQ ID NO: 140 or 141.

### C. Linkers

In some embodiments, the fusion protein described herein further comprises a linker between the first cleavase and the second cleavase. In some embodiments, the linker is an organic molecule, polymer, or chemical moiety. In some embodiments, the linker is a peptide linker. In some embodiments, the nucleic acid encoding the polypeptide comprising the first cleavase or the second cleavase further comprises a sequence encoding the peptide linker.

In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

In some embodiments, the peptide linker is any stretch of amino acids having at least 11, at least 21, at least 31, at least 41, at least 51, at least 61, at least 71, at least 81, or at least 91 amino acids.

In some embodiments, the fusion protein comprises a peptide linker between the first cleavase and the second cleavase. In some embodiments, the fusion protein comprises a peptide linker between the first cleavase and the second cleavase and the peptide linker comprises at least 10, at least 20, at least 30, at least 40, at least 50, at least 60, at least 70, at least 80 amino acids.

In some embodiments, the fusion protein comprises a peptide linker between the first cleavase and the second cleavase and the peptide linker comprises 11, 21, 31, 41, 51, 61, 71, or 81 amino acid residues.

In some embodiments, the fusion protein comprises a peptide linker between the first cleavase and the second cleavase and the peptide linker comprises an amino acid sequence of any one of SEQ ID NOs: 150-158; or an amino acid sequence is at least 85%, at least 90%, or at least 95% identical to any one of SEQ ID NOs: 150-158.

Other types of peptide linkers may be used herein. The peptide lunker may be the 16 residue “XTEN” linker, or a variant thereof (See, e.g., Schellenberger et al. A recombinant polypeptide extends the in vivo half-life of peptides and proteins in a tunable manner. Nat. Biotechnol. 27, 1186-1190 (2009)). In some embodiments, the XTEN linker comprises a sequence that is any one of SGSETPGTSESATPES (SEQ ID NO: 301), SGSETPGTSESA (SEQ ID NO: 302), or SGSETPGTSESATPEGGSGGS (SEQ ID NO: 303). In some embodiments, the XTEN linker consists of the sequence SGSETPGTSESATPES (SEQ ID NO: 301), SGSETPGTSESA (SEQ ID NO: 302), or SGSETPGTSESATPEGGSGGS (SEQ ID NO: 303).

In some embodiments, the peptide linker comprises a (GGGGS)ₙ (e.g., SEQ ID NOs: 305, 309-311, 314-318, 320-331, or 333-359), a (G)ₙ, an (EAAAK)ₙ (e.g., SEQ ID NOs: 306, 310-312, 315-318, 320-331, or 334-360), a (GGS)ₙ, an SGSETPGTSESATPES (SEQ ID NO: 301) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 150-158 and 301-365. In some embodiments, the peptide linker comprises a sequence of SEQ ID NO: 150. In some embodiments, the peptide linker comprises a sequence of SEQ ID NO: 151. In some embodiments, the peptide linker comprises a sequence of SEQ ID NO: 152. In some embodiments, the peptide linker comprises a sequence of SEQ ID NO: 153. In some embodiments, the peptide linker comprises a sequence of SEQ ID NO: 154. In some embodiments, the peptide linker comprises a sequence of SEQ ID NO: 155. In some embodiments, the peptide linker comprises a sequence of SEQ ID NO: 156. In some embodiments, the peptide linker comprises a sequence of SEQ ID NO: 157. In some embodiments, the peptide linker comprises a sequence of SEQ ID NO: 158. In some embodiments, the peptide linker comprises one or more sequences selected from SEQ ID NO: 301, SEQ ID NO: 302, SEQ ID NO: 303, SEQ ID NO: 361, SEQ ID NO: 362, SEQ ID NO: 363. SEQ ID NO: 364 and SEQ ID NO: 365. In some embodiments, the peptide linker comprises a sequence of SEQ ID NO: 361.

### D. Nuclear Localization Signals (NLS)

In some embodiments, the heterologous functional domain may facilitate transport of the fusion protein disclosed herein into the nucleus of a cell. For example, the heterologous functional domain may be a nuclear localization signal (NLS). In some embodiments, the fusion protein comprises a nuclear localization signal (NLS).

In some embodiments, the fusion protein may be fused with 1-10 NLS(s). In some embodiments, the fusion protein disclosed herein may be fused with 1-5 NLS(s). In some embodiments, the fusion protein may be fused with one NLS.

Where one NLS is used, the NLS may be fused at the N-terminus or the C-terminus of the fusion protein. In some embodiments, the fusion protein disclosed herein may be fused C-terminally to at least one NLS. An NLS may also be inserted within the fusion protein. In other embodiments, the fusion protein may be fused with more than one NLS.

In some embodiments, the fusion protein may be fused with 2, 3, 4, or 5 NLSs. In some embodiments, the fusion protein may be fused with two NLSs. In certain circumstances, the two NLSs may be the same (e.g., two SV40 NLSs) or different. In some embodiments, the fusion protein is fused to two SV40 NLS sequences at the C-terminus. In some embodiments, the fusion protein may be fused with two NLSs, one at the N-terminus and one at the C-terminus. In some embodiments, the fusion protein may be fused with 3 NLSs.

In some embodiments, the fusion protein may be fused with no NLS.

In some embodiments, the NLS may be a monopartite sequence, such as, e.g., the SV40 NLS, for example, PKKKRKVE (SEQ ID NO: 366), KKKRKVE (SEQ ID NO: 367), PKKKRKV (SEQ ID NO: 371) or PKKKRRV (SEQ ID NO: 383). In some embodiments, the NLS may be a bipartite sequence, such as the NLS of nucleoplasmin, KRPAATKKAGQAKKKK (SEQ ID NO: 384). In a specific embodiment, a single PKKKRKV (SEQ ID NO: 371) NLS may be fused at the C-terminus of the first cleavase or the second cleavase. One or more linkers are optionally included at the fusion site (e.g., between fusion protein disclosed herein and NLS).

In some embodiments, one or more NLS(s) according to any of the foregoing embodiments are present in the fusion protein in combination with one or more additional heterologous functional domains, such as any of the heterologous functional domains described below.

In some embodiments, the fusion protein comprises a nuclear localization signal (NLS) and the NLS is present at the C-terminus of the fusion protein. In some embodiments, the fusion protein comprises a nuclear localization signal (NLS) and the NLS is present at the N-terminus of the fusion protein. In some embodiments, the fusion protein comprises a nuclear localization signal (NLS) and the NLS is present at both the N-terminus and C-terminus of the fusion protein. In some embodiments, the fusion protein comprises a nuclear localization signal (NLS) and the NLS is present between the first or second Cas9 protein and the linker sequence. In some embodiments, the fusion protein comprises a nuclear localization signal (NLS) and the NLS is present within the linker sequence. In some embodiments, the fusion protein comprises a nuclear localization signal (NLS) and the NLS is present between C-terminus of the first cleavase disclosed herein and the linker sequence. In some embodiments, the fusion protein comprises a nuclear localization signal (NLS) and the NLS is present between the C-terminus of the second cleavase disclosed herein and the linker sequence. In some embodiments, the fusion protein comprises a nuclear localization signal (NLS) and the NLS is present between N-terminus of the first cleavase disclosed herein and the linker sequence. In some embodiments, the fusion protein comprises a nuclear localization signal (NLS) and the NLS is present between N-terminus of the second cleavase disclosed herein and the linker sequence.

In some embodiments, the fusion protein comprises a nuclear localization signal (NLS), and wherein the NLS comprises a sequence having at least 80%, at least 85%, at least 90%, or at least 95% identity to any one of SEQ ID NOs: 366-369 and 371-384. In some embodiments, the fusion protein comprises one, two, or three nuclear localization signals (NLSs) independently selected from SEQ ID NOs: 366-369 and 371-384.

In some embodiments, the fusion protein comprises a nuclear localization signal (NLS), and wherein the NLS is encoded by a sequence having at least 80%, at least 85%, at least 90%, at least 95%, at least 98% or 100% identity to the sequence of any one of SEQ ID NOs: 370 and 385-397.

### E. Exemplary Cas9-Cas9 Fusion Protein

In some embodiments, the fusion protein comprises, from N-terminus to C-terminus: the first cleavase; a peptide linker, optionally wherein the linker comprises 81 amino acid residues; the second cleavase; and an NLS comprising an SV40 NLS.

In some embodiments, the fusion protein comprises, from N-terminus to C-terminus: a first NLS, wherein the first NLS comprises an SV40 NLS; the second cleavase; a peptide linker, optionally wherein the peptide linker comprises 41 amino acids; the first cleavase; a second NLS, wherein the second NLS comprising an SV40 NLS.

In some embodiments, the fusion protein comprises, from N-terminus to C-terminus: the second cleavase; a peptide linker, optionally wherein the peptide linker comprises 41 amino acids; the first cleavase; and an NLS, optionally wherein the NLS comprises an SV40 NLS. In some embodiments, the first cleavase comprises a SpyCas9. In some embodiments, the second cleavase comprises a CjeCas9, an SmuCas9, an Nme1Cas9, an Nme2Cas9, or an Nme3 Cas9.

In some embodiments, a fusion protein disclosed herein comprising (a) a first cleavase and a guide RNA (gRNA) that targets at least one genomic locus and that is cognate to the first cleavase; and (b) a second cleavase and a gRNA that targets at least one genomic locus and that is cognate to the second cleavase, wherein the first cleavase is orthogonal to the second cleavase is provided. In some embodiments, the first cleavase comprises a SpyCas9. In some embodiments, the second cleavase comprises a CjeCas9, an SmuCas9, an Nme1Cas9, an Nme2Cas9, or an Nme3 Cas9.

In some embodiments, a fusion protein disclosed herein comprising (a) a first cleavase and a guide RNA (gRNA) that targets at least one genomic locus and that is cognate to the first cleavase; and (b) a second cleavase and a gRNA that targets at least one genomic locus and that is cognate to the second cleavase, wherein the first cleavase is orthogonal to the second cleavase is provided. In some embodiments, the first cleavase comprises an R1333K SpyCas9. In some embodiments, the second cleavase comprises a CjeCas9, an SmuCas9, an Nme1Cas9, an Nme2Cas9, or an Nme3Cas9.

In some embodiments, a fusion protein disclosed herein comprising (a) a first cleavase and a guide RNA (gRNA) that targets at least one genomic locus and that is cognate to the first cleavase; and (b) a second cleavase and a gRNA that targets at least one genomic locus and that is cognate to the second cleavase, wherein the first cleavase is orthogonal to the second cleavase is provided. In some embodiments, the fusion protein disclosed herein comprises an R1333K SpyCas9 and a CjeCas9. In some embodiments, the fusion protein disclosed herein comprises an R1333K SpyCas9 and a SmuCas9. In some embodiments, the fusion protein disclosed herein comprises an R1333K Spy Cas9 and an Nme1Cas9. In some embodiments, the fusion protein disclosed herein comprises an R1333K Spy Cas9 and an Nme2Cas9. In some embodiments, the fusion protein disclosed herein comprises an R1333K Spy Cas9 and an Nme3Cas9.

In some embodiments, the first cleavase and the second cleavase are connected via a linker. In some embodiments, the first cleavase and the second cleavase are connected via a peptide linker. In some embodiments, the fusion protein disclosed herein further comprises one or more additional heterologous functional domains. In some embodiments, the first cleavase further comprises one or more nuclear localization sequences (NLSs) (described herein) at the C-terminal of the polypeptide or the N-terminal of the polypeptide. In some embodiments, the one or more NLS comprises one or more sequences selected from SEQ ID NOs: 366-384.

In some embodiments, a fusion protein disclosed herein comprising (a) a first cleavase and a guide RNA (gRNA) that targets at least one genomic locus and that is cognate to the first cleavase; and (b) a second cleavase and a gRNA that targets at least one genomic locus and that is cognate to the second cleavase, wherein the first cleavase is orthogonal to the second cleavase is provided. In some embodiments, the first cleavase comprises R1333K SpyCas9 and the second cleavase comprises Nme2 Cas9. In some embodiments, the R1333K SpyCas9 and the Nme2 Cas9 are fused via a linker. In some embodiments, the first cleavase comprises R1333K SpyCas9 and the second cleavase comprises Cje Cas9. In some embodiments, the R1333K SpyCas9 and the Cje Cas9 are fused via a linker. In some embodiments, the first cleavase comprises R1333K SpyCas9 and the second cleavase comprises Smu Cas9. In some embodiments, the R1333K SpyCas9 and the Smu Cas9 are fused via a linker. In some embodiments, the fusion protein disclosed herein comprises an NLS at the C-terminal of the polypeptide. In some embodiments, the fusion protein disclosed herein comprises a first NLS at the C-terminal of the polypeptide and a second NLS at the N-terminal of the polypeptide. In some embodiments, the fusion protein disclosed herein comprises a first and second NLS at the C-terminal of the polypeptide and a third NLS at the N-terminal of the polypeptide. In some embodiments, the fusion protein comprises a nuclear localization signal (NLS) and the NLS is present between the first or second Cas9 protein and the linker sequence. In some embodiments, the fusion protein comprises a nuclear localization signal (NLS) and the NLS is present within the linker sequence. In some embodiments, the fusion protein comprises a nuclear localization signal (NLS) and the NLS is present within the linker sequence. In some embodiments, the fusion protein comprises a nuclear localization signal (NLS) and the NLS is present between C-terminus of the first cleavase disclosed herein and the linker sequence. In some embodiments, the fusion protein comprises a nuclear localization signal (NLS) and the NLS is present between the C-terminus of the second cleavase disclosed herein and the linker sequence. In some embodiments, the fusion protein comprises a nuclear localization signal (NLS) and the NLS is present between N-terminus of the first cleavase disclosed herein and the linker sequence. In some embodiments, the fusion protein comprises a nuclear localization signal (NLS) and the NLS is present between N-terminus of the second cleavase disclosed herein and the linker sequence.

In some embodiments, the fusion protein disclosed herein comprises an amino acid sequence of SEQ ID NOs: 3, 5, 7, 10, 13, 16, 40, 43, 45, 47, 50, 52, 55, 57, 60, 62, 65, 67, 70, 72, 75, 77, 80, 82, 85, 87, 90, 92, 101, or 105, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, 13, 16, 40, 43, 45, 47, 50, 52, 55, 57, 60, 62, 65, 67, 70, 72, 75, 77, 80, 82, 85, 87, 90, 92, 101, or 105.

In some embodiments, the nucleic acid encoding the fusion protein disclosed herein comprises a nucleotide sequence of SEQ ID NOs: 1, 2, 4, 6-8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104.

In some embodiments, the fusion protein disclosed herein comprises an amino acid sequence of SEQ ID NOs: 3, 5, 7, 10, or 13, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, or 13.

In some embodiments, the nucleic acid encoding the fusion protein disclosed herein comprises a nucleotide sequence of SEQ ID NOs: 1, 2, 4, 6, 8, 9, 11 or 12, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11 or 12.

In some embodiments, the fusion protein disclosed herein comprises an amino acid sequence of SEQ ID NOs: 5, 7, 10, or 13 or an amino acid sequence that is at least 85%, at least 90%, or at least 95%% identical to an amino acid sequence of SEQ ID NOs: 5, 7, 10, or 13.

or In some embodiments, the nucleic acid encoding the fusion protein disclosed herein comprises a nucleotide sequence of SEQ ID NOs: 4, 6, 8, 9, 11 or 12, a nucleotide sequence that is at least 85%, at least 90%, or at least 95% identical to SEQ ID NO: 4, 6, 8, 9, 11 or 12,

### III. Exemplary Composition and Methods for Genomic Editing

Compositions comprising the Cas9-Cas9 fusion protein or a nucleic acid (e.g., mRNA) encoding the fusion protein and the guide RNAs are provided. In some embodiments, the composition comprises (a) a polynucleotide comprising an open reading frame (ORF) encoding a fusion protein, wherein the fusion protein comprises a first cleavase and a second cleavase; (b) a first guide RNA that directs the first cleavase to a first genomic locus; and (c) a second guide RNA that directs the second cleavase to a second genomic locus, wherein the second genomic locus is different from the first genomic locus. In some embodiments, the first cleavase is a S. pyogenes (Spy)Cas9 cleavase comprising a R1333K mutation within its PAM recognition domain. In some embodiments, the second cleavase is a N. meningitidis (Nme)Cas9 cleavase (e.g., an Nme1Cas9, an Nme2Cas9, or an Nme3 Cas9), a C. jejuni (Cje) Cas9 cleavase, or a Simonsiella muelleri (Smu) Cas9. The compositions disclosed herein may be used for producing a modification in the genome of a target cell.

Methods of producing a modification in the genome of a target cell are provided. In some embodiments, provided herein is a method comprising contacting the cell with an orthogonal Cas9-Cas9 fusion protein comprising a first cleavase and a second cleavase, or a nucleic acid encoding the fusion protein, thereby excising a DNA sequence between a first cleavage site cleaved by the first Cas9 cleavase and a second cleavage site cleaved by the second Cas9 cleavase. In some embodiments, the method comprises contacting the cell with: (a) a fusion protein, or a nucleic acid encoding a fusion protein, wherein the fusion protein comprises a first cleavase and a second cleavase; and (b) a first guide RNA that directs the first cleavase to a first genomic locus; and (c) a second guide RNA that directs the second cleavase to a second genomic locus, wherein the second genomic locus is different from the first genomic locus.

Method of producing an edited (or engineered) cell or a population of engineered cells are also contemplated. In some embodiments, the method comprises a modification in the genome of the target cell or cells, the method comprising contacting the cell or cells with: (a) a fusion protein, or a nucleic acid encoding a fusion protein, wherein the fusion protein comprises a first cleavase and a second cleavase; and (b) a first guide RNA that directs the first cleavase to a first genomic locus; and (c) a second guide RNA that directs the second cleavase to a second genomic locus, wherein the second genomic locus is different from the first genomic locus.

In some embodiments, the first guide RNA and the second guide RNA target two non-overlapping genomic loci. In some embodiments, the two non-overlapping genomic loci are separated by equal to or less than 500, 450, 400, 350, 300, 250, 200, 150, 140, 130, 120, 110, 100, 90, 80, 70, 60, 50, 40, 30, or 25 nucleotides. In some embodiments, the two non-overlapping genomic loci are separated by equal to or less than 150, 140, 130, 120, 110, 100, 90, 80, 70, 60, 50, 40, 30, or 25 nucleotides. In some embodiments, the two non-overlapping genomic loci are separated by 25-150, 30-150, 40-150, 50-150, 60-150, 70-150, 80-150, 90-150, 100-150, 110-150, 120-150, 130-150, or 140-150 nucleotides. In some embodiments, the two non-overlapping genomic loci are separated by 25-110, 30-110, 40-110, 50-110, 60-110, 70-110, 80-110, 90-110, or 100-110 nucleotides. In some embodiments, the two non-overlapping genomic loci are separated by 25-90, 30-90, 40-90, 50-90, 60-90, 70-90, or 80-90 nucleotides. In some embodiments, the two non-overlapping genomic loci are separated by equal to or less than 110 nucleotides. In some embodiments, the first guide RNA is a single guide RNA (sgRNA). In some embodiments, the first guide RNA is a SpyCas9 guide RNA. In some embodiments, the second guide RNA is a NmeCas9 guide RNA. In some embodiments, one or both of the guide RNAs comprises one or more mismatches to the target sequences.

In some embodiments, the nucleic acid encoding the fusion protein are delivered to the cell on at least one vector. In some embodiments, the fusion protein or the nucleic acid encoding the fusion protein are delivered to the cell via electroporation. In some embodiments, the first guide RNA is delivered to the cell via electroporation. In some embodiments, the second guide RNA is delivered to the cell via electroporation.

In some embodiments, one or more of the nucleic acids encoding the fusion protein, the first guide RNA, and the second guide RNA are associated with one or more lipid nanoparticle (LNP). In some embodiments, the nucleic acids each encoding the fusion protein, the first guide RNA, and the second guide RNA are each associated with a separate lipid nanoparticle (LNP). In some embodiments, the first guide RNA and the second guide RNA are associated with a same lipid nanoparticle (LNP). In some embodiments, all of the nucleic acid encoding the fusion protein, the first guide RNA, and the second guide RNA are associated with a same lipid nanoparticle (LNP). Delivery of the polynucleotides and compositions via LNPs are further described below.

In some embodiments, the modification is in vivo. In some embodiments, the modification is ex vivo.

In some embodiments, the modification comprises a deletion of equal to or less than 500, 450, 400, 350, 300, 250, 200, 150, 140, 130, 120, 110, 100, 90, 80, 70, 60, 50,40,30 nucleotides. In some embodiments, the modification comprises a deletion of equal to or less than 150, 140, 130, 120, 110, 100, 90, 80, 70, 60, 50, 40, 30 nucleotides. In some embodiments, the modification comprises a deletion of 25-150, 30-150, 40-150, 50-150, 60-150, 70-150, 80-150, 90-150, 100-150, 110-150, 120-150, 130-150, or 140-150 nucleotides. In some embodiments, the modification comprises a deletion of 25-110, 30-110, 40-110, 50-110, 60-110, 70-110, 80-110, 90-110, or 100-110 nucleotides. In some embodiments, the modification comprises a deletion of 25-90, 30-90, 40-90, 50-90, 60-90, 70-90, or 80-90 nucleotides.

In some embodiments, the modification comprises a deletion of equal to or less than 500, 450, 400, 350, 300, 250, 200, 150, 140, 130, 120, 110, 100, 90, 80, 70, 60, 50, 40, 30, or 25 contiguous nucleotides. In some embodiments, the modification comprises a deletion of equal to or less than 500-450, 500-400, 500-350, 500-300, 500-250, 500-200, 500-150, 500-140, 500-130, 500-120, 500-110, 500-100, 500-90, 500-80, 500-70, 500-60, 500-50, 500-40, or 500-30 contiguous nucleotides. In some embodiments, the modification comprises a deletion of equal to or less than 150, 140, 130, 120, 110, 100, 90, 80, 70, 60, 50, 40, 30, or 25 contiguous nucleotides.

In some embodiments, the modification comprises a deletion of 25-150, 30-150, 40-150, 50-150, 60-150, 70-150, 80-150, 90-150, 100-150, 110-150, 120-150, 130-150, or 140-150 contiguous nucleotides. In some embodiments, the modification comprises a deletion of 25-110, 30-110, 40-110, 50-110, 60-110, 70-110, 80-110, 90-110, or 100-110 contiguous nucleotides. In some embodiments, the modification comprises a deletion of 25-90, 30-90, 40-90, 50-90, 60-90, 70-90, or 80-90 contiguous nucleotides.

In some embodiments, the modification comprises a deletion of equal to or larger than 25, 35, 45, 55, 65, 75, 85, 95, 100, 105, 115, 124, 135, 145, 155, 205, 255, 305, 355, 405, or 455 nucleotides. In some embodiments, the modification comprises a deletion of equal to or larger than about 25, about 35, about 45, about 55, about 65, about 75, about 85, about 95, about 100, or about 105 nucleotides. In some embodiments, the modification comprises a deletion of equal to or larger than 25, 35, 45, 55, 65, 75, 85, 95, 100, 105, 115, 124, 135, 145, 155, 205, 255, 305, 355, 405, or 455 contiguous nucleotides. In some embodiments, the modification comprises a deletion of equal to or larger than about 25, about 35, about 45, about 55, about 65, about 75, about 85, about 95, about 100, or about 105 contiguous nucleotides.

In some embodiments, the modification comprises a deletion of each of the nucleotides between a first cleavage site and a second cleavage site. In some embodiments, the deletion comprises one or both protospacer adjacent motif (PAM) sites recognized by the first cleavase or the second cleavase.

In some embodiments, the modification reduces or eliminates the expression of one or more mRNAs or proteins.

In some embodiments, the modification reduces or eliminates the expression of one or more mRNAs by at least 50%, at least 55%, at least 60%, at least 65%, at least 70%, at least 75%, at least 80%, at least 85%, at least 90%, or at least 95%. In some embodiments, the modification reduces or eliminates the expression of one or more proteins by at least 50%, at least 55%, at least 60%, at least 65%, at least 70%, at least 75%, at least 80%, at least 85%, at least 90%, or at least 95%.

In some embodiments, the modification increases the expression of one or more RNAs or proteins. In some embodiments, the modification increases the expression of one or more mRNAs by at least two-fold, at least three-fold, or at least four-fold or at least five-fold. In some embodiments, the modification increases the expression of one or more proteins by at least two-fold, at least three-fold, or at least four-fold or at least five-fold.

In some embodiments, the expression of one or more target protein is reduced or eliminated in a population of cells using the methods and compositions disclosed herein. In some embodiments, the population of cells is at least 55%, 60%, 65%, 70%, 80%, 90%, 91%, 92%, 93%, 94%, 95%, 96%, 97%, 98%, or 99% negative for a target protein as measured by flow cytometry relative to a population of unmodified cells.

In some embodiments, the expression of one or more target protein is increased in a population of cells using the methods and compositions disclosed herein. In some embodiments, the population of cells is at least 55%, 60%, 65%, 70%, 80%, 90%, 91%, 92%, 93%, 94%, 95%, 96%, 97%, 98%, or 99% positive for a target protein as measured by flow cytometry relative to a population of unmodified cells.

In some embodiments, the modification results in the deletion of a start codon. In some embodiments, the modification results in the deletion of a splice site. In some embodiments, the modification results in the deletion of a splicing enhancer. In some embodiments, the modification results in the deletion of a splicing repressor. In some embodiments, the modification results in the deletion of a transcription factor binding site.

### A. Fusion Protein Via Intein

In some embodiments, the composition disclosed herein include (a) a first polynucleotide comprising an ORF encoding the first polypeptide, wherein the first polypeptide comprises first cleavase and a first intein and (b) a second polynucleotide comprising an ORF encoding the second polypeptide, wherein the first polypeptide binds to the second polypeptide through intein catalysis.

In some embodiments, a method of producing a modification in the genome of a target cell is provided. The method comprises (a) contacting the cell with a first polypeptide, or a nucleic acid encoding the first polypeptide; (b) contacting the cell with a second polypeptide, or a nucleic acid encoding the first polypeptide, wherein the second polypeptide comprises a second cleavase and a second intein capable of binding the first intein; and wherein the first polypeptide binds to the second polypeptide through intein catalysis, (b) contacting the cell with a first guide RNA that directs the first cleavase to a first genomic locus; and (c) contacting the cell with a second guide RNA that directs the second cleavase to a second genomic locus, wherein the second genomic locus is different from the first genomic locus.

In some embodiments, the first polypeptide comprises, from N-terminus to C-terminus: the first intein; the first cleavase is a S. pyogenes (Spy)Cas9 cleavase, said SpyCas9 cleavase comprising a R1333K mutation within its protospacer adjacent motif recognition domain; and a first NLS comprising an SV40 NLS.

In some embodiments, the second polypeptide comprises, from N-terminus to C-terminus: a second NLS comprising an SV40 NLS; a third NLS comprising a nucleoplasmin NLS; the second cleavase; a peptide linker, optionally wherein the peptide linker comprises 41 or 81 amino acid residues; and the second intein capable of binding the first intein.

In some embodiments, the first polypeptide comprises an amino acid sequence of SEQ ID NOs: 28 or 31 or an amino acid sequence that is at least 85%, at least 90%, or at least 95% identical to SEQ ID NO: 28 or 31.

In some embodiments, the nucleic acid or nucleic acids encoding the first polypeptide or polypeptides comprises a sequence of SEQ ID NOs: 27 or 30, or a nucleotide sequence that is at least 85%, at least 90%, or at least 95% identical to SEQ ID NOs: 27 or 30.

In some embodiments, the second polypeptide comprises an amino acid sequence of SEQ ID NOs: 25 or 34 or an amino acid sequence that is at least 85%, at least 90%, or at least 95% identical to SEQ ID NO: 25 or 34. In some embodiments, the nucleic acid or nucleic acids encoding the polypeptide or polypeptides comprises a sequence of SEQ ID NOs: 24 or 33, or a nucleotide sequence that is at least 85%, at least 90%, or at least 95% identical to SEQ ID NOs: 24 or 33.

### IV. Additional Features

The following section provides additional features of the Cas9 cleavases, fusion protein, the nucleic acid or nucleic acids encoding the same, guide RNAs, and compositions disclosed herein. In any of the embodiments set forth herein, the nucleic acid or nucleic acids may be one or more expressions construct comprising a promoter operably linked to an ORF encoding Cas9 cleavase or more polypeptides comprising the fusion protein disclosed herein.

### A. Codon-Optimization

In some embodiments, the nucleic acid or nucleic acids encoding polypeptide or polypeptides comprising the first cleavase, second cleavase or fusion protein disclosed herein comprises one or more ORFs comprising one or more codon optimized nucleic acid sequences. In some embodiment, the codon optimized nucleic acid sequence or sequences comprise minimal adenine codons and/or minimal uridine codons.

A given ORF can be reduced in adenine content or adenine dinucleotide content, for example, by using minimal adenine codons in a sufficient fraction of the ORF. For example, one or more amino acid sequence for the first cleavase, second cleavase or fusion protein disclosed herein described herein can be back-translated into one or more ORF sequences by converting amino acids to codons, wherein some or all of the ORF or ORFs uses the exemplary minimal adenine codons shown below. In some embodiments, at least about 50%, at least about 55%, at least about 60%, at least about 65%, at least about 70%, at least about 75%, at least about 80%, at least about 85%, at least about 90%, at least about 95%, at least about 98%, at least about 99%, or 100% of the codons in the ORF are codons listed in Table 1A.

TABLE 1A
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, the ORF or ORFs may consist of a set of codons of which at least about 75%, at least about 80%, at least about 85%, at least about 90%, at least about 95%, at least about 98%, at least about 99%, or 100% of the codons are codons listed in Table 2.

To the extent feasible, any of the features described above with respect to low adenine content can be combined with any of the features described above with respect to low uridine content. So too for uridine and adenine dinucleotides. Similarly, the content of uridine nucleotides and adenine dinucleotides in the ORF or ORFs may be as set forth above. Similarly, the content of uridine dinucleotides and adenine nucleotides in the ORF or ORFs may be as set forth above.

In some instances, a given ORF can be reduced in uridine content or uridine dinucleotide content, for example, by using minimal uridine codons in a sufficient fraction of the ORF. In other instances, a given ORF can be reduced in uridine and adenine nucleotide or dinucleotide content, for example, by using minimal uridine and adenine codons in a sufficient fraction of the ORF. For example, one or more amino acid sequence for the first cleavase, second cleavase or fusion protein disclosed herein can be back-translated into an ORF sequence by converting amino acids to codons, wherein some or all of the ORF or ORFs uses the minimal uridine codons or minimal uridine and adenine codons. Exemplary the exemplary minimal uridine codons or exemplary minimal uridine and adenine codons may be found in WO

In some embodiments, the ORF may have codons that increase translation in a mammal, such as a human. In further embodiments, ORF is an mRNA and comprises codons that increase translation in an organ, such as the liver, of the mammal, e.g., a human. In further embodiments, the ORF may have codons that increase translation in a cell type, such as a hepatocyte, of the mammal, e.g., a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level. In some embodiments, at least about 75%, at least about 80%, at least about 85%, at least about 90%, at least about 95%, at least about 96%, at least about 97%, at least about 98%, at least about 99%, or 100% of the codons in an ORF are codons corresponding to highly expressed tRNAs (e.g., the highest-expressed tRNA for each amino acid) in a mammal, such as a human. In some embodiments, at least 75%, at least 80%, at least 85%, at least 90%, at least 95%, at least 96%, at least 97%, at least 98%, at least 99%, or 100% of the codons in an ORF are codons corresponding to highly expressed tRNAs (e.g., the highest-expressed tRNA for each amino acid) in a mammalian organ, such as a human organ. Alternatively, codons corresponding to highly expressed tRNAs in an organism (e.g., human) in general may be used.

Any of the foregoing approaches to codon selection can be combined with the minimal uridine or adenine codons e.g., by starting with the codons of Table 1 and then where more than one option is available, using the codon that corresponds to a more highly-expressed tRNA, either in the organism (e.g., human) in general, or in an organ or cell type of interest (e.g., human liver or human hepatocytes).

In some embodiments, at least 75%, at least 80%, at least 85%, at least 90%, at least 95%, at least 96%, at least 97%, at least 98%, at least 99%, or 100% of the codons in an ORF are codons from a codon set shown in Table 1B (e.g., the low U 1, low A, or low A/U codon set). The codons in the low U 1, low G, low A, and low A/U sets use codons that minimize the indicated nucleotides while also using codons corresponding to highly expressed tRNAs where more than one option is available. In some embodiments, at least 75%, at least 80%, at least 85%, at least 90%, at least 95%, at least 96%, at least 97%, at least 98%, at least 99%, or 100% of the codons in an ORF are codons from the low U 1 codon set shown in Table 1B. In some embodiments, at least 75%, at least 80%, at least 85%, at least 90%, at least 95%, at least 96%, at least 97%, at least 98%, at least 99%, or 100% of the codons in an ORF are codons from the low A codon set shown in Table 1B. In some embodiments, at least 75%, at least 80%, at least 85%, at least 90%, at least 95%, at least 96%, at least 97%, at least 98%, at least 99%, or 100% of the codons in an ORF are codons from the low A/U codon set shown in Table 1B.

TABLE 1B
Exemplary Codon Sets.

| Amino Acid | Low U 1 | Low U 2 | Low A | Low A/U |
|---|---|---|---|---|
| Gly | GGC | GGG | GGC | GGC |
| Glu | GAG | GAA | GAG | GAG |
| Asp | GAC | GAC | GAC | GAC |
| Val | GTG | GTA | GTG | GTG |
| Ala | GCC | GCG | GCC | GCC |
| Arg | AGA | CGA | CGG | CGG |
| Ser | AGC | AGC | TCC | AGC |
| Lys | AAG | AAA | AAG | AAG |
| Asn | AAC | AAC | AAC | AAC |
| Met | ATG | ATG | ATG | ATG |
| Ile | ATC | ATA | ATC | ATC |
| Thr | ACC | ACG | ACC | ACC |
| Trp | TGG | TGG | TGG | TGG |
| Cys | TGC | TGC | TGC | TGC |
| Tyr | TAC | TAC | TAC | TAC |
| Leu | CTG | CTA | CTG | CTG |
| Phe | TTC | TTC | TTC | TTC |
| Gln | CAG | CAA | CAG | CAG |
| His | CAC | CAC | CAC | CAC |

### B. Heterologous Functional Domains

In some embodiments, the heterologous functional domain may be capable of modifying the intracellular half-life of the fusion protein disclosed herein. In some embodiments, the half-life of the fusion protein disclosed herein may be increased. In some embodiments, the half-life of the fusion protein disclosed herein may be reduced. In some embodiments, the heterologous functional domain may be capable of increasing the stability of the fusion protein disclosed herein. In some embodiments, the heterologous functional domain may be capable of reducing the stability of the fusion protein disclosed herein. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the polypeptide may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Any known fluorescent proteins may be used as the marker domain such as GFP, YFP, EBFP, ECFP, DsRed or any other suitable fluorescent protein. In some embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. In some embodiments, the marker domain may be a reporter gene. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the fusion protein disclosed herein to a specific organelle, cell type, tissue, or organ. In some embodiments, the heterologous functional domain may target the fusion protein disclosed herein to mitochondria.

### C. UTRs; Kozak Sequences

In some embodiments, the nucleic acid (e.g., mRNA) disclosed herein comprises a 5′ UTR, 3′ UTR, or 5′ and 3′ UTRs from Hydroxysteroid 17-Beta Dehydrogenase 4 (HSD17B4 or HSD) or globin such as human alpha globin (HBA), human beta globin (HBB), Xenopus laevis beta globin (XBG), bovine growth hormone, cytomegalovirus (CMV), mouse Hba-al, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the nucleic acid described herein does not comprise a 5′ UTR, e.g., there are no additional nucleotides between the 5′ cap and the start codon. In some embodiments, the nucleic acid comprises a Kozak sequence (described below) between the 5′ cap and the start codon, but does not have any additional 5′ UTR. In some embodiments, the nucleic acid does not comprise a 3′ UTR, e.g., there are no additional nucleotides between the stop codon and the poly-A tail.

In some embodiments, the polynucleotide comprises a 5′ UTR with at least 85%, at least 90%, or at least 95% identity to any one of SEQ ID NOs: 398-405. In some embodiments, the polynucleotide comprises a 3′ UTR with at least 85%, at least 90%, or at least 95% identity to any one of SEQ ID NOs: 406-413. In some embodiments, the polynucleotide comprises a 5′ UTR and 3′ UTR from the same source.

In some embodiments, the nucleic acid herein comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN (SEQ ID NO: 417) wherein at least one of the following is true: the first N is A or G and the second N is G. In the context of a nucleotide sequence, R means a purine (A or G). In some embodiments, the Kozak sequence is RNNRUGN (SEQ ID NO: 418), NNNRUGG (SEQ ID NO: 419), RNNRUGG (SEQ ID NO: 420), RNNAUGN (SEQ ID NO: 421), NNNAUGG (SEQ ID NO: 422), RNNAUGG (SEQ ID NO: 423), or GCCACCAUG (SEQ ID NO: 424).

### D. Poly-A Tail

In some embodiments, the nucleic acid disclosed herein further comprises a polyadenylated (poly-A) tail. The poly-A tails may comprise at least 8 consecutive adenine nucleotides, but also comprise one or more non-adenine nucleotide. As used herein, “non-adenine nucleotides” refers to any natural or non-natural nucleotides that do not comprise adenine. Guanine, thymine, and cytosine nucleotides are exemplary non-adenine nucleotides. Thus, the poly-A tails on the nucleic acid described herein may comprise consecutive adenine nucleotides located 3′ to nucleotides encoding a polypeptide of interest. In some instances, the poly-A tails on the nucleic acid comprise non-consecutive adenine nucleotides located 3′ to nucleotides encoding the polypeptide, wherein non-adenine nucleotides interrupt the adenine nucleotides at regular or irregularly spaced intervals.

In some embodiments, the poly-A tail is encoded in a plasmid used for in vitro transcription of an mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, at least 9, at least 10, at least 11, or at least 12 consecutive adenine nucleotides. In some embodiments, the one or more non-adenine nucleotide is located after 8-50 consecutive adenine nucleotides. In some embodiments, the one or more non-adenine nucleotide is located after 8-100 consecutive adenine nucleotides.

In some embodiments, the poly-A tail comprises or contains one non-adenine nucleotide or one consecutive stretch of 2-10 non-adenine nucleotides.

In some embodiments, the non-adenine nucleotide is guanine, cytosine, or thymine. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides.

### E. Modified Nucleotides

In some embodiments, the nucleic acid disclosed herein comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof.

In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of the uridine positions in the nucleic acid disclosed herein are modified uridines. In some embodiments, 10%-25%, 15-25%, 25-35%, 35-45%, 45-55%, 55-65%, 65-75%, 75-85%, 85-95%, or 90-100% of the uridine positions in an mRNA disclosed herein are modified uridines, e.g., 5-methoxyuridine, 5-iodouridine, N1-methyl pseudouridine, pseudouridine, or a combination thereof. In some embodiments, 80-95% or 80-100% of the uridine positions in an mRNA disclosed herein are modified uridines, e.g., 5-methoxyuridine, 5-iodouridine, N1-methyl pseudouridine, pseudouridine, or a combination thereof.

In some embodiments, at least 10% of the uridine is substituted with a modified uridine. In some embodiments, 15% to 45% of the uridine is substituted with the modified uridine. In some embodiments, at least 20%, at least 30%, at least 40%, at least 50%, at least 60%, at least 70%, at least 80%, at least 90%, or 100% of the uridine is substituted with the modified uridine.

In some embodiments, at least 85% of the uridine is substituted with modified uridine.

In some embodiments, the modified uridine is one or more of N1-methyl-pseudouridine, pseudouridine or 5-iodouridine. In some embodiments, the modified uridine is N1-methyl-pseudouridine. In some embodiments, the modified uridine is pseudouridine. In some embodiments, the modified uridine is 5-iodouridine. In some embodiments, at least 85% of the uridine is substituted with the modified uridine. In some embodiments, 100% uridine is substituted with the modified uridine.

### F. 5′ Cap

In some embodiments, the nucleic acid disclosed herein comprises a 5′ cap, such as a Cap0, Cap1, or Cap2. A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the nucleic acid comprise a 2′-methoxy and a 2′-hydroxyl, respectively. In Cap2, the riboses of the first and second cap-proximal nucleotides of the nucleic acid both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic nucleic acids, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acids with a cap other than Cap1 or Cap2, potentially inhibiting translation of the nucleic acid.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap or a Cap0-like cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

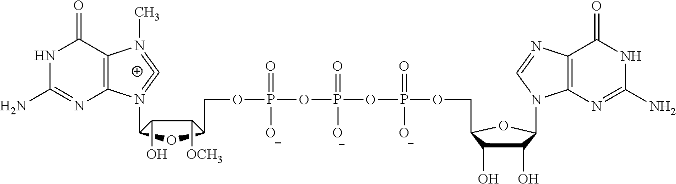

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

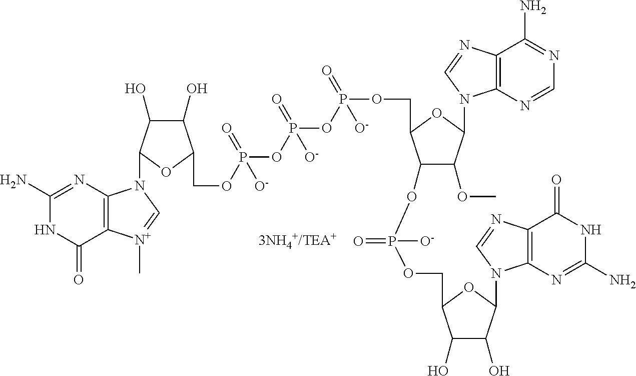 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21 ; "3NH4+/TEA+"]

Alternatively a cap can be added to an RNA post-transcriptionally. For example, Vaccinia capping enzyme is commercially available (New England Biolabs Cat. No. M2080S) and has RNA triphosphatase and guanylyltransferase activities, provided by its D subunit, and guanine methyltransferase, provided by its D12 subunit. As such, it can add a 7-methylguanine to an RNA, so as to give Cap0, in the presence of g-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### V. Guide RNAs

In some embodiments, the fusion protein disclosed herein comprises (a) a first cleavase and a guide RNA (gRNA) that targets a first genomic locus and that is cognate to the first cleavase; and (b) a second cleavase and a gRNA that targets a second genomic locus and that is cognate to the second cleavase, wherein the first genomic locus is different from the second genomic locus.

In some embodiments, the first gRNA that is cognate to the first cleavase is non-cognate to the second cleavase. In some embodiments, the second gRNA that is cognate to the second cleavase is non-cognate to the first cleavase.

### A. Target Sequences and Genes

In some embodiments, the methods and compositions of the present disclosure utilize an orthogonal Cas9-Cas9 fusion system directed by guide RNAs to excise a sequence between a first cleavage site cleaved by the first Cas9 cleavase and a second cleavage site cleaved by the second Cas9 cleavase.

For example, a target sequence may be recognized and cleaved by a Cas nuclease. In some embodiments, a target sequence for a Cas nuclease is located near the nuclease's cognate PAM sequence. In some embodiments, a Class 2 Cas nuclease may be directed by a gRNA to a target sequence of a gene, where the gRNA hybridizes with and the Class 2 Cas protein cleaves the target sequence. In some embodiments, the guide RNA hybridizes with and a Class 2 Cas nuclease cleaves the target sequence adjacent to or comprising its cognate PAM. In some embodiments, the target sequence may be complementary to a targeting sequence of the guide RNA. In some embodiments, the degree of complementarity between a targeting sequence of a guide RNA and the portion of the corresponding target sequence that hybridizes to the guide RNA may be about 80%, about 85%, about 90%, about 95%, about 97%, about 98%, about 99%, or 100%. In some embodiments, the percent identity between a targeting sequence of a guide RNA and the portion of the corresponding target sequence that hybridizes to the guide RNA may be about 80%, about 85%, about 90%, about 95%, about 97%, about 98%, about 99%, or 100%. In some embodiments, the homology region of the target is adjacent to a cognate PAM sequence. In some embodiments, the target sequence may comprise a sequence 100% complementary with the targeting sequence of the guide RNA. In other embodiments, the target sequence may comprise at least one mismatch, deletion, or insertion, as compared to the targeting sequence of the guide RNA.

The length of the target sequence may depend on the nuclease system used. For example, the targeting sequence of a guide RNA for a CRISPR/Cas system may comprise 10, 11, 12, 13, 14, 15, 16, 17, 18, 19, 20, 21, 22, 23, 24, 25, 26, 27, 28, 29, or 30 in length and the target sequence is a corresponding length, optionally adjacent to a PAM sequence. In some embodiments, the target sequence may comprise 15-24 nucleotides in length. In some embodiments, the target sequence may comprise 17-21 nucleotides in length. In some embodiments, the target sequence may comprise 20 nucleotides in length. In some embodiments, the target sequence may comprise 24 nucleotides in length. In some embodiments, the target sequence may comprise a part of target sequences recognized by one or more Cas nucleases.

The target nucleic acid molecule may be any DNA or RNA molecule that is endogenous or exogenous to a cell. In some embodiments, the target nucleic acid molecule may be an episomal DNA, a plasmid, a genomic DNA, viral genome, or chromosomal DNA. In some embodiments, the target sequence of the gene may be a genomic sequence from a cell or in a cell, including a human cell.

In further embodiments, the target sequence may be a viral sequence. In further embodiments, the target sequence may be a pathogen sequence. In yet other embodiments, the target sequence may be a synthesized sequence. In further embodiments, the target sequence may be a chromosomal sequence. In certain embodiments, the target sequence may comprise a translocation junction, e.g., a translocation associated with a cancer. In some embodiments, the target sequence may be on a eukaryotic chromosome, such as a human chromosome.

In some embodiments, the target sequence may be located in a genomic locus; for example, the target sequence may be located in a coding sequence of a gene, an intron sequence of a gene, a regulatory sequence, a transcriptional control sequence of a gene, a translational control sequence of a gene, a splicing site, or a non-coding sequence between genes (e.g., intergenic space). In some embodiments, the gene may be a protein coding gene. In other embodiments, the gene may be a non-coding RNA gene. In some embodiments, the target sequence may comprise all or a portion of a disease-associated gene. In some embodiments, the target sequence may be located in a non-genic functional site in the genome, for example a site that controls aspects of chromatin organization, such as a scaffold site or locus control region.

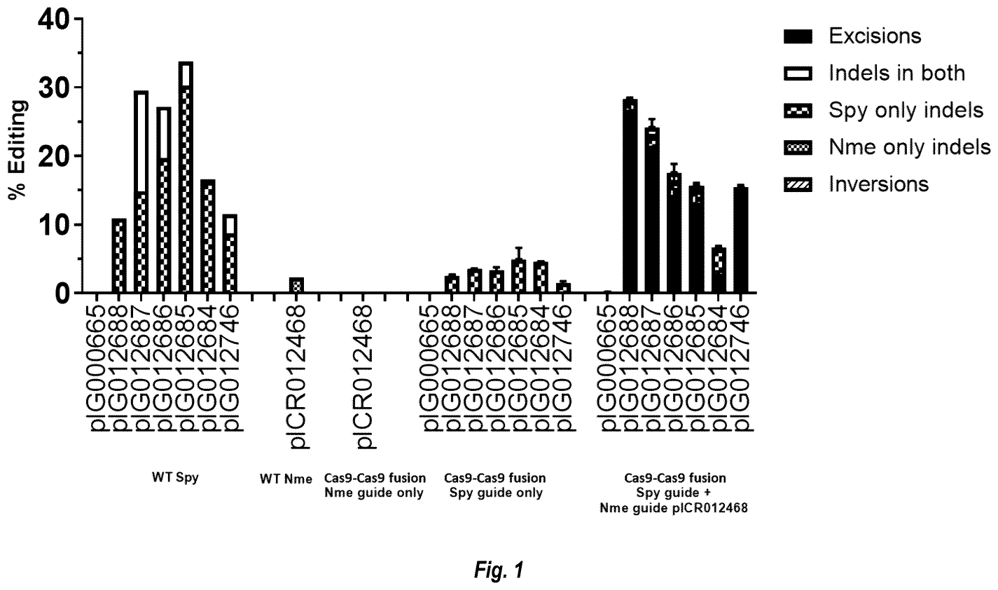

In some embodiments involving a Cas nuclease, such as a Class 2 Cas nuclease, the target sequence may be adjacent to a protospacer adjacent motif (“PAM”). In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Spy Cas9 protein or Spy Cas9 ortholog, including those disclosed in FIG. 1 of Ran et al., Nature, 520: 186-191 (2015), and Figure S5 of Zetsche 2015, the relevant disclosure of each of which is incorporated herein by reference. In some embodiments, the PAM may be 2, 3, 4, 5, 6, 7, 8, 9, or 10 nucleotides in length. Non-limiting exemplary PAM sequences include NGG, NGGNG, NG, NAAAAN, NNAAAAW, NNNNACA, GNNNCNNA, TTN, and NNNNGATT (wherein N is defined as any nucleotide, and W is defined as either A or T). In some embodiments, the PAM sequence may be NGG. In some embodiments, the PAM sequence may be NGGNG. In some embodiments, the PAM sequence may be TTN. In some embodiments, the PAM sequence may be NNAAAAW.

In some embodiments, the PAM may be selected from a consensus or a particular PAM sequence for a specific Nme Cas9 protein or Nme Cas9 ortholog (Edraki et al., 2019). In some embodiments, the Nme Cas9 PAM may comprise 2, 3, 4, 5, 6, 7, 8, 9, or 10 nucleotides in length. Non-limiting exemplary PAM sequences include NCC, N4GAYW, N4GYTT, N4GTCT, NNNNCC(a), NNNNCAAA (wherein N is defined as any nucleotide, W is defined as either A or T, and R is defined as either A or G; and (a) is a preferred, but not required, A after the second C)). In some embodiments, the PAM sequence may be NCC.

In one embodiment, the PAM may be selected from a consensus or a particular PAM sequence for other Class II-C Cas9 orthologs. In some embodiments, the SmuCas9 PAM may comprise one to four required nucleotides selected from the group consisting of N₄CN₃, N₄CT, N₄CCN, N₄CCA, and N₄GNT₃. In one embodiment, the one to four required nucleotides are selected from the group consisting of C, CT, CCN, CCA, CN₃ and GNT₂. In one embodiment, Type II-C Cas9 is bound to a truncated sgRNA.

In some embodiments, the first gRNA that is cognate to the first cleavase or the second gRNA that is cognate to the second cleavase comprises at least one single guide RNA (sgRNA). In some embodiments, the first gRNA that is cognate to the first cleavase or the second gRNA that is cognate to the second cleavase is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification or both.

### B. Guide RNA

In some embodiments, the first guide RNA is a SpyCas9 guide RNA. In the case of a Spy single guide RNA (sgRNA), the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUUGA AAAAGUGGCACCGAGUCGGUGC (SEQ ID NO: 161) in 5′ to 3′ orientation.

In the case of a sgRNA, the above guide sequences may further comprise additional nucleotides to form a sgRNA, e.g., with any one of the following exemplary nucleotide sequence following the 3′ end of the guide sequence: GUUUUAGAGCUAGAAAUAGCAAGUUAAAAUAAGGCUAGUCCGUUAUCAACUUGA AAAAGUGGCACCGAGUCGGUGCUUUU (SEQ ID NO: 160) in 5′ to 3′ orientation; or GUUUUAGAGCUAGAAAUAGCAAGUUAAAAUAAGGCUAGUCCGUUAUCAAAAUGG CACCGAGUCGGUGCU (SEQ ID NO: 165) in 5′ to 3′ orientation.

In the case of a sgRNA, the guide sequences may be integrated into the following modified motif: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 192) where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence. In the context of a modified sequence, unless otherwise indicated, A, C, G, N, and U are an unmodified RNA nucleotide, i.e., a 2′-OH sugar moiety with a phosphodiesterase linkage to the adjacent nucleotide residue, or a 5′-terminal P04.

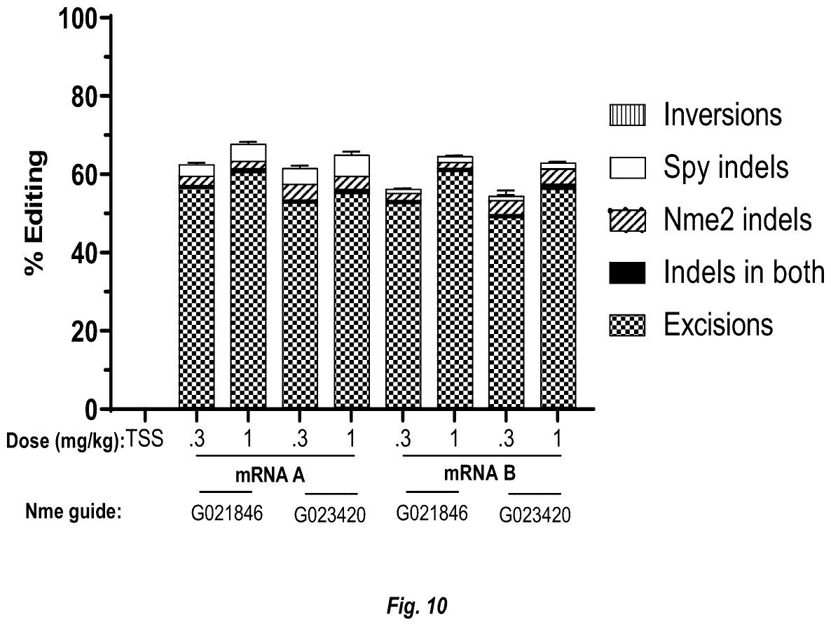

In the case of a sgRNA, the guide sequences may further comprise a SpyCas9 sgRNA sequence. An example of a SpyCas9 sgRNA sequence is shown in Table 39 (SEQ ID NO: 161: GUUUUAGAGCUAGAAAUAGCAAGUUAAAAUAAGGCUAGUCCGUUAUCAACUUGA AAAAGUGGCACCGAGUCGGUGC—“Exemplary SpyCas9 sgRNA-1”), included at the 3′ end of the guide sequence, and provided with the domains as shown in Table 39 below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide; or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 161(“Exemplary SpyCas9 sgRNA-1”) as an example, the Exemplary SpyCas9 sgRNA-1 further includes one or more of: (A) a shortened hairpin 1 region, or a substituted and optionally shortened hairpin 1 region, wherein (1) at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks (a) any one or two of H1-5 through H1-8, (b) one, two, or three of the following pairs of nucleotides: H1-1 and H1-12, H1-2 and H1-11, H1-3 and H1-10, and H1-4 and H1-9, or (c) 1-8 nucleotides of hairpin 1 region; or (2) the shortened hairpin 1 region lacks 4-8 nucleotides, preferably 4-6 nucleotides, and (a) one or more of positions H1-1, H1-2, or H1-3 is deleted or substituted relative to Exemplary SpyCas9 sgRNA-1 (SEQ ID NO: 161), or (b) one or more of positions H1-6 through H1-10 is substituted relative to Exemplary SpyCas9 sgRNA-1(SEQ ID NO: 161); or (3) the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 161); or (B) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 161); or (C) a substitution relative to Exemplary SpyCas9 sgRNA-1 (SEQ ID NO: 161) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or (D) an Exemplary SpyCas9 sgRNA-1 (SEQ ID NO: 161) with an upper stem region, wherein the upper stem modification comprises a modification to any one or more of US1-US12 in the upper stem region, wherein (1) the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof, or (2) the modified nucleotide optionally includes a 2′-OMe modified nucleotide.

In some embodiments, the unmodified sgRNA comprises the following sequence:

(SEQ ID NO: 176)
(N)₂₀GUUUUAGAGCUAGAAAUAGCAAGUUAAAAUAAGGCUAGUCCGUUA
UCACGAAAGGGCACCGAGUCGGUGC;
or
(SEQ ID NO: 177)
(N)₂₀GUUUUAGAGCUAGAAAUAGCAAGUUAAAAUAAGGCUAGUCCGUUA
UCACGAAAGGGCACCGAGUCGGUGCU.

In some embodiments, the sgRNA comprises a modified motif disclosed herein, including any modified motif shown in Tables 2B, 2C, 3B, and 4, where a guide RNA, or “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence.

In the context of a modified sequence, unless otherwise indicated, A, C, G, N, and U are an unmodified RNA nucleotide, i.e., a 2′-OH sugar moiety with a phosphodiester linkage to the adjacent nucleotide residue, or a 5′-terminal PO4.

In some embodiments, the first guide RNA that directs the first cleavase to a first genomic locus is a SpyCas9 guide RNA. In some embodiments, the SpyCas9 guide RNA is a single guide RNA comprising: a conserved portion of an sgRNA comprising an upper stem and hairpin region, wherein every nucleotide in the upper stem region is modified with 2′-O-Me, and every nucleotide in the hairpin region is modified with 2′-O-Me; a 3′ end modification comprising 2′-O-Me modified nucleotides at the last three nucleotides of the 3′ end and phosphorothioate (PS) bonds between the last four nucleotides of the 3′ end; and 5′ end modification comprising 2′-O-Me modified nucleotides at the first three nucleotides of the 5′ end; and phosphorothioate (PS) bonds between the first four nucleotides of the 5′ end.

In some embodiments, the SpyCas9 guide RNA is a short-single guide RNA (short-sgRNA) comprising a conserved portion of an sgRNA comprising a hairpin region, wherein the hairpin region lacks at least 5-10 nucleotides and wherein the short-sgRNA comprises (i) a 5′ end modification or (ii) a 3′ end modification.

In some embodiments, the first guide RNA is a SpyCas9 guide RNA that is a single guide RNA comprising a nucleotide sequence selected from SEQ ID NOs: 159-167, 170-177, and 180-194, or a nucleotide sequence that is at least 85%, at least 90%, or at least 95% identical to SEQ ID NOs: 159-167, 170-177, and 180-194.

In some embodiments, the sgRNA comprises Exemplary SpyCas9 sgRNA-1 or the modified versions thereof provided herein, or a version as provided in Table 2B, where the totality of the N's comprise a guide sequence that directs a nuclease to a target sequence. Each N is independently modified or unmodified. In certain embodiments, in the absence of an indication of a modification, the nucleotide is an unmodified RNA nucleotide residue, i.e., a ribose sugar and a phosphodiester backbone.

TABLE 2A
Exemplary Unmodified Spy Cas9 Scaffold Sequences

| #mer | Unmodified nucleotide sequence | SEQ ID NO |
|---|---|---|
| | GUUUUAGAGCUAUGCUGUUUUG | 159 |
| 100 | GUUUUAGAGCUAGAAAUAGCAAGUUAAAAUAAG | 160 |
| | GCUAGUCCGUUAUCAACUUGAAAAAGUGGCACCG | |
| | AGUCGGUGCUUUU | |
| 96 | GUUUUAGAGCUAGAAAUAGCAAGUUAAAAUAAG | 161 |
| | GCUAGUCCGUUAUCAACUUGAAAAAGUGGCACCG | |
| | AGUCGGUGC | |
| 97 | GUUUUAGAGCUAGAAAUAGCAAGUUAAAAUAAG | 162 |
| | GCUAGUCCGUUAUCAACUUGAAAAAGUGGCACCG | |
| | AGUCGGUGCU | |
| 88 | GUUUUAGAGCUAGAAAUAGCAAGUUAAAAUAAG | 163 |
| | GCUAGUCCGUUAUCAACUUGGCACCGAGUCGGUG | |
| | C | |
| 88 | GUUUUAGAGCUAGAAAUAGCAAGUUAAAAUAAG | 164 |
| | GCUAGUCCGUUAUCAAAAUGGCACCGAGUCGGUG | |
| | C | |
| 89 | GUUUUAGAGCUAGAAAUAGCAAGUUAAAAUAAG | 165 |
| | GCUAGUCCGUUAUCAAAAUGGCACCGAGUCGGUG | |
| | CU | |
| 90 | GUUUUAGAGCUAGAAAUAGCAAGUUAAAAUAAG | 166 |
| | GCUAGUCCGUUAUCACGAAAGGGCACCGAGUCGG | |
| | UGC | |
| 91 | GUUUUAGAGCUAGAAAUAGCAAGUUAAAAUAAG | 167 |
| | GCUAGUCCGUUAUCACGAAAGGGCACCGAGUCGG | |
| | UGCU | |

TABLE 2B
Exemplary Unmodified Spy Cas9 Guide RNA Sequences

| | | SEQ |
|---|---|---|
| #mer | Unmodified nucleotide sequence | ID NO |
| 100 | (N)20GUUUUAGAGCUAGAAAUAGCAAGUUAAAAU | 170 |
| | AAGGCUAGUCCGUUAUCAACUUGAAAAAGUGGC | |
| | ACCGAGUCGGUGCUUUU | |
| 96 | (N)20GUUUUAGAGCUAGAAAUAGCAAGUUAAAAU | 171 |
| | AAGGCUAGUCCGUUAUCAACUUGAAAAAGUGGC | |
| | ACCGAGUCGGUGC | |
| 97 | (N)20GUUUUAGAGCUAGAAAUAGCAAGUUAAAAU | 172 |
| | AAGGCUAGUCCGUUAUCAACUUGAAAAAGUGGC | |
| | ACCGAGUCGGUGCU | |
| 88 | (N)20GUUUUAGAGCUAGAAAUAGCAAGUUAAAAU | 173 |
| | AAGGCUAGUCCGUUAUCAACUUGGCACCGAGUCG | |
| | GUGC | |
| 88 | (N)20GUUUUAGAGCUAGAAAUAGCAAGUUAAAAU | 174 |
| | AAGGCUAGUCCGUUAUCAAAAUGGCACCGAGUCG | |
| | GUGC | |
| 88 | (N)20GGUUUUAGAGCUAGAAAUAGCAAGUUAAAA | 175 |
| | UAAGGCUAGUCCGUUAUCAAAAUGGCACCGAGUC | |
| | GGUGC | |
| 90 | (N)20GUUUUAGAGCUAGAAAUAGCAAGUUAAAAU | 176 |
| | AAGGCUAGUCCGUUAUCACGAAAGGGCACCGAGU | |
| | CGGUGC | |
| 91 | (N)20GUUUUAGAGCUAGAAAUAGCAAGUUAAAAU | 177 |
| | AAGGCUAGUCCGUUAUCACGAAAGGGCACCGAGU | |
| | CGGUGCU | |

wherein the Ns collectively are a guide sequence provided herein. Within the table, in the context of an unmodified sequence, A, C, G, U, and N are, independently, any natural or non-natural adenine, cytosine, guanine, uracil, and any nucleotide (e.g., A, C, G, or U), respectively.

TABLE 2C
Exemplary Modified Spy Guide Scaffold Sequences

| | | SEQ |
|---|---|---|
| | | ID |
| #mer | Modified sequence | NO |
| 100 | GUUUUAGAmGmCmUmAmGmAmAmAmUmAmGmCAAGUUAA | 180 |
| | AAUAAGGCUAGUCCGUUAUCAmAmCmUmUmGmAmAmAmA | |
| | mAmGmUmGmGmCmAmCmCmGmAmGmUmCmGmGmUmGmCm | |
| | U*mU*mU*mU | |
| 96 | GUUUUAGAmGmCmUmAmGmAmAmAmUmAmGmCAAGUUAA | 181 |
| | AAUAAGGCUAGUCCGUUAUCAmAmCmUmUmGmAmAmAmA | |
| | mAmGmUmGmGmCmAmCmCmGmAmGmUmCmGmGmUmGmC | |
| 97 | GUUUUAGAmGmCmUmAmGmAmAmAmUmAmGmCAAGUUAA | 182 |
| | AAUAAGGCUAGUCCGUUAUCAmAmCmUmUmGmAmAmAmA | |
| | mAmGmUmGmGmCmAmCmCmGmAmGmUmCmGmGmUmGmCm | |
| | U | |
| 88 | GUUUUAGAmGmCmUmAmGmAmAmAmUmAmGmCAAGUUAA | 183 |
| | AAUAAGGCUAGUCCGUUAUCAACUUGGCACCGAGUCGG* | |
| | mU*mG*mC | |
| 88 | GUUUUAGAmGmCmUmAmGmAmAmAmUmAmGmCAAGUUAA | 184 |
| | AAUAAGGCUAGUCCGUUAUCAAAAUGGCACCGAGUCGG* | |
| | mU*mG*mC | |
| 88 | GUUUUAGAmGmCmUmAmGmAmAmAmUmAmGmCAAGUUAA | 185 |
| | AAUAAGGCUAGUCCGUUAUCAmAmAmAmUmGmGmCmAmC | |
| | mCmGmAmGmUmCmGmG*mU*mG*mC | |
| 90 | GUUUUAGAmGmCmUmAmGmAmAmAmUmAmGmCAAGUUAA | 186 |
| | AAUAAGGCUAGUCCGUUAUCACGAAAGGGCACCGAGUCG | |
| | G*mU*mG*mC | |
| 90 | GUUUUAGAmGmCmUmAmGmAmAmAmUmAmGmCAAGUUAA | 187 |
| | AAUAAGGCUAGUCCGUUAUCAmCmGmAmAmAmGmGmGmC | |
| | mAmCmCmGmAmGmUmCmGmG*mU*mG*mC | |
| 91 | GUUUUAGAmGmCmUmAmGmAmAmAmUmAmGmCAAGUUAA | 188 |
| | AAUAAGGCUAGUCCGUUAUCACGAAAGGGCACCGAGUCG | |
| | GU*mG*mC*mU | |
| 91 | GUUUUAGAmGmCmUmAmGmAmAmAmUmAmGmCAAGUUAA | 189 |
| | AAUAAGGCUAGUCCGUUAUCACGAAAGGGCACCGAGUCG | |
| | GmUmGmC*mU | |
| 91 | GUUUfUAGmAmGmCmUmAmGmAmAmAmUmAmGmCmAmAG | 190 |
| | UfUmAfAmAfAmUAmAmGmGmCmUmAGUmCmCGUfUAmU | |
| | mCAmCmGmAmAmAmGmGmGmCmAmCmCmGmAmGmUmCmG | |
| | mGmU*mG*mC*mU | |
| 91 | GUUUUAGAmGmCmUmAmGmAmAmAmUmAmGmCAAGUUAA | 191 |
| | AAUAAGGCUAGUCCGUUAUCACGAAAGGGCACCGAGUCG | |
| | GmU*mG*mC*mU | |

wherein “m” indicates a 2′-O-Me modification, “f” indicates a 2′-fluoro modification, a “*” indicates a phosphorothioate linkage between nucleotides, and no modification in the context of a modified sequence indicates an RNA (2′-OH) and phosphodiesterase linkage to the 3′ nucleotide when one is present.

In certain embodiments, the guide sequence is a chemically modified sequence. In certain embodiments, the chemically modified guide sequence is (mN*)3(N)13-17. In certain embodiments, the guide sequence is (mN*)3(N)17, i.e., mN*mN*mN*NNNNNNNNNNNNNNNNN. In certain embodiments, each N of the (N)13-17 or the (N)17 is unmodified. In certain embodiments, each N in the (N)13-17 or the (N)17 is independently modified, e.g., independently modified with a 2′-O-methyl modification.

In some embodiments, the sgRNA disclosed herein may be modified as shown herein or in the sequence mN*mN*mN*NNNNNNNNNNNNNNNNN GUUUUAGAmGmCmUmAmGmAmAmAmUmAmGmCAAGUUAAAAUAAGGCUAGUCC GUUAUCACGAAAGGGCACCGAGUCGG*mU*mG*mC (SEQ ID NO: 193).
In some embodiments, the sgRNA disclosed herein may be modified as shown herein or in the sequence mN*mN*mN*NNNNNNNNNNNNNNNNN GUUUUAGAmGmCmUmAmGmAmAmAmUmAmGmCAAGUUAAAAUAAGGCUAGUCC GUUAUCACGAAAGGGCACCGAGUCGGmU*mG*mC*mU (SEQ ID NO: 194).

In some embodiments, the guide RNA is a Campylobacter jejuni Cas9 (“CjeCas9”) guide RNA. In some embodiments, the guide RNA is a modified CjeCas9 guide RNA.

In some embodiments, the guide RNA is a Simonsiella muelleri Cas9 (“SmuCas9”) guide RNA. In some embodiments, the guide RNA is a modified SmuCas9 guide RNA.

In some embodiments, the second guide RNA disclosed herein is a NmeCas9 guide RNA. In some embodiments, the second guide RNA is a NmeCas9 guide RNA that is a single guide RNA comprising a nucleotide sequence selected from SEQ ID NOs: 280-297, or a nucleotide sequence that is at least 85%, at least 90%, or at least 95% identical to SEQ ID NOs: 280-297. In some embodiments, the second guide comprises one or more internal polyethylene glycol (PEG) linker. In some embodiments, the second guide RNA comprising internal linkers comprise a sequence selected from SEQ ID NOs: 161-169.

In certain embodiments, using SEQ ID NO: 279 (“Exemplary NmeCas9 sgRNA-1”) as an example, the Exemplary NmeCas9 sgRNA-1 includes: (A) A guide RNA (gRNA) comprising a guide region and a conserved region, the conserved region comprising one or more of: (a) a shortened repeat/anti-repeat region, wherein the shortened repeat/anti-repeat region lacks 2-24 nucleotides, wherein (i) one or more of nucleotides 37-48 and 53-64 is deleted and optionally one or more of nucleotides 37-64 is substituted relative to SEQ ID NO: 279; and (ii) nucleotide 36 is linked to nucleotide 65 by at least 2 nucleotides; or (b) a shortened hairpin 1 region, wherein the shortened hairpin 1 lacks 2-10, optionally 2-8 nucleotides, wherein (i) one or more of nucleotides 82-86 and 91-95 is deleted and optionally one or more of positions 82-96 is substituted relative to SEQ ID NO: 279; and (ii) nucleotide 81 is linked to nucleotide 96 by at least 4 nucleotides; or (c) a shortened hairpin 2 region, wherein the shortened hairpin 2 lacks 2-18, optionally 2-16 nucleotides, wherein (i) one or more of nucleotides 113-121 and 126-134 is deleted and optionally one or more of nucleotides 113-134 is substituted relative to SEQ ID NO: 279; and (ii) nucleotide 112 is linked to nucleotide 135 by at least 4 nucleotides; wherein one or both nucleotides 144-145 are optionally deleted relative to SEQ ID NO: 279; wherein optionally at least 10 nucleotides are modified nucleotides.

Exemplary unmodified conserved portion nucleotide sequences are provided in Table 3A.

In the case of a sgRNA, the guide sequences may be integrated into one of the following exemplary modified conserved portion motifs as shown in Table 3B.

In certain embodiments, the guide sequence is 20-25 nucleotides in length ((N)20-25), wherein each nucleotide may be independently modified. In certain embodiments, each of nucleotides 1-3 of the 5′ end of the guide is independently modified. In certain embodiments, each of nucleotides 1-3 of the 5′ end of the guide is independently modified with a 2′-OMe modification. In certain embodiments, each of nucleotides 1-3 of the 5′ end of the guide is independently modified with a phosphorothioate linkage to the adjacent nucleotide residue. In certain embodiments, each of nucleotides 1-3 of the 5′ end of the guide is independently modified with a 2′-OMe modification and a phosphorothioate linkage to the adjacent nucleotide residue.

In the case of a sgRNA, modified guide sequences may be integrated into one of the following exemplary modified conserved portion motifs as shown in Table 3B.

In some embodiments, the guide RNA comprises a sgRNA comprising a guide region and a conserved portion of an sgRNA, for example, the conserved portion of sgRNA shown as Exemplary NmeCas9 sgRNA-1 or the conserved portions of the gRNAs shown in Table 3A-31B and throughout the specification.

In some embodiments, the sgRNA comprises Exemplary NmeCas9 sgRNA-1 or the modified versions thereof provided herein, or a version as provided in Table 3B. Each N is independently modified or unmodified. In certain embodiments, in the absence of an indication of a modification, the nucleotide is an unmodified RNA nucleotide residue, i.e., a ribose sugar and a phosphodiester backbone.

TABLE 3A
Unmodified Nme Conserved Region
Nucleotide Sequences

| | Unmodified nucleotide | SEQ |
|---|---|---|
| #mer | sequence of conserved portion | ID NO |
| 145 | GUUGUAGCUCCCUUUCUCAUUUCGGAAACGAAAUGAGAACC | 280 |
| | GUUGCUACAAUAAGGCCGUCUGAAAAGAUGUGCCGCAACGC | |
| | UCUGCCCCUUAAAGCUUCUGCUUUAAGGGGCAUCGUUUA | |
| 101 | GUUGUAGCUCCCUGAAACCGUUGCUACAAUAAGGCCGUCGA | 281 |
| | AAGAUGUGCCGCAACGCUCUGCCUUCUGGCAUCGUU | |
| 105 | GUUGUAGCUCCCUUCGAAAGACCGUUGCUACAAUAAGGCCG | 282 |
| | UCGAAAGAUGUGCCGCAACGCUCUGCCUUCUGGCAUCGUU | |
| 107 | GUUGUAGCUCCCUGGAAACCCGUUGCUACAAUAAGGCCGUC | 283 |
| | GAAAGAUGUGCCGCAACGCUCUGCCUUCUGGCAUCGUUUAU | |
| | U | |

TABLE 3B
Exemplary Modified Nme Guide RNA Motifs

| | Modified nucleotide sequence | SEQ |
|---|---|---|
| #mer | of conserved portion | ID NO |
| 101 | mGUUGmUmAmGmCUCCCmUmGmAmAmAmCmCGUUmGmCUA | 284 |
| | mCAAU*AAGmGmCCmGmUmCmGmAmAmAmGmAmUGUGCmC | |
| | GCmAmAmCmGCUCUmGmCCmUmUmCmUGmGCmAmUC*mG* | |
| | mU*mU | |
| 101 | mGUUGmUmAmGmCUCCCmUmGmAmAmAmCmCGUUmGmCUA | 285 |
| | mCAAU*AAGmGmCCmGmUmCmGmAmAmAmGmAmUGUGCmC | |
| | GmCAAmCGCUCUmGmCCmUmUmCmUGGCAUCG*mU*mU | |
| 101 | mGUUGmUmAmGmCUCCCmUmGmAmAmAmCmCGUUmGmCUA | 286 |
| | mCAAUAAGmGmCCmGmUmCmGmAmAmAmGmAmUGUGCmCG | |
| | mCAAmCGCUCUmGmCCmUmUmCmUGGCAUCG*mU*mU | |
| 101 | mGUUGmUmAmGmCUCCCmUmGmAmAmAmCmCGUUmGmCUA | 287 |
| | mCAAU*AAGmGmCCmGmUmCmGmAmAmAmGmAmUGUGCmC | |
| | GmCAAmCGmCmUmCmUmGmCCmUmUmCmUGGCAUCG*mU*m | |
| | U | |
| 101 | mGUUGmUmAmGmCUCCCmUmGmAmAmAmCmCGUUmGmCUA | 288 |
| | mCAAUAAGmGmCCmGmUmCmGmAmAmAmGmAmUGUGCmCG | |
| | mCAAmCGmCmUmCmUmGmCCmUmUmCmUGGCAUCG*mU*mU | |
| 105 | mGUUGmUmAmGmCUCCCmUmUmCmGmAmAmAmGmAmCmCG | 289 |
| | UUmGmCUAmCAAU*AAGmGmCCmGmUmCmGmAmAmAmGmA | |
| | mUGUGCmCGmCAAmCGCUCUmGmCCmUmUmCmUGGCAUCG* | |
| | mU*mU | |
| 105 | mGUUGmUmAmGmCUCCCmUmUmCmGmAmAmAmGmAmCmCG | 290 |
| | UUmGmCUAmCAAUAAGmGmCCmGmUmCmGmAmAmAmGmAm | |
| | UGUGCmCGmCAAmCGCUCUmGmCCmUmUmCmUGGCAUCG*m | |
| | U*mU | |
| 105 | mGUUGmUmAmGmCUCCCmUmUmCmGmAmAmAmGmAmCmCG | 291 |
| | UUmGmCUAmCAAU*AAGmGmCCmGmUmCmGmAmAmAmGmA | |
| | mUGUGCmCGmCAAmCGmCmUmCmUmGmCCmUmUmCmUGGC | |
| | AUCG*mU*mU | |
| 105 | mGUUGmUmAmGmCUCCCmUmUmCmGmAmAmAmGmAmCmCG | 292 |
| | UUmGmCUAmCAAUAAGmGmCCmGmUmCmGmAmAmAmGmAm | |
| | UGUGCmCGmCAAmCGmCmUmCmUmGmCCmUmUmCmUGGCA | |
| | UCG*mU*mU | |
| | mN*mNNNNNNNNmNNNmNNNNNNNNNNNNmGUUGmUmAmG | 293 |
| | mCUCCCmUmGmAmAmAmCmCGUUmGmCUAmCAAU*AAGmG | |
| | mCCmGmUmCmGmAmAmAmGmAmUGUGCmCGCmAmAmCmGC | |
| | UCUmGmCCmUmUmCmUGmGCmAmUC*mG*mU*mU | |
| | (N)20-25 | 294 |
| | GUUGmUmAmGmCUCCCmUmGmAmAmAmCmCGUUmGmCUAm | |
| | CAAU*AAGmGmCCmGmUmCmGmAmAmAmGmAmUGUGCmCG | |
| | CmAmAmCmGCUCUmGm | |
| | CCmUmUmCmUGmGCmAmUC*mG*mU*mU | |
| | mN*mN*mN*mNmNNNmNmNNmNNmNNNNNmNNNNmNNNmG | 295 |
| | UUGmUmAmGmCUCCCmUmGmAmAmAmCmCGUUmGmCUAmC | |
| | AAU*AAGmGmCCmGmUmCmGmAmAmAmGmAmUGUGCmCGm | |
| | CAAmCGCUCUmGmCCmUmUmCmUGGCAUCG*mU*mU | |
| | mN*mN*mN*N(21)GUUGUAGCUCCCU*mG*mA*mA*mA*CCGU | 296 |
| | UGCUACAAUAAGGCCGUmC*mU*mG*mA*mA*mA*mA*mGAU | |
| | GUGCCGCAACGCUCUGCCG*mC*mU*mU*mC*mU*mG*CGGCA | |
| | UCGUUU*mA*mU*mC | |
| | mN*mN*mN*N(21)GUUGUAGCUCCCU*mG*mA*mA*mA*CCGU | 297 |
| | UGCUACAAUAAGGCCGUmC*mG*mA*mA*mA*mGAUGUGCCG | |
| | CAACGCUCUGCCmU*mU*mC*mUGGCAUCGUUU*mA*mU*mC | |

wherein “m” indicates a 2'-O-Me modification, and a “*” indicates a phosphorothioate linkage between nucleotides, and no modification in the context of a modified sequence indicates an RNA (2'-OH) and a phosphorothioate linkage.

In certain embodiments, the guide sequence is a chemically modified sequence. In certain embodiments, the chemically modified guide sequence is (mN*)3(N)17-22. In certain embodiments, the guide sequence is (mN*)3(N)21, i.e., mN*mN*mN*NNNNNNNNNNNNNNNNNNNNN. In certain embodiments, each N of the (N)17-22 or the (N)21 is unmodified. In certain embodiments, the each N in the (N)18-21 or the (N)21 is independently modified, e.g. independently modified with a 2′-O-methyl modification.

The shortened NmeCas9 gRNA may comprise internal linkers disclosed herein. “Internal linker” as used herein describes a non-nucleotide segment joining two nucleotides within a guide RNA. If the gRNA contains a guide region, the internal linker is located outside of the spacer region (e.g., in the scaffold or conserved region of the gRNA). For Type V guides, it is understood that the last hairpin is the only hairpin in the structure, i.e., the repeat-anti-repeat region. In some embodiments, the internal linker comprises a PEG-linker disclosed herein. Exemplary locations of the linkers are as shown in the following: (N)₂₀₋₂₅ GUUGUAGCUCCCUUC(L1)GACCGUUGCUACAAUAAGGCCGUC(L1)GAUGU GCCGCAACGCUCUGCC(L1)GGCAUCGUU (SEQ ID NO: 272). As used herein, (L1) refers to an internal linker having a bridging length of about 15-21 atoms. In some embodiments, the internal linker comprises a polyethylene glycol (PEG) linker. The guide RNAs comprising an internal linker disclosed herein comprise one of the structures/modification patterns disclosed in WO2022/261292, the contents of which are hereby incorporated by reference in its entirety. Further exemplary NmeGuide RNA comprising linkers are provided in Table 4.

In some embodiments, the shortened NmeCas9 guide RNA comprising internal linkers may be chemically modified as shown in Table 4. In certain embodiments, the guide sequence is a chemically modified sequence as shown in Table 4.

TABLE 4
Exemplary Modified Nme Guide RNA Motifs

| SEQ | | |
|---|---|---|
| ID | | |
| NO | Description | Sequence |
| 272 | Shortened/unmodified | (N)₂₀₋₂₅ |
| | NmeCas9 guide RNA | GUUGUAGCUCCCUUC(L1)GACCGUUGCUAC |
| | comprising linkers | AAUAAGGCCGUC(L1)GAUGUGCCGCAACGC |
| | | UCUGCC(L1)GGCAUCGUU |
| 273 | Shortened/modified NmeCas9 | GUUGmUmAmGmCUCCCmUmUmC(L1)mGmA |
| | guide RNA comprising linkers | mCmCGUUmGmCUAmCAAU*AAGmGmCCmG |
| | (conserved portion only) | mUmC(L1)mGmAmUGUGCmCGmCAAmCGCU |
| | | CUmGmCC(L1)GGCAUCG*mU*mU |
| 274 | Shortened/modified NmeCas9 | GUUGmUmAmGmCUCCCUmC(L1)mGCCGUU |
| | guide RNA comprising linkers | mGmCUAmCAAU*A*AGmGmCCmGmUmC(L1) |
| | (conserved portion only) | mGmAmUGUGCmCGCAACGCUCUmGmCC(L1) |
| | | GGCAUCGUUU*AmU*mU |
| 275 | Shortened/modified NmeCas9 | GUUGmUmAmGmCUCCCmUmUmC(L1)mGmA |
| | guide RNA comprising linkers | mCmCGUUmGmCUAmCAAU*AAGmGmCCmG |
| | (conserved portion only) | mUmC(L1)mGmAmUGUGCmCGmCAAmCGCU |
| | | CUmGmCC(L1)GGCAUCGmU*mU |
| 276 | Shortened/modified NmeCas9 | mN*mN*mN*mNmNNNmNmNNmNNmNNNNN |
| | guide RNA comprising linkers | mNNNNmNNNmGUUGmUmAmGmCUCCCmU |
| | | mUmC(L1)mGmAmCmCGUUmGmCUAmCAAU |
| | | *AAGmGmCCmGmUmC(L1)mGmAmUGUGCm |
| | | CGmCAAmCGCUCUmGmCC(L1)GGCAUCG*m |
| | | U*mU |
| 277 | Shortened/modified NmeCas9 | mN*mNN*NNNNNNmNNNmNNNNNNNNNNN |
| | guide RNA comprising linkers | mGUUGmUmAmGmCUCCCUmC(L1)mGCCGU |
| | | UmGmCUAmCAAU*A*AGmGmCCmGmUmC |
| | | (L1)mGmAmUGUGCmCGCAACGCUCUmGmC |
| | | C(L1)GGCAUCGUUU*AmU*mU |
| 278 | Shortened/modified NmeCas9 | mN*mN*mN*mNmNNNmNmNNmNNmNNNNN |
| | guide RNA comprising linkers | mNNNNmNNNmGUUGmUmAmGmCUCCCmU |
| | | mUmC(L1)mGmAmCmCGUUmGmCUAmCAAU |
| | | *AAGmGmCCmGmUmC(L1)mGmAmUGUGCm |
| | | CGmCAAmCGCUCUmGmCC(L1)GGCAUCGmU |
| | | *mU |

In certain embodiments, Exemplary SpyCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a sequence substituted nucleotide, wherein the pyrimidine is substituted for a purine. In certain embodiments, when the pyrimidine forms a Watson-Crick base pair in the single guide, the Watson-Crick based nucleotide of the sequence substituted pyrimidine nucleotide is substituted to maintain Watson-Crick base pairing.

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of the 3′ end or 5′ end of the oligonucleotide to provide exonuclease stability, e.g., with 2′ O-me, 2′ halide, or 2′ deoxy substituted ribose; or inverted abasic terminal nucleotide, or replacement of phosphodiester with phosphothioate.

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In certain embodiments, all, or substantially all, of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, preferably at least 20%, at least 25%, at least 30%, at least 35%, at least 40%, at least 45%, or at least 50%) of the positions in a modified gRNA are modified nucleosides or nucleotides. In some embodiments, at least 5% of the positions in the modified guide RNA are modified nucleotides or nucleosides. In some embodiments, at least 10% of the positions in the modified guide RNA are modified nucleotides or nucleosides. In some embodiments at least 15% of the positions in the modified gRNA are modified nucleotides or nucleosides. In some embodiments preferably at least 20% of the positions in the modified gRNA are modified nucleotides or nucleosides. In some embodiments, no more than 65% of the positions in the modified gRNA are modified nucleotides. In some embodiments, no more than 55% of the positions in the modified gRNA are modified nucleotides. In some embodiments, no more than 50% of the positions in the modified gRNA are modified nucleotides. In some embodiments, 10-70% of the positions in the modified gRNA are modified nucleotides. In some embodiments, 20-70% of the positions in the modified gRNA are modified nucleotides. In some embodiments, 20-50% of the positions in the modified gRNA are modified nucleotides and the nuclease is a SpyCas9 nuclease. In some embodiments, 30-70% of the positions in the modified gRNA are modified nucleotides and the nuclease is an NmeCas9 nuclease.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, borano phosphate esters, methyl phosphonates, phosphoroamidates, phosphodithioate, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications, e.g., an amide linkage. In some embodiments, the charged phosphate group can be replaced by a neutral moiety. Examples of moieties which can replace the phosphate group can include, without limitation, e.g., methyl phosphonate, carboxymethyl, carbamate, amide, thioether. Further examples of moieties which can replace the phosphate group can include, without limitation, e.g., ethylene oxide linker, sulfonate, sulfonamide, thioformacetal, formacetal, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative). 2′ modifications can include hydrogen (i.e. deoxyribose sugars); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g., L-nucleosides. As used herein, a single abasic sugar is not understood to result in a discontinuity of a duplex.

In certain embodiments, 2′ modifications, include, for example, modifications include 2′-OMe, 2′-F, 2′-H, optionally 2′-O-Me.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the sgRNA may be chemically modified throughout. Certain embodiments comprise a 5′ end modification. Certain embodiments comprise a 3′ end modification. Certain embodiments comprise a 5′ end modification and a 3′ end modification.

In some embodiments, the guide RNAs disclosed herein comprise one of the modification patterns disclosed in WO2018/107028, the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety. In some embodiments, the guide RNAs disclosed herein comprise one of the structures/modification patterns disclosed in WO2019/237069, the contents of which are hereby incorporated by reference in their entirety. In some embodiments, the guide RNAs disclosed herein comprise one of the structures/modification patterns disclosed in WO2021/119275, the contents of which are hereby incorporated by reference in their entirety. In some embodiments, the guide RNAs disclosed herein comprise one of the structures/modification patterns disclosed in PCT/US2022/079121, the contents of which are hereby incorporated by reference in their entirety. In some embodiments, the guide RNAs disclosed herein comprise one of the structures/modification patterns disclosed in WO2022/261292, the contents of which are hereby incorporated by reference in their entirety.

### VI. Delivery

The following section provides additional features of lipid-based delivery compositions, including lipid nanoparticles (LNPs) and lipoplexes, for the nucleic acid described herein or nucleic acids encoding polypeptide disclosed herein. In some embodiments, the nucleic acid or nucleic acids encoding the same is delivered to the cell via at least one lipid nanoparticle (LNP).

In some embodiments, LNP refers to lipid nanoparticles with a diameter of <100 nM, or a population of LNP with an average diameter of <100 nM. In certain embodiments, an LNP has a diameter of about 1-250 nm, about 10-200 nm, about 20-150 nm, about 50-150 nm, about 50-100 nm, about 50-120 nm, about 60-100 nm, about 75-150 nm, about 75-120 nm, or about 75-100 nm, or a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm. In preferred embodiments, an LNP composition has a diameter of 75-150 nm.

LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e., more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo ACT therapy. In some embodiments, the aqueous solution comprises an RNA, such as an mRNA or a gRNA. In some embodiments, the aqueous solution comprises an mRNA encoding an RNA-guided DNA binding agent, such as Cas9.

In some embodiments, the lipid nucleic acid assembly formulations include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

### A. Amine Lipids

In some embodiments, LNPs comprise an ionizable lipid such as Lipid A, or Lipid D or their equivalents, including acetal analogs of Lipid A or Lipid D.

In some embodiments, the ionizable lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

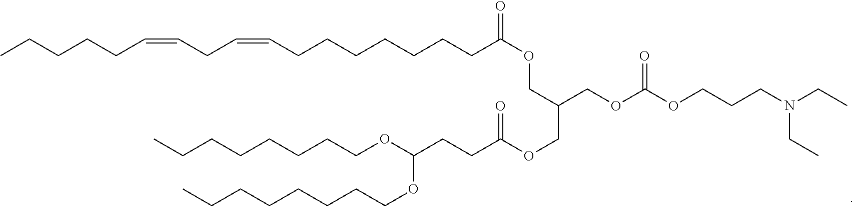 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is Lipid A, or an amine lipid provided in WO2020/219876, which is hereby incorporated by reference.

In some embodiments, an ionizable lipid is an analog of Lipid A. In some embodiments, a Lipid A analog is an acetal analog of Lipid A. In particular LNPs, the acetal analog is a C₄-C₁₂ acetal analog. In some embodiments, the acetal analog is a C₅-C₁₂ acetal analog. In additional embodiments, the acetal analog is a C₅-C₁₀ acetal analog. In further embodiments, the acetal analog is chosen from a C₄, C₅, C₆, C₇, C₉, C₁₀, C₁₁, and C₁₂ acetal analog.

In some embodiments, the ionizable lipid is a compound having a structure of Formula IA

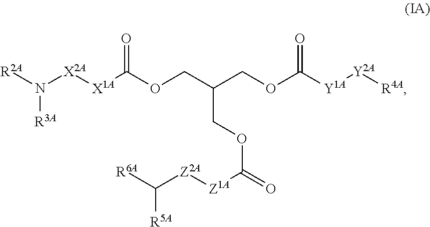

wherein
X^{1A} is O, NH, or a direct bond;
X^{2A} is C₂₋₃ alkylene;
R^{3A} is C₁₋₃ alkyl;
R^{2A} is C₁₋₃ alkyl, or
R^{2A} taken together with the nitrogen atom to which it is attached and 2-3 carbon atoms of X^{2A} form a 5- or 6-membered ring, or
R^{2A} taken together with R3A and the nitrogen atom to which they are attached form a 5-membered ring;
Y^{1A} is C₆₋₁₀ alkylene;
Y^{2A} is selected from

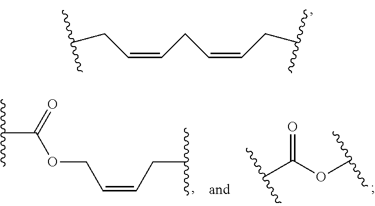 [SMILES: *C/C=C\C/C=C\C* ; *C/C=C\COC(*)=O ; *OC(*)=O]

R^{4A} is C₄₋₁₁ alkyl;
Z^{1A} is C₂₋₅ alkylene;
Z^{2A} is

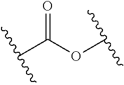 [SMILES: *OC(*)=O]

or absent;
R^{5A} is C₆₋₈ alkyl or C₆₋₈ alkoxy; and
R^{6A} is C₆₋₈ alkyl or C₆₋₈ alkoxy
or a salt thereof.

In some embodiments, the amine lipid is a compound of Formula (IIA)

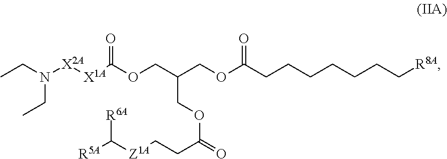

wherein
X^{1A} is O, NH, or a direct bond;
X^{2A} is C₂₋₃ alkylene;
Z^{1A} is C₃ alkylene and R^{5A} and R^{6A} are each C₆ alkyl, or Z^{1A} is a direct bond and R^{5A} and R^{6A} are each C₈ alkoxy; and
R^{8A} is

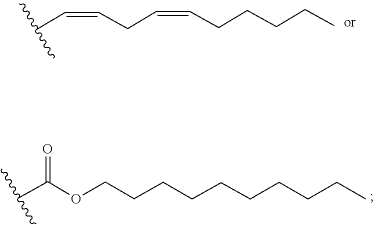 [SMILES: */C=C\C/C=C\CCCCC ; *C(=O)OCCCCCCCCCC]

or a salt thereof.

In certain embodiments, X^{1A} is O. In other embodiments, X^{1A} is NH. In still other embodiments, X^{1A} is a direct bond.

In certain embodiments, X^{1A} is C₃ alkylene. In particular embodiments, X^{2A} is C₂ alkylene.

In certain embodiments, Z1A is a direct bond and R^{5A} and R^{6A} are each C₈ alkoxy. In other embodiments, Z^{1A} is C₃ alkylene and R^{5A} and R^{6A} are each C₆ alkyl.

In certain embodiments, R^{8A} is

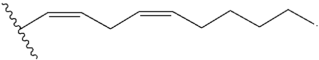

In other embodiments, R^{8A} is

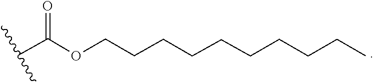 [SMILES: *C(=O)OCCCCCCCCCC]

In certain embodiments, the amine lipid is a salt.

Representative compounds of Formula (IA) include:

| Compound | |
|---|---|
| Number | Compound |
| 1A | 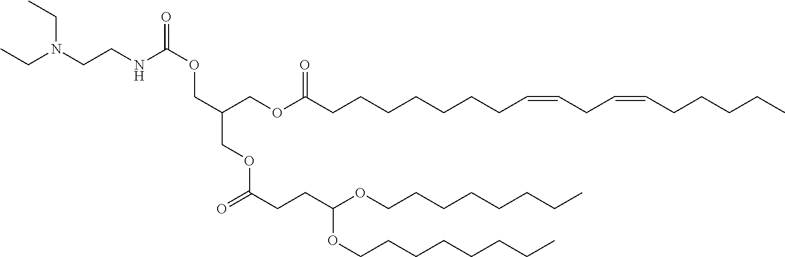 [SMILES: CCCCC/C=C\C/C=C\CCCCCCCC(=O)OCC(COC(=O)CCC(OCCCCCCCC)OCCCCCCCC)COC(=O)NCCN(CC)CC] |
| 2A | 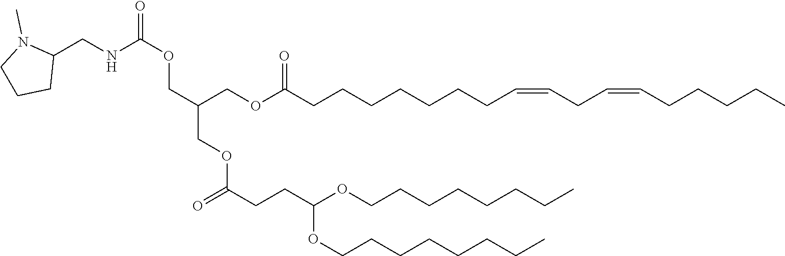 [SMILES: CCCCC/C=C\C/C=C\CCCCCCCC(=O)OCC(COC(=O)CCC(OCCCCCCCC)OCCCCCCCC)COC(=O)NCC1CCCN1C] |
| 3A | 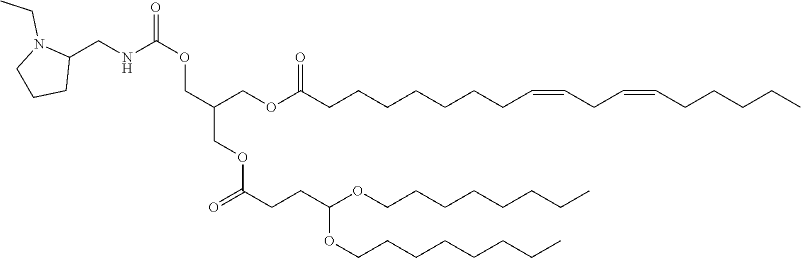 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 4A | 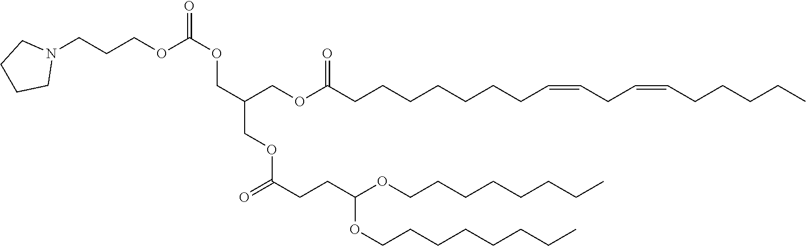 [SMILES: CCCCC/C=C\C/C=C\CCCCCCCC(=O)OCC(COC(=O)CCC(OCCCCCCCC)OCCCCCCCC)COC(=O)OCCCN1CCCC1] |
| 5A | 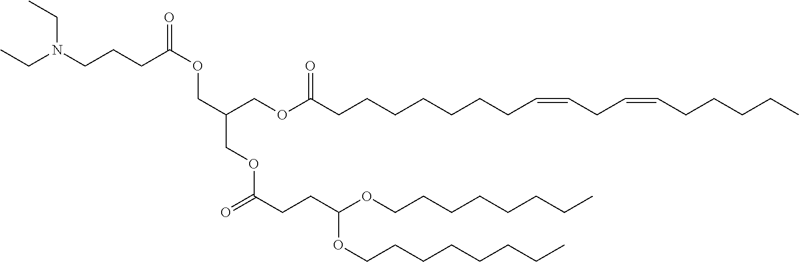 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 6A | 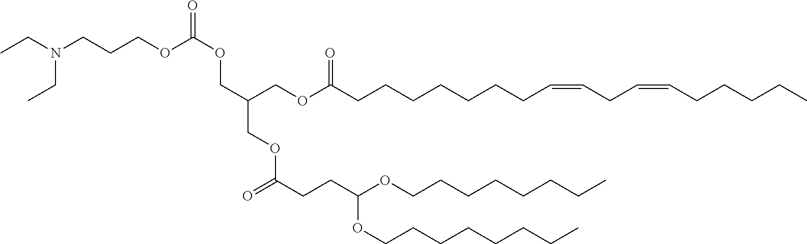 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 7A | 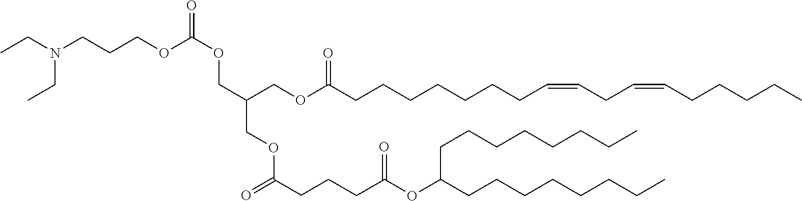 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC] |
| 8A | 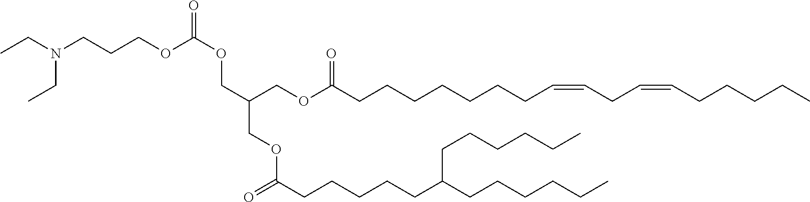 [SMILES: CCCCC/C=C\C/C=C\CCCCCCCC(=O)OCC(COC(=O)CCCCCC(CCCCCC)CCCCCC)COC(=O)OCCCN(CC)CC] |
| 9A | 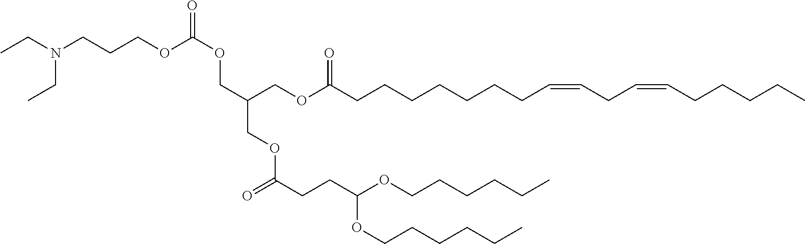 [SMILES: CCCCC/C=C\C/C=C\CCCCCCCC(=O)OCC(COC(=O)CCC(OCCCCCC)OCCCCCC)COC(=O)OCCCN(CC)CC] |
| 10A | 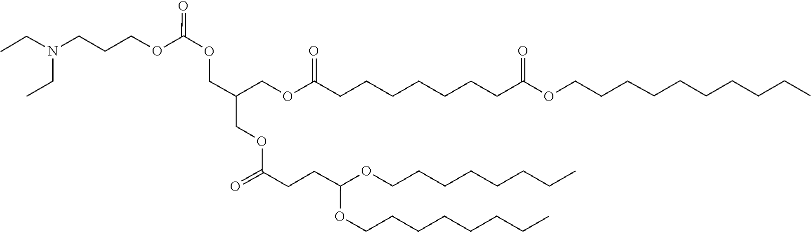 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 11A | 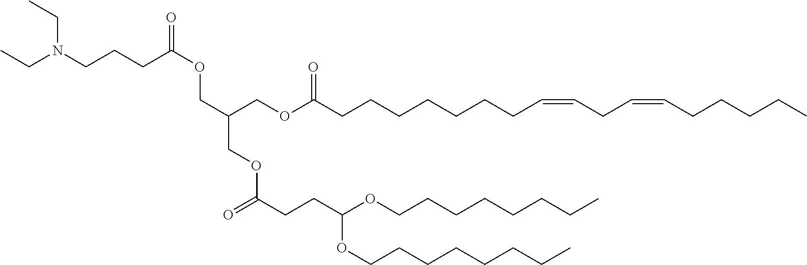 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 12A | 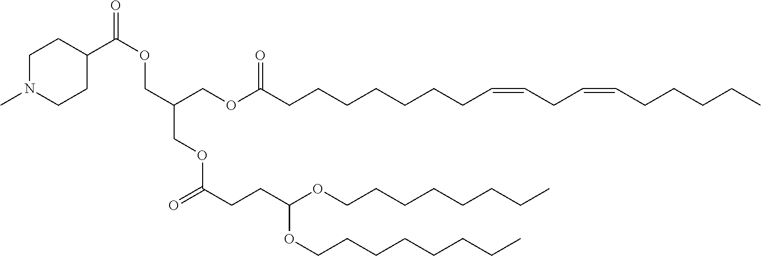 [SMILES: CCCCC/C=C\C/C=C\CCCCCCCC(=O)OCC(COC(=O)CCC(OCCCCCCCC)OCCCCCCCC)COC(=O)C1CCN(C)CC1] |
| 13A | 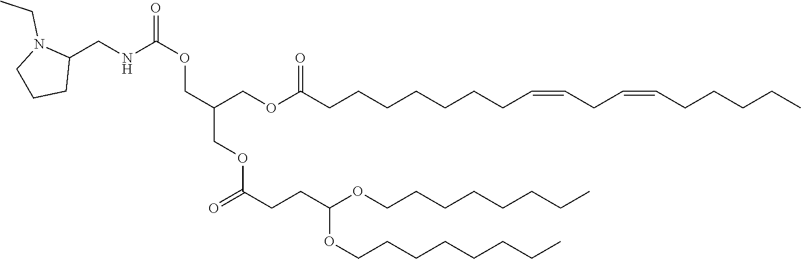 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 14A | 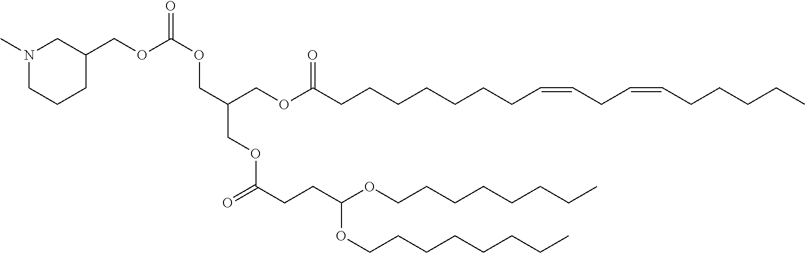 [SMILES: CCCCC/C=C\C/C=C\CCCCCCCC(=O)OCC(COC(=O)CCC(OCCCCCCCC)OCCCCCCCC)COC(=O)OCC1CCCN(C)C1] |
| 15A | 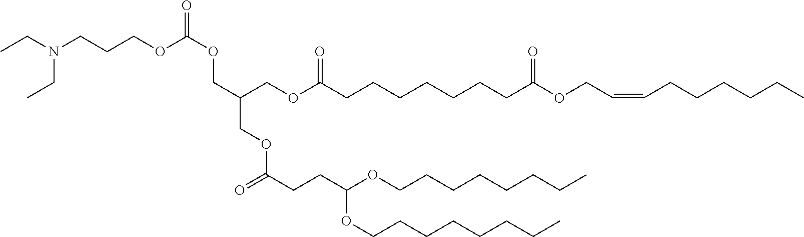 [SMILES: CCCCCC/C=C\COC(=O)CCCCCCCC(=O)OCC(COC(=O)CCC(OCCCCCCCC)OCCCCCCCC)COC(=O)OCCCN(CC)CC] |
| 16A | 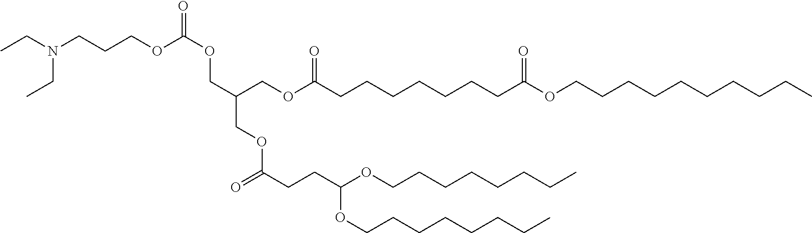 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 17A | 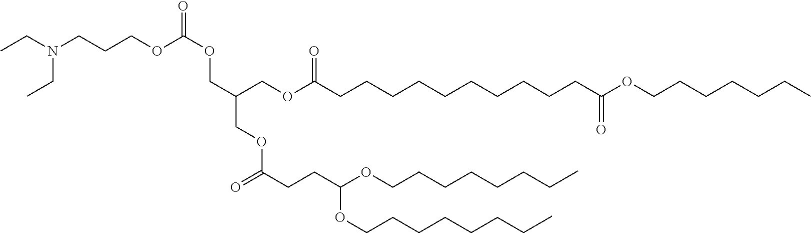 [SMILES: CCCCCCCCOC(CCC(=O)OCC(COC(=O)CCCCCCCCCCC(=O)OCCCCCCC)COC(=O)OCCCN(CC)CC)OCCCCCCCC] |
| 18A | 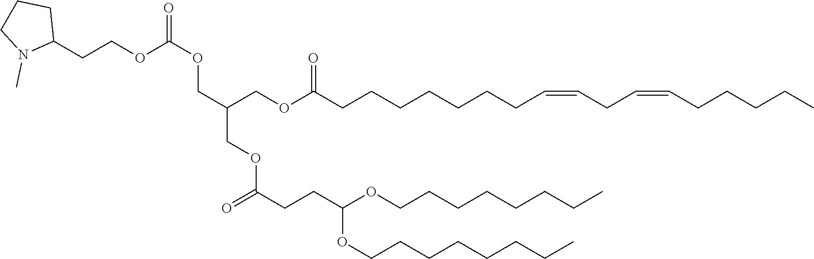 [SMILES: CCCCC/C=C\C/C=C\CCCCCCCC(=O)OCC(COC(=O)CCC(OCCCCCCCC)OCCCCCCCC)COC(=O)OCCC1CCCN1C] |
| 19A | 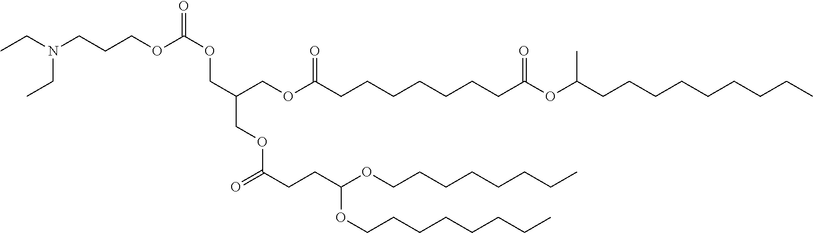 [SMILES: CCCCCCCCCC(C)OC(=O)CCCCCCCC(=O)OCC(COC(=O)CCC(OCCCCCCCC)OCCCCCCCC)COC(=O)OCCCN(CC)CC] |

or a salt thereof, such as a pharmaceutically acceptable salt thereof.

In some embodiments, the amine lipid is Lipid D, which is nonyl 8-((7,7-bis(octyloxy)heptyl)(2-hydroxyethyl)amino)octanoate:

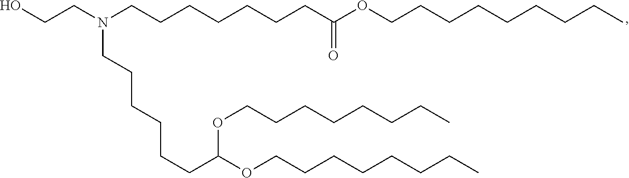 [SMILES: CCCCCCCCCOC(=O)CCCCCCCN(CCO)CCCCCCC(OCCCCCCCC)OCCCCCCCC]

or a salt thereof.

Lipid D may be synthesized according to WO2020072605 and Mol. Ther. 2018, 26(6), 1509-1519 (“Sabnis”), which are incorporated by reference in their entireties. In some embodiments, the amine lipid Lipid D, or an amine lipid provided in WO2020072605, which is hereby incorporated by reference.

In some embodiments, the amine lipid is a compound having a structure of Formula IB.

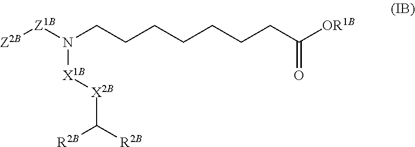

wherein
X^{1B} is C₆₋₇ alkylene;
X^{2B} is

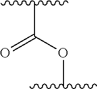 [SMILES: *OC(*)=O]

or absent, provided that if X^{2B} is

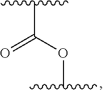 [SMILES: *OC(*)=O]

R^{2B} is not alkoxy;
Z^{1B} is C₂₋₃ alkylene;
Z^{2B} is selected from —OH, —NHC(═O)OCH₃, and —NHS(═O)₂CH₃;
R^{1B} is C₇₋₉ unbranched alkyl; and
each R^{2B} is independently C₈ alkyl or C₈ alkoxy;
or a salt thereof

In some embodiments, the amine lipid is a compound of Formula (IIB)

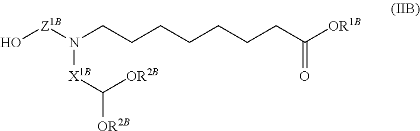

wherein
X^{1B} is C₆₋₇ alkylene;
Z^{1B} is C₂₋₃ alkylene;
R^{1B} is C₇₋₉ unbranched alkyl; and
each R^{2B} is C₈ alkyl;
or a salt thereof.

In certain embodiments, X^{1B} is C₆ alkylene. In other embodiments, X^{1B} is C₇ alkylene.

In certain embodiments, Z^{1B} is a direct bond and R^{5B} and R^{6B} are each C₈ alkoxy. In other embodiments, Z^{1B} is C₃ alkylene and R^{5B} and R^{6B} are each C₆ alkyl.

In certain embodiments, X^{2B} is

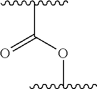 [SMILES: *OC(*)=O]

and R^{2B} is not alkoxy. In other embodiments, X^{2B} is absent.

In certain embodiments, Z^{1B} is C₂ alkylene; In other embodiments, Z^{1B} is C₃ alkylene.

In certain embodiments, Z^{2B} is —OH. In other embodiments, Z^{2B} is —NHC(═O)OCH₃. In other embodiments, Z^{2B} is —NHS(═O)₂CH₃.

In certain embodiments, R^{1B} is C₇ unbranched alkylene. In other embodiments, R^{1B} is C₈ branched or unbranched alkylene. In other embodiments, R^{1B} is C₉ branched or unbranched alkylene.

In certain embodiments, the amine lipid is a salt.

Representative compounds of Formula (IB) include:

| Compound | |
|---|---|
| Number | Compound |
| 1B | 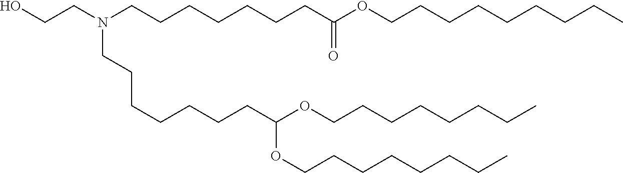 [SMILES: CCCCCCCCCOC(=O)CCCCCCCN(CCO)CCCCCCCC(OCCCCCCCC)OCCCCCCCC] |
| 2B | 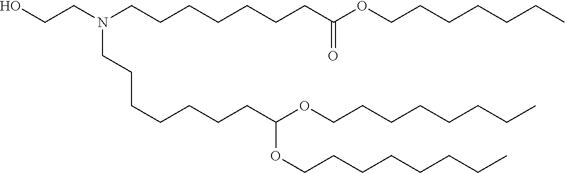 [SMILES: CCCCCCCCOC(CCCCCCCN(CCO)CCCCCCCC(=O)OCCCCCCC)OCCCCCCCC] |
| 3B | 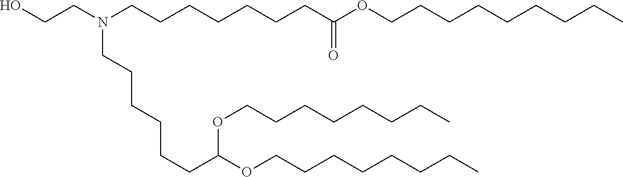 [SMILES: CCCCCCCCCOC(=O)CCCCCCCN(CCO)CCCCCCC(OCCCCCCCC)OCCCCCCCC] |
| 4B | 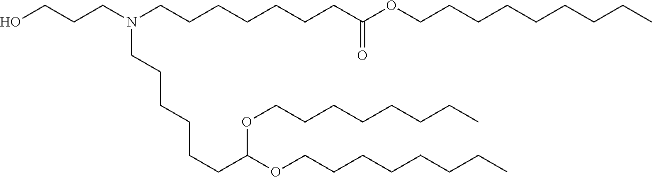 [SMILES: CCCCCCCCCOC(=O)CCCCCCCN(CCCO)CCCCCCC(OCCCCCCCC)OCCCCCCCC] |
| 5B | 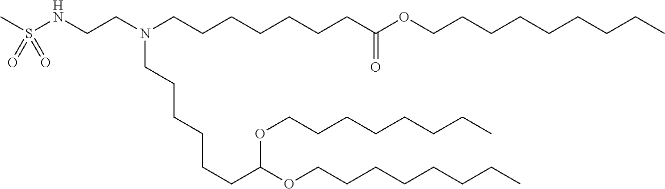 [SMILES: CCCCCCCCCOC(=O)CCCCCCCN(CCCCCCC(OCCCCCCCC)OCCCCCCCC)CCNS(C)(=O)=O] |
| 6B | 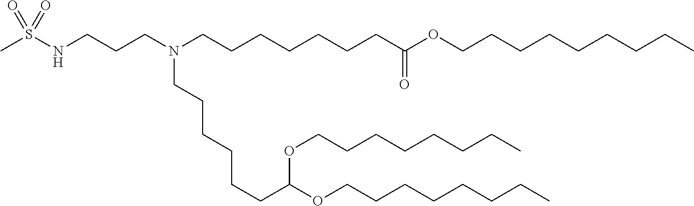 [SMILES: CCCCCCCCCOC(=O)CCCCCCCN(CCCCCCC(OCCCCCCCC)OCCCCCCCC)CCCNS(C)(=O)=O] |
| 7B | 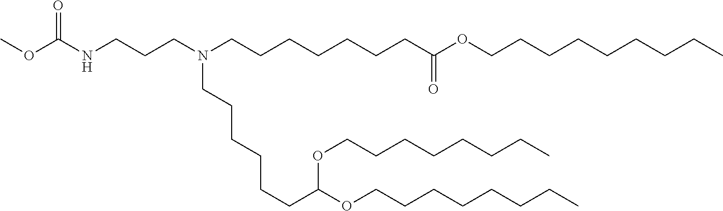 [SMILES: CCCCCCCCCOC(=O)CCCCCCCN(CCCCCCC(OCCCCCCCC)OCCCCCCCC)CCCNC(=O)OC] |

or a salt thereof, such as a pharmaceutically acceptable salt thereof.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g., an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO 2020/219876 (e.g., at pp. 13-33, 66-87), WO 2020/118041, WO 2020/072605 (e.g., at pp. 5-12, 21-29, 61-68, WO 2019/067992, WO 2017/173054, WO 2015/095340, and WO 2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of LNPs of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., to tumors. See, e.g., WO2014/136086.

### B. Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (IV), wherein n is 45, meaning that the number averaged degree of polymerization comprises about 45 subunits

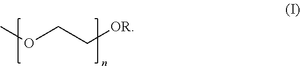

However, other PEG embodiments known in the art may be used, including, e.g., those where the number-averaged degree of polymerization comprises about 23 subunits (n=23), or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG catalog #GM-020 from NOF, Tokyo, Japan), such as e.g., 1,2-dimyristoyl-rac-glycero-3-methylpolyoxyethylene glycol 2000 (PEG2k-DMG), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMPE) (cat. #880150P from Avanti Polar Lipids, Alabaster, Alabama, USA), 1,2-distearoyl-sn-glycero-3-phosphoethanolamine-N-[methoxy(polyethylene glycol)-2000](PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000](PEG2k-DSA). In one embodiment, the PEG lipid may be 1,2-dimyristoyl-rac-glycero-3-methylpolyoxyethylene glycol 2000. In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In some embodiments, the PEG lipid includes a glycerol group. In some embodiments, the PEG lipid includes a dimyristoylglycerol (DMG) group. In some embodiments, the PEG lipid comprises PEG-2k. In some embodiments, the PEG lipid is a PEG-DMG. In some embodiments, the PEG lipid is a PEG-2k-DMG. In some embodiments, the PEG lipid is 1,2-dimyristoyl-rac-glycero-3-methoxypolyethylene glycol2000. In some embodiments, the PEG-2k-DMG is 1,2-dimyristoyl-rac-glycero-3-methoxypolyethylene glycol-2000.

### C. Lipid Nanoparticles (LNPs)

The LNP may contain (i) a biodegradable lipid, (ii) an optional neutral lipid, (iii) a helper lipid, and (iv) a stealth lipid, such as a PEG lipid. The lipid nucleic acid assembly may contain a biodegradable lipid and one or more of a neutral lipid, a helper lipid, and a stealth lipid, such as a PEG lipid.

The lipid nucleic acid assembly may contain (i) an amine lipid for encapsulation and for endosomal escape, (ii) a neutral lipid for stabilization, (iii) a helper lipid, also for stabilization, and (iv) a stealth lipid, such as a PEG lipid. The lipid nucleic acid assembly may contain an amine lipid and one or more of a neutral lipid, a helper lipid, also for stabilization, and a stealth lipid, such as a PEG lipid.

An LNP may comprise a nucleic acid, e.g., an RNA, component that includes one or more of an RNA-guided DNA-binding agent, a Cas nuclease mRNA, a Class 2 Cas nuclease mRNA, a Cas9 mRNA, and a gRNA. In some embodiments, a LNP may include a Class 2 Cas nuclease and a gRNA as the RNA component. In some embodiments, an LNP may comprise the RNA component, an amine lipid, a helper lipid, a neutral lipid, and a stealth lipid. In certain LNPs, the helper lipid is cholesterol. In other compositions, the neutral lipid is DSPC. In additional embodiments, the stealth lipid is PEG2k-DMG or PEG2k-C11. In some embodiments, the LNP comprises Lipid A or an equivalent of Lipid A; a helper lipid; a neutral lipid; a stealth lipid; and an RNA such as a gRNA. In some embodiments, the LNP comprises Lipid A or an equivalent of Lipid A; a helper lipid; a stealth lipid; and an RNA such as a gRNA. In some compositions, the amine lipid is Lipid A. In some compositions, the amine lipid is Lipid A or an acetal analog thereof, the helper lipid is cholesterol; the neutral lipid is DSPC; and the stealth lipid is PEG2k-DMG.

In some embodiments, lipid compositions are described according to the respective molar ratios of the component lipids in the formulation. Embodiments of the present disclosure provide lipid compositions described according to the respective molar ratios of the component lipids in the formulation. In one embodiment, the mol % of the amine lipid may be from about 30 mol % to about 60 mol %. In one embodiment, the mol % of the amine lipid may be from about 40 mol % to about 60 mol %. In one embodiment, the mol % of the amine lipid may be from about 45 mol % to about 60 mol %. In one embodiment, the mol % of the amine lipid may be from about 50 mol % to about 60 mol %. In one embodiment, the mol % of the amine lipid may be from about 55 mol % to about 60 mol %. In one embodiment, the mol % of the amine lipid may be from about 50 mol % to about 55 mol %. In one embodiment, the mol % of the amine lipid may be about 50 mol %. In one embodiment, the mol % of the amine lipid may be about 55 mol %. In some embodiments, the amine lipid mol % of the lipid nucleic acid assembly batch will be ±30%, ±25%, ±20%, ±15%, ±10%, ±5%, or 2.5% of the target mol %. In some embodiments, the amine lipid mol % of the lipid nucleic acid assembly batch will be ±4 mol %, ±3 mol %, ±2 mol %, ±1.5 mol %, ±1 mol %, ±0.5 mol %, or ±0.25 mol % of the target mol %. All mol % numbers are given as a fraction of the lipid component of the LNPs. In some embodiments, lipid nucleic acid assembly inter-lot variability of the amine lipid mol % will be less than 15%, less than 10% or less than 5%.

In one embodiment, the mol % of the neutral lipid may be from about 5 mol % to about 15 mol %. In one embodiment, the mol % of the neutral lipid may be from about 7 mol % to about 12 mol %. In one embodiment, the mol % of the neutral lipid may be about 9 mol %. In some embodiments, the neutral lipid mol % of the lipid nucleic acid assembly batch will be ±30%, ±25%, ±20%, ±15%, ±10%, ±5%, or 2.5% of the target neutral lipid mol %. In some embodiments, lipid nucleic acid assembly inter-lot variability will be less than 15%, less than 10% or less than 5%.

In one embodiment, the mol % of the helper lipid may be from about 20 mol % to about 60 mol %. In one embodiment, the mol % of the helper lipid may be from about 25 mol % to about 55 mol %. In one embodiment, the mol % of the helper lipid may be from about 25 mol % to about 50 mol %. In one embodiment, the mol % of the helper lipid may be from about 25 mol % to about 40 mol %. In one embodiment, the mol % of the helper lipid may be from about 30 mol % to about 50 mol %. In one embodiment, the mol % of the helper lipid may be from about 30 mol % to about 40 mol %. In one embodiment, the mol % of the helper lipid is adjusted based on amine lipid, neutral lipid, and PEG lipid concentrations to bring the lipid component to 100 mol %. In some embodiments, the helper mol % of the lipid nucleic acid assembly batch will be ±30%, ±25%, ±20%, ±15%, ±10%, ±5%, or 2.5% of the target mol %. In some embodiments, lipid nucleic acid assembly inter-lot variability will be less than 15%, less than 10% or less than 5%.

In one embodiment, the mol % of the PEG lipid may be from about 1 mol % to about 10 mol %. In one embodiment, the mol % of the PEG lipid may be from about 2 mol % to about 10 mol %. In one embodiment, the mol % of the PEG lipid may be from about 1 mol % to about 3 mol %. In one embodiment, the mol % of the PEG lipid may be from about 2 mol % to about 4 mol %. In one embodiment, the mol % of the PEG lipid may be from about 1.5 mol % to about 2 mol %. In one embodiment, the mol % of the PEG lipid may be from about 2.5 mol % to about 4 mol %. In one embodiment, the mol % of the PEG lipid may be about 3 mol %. In one embodiment, the mol % of the PEG lipid may be about 2.5 mol %. In one embodiment, the mol % of the PEG lipid may be about 2 mol %. In one embodiment, the mol % of the PEG lipid may be about 1.5 mol %. In some embodiments, the PEG lipid mol % of the lipid nucleic acid assembly batch will be ±30%, ±25%, ±20%, ±15%, ±10%, ±5%, or ±2.5% of the target PEG lipid mol %. In some embodiments, LNP, e.g., the LNP composition, inter-lot variability will be less than 15%, less than 10% or less than 5%.

Embodiments of the present disclosure provide LNP compositions, for example, LNP compositions comprising an ionizable lipid (e.g., Lipid A or one of its analogs), a helper lipid, a helper lipid, and a PEG lipid, described according to the respective molar ratios of the component lipids in the formulation. In certain embodiments, the amount of the ionizable lipid is from about 25 mol % to about 45 mol %; the amount of the neutral lipid is from about 10 mol % to about 30 mol %; the amount of the helper lipid is from about 25 mol % to about 65 mol %; and the amount of the PEG lipid is from about 1.5 mol % to about 3.5 mol %. In certain embodiments, the amount of the ionizable lipid is from about 29-44 mol % of the lipid component; the amount of the neutral lipid is from about 11-28 mol % of the lipid component; the amount of the helper lipid is from about 28-55 mol % of the lipid component; and the amount of the PEG lipid is from about 2.3-3.5 mol % of the lipid component. In certain embodiments, the amount of the ionizable lipid is from about 29-38 mol % of the lipid component; the amount of the neutral lipid is from about 11-20 mol % of the lipid component; the amount of the helper lipid is from about 43-55 mol % of the lipid component; and the amount of the PEG lipid is from about 2.3-2.7 mol % of the lipid component. In certain embodiments, the amount of the ionizable lipid is from about 25-34 mol % of the lipid component; the amount of the neutral lipid is from about 10-20 mol % of the lipid component; the amount of the helper lipid is from about 45-65 mol % of the lipid component; and the amount of the PEG lipid is from about 2.5-3.5 mol % of the lipid component. In certain embodiments, the ionizable lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component. In certain embodiments, the ionizable lipid is about 33 mol % of the lipid component; the amount of the neutral lipid is about 15 mol % of the lipid component; the amount of the helper lipid is about 49 mol % of the lipid component; and the amount of the PEG lipid is about 3 mol % of the lipid component. In certain embodiments, the amount of the ionizable lipid is about 32.9 mol % of the lipid component; the amount of the neutral lipid is about 15.2 mol % of the lipid component; the amount of the helper lipid is about 49.2 mol % of the lipid component; and the amount of the PEG lipid is about 2.7 mol % of the lipid component.

In certain embodiments, the amount of the ionizable lipid (e.g., Lipid A or one of its analogs) is about 20-50 mol %, about 25-34 mol %, about 25-38 mol %, about 25-45 mol %, about 29-38 mol %, about 29-43 mol %, about 29-34 mol %, about 30-34 mol %, about 30-38 mol %, about 30-43 mol %, about 30-43 mol %, or about 33 mol %. In certain embodiments, the amount of the neutral lipid is about 10-30 mol %, about 11-30 mol %, about 11-20 mol %, about 13-17 mol %, or about 15 mol %. In certain embodiments, the amount of the helper lipid is about 35-50 mol %, about 35-65 mol %, about 35-55 mol %, about 38-50 mol %, about 38-55 mol %, about 38-65 mol %, about 40-50 mol %, about 40-65 mol %, about 43-65 mol %, about 43-55 mol %, or about 49 mol %. In certain embodiments, the amount of the PEG lipid is about 1.5-3.5 mol %, about 2.0-2.7 mol %, about 2.0-3.5 mol %, about 2.3-3.5 mol %, about 2.3-2.7 mol %, about 2.5-3.5 mol %, about 2.5-2.7 mol %, about 2.9-3.5 mol %, or about 2.7 mol %.

Other embodiments of the present disclosure provide LNP compositions, for example, LNP compositions comprising an ionizable lipid (e.g., Lipid D or one of its analogs), a helper lipid, a helper lipid, and a PEG lipid, described according to the respective molar ratios of the component lipids in the formulation. In certain embodiments, the amount of the ionizable lipid is from about 25 mol % to about 50 mol %; the amount of the neutral lipid is from about 7 mol % to about 25 mol %; the amount of the helper lipid is from about 39 mol % to about 65 mol %; and the amount of the PEG lipid is from about 0.5 mol % to about 1.8 mol %. In certain embodiments, the amount of the ionizable lipid is from about 27-40 mol % of the lipid component; the amount of the neutral lipid is from about 10-20 mol % of the lipid component; the amount of the helper lipid is from about 50-60 mol % of the lipid component; and the amount of the PEG lipid is from about 0.9-1.6 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component. In certain embodiments, the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component. In certain embodiments, the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In certain embodiments, the amount of the ionizable lipid (e.g., Lipid D or one of its analogs) is about 20-55 mol %, about 20-45 mol %, about 20-40 mol %, about 27-40 mol %, about 27-45 mol %, about 27-55 mol %, about 30-40 mol %, about 30-45 mol %, about 30-55 mol %, about 30 mol %, about 40 mol %, or about 50 mol %. In certain embodiments, the amount of the neutral lipid is about 7-25 mol %, about 10-25 mol %, about 10-20 mol %, about 15-20 mol %, about 8-15 mol %, about 10-15 mol %, about 10 mol %, or about 15 mol %. In certain embodiments, the amount of the helper lipid is about 39-65 mol %, about 39-59 mol %, about 40-60 mol %, about 40-65 mol %, about 40-59 mol %, about 43-65 mol %, about 43-60 mol %, about 43-59 mol %, or about 50-65 mol %, about 50-59 mol %, about 59 mol %, or about 43.5 mol %. In certain embodiments, the amount of the PEG lipid is about 0.5-1.8 mol %, about 0.8-1.6 mol %, about 0.8-1.5 mol %, 0.9-1.8 mol %, about 0.9-1.6 mol %, about 0.9-1.5 mol %, 1-1.8 mol %, about 1-1.6 mol %, about 1-1.5 mol %, about 1 mol %, or about 1.5 mol %.

In some embodiments, the cargo includes an mRNA encoding an RNA-guided DNA-binding agent (e.g., a Cas nuclease, a Class 2 Cas nuclease, or Cas9), or a gRNA or a nucleic acid encoding a gRNA, or a combination of mRNA and gRNA. In one embodiment, a LNP may comprise a Lipid A or its equivalents, or an amine lipid as provided in WO2020219876; or Lipid D or an amine lipid provided in WO2020/072605. In some aspects, the amine lipid is Lipid A, or Lipid D. In some aspects, the amine lipid is a Lipid A equivalent, e.g. an analog of Lipid A, or an amine lipid provided in WO2020/219876. In certain aspects, the amine lipid is an acetal analog of Lipid A, optionally, an amine lipid provided in WO2020/219876. In some aspects, the amine lipid is a Lipid D or an amine lipid found in in W2020072605. In various embodiments, a LNP comprises an amine lipid, a neutral lipid, a helper lipid, and a PEG lipid. In some embodiments, the helper lipid is cholesterol. In some embodiments, the neutral lipid is DSPC. In specific embodiments, PEG lipid is PEG2k-DMG. In some embodiments, a LNP may comprise a Lipid A, a helper lipid, a neutral lipid, and a PEG lipid. In some embodiments, a LNP comprises an amine lipid, DSPC, cholesterol, and a PEG lipid. In some embodiments, the LNP comprises a PEG lipid comprising DMG. In some embodiments, the amine lipid is selected from Lipid A, and an equivalent of Lipid A, including an acetal analog of Lipid A, or an amine lipid provided in WO2020/219876; or Lipid D or an amine lipid provided in WO2020/072605. In additional embodiments, a LNP comprises Lipid A, cholesterol, DSPC, and PEG2k-DMG. In additional embodiments, a LNP comprises Lipid D, cholesterol, DSPC, and PEG2k-DMG.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, a LNP may comprise a lipid component that comprises an amine lipid, a helper lipid, a neutral lipid, and a helper lipid; and a nucleic acid component, wherein the N/P ratio is about 3 to 10. In some embodiments, the LNPs comprise molar ratios of an amine lipid to RNA/DNA phosphate (N:P) of about 4.5, 5.0, 5.5, 6.0, or 6.5. In some embodiments, a LNP may comprise a lipid component that comprises an amine lipid, a helper lipid, a neutral lipid, and a helper lipid; and an RNA component, wherein the N/P ratio is about 3 to 10. In one embodiment, the N/P ratio may about 5-7. In one embodiment, the N/P ratio may about 4.5-8. In one embodiment, the N/P ratio may about 6. In one embodiment, the N/P ratio may be 6±1. In one embodiment, the N/P ratio may about 6±0.5. In some embodiments, the N/P ratio will be 30%, ±25%, ±20%, ±15%, ±10%, ±5%, or ±2.5% of the target N/P ratio. In some embodiments, lipid nucleic acid assembly inter-lot variability will be less than 15%, less than 10% or less than 5%.

In some embodiments, the lipid nucleic acid assembly comprises an RNA component, which may comprise an mRNA, such as an mRNA encoding a Cas nuclease. In one embodiment, RNA component may comprise a Cas9 mRNA. In some compositions comprising an mRNA encoding a Cas nuclease, the lipid nucleic acid assembly further comprises a gRNA nucleic acid, such as a gRNA. In some embodiments, the RNA component comprises a Cas nuclease mRNA and a gRNA. In some embodiments, the RNA component comprises a Class 2 Cas nuclease mRNA and a gRNA.

In some embodiments, a LNP may comprise an mRNA encoding a Cas nuclease such as a Class 2 Cas nuclease, an amine lipid, a helper lipid, a neutral lipid, and a PEG lipid. In certain LNPs comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, the PEG lipid is PEG2k-DMG or PEG2k-C11. In specific compositions comprising an mRNA encoding a Cas nuclease such as a Class 2 Cas nuclease, the amine lipid is selected from Lipid A and its equivalents, such as an acetal analog of Lipid A, or amine lipids provided in WO2020/219876; or Lipid D or amine lipids provided in WO2020/072605.

In some embodiments, an LNP may comprise a gRNA. In some embodiments, a LNP may comprise an amine lipid, a gRNA, a helper lipid, a neutral lipid, and a PEG lipid. In certain LNPs comprising a gRNA, the helper lipid is cholesterol. In some compositions comprising a gRNA, the neutral lipid is DSPC. In additional embodiments comprising a gRNA, the PEG lipid is PEG2k-DMG or PEG2k-C11. In some embodiments, the amine lipid is selected from Lipid A and its equivalents, such as an acetal analog of Lipid A, or amine lipids provided in WO2020/219876 and their equivalents; or Lipid D or amine lipids provided in WO2020/072605 and their equivalents.

In one embodiment, a LNP may comprise an sgRNA. In one embodiment, a LNP may comprise a Cas9 sgRNA. In one embodiment, a LNP may comprise a Cpf1 sgRNA. In some compositions comprising an sgRNA, the lipid nucleic acid assembly includes an amine lipid, a helper lipid, a neutral lipid, and a PEG lipid. In certain compositions comprising an sgRNA, the helper lipid is cholesterol. In other compositions comprising an sgRNA, the neutral lipid is DSPC. In additional embodiments comprising an sgRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D or amine lipids provided in WO2020/072605.

In some embodiments, a LNP comprises an mRNA encoding a Cas nuclease and a gRNA, which may be an sgRNA. In one embodiment, a LNP may comprise an amine lipid, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D or amine lipids provided in WO2020/072605.

In some embodiments, the LNPs include a Cas nuclease mRNA, such as a Class 2 Cas mRNA and at least one gRNA. In some embodiments, the LNP includes a ratio of gRNA to Cas nuclease mRNA, such as Class 2 Cas nuclease mRNA from about 25:1 to about 1:25 wt/wt. In some embodiments, the lipid nucleic acid assembly formulation includes a ratio of gRNA to Cas nuclease mRNA, such as Class 2 Cas nuclease mRNA from about 10:1 to about 1:10. In some embodiments, the lipid nucleic acid assembly formulation includes a ratio of gRNA to Cas nuclease mRNA, such as Class 2 Cas nuclease mRNA from about 8:1 to about 1:8. As measured herein, the ratios are by weight. In some embodiments, the lipid nucleic acid assembly formulation includes a ratio of gRNA to Cas nuclease mRNA, such as Class 2 Cas mRNA from about 5:1 to about 1:5. In some embodiments, ratio range is about 3:1 to 1:3, about 2:1 to 1:2, about 5:1 to 1:2, about 5:1 to 1:1, about 3:1 to 1:2, about 3:1 to 1:1, about 3:1, about 2:1 to 1:1. In some embodiments, the gRNA to mRNA ratio is about 3:1 or about 2:1. In some embodiments the ratio of gRNA to Cas nuclease mRNA, such as Class 2 Cas nuclease is about 1:1. In some embodiments the ratio of gRNA to Cas nuclease mRNA, such as Class 2 Cas nuclease is about 1:2. The ratio may be about 25:1, 10:1, 5:1, 3:1, 2:1, 1:1, 1:2, 1:3, 1:5, 1:10, or 1:25.

The LNPs disclosed herein may include a template nucleic acid. The template nucleic acid may be co-formulated with an mRNA encoding a Cas nuclease, such as a Class 2 Cas nuclease mRNA. In some embodiments, the template nucleic acid may be co-formulated with a guide RNA. In some embodiments, the template nucleic acid may be co-formulated with both an mRNA encoding a Cas nuclease and a guide RNA. In some embodiments, the template nucleic acid may be formulated separately from an mRNA encoding a Cas nuclease or a guide RNA. The template nucleic acid may be delivered with, or separately from the LNPs. In some embodiments, the template nucleic acid may be single- or double-stranded, depending on the desired repair mechanism. The template may have regions of homology to the target DNA, or to sequences adjacent to the target DNA.

In some embodiments, lipid nucleic acid assemblies are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of lipid nucleic acid assemblies, may be used. In some embodiments, a buffer is used to maintain the pH of the composition comprising lipid nucleic acid assemblies at or above pH 6.5. In some embodiments, a buffer is used to maintain the pH of the composition comprising lipid nucleic acid assemblies at or above pH 7.0. In some embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In some embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In some embodiments, the LNP may include about 1, 2, 3, 4, 5, 6, 7, 8, 9, or 10% cryoprotectant. In some embodiments, the LNP may include about 1, 2, 3, 4, 5, 6, 7, 8, 9, or 10% sucrose. In some embodiments, the LNP may include a buffer. In some embodiments, the buffer may comprise a phosphate buffer (PBS), a Tris buffer, a citrate buffer, and mixtures thereof. In some exemplary embodiments, the buffer comprises NaCl. In some embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNPs contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, or RNA and lipid concentrations may be varied. Lipid nucleic acid assemblies or LNPs may be concentrated or purified, e.g., via dialysis, tangential flow filtration, or chromatography. The lipid nucleic acid assemblies may be stored as a suspension, an emulsion, or a lyophilized powder, for example. In some embodiments, a LNP is stored at 2-8° C., in certain aspects, the LNPs are stored at room temperature. In additional embodiments, a LNP is stored frozen, for example at −20° C. or −80° C. In other embodiments, a LNP is stored at a temperature ranging from about 0° C. to about −80° C. Frozen LNPs may be thawed before use, for example on ice, at 4° C., at room temperature, or at 25° C. Frozen LNPs may be maintained at various temperatures, for example on ice, at 4° C., at room temperature, at 25° C., or at 37° C.

In some embodiments, the concentration of the LNPs in the LNP composition is about 1-10 μg/mL, about 2-10 ug/mL, about 2.5-10 ug/mL, about 1-5 ug/mL, about 2-5 ug/mL, about 2.5-5 ug/mL, about 0.04 ug/mL, about 0.08 ug/mL, about 0.16 ug/mL, about 0.25 ug/mL, about 0.63 ug/mL, about 1.25 ug/mL, about 2.5 ug/mL, or about 5 μg/mL.

In some embodiments, the LNP comprises a stealth lipid, optionally wherein:
(i) the LNP comprises a lipid component and the lipid component comprises: about 50-60 mol % amine lipid such as Lipid A or Lipid D, about 8-10 mol % neutral lipid; and about 2.5-4 mol % stealth lipid (e.g., a PEG lipid), wherein the remainder of the lipid component is helper lipid, and wherein the N/P ratio of the LNP is about 6;
(ii) the LNP comprises about 50-60 mol % amine lipid such as Lipid A or Lipid D; about 27-39.5 mol % helper lipid; about 8-10 mol % neutral lipid; and about 2.5-4 mol % stealth lipid (e.g., a PEG lipid), wherein the N/P ratio of the LNP is about 5-7 (e.g., about 6);
(iii) the LNP comprises a lipid component and the lipid component comprises: about 50-60 mol % amine lipid such as Lipid A or Lipid D; about 5-15 mol % neutral lipid; and about 2.5-4 mol % stealth lipid (e.g., a PEG lipid), wherein the remainder of the lipid component is helper lipid, and wherein the N/P ratio of the LNP is about 3-10;
(iv) the LNP comprises a lipid component and the lipid component comprises: about 40-60 mol % amine lipid such as Lipid A or Lipid D; about 5-15 mol % neutral lipid; and about 2.5-4 mol % stealth lipid (e.g., a PEG lipid), wherein the remainder of the lipid component is helper lipid, and wherein the N/P ratio of the LNP is about 6;
(v) the LNP comprises a lipid component and the lipid component comprises: about 50-60 mol % amine lipid such as Lipid A or Lipid D; about 5-15 mol % neutral lipid; and about 1.5-10 mol % stealth lipid (e.g., a PEG lipid), wherein the remainder of the lipid component is helper lipid, and wherein the N/P ratio of the LNP is about 6;
(vi) the LNP comprises a lipid component and the lipid component comprises: about 40-60 mol % amine lipid such as Lipid A or Lipid D; about 0-10 mol % neutral lipid; and about 1.5-10 mol % stealth lipid (e.g., a PEG lipid), wherein the remainder of the lipid component is helper lipid, and wherein the N/P ratio of the LNP is about 3-10;
(vii) the LNP comprises a lipid component and the lipid component comprises: about 40-60 mol % amine lipid such as Lipid A or Lipid D; less than about 1 mol % neutral lipid; and about 1.5-10 mol % stealth lipid (e.g., a PEG lipid), wherein the remainder of the lipid component is helper lipid, and wherein the N/P ratio of the LNP is about 3-10;
(viii) the LNP comprises a lipid component and the lipid component comprises: about 40-60 mol % amine lipid such as Lipid A or Lipid D; and about 1.5-10 mol % stealth lipid (e.g., a PEG lipid), wherein the remainder of the lipid component is helper lipid, wherein the N/P ratio of the LNP composition is about 3-10, and wherein the LNP is essentially free of or free of neutral phospholipid; or
(ix) the LNP comprises a lipid component and the lipid component comprises: about 50-60 mol % amine lipid such as Lipid A or Lipid D; about 8-10 mol-% neutral lipid; and about 2.5-4 mol % stealth lipid (e.g., a PEG lipid), wherein the remainder of the lipid component is helper lipid, and wherein the N/P ratio of the LNP is about 3-7.

In some embodiments, the LNP comprises a lipid component and the lipid component comprises: about 50 mol % amine lipid such as Lipid A or Lipid D; about 9 mol % neutral lipid such as DSPC; about 3 mol % of stealth lipid such as a PEG lipid, such as PEG2k-DMG, and the remainder of the lipid component is helper lipid such as cholesterol wherein the N/P ratio of the LNP is about 6.

In some embodiments, the LNP comprises a lipid component and the lipid component comprises: about 50 mol % Lipid A; about 9 mol % DSPC; about 3 mol % of PEG2k-DMG, and the remainder of the lipid component is cholesterol wherein the N/P ratio of the LNP is about 6.

In some embodiments, the LNP comprises a lipid component and the lipid component comprises: about 35 mol % Lipid A; about 15 mol % neutral lipid; about 47.5 mol % helper lipid; and about 2.5 mol % stealth lipid (e.g., PEG lipid), and wherein the N/P ratio of the LNP composition is about 3-7.

In some embodiments, the LNP comprises a lipid component and the lipid component comprises: about 35 mol % Lipid D; about 15 mol % neutral lipid; about 47.5 mol % helper lipid; and about 2.5 mol % stealth lipid (e.g., PEG lipid), and wherein the N/P ratio of the LNP composition is about 3-7.

In some embodiments, the LNP comprises a lipid component and the lipid component comprises: about 25-45 mol % amine lipid, such as Lipid A; about 10-30 mol % neutral lipid; about 25-65 mol % helper lipid; and about 1.5-3.5 mol % stealth lipid (e.g., PEG lipid), and wherein the N/P ratio of the LNP composition is about 3-7.

In some embodiments, the LNP comprises a lipid component, wherein:
a. the amount of the amine lipid is about 29-44 mol % of the lipid component; the amount of the neutral lipid is about 11-28 mol % of the lipid component; the amount of the helper lipid is about 28-55 mol % of the lipid component; and the amount of the PEG lipid is about 2.3-3.5 mol % of the lipid component
b. the amount of the amine lipid is about 29-38 mol % of the lipid component; the amount of the neutral lipid is about 11-20 mol % of the lipid component; the amount of the helper lipid is about 43-55 mol % of the lipid component; and the amount of the PEG lipid is about 2.3-2.7 mol % of the lipid component;
c. the amount of the amine lipid is about 25-34 mol % of the lipid component; the amount of the neutral lipid is about 10-20 mol % of the lipid component; the amount of the helper lipid is about 45-65 mol % of the lipid component; and the amount of the PEG lipid is about 2.5-3.5 mol % of the lipid component; or
d. the amount of the amine lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component.

In some embodiments, the LNP comprises a lipid component and the lipid component comprises: about 25-50 mol % amine lipid, such as Lipid D; about 7-25 mol % neutral lipid; about 39-65 mol % helper lipid; and about 0.5-1.8 mol % stealth lipid (e.g., PEG lipid), and wherein the N/P ratio of the LNP composition is about 3-7.

In some embodiments, the LNP comprises a lipid component wherein the amount of the amine lipid is about 30-45 mol % of the lipid component; or about 30-40 mol % of the lipid component; optionally about 30 mol %, 40 mol %, or 50 mol % of the lipid component. In some embodiments, the LNP comprises a lipid component wherein the amount of the neutral lipid is about 10-20 mol % of the lipid component; or about 10-15 mol % of the lipid component; optionally about 10 mol % or 15 mol % of the lipid component. In some embodiments, the LNP comprises a lipid component wherein the amount of the helper lipid is about 50-60 mol % of the lipid component; about 39-59 mol % of the lipid component; or about 43.5-59 mol % of the lipid component; optionally about 59 mol % of the lipid component; about 43.5 mol % of the lipid component; or about 39 mol % of the lipid component. In some embodiments, the LNP comprises a lipid component wherein the amount of the PEG lipid is about 0.9-1.6 mol % of the lipid component; or about 1-1.5 mol % of the lipid component; optionally about 1 mol % of the lipid component or about 1.5 mol % of the lipid component

In some embodiments, the LNP comprises a lipid component, wherein:
a. the amount of the ionizable lipid is about 27-40 mol % of the lipid component; the amount of the neutral lipid is about 10-20 mol % of the lipid component; the amount of the helper lipid is about 50-60 mol % of the lipid component; and the amount of the PEG lipid is about 0.9-1.6 mol % of the lipid component;
b. the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component;
c. the amount of the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component;
d. the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component; or
e. the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In some embodiments, the LNP has a diameter of about 1-250 nm, 10-200 nm, about 20-150 nm, about 50-150 nm, about 50-100 nm, about 50-120 nm, about 60-100 nm, about 75-150 nm, about 75-120 nm, or about 75-100 nm. In some embodiments, the LNP has a diameter of less than 100 nm. In some embodiments, the LNP composition comprises a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm. In some embodiments, the LNP has an average diameter of less than 100 nm.

In some embodiments, the LNP comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the LNP composition is about 3-10. In some embodiments, the LNP comprises: about 50-60 mol-% amine lipid; about 8-10 mol-% neutral lipid; and about 2.5-4 mol-% PEG lipid, wherein the remainder of the lipid component is helper lipid, and wherein the N/P ratio of the LNP composition is about 3-8. In some embodiments, the LNP comprises: about 50-60 mol-% amine lipid; about 5-15 mol-% DSPC; and about 2.5-4 mol-% PEG lipid, wherein the remainder of the lipid component is cholesterol, and wherein the N/P ratio of the LNP composition is 3-8±0.2.

In embodiments, the average diameter is a Z-average diameter. In certain embodiments, the Z-average diameter is measured by dynamic light scattering (DLS) using methods known in the art. For example, average particle size and polydispersity can be measured by dynamic light scattering (DLS) using a Malvern Zetasizer DLS instrument. LNP samples are diluted with PBS buffer prior to being measured by DLS. Z-average diameter and number average diameter along with a polydispersity index (pdi) can be determined. The Z average is the intensity weighted mean hydrodynamic size of the ensemble collection of particles. The number average is the particle number weighted mean hydrodynamic size of the ensemble collection of particles. A Malvern Zetasizer instrument can also be used to measure the zeta potential of the LNP using methods known in the art.

### D. Targeted LNPs

In certain embodiments, the LNP disclosed herein is a LNP that is capable of delivering diverse cargoes to a cell or cell population, e.g., a tissue or organ, of interest (herein referred to as a “targeted LNP”). Targeted LNPs may utilize various active targeting (governed by interactions between a targeting domain attached to the targeted LNP, for example by a chemical means, and targets associated with a cell or cell population), passive targeting (governed primarily by LNP size and charge), and endogenous targeting mechanisms. Accordingly, in some embodiments, the targeted LNP comprises one or more targeting domains that targets the LNP to the specific cell or cell population. Targeting domains of the present disclosure include, but are not limited to, antibodies, antibody fragments, proteins, peptides, and nucleic acids. Alternatively or additionally, in certain embodiments, the targeted LNP comprises one or more targeting lipid components. In some embodiments, the targeted LNP is targeted to one or more of brain, eye, muscle, liver, lung, spleen, and bone marrow. Such LNPs are further described in, e.g., Akinc, A., et al. Mol Ther. 2010 (July), 18(7), 1357-1364; Cheng, Q., et al. Nat. Nanotechnol. 2020 (April), 15(4), 313-320; Herrara-Barrera, M., et al. Sci. Adv. 9, eadd4623(2023); Kasiewicz, L. N., et al. Lipid nanoparticles incorporating a GalNAc ligand enable in vivo liver ANGPTL3 editing in wild-type and somatic LDLR knockout non-human primates. BioRXiv. 2021, 11.08.467731; Kularatne, R. N., et al. Pharmaceuticals. 2022, 15, 897; Li, Q., et al. ACS Chem Biol. 2020 (April), 15(4), 830-836; Sago, C. D., et al. J Am Chem Soc. 2018 (December), 140(49), 17095-17105; Tombacz, I., et al. Mol Ther. 2021 (November), 29(11), 3293-3304; Veiga, N., et al. Nat Commun. 2018, 9, 4493; Wang, X., et al. Nat. Protoc. 2023 (January), 18(1): 265-291; WO 2022/232514, WO2022204219, WO2022140252, the contents of each of which are herein incorporated by reference. Targeted LNPs include, but are not limited to, selective organ targeting (SORT) LNPs as described in Cheng, Q., et al., 2020. Nat. Nanotechnol. 2020 (April), 15(4), 313-320.

In certain embodiments, the targeted LNP comprises one or more targeting domains that targets the LNP to the specific cell or cell population. The targeting domain may comprise a nucleic acid, peptide, antibody, small molecule, glycan, sugar, hormone, and the like that targets the LNP to the cell or cell population. In certain embodiments, the LNP is capable of multivalent targeting, wherein the LNP comprises multiple targeting mechanisms described herein. In certain embodiments, the targeting domain of the LNP specifically binds to a target associated with a cell or cell population in need of cargo associated with the LNP composition disclosed herein. For example, the targeting domain may be chosen to recognize a ligand that acts as a cell surface marker on target cells or cell populations associated with a particular disease state. Such a target can be a protein, protein fragment, antigen, or other biomolecule that is associated with the targeted cell or cell population. In some embodiments, the targeting domain is an affinity ligand which specifically binds to a target. In certain embodiments, the target (e.g., an antigen) is associated with a cell or cell population in need of a treatment with the cargo associated with the LNP. In some embodiments, the targeting domain may be co-polymerized with the composition comprising the LNP. In some embodiments, the targeting domain may be covalently attached to the composition comprising the LNP, such as through a chemical reaction between the targeting domain and the LNP.

### 1. Peptide Targeting Domains

In one embodiment, the targeting domain of the disclosure comprises a peptide. In certain embodiments, the peptide targeting domain specifically binds to a target, e.g., on a cell or cell population of interest.

The peptide of the present disclosure may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The peptides of the disclosure may include unnatural amino acids formed by post-translational modification or by introducing unnatural amino acids during translation.

### 2. Nucleic Acid Targeting Domains

In one embodiment, the targeting domain of the disclosure comprises an isolated nucleic acid, including for example a DNA and an RNA. In certain embodiments, the nucleic acid targeting domain specifically binds to a target, e.g., on a cell or cell population of interest. For example, in one embodiment, the nucleic acid comprises a nucleotide sequence that specifically binds to a target of interest.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

### 3. Antibodies

In one embodiment, the targeting domain of the disclosure comprises an antibody, or antibody fragment. In certain embodiments, the antibody targeting domain specifically binds to a target, e.g., on a cell or cell population of interest. Such antibodies include polyclonal antibodies, monoclonal antibodies, Fab and single chain Fv (scFv) fragments thereof, VHH domains thereof, nanobodies, bispecific antibodies, heteroconjugates, human and humanized antibodies. The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmacokinetic activity may be generated in a bacterial expression system. Single chain Fv fragments show low immunogenicity.

In certain embodiments, the targeted LNP comprises a targeting lipid component. The LNPs disclosed herein may comprise a biodegradable lipid (i.e., an amine lipid or ionizable lipid), a neutral lipid, a helper lipid (e.g., cholesterol), and a stealth lipid (e.g., a PEG lipid). In some embodiments, wherein the LNP is a targeted LNP disclosed herein, the targeted LNP may comprise a biodegradable lipid, a neutral lipid, a helper lipid, and a stealth lipid, and further comprise a targeting lipid component that may result in alterations to LNP size or charge. This, in turn, may impact LNP uptake by different cells, cell populations, tissue types, and organ systems. For example, LNPs larger than 200 nm exhibit reduced hepatocyte targeting, likely because they are unable to fit through the comparatively narrower (˜100 nm) sinusoidal fenestration pores (Kularatne, R. N., et al. The Future of Tissue-Targeted Lipid Nanoparticle-Mediated Nucleic Acid Delivery. Pharmaceuticals. 2022, 15, 897. See also Wang, X., et al. Preparation of Selective Organ-Targeting (SORT) Lipid Nanoparticles (LNPs) Using Multiple Technical Methods for Tissue-Specific mRNA Delivery. Nat. Protoc. 2023 (January), 18(1): 265-291). Suitable ratios for the targeting lipid component relative to other components of the LNP may be prepared within the common knowledge in the art, for example, Cheng, et al., 2020. Nat. Nanotechnol. 2020 (April), 15(4), 313-320.

In some embodiments, the targeting lipid component is a permanently cationic lipid, an anionic lipid, a zwitterionic lipid, or an ionizable cationic lipid. In some embodiments, the permanently cationic lipid is 1,2-dioleoyl-3-trimethylammonium-propane (DOTAP), dimethyldioctadecylammonium (DDAB), or 1,2-dimyristoyl-sn-glycero-3-ethylphosphocholine (EPC). In some embodiments, the anionic lipid is 1,2-dimyristoyl-sn-glycero-3-phosphate (14PA) or sn-(3-oleoyl-2-hydroxy)-glycerol-1-phospho-sn-3′-(1′,2′-dioleoyl)-glycerol (18BMP). In some embodiments, the zwitterionic lipid is 2-((2,3-bis(oleoyloxy)propyl)dimethylammonio)ethyl ethyl phosphate (DOCPe) or 1,2-distearoyl-sn-glycero-3-phosphocholine (DPSC). In some embodiments, the ionizable cationic lipid is 1,2-dioleoyl-3-dimethylammonium-propane (DODAP) or C12-200.

In certain embodiments, the targeted LNP is targeted to one or more cell populations or tissues (e.g., brain, eye, muscle, liver, lung, spleen, bone marrow).

In some embodiments, the LNP is targeted to the brain. Exemplary cell or cell populations of interest include, but are not limited to, an astrocyte, an oligodendrocyte, an endothelial cell, a microglial cell, an ependymal cell, or a neuron.

In some embodiments, wherein the targeted LNP is targeted to the brain, the targeted LNP comprises a targeting lipid component. In some embodiments, wherein the targeted LNP is targeted to the brain, the targeted LNP comprises a targeting domain.

In some embodiments, the targeted LNP is targeted to the lung. In some embodiments, wherein the targeted LNP is targeted to the lung, the cell or cell population of interest comprises an airway epithelial cell such as a goblet cell, a ciliated cell, a clara cell, a neuroendocrine cell, a basal cell, an intermediate or parabasal cell, a serous cell, a brush cell, an oncocyte, a nonciliated columnar cell, or a metaplastic cell; an alveolar cell such as a type 1 or type 2 pneumocyte, or a cuboidal nonciliated cell; a bronchial salivary gland cell such as a serous cell, a mucous cell, or a ductal cell; an interstitial connective tissue cell such as a smooth muscle cell, a cartilage cell, a fibroblast, a myofibroblast, a meningothelioid cell of the minute meningothelioid nodules, an adipose cell, or a neural cell of the intrapulmonary nerves; a blood vessel-associated cell such as an endothelial cell, a smooth muscle cell, a fibroblast or myofibroblast, or a pericyte; a hematopoietic or lymphoid cell such as a lymphocyte, plasma cell, cell of the bronchial mucosal associated lymphoid tissue, megakaryocyte, macrophage, Langerhans cell, mast cell, eosinophil, neutrophil, or basophil; a pleural cell such as a mesothelial cell, a pleuripotent submesothelial fibroblast, an adipose cell of the intrapleural fat, an endothelial cell, a smooth muscle cell, or a fibroblast or myofibroblast; a stem cell; a perivascular epithelioid cell; a pluripotent epithelial stem cell; a meningothelioid cell; an endothelial progenitor cell; or a mucinous cell.

In some embodiments, wherein the targeted LNP is targeted to the lung, the targeted LNP comprises a targeting lipid component. In some embodiments, the targeting lipid component is a permanently cationic lipid such as 1,2-dioleoyl-3-trimethylammonium-propane (DOTAP), DDAB, or EPC. In some embodiments, wherein the targeted LNP is targeted to the lung, the targeted LNP comprises a targeting domain. In some embodiments, the targeting domain is Fab-C4, plasmalemma vesicle-associated protein (PV-1), or anti PECAM-1 antibody.

In some embodiments, the Targeted LNP is targeted to the spleen. In some embodiments, wherein the targeted LNP is targeted to the spleen, the cell or cell population of interest comprises a red pulp cell such as a fibroblast, reticular cell, macrophage, erythrocyte, granulocyte, circulating mononuclear cell, lymphocyte, hematopoietic cell, plasma cell, plasmablast, endothelial cell, erythroid cell, myeloid cell, megakaryocyte, or melanocyte; a white pulp cell such as a lymphocyte, macrophage, dendritic cell, plasma cell, reticular cell, or stromal cell; or a marginal zone cell such as a macrophage, an endothelial cell, a reticular fibroblast, dendritic cell, or lymphocyte; or a lymphoid-tissue inducer cell.

In some embodiments, wherein the targeted LNP is targeted to the spleen, the targeted LNP comprises a targeting lipid component. In some embodiments, the targeting lipid component is a negatively charged 1,2-dioleoyl-sn-glycero-3-phosphate (18PA). In some embodiments, the targeting lipid component is an anionic lipid such as 1,2-dimyristoyl-sn-glycero-3-phosphate (14PA) or sn-(3-oleoyl-2-hydroxy)-glycerol-1-phospho-sn-3′-(1′,2′-dioleoyl)-glycerol (18BMP). In some embodiments, the targeting lipid component is a zwitterionic lipid such as DOCPe or DSPC. In some embodiments, wherein the targeted LNP is targeted to the spleen, the targeted LNP comprises a targeting domain.

In some embodiments, the targeted LNP is targeted to the bone marrow. In some embodiments, wherein the targeted LNP is targeted to the bone marrow, the targeted LNP comprises a reticular cell, a periarteriolar cell, a Schwann cell, an osteoclast, an N-cadherin+ cell, an osteoblast, a megakaryocyte, an erythroblast, a hematopoietic stem cell, a granulocyte monocyte progenitor cell, an erythroid progenitor cell, a lympohoid progenitor cell, or a multipotent progenitor cell.

In some embodiments, wherein the targeted LNP is targeted to the bone marrow, the targeted LNP comprises a targeting lipid component. In some embodiments, wherein the targeted LNP is targeted to the bone marrow, the targeted LNP comprises a targeting domain. In some embodiments, wherein the targeted LNP is targeted to the bone marrow, the targeting domain is specific for a target selected from CD34, CD117, CD133, CD105, ABCG2, Bone morphogenetic protein receptor (BMPR), CD44, Sca-1, Thy-1, CD 133, alkaline phosphatase, and alpha-fetoprotein. In some embodiments, wherein the targeted LNP is targeted to the bone marrow, the targeted LNP comprises anti-CD29.

In some embodiments, the targeted LNP comprises a polynucleotide comprising an open reading frame (ORF) encoding a fusion protein, wherein the fusion protein comprises a first cleavase and a second cleavase, wherein: (i) the first cleavase is an S. pyogenes (Spy)Cas9 cleavase, said SpyCas9 cleavase comprising a R1333K mutation within its protospacer adjacent motif recognition domain; and (ii) the second cleavase is an N. meningitidis (Nme)Cas9 cleavase, a C. jejuni (Cje)Cas9 cleavase, or an S. muelleri (Smu) Cas9 cleavase. In some embodiments, the targeted LNP may further comprise a first guide RNA that directs the first cleavase to a first genomic locus; and a second guide RNA that directs the second cleavase to a second genomic locus, wherein the second genomic locus is different from the first genomic locus. In some embodiments, a targeted LNP may comprise the ORF encoding the fusion protein, the first guide RNA, the second guide RNA, an amine lipid, a helper lipid, a neutral lipid, a stealth lipid, and a targeting domain disclosed herein. In some embodiments, the targeted LNP is targeted to the lung, spleen, or bone marrow.

In some embodiments, the Targeted LNP comprises (a) a polynucleotide comprising an open reading frame (ORF) encoding a fusion protein, wherein the fusion protein comprises a first cleavase and a second cleavase; (b) a first guide RNA that directs the first cleavase to a first genomic locus; and (c) a second guide RNA that directs the second cleavase to a second genomic locus, wherein the second genomic locus is different from the first genomic locus. In some embodiments, a targeted LNP may comprise (a)-(c), an amine lipid, a helper lipid, a neutral lipid, a stealth lipid, and a targeting lipid component disclosed herein. In some embodiments, a targeted LNP may comprise (a)-(c), an amine lipid, a helper lipid, a neutral lipid, a stealth lipid, and a targeting domain disclosed herein. In some embodiments, the targeted LNP is targeted to the lung, spleen, or bone marrow.

In some embodiments, the targeted LNP comprises a polynucleotide comprising an open reading frame (ORF) encoding (a) a fusion protein, wherein the fusion protein comprises a first cleavase and a second cleavase, wherein: (i) the first cleavase is an S. pyogenes (Spy)Cas9 cleavase, said SpyCas9 cleavase comprising a R1333K mutation within its protospacer adjacent motif recognition domain; and (ii) the second cleavase is an N. meningitidis (Nme)Cas9 cleavase, a C. jejuni (Cje)Cas9 cleavase, or an S. muelleri (Smu) Cas9 cleavase; and (b) a first guide RNA that directs the first cleavase to a first genomic locus; and (c) a second guide RNA that directs the second cleavase to a second genomic locus, wherein the second genomic locus is different from the first genomic locus. some embodiments, a targeted LNP may comprise (a)-(c), an amine lipid, a helper lipid, a neutral lipid, a stealth lipid, and a targeting lipid component disclosed herein. In some embodiments, a targeted LNP may comprise (a)-(c), an amine lipid, a helper lipid, a neutral lipid, a stealth lipid, and a targeting domain disclosed herein. In some embodiments, the targeted LNP is targeted to the lung, spleen, or bone marrow.

In some embodiments, the targeted LNP comprises (a) a first polypeptide comprising an ORF encoding the first polypeptide, wherein the first polypeptide comprises a first cleavase and a first intein, wherein the first cleavase is an S. pyogenes (Spy)Cas9 cleavase, said SpyCas9 cleavase comprising a R1333K mutation within its protospacer adjacent motif recognition domain; (b) a second polynucleotide comprising an ORF encoding the second polypeptide, wherein the second polypeptide comprises a second cleavase and a second intein capable of binding to the first intein, wherein the second cleavase is an N. meningitidis (Nme)Cas9 cleavase, a C. jejuni (Cje)Cas9 cleavase, or a Simonsiella muelleri (Smu)Cas9 cleavase, and wherein the first polypeptide binds to the second polypeptide through intein catalysis. In some embodiments, the targeted LNP may further comprise a first guide RNA that directs the first cleavase to a first genomic locus; and a second guide RNA that directs the second cleavase to a second genomic locus, wherein the second genomic locus is different from the first genomic locus. In some embodiments, a targeted LNP may comprise the ORF encoding the fusion protein, the first guide RNA, the second guide RNA, an amine lipid, a helper lipid, a neutral lipid, a stealth lipid, and a targeting domain disclosed herein. In some embodiments, the targeted LNP is targeted to the lung, spleen, or bone marrow.

In some embodiments, the targeted LNP comprises (a) a first polynucleotide comprising an ORF encoding the first polypeptide, wherein the first polypeptide comprises first cleavase and a first intein, wherein the first cleavase is a S. pyogenes (Spy)Cas9 cleavase, said SpyCas9 cleavase comprising a R1333K mutation within its protospacer adjacent motif recognition domain; (b) a second polynucleotide comprising an ORF encoding the second polypeptide, wherein the second polypeptide comprises a second cleavase and a second intein capable of binding the first intein, wherein the second cleavase is an N. meningitidis (Nme)Cas9 cleavase, a C. jejuni (Cje) Cas9 cleavase, or a Simonsiella muelleri (Smu) Cas9 cleavase; and wherein the first polypeptide binds to the second polypeptide through intein catalysis; (c) a first guide RNA that directs the first cleavase to a first genomic locus; and (d) a second guide RNA that directs the second cleavase to a second genomic locus, wherein the second genomic locus is different from the first genomic locus. In some embodiments, a targeted LNP may comprise (a)-(c), an amine lipid, a helper lipid, a neutral lipid, a stealth lipid, and a targeting lipid component. In some embodiments, a targeted LNP may comprise (a)-(d), an amine lipid, a helper lipid, a neutral lipid, a stealth lipid, and a targeting domain. In some embodiments, the targeted LNP is targeted to the lung, spleen, or bone marrow.

In some embodiments, the targeted LNP comprises a nucleic acid, e.g., an RNA, component that includes one or more of an RNA-guided DNA binding agent, a Cas nuclease mRNA, a Class 2 Cas nuclease mRNA, a Cas9 mRNA, and a gRNA. In some embodiments, a targeted LNP may include a Class 2 Cas nuclease and a gRNA as the RNA component. In some embodiments, a targeted LNP may comprise the RNA component, an amine lipid, a helper lipid, a neutral lipid, a stealth lipid, and a targeting lipid component. In some embodiments, a targeted LNP may comprise the RNA component, an amine lipid, a helper lipid, a neutral lipid, a stealth lipid, and a targeting domain.

In some embodiments, a targeted LNP comprises an RNA component, which may comprise an mRNA, such as an mRNA encoding a Cas nuclease. In one embodiment, the RNA component may comprise a Cas9 mRNA. In some compositions comprising an mRNA encoding a Cas nuclease, the lipid nucleic acid assembly further comprises a gRNA nucleic acid, such as a gRNA. In some embodiments, the RNA component comprises a Cas nuclease mRNA and a gRNA. In some embodiments, the RNA component comprises a Class 2 Cas nuclease mRNA and a gRNA.

In some embodiments, a targeted LNP may comprise an mRNA encoding a Cas nuclease an amine lipid, a helper lipid, a neutral lipid, and a PEG lipid. In certain LNPs comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, the PEG lipid is PEG2k-DMG or PEG2k-C11. In specific compositions comprising an mRNA encoding a Cas nuclease such as a Class 2 Cas nuclease, the amine lipid is selected from Lipid A and its equivalents, such as an acetal analog of Lipid A, or amine lipids provided in WO2020/219876; or Lipid D or amine lipids provided in WO2020/072605.

In some embodiments, a targeted LNP may comprise a gRNA. In some embodiments, a targeted LNP may comprise an amine lipid, a gRNA, a helper lipid, a neutral lipid, and a PEG lipid. In certain LNPs comprising a gRNA, the helper lipid is cholesterol. In some compositions comprising a gRNA, the neutral lipid is DSPC. In additional embodiments comprising a gRNA, the PEG lipid is PEG2k-DMG or PEG2k-C11. In some embodiments, the amine lipid is selected from Lipid A and its equivalents, such as an acetal analog of Lipid A, or amine lipids provided in WO2020/219876 and their equivalents; or Lipid D or amine lipids provided in WO2020/072605 and their equivalents.

In one embodiment, a targeted LNP may comprise an sgRNA. In one embodiment, a targeted LNP may comprise a Cas9 sgRNA. In one embodiment, a targeted LNP may comprise a Cpf1 sgRNA. In some compositions comprising an sgRNA, the lipid nucleic acid assembly includes an amine lipid, a helper lipid, a neutral lipid, and a PEG lipid. In certain compositions comprising an sgRNA, the helper lipid is cholesterol. In other compositions comprising an sgRNA, the neutral lipid is DSPC. In additional embodiments comprising an sgRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D or amine lipids provided in WO2020/072605.

In some embodiments, a targeted LNP comprises an mRNA encoding a Cas nuclease and a gRNA, which may be an sgRNA. In one embodiment, a targeted LNP may comprise an amine lipid, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D or amine lipids provided in WO2020/072605.

In some embodiments, the targeted LNPs include a Cas nuclease mRNA, such as a Class 2 Cas mRNA and at least one gRNA. In some embodiments, the targeted LNP includes a ratio of gRNA to Cas nuclease mRNA, such as Class 2 Cas nuclease mRNA from about 25:1 to about 1:25 wt/wt. In some embodiments, the lipid nucleic acid assembly formulation includes a ratio of gRNA to Cas nuclease mRNA, such as Class 2 Cas nuclease mRNA from about 10:1 to about 1:10. In some embodiments, the lipid nucleic acid assembly formulation includes a ratio of gRNA to Cas nuclease mRNA, such as Class 2 Cas nuclease mRNA from about 8:1 to about 1:8. As measured herein, the ratios are by weight. In some embodiments, the lipid nucleic acid assembly formulation includes a ratio of gRNA to Cas nuclease mRNA, such as Class 2 Cas mRNA from about 5:1 to about 1:5. In some embodiments, ratio range is about 3:1 to about 1:3, about 2:1 to about 1:2, about 5:1 to about 1:2, about 5:1 to about 1:1, about 3:1 to about 1:2, about 3:1 to about 1:1, about 3:1, about 2:1 to about 1:1. In some embodiments, the gRNA to mRNA ratio is about 3:1 or about 2:1. In some embodiments the ratio of gRNA to Cas nuclease mRNA, such as Class 2 Cas nuclease is about 1:1. In some embodiments the ratio of gRNA to Cas nuclease mRNA, such as Class 2 Cas nuclease is about 1:2. The ratio may be about 25:1, 10:1, 5:1, 3:1, 2:1, 1:1, 1:2, 1:3, 1:5, 1:10, or 1:25.

The targeted LNPs disclosed herein may include a template nucleic acid. The template nucleic acid may be co-formulated with an mRNA encoding a Cas nuclease, such as a Class 2 Cas nuclease mRNA. In some embodiments, the template nucleic acid may be co-formulated with a guide RNA. In some embodiments, the template nucleic acid may be co-formulated with both an mRNA encoding a Cas nuclease and a guide RNA. In some embodiments, the template nucleic acid may be formulated separately from an mRNA encoding a Cas nuclease or a guide RNA. The template nucleic acid may be delivered with, or separately from the LNPs. In some embodiments, the template nucleic acid may be single- or double-stranded, depending on the desired repair mechanism. The template may have regions of homology to the target DNA, or to sequences adjacent to the target DNA.

In some embodiments, the targeted LNPs disclosed herein may be administered by intravenous, intradermal, subcutaneous, inhalation, intranasal, or intramuscular delivery.

E. Contacting Cells with LNP

In some embodiments, the LNP is pretreated with a serum factor before contacting the cell. In some embodiments, the LNP is pretreated with a primate serum factor before contacting the cell. In some embodiments, the LNP is pretreated with a human serum factor before contacting the cell.

In some embodiments, the methods disclosed herein comprise preincubating a serum factor and the LNP for about 30 seconds to overnight. In some embodiments, the preincubation step comprises preincubating a serum factor and the LNP for about 1 minute to 1 hour. In some embodiments, it comprises preincubating for about 1-30 minutes.

In some embodiments, the LNP compositions are administered sequentially. In some embodiments, the LNP compositions are administered simultaneously. In some embodiments, the population of cells is contacted with 3 LNP compositions. In some embodiments, the population of cells is contacted with 4 LNP compositions.

In some embodiments, the cells are frozen between sequential contacting or editing steps.

In some embodiments, the LNP is pretreated with a serum factor before contacting the cell. In some embodiments, the LNP is pretreated with a human serum before contacting the cell. In some embodiments, the LNP is pretreated with a serum replacement, e.g., a commercially available serum replacement, preferably wherein the serum replacement is appropriate for ex vivo use.

In some embodiments, a LNP is provided to a “non-activated” cell. A “non-activated” cell refers to a cell that has not been stimulated in vitro. In some embodiments, a “non-activated” T cell may have been stimulated in vivo (e.g., by antigen) while in the body, however said cell may be referred to as non-activated herein if said cell has not been stimulated in vitro in culture. An “activated” cell is also useful in the methods disclosed herein and can refer to a cell that has been stimulated in vitro. Agents for activating cells in vitro are provided herein and are known in the art, particularly for activation of T cells or B cells.

In some embodiments, the T cell is activated prior to contact with a LNP, is activated in between contact with LNPs, or is activated after contact with a LNP.

While the invention is described in conjunction with the illustrated embodiments, it is understood that they are not intended to limit the invention to those embodiments. On the contrary, the invention is intended to cover all alternatives, modifications, and equivalents, including equivalents of specific features, which may be included within the invention as defined by the appended claims.

### VII. Exemplary DNA Molecules, Vectors, Expression Constructs, Host Cells, and Production Methods

In certain embodiments, the disclosure provides one or more DNA molecule comprising a sequence encoding any of the nucleic acids (e.g., mRNAs) encoding a cleavase or a fusion protein described herein. In some embodiments, the DNA molecule may further include, but is not limited to, promoters, enhancers, and regulatory sequences.

In certain embodiments, the disclosure provides one or more DNA molecule comprising a sequence encoding one or more guide RNA as described herein. In some embodiments, the DNA molecule further may further include, but is not limited to, promoters, enhancers, and regulatory sequences. In some embodiments, the DNA molecule further comprises a promoter operably linked to the sequence encoding a guide RNA described herein.

In some embodiments, the DNA molecule further comprises a promoter operably linked to the sequence encoding any of the nucleic acids (e.g., mRNAs) encoding the cleavase or the fusion protein or the guide RNAs described herein. In some embodiments, the DNA molecule is an expression construct suitable for expression in a mammalian cell, e.g., a human cell or a mouse cell, such as a human hepatocyte or a rodent (e.g., mouse) hepatocyte. In some embodiments, the DNA molecule is an expression construct suitable for expression in a cell of a mammalian organ, e.g., a human liver or a rodent (e.g., mouse) liver. In some embodiments, the DNA molecule is a plasmid or an episome. In some embodiments, the DNA molecule is contained in a host cell, such as a bacterium or a cultured eukaryotic cell. Exemplary bacteria include proteobacteria such as E. coli. Exemplary cultured eukaryotic cells include primary hepatocytes, including hepatocytes of rodent (e.g., mouse) or human origin; hepatocyte cell lines, including hepatocytes of rodent (e.g., mouse) or human origin; human cell lines; rodent (e.g., mouse) cell lines; Chinese hamster ovary (CHO) cells; microbial fungi, such as fission or budding yeasts, e.g., Saccharomyces, such as S. cerevisiae; and insect cells.

In some embodiments, a method of producing a nucleic acid (e.g., an mRNA or guide RNA) disclosed herein is provided. In some embodiments, such a method comprises contacting a DNA molecule described herein with an RNA polymerase under conditions permissive for transcription. In some embodiments, the contacting is performed in vitro, e.g., in a cell-free system. In some embodiments, the RNA polymerase is an RNA polymerase of bacteriophage origin, such as T7 RNA polymerase. In some embodiments, NTPs are provided that include at least one modified nucleotide as discussed above. In some embodiments, the NTPs include at least one modified nucleotide as discussed above and do not comprise UTP.

In some embodiments, a nucleic acid (e.g., an mRNA or guide RNA) disclosed herein alone or together with one or more guide RNAs, may be comprised within or delivered by a vector system of one or more vectors. In some embodiments, one or more of the vectors, or all of the vectors, may be DNA vectors. In some embodiments, one or more of the vectors, or all of the vectors, may be RNA vectors. In some embodiments, one or more of the vectors, or all of the vectors, may be circular. In other embodiments, one or more of the vectors, or all of the vectors, may be linear. In some embodiments, one or more of the vectors, or all of the vectors, may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Non-limiting exemplary viral vectors include adeno-associated virus (AAV) vectors, lentivirus vectors, adenovirus vectors, helper dependent adenoviral (HDAd) vectors, herpes simplex virus (HSV-1) vectors, bacteriophage T4, baculovirus vectors, and retrovirus vectors. In some embodiments, the viral vector may be an AAV vector. In other embodiments, the viral vector may be a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas protein, while a second AAV vector may contain one or more guide sequences.

In some embodiments, the vector may be capable of driving expression of one or more coding sequences, such as the coding sequence of an mRNA disclosed herein, in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoter may be constitutive, inducible, or tissue-specific. In some embodiments, the promoter may be a constitutive promoter. Non-limiting exemplary constitutive promoters include cytomegalovirus (CMV) immediate early promoter, simian virus (SV40) promoter, adenovirus major late promoter (MLP), Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding at least one guide RNA. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a ribonucleoprotein complex with the cleavase or the fusion protein disclosed herein. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA. In other embodiments, the crRNA and the trRNA may be driven by their corresponding promoters on the same vector. In yet other embodiments, the crRNA and the trRNA may be encoded by different vectors.

In some embodiments, the composition may comprise a vector system, wherein the system comprises more than one vector. In some embodiments, the vector system may comprise one single vector. In other embodiments, the vector system may comprise two vectors. In additional embodiments, the vector system may comprise three vectors. When multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

### VIII. Cells

In some embodiments, a cell contacted with the fusion protein disclosed herein is a human cell.

In some embodiments, a cell is contacted with a fusion protein disclosed herein comprising (a) a first cleavase and a guide RNA (gRNA) that targets at least one genomic locus and that is cognate to the first cleavase; and (b) a second cleavase and a gRNA that targets at least one genomic locus and that is cognate to the second cleavase, wherein the first cleavase is orthogonal to the second cleavase, thereby generating a deletion in the cell.

In some embodiments, a cell is contacted with a fusion protein disclosed herein comprising (a) a first cleavase and a guide RNA (gRNA) that targets at least one genomic locus and that is cognate to the first cleavase; and (b) a second cleavase and a gRNA that targets at least one genomic locus and that is cognate to the second cleavase, wherein the first cleavase is orthogonal to the second cleavase, thereby excising a DNA sequence between a first cleavage site cleaved by the first cleavase and a second cleavage site cleaved by the second cleavase; in some embodiments, the cell is cultured, thereby producing a population of cells comprising edited cells comprising deletions.

In some embodiments, a cell is treated in vitro with any method or composition disclosed herein. In some embodiments, a cell is treated in vivo with any method or composition disclosed herein.

In some embodiments, the cell in any of the embodiments provided herein is engineered by a fusion protein disclosed herein.

In some embodiments, the fusion protein disclosed herein is delivered to the cell via electroporation. In some embodiments, the fusion protein disclosed herein is delivered to the cell via at least one lipid nanoparticle (LNP). In some embodiments, the fusion protein disclosed herein is delivered to the cell on at least one vector. In some embodiments, the fusion protein disclosed herein is delivered as at least one nucleic acid encoding the fusion protein disclosed herein. In some embodiments, the at least one nucleic acid comprises at least one mRNA. In some embodiments, the fusion protein disclosed herein is delivered to the cell as at least one polypeptide or at least one mRNA. In some embodiments, the at least one gRNA is delivered to the cell as at least one polynucleotide that encodes the gRNA.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”).

In some embodiments, the cell is derived from a progenitor cell before editing. In some embodiments, the cell is an induced pluripotent stem cell (iPSC).

Cells used in ACT therapy are included, such as mesenchymal stem cells (e.g., isolated from bone marrow (BM), peripheral blood (PB), placenta, umbilical cord (UC) or adipose); hematopoietic stem cells (HSCs; e.g. isolated from BM); mononuclear cells (e.g., isolated from BM or PB); endothelial progenitor cells (EPCs; isolated from BM, PB, and UC); neural stem cells (NSCs); limbal stem cells (LSCs); or tissue-specific primary cells or cells derived therefrom (TSCs). Cells used in ACT therapy further include induced pluripotent stem cells (iPSCs; see e.g., Mahla, International J. Cell Biol. 2016 (Article ID 6940283): 1-24 (2016)) that may be induced to differentiate into other cell types including e.g., islet cells, neurons, and blood cells; ocular stem cells; pluripotent stem cells (PSCs); embryonic stem cells (ESCs); cells for organ or tissue transplantations such as islet cells, cardiomyocytes, thyroid cells, thymocytes, neuronal cells, skin cells, retinal cells, chondrocytes, myocytes, and keratinocytes.

In some embodiments, the cell is a human cell, such as a cell from a subject. In some embodiments, the cell is isolated from a human subject. In some embodiments, the cell is isolated from a patient. In some embodiments, the cell is isolated from a donor. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods disclosed herein are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is maintained in culture. In some embodiments, the cell is transplanted into a patient. In some embodiments, the cell is removed from a subject, genetically modified ex vivo, and then administered back to the same patient. In some embodiments, the cell is removed from a subject, genetically modified ex vivo, and then administered to a subject other than the subject from which it was removed.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, a population of cells comprises any cell edited using any method or composition disclosed herein.

In some embodiments, a population of cells comprises edited T cells, and wherein at least 30%, 40%, 50%, 55%, 60%, 65% of the cells of the population have a memory phenotype (CD27+, CD45RA+).

In some embodiments, a population of cells comprises non-activated immune cells. In some embodiments, the population of cells comprises activated immune cells.

In some embodiments, a population of cells comprises T cells and is responsive to repeat stimulation after editing. In some embodiments, the population of cells is cultured, expanded, differentiated, or proliferated ex vivo.

Both the foregoing general description and detailed description, as well as the following examples, are exemplary and explanatory only and are not restrictive of the teachings.

The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### EXAMPLES

The following examples are provided to illustrate certain disclosed embodiments and are not to be construed as limiting the scope of this disclosure in any way.

### Example 1. Materials and Methods

In Vitro Transcription (“IVT”) of Nuclease mRNA

Capped and polyadenylated mRNA containing N1-methyl pseudo-U was generated by in vitro transcription using a linearized plasmid DNA template and T7 RNA polymerase. The linearized plasmid DNA containing a T7 promoter, and a sequence for transcription was linearized by restriction endonuclease digestion followed by heat inactivation of the reaction mixture, and purified from enzyme and buffer salts. Following the IVT reaction, the modified mRNA was synthesized and purified by standard techniques.

Streptococcus pyogenes (“Spy”) Cas9 mRNA was generated from plasmid DNA encoding an open reading frame according to SEQ ID Nos: 18, 36, 94, or 110 (see sequences in Table 37). When the sequences cited in this paragraph are referred to below with respect to RNAs, it is understood that Ts should be replaced with Us (which can be modified nucleosides as described above). Messenger RNAs used in the Examples. Guide RNAs were chemically synthesized by commercial vendors or using standard in vitro synthesis techniques with modified nucleotides.

### LNP Formulation

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, distearoylphosphatidylcholine (DSPC), and 1,2-dimyristoyl-rac-glycero-3-methoxypolyethylene glycol-2000 (PEG2K-DMG) (e.g., catalog #GM-020 from NOF, Tokyo, Japan) in a molar ratio of 50% Lipid A, 38% cholesterol, 9% DSPC, and 3% PEG2k-DMG. The LNPs were formulated with a lipid amine to RNA phosphate (N:P) molar ratio of about 6.

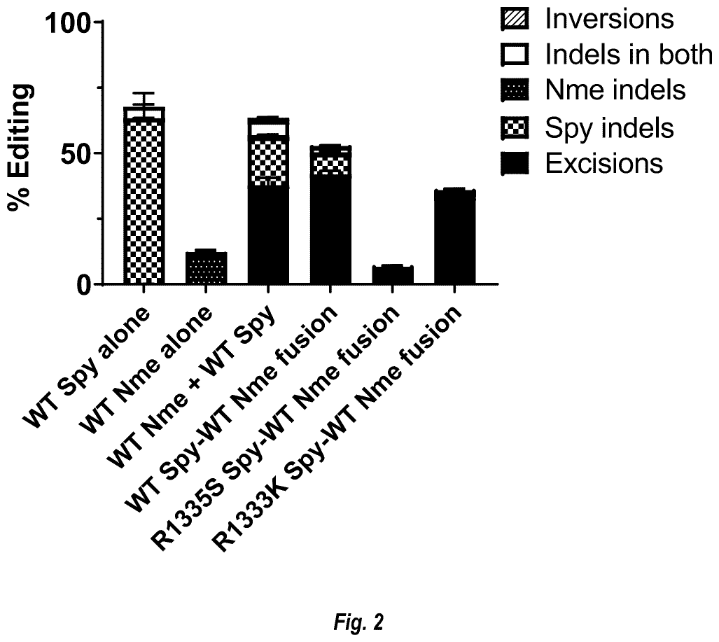

The LNPs were prepared using a cross-flow technique utilizing impinging jet mixing of the lipid in ethanol with two volumes of RNA solutions and one volume of water. First, the lipid in ethanol was mixed through a mixing cross with the two volumes of RNA solution. Then, a fourth stream of water was mixed with the outlet stream of the cross through an inline tee (See WO2016010840 FIG. 2). The LNPs were held for 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 m sterile filter. The final LNPs were characterized to determine the encapsulation efficiency, polydispersity index, and average particle size. The final LNPs were stored at 4° C. or −80° C. until further use.

sgRNA and Cas9 mRNA Lipofection

Lipofection of Cas9 mRNA and gRNAs used pre-mixed lipid formulations. The lipofection reagent contained ionizable Lipid A, cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA (e.g., Cas9 mRNA and gRNA) at a lipid amine to RNA phosphate (N:P) molar ratio of about 6 to produce lipid nucleic acid mixtures. An mRNA comprising a Cas9 ORF of Table 37 was produced by in vitro transcription (IVT) as described in WO2019/067910, see e.g., ¶354, using a 2 hour IVT reaction time and purifying the mRNA by LiCl precipitation followed by tangential flow filtration.

Lipofections were performed with a ratio of gRNA to mRNA of 1:2 by weight, unless otherwise indicated. Briefly, cells were incubated at 37° C., 5% CO2 for 24 hours prior to treatment with the lipid nucleic acid mixtures. Lipid nucleic acid mixtures were incubated in media containing 6% cynomolgus monkey or 6% fetal bovine serum (FBS) at 37° C. for 10 minutes. Post-incubation, the lipid nucleic acid mixtures were added to the cells (e.g., primary mouse hepatocytes) in an 8 or 12 point 3-fold dose response curve starting at 300 ng Cas9 mRNA, unless otherwise indicated. The cells were lysed 72 hours post-treatment, unless otherwise indicated, for NGS analysis as described in Example 1.

### Next-Generation Sequencing (“NGS”) and Analysis for Editing Efficiency

DNA was extracted using a commercial kit according to the manufacturer's protocol, for example QuickExtract™ DNA Extraction Solution (Lucigen, Cat. QE09050). To quantitatively determine the efficiency of editing at the target location in the genome, deep sequencing was utilized to identify the presence of excisions, insertions, deletions, and inversions introduced by gene editing. PCR primers were designed within the gene of interest (e.g., TTR). When the gene of interest was a SEAP reporter, a second set of PCR primers were designed around the target site within the gene of interest and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., mm10) after eliminating those having low quality scores. Reads that overlapped the target region of interest were re-aligned to the local genome sequence to improve the alignment. Then the number of wild-type reads versus the number of reads which contain excisions, inversions or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. Five editing percentages were calculated when using orthogonal Cas9-Cas9 fusion nucleases, including:
1) Spy indel percentage, resulting from insertions and/or deletions effected by the SpyCas9 moiety of orthogonal Cas9-Cas9 fusion, guided by a SpyCas9 sgRNA to its target cut site,
2) Nme indel percentage, resulting from insertions and/or deletions effected by the Nme2Cas9 moiety of orthogonal Cas9-Cas9 fusion, guided by a Nme2Cas9 sgRNA to its target cut site,
3) Excision percentage, resulting from both the SpyCas9 and Nme2Cas9 moieties of orthogonal Cas9-Cas9 fusion effecting cuts at both cut sites targeted by a SpyCas9 and a Nme2Cas9, causing an excision of the sequence comprised between both cut sites,
4) “Indels in both” percentage, resulting from insertions and/or deletions effected by both the SpyCas9 and Nme2Cas9 moieties of orthogonal Cas9-Cas9 fusion at both cut sites targeted by the Spy and Nme sgRNAs, without excision of the sequence between the cut sites,
5) Inversion percentage, resulting from both the SpyCas9 and Nme2Cas9 moieties of orthogonal Cas9-Cas9 fusion effecting cuts at both cut sites targeted by a SpyCas9 and a Nme2Cas9, causing an inversion of the sequence comprised between both cut sites.

Each of the five editing type percentages is defined as the total number of sequencing reads with each edit divided by the total number of reads overlapping both predicted cut sites.

Reported measurements in the Pipeline Columns used for excision analysis were obtained as follows. Amplicon-Seq libraries were sequenced with pair-end 150 bp. Two reads in a pair were merged into a single read with the following parameters: -m 5 -M 150. Merged reads shorter than 150 bp were discarded. The rest of the reads were aligned to the reference genome. Unmerged R1 and R2 reads were aligned to the reference genome separately. Their coverage on the target site was calculated. Alignments from the unmerged end with higher coverage on the target site were combined with alignments of merged reads. Alignments more than 150 bp away from the gRNA target site were discarded. Reads with PHRED score below 10 in any position were also discarded. Reads with alignment near the target site were re-aligned to the local genomic sequence with Smith-Waterman algorithm (Smith & Waterman, 1981; Zhao et al., 2013). Those reads were simultaneously aligned to a predicted inversion sequence with the same algorithm and parameters. The reads were classified as “inversion” if the alignment score with the predicted inversion was higher than with the wild-type reference sequence. Nucleotides within 5 base pairs from the target Cas9 cut sites (i.e., within an “indel window”) were evaluated for insertions or deletions (collectively “indels”). Alignments that failed to completely cover both indel windows of the two guides were discarded. An “excision” is defined as a single long deletion that starts within the indel window of one guide RNA and ends within the indel window of the other guide RNA. If indels were detected in both windows but failed to satisfy the excision criteria, the reads were classified as “non excision indels”. Conversely, if indels were detected in only one of the two windows, the reads were then classified as “indel only in left” or “indel only in right” (e.g., “single guide indel”, “Spy only indel”, “Nme only indel”), with left defined as the guide with lower coordinate target site.

### Example 2—Wild-Type Double-Cleavase Editing and Excision Activity

A SpyCas9 cleavase tethered to NmeCas9 cleavase was tested in HEK-Blue IL-1B SEAP reporter cells for its editing and excision activity. HEK-Blue IL-1B cells (Invivogen, Cat #hkb-il1b) were grown in Growth Medium (DMEM, 4.5 g/l glucose, 2 mM L-Glutamine, 10% (v/v) fetal bovine serum (FBS), 50 U/ml penicillin, 50 mg/ml streptomycin, 100 mg/ml Normocin™ (Invivogen, Cat #ant-nr-1)) or Test Medium (DMEM, 4.5 g/l glucose, 2 mM L-Glutamine, 10% (v/v) heat-inactivated FBS (30 min at 56° C.), 50 U/ml penicillin, 50 mg/ml streptomycin, 100 mg/ml Normocin™, Hygromycin B Gold (Invivogen, Cat #ant-hg-1), Zeomicin (Invivogen Cat #ant-zn-05)) as per the manufacturer's instructions in 96-well plates at a density of 10,000 cells/well. Cells were incubated at 37° C. and under 5% CO₂ atmosphere for 18-20 hours and were co-transfected using Lipofectamine™ 3000 Transfection Reagent (Thermo Fisher Scientific, Cat #L3000015) as per the manufacturer's instructions (0.3 uL Lipofectamine3000/well) with 50 ng of plasmid encoding a nuclease construct, and 50 ng of plasmid encoding a Spy guide RNA targeting SEAP and/or 50 ng of plasmid encoding a Nme guide RNA targeting SEAP. The targeting sequences of Spy and or Nme guides targeting SEAP are shown in Table 5. Three nuclease plasmids were used, each encoding one of: a wild-type NmeCas9 (SEQ ID NO: 106), a wild-type SpyCas9 (SEQ ID NO: 110), or a WT SpyCas9-WT NmeCas9 fusion construct (SEQ ID NO: 96).

TABLE 5
SEAP Guide RNA target sequences

| | | SEQ ID |
|---|---|---|
| | | NO of |
| | | Target |
| Guide ID | Target Sequence | sequence. |
| plCR012468 | UGGCGCUUCGCCAGCCAUUCCUGC | 261 |
| plCR012469 | CCCGGGGCGGUCCCGGUCCAAGCG | 262 |
| plG000665 | CUCCCUGAUGGAGAUGACAG | 263 |
| plG012746 | CACCUUGGCUGUAGUCAUCU | 264 |
| plG012684 | CAGUGCGGUUCCACACAUAC | 265 |
| plG012685 | AAGCCUGCAUGAGCUCAGUG | 266 |
| plG012686 | AUGGGUCACAGACGGGUCCA | 267 |
| plG012687 | CUCAAAGAGACCCAUGAGAU | 268 |
| plG012688 | AUCUCGUAUUUCAUGUCUCC | 269 |

Three days after transfection, media was removed and the cells were lysed with QuickExtract™ DNA Extraction Solution (Lucigen, Cat. QE09050). NGS analysis was performed as described in Example 1. Percent editing in HEK-Blue cells is shown in Table 6, and illustrated in FIG. 1.

TABLE 6
Editing as a percentage of total NGS reads in HEK-Blue cells

| | | | Nme9 only | | Spy9 only | | | | Indels | | | |
|---|---|---|---|---|---|---|---|---|---|---|---|---|
| | Guide ID | | indels | | indels | | Excisions | | in both | | Inversions | |
| Nuclease | Spy | Nme | Mean | SD | Mean | SD | Mean | SD | Mean | SD | Mean | SD |
| WT SpyCas9 (n = 1) | plG000665 | None | 0.00 | N/A | 0.00 | N/A | 0.00 | N/A | 0.00 | N/A | 0.00 | N/A |
| | plG012688 | | 0.00 | N/A | 10.90 | N/A | 0.00 | N/A | 0.00 | N/A | 0.00 | N/A |
| | plG012687 | | 0.00 | N/A | 14.88 | N/A | 0.00 | N/A | 14.68 | N/A | 0.00 | N/A |
| | plG012686 | | 0.02 | N/A | 19.57 | N/A | 0.12 | N/A | 7.51 | N/A | 0.00 | N/A |
| | plG012685 | | 0.03 | N/A | 30.28 | N/A | 0.00 | N/A | 3.47 | N/A | 0.00 | N/A |
| | plG012684 | | 0.02 | N/A | 16.08 | N/A | 0.18 | N/A | 0.29 | N/A | 0.00 | N/A |
| | plG012746 | | 0.00 | N/A | 8.75 | N/A | 0.00 | N/A | 2.72 | N/A | 0.00 | N/A |
| WT NmeCas9 (n = 1) | None | plCR012468 | 2.34 | N/A | 0.00 | N/A | 0.00 | N/A | 0.00 | N/A | 0.00 | N/A |
| SpyCas9-NmeCas9 | None | plCR012468 | 0.19 | N/A | 0.00 | N/A | 0.00 | N/A | 0.00 | N/A | 0.00 | N/A |
| fusion (n = 1) | | | | | | | | | | | | |
| SpyCas9-NmeCas9 | plG000665 | None | 0.00 | 0.00 | 0.00 | 0.00 | 0.00 | 0.00 | 0.00 | 0.00 | 0.00 | 0.00 |
| fusion (n = 2) | plG012688 | | 0.00 | 0.00 | 2.53 | 0.19 | 0.00 | 0.00 | 0.01 | 0.02 | 0.00 | 0.00 |
| | plG012687 | | 0.00 | 0.00 | 3.55 | 0.06 | 0.00 | 0.00 | 0.01 | 0.02 | 0.00 | 0.00 |
| | plG012686 | | 0.00 | 0.00 | 3.31 | 0.47 | 0.00 | 0.00 | 0.00 | 0.00 | 0.00 | 0.00 |
| | plG012685 | | 0.01 | 0.01 | 4.93 | 1.67 | 0.00 | 0.00 | 0.00 | 0.00 | 0.00 | 0.00 |
| | plG012684 | | 0.01 | 0.02 | 4.54 | 0.10 | 0.00 | 0.00 | 0.00 | 0.00 | 0.00 | 0.00 |
| | plG012746 | | 0.01 | 0.01 | 1.50 | 0.26 | 0.00 | 0.00 | 0.00 | 0.00 | 0.00 | 0.00 |
| SpyCas9-NmeCas9 | plG000665 | plCR012468 | 0.15 | 0.04 | 0.02 | 0.00 | 0.00 | 0.00 | 0.00 | 0.00 | 0.00 | 0.00 |
| fusion (n = 2) | plG012688 | | 0.10 | 0.08 | 1.20 | 0.23 | 26.77 | 1.65 | 0.21 | 0.25 | 0.04 | 0.01 |
| | plG012687 | | 0.11 | 0.03 | 1.63 | 0.16 | 21.23 | 0.13 | 1.22 | 1.19 | 0.01 | 0.02 |
| | plG012686 | | 0.08 | 0.03 | 1.93 | 0.91 | 14.28 | 0.91 | 1.23 | 1.32 | 0.04 | 0.06 |
| | plG012685 | | 0.03 | 0.04 | 2.03 | 0.31 | 13.16 | 1.36 | 0.39 | 0.47 | 0.05 | 0.01 |
| | plG012684 | | 0.03 | 0.02 | 3.63 | 0.42 | 2.84 | 0.35 | 0.15 | 0.01 | 0.05 | 0.07 |
| | plG012746 | | 0.06 | 0.02 | 0.36 | 0.04 | 14.99 | 0.80 | 0.01 | 0.02 | 0.03 | 0.04 |

### Example 3—PAM-Attenuated Cleavase Fusion Editing and Excision Activity

A PAM-attenuated SpyCas9 tethered to NmeCas9 (orthogonal Cas9-Cas9 fusion construct) was tested in HEK-Blue IL-1B SEAP reporter cells for its editing and excision activity. HEK-Blue IL-1B cells (Invivogen, Cat #hkb-il1b) were grown in Growth Medium (DMEM, 4.5 g/l glucose, 2 mM L-Glutamine, 10% (v/v) fetal bovine serum (FBS), 50 U/ml penicillin, 50 mg/ml streptomycin, 100 mg/ml Normocin™ (Invivogen, Cat #ant-nr-1)) or Test Medium (DMEM, 4.5 g/l glucose, 2 mM L-Glutamine, 10% (v/v) heat-inactivated FBS (30 min at 56° C.), 50 U/ml penicillin, 50 mg/ml streptomycin, 100 mg/ml Normocin™, Hygromycin B Gold (Invivogen, Cat #ant-hg-1), Zeomicin (Invivogen Cat #ant-zn-05)) as per the manufacturer's instructions in 96-well plates at a density of 10,000 cells/well. Cells were incubated at 37° C. and under 5% CO₂ atmosphere for 18-20 hours and were co-transfected using Lipofectamine™ 3000 Transfection Reagent (Thermo Fisher Scientific, Cat #L3000015) as per manufacturer's instructions (0.3 uL Lipofectamine3000/well) with 50 ng of plasmid encoding a nuclease construct, and 50 ng of plasmid encoding a Spy guide RNA (SEQ ID NO: 257) targeting SEAP and/or 50 ng of plasmid encoding a Nme guide RNA (SEQ ID NO: 238) targeting SEAP. Five nuclease plasmids were used, each encoding one of: a wild-type NmeCas9 (SEQ ID NO: 106), a wild-type SpyCas9 (SEQ ID NO: 110), an R1335S SpyCas9 tethered to a wild-type NmeCas9 (SEQ ID NO: 98), an R1333K SpyCas9 tethered to a wild-type NmeCas9 (SEQ ID NO: 100), and a wild-type SpyCas9 tethered to a wild-type NmeCas9 (SEQ ID NOs: 102). The orthogonal Cas9-Cas9 fusion constructs are described in Table 7.

Three days after transfection, media was removed and the cells were lysed with QuickExtract™ DNA Extraction Solution (Lucigen, Cat. QE09050). NGS analysis was performed as described in Example 1. Percent editing in HEK-Blue cells is shown in Table 8, and illustrated in FIG. 2.

TABLE 7
Nuclease sequences

| | SEQ | SpyCas9 | | NmeCas9 | Relative orientation |
|---|---|---|---|---|---|
| Construct | ID NO. | moiety | Linker | moiety | of nuclease moieties |
| WT Spy-WT Nme | 102 | WT Spy | linker | WT Nme | WT Spy-linker 81aa- |
| fusion | | | 81aa | | WT Nme |
| R1335S Spy-WT | 98 | R1335S | linker | WT Nme | R1335S Spy-linker |
| Nme fusion | | Spy | 81aa | | 81aa-WT Nme |
| R1333K Spy-WT | 100 | R1333K | linker | WT Nme | R1333K Spy-linker |
| Nme fusion | | Spy | 81aa | | 81aa-WT Nme |

TABLE 8
Editing as a percentage of total reads in HEK-Blue cells

| | Nme indels | | Spy indels | | Excisions | | Indels in both | | Inversions | |
|---|---|---|---|---|---|---|---|---|---|---|
| Plasmid | Mean | SD | Mean | SD | Mean | SD | Mean | SD | Mean | SD |
| WT Spy-WT Nme fusion | 0.49 | 0.14 | 8.02 | 0.96 | 41.86 | 1.39 | 2.06 | 0.39 | 0.32 | 0.27 |
| R1335S Spy-WT Nme | 2.36 | 0.52 | 0.02 | 0.03 | 4.28 | 1.04 | 0.04 | 0.07 | 0.00 | 0.00 |
| fusion | | | | | | | | | | |
| R1333K Spy-WT Nme | 0.47 | 0.08 | 1.31 | 0.07 | 33.35 | 3.19 | 0.57 | 0.23 | 0.30 | 0.11 |
| fusion | | | | | | | | | | |
| WT Nme + WT Spy | 0.75 | 0.27 | 18.31 | 0.67 | 37.79 | 2.80 | 5.48 | 0.89 | 1.22 | 0.27 |
| WT Spy alone | 0.12 | 0.10 | 63.19 | 5.36 | 0.05 | 0.05 | 4.39 | 5.26 | 0.00 | 0.00 |
| WT Nme alone | 11.69 | 0.49 | 0.00 | 0.00 | 0.00 | 0.00 | 0.62 | 0.82 | 0.00 | 0.00 |

### Example 4—Orthogonal Cas9-Cas9 Fusion Linker and Rec Domain-Null mRNA Editing in Hepa1-6 Mouse Cells and Primary Mouse Hepatocytes

Orthogonal Cas9-Cas9 fusion constructs with various amino acid linkers, as described in Table 9, were tested for excision efficacy in Hepa1-6 cells and Primary Mouse Hepatocytes (PMH). Hepa1-6 cells were cultured in DMEM media supplemented with 10% fetal bovine serum at a density of 9,000 cells/well in a 96-well plate and left to adhere overnight two days prior to transfection. PMH (Gibco, MC839) were thawed and resuspended in 50 mL Cryopreserved Hepatocyte Recovery Media (CHRM) (Invitrogen, CM7000) followed by centrifugation; cells were then resuspended in hepatocyte medium with plating supplements: William's E Medium Plating Supplements with FBS content (Gibco, Cat. A13450), pelleted by centrifugation, resuspended in media and plated at a density of 15,000 cells/well on Bio-coat collagen I coated 96-well plates (Corning #354407). Plated PMH were allowed to settle and adhere for 4-6 hours in a tissue culture incubator at 37° C. and 5% CO₂ atmosphere. After incubation PMH were checked for monolayer formation, existing media was aspirated and cells were plated with 100 uL hepatocyte maintenance medium: William's E Medium (Gibco, Cat. A12176-01) plus supplement pack (Gibco, Cat. CM3000) and left to adhere overnight prior to transfection. Both cell types were transfected using 0.6 uL/well Lipofectamine Messenger Max (ThermoFisher, Cat. LMRNA001) diluted in OptiMem Medium (ThermoFisher, Cat. 31985062) according to the manufacturer's protocol with, in each well, 100 ng SpyCas9 mRNA equivalents of a nuclease or orthogonal Cas9-Cas9 fusion construct as described in Tables 9 and 10, 50 nM SpyCas9 guide and 50 nM NmeCas9 guide. mRNA and guide were added in two separate lipoplexes (0.3 uL/well for each). DNA isolation and NGS analysis were performed as described in Example 1.

TABLE 9
mRNA sequences

| | SEQ | SpyCas9 | | NmeCas9 | Relative orientation |
|---|---|---|---|---|---|
| Construct | ID NO. | moiety | Linker | moiety | of nuclease moieties |
| mRNA A | 1 | R1333K | linker 81aa | WT | R1333K Spy-linker |
| | | Spy | | Nme2 | 81aa-WT Nme2 |
| mRNA N | 46 | WT Spy | linker 81aa | WT | WT Spy-linker 81aa- |
| | | | | Nme2 | WT Nme2 |
| mRNA O | 48 | R1333K | linker 71aa | WT | R1333K Spy-linker |
| | | Spy | | Nme2 | 71aa-WT Nme2 |
| mRNA P | 53 | R1333K | linker 61aa | WT | R1333K Spy-linker |
| | | Spy | | Nme2 | 61aa-WT Nme2 |
| mRNA Q | 58 | R1333K | linker 51aa | WT | R1333K Spy-linker |
| | | Spy | | Nme2 | 51aa-WT Nme2 |
| mRNA R | 63 | R1333K | linker 41aa | WT | R1333K Spy-linker |
| | | Spy | | Nme2 | 41aa-WT Nme2 |
| mRNA S | 68 | R1333K | linker 31aa | WT | R1333K Spy-linker |
| | | Spy | | Nme2 | 31aa-WT Nme2 |
| mRNA T | 73 | R1333K | linker 21aa | WT | R1333K Spy-linker |
| | | Spy | | Nme2 | 21aa-WT Nme2 |
| mRNA U | 78 | R1333K | linker 11aa | WT | R1333K Spy-linker |
| | | Spy | | Nme2 | 11aa-WT Nme2 |
| mRNA V | 83 | Spy | linker 81aa | WT | SpydRec2-linker |
| | | dRec2 | | Nme2 | 81aa-WT Nme2 |
| mRNA W | 88 | Spy | linker 81aa | WT | SpydRec3-linker |
| | | dRec3 | | Nme2 | 81aa-WT Nme2 |

TABLE 10
Orthogonal Cas9-Cas9 fusion linker and Rec domain-
null mRNA and amount transfected in 96-well plates

| mRNA | size relative to SpyCas9 construct | total ng/well |
|---|---|---|
| mRNA E (SpyCas9) | 1 | 100 |
| mRNA A | 1.86 | 186 |
| mRNA N | 1.86 | 186 |
| mRNA O | 1.73 | 173 |
| mRNA P | 1.73 | 173 |
| mRNA Q | 1.72 | 172 |
| mRNA R | 1.71 | 171 |
| mRNA S | 1.71 | 171 |
| mRNA T | 1.70 | 170 |
| mRNA U | 1.69 | 169 |
| mRNA V | 1.65 | 165 |
| mRNA W | 1.59 | 159 |

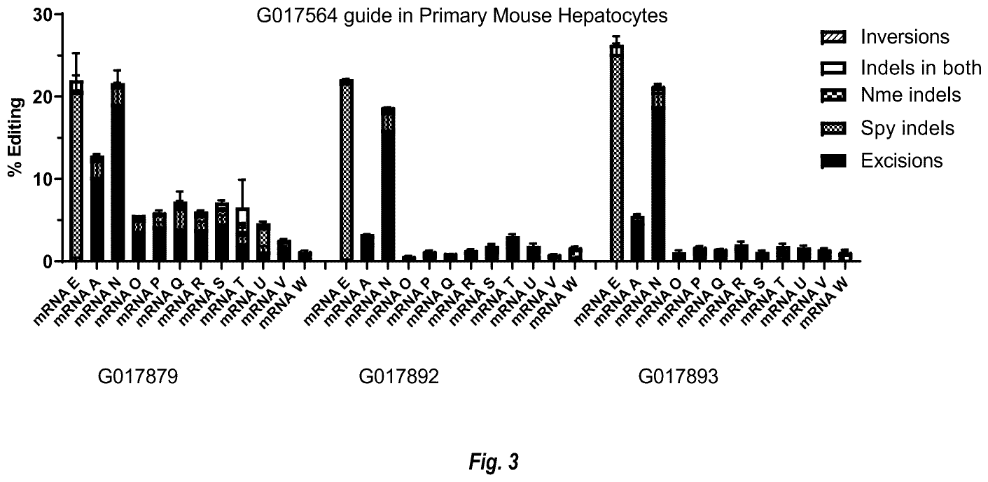

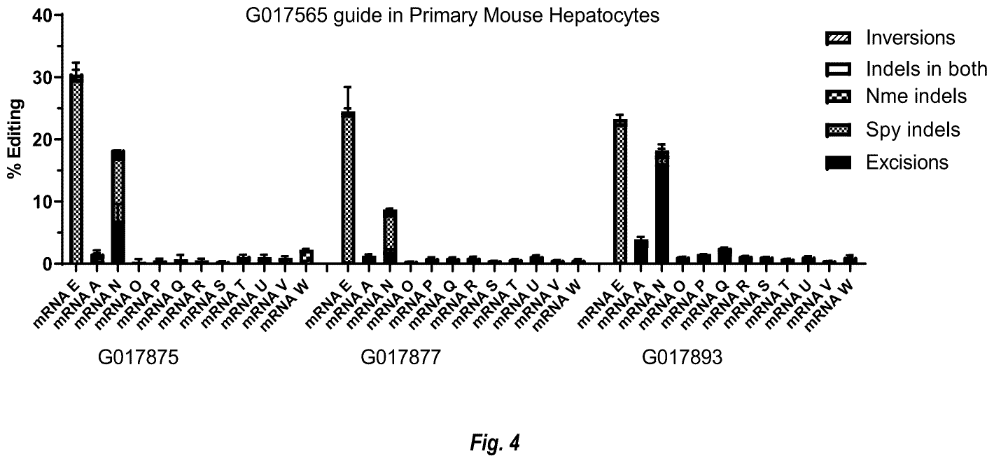

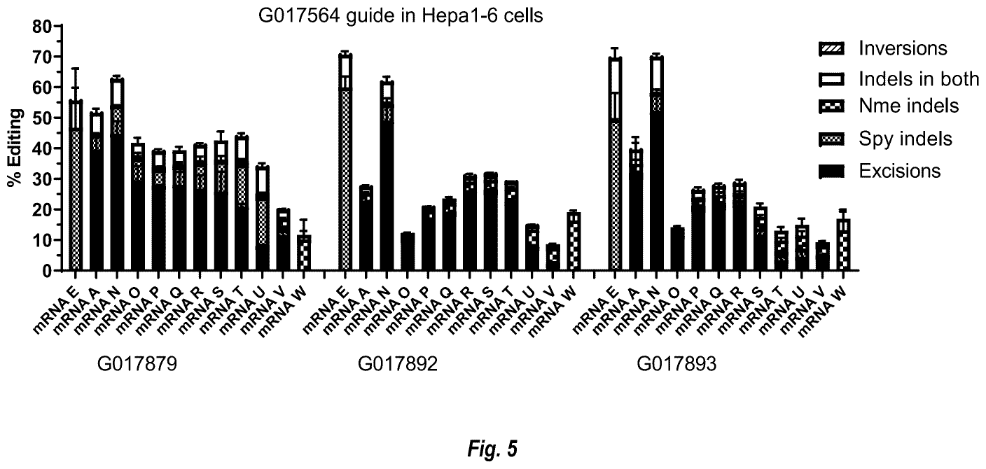

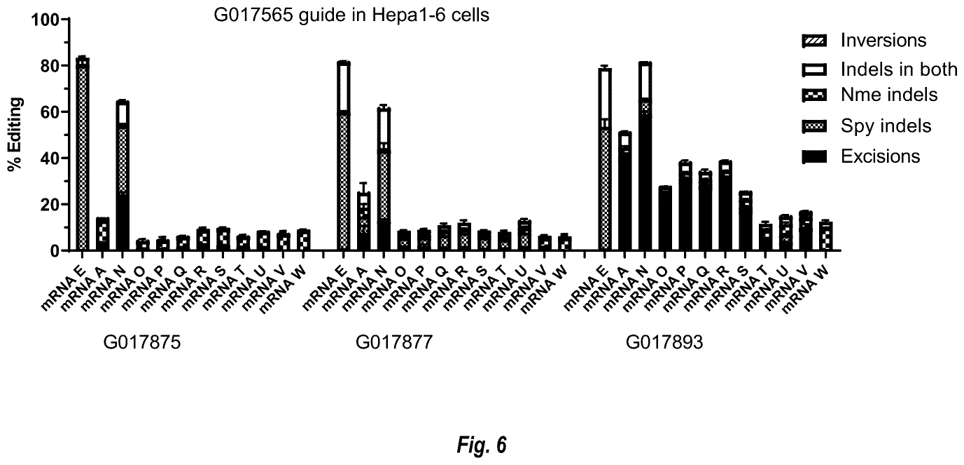

Percent editing and excision at the PCSK9 locus by these guides is shown in Table 11 and FIGS. 3-4 for PMH and in Table 12 and FIGS. 5-6 for Hepa 1-6 cells.

TABLE 11
Percent editing and excision at the PCSK9 locus in PMH (n = 2 for all groups)

| | Spy | Nme | Spy indels | | Nme indels | | Excisions | | Indels in both | | Inversions | |
|---|---|---|---|---|---|---|---|---|---|---|---|---|
| mRNA | Guide | Guide | Mean | SD | Mean | SD | Mean | SD | Mean | SD | Mean | SD |
| mRNA E | G017879 | G017564 | 20.45 | 4.65 | 0.07 | 0.06 | 0.18 | 0.24 | 1.30 | 0.58 | 0.00 | 0.00 |
| mRNA A | | | 2.07 | 0.53 | 0.14 | 0.16 | 10.28 | 1.84 | 0.38 | 0.18 | 0.00 | 0.00 |
| mRNA N | | | 2.15 | 0.31 | 0.05 | 0.00 | 19.17 | 4.02 | 0.29 | 0.05 | 0.00 | 0.00 |
| mRNA O | | | 1.37 | 0.31 | 0.04 | 0.02 | 3.73 | 1.54 | 0.49 | 0.01 | 0.00 | 0.00 |
| mRNA P | | | 1.50 | 0.43 | 0.03 | 0.02 | 4.25 | 0.50 | 0.09 | 0.00 | 0.00 | 0.00 |
| mRNA Q | | | 2.68 | 1.76 | 0.09 | 0.09 | 4.04 | 2.23 | 0.47 | 0.07 | 0.00 | 0.00 |
| mRNA R | | | 1.76 | 0.17 | 0.05 | 0.08 | 3.88 | 0.93 | 0.37 | 0.14 | 0.00 | 0.00 |
| mRNA S | | | 1.76 | 0.09 | 0.05 | 0.00 | 4.62 | 0.97 | 0.73 | 0.26 | 0.00 | 0.00 |
| mRNA T | | | 1.21 | 1.02 | 0.32 | 0.39 | 2.18 | 2.45 | 2.86 | 3.35 | 0.00 | 0.00 |
| mRNA U | | | 2.95 | 0.35 | 0.18 | 0.04 | 1.20 | 0.66 | 0.30 | 0.20 | 0.00 | 0.00 |
| mRNA V | | | 0.59 | 0.03 | 0.65 | 0.27 | 1.20 | 0.14 | 0.14 | 0.02 | 0.00 | 0.00 |
| mRNA W | | | 0.30 | 0.14 | 0.65 | 0.20 | 0.15 | 0.04 | 0.02 | 0.00 | 0.00 | 0.00 |
| mRNA E | G017892 | G017564 | 21.67 | 0.18 | 0.01 | 0.01 | 0.09 | 0.13 | 0.34 | 0.03 | 0.00 | 0.00 |
| mRNA A | | | 0.37 | 0.01 | 0.59 | 0.22 | 2.11 | 0.51 | 0.24 | 0.00 | 0.00 | 0.00 |
| mRNA N | | | 2.15 | 0.56 | 0.01 | 0.01 | 16.02 | 1.96 | 0.39 | 0.01 | 0.12 | 0.00 |
| mRNA O | | | 0.06 | 0.01 | 0.07 | 0.10 | 0.36 | 0.07 | 0.13 | 0.05 | 0.00 | 0.00 |
| mRNA P | | | 0.11 | 0.11 | 0.18 | 0.03 | 0.81 | 0.50 | 0.07 | 0.04 | 0.00 | 0.00 |
| mRNA Q | | | 0.11 | 0.01 | 0.11 | 0.01 | 0.65 | 0.03 | 0.08 | 0.00 | 0.00 | 0.00 |
| mRNA R | | | 0.08 | 0.05 | 0.05 | 0.00 | 1.07 | 0.16 | 0.18 | 0.08 | 0.00 | 0.00 |
| mRNA S | | | 0.14 | 0.01 | 0.20 | 0.05 | 1.27 | 0.85 | 0.26 | 0.06 | 0.00 | 0.00 |
| mRNA T | | | 0.36 | 0.04 | 0.34 | 0.15 | 1.94 | 0.12 | 0.41 | 0.25 | 0.00 | 0.00 |
| mRNA U | | | 0.25 | 0.19 | 0.76 | 0.05 | 0.41 | 0.19 | 0.48 | 0.27 | 0.00 | 0.00 |
| mRNA V | | | 0.01 | 0.00 | 0.55 | 0.15 | 0.16 | 0.08 | 0.06 | 0.08 | 0.01 | 0.00 |
| mRNA W | | | 0.10 | 0.15 | 1.22 | 0.23 | 0.22 | 0.05 | 0.03 | 0.04 | 0.00 | 0.00 |
| mRNA E | G017893 | G017564 | 25.20 | 2.14 | 0.00 | 0.01 | 0.00 | 0.00 | 1.10 | 0.15 | 0.00 | 0.00 |
| mRNA A | | | 0.29 | 0.11 | 0.13 | 0.05 | 4.43 | 1.31 | 0.68 | 0.04 | 0.00 | 0.00 |
| mRNA N | | | 1.78 | 0.51 | 0.01 | 0.00 | 18.85 | 2.71 | 0.49 | 0.22 | 0.06 | 0.00 |
| mRNA O | | | 0.11 | 0.02 | 0.12 | 0.09 | 0.64 | 0.05 | 0.24 | 0.24 | 0.00 | 0.00 |
| mRNA P | | | 0.15 | 0.08 | 0.08 | 0.09 | 1.17 | 0.41 | 0.20 | 0.21 | 0.02 | 0.00 |
| mRNA Q | | | 0.30 | 0.02 | 0.01 | 0.00 | 0.97 | 0.32 | 0.09 | 0.12 | 0.00 | 0.00 |
| mRNA R | | | 0.22 | 0.07 | 0.09 | 0.10 | 1.43 | 0.18 | 0.35 | 0.30 | 0.00 | 0.00 |
| mRNA S | | | 0.25 | 0.11 | 0.17 | 0.07 | 0.50 | 0.17 | 0.20 | 0.19 | 0.00 | 0.00 |
| mRNA T | | | 0.72 | 0.24 | 0.35 | 0.02 | 0.22 | 0.09 | 0.57 | 0.30 | 0.00 | 0.00 |
| mRNA U | | | 0.66 | 0.25 | 0.42 | 0.12 | 0.13 | 0.12 | 0.48 | 0.23 | 0.00 | 0.00 |
| mRNA V | | | 0.22 | 0.02 | 0.39 | 0.07 | 0.69 | 0.14 | 0.13 | 0.15 | 0.00 | 0.00 |
| mRNA W | | | 0.03 | 0.02 | 0.92 | 0.46 | 0.00 | 0.00 | 0.08 | 0.11 | 0.00 | 0.00 |
| mRNA E | G017875 | G017565 | 29.68 | 2.65 | 0.71 | 0.82 | 0.00 | 0.00 | 0.03 | 0.01 | 0.00 | 0.00 |
| mRNA A | | | 0.20 | 0.29 | 0.85 | 0.77 | 0.32 | 0.45 | 0.11 | 0.16 | 0.00 | 0.00 |
| mRNA N | | | 10.14 | 1.08 | 0.32 | 0.03 | 6.99 | 2.59 | 0.67 | 0.10 | 0.06 | 0.00 |
| mRNA O | | | 0.02 | 0.02 | 0.32 | 0.40 | 0.00 | 0.00 | 0.00 | 0.00 | 0.00 | 0.00 |
| mRNA P | | | 0.29 | 0.41 | 0.24 | 0.28 | 0.00 | 0.00 | 0.00 | 0.00 | 0.00 | 0.00 |
| mRNA Q | | | 0.03 | 0.01 | 0.68 | 0.69 | 0.00 | 0.00 | 0.00 | 0.00 | 0.00 | 0.00 |
| mRNA R | | | 0.05 | 0.04 | 0.37 | 0.27 | 0.14 | 0.20 | 0.00 | 0.00 | 0.00 | 0.00 |
| mRNA S | | | 0.01 | 0.01 | 0.15 | 0.05 | 0.17 | 0.24 | 0.00 | 0.00 | 0.00 | 0.00 |
| mRNA T | | | 0.20 | 0.01 | 0.83 | 0.44 | 0.00 | 0.00 | 0.00 | 0.00 | 0.06 | 0.00 |
| mRNA U | | | 0.30 | 0.30 | 0.49 | 0.38 | 0.28 | 0.39 | 0.00 | 0.00 | 0.00 | 0.00 |
| mRNA V | | | 0.07 | 0.06 | 0.87 | 0.28 | 0.00 | 0.00 | 0.00 | 0.00 | 0.00 | 0.00 |
| mRNA W | | | 0.20 | 0.29 | 2.05 | 0.13 | 0.00 | 0.00 | 0.00 | 0.00 | 0.00 | 0.00 |
| mRNA E | G017877 | G017565 | 24.03 | 4.29 | 0.00 | 0.01 | 0.08 | 0.12 | 0.42 | 0.47 | 0.00 | 0.00 |
| mRNA A | | | 0.69 | 0.28 | 0.20 | 0.25 | 0.15 | 0.11 | 0.24 | 0.26 | 0.00 | 0.00 |
| mRNA N | | | 6.02 | 0.68 | 0.16 | 0.11 | 2.01 | 0.30 | 0.53 | 0.14 | 0.00 | 0.00 |
| mRNA O | | | 0.10 | 0.01 | 0.08 | 0.08 | 0.11 | 0.15 | 0.00 | 0.00 | 0.00 | 0.00 |
| mRNA P | | | 0.68 | 0.35 | 0.06 | 0.05 | 0.01 | 0.01 | 0.00 | 0.00 | 0.00 | 0.00 |
| mRNA Q | | | 0.61 | 0.18 | 0.04 | 0.04 | 0.03 | 0.04 | 0.14 | 0.19 | 0.00 | 0.00 |
| mRNA R | | | 0.60 | 0.54 | 0.13 | 0.08 | 0.00 | 0.00 | 0.17 | 0.23 | 0.00 | 0.00 |
| mRNA S | | | 0.28 | 0.04 | 0.02 | 0.02 | 0.00 | 0.00 | 0.17 | 0.01 | 0.00 | 0.00 |
| mRNA T | | | 0.39 | 0.08 | 0.04 | 0.04 | 0.06 | 0.07 | 0.11 | 0.13 | 0.00 | 0.00 |
| mRNA U | | | 0.75 | 0.50 | 0.19 | 0.13 | 0.04 | 0.05 | 0.17 | 0.18 | 0.00 | 0.00 |
| mRNA V | | | 0.04 | 0.03 | 0.28 | 0.18 | 0.07 | 0.10 | 0.09 | 0.12 | 0.00 | 0.00 |
| mRNA W | | | 0.01 | 0.01 | 0.38 | 0.38 | 0.00 | 0.00 | 0.13 | 0.19 | 0.00 | 0.00 |
| mRNA E | G017893 | G017565 | 22.52 | 0.34 | 0.00 | 0.01 | 0.07 | 0.10 | 0.68 | 0.69 | 0.00 | 0.00 |
| mRNA A | | | 0.31 | 0.24 | 0.07 | 0.04 | 3.14 | 0.60 | 0.43 | 0.37 | 0.00 | 0.00 |
| mRNA N | | | 1.23 | 0.40 | 0.03 | 0.02 | 16.15 | 3.06 | 0.83 | 0.29 | 0.01 | 0.00 |
| mRNA O | | | 0.11 | 0.08 | 0.06 | 0.03 | 0.75 | 0.02 | 0.10 | 0.11 | 0.00 | 0.00 |
| mRNA P | | | 0.18 | 0.04 | 0.02 | 0.02 | 0.89 | 0.32 | 0.43 | 0.00 | 0.00 | 0.00 |
| mRNA Q | | | 0.40 | 0.01 | 0.18 | 0.20 | 1.73 | 0.38 | 0.21 | 0.09 | 0.00 | 0.00 |
| mRNA R | | | 0.31 | 0.23 | 0.04 | 0.03 | 0.68 | 0.56 | 0.12 | 0.01 | 0.00 | 0.00 |
| mRNA S | | | 0.34 | 0.07 | 0.09 | 0.11 | 0.47 | 0.01 | 0.12 | 0.07 | 0.00 | 0.00 |
| mRNA T | | | 0.40 | 0.14 | 0.19 | 0.04 | 0.01 | 0.01 | 0.16 | 0.03 | 0.00 | 0.00 |
| mRNA U | | | 0.46 | 0.10 | 0.32 | 0.31 | 0.12 | 0.09 | 0.06 | 0.04 | 0.00 | 0.00 |
| mRNA V | | | 0.07 | 0.09 | 0.11 | 0.01 | 0.26 | 0.07 | 0.01 | 0.00 | 0.00 | 0.00 |
| mRNA W | | | 0.02 | 0.01 | 0.83 | 0.48 | 0.00 | 0.00 | 0.03 | 0.04 | 0.00 | 0.00 |

TABLE 12
Percent editing and excision at the PCSK9 locus in Hepa 1-6 cells (n = 2 for all groups)

| | | | Spy indels | | Nme indels | | Excisions | | Indels in both | | Inversions | |
|---|---|---|---|---|---|---|---|---|---|---|---|---|
| mRNA | Spy Guide | Nme Guide | Mean | SD | Mean | SD | Mean | SD | Mean | SD | Mean | SD |
| mRNA E | G017879 | G017564 | 46.64 | 19.31 | 0.01 | 0.01 | 0.12 | 0.11 | 8.87 | 4.17 | 0.04 | 0.00 |
| mRNA A | | | 4.71 | 0.20 | 0.63 | 0.03 | 39.61 | 4.09 | 6.62 | 1.39 | 0.10 | 0.00 |
| mRNA N | | | 9.14 | 0.24 | 0.17 | 0.03 | 44.75 | 4.08 | 8.59 | 1.03 | 0.12 | 0.00 |
| mRNA O | | | 7.12 | 1.82 | 0.65 | 0.11 | 29.54 | 4.60 | 4.44 | 1.68 | 0.01 | 0.00 |
| mRNA P | | | 5.50 | 0.41 | 0.79 | 0.13 | 27.63 | 0.23 | 5.14 | 0.68 | 0.04 | 0.00 |
| mRNA Q | | | 5.97 | 1.37 | 0.73 | 0.32 | 28.07 | 4.23 | 4.59 | 1.15 | 0.03 | 0.00 |
| mRNA R | | | 8.23 | 2.25 | 0.71 | 0.06 | 26.80 | 4.39 | 5.37 | 0.54 | 0.07 | 0.00 |
| mRNA S | | | 9.89 | 1.79 | 0.62 | 0.01 | 25.85 | 6.53 | 6.19 | 2.93 | 0.04 | 0.00 |
| mRNA T | | | 13.78 | 1.77 | 1.02 | 0.36 | 20.94 | 0.89 | 8.09 | 1.07 | 0.15 | 0.00 |
| mRNA U | | | 16.08 | 1.00 | 1.09 | 0.34 | 7.99 | 0.47 | 8.84 | 1.08 | 0.07 | 0.00 |
| mRNA V | | | 2.48 | 0.27 | 2.93 | 0.23 | 11.60 | 1.08 | 2.91 | 0.27 | 0.03 | 0.00 |
| mRNA W | | | 0.03 | 0.03 | 10.13 | 6.41 | 0.05 | 0.03 | 1.46 | 1.26 | 0.05 | 0.00 |
| mRNA E | G017892 | G017564 | 59.89 | 3.57 | 0.00 | 0.00 | 0.06 | 0.01 | 10.71 | 1.09 | 0.03 | 0.00 |
| mRNA A | | | 0.57 | 0.25 | 3.05 | 0.20 | 21.72 | 3.44 | 2.08 | 0.50 | 0.11 | 0.00 |
| mRNA N | | | 5.24 | 0.65 | 0.10 | 0.05 | 49.10 | 7.28 | 7.35 | 1.64 | 0.14 | 0.00 |
| mRNA O | | | 0.30 | 0.06 | 1.34 | 0.19 | 10.17 | 0.06 | 0.47 | 0.18 | 0.03 | 0.00 |
| mRNA P | | | 0.41 | 0.06 | 2.64 | 0.09 | 16.51 | 2.97 | 1.24 | 0.15 | 0.10 | 0.00 |
| mRNA Q | | | 0.49 | 0.00 | 2.79 | 1.07 | 18.80 | 3.65 | 1.46 | 0.50 | 0.05 | 0.00 |
| mRNA R | | | 0.59 | 0.00 | 2.79 | 0.66 | 25.73 | 4.04 | 1.92 | 0.61 | 0.04 | 0.00 |
| mRNA S | | | 0.65 | 0.14 | 3.15 | 0.57 | 25.81 | 3.18 | 2.06 | 0.34 | 0.11 | 0.00 |
| mRNA T | | | 0.62 | 0.03 | 4.14 | 0.39 | 22.19 | 1.14 | 2.16 | 0.06 | 0.05 | 0.00 |
| mRNA U | | | 0.55 | 0.06 | 5.25 | 0.84 | 7.60 | 0.71 | 1.37 | 0.29 | 0.06 | 0.00 |
| mRNA V | | | 0.09 | 0.02 | 5.25 | 1.13 | 2.12 | 0.58 | 1.17 | 0.12 | 0.01 | 0.00 |
| mRNA W | | | 0.01 | 0.01 | 16.38 | 1.91 | 0.05 | 0.05 | 2.63 | 0.56 | 0.00 | 0.00 |
| mRNA E | G017893 | G017564 | 49.29 | 8.63 | 0.01 | 0.01 | 0.16 | 0.13 | 20.10 | 3.20 | 0.12 | 0.00 |
| mRNA A | | | 1.09 | 0.41 | 2.97 | 1.28 | 30.89 | 12.84 | 4.60 | 2.21 | 0.09 | 0.00 |
| mRNA N | | | 5.68 | 1.27 | 0.12 | 0.06 | 52.28 | 5.43 | 11.79 | 1.10 | 0.14 | 0.00 |
| mRNA O | | | 0.54 | 0.05 | 1.26 | 0.28 | 11.29 | 2.40 | 1.08 | 0.40 | 0.01 | 0.00 |
| mRNA P | | | 0.93 | 0.20 | 2.46 | 0.07 | 20.21 | 2.51 | 2.72 | 0.91 | 0.09 | 0.00 |
| mRNA Q | | | 1.17 | 0.25 | 2.67 | 0.82 | 20.94 | 6.10 | 2.90 | 0.85 | 0.08 | 0.00 |
| mRNA R | | | 1.30 | 0.61 | 2.27 | 0.55 | 21.75 | 6.06 | 3.32 | 1.02 | 0.06 | 0.00 |
| mRNA S | | | 2.02 | 0.74 | 3.77 | 0.54 | 11.75 | 3.27 | 3.42 | 1.04 | 0.02 | 0.00 |
| mRNA T | | | 2.57 | 0.40 | 3.83 | 0.83 | 3.46 | 0.99 | 3.19 | 1.21 | 0.00 | 0.00 |
| mRNA U | | | 2.70 | 0.90 | 4.30 | 1.34 | 4.52 | 1.78 | 3.43 | 2.09 | 0.02 | 0.00 |
| mRNA V | | | 0.22 | 0.03 | 3.08 | 0.74 | 4.81 | 0.56 | 1.18 | 0.35 | 0.01 | 0.00 |
| mRNA W | | | 0.01 | 0.01 | 13.13 | 6.68 | 0.12 | 0.02 | 3.69 | 2.34 | 0.00 | 0.00 |
| mRNA E | G017875 | G017565 | 80.13 | 3.74 | 0.02 | 0.02 | 0.10 | 0.12 | 3.11 | 0.15 | 0.00 | 0.00 |
| mRNA A | | | 0.39 | 0.18 | 9.61 | 0.18 | 2.99 | 0.58 | 0.91 | 0.07 | 0.06 | 0.00 |
| mRNA N | | | 29.68 | 0.50 | 0.72 | 0.04 | 24.38 | 0.85 | 9.55 | 0.72 | 0.23 | 0.00 |
| mRNA O | | | 0.08 | 0.02 | 3.22 | 0.81 | 0.82 | 0.09 | 0.07 | 0.02 | 0.00 | 0.00 |
| mRNA P | | | 0.13 | 0.09 | 3.43 | 1.61 | 0.69 | 0.06 | 0.27 | 0.29 | 0.02 | 0.00 |
| mRNA Q | | | 0.25 | 0.11 | 4.60 | 0.11 | 1.05 | 0.53 | 0.31 | 0.17 | 0.01 | 0.00 |
| mRNA R | | | 0.29 | 0.12 | 6.53 | 1.18 | 1.97 | 0.86 | 0.48 | 0.26 | 0.02 | 0.00 |
| mRNA S | | | 0.37 | 0.01 | 6.99 | 0.05 | 1.97 | 0.37 | 0.47 | 0.28 | 0.00 | 0.00 |
| mRNA T | | | 0.18 | 0.01 | 4.49 | 0.88 | 1.27 | 0.48 | 0.30 | 0.13 | 0.00 | 0.00 |
| mRNA U | | | 0.26 | 0.20 | 6.66 | 0.21 | 1.09 | 0.37 | 0.33 | 0.05 | 0.00 | 0.00 |
| mRNA V | | | 0.02 | 0.02 | 6.99 | 1.45 | 0.02 | 0.02 | 0.30 | 0.27 | 0.02 | 0.00 |
| mRNA W | | | 0.04 | 0.02 | 8.47 | 0.20 | 0.00 | 0.00 | 0.42 | 0.23 | 0.00 | 0.00 |
| mRNA E | G017877 | G017565 | 59.48 | 0.52 | 0.01 | 0.00 | 0.32 | 0.25 | 21.60 | 0.48 | 0.02 | 0.00 |
| mRNA A | | | 7.16 | 2.91 | 3.83 | 1.43 | 7.67 | 3.03 | 6.33 | 4.22 | 0.06 | 0.00 |
| mRNA N | | | 30.46 | 3.12 | 0.41 | 0.07 | 12.88 | 0.70 | 17.72 | 1.52 | 0.10 | 0.00 |
| mRNA O | | | 3.80 | 0.36 | 1.41 | 0.11 | 1.87 | 0.05 | 1.52 | 0.30 | 0.01 | 0.00 |
| mRNA P | | | 3.86 | 0.74 | 1.24 | 0.19 | 1.84 | 0.77 | 2.06 | 0.35 | 0.00 | 0.00 |
| mRNA Q | | | 5.41 | 0.76 | 1.89 | 0.23 | 1.28 | 0.10 | 2.41 | 0.70 | 0.01 | 0.00 |
| mRNA R | | | 6.63 | 0.71 | 1.52 | 0.29 | 1.19 | 0.24 | 2.73 | 1.06 | 0.00 | 0.00 |
| mRNA S | | | 5.17 | 0.19 | 1.17 | 0.32 | 0.61 | 0.09 | 1.75 | 0.13 | 0.00 | 0.00 |
| mRNA T | | | 4.31 | 0.58 | 1.31 | 0.23 | 0.62 | 0.32 | 1.84 | 0.52 | 0.00 | 0.00 |
| mRNA U | | | 6.63 | 1.17 | 1.86 | 0.54 | 1.28 | 0.15 | 2.98 | 0.98 | 0.01 | 0.00 |
| mRNA V | | | 1.28 | 0.03 | 3.22 | 0.71 | 0.85 | 0.05 | 1.04 | 0.33 | 0.00 | 0.00 |
| mRNA W | | | 0.01 | 0.01 | 5.44 | 1.64 | 0.01 | 0.02 | 0.76 | 0.05 | 0.00 | 0.00 |
| mRNA E | G017893 | G017565 | 53.15 | 3.21 | 0.00 | 0.00 | 0.39 | 0.05 | 25.32 | 1.08 | 0.02 | 0.00 |
| mRNA A | | | 1.23 | 0.15 | 3.68 | 0.62 | 39.60 | 2.48 | 6.43 | 0.61 | 0.16 | 0.00 |
| mRNA N | | | 6.47 | 0.31 | 0.08 | 0.07 | 58.93 | 1.19 | 15.70 | 0.25 | 0.19 | 0.00 |
| mRNA O | | | 0.88 | 0.02 | 1.58 | 0.06 | 22.63 | 2.76 | 2.34 | 0.29 | 0.07 | 0.00 |
| mRNA P | | | 1.06 | 0.25 | 2.69 | 0.02 | 30.23 | 1.26 | 4.10 | 0.86 | 0.07 | 0.00 |
| mRNA Q | | | 1.08 | 0.09 | 2.09 | 0.33 | 27.52 | 2.30 | 3.24 | 1.10 | 0.10 | 0.00 |
| mRNA R | | | 1.36 | 0.05 | 2.50 | 0.14 | 30.57 | 0.55 | 3.99 | 0.55 | 0.09 | 0.00 |
| mRNA S | | | 1.65 | 0.23 | 3.21 | 0.14 | 17.30 | 0.21 | 3.15 | 0.14 | 0.07 | 0.00 |
| mRNA T | | | 2.24 | 0.04 | 3.37 | 0.47 | 3.14 | 0.50 | 2.78 | 0.96 | 0.04 | 0.00 |
| mRNA U | | | 3.13 | 0.34 | 4.60 | 0.72 | 3.76 | 0.05 | 3.22 | 0.63 | 0.05 | 0.00 |
| mRNA V | | | 0.30 | 0.16 | 4.43 | 0.47 | 9.50 | 1.90 | 2.42 | 0.47 | 0.02 | 0.00 |
| mRNA W | | | 0.02 | 0.02 | 9.84 | 0.06 | 0.05 | 0.03 | 2.57 | 0.66 | 0.00 | 0.00 |

Amino acid linkers were tested for expression efficacy in Hepa1-6 cells. Hepa1-6 cells were cultured in DMEM media supplemented with 10% fetal bovine serum at a density of 375,000 cells/well in a 6-well plate. Plated cells were agitated by shaking by hand every 15 minutes for 1 hour to ensure even distribution and then were allowed to settle and adhere overnight prior to transfection. Cells were transfected using 7.5 uL/well Lipofectamine Messenger Max (ThermoFisher, Cat. LMRNA001) diluted in OptiMem Medium (ThermoFisher, Cat. 31985062) according to the manufacturer's protocol, with, in each well, 2,500 ng SpyCas9 mRNA equivalents of a nuclease or orthogonal Cas9-Cas9 fusion construct as described in Table 13.

TABLE 13
Orthogonal Cas9-Cas9 fusion linker and Rec domain-null
mRNA and amount (ng) transfected in 6-well plates

| mRNA | size relative to Spy | total ng/well |
|---|---|---|
| mRNA A | 1.86 | 4640 |
| mRNA E (SpyCas9) | 1 | 2500 |
| mRNA N | 1.86 | 4640 |
| mRNA O | 1.73 | 4331 |
| mRNA P | 1.73 | 4314 |
| mRNA Q | 1.72 | 4297 |
| mRNA R | 1.71 | 4280 |
| mRNA S | 1.71 | 4263 |
| mRNA T | 1.70 | 4246 |
| mRNA U | 1.69 | 4229 |
| mRNA V | 1.65 | 4128 |
| mRNA W | 1.59 | 3986 |

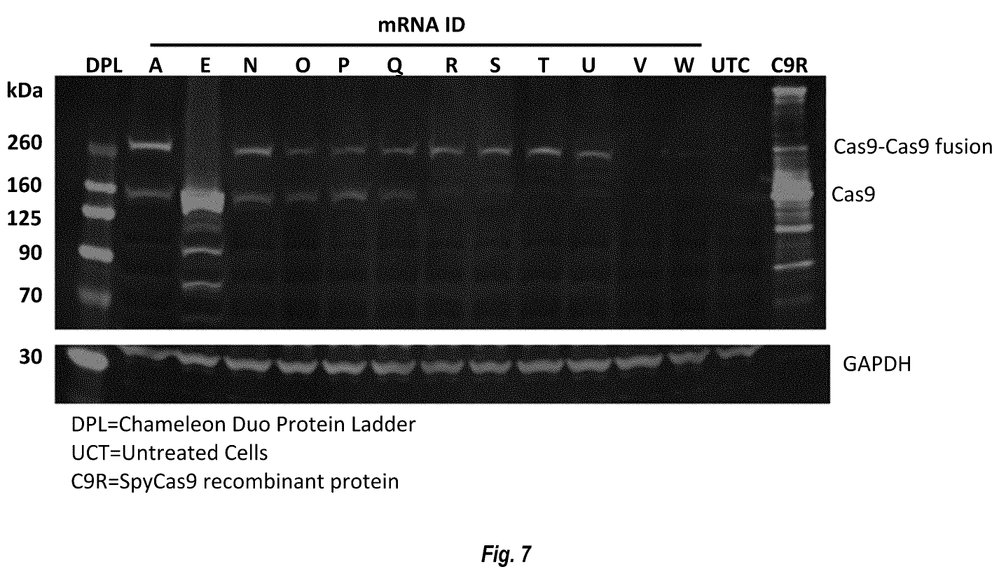

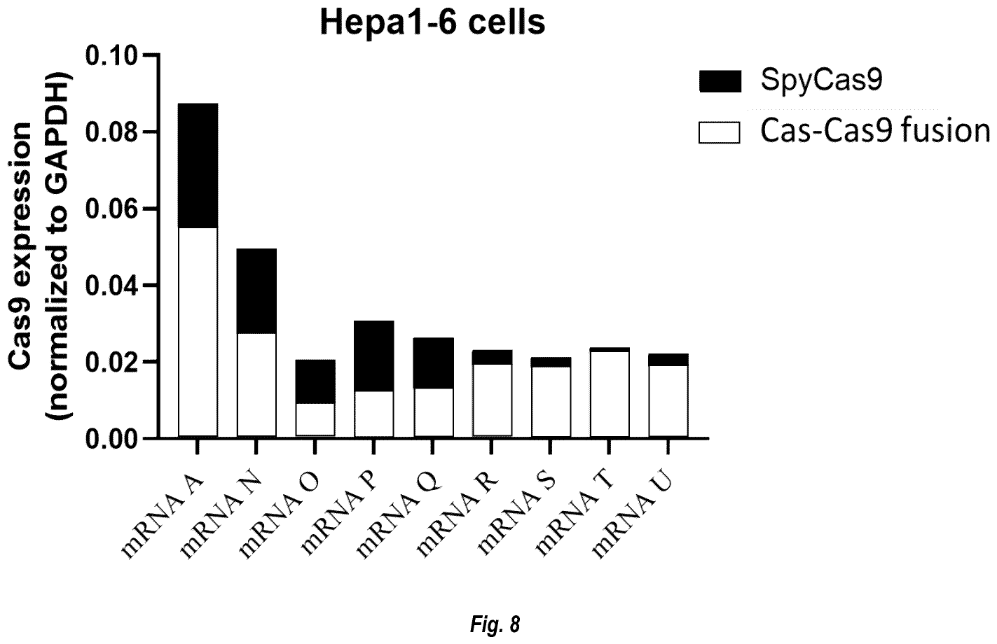

Protein isolation and western blot analysis were performed as follows to measure orthogonal Cas9-Cas9 fusion and SpyCas9 expression in transfected cells. 72 hours post-treatment, cell plates were placed on ice and washed once with cold PBS, and then 60 uL/well of RIPA Lysis Buffer (Boston Bioproducts, Cat. BP-115) supplemented with 1× Protease Inhibitor (ThermoFisher, Cat. 1861278), 0.01M DTT (Cat. P2325), and 1× Benzonase Nuclease (EMD Millipore, Cat. 71206-25KUN) was added. Cells were scraped, lysates were pipetted up and down and left to incubate five minutes on ice. Lysates were moved to an Eppendorf tube and spun down in a tabletop centrifuge at 4° C. for 15 minutes at 13,000×g. Supernatant was collected in a fresh Eppendorf tube and protein was quantified using Pierce Coomassie Plus (Bradford) Assay Reagent (ThermoFisher, Cat. 23238) as follows. Pierce Coomassie Plus Protein Assay Reagent was brought to room temperature. 2 mg/mL albumin standard was serially diluted in equal volumes of PBS. Cell lysates were diluted 5-fold in PBS (4 uL cell lysate in 16 uL PBS). 5 μL of each standard or cell lysate sample was pipetted into a clear bottom polystyrene 96-well flat bottom plate in triplicates. 150 μL of Coomassie Plus Protein Assay Reagent was added to each well and mixed with a plate shaker for 30 seconds at room temperature. Plates were removed from the shaker and incubated for 10 minutes at room temperature. The absorbance at 595 nm was measured with a plate reader. Total protein quantification was used to calculate 40 ug/lane of each protein sample to run in a Novex WedgeWell 4-20% Tris-Glycine Gel (ThermoFisher, Cat. XP04205BOX) with 20× NUPAGE Tris-Acetate Running Buffer (ThermoFisher, Cat. LA0041). Samples were run on the gel for 1 hour at 150 volts with Chameleon Duo Pre-stained Protein Ladder (Licor, Cat. 928-60000) and transferred to a 0.45 m Nitrocellulose Membrane (ThermoFisher, Cat. 77010). The membrane was blocked in LiCor Odyssey Blocking Buffer (TBS) (LiCor, Cat. 927-60001) for 1 hour and incubated overnight at 4° C. with antibodies against SpyCas9 (Daigenode, Cat. 15310258) and GAPDH (GeneTex, Cat. GTX627408-01) diluted in 1:1 TBST and LiCor Blocking Buffer. The next morning, blots were washed in 1×TBST and incubated for 1 hour at room temperature with goat anti-rabbit IgG (LiCor, Cat. 926-32211) and goat anti-mouse (LiCor, Cat. 926-68070) diluted in 1:1 LiCor Blocking Buffer and TBST and imaged on the LiCor Odyssey Imager (FIG. 7). Truncated SpyCas9 products at 160 kDa were seen in lysates of cells transfected with mRNA A, mRNA N, mRNA O, mRNA P, mRNA Q. This 160 kDa truncated Spy product was reduced in lysates of cells transfected with mRNA R, mRNA S, mRNA T, and mRNA U, and the majority of the protein product is full-length orthogonal Cas9-Cas9 fusion. Results of the Western blot are shown FIG. 7, quantified in Table 14, and illustrated in FIG. 8.

TABLE 14
orthogonal Cas9-Cas9 fusion and SpyCas9 protein expression
in transfected Hepa 1-6 cells, normalized to GAPDH control.

| | Orthogonal Cas9-Cas9 fusion | SpyCas9 Signal |
|---|---|---|
| MRNA | Signal Normalized to GAPDH | Normalized to GAPDH |
| mRNA A | 0.06 | 0.03 |
| mRNA N | 0.03 | 0.02 |
| mRNA O | 0.01 | 0.01 |
| mRNA P | 0.01 | 0.02 |
| mRNA Q | 0.01 | 0.01 |
| mRNA R | 0.02 | 0.00 |
| mRNA S | 0.02 | 0.00 |
| mRNA T | 0.02 | 0.00 |
| mRNA U | 0.02 | 0.00 |

### Example 5—Orthogonal Cas9-Cas9 Fusion LNP Delivery with Nme:Spy Guide Ratio of 1:2 in PMH

Additional orthogonal Cas9-Cas9 fusion mRNA constructs were tested for excision efficacy with different LNP delivery conditions and different Nme:Spy guide ratios, in primary mouse hepatocytes (PMH) at the TTR locus.

PMH (Gibco, MCM882) were thawed and resuspended in 50 mL Cryopreserved Hepatocyte Recovery Media (CHRM) (Invitrogen, CM7000) followed by centrifugation. Cells were resuspended in hepatocyte medium with plating supplements: William's E Medium Plating Supplements with FBS content (Gibco, Cat. A13450). Cells were pelleted by centrifugation, resuspended in media and plated at a density of 15,000 cells/well on Bio-coat collagen I coated 96-well plates (Corning #354407). Plated cells were allowed to settle and adhere overnight in a tissue culture incubator at 37° C. and 5% CO₂ atmosphere. The next morning, cells were checked for monolayer formation, existing media was aspirated and cells were plated with 100 uL Cellartis Power Primary HEP Media (Takara, Cat. Y20020).

Cells were transfected with 2 LNPs prepared as described in Example 1, with a molar ratio of 50% Lipid A, 38% cholesterol, 9% DSPC, and 3% PEG2k-DMG, and delivered in Cellartis Power Primary HEP Media (Takara, Cat. Y20020). One LNP comprised an orthogonal Cas9-Cas9 fusion mRNA construct comprising any of SEQ ID NOs: 1 and 8 and the other LNP comprised both a SpyCas9 guide (SEQ ID NO: 201) and a NmeCas9 guide (SEQ ID NO: 252 or 253) in a weight ratio of Nme:Spy guide of 1:2. The volume ratio of gRNA:mRNA transfected to the cells was 2:1. Each well contained 10 ng of total RNA cargo, with 1.43 ng of orthogonal Cas9-Cas9 fusion mRNA, 5.72 ng SpyCas9 guide and 2.86 ng NmeCas9 guide. The orthogonal Cas9-Cas9 fusion constructs are described in Table 15.

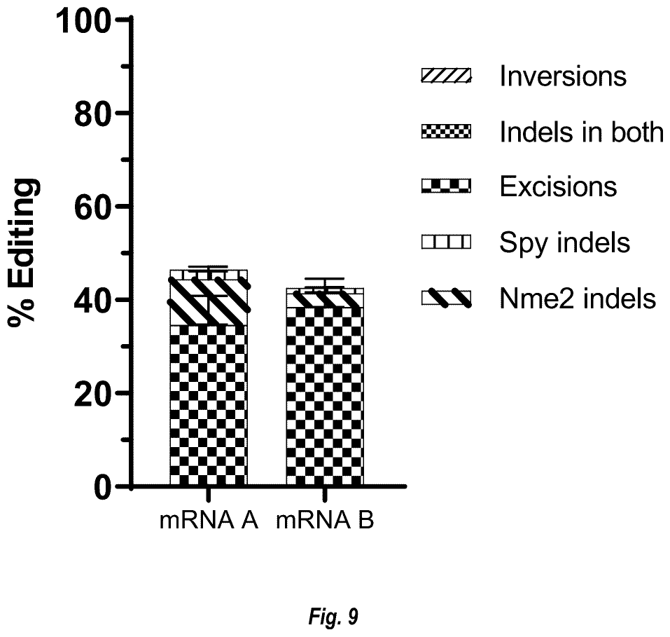

DNA isolation and NGS analysis were performed as described in Example 1. Percent editing and excision at the TTR locus in the transfected cells are shown in Table 16 and illustrated in FIG. 9.

TABLE 15
mRNA sequences

| | SEQ | SpyCas9 | | NmeCas9 | Relative orientation |
|---|---|---|---|---|---|
| Construct | ID NO. | moiety | Linker | moiety | of nuclease moieties |
| mRNA A | 1 | R1333K | linker | WT | R1333K Spy-linker 81aa- |
| | | Spy | 81aa | Nme2 | WT Nme2-SV40NLS |
| mRNA B | 8 | R1333K | linker | WT | SV40NLS-WT Nme2- |
| | | Spy | 41aa | Nme2 | linker 41aa-R1333K Spy- |
| | | | | | SV40NLS-HiBit |

TABLE 16
Editing at the TTR locus as a percent of total
reads in Primary mouse hepatocytes (n = 2)

| | Nme2 indels | | Spy indels | | Excisions | | Indels in both | | Inversions | |
|---|---|---|---|---|---|---|---|---|---|---|
| mRNA | Mean | SD | Mean | SD | Mean | SD | Mean | SD | Mean | SD |
| mRNA A | 9.74 | 1.78 | 2.11 | 0.70 | 34.54 | 6.32 | 0.06 | 0.08 | 0.00 | 0.00 |
| mRNA B | 2.97 | 0.26 | 1.14 | 0.13 | 38.40 | 6.16 | 0.03 | 0.05 | 0.00 | 0.00 |

### Example 6—In Vivo Editing with Orthogonal Cas9-Cas9 Fusion Constructs in Mice

Two types of LNPs were formulated as described in Example 1, with a molar ratio of 50% Lipid A, 38% cholesterol, 9% DSPC, and 3% PEG2k-DMG. LNP1 designates LNPs that comprised two guide RNAs targeting the mouse TTR gene start codon, including one Nme2 sgRNA and one Spy sgRNA at a ratio of 1:2 Nme:Spy sgRNA by weight. LNP2 designates a second type of LNPs that comprised only mRNA. Transport and storage solution (TSS) used in LNP preparation was dosed in the experiment as a vehicle-only negative control.

orthogonal Cas9-Cas9 fusion systems (Spy sgRNA+Nme2 sgRNA+orthogonal Cas9-Cas9 fusion mRNA) were tested for editing efficiency in vivo using LNP1 and LNP2 species as described above. CD-1 female mice (N=5 animals in each group) about 6 weeks of age were used in the experiment and were weighed pre-dose. Animals received a lateral tail vein injection at a dose of 0.3 milligrams per kilogram body weight (e.g., 0.3 mg/kg, or 0.3 mpk) or 1 mg/kg of an LNP mixture comprising a ratio of 2:1 LNP1:LNP2 by volume. The animals were observed at approximately 24 hours post dose for adverse effects. Animals were euthanized 11 days post dose by cardiac exsanguination under isoflurane anesthesia and cervical dislocation. Blood was collected via cardiac puncture into serum separator tubes or into tubes containing buffered sodium citrate for plasma. Liver tissue was collected from the left lateral lobe from each animal for DNA extraction and NGS analysis as described in Example 1. Editing percentages are shown in Table 17 and illustrated in FIG. 10.

TABLE 17
Editing percentages in liver tissue.

| Group | Guide 1 | Guide 2 | | Dose | % Guide1 | | % Guide2 | | % | | % indels | | % | | |
|---|---|---|---|---|---|---|---|---|---|---|---|---|---|---|---|
| # | (Nme2) | (Spy) | mRNA | (mg/kg) | (Spy) | SD | (Nme2) | SD | Excisions | SD | in both | SD | Inversions | SD | N |
| 1 | TSS | X | X | X | 0.0 | 0.0 | 0.0 | 0.0 | 0.1 | 0.1 | 0.0 | 0 | 0.0 | 0 | 5 |
| 2 | G021846 | G000502 | mRNA | 0.3 | 3.1 | 0.4 | 2.2 | 0.2 | 56.8 | 3.7 | 0.5 | 0.2 | 0.0 | 0 | 5 |
| 3 | | | A | 1.0 | 4.4 | 0.5 | 1.9 | 0.2 | 60.7 | 3.0 | 0.8 | 0.1 | 0.0 | 0 | 5 |
| 4 | G023420 | | | 0.3 | 4.2 | 0.6 | 3.9 | 0.7 | 52.8 | 2.1 | 0.7 | 0.4 | 0.0 | 0 | 5 |
| 5 | | | | 1.0 | 5.6 | 0.8 | 3.0 | 0.5 | 55.2 | 2.9 | 1.2 | 0.6 | 0.0 | 0 | 5 |
| 6 | G21846 | | mRNA | 0.3 | 1.1 | 0.1 | 2.0 | 0.4 | 52.8 | 0.9 | 0.4 | 0.1 | 0.0 | 0.1 | 5 |
| 7 | | | B | 1.0 | 1.5 | 0.1 | 1.6 | 0.1 | 60.9 | 3.1 | 0.7 | 0.3 | 0.0 | 0 | 5 |
| 8 | G023420 | | | 0.3 | 1.0 | 0.2 | 3.8 | 0.5 | 49.3 | 6.6 | 0.4 | 0.1 | 0.0 | 0 | 5 |
| 9 | | | | 1.0 | 1.7 | 0.3 | 3.8 | 0.4 | 56.6 | 1.4 | 0.9 | 0.1 | 0.0 | 0.0 | 5 |

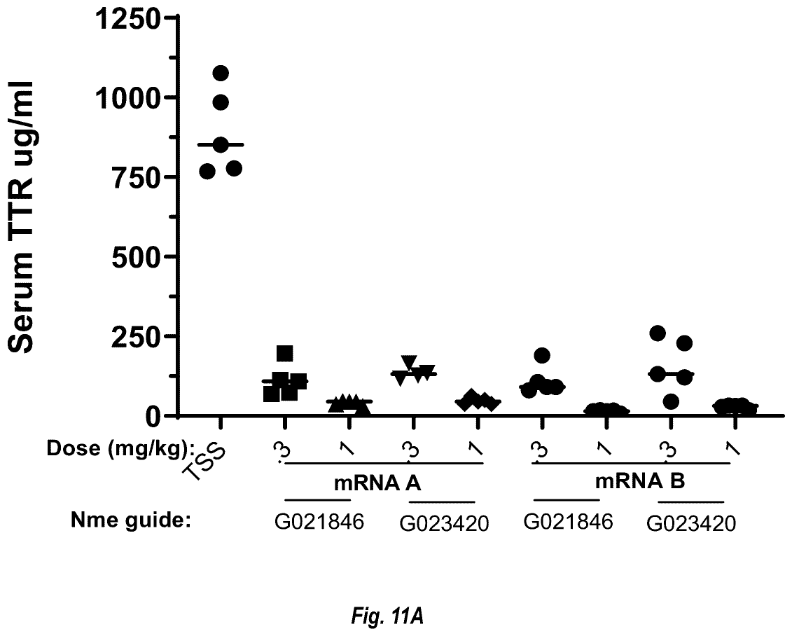

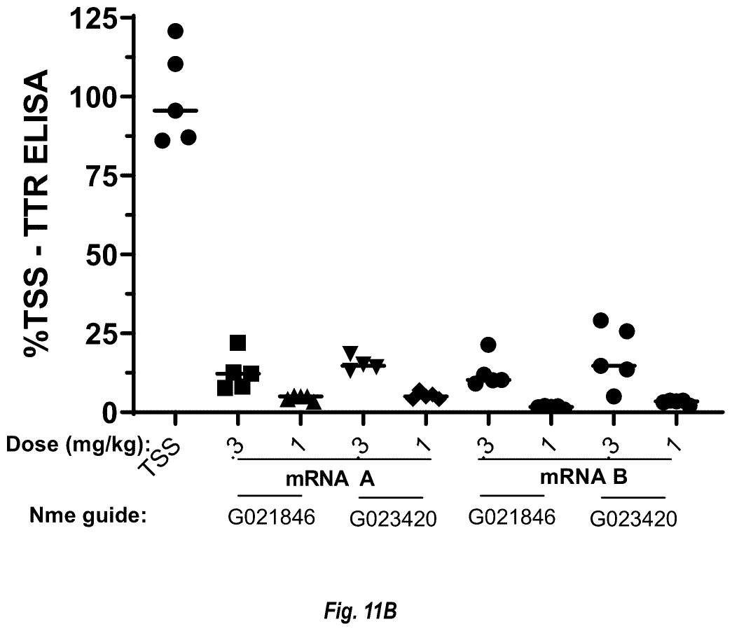

The total serum TTR levels were determined using a Prealbumin (TTR) ELISA Kit, Mouse (Aviva Systems; cat #OKIA00111) (N=5 animals in each group). Kit reagents and standards were prepared according to the manufacturer's protocol. Mouse serum was diluted between 10,000 to 50,000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 or Clariostar plate reader at an absorbance of 450 nm. Serum TTR levels were calculated by SoftMax Pro software ver. 6.4.2 or Mars software ver. 3.31 using a four-parameter logistic curve fit off the standard curve. Final serum values were adjusted for the assay dilution. Serum TTR levels compared to the control group, which consisted of animals sham-treated with vehicle (TSS), are shown in Table 18 and illustrated in FIGS. 11A and 11B.

TABLE 18
Serum TTR analysis.

| | | | | | Serum | | | |
|---|---|---|---|---|---|---|---|---|
| Group | Guide 1 | Guide 2 | | Dose | TTR | SD | | % |
| # | (Nme2) | (Spy) | mRNA | (mg/kg) | (ug/ml) | (ug/ml) | N | TSS |
| 1 | TSS | X | X | X | 891.8 | 135.0 | 5 | 100.0 |
| 2 | G021846 | G000502 | mRNA | 0.3 | 112.2 | 51.2 | 5 | 12.6 |
| 3 | | | A | 1.0 | 40.9 | 7.0 | 5 | 4.6 |
| 4 | G023420 | | | 0.3 | 136.3 | 20.6 | 5 | 15.3 |
| 5 | | | | 1.0 | 46.8 | 10.6 | 5 | 5.2 |
| 6 | G021846 | | mRNA | 0.3 | 112.1 | 44.8 | 5 | 12.6 |
| 7 | | | B | 1.0 | 14.6 | 4.1 | 5 | 1.6 |
| 8 | G023420 | | | 0.3 | 157.3 | 86.7 | 5 | 17.6 |
| 9 | | | | 1.0 | 28.7 | 5.9 | 5 | 3.2 |

### Example 7—In Vivo Editing with Orthogonal Cas9-Cas9 Fusion Constructs in Mice

Two types of LNPs were formulated as described in Example 1, with a molar ratio of 50% Lipid A, 38% cholesterol, 9% DSPC, and 3% PEG2k-DMG. LNP1 designates LNPs that comprised a Nme2 sgRNA (SEQ ID NO: 252), a Spy sgRNA (SEQ ID NO: 201), or both a Nme2 sgRNA and a Spy sgRNA (SEQ ID NOs: 252 and 201) at a ratio of 1:2 Nme2 sgRNA:Spy sgRNA by weight. The sgRNA used in this experiment targeted the mouse TTR gene start codon. LNP2 designates a second type of LNPs that comprised only mRNA. Transport and storage solution (TSS) used in LNP preparation was dosed in the experiment as a vehicle-only negative control.

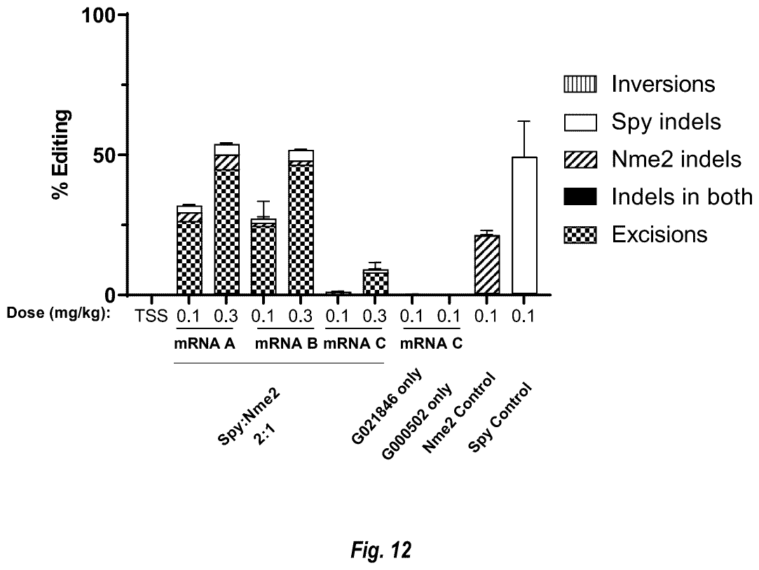

Editing efficiency was tested in vivo using LNP1 and LNP2 species as described above. CD-1 female mice (N=5 animals in each group) about 6 weeks of age were used in the experiment and were weighed pre-dose. Animals received a lateral tail vein injection at a dose of 0.1 milligrams per kilogram body weight (e.g., 0.1 mg/kg, or 0.1 mpk) or 0.3 mg/kg of a LNP mixture comprising a ratio of 2:1 LNP1:LNP2 by volume, unless otherwise specified. The animals were observed at approximately 24 hours post dose for adverse effects. Animals were euthanized 6 days post dose by cardiac exsanguination under isoflurane anesthesia and cervical dislocation. Blood was collected via cardiac puncture into serum separator tubes or into tubes containing buffered sodium citrate for plasma. Liver tissue was collected from the left lateral lobe from each animal for DNA extraction and NGS analysis as described in Example 1. Editing percentages from the mRNA sequences described in Table 19 are shown in Table 20 and illustrated in FIG. 12.

TABLE 19
mRNA sequences

| | SEQ | SpyCas9 | | NmeCas9 | Relative orientation |
|---|---|---|---|---|---|
| Construct | ID NO. | moiety | Linker | moiety | of nuclease moieties |
| mRNA A | 1 | R1333K | linker | WT Nme2 | R1333K Spy-linker 81aa- |
| | | Spy | 81aa | | WT Nme2-SV40NLS |
| mRNA B | 8 | R1333K | linker | WT Nme2 | SV40NLS-WT Nme2-linker |
| | | Spy | 41aa | | 41aa-R1333K Spy- |
| | | | | | SV40NLS-HiBit |
| mRNA C | 11 | R1333K | linker | WT Nme2 | WT Nme2-linker 41aa- |
| | | Spy | 41aa | | R1333K Spy-SV40NLS-HiBit |

TABLE 20
Editing percentages in liver tissue (N = 5 for all groups).

| Guide | | | Dose | % Guide1 | | % Guide | | % | | % indels | | % | |
|---|---|---|---|---|---|---|---|---|---|---|---|---|---|
| RNA | mRNA | gRNA:mRNA | (mg/kg) | (Spy) | SD | 2 (Nme2) | SD | Excisions | SD | in both | SD | Inversion | SD |
| TSS | − | − | − | 0.01 | 0.0 | 0.0 | 0.0 | 0.1 | 0.1 | 0.0 | 0.0 | 0.0 | 0.0 |
| G021846/ | mRNA | 2:1 | 0.1 | 2.4 | 0.5 | 3.2 | 0.7 | 26.1 | 2.3 | 0.1 | 0.1 | 0.1 | 0.2 |
| G000502 | A | | 0.3 | 3.8 | 0.6 | 5.1 | 0.7 | 44.6 | 2.7 | 0.2 | 0.3 | 0.1 | 0.3 |
| | mRNA | | 0.1 | 1.8 | 0.8 | 0.6 | 0.2 | 24.8 | 8.7 | 0.0 | 0.0 | 0.0 | 0.0 |
| | B | | 0.3 | 4.0 | 0.4 | 1.3 | 0.2 | 46.2 | 2.1 | 0.1 | 0.1 | 0.1 | 0.1 |
| | mRNA | | 0.1 | 0.1 | 0.0 | 0.1 | 0.1 | 0.9 | 0.4 | 0.0 | 0.0 | 0.0 | 0.0 |
| | C | | 0.3 | 0.9 | 0.3 | 0.3 | 0.2 | 7.9 | 3.8 | 0.1 | 0.1 | 0.0 | 0.0 |
| G021846 | mRNA | | 0.1 | 0.0 | 0.0 | 0.3 | 0.1 | 0.0 | 0.0 | 0.0 | 0.0 | 0.0 | 0.0 |
| | C | | | | | | | | | | | | |
| G0000502 | mRNA | | 0.1 | 0.1 | 0.1 | 0.0 | 0.0 | 0.0 | 0.0 | 0.0 | 0.0 | 0.0 | 0.0 |
| | C | | | | | | | | | | | | |
| G021846 | Nme2 | | 0.1 | 0.1 | 0.1 | 21.2 | 1.7 | 0.0 | 0.0 | 0.2 | 0.1 | 0.0 | 0.0 |
| | control | | | | | | | | | | | | |
| G000502 | Spy | 1:2 | 0.1 | 49.4 | 12.6 | 0.0 | 0.0 | 0.0 | 0.0 | 0.0 | 0.0 | 0.0 | 0.0 |
| | Control | | | | | | | | | | | | |

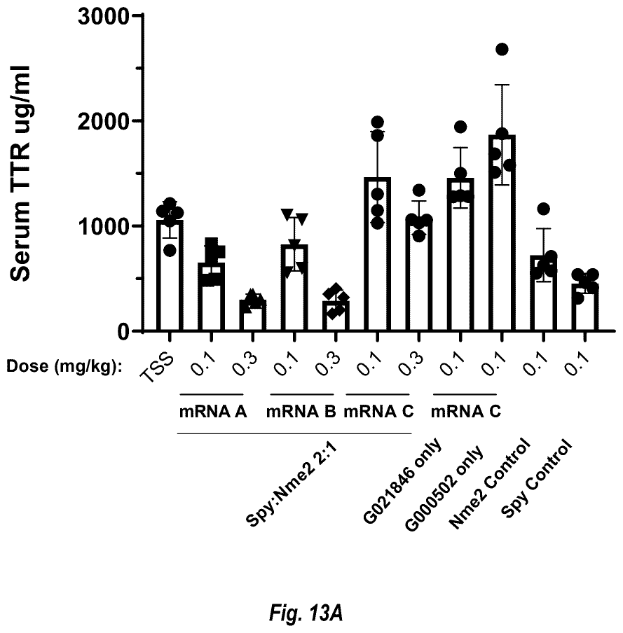

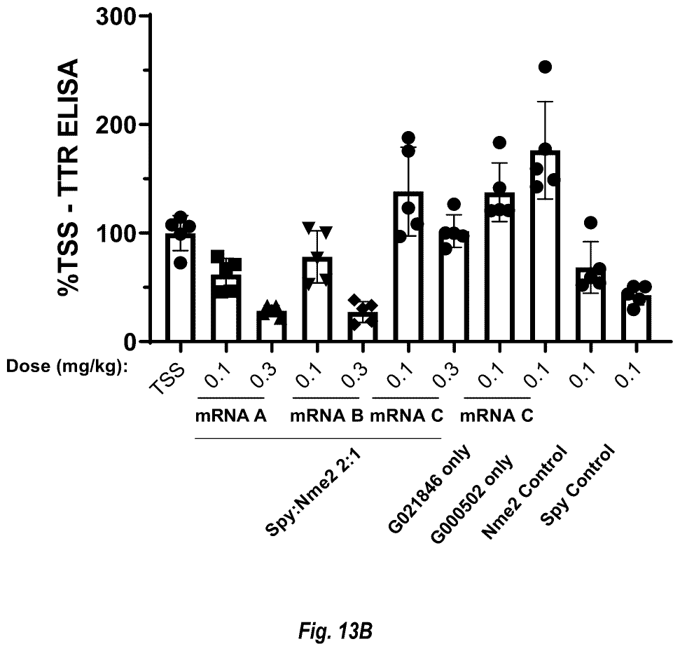

The total serum TTR levels were determined using a Prealbumin (TTR) ELISA Kit, Mouse (Aviva Systems; cat #OKIA00111) (N=5 animals in each group). Kit reagents and standards were prepared according to the manufacturer's protocol. Mouse serum was diluted between 10000 to 50000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 or Clariostar plate reader at an absorbance of 450 nm. Serum TTR levels were calculated by SoftMax Pro software ver. 6.4.2 or Mars software ver. 3.31 using a four-parameter logistic curve fit off the standard curve. Final serum values were adjusted for the assay dilution. Serum TTR levels compared to the control group, which consisted of animals sham-treated with vehicle (TSS), are shown in Table 21 and illustrated in FIGS. 13A and 13B.

TABLE 21
Serum TTR analysis.

| | | | | Serum | | | |
|---|---|---|---|---|---|---|---|
| | | | Dose | TTR | | | |
| Guide RNA | mRNA | gRNA:mRNA | (mg/kg) | ug/ml | SD | N | % TSS |
| TSS | − | − | − | 1059.5 | 172.1 | 5 | 100 |
| G021846/ | mRNA | 2:1 | 0.1 | 654.6 | 157.3 | 5 | 61.8 |
| G000502 | A | | 0.3 | 301.3 | 51.2 | 5 | 28.4 |
| | mRNA | | 0.1 | 827.4 | 253.6 | 5 | 78.1 |
| | B | | 0.3 | 289.7 | 102.1 | 5 | 27.3 |
| | mRNA | | 0.1 | 1466.0 | 433.1 | 5 | 138.4 |
| | C | | 0.3 | 1079.8 | 158.7 | 5 | 101.9 |
| G021846 | mRNA | | 0.1 | 1458.4 | 286.9 | 5 | 137.6 |
| | C | | | | | | |
| G000502 | mRNA | | 0.1 | 1867.9 | 476.0 | 5 | 176.3 |
| | C | | | | | | |
| G021846 | Nme2 | | 0.1 | 724.8 | 252.5 | 5 | 68.4 |
| | control | | | | | | |
| G000502 | Spy | 1:2 | 0.1 | 453.8 | 93.8 | 5 | 42.8 |
| | Control | | | | | | |

### Example 8—Editing and Expression of Full-Length Intein-Mediated Split Orthogonal Cas9-Cas9 Fusion mRNA

Intein-mediated split orthogonal Cas9-Cas9 fusion mRNA was tested to measure editing in transfected cells. Primary mouse hepatocytes, PMH (Gibco, MC882), were thawed and resuspended in 50 mL Cryopreserved Hepatocyte Recovery Media (CHRM) (Invitrogen, CM7000) followed by centrifugation. Cells were resuspended in hepatocyte medium with plating supplements: William's E Medium Plating Supplements with FBS content (Gibco, Cat. A13450). Cells were pelleted by centrifugation, resuspended in media and plated at a density of 15,000 cells/well on Bio-coat collagen I coated 96-well plates (Corning #354407). Plated cells were allowed to settle overnight in a tissue culture incubator at 37° C. and 5% CO₂ atmosphere. The next morning, cells were checked for monolayer formation, existing media was aspirated and cells were plated with 100 uL Cellartis Power Primary HEP Medium (Takara Bio, Cat no. Y20020) prior to transfection.

Lipofection of cells with mRNA and gRNAs used pre-mixed lipid formulations as described in Example 1. The lipofection reagent contained ionizable Lipid A, cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA (e.g., Cas9 mRNA and gRNA) at a lipid amine to RNA phosphate (N:P) molar ratio of about 6 to produce lipid nucleic acid assemblies.

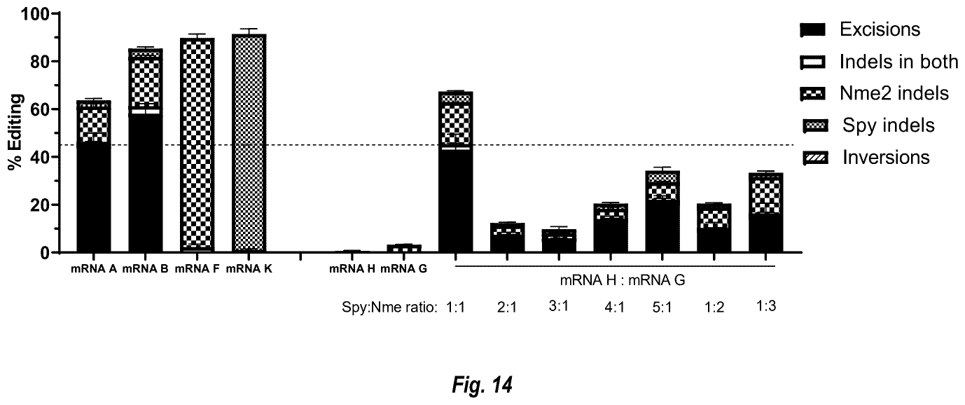

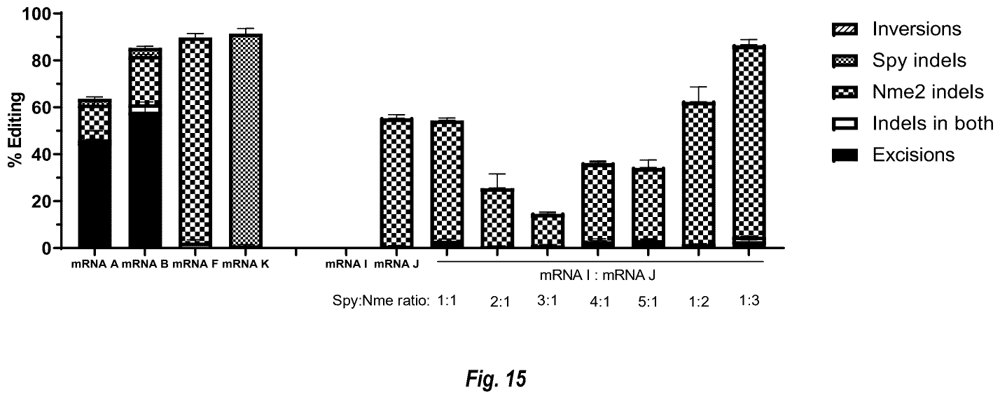

Orthogonal Cas9-Cas9 fusion mRNAs are described in Table 22. Lipofections were performed using, in each well, 180 ng orthogonal Cas9-Cas9 fusion mRNA A equivalents of a mRNA construct as described in Table 23, 25 nM SpyCas9 guide (SEQ ID NO: 201) and 25 nM NmeCas9 guide (SEQ ID NO: 250). Different ratios of Spy Intein mRNA to Nme2 Intein mRNA for orthogonal Cas9-Cas9 fusion were tested as follows: 1:1, 2:1, 3:1, 4:1, 5:1, 1:2, and 1:3 for a total of 180 ng total equivalents of orthogonal Cas9-Cas9 fusion mRNA A. Briefly, cells were incubated at 37° C., 5% CO2 for 24 hours prior to treatment with the lipid nucleic acid mixtures. Lipid nucleic acid mixtures were incubated in media containing 10% fetal bovine serum (FBS) at 37° C. for 5 minutes. Post-incubation, the lipid nucleic acid mixtures were added to the cells as indicated above. The cells were lysed 5 days post-treatment and NGS analysis was performed as described in Example 1. Table 24 and FIG. 14 show editing in cells transfected with Spy intein mRNA H and Nme2 intein mRNA G. Table 25 and FIG. 15 show editing in cells transfected with Spy intein mRNA I and Nme2 intein mRNA J.

TABLE 22
mRNA sequences

| | SEQ | SpyCas9 | | NmeCas9 | Relative orientation |
|---|---|---|---|---|---|
| Construct | ID NO. | moiety | Linker | moiety | of nuclease moieties |
| mRNA A | 1 | R1333K | linker | WT | R1333K Spy-linker 81aa- |
| | | Spy | 81aa | Nme2 | WT Nme2-SV40NLS |
| | | | SEQ ID | | |
| | | | NO: 150 | | |
| mRNA B | 8 | R1333K | linker | WT | SV40NLS-WT Nme2-linker |
| | | Spy | 41aa | Nme2 | 41aa-R1333K Spy- |
| | | | SEQ ID | | SV40NLS-HiBit |
| | | | NO: 154 | | |
| mRNA F | 20 | none | none | WT | WT Nme2 |
| (Nme2Cas9) | | | | Nme2 | |
| mRNA G | 23 | none | linker | WT | SV40NLS-Nucleoplasmin- |
| (Nme2Cas9 | | | 81aa | Nme2 | WT Nme2-linker 81 aa - |
| intein) | | | SEQ ID | | NpuN |
| | | | NO: 158 | | |
| mRNA H | 16 | R1333K | none | none | NpuC-R1333K Spy- |
| (SpyCas9 | | Spy | | | SV40NLS |
| intein) | | | | | |
| mRNA I | 19 | R1333K | none | none | SV40NLS-R1333K Spy- |
| (SpyCas9 | | Spy | | | NpuN |
| intein) | | | | | |
| mRNA J | 22 | none | linker | WT | NpuC- linker 81 aa - |
| (Nme2Cas9 | | | 81aa | Nme2 | WT Nme2-SV40NLS |
| intein) | | | SEQ ID | | |
| | | | NO: 158 | | |
| mRNA K | 25 | WT Spy | none | none | WT Spy-HiBit |
| (SpyCas9) | | | | | |

TABLE 23
Transfected mRNA

| mRNA | size relative to mRNA A | ng/well |
|---|---|---|
| mRNA A | 1 | 180.00 |
| mRNA B | 1.00 | 179.22 |
| mRNA F | 0.43 | 77.98 |
| mRNA G | 0.47 | 84.68 |
| mRNA H | 0.56 | 100.26 |
| mRNA I | 0.58 | 105.26 |
| mRNA J | 0.46 | 83.55 |
| mRNA K | 0.58 | 104.23 |

TABLE 24
Editing in transfected cells (n = 3 for all groups)

| mRNA | Spy indels | | Nme2 indels | | Excisions | | Indels in both | | Inversions | |
|---|---|---|---|---|---|---|---|---|---|---|
| condition | Mean | SD | Mean | SD | Mean | SD | Mean | SD | Mean | SD |
| mRNA A | 2.55 | 0.77 | 14.88 | 1.16 | 45.09 | 4.86 | 1.17 | 0.21 | 0.01 | 0.01 |
| mRNA B | 3.54 | 0.62 | 20.60 | 0.70 | 58.10 | 2.58 | 3.17 | 0.98 | 0.00 | 0.00 |
| mRNA F | 0.01 | 0.01 | 87.50 | 1.67 | 0.02 | 0.03 | 2.26 | 1.22 | 0.00 | 0.00 |
| mRNA K | 90.31 | 2.19 | 0.01 | 0.01 | 0.03 | 0.03 | 1.10 | 0.39 | 0.00 | 0.00 |
| mRNA H | 0.68 | 0.17 | 0.01 | 0.01 | 0.00 | 0.00 | 0.00 | 0.00 | 0.00 | 0.00 |
| mRNA G | 0.02 | 0.02 | 3.24 | 0.19 | 0.00 | 0.00 | 0.01 | 0.01 | 0.00 | 0.00 |
| 1:1 mRNA | 4.40 | 0.32 | 17.51 | 3.06 | 43.04 | 6.49 | 2.42 | 0.38 | 0.01 | 0.01 |
| H:mRNA G | | | | | | | | | | |
| 2:1 mRNA | 1.05 | 0.37 | 3.84 | 0.24 | 7.17 | 1.28 | 0.32 | 0.05 | 0.00 | 0.00 |
| H:mRNA G | | | | | | | | | | |
| 3:1 mRNA | 1.67 | 1.06 | 2.20 | 0.16 | 5.84 | 1.39 | 0.11 | 0.09 | 0.00 | 0.00 |
| H:mRNA G | | | | | | | | | | |
| 4:1 mRNA | 2.24 | 0.42 | 4.22 | 0.63 | 13.92 | 1.31 | 0.16 | 0.24 | 0.00 | 0.01 |
| H:mRNA G | | | | | | | | | | |
| 5:1 mRNA | 4.89 | 1.42 | 7.34 | 0.44 | 21.20 | 2.62 | 0.83 | 0.69 | 0.00 | 0.00 |
| H:mRNA G | | | | | | | | | | |
| 1:2 mRNA | 1.58 | 0.30 | 8.63 | 1.30 | 9.98 | 2.64 | 0.35 | 0.06 | 0.00 | 0.00 |
| H:mRNA G | | | | | | | | | | |
| 1:3 mRNA | 1.70 | 0.70 | 15.27 | 2.59 | 15.31 | 5.33 | 1.06 | 0.54 | 0.00 | 0.00 |
| H:mRNA G | | | | | | | | | | |

TABLE 25
Editing in transfected cells (n = 3 for all groups)

| mRNA | Spy indels | | Nme2 indels | | Excisions | | Indels in both | | Inversions | |
|---|---|---|---|---|---|---|---|---|---|---|
| condition | Mean | SD | Mean | SD | Mean | SD | Mean | SD | Mean | SD |
| mRNA A | 2.55 | 0.77 | 14.88 | 1.16 | 45.09 | 4.86 | 1.17 | 0.21 | 0.01 | 0.01 |
| mRNA B | 3.54 | 0.62 | 20.60 | 0.70 | 58.10 | 2.58 | 3.17 | 0.98 | 0.00 | 0.00 |
| mRNA F | 0.01 | 0.01 | 87.50 | 1.67 | 0.02 | 0.03 | 2.26 | 1.22 | 0.00 | 0.00 |
| mRNA K | 90.31 | 2.19 | 0.01 | 0.01 | 0.03 | 0.03 | 1.10 | 0.39 | 0.00 | 0.00 |
| mRNA I | 0.03 | 0.05 | 0.00 | 0.01 | 0.00 | 0.00 | 0.00 | 0.00 | 0.00 | 0.00 |
| mRNA J | 0.02 | 0.02 | 54.16 | 1.65 | 0.00 | 0.00 | 1.08 | 0.08 | 0.01 | 0.02 |
| 1:1 mRNA | 0.05 | 0.07 | 51.32 | 1.11 | 1.75 | 0.27 | 1.27 | 0.86 | 0.00 | 0.00 |
| I:mRNA J | | | | | | | | | | |
| 2:1 mRNA | 0.06 | 0.05 | 24.64 | 6.08 | 0.56 | 0.26 | 0.32 | 0.11 | 0.00 | 0.00 |
| I:mRNA J | | | | | | | | | | |
| 3:1 mRNA | 0.07 | 0.05 | 13.28 | 0.86 | 1.00 | 0.39 | 0.24 | 0.24 | 0.00 | 0.00 |
| I:mRNA J | | | | | | | | | | |
| 4:1 mRNA | 0.32 | 0.36 | 33.04 | 1.17 | 2.65 | 1.28 | 0.26 | 0.26 | 0.00 | 0.00 |
| I:mRNA J | | | | | | | | | | |
| 5:1 mRNA | 0.21 | 0.12 | 30.63 | 3.49 | 2.87 | 1.08 | 0.52 | 0.33 | 0.00 | 0.00 |
| I:mRNA J | | | | | | | | | | |
| 1:2 mRNA | 0.03 | 0.05 | 60.63 | 6.39 | 0.74 | 0.07 | 0.95 | 0.22 | 0.00 | 0.00 |
| I:mRNA J | | | | | | | | | | |
| 1:3 mRNA | 0.05 | 0.06 | 81.30 | 2.68 | 2.97 | 0.59 | 1.95 | 0.12 | 0.16 | 0.27 |
| I:mRNA J | | | | | | | | | | |

In addition, intein-mediated split orthogonal Cas9-Cas9 fusion mRNA was tested to measure the expression of full-length orthogonal Cas9-Cas9 fusion protein in transfected cells. Primary mouse hepatocytes, PMH (Gibco, MC883), were thawed and resuspended in 50 mL Cryopreserved Hepatocyte Recovery Media (CHRM) (Invitrogen, CM7000) followed by centrifugation. Cells were resuspended in hepatocyte medium with plating supplements: William's E Medium Plating Supplements with FBS content (Gibco, Cat. A13450). Cells were pelleted by centrifugation, resuspended in media and plated at a density of 350,000 cells/well on Bio-coat collagen I coated 6-well plates (Corning #356400). Plated cells were agitated by shaking the plate by hand every 15 minutes for 1 hour to ensure even distribution and then were allowed to settle and adhere overnight in a tissue culture incubator at 37° C. and 5% CO₂ atmosphere. After incubation cells were checked for monolayer formation, existing media was aspirated and cells were plated with 2 mL hepatocyte maintenance medium: William's E Medium (Gibco, Cat. A12176-01) plus supplement pack (Gibco, Cat. CM3000). Cells were transfected later that day according to the manufacturer's protocol with 7.5 uL Lipofectamine Messenger Max (ThermoFisher, Cat. LMRNA001) diluted in OptiMem Medium (ThermoFisher, Cat. 31985062). Each well contained 4640 ng orthogonal Cas9-Cas9 fusion mRNA A equivalents of a mRNA construct, as described in Table 26.

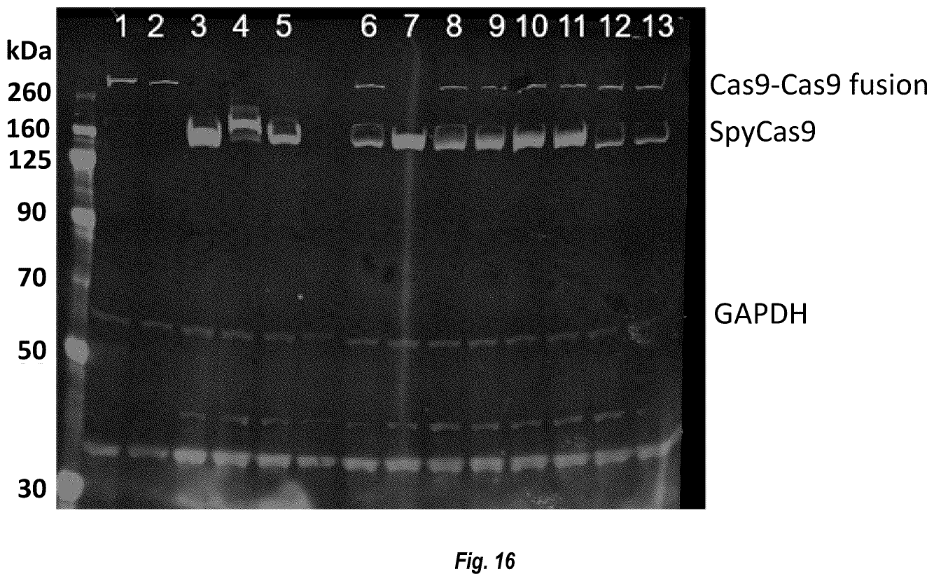

Different ratios of Spy Intein mRNA H to Nme2 Intein mRNA G were tested as follows: 1:1, 2:1, 3:1, 4:1, 5:1, 1:2, and 1:3 for a total of 4640 ng total equivalents of orthogonal Cas9-Cas9 fusion mRNA A. Protein isolation and western blot analysis were performed as follows. 24 hours post-treatment, cell plates were placed on ice and washed once with cold PBS, and then 50 uL/well of RIPA Lysis Buffer (Boston Bioproducts, Cat. BP-115) supplemented with 1× Protease Inhibitor (ThermoFisher, Cat. 1861278), 0.01M DTT (Cat. P2325), and 1× Benzonase Nuclease (EMD Millipore, Cat. 71206-25KUN). Cells were scraped, lysates were pipetted up and down and left to incubate five minutes on ice. Lysates were moved to an Eppendorf tube and spun down in a tabletop centrifuge at 4° C. for 15 minutes at 13,000×g. Supernatant was collected in a fresh Eppendorf tube. 8 uL/lane of each protein sample was run in a Bolt 8% Bis-Tris Plus gel (ThermoFisher, Cat. NW00085BOX) with 20×MOPS Running Buffer (ThermoFisher, Cat. NP0001). Samples were run on the gel for 1 hour at 150 volts with Chameleon Duo Pre-stained Protein Ladder (Licor, Cat. 928-60000) and transferred to a 0.45 m Nitrocellulose Membrane (ThermoFisher, Cat. 77010). The membrane was blocked in LiCor Odyssey Blocking Buffer (TBS) (LiCor, Cat. 927-60001) for 1 hour and incubated overnight at 4° C. with antibodies against SpyCas9 (Daigenode, Cat. 15310258) and GAPDH (GeneTex, Cat. GTX627408-01) diluted in 1:1 TB ST and LiCor Blocking Buffer. The next morning, blots were washed in X TBST and incubated for 1 hour at room temperature with goat anti-rabbit IgG (LiCor, Cat. 926-32211) and goat anti-mouse (LiCor, Cat. 926-68070) diluted in 1:1 LiCor Blocking Buffer and TBST and imaged on the LiCor Odyssey Imager (FIG. 16). Table 27 shows the percentage orthogonal Cas9-Cas9 fusion protein expression of total Spy protein expression detected in the Western blot.

TABLE 26
Transfected mRNA

| mRNA | Size relative to 1265 | Total ng/well |
|---|---|---|
| mRNA A | 1 | 4640.0 |
| mRNA B | 1.00 | 4620.0 |
| mRNA F | 0.43 | 2010.1 |
| mRNA G | 0.47 | Ratio dependent |
| mRNA H | 0.56 | Ratio dependent |
| mRNA I | 0.58 | Ratio dependent |
| mRNA J | 0.46 | Ratio dependent |
| mRNA K | 0.58 | 2686.8 |

TABLE 27
orthogonal Cas9-Cas9 fusion protein expression (%) of
total Spy protein expression detected in Western blot

| | | Orthogonal Cas9-Cas9 fusion |
|---|---|---|
| Sample | mRNA condition | (% of total Spy signal) |
| 1 | mRNA A orthogonal Cas9-Cas9 | 96 |
| | fusion | |
| 2 | mRNA B orthogonal Cas9-Cas9 | 96 |
| | fusion | |
| 3 | mRNA H SpyCas9 Intein | 1 |
| 4 | mRNA I SpyCas9 Intein | 4 |
| 5 | mRNA K SpyCas9 | 5 |
| 6 | 1:1 mRNA H:mRNA G | 43 |
| 7 | 1:1 mRNA H:mRNA F | 4 |
| 8 | 2:1 mRNA H:mRNA G | 31 |
| 9 | 3:1 mRNA H:mRNA G | 24 |
| 10 | 4:1 mRNA H:mRNA G | 27 |
| 11 | 5:1 mRNA H:mRNA G | 26 |
| 12 | 1:2 mRNA H:mRNA G | 51 |
| 13 | 1:3 mRNA H:mRNA G | 49 |

### Example 9—Excision with Exemplary Spy Cas9 and Nme Cas9 Guide Designs

Additional guide scaffolds were tested for excision efficacy in primary mouse hepatocytes (PMH) at the PCSK9 locus. Primary mouse hepatocytes, PMH (Gibco, MCM855), were thawed and resuspended in Cryopreserved Hepatocyte Recovery Media (CHRM) (Invitrogen, CM7000) followed by centrifugation. Cells were resuspended in hepatocyte medium with plating supplements: William's E Medium Plating Supplements with FBS content (Gibco, Cat. A13450). Cells were pelleted by centrifugation, resuspended in media and plated at a density of 15,000 cells/well on Bio-coat collagen I coated 96-well plates (Corning #354407). Plated cells were allowed to settle and adhere for 4-6 hours in a tissue culture incubator at 37° C. and 5% CO₂ atmosphere. After incubation cells were checked for monolayer formation, existing media was aspirated and cells were plated with 100 uL hepatocyte maintenance medium: William's E Medium (Gibco, Cat. A12176-01) plus supplement pack (Gibco, Cat. CM3000) and left to adhere overnight prior to transfection. Lipofection of cells with mRNA and gRNAs used pre-mixed lipid formulations as described in Example 1. The lipofection reagent contained ionizable Lipid A, cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA (e.g., Cas9 mRNA and gRNA) at a lipid amine to RNA phosphate (N:P) molar ratio of about 6 to produce lipid nucleic acid assemblies.

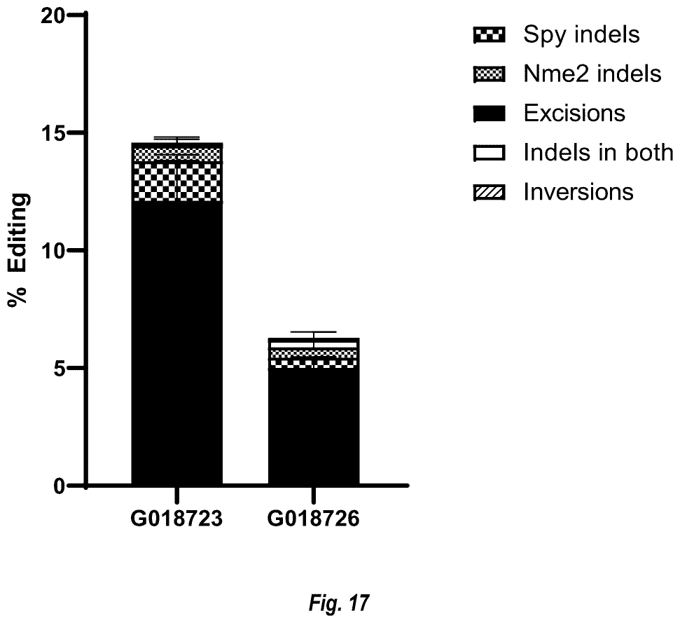

Lipofections were performed using orthogonal Cas9-Cas9 fusion mRNA A (SEQ ID NO: 1), Nme guide G020362, and either Spy guide G018723 or G018726. Equivalent amounts of Nme and Spy gRNAs were used at total ratio of gRNA to mRNA of 1:2 by weight. Briefly, cells were incubated at 37° C., 5% CO2 overnight prior to treatment with the lipid nucleic acid mixtures. Lipid nucleic acid mixtures were incubated in media containing 10% fetal bovine serum (FBS) at 37° C. for 5 minutes. Post-incubation, the lipid nucleic acid mixtures were added to the cells at 723 ng Cas9 mRNA (100 nM of each gRNA). The cells were lysed 5 days post-treatment and NGS analysis was performed as described in Example 1. Table 28 and FIG. 17 show editing data in PMH.

TABLE 28
Mean percent editing in PMH

| | Spy indels | | Nme2 indels | | Excisions | | Indels in both | | Inversions | |
|---|---|---|---|---|---|---|---|---|---|---|
| Sample | Mean | SD | Mean | SD | Mean | SD | Mean | SD | Mean | SD |
| G018723 | 1.69 | 0.28 | 0.66 | 0.36 | 12.10 | 1.71 | 0.12 | 0.14 | 0.00 | 0.00 |
| G018726 | 0.43 | 0.04 | 0.44 | 0.26 | 5.00 | 0.43 | 0.33 | 0.33 | 0.07 | 0.00 |

### Example 10—Excision with Exemplary Spy Cas9 and Nme Cas9 Guide Designs

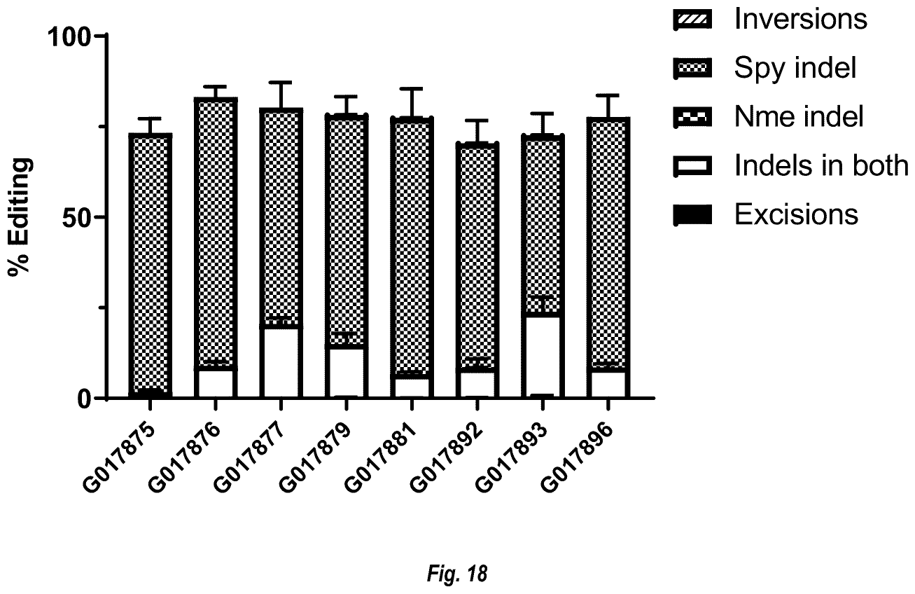

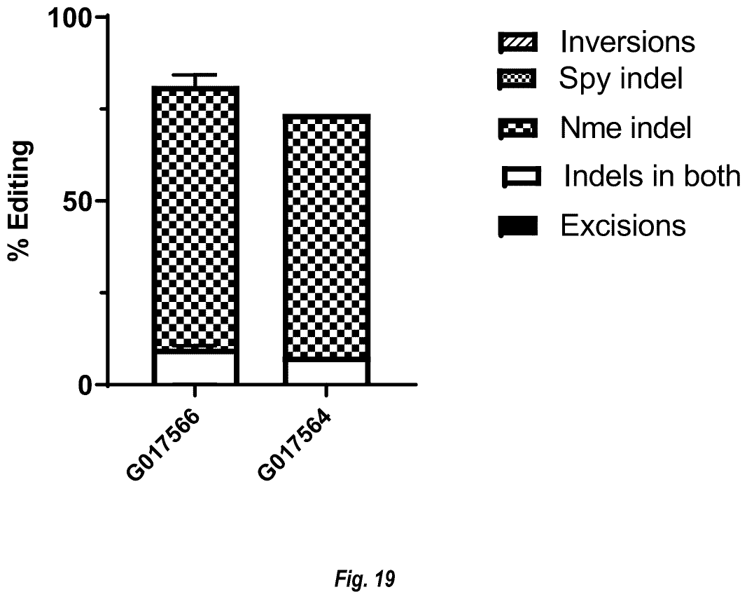

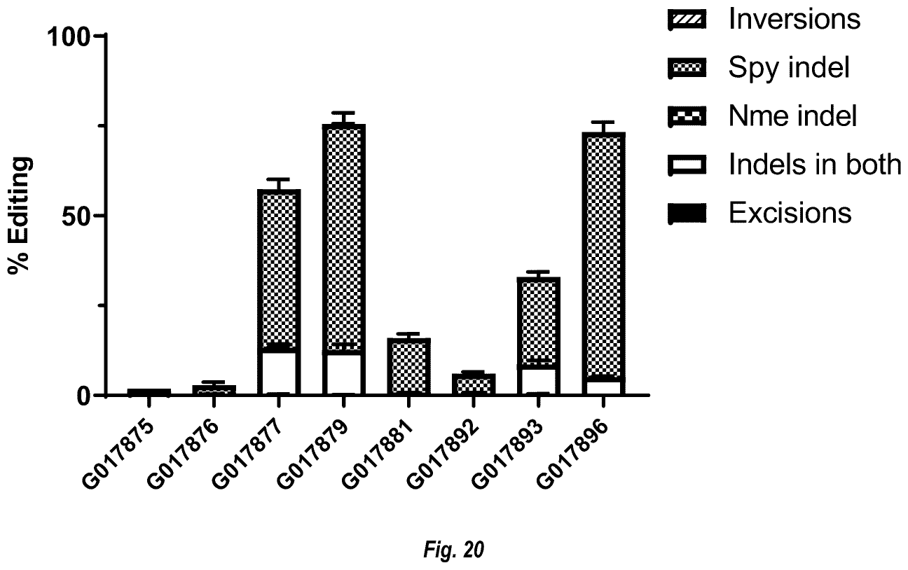

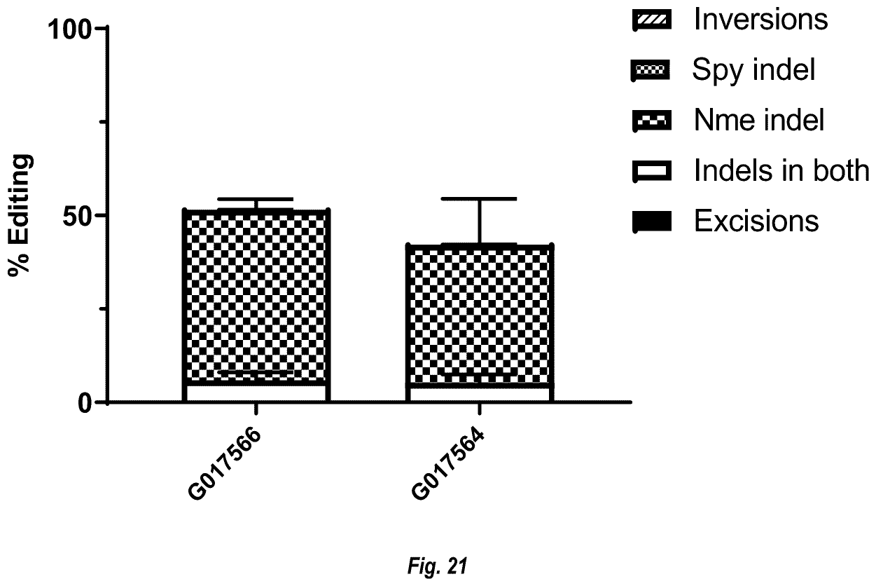

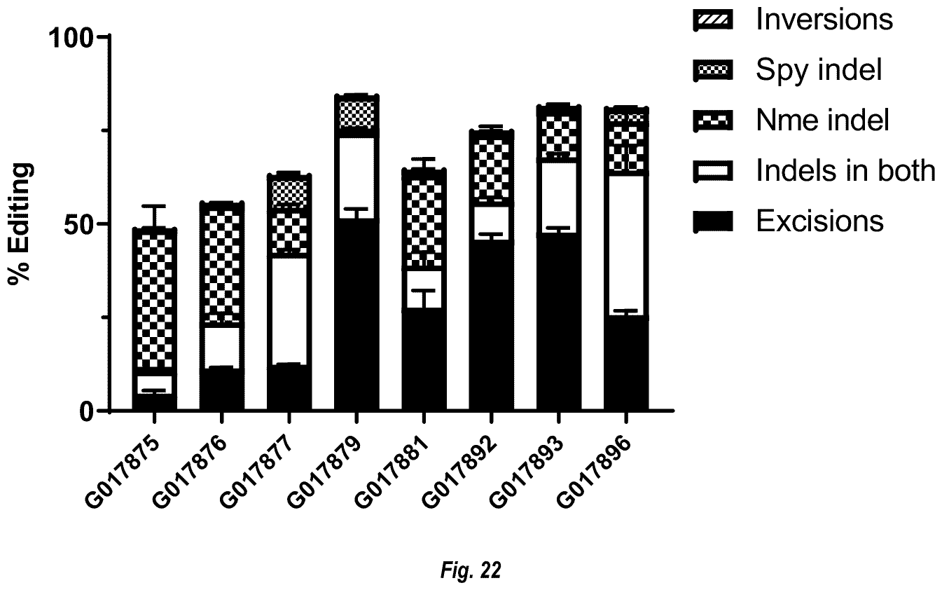

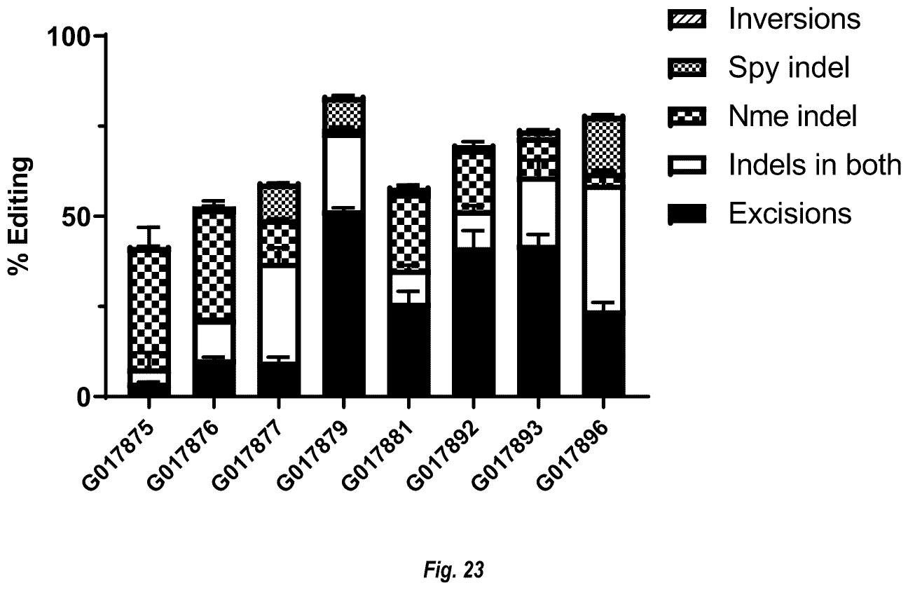

Additional guide scaffolds were tested for excision efficacy in Hepa1-6 cells. Cells were cultured in DMEM media supplemented with 10% fetal bovine serum at a density of 15,000 cells/well in a 96-well plate and left to adhere overnight prior to transfection. Cells were transfected according to the manufacturer's protocol with 0.6 uL/well Lipofectamine Messenger Max (ThermoFisher, Cat. LMRNA001) diluted in OptiMem Medium (ThermoFisher, Cat. 31985062). Each well contained 100 ng SpyCas9 mRNA equivalents of orthogonal Cas9-Cas9 fusion or Nme2Cas9 mRNA as described in Table 29, 50 nM SpyCas9 guide and 50 nM NmeCas9 guide. For Cas9 controls, 50 nM of only the species appropriate guide was used. DNA isolation and NGS analysis were performed as described in Example 1. Table 30 shows percent editing at the PCSK9 locus by these guides. FIG. 18 shows percent editing at the PCSK9 locus by Spy guides and SpyCas9. FIG. 19 shows percent editing at the PSCK9 locus by Nme guides and Nme2Cas9. FIG. 20 shows percent editing at the PSCK9 locus by Spy guides and orthogonal Cas9-Cas9 fusion. FIG. 21 shows percent editing at the PSCK9 locus by Nme guides and orthogonal Cas9-Cas9 fusion. FIG. 22 shows percent editing at the PSCK9 locus by Spy guides and Nme guide G017566 and orthogonal Cas9-Cas9 fusion. FIG. 23 shows percent editing at the PSCK9 locus by Spy guides and Nme guide G017564 and orthogonal Cas9-Cas9 fusion.

TABLE 29
Nuclease mRNA amounts used in PMH transfection

| mRNA Alias | size relative to Spy | total ng/well |
|---|---|---|
| mRNA A | 1.86 | 186 |
| mRNA F (Nme2 Cas9) | 0.88 | 88 |
| mRNA X (Spy Cas9) | 1 | 100 |

TABLE 30
Percent editing at the PCSK9 locus by these guides (n = 2 for all groups)

| | Spy | Nme2 | Spy indel | | Nme indel | | Excisions | | Indels in both | | Inversions | |
|---|---|---|---|---|---|---|---|---|---|---|---|---|
| mRNA | guide | guide | Mean | SD | Mean | SD | Mean | SD | Mean | SD | Mean | SD |
| SpyCas9 | G017875 | none | 71.52 | 3.92 | 0.28 | 0.35 | 0.02 | 0.03 | 1.45 | 0.85 | 0.00 | 0.00 |
| mRNA X | G017876 | | 74.30 | 2.79 | 0.01 | 0.02 | 0.05 | 0.00 | 8.88 | 1.18 | 0.00 | 0.00 |
| | G017877 | | 59.76 | 6.87 | 0.01 | 0.01 | 0.36 | 0.01 | 20.20 | 1.68 | 0.00 | 0.00 |
| | G017879 | | 63.28 | 5.08 | 0.01 | 0.00 | 0.21 | 0.08 | 14.73 | 2.97 | 0.04 | 0.04 |
| | G017881 | | 70.68 | 8.10 | 0.03 | 0.00 | 0.08 | 0.01 | 6.59 | 0.61 | 0.05 | 0.05 |
| | G017892 | | 61.97 | 6.32 | 0.01 | 0.02 | 0.15 | 0.07 | 8.27 | 2.46 | 0.01 | 0.02 |
| | G017893 | | 48.74 | 6.07 | 0.01 | 0.00 | 0.58 | 0.21 | 23.24 | 4.12 | 0.05 | 0.05 |
| | G017896 | | 69.13 | 5.96 | 0.00 | 0.00 | 0.01 | 0.02 | 8.54 | 1.09 | 0.00 | 0.00 |
| Nme2Cas9 | none | G017566 | 0.00 | 0.00 | 71.49 | 2.95 | 0.05 | 0.05 | 9.78 | 0.84 | 0.00 | 0.00 |
| mRNA F | | G017564* | 0.00 | | 66.11 | | 0.01 | | 7.59 | | 0.00 | |
| orthogonal Cas9- | none | G017566 | 0.02 | 0.03 | 45.58 | 2.87 | 0.00 | 0.00 | 5.89 | 2.15 | 0.01 | 0.02 |
| Cas9 fusion mRNA A | | G017564 | 0.02 | 0.02 | 36.90 | 12.35 | 0.00 | 0.00 | 5.23 | 2.25 | 0.03 | 0.05 |
| orthogonal Cas9- | G017875 | none | 1.79 | 0.03 | 0.07 | 0.03 | 0.00 | 0.00 | 0.00 | 0.00 | 0.00 | 0.00 |
| Cas9 fusion mRNA A | G017876 | | 2.56 | 0.86 | 0.06 | 0.05 | 0.04 | 0.05 | 0.20 | 0.07 | 0.00 | 0.00 |
| | G017877 | | 44.11 | 2.72 | 0.08 | 0.10 | 0.32 | 0.09 | 12.87 | 1.07 | 0.01 | 0.01 |
| | G017879 | | 62.92 | 3.06 | 0.01 | 0.00 | 0.13 | 0.06 | 12.48 | 1.63 | 0.02 | 0.02 |
| | G017881 | | 15.14 | 1.17 | 0.01 | 0.01 | 0.01 | 0.01 | 0.76 | 0.06 | 0.00 | 0.00 |
| | G017892 | | 5.42 | 0.50 | 0.01 | 0.02 | 0.01 | 0.01 | 0.61 | 0.18 | 0.01 | 0.01 |
| | G017893 | | 24.31 | 1.44 | 0.02 | 0.00 | 0.37 | 0.10 | 8.22 | 1.24 | 0.05 | 0.02 |
| | G017896 | | 68.15 | 2.79 | 0.00 | 0.01 | 0.01 | 0.02 | 5.10 | 0.27 | 0.00 | 0.00 |
| orthogonal Cas9- | G017875 | G017566 | 0.30 | 0.00 | 37.76 | 6.17 | 4.64 | 0.81 | 6.18 | 0.50 | 0.02 | 0.02 |
| Cas9 fusion mRNA A | G017876 | | 0.65 | 0.05 | 31.27 | 0.74 | 11.40 | 0.19 | 12.30 | 2.41 | 0.07 | 0.03 |
| | G017877 | | 8.69 | 0.78 | 11.98 | 0.94 | 12.39 | 0.03 | 29.93 | 0.82 | 0.11 | 0.06 |
| | G017879 | | 8.61 | 0.23 | 1.13 | 0.02 | 51.50 | 2.52 | 22.95 | 0.72 | 0.26 | 0.12 |
| | G017881 | | 1.34 | 0.20 | 24.29 | 4.28 | 27.60 | 4.64 | 11.24 | 3.58 | 0.11 | 0.02 |
| | G017892 | | 0.83 | 0.05 | 17.91 | 2.15 | 45.77 | 1.53 | 10.31 | 1.07 | 0.19 | 0.00 |
| | G017893 | | 1.20 | 0.13 | 12.40 | 1.86 | 47.73 | 1.26 | 20.04 | 1.05 | 0.24 | 0.08 |
| | G017896 | | 3.43 | 0.15 | 13.16 | 2.46 | 25.63 | 1.14 | 38.67 | 6.62 | 0.34 | 0.04 |
| orthogonal Cas9- | G017875 | G017564 | 0.15 | 0.14 | 33.50 | 5.57 | 3.90 | 0.18 | 3.97 | 4.32 | 0.06 | 0.09 |
| Cas9 fusion mRNA A | G017876 | | 0.72 | 0.08 | 30.61 | 2.25 | 10.41 | 0.57 | 11.01 | 0.03 | 0.07 | 0.02 |
| | G017877 | | 9.88 | 0.21 | 12.00 | 0.18 | 9.74 | 1.20 | 27.42 | 4.13 | 0.10 | 0.03 |
| | G017879 | | 8.77 | 0.54 | 1.01 | 0.11 | 51.72 | 0.65 | 21.52 | 1.59 | 0.11 | 0.05 |
| | G017881 | | 1.45 | 0.13 | 21.19 | 2.28 | 26.04 | 3.18 | 9.18 | 1.25 | 0.08 | 0.04 |
| | G017892 | | 1.04 | 0.01 | 16.86 | 2.22 | 41.48 | 4.55 | 10.17 | 1.29 | 0.24 | 0.03 |
| | G017893 | | 1.92 | 0.05 | 10.90 | 0.86 | 42.13 | 2.82 | 18.87 | 4.51 | 0.09 | 0.13 |
| | G017896 | | 15.74 | 0.36 | 3.27 | 0.32 | 23.95 | 2.19 | 34.86 | 4.04 | 0.13 | 0.07 |

*= only one replicate

### Example 11—Excision with Exemplary Spy Cas9 and Nme Cas9 Guide Designs

Additional guide scaffolds were tested for excision efficacy in primary mouse hepatocytes (PMH) at the TTR locus. Primary mouse hepatocytes, PMH (Gibco, MC855), were thawed and resuspended in 50 mL Cryopreserved Hepatocyte Recovery Media (CHRM) (Invitrogen, CM7000) followed by centrifugation. Cells were resuspended in hepatocyte medium with plating supplements: William's E Medium Plating Supplements with FBS content (Gibco, Cat. A13450). Cells were pelleted by centrifugation, resuspended in media and plated at a density of 15,000 cells/well on Bio-coat collagen I coated 96-well plates (Corning #354407). Plated cells were allowed to settle and adhere for 4-6 hours in a tissue culture incubator at 37° C. and 5% CO₂ atmosphere. After incubation cells were checked for monolayer formation, existing media was aspirated and cells were plated with 100 uL hepatocyte maintenance medium: William's E Medium (Gibco, Cat. A12176-01) plus supplement pack (Gibco, Cat. CM3000) and left to adhere overnight prior to transfection.

Cells were transfected according to the manufacturer's protocol with 0.3 uL/well Lipofectamine Messenger Max (ThermoFisher, Cat. LMRNA001) diluted in OptiMem Medium (ThermoFisher, Cat. 31985062). Orthogonal Cas9-Cas9 fusion mRNA constructs are described in Table 31. Each well contained 100 ng SpyCas9 equivalents of mRNA constructs as described in Table 32, 50 nM Spy Cas9 guide and 50 nM NmeCas9 guide.

TABLE 31
mRNA sequences

| | SEQ | SpyCas9 | | NmeCas9 | Relative orientation |
|---|---|---|---|---|---|
| Construct | ID NO. | moiety | Linker | moiety | of nuclease moieties |
| mRNA A | 1 | R1333K | linker 81aa | WT Nme2 | R1333K Spy-linker 81aa- |
| | | Spy | SEQ ID NO: 150 | | WT Nme2-SV40NLS |
| mRNA L | 38 | R1333K | linker 81aa | WT Nme2 | R1333K Spy-linker 81aa - |
| | | Spy | SEQ ID NO: 158 | | WT Nme2-SV40NLS |

TABLE 32
Nuclease mRNA amounts used in PMH transfection

| mRNA | total ng/well |
|---|---|
| mRNA A | 186 |
| mRNA L | 186 |
| mRNA F (Nme2Cas9) | 88 |

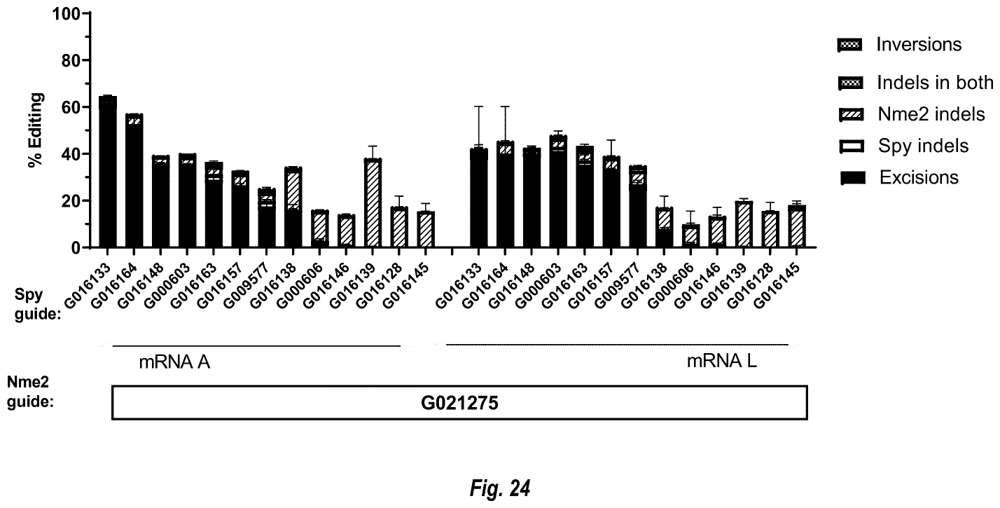

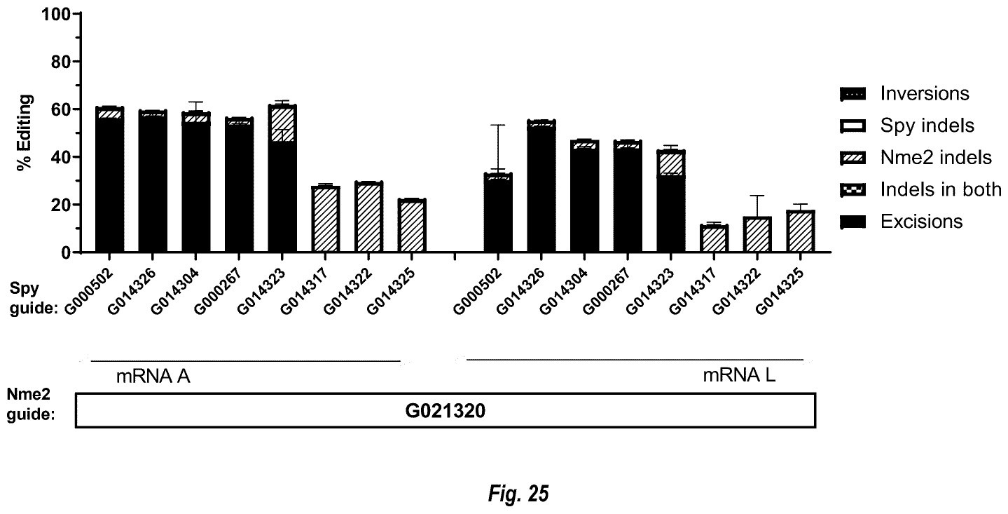

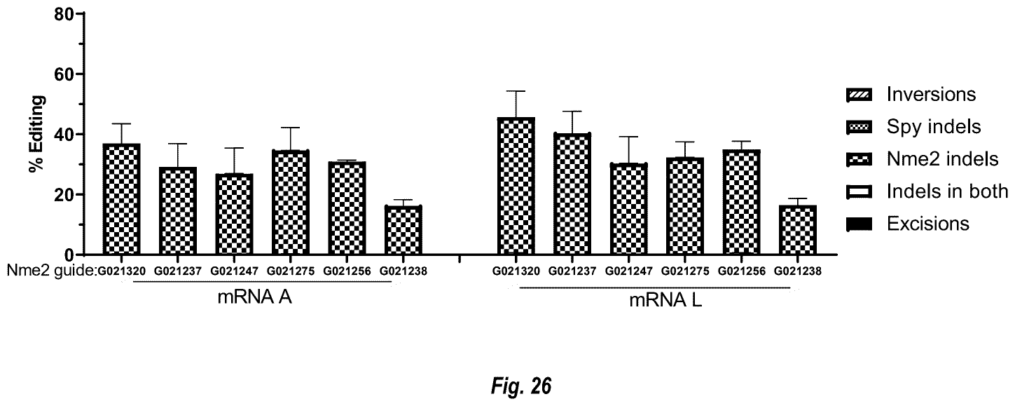

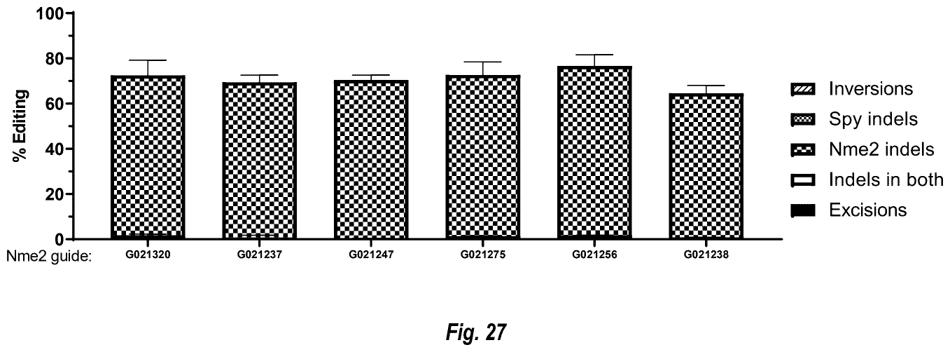

Some cells were transfected with 100 ng SpyCas9 mRNA equivalents of orthogonal Cas9-Cas9 fusion mRNA or Nme mRNA, and only 50 nM NmeCas9 guide RNA. Cells were incubated for 4 days after editing. DNA isolation and NGS analysis were performed as described in Example 1. Percent editing at the TTR locus in transfected cells is indicated in Tables 33 and 34. Percent editing in cells transfected with orthogonal Cas9-Cas9 fusion mRNA, Spy guides and Nme guide G021275 is illustrated in FIG. 24. Percent editing in cells transfected with orthogonal Cas9-Cas9 fusion mRNA, Spy guides and Nme guide G021320 is illustrated in FIG. 25. Percent editing in cells transfected with orthogonal Cas9-Cas9 fusion mRNA and Nme guides is illustrated in FIG. 26. Percent editing in cells transfected with NmeCas9 mRNA and Nme guides is illustrated in FIG. 27.

TABLE 33
Mean percent editing of orthogonal Cas9-Cas9 fusion, Nme guide
and Spy guide at the TTR locus in PMH (n = 2 for all groups)

| Nme | | Spy | Spy Indels | | Nme2 Indels | | Excision | | Indels in both | | Inversions | |
|---|---|---|---|---|---|---|---|---|---|---|---|---|
| gRNA | mRNA | gRNA | Mean | SD | Mean | SD | Mean | SD | Mean | SD | Mean | SD |
| G021275 | mRNA A | G000603 | 1.29 | 0.02 | 2.99 | 0.93 | 34.46 | 1.30 | 0.99 | 0.15 | 0.41 | 0.01 |
| | | G000606 | 0.26 | 0.22 | 12.76 | 0.11 | 2.50 | 1.18 | 0.36 | 0.21 | 0.00 | 0.00 |
| | | G009577 | 2.24 | 0.36 | 3.58 | 0.43 | 17.57 | 2.86 | 1.43 | 0.86 | 0.15 | 0.05 |
| | | G016128 | 0.07 | 0.10 | 16.81 | 4.98 | 0.09 | 0.13 | 0.25 | 0.15 | 0.00 | 0.00 |
| | | G016133 | 1.47 | 0.46 | 1.54 | 0.56 | 60.53 | 2.58 | 1.17 | 0.34 | 0.01 | 0.02 |
| | | G016138 | 0.61 | 0.29 | 16.90 | 1.46 | 15.60 | 2.71 | 0.87 | 0.30 | 0.07 | 0.10 |
| | | G016139 | 0.01 | 0.01 | 37.25 | 5.63 | 0.44 | 0.11 | 0.20 | 0.04 | 0.00 | 0.00 |
| | | G016145 | 0.07 | 0.09 | 15.29 | 3.40 | 0.07 | 0.09 | 0.07 | 0.09 | 0.00 | 0.00 |
| | | G016146 | 0.14 | 0.20 | 12.47 | 0.70 | 1.01 | 0.25 | 0.22 | 0.10 | 0.00 | 0.00 |
| | | G016148 | 0.06 | 0.09 | 3.22 | 0.13 | 35.68 | 0.59 | 0.33 | 0.01 | 0.02 | 0.02 |
| | | G016157 | 1.43 | 0.75 | 4.90 | 0.88 | 25.16 | 6.61 | 0.71 | 0.27 | 0.58 | 0.12 |
| | | G016163 | 2.17 | 0.15 | 3.50 | 1.27 | 28.92 | 5.18 | 0.98 | 0.30 | 1.01 | 0.42 |
| | | G016164 | 0.00 | 0.00 | 3.76 | 0.36 | 52.71 | 4.20 | 0.24 | 0.34 | 0.21 | 0.16 |
| | mRNA L | G000603 | 1.79 | 0.02 | 3.53 | 0.40 | 41.24 | 8.57 | 0.89 | 0.24 | 0.23 | 0.33 |
| | | G000606 | 0.44 | 0.62 | 7.75 | 6.21 | 1.12 | 0.79 | 0.53 | 0.49 | 0.00 | 0.00 |
| | | G009577 | 1.42 | 0.68 | 5.70 | 0.55 | 25.60 | 3.13 | 1.81 | 0.54 | 0.25 | 0.09 |
| | | G016128 | 0.00 | 0.00 | 15.61 | 3.72 | 0.00 | 0.00 | 0.04 | 0.06 | 0.00 | 0.00 |
| | | G016133 | 1.39 | 1.46 | 1.50 | 2.12 | 38.86 | 21.39 | 0.43 | 0.60 | 0.07 | 0.10 |
| | | G016138 | 0.45 | 0.64 | 8.82 | 5.20 | 7.48 | 0.33 | 0.09 | 0.13 | 0.00 | 0.00 |
| | | G016139 | 0.15 | 0.21 | 19.50 | 1.06 | 0.19 | 0.15 | 0.00 | 0.00 | 0.00 | 0.00 |
| | | G016145 | 0.31 | 0.43 | 16.49 | 1.84 | 0.09 | 0.12 | 1.23 | 1.74 | 0.00 | 0.00 |
| | | G016146 | 0.00 | 0.00 | 11.28 | 4.17 | 1.68 | 0.19 | 0.36 | 0.51 | 0.00 | 0.00 |
| | | G016148 | 0.05 | 0.07 | 1.82 | 0.79 | 40.16 | 3.24 | 0.55 | 0.11 | 0.00 | 0.00 |
| | | G016157 | 1.06 | 0.10 | 4.56 | 0.31 | 32.60 | 13.25 | 0.32 | 0.23 | 0.33 | 0.24 |
| | | G016163 | 1.86 | 0.40 | 4.19 | 1.45 | 35.35 | 4.16 | 0.92 | 0.41 | 1.12 | 0.66 |
| | | G016164 | 0.00 | 0.00 | 4.67 | 0.26 | 40.10 | 20.13 | 0.20 | 0.04 | 0.27 | 0.38 |
| G021320 | mRNA A | G000267 | 0.65 | 0.32 | 2.65 | 0.23 | 53.01 | 2.06 | 0.08 | 0.12 | 0.00 | 0.00 |
| | | G000502 | 0.28 | 0.05 | 4.39 | 0.58 | 56.00 | 0.04 | 0.27 | 0.11 | 0.00 | 0.00 |
| | | G014304 | 0.36 | 0.02 | 3.84 | 0.74 | 54.36 | 8.67 | 0.13 | 0.06 | 0.00 | 0.00 |
| | | G014317 | 0.01 | 0.00 | 27.49 | 0.95 | 0.34 | 0.13 | 0.08 | 0.11 | 0.00 | 0.00 |
| | | G014322 | 0.00 | 0.01 | 29.46 | 0.21 | 0.03 | 0.04 | 0.02 | 0.03 | 0.00 | 0.00 |
| | | G014323 | 0.04 | 0.01 | 14.90 | 1.98 | 46.61 | 4.84 | 0.22 | 0.32 | 0.00 | 0.00 |
| | | G014325 | 0.01 | 0.00 | 22.10 | 0.17 | 0.00 | 0.00 | 0.24 | 0.29 | 0.00 | 0.00 |
| | | G014326 | 0.10 | 0.03 | 2.33 | 0.02 | 56.97 | 2.04 | 0.07 | 0.10 | 0.01 | 0.00 |
| | mRNA L | G000267 | 0.24 | 0.18 | 3.03 | 0.49 | 43.35 | 3.18 | 0.05 | 0.07 | 0.01 | 0.01 |
| | | G000502 | 0.21 | 0.29 | 2.57 | 2.04 | 30.16 | 23.21 | 0.09 | 0.12 | 0.00 | 0.00 |
| | | G014304 | 0.45 | 0.22 | 3.68 | 0.34 | 43.00 | 1.28 | 0.02 | 0.03 | 0.00 | 0.00 |
| | | G014317 | 0.02 | 0.00 | 11.26 | 1.36 | 0.00 | 0.00 | 0.13 | 0.19 | 0.00 | 0.00 |
| | | G014322 | 0.01 | 0.01 | 15.10 | 8.65 | 0.00 | 0.00 | 0.00 | 0.00 | 0.00 | 0.00 |
| | | G014323 | 0.02 | 0.00 | 10.18 | 2.24 | 32.37 | 0.84 | 0.35 | 0.25 | 0.00 | 0.00 |
| | | G014325 | 0.01 | 0.01 | 17.78 | 2.40 | 0.00 | 0.00 | 0.00 | 0.00 | 0.00 | 0.00 |
| | | G014326 | 0.23 | 0.23 | 2.51 | 0.01 | 52.45 | 1.95 | 0.10 | 0.14 | 0.00 | 0.00 |

TABLE 34
Mean percent editing of orthogonal Cas9-Cas9 fusion and Nme with
Nme guide only at the TTR locus in PMH (n = 2 for all groups)

| | Nme | Spy indels | | Nme2 indels | | Excisions | | Indels in both | | Inversions | |
|---|---|---|---|---|---|---|---|---|---|---|---|
| Nuclease | guide | Mean | SD | Mean | SD | Mean | SD | Mean | SD | Mean | SD |
| Orthogonal | G021320 | 0.00 | 0.00 | 36.80 | 6.58 | 0.00 | 0.00 | 0.14 | 0.20 | 0.00 | 0.00 |
| Cas9-Cas9 | G021237 | 0.01 | 0.01 | 28.97 | 7.74 | 0.02 | 0.03 | 0.15 | 0.12 | 0.00 | 0.00 |
| fusion | G021247 | 0.01 | 0.02 | 26.92 | 8.48 | 0.00 | 0.00 | 0.00 | 0.00 | 0.00 | 0.00 |
| mRNA A | G021275 | 0.01 | 0.02 | 34.30 | 7.64 | 0.04 | 0.05 | 0.25 | 0.09 | 0.00 | 0.00 |
| | G021256 | 0.01 | 0.00 | 30.89 | 0.48 | 0.01 | 0.01 | 0.07 | 0.06 | 0.00 | 0.00 |
| | G021238 | 0.02 | 0.00 | 16.19 | 2.09 | 0.00 | 0.00 | 0.00 | 0.00 | 0.00 | 0.00 |
| Orthogonal | G021320 | 0.01 | 0.00 | 45.31 | 8.64 | 0.00 | 0.00 | 0.39 | 0.31 | 0.00 | 0.00 |
| Cas9-Cas9 | G021237 | 0.01 | 0.00 | 40.24 | 7.31 | 0.00 | 0.00 | 0.09 | 0.11 | 0.00 | 0.00 |
| fusion | G021247 | 0.05 | 0.07 | 30.26 | 8.97 | 0.00 | 0.00 | 0.00 | 0.00 | 0.00 | 0.00 |
| mRNA L | G021275 | 0.02 | 0.03 | 32.13 | 5.18 | 0.07 | 0.10 | 0.11 | 0.16 | 0.00 | 0.00 |
| | G021256 | 0.00 | 0.00 | 34.68 | 2.70 | 0.05 | 0.07 | 0.26 | 0.37 | 0.00 | 0.00 |
| | G021238 | 0.03 | 0.01 | 16.46 | 2.23 | 0.00 | 0.00 | 0.00 | 0.01 | 0.00 | 0.00 |
| Nme2Cas9 | G021320 | 0.00 | 0.00 | 70.82 | 6.70 | 0.14 | 0.05 | 1.56 | 0.60 | 0.00 | 0.00 |
| mRNA F | G021237 | 0.01 | 0.00 | 68.64 | 3.14 | 0.08 | 0.01 | 0.74 | 0.95 | 0.00 | 0.00 |
| | G021247 | 0.02 | 0.03 | 70.18 | 2.19 | 0.00 | 0.00 | 0.26 | 0.17 | 0.00 | 0.00 |
| | G021275 | 0.00 | 0.00 | 71.31 | 5.80 | 0.06 | 0.01 | 1.30 | 0.16 | 0.00 | 0.00 |
| | G021256 | 0.00 | 0.00 | 74.97 | 4.93 | 0.36 | 0.10 | 1.39 | 0.02 | 0.00 | 0.00 |
| | G021238 | 0.01 | 0.00 | 63.80 | 3.43 | 0.09 | 0.13 | 0.71 | 0.10 | 0.00 | 0.00 |

### Example 12—Excision with Exemplary Spy Cas9 and Nme Cas9 Guide Designs

Additional guide scaffolds were tested for excision efficacy in primary mouse hepatocytes (PMH) at the TTR locus. Primary mouse hepatocytes, PMH (IVAL MCM114), were thawed and resuspended in 50 mL Cryopreserved Hepatocyte Recovery Media (CHRM) (Invitrogen, CM7000) followed by centrifugation. Cells were resuspended in hepatocyte medium with plating supplements: William's E Medium Plating Supplements with FBS content (Gibco, Cat. A13450). Cells were pelleted by centrifugation, resuspended in media and plated at a density of 15,000 cells/well on Bio-coat collagen I coated 96-well plates (Corning #354407). Plated cells were allowed to settle and adhere for 4-6 hours in a tissue culture incubator at 37° C. and 5% CO₂ atmosphere. After incubation cells were checked for monolayer formation, existing media was aspirated and cells were plated with 100 uL Cellartis Power Primary HEP Medium (Takara, Cat. Y20020) and left to adhere overnight prior to transfection. Cells were transfected with two LNPs prepared as described in Example 1, with a molar ratio of 50% Lipid A, 38% cholesterol, 9% DSPC, and 3% PEG2k-DMG, and delivered in Cellartis Power Primary HEP Media (Takara, Cat. Y20020) in an 8-point 3-fold dose response curve starting at 100 ng of total RNA cargo per well. One LNP comprised one guide RNA or two guide RNAs in a 1:1 weight ratio, and the second LNP comprised a nuclease mRNA. The volume ratio of gRNA:mRNA transfected to the cells was 2:1.

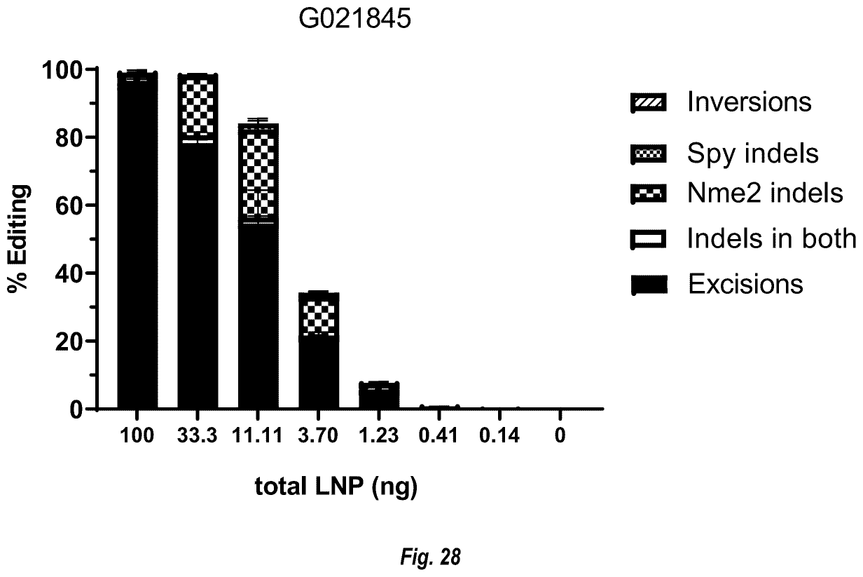

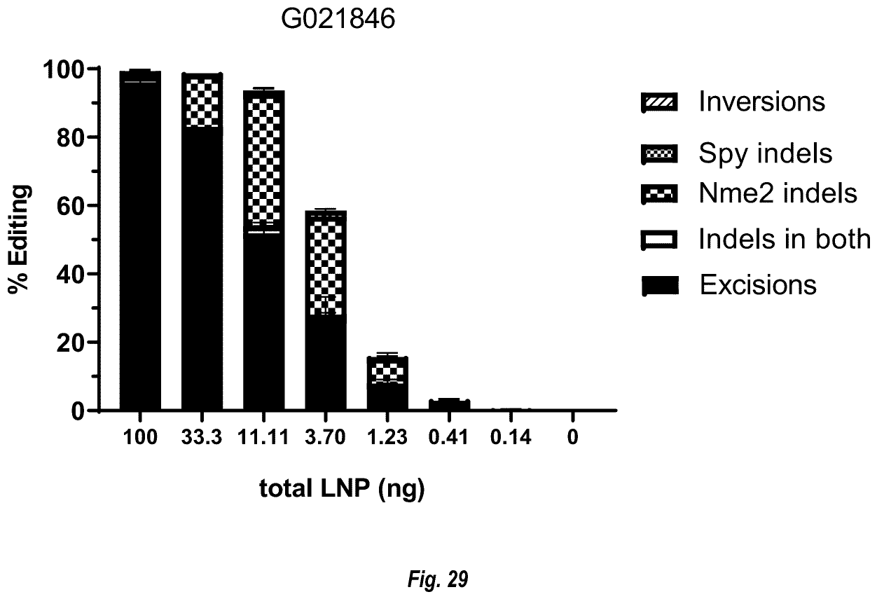

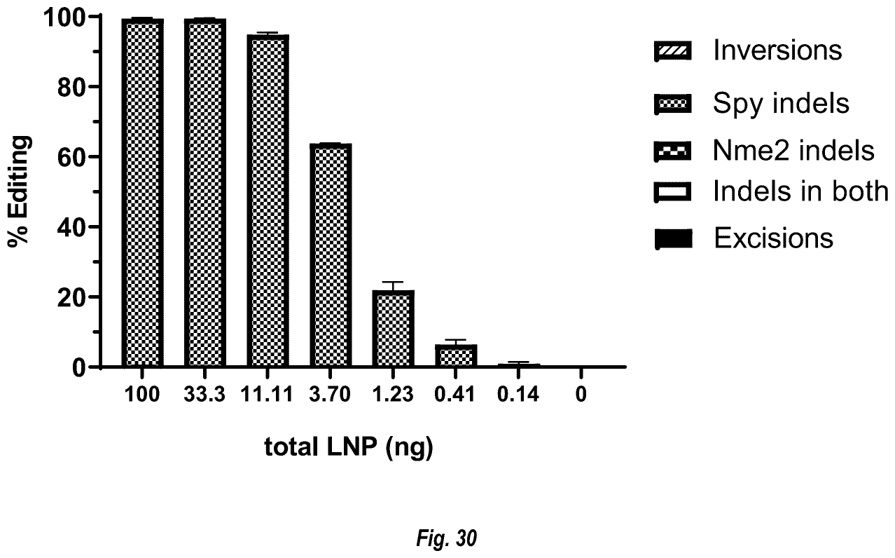

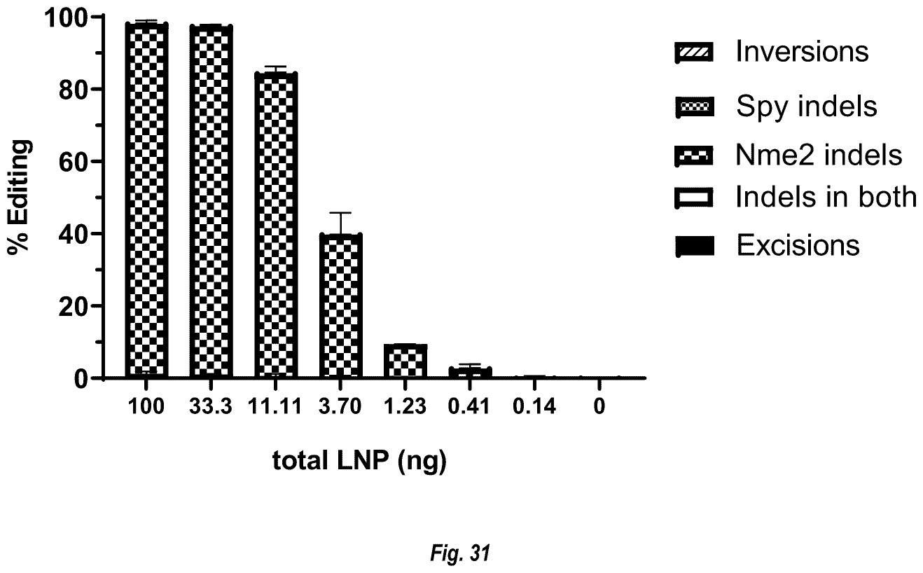

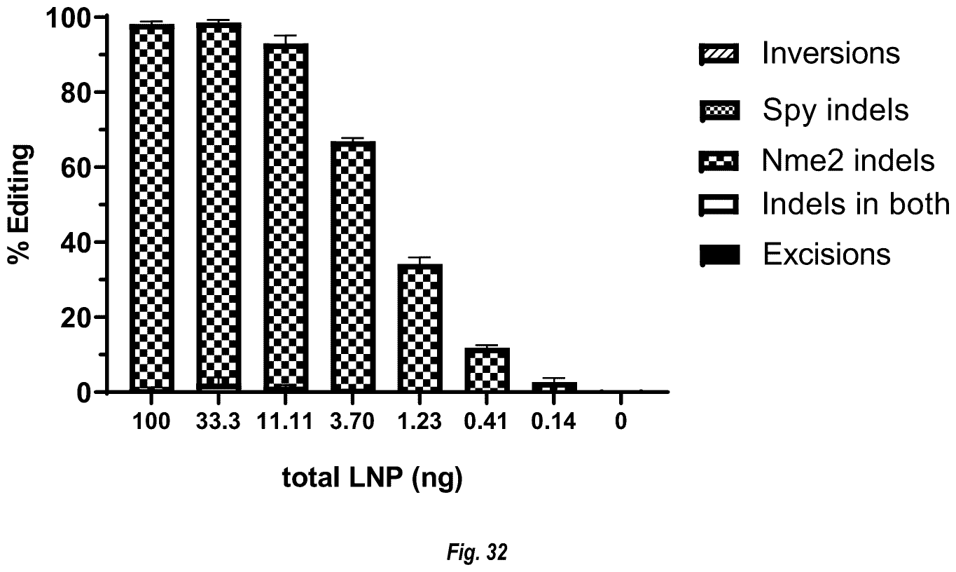

Cells were incubated for 5 days after editing. DNA isolation and NGS analysis were performed as described in Example 1. Percent editing and excision with orthogonal Cas9-Cas9 fusion constructs are shown in Table 35 and illustrated in FIG. 28 and FIG. 29. Percent editing with SpyCas9 and NmeCas9 constructs are shown in Table 36 and illustrated in FIG. 30, FIG. 31, and FIG. 32.

TABLE 35
Mean percent editing and excision with orthogonal Cas9-Cas9 fusion at the TTR locus in PMH (n = 2)

| | Spy | Nme | Total | Spy indels | | Nme2 indels | | Excisions | | Indels in both | | Inversions | |
|---|---|---|---|---|---|---|---|---|---|---|---|---|---|
| Nuclease | Guide | Guide | ng LNP | Mean | SD | Mean | SD | Mean | SD | Mean | SD | Mean | SD |
| Orthogonal | G000502 | G021845 | 100.00 | 0.10 | 0.15 | 2.36 | 0.85 | 95.23 | 0.33 | 1.36 | 0.56 | 0.00 | 0.00 |
| Cas9-Cas9 | | | 33.30 | 0.29 | 0.13 | 17.64 | 0.09 | 78.28 | 1.12 | 2.34 | 0.95 | 0.00 | 0.00 |
| fusion | | | 11.11 | 1.85 | 0.68 | 25.97 | 3.23 | 54.60 | 9.82 | 1.74 | 0.48 | 0.00 | 0.00 |
| mRNA A | | | 3.70 | 1.20 | 0.41 | 11.49 | 1.39 | 21.13 | 1.50 | 0.48 | 0.22 | 0.00 | 0.00 |
| | | | 1.23 | 0.18 | 0.05 | 2.00 | 0.53 | 5.45 | 2.29 | 0.05 | 0.08 | 0.00 | 0.00 |
| | | | 0.41 | 0.01 | 0.01 | 0.20 | 0.03 | 0.50 | 0.24 | 0.04 | 0.06 | 0.00 | 0.00 |
| | | | 0.14 | 0.01 | 0.01 | 0.03 | 0.01 | 0.02 | 0.00 | 0.04 | 0.06 | 0.00 | 0.00 |
| | | | 0.00 | 0.01 | 0.01 | 0.03 | 0.01 | 0.00 | 0.00 | 0.00 | 0.00 | 0.00 | 0.00 |
| | | G021846 | 100.00 | 0.04 | 0.06 | 1.57 | 0.51 | 96.02 | 0.85 | 1.77 | 0.08 | 0.00 | 0.00 |
| | | | 33.30 | 0.28* | N/A | 15.36* | N/A | 81.7*8 | N/A | 1.28* | N/A | 0.00* | N/A |
| | | | 11.11 | 1.04 | 0.51 | 38.77 | 1.74 | 51.89 | 2.17 | 2.04 | 1.08 | 0.00 | 0.00 |
| | | | 3.70 | 1.73 | 0.50 | 28.65 | 0.59 | 27.02 | 6.29 | 1.14 | 0.46 | 0.00 | 0.00 |
| | | | 1.23 | 0.47 | 0.27 | 7.37 | 1.58 | 7.81 | 1.28 | 0.08 | 0.07 | 0.00 | 0.00 |
| | | | 0.41 | 0.14 | 0.12 | 1.49 | 0.76 | 1.22 | 0.69 | 0.00 | 0.00 | 0.00 | 0.00 |
| | | | 0.14 | 0.03 | 0.03 | 0.28 | 0.13 | 0.06 | 0.09 | 0.00 | 0.00 | 0.00 | 0.00 |
| | | | 0.00 | 0.01 | 0.01 | 0.01 | 0.01 | 0.00 | 0.00 | 0.00 | 0.00 | 0.00 | 0.00 |

*= only one replicate

TABLE 36
Mean percent editing with Spy or NmeCas9 at the TTR locus in PMH (n = 2)

| | Spy | Nme | Total | Spy indels | | Nme2 indels | | Excisions | | Indels in both | | Inversions | |
|---|---|---|---|---|---|---|---|---|---|---|---|---|---|
| Nuclease | Guide | Guide | ng LNP | Mean | SD | Mean | SD | Mean | SD | Mean | SD | Mean | SD |
| SpyCas9 | G000502 | None | 100.00 | 99.18 | 0.31 | 0.00 | 0.00 | 0.09 | 0.13 | 0.15 | 0.22 | 0.00 | 0.00 |
| mRNA E | | | 33.30 | 99.40 | 0.10 | 0.01 | 0.02 | 0.01 | 0.02 | 0.04 | 0.01 | 0.00 | 0.00 |
| | | | 11.11 | 94.66 | 0.57 | 0.08 | 0.07 | 0.01 | 0.02 | 0.15 | 0.03 | 0.00 | 0.00 |
| | | | 3.70 | 63.64 | 0.07 | 0.00 | 0.00 | 0.00 | 0.00 | 0.16 | 0.22 | 0.00 | 0.00 |
| | | | 1.23 | 21.99 | 2.28 | 0.01 | 0.02 | 0.00 | 0.00 | 0.00 | 0.00 | 0.00 | 0.00 |
| | | | 0.41 | 6.41 | 1.32 | 0.03 | 0.02 | 0.00 | 0.00 | 0.00 | 0.00 | 0.00 | 0.00 |
| | | | 0.14 | 0.99 | 0.47 | 0.00 | 0.00 | 0.00 | 0.00 | 0.00 | 0.00 | 0.00 | 0.00 |
| | | | 0.00 | 0.02 | 0.03 | 0.03 | 0.01 | 0.00 | 0.00 | 0.00 | 0.00 | 0.00 | 0.00 |
| NmeCas9 | None | G021845 | 100.00 | 0.06 | 0.09 | 96.85 | 0.98 | 0.06 | 0.03 | 1.14 | 0.70 | 0.00 | 0.00 |
| mRNA F | | | 33.30 | 0.10 | 0.15 | 96.34 | 0.55 | 0.13 | 0.10 | 0.89 | 0.18 | 0.00 | 0.00 |
| | | | 11.11 | 0.15 | 0.18 | 83.71 | 1.99 | 0.10 | 0.07 | 0.51 | 0.57 | 0.00 | 0.00 |
| | | | 3.70 | 0.05 | 0.04 | 39.32 | 6.06 | 0.00 | 0.00 | 0.38 | 0.29 | 0.00 | 0.00 |
| | | | 1.23 | 0.00 | 0.00 | 9.43 | 0.05 | 0.00 | 0.00 | 0.01 | 0.02 | 0.00 | 0.00 |
| | | | 0.41 | 0.07 | 0.04 | 2.65 | 1.26 | 0.00 | 0.00 | 0.00 | 0.00 | 0.00 | 0.00 |
| | | | 0.14 | 0.00 | 0.00 | 0.52 | 0.11 | 0.00 | 0.00 | 0.00 | 0.00 | 0.00 | 0.00 |
| | | | 0.00 | 0.05 | 0.04 | 0.02 | 0.03 | 0.07 | 0.10 | 0.00 | 0.00 | 0.00 | 0.00 |
| | | G021846 | 100.00 | 0.00 | 0.00 | 97.43 | 0.61 | 0.05 | 0.04 | 0.77 | 0.44 | 0.00 | 0.00 |
| | | | 33.30 | 0.00 | 0.00 | 96.29 | 0.65 | 0.01 | 0.02 | 2.32 | 1.61 | 0.00 | 0.00 |
| | | | 11.11 | 0.00 | 0.00 | 91.62 | 2.08 | 0.10 | 0.12 | 1.33 | 0.46 | 0.00 | 0.00 |
| | | | 3.70 | 0.01 | 0.01 | 66.30 | 0.81 | 0.01 | 0.01 | 0.66 | 0.05 | 0.00 | 0.00 |
| | | | 1.23 | 0.00 | 0.00 | 34.01 | 1.72 | 0.03 | 0.05 | 0.16 | 0.03 | 0.00 | 0.00 |
| | | | 0.41 | 0.01 | 0.01 | 11.73 | 0.68 | 0.00 | 0.00 | 0.11 | 0.16 | 0.00 | 0.00 |
| | | | 0.14 | 0.02 | 0.00 | 2.67 | 1.06 | 0.00 | 0.00 | 0.05 | 0.06 | 0.00 | 0.00 |
| | | | 0.00 | 0.00 | 0.00 | 0.09 | 0.08 | 0.00 | 0.00 | 0.00 | 0.00 | 0.00 | 0.00 |

In the following tables of sequences, the terms “mA,” “mC,” “mU,” or “mG” are used to denote a nucleotide that has been modified with 2′-O-Me. In the following table, each “N” is used to independently denote any nucleotide (e.g., A, U, T, C, G). In certain embodiments, the nucleotide is an unmodified RNA nucleotide residue, i.e., a ribose sugar and a phosphodiester backbone. In the following table, a “*” is used to denote a PS modification. In this application, the terms A*, C*, U*, or G* may be used to denote a nucleotide that is linked to the next (e.g., 3′) nucleotide with a PS bond. It is understood that if a DNA sequence (comprising Ts) is referenced with respect to an RNA, then Ts should be replaced with Us (which may be modified or unmodified depending on the context), and vice versa. In the following table, single amino acid letter code is used to provide peptide sequences.

TABLE 37
Table of Sequences

| SEQ ID | | |
|---|---|---|
| NO | Description | sequence |
| 1 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | (mRNA A)- | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | mRNA | GGAGGAGGACAAGAAGCACGAGCGGCACCCCAUCUUCGGCAACAUCGUGGACGAGGUGGCCUACCACGAGAAGUACCCCACCAUCUACCAC |
| | sequence | CUGCGGAAGAAGCUGGUGGACUCCACCGACAAGGCCGACCUGCGGCUGAUCUACCUGGCCCUGGCCCACAUGAUCAAGUUCCGGGGCCACU |
| | | UCCUGAUCGAGGGCGACCUGAACCCCGACAACUCCGACGUGGACAAGCUGUUCAUCCAGCUGGUGCAGACCUACAACCAGCUGUUCGAGGA |
| | | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | | CCGAGGACGCCAAGCUGCAGCUGUCCAAGGACACCUACGACGACGACCUGGACAACCUGCUGGCCCAGAUCGGCGACCAGUACGCCGACCU |
| | | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | | UCCAUGAUCAAGCGGUACGACGAGCACCACCAGGACCUGACCCUGCUGAAGGCCCUGGUGCGGCAGCAGCUGCCCGAGAAGUACAAGGAGA |
| | | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGGAGGACCUGCUGCGGAAGCAGCGGACCUUCGACAACGGCUCCAUC |
| | | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGCGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCACCGAGGAGGGCACCUCC |
| | | ACCGAGUCCGAGGGCUCCGCCGGCACCUCCACCGAGUCCGAGGGCUCCGCCGGCACCUCCGAGUCCGCCACCGAGUCCGGCGGCACCUCCA |
| | | CCGAGUCCGAGGGCUCCUCCUCCGGUGCCGCCUUCAAGCCCAACCCCAUCAACUACAUCCUGGGCCUGGACAUCGGCAUCGCCUCCGUGGG |
| | | CUGGGCCAUGGUGGAGAUCGACGAGGAGGAGAACCCCAUCCGGCUGAUCGACCUGGGCGUGCGGGUGUUCGAGCGGGCCGAGGUGCCCAAG |
| | | ACCGGCGACUCCCUGGCCAUGGCCCGGCGGCUGGCCCGGUCCGUGCGGCGGCUGACCCGGGGGGGGGCCCACCGGCUGCUGCGGGCCCGGC |
| | | GGCUGCUGAAGCGGGAGGGCGUGCUGCAGGCCGCCGACUUCGACGAGAACGGCCUGAUCAAGUCCCUGCCCAACACCCCCUGGCAGCUGCG |
| | | GGCCGCCGCCCUGGACCGGAAGCUGACCCCCCUGGAGUGGUCCGCCGUGCUGCUGCACCUGAUCAAGCACCGGGGCUACCUGUCCCAGCGG |
| | | AAGAACGAGGGCGAGACCGCCGACAAGGAGCUGGGCGCCCUGCUGAAGGGCGUGGCCAACAACGCCCACGCCCUGCAGACCGGCGACUUCC |
| | | GGACCCCCGCCGAGCUGGCCCUGAACAAGUUCGAGAAGGAGUCCGGCCACAUCCGGAACCAGCGGGGCGACUACUCCCACACCUUCUCCCG |
| | | GAAGGACCUGCAGGCCGAGCUGAUCCUGCUGUUCGAGAAGCAGAAGGAGUUCGGCAACCCCCACGUGUCCGGCGGCCUGAAGGAGGGCAUC |
| | | GAGACCCUGCUGAUGACCCAGCGGCCCGCCCUGUCCGGCGACGCCGUGCAGAAGAUGCUGGGCCACUGCACCUUCGAGCCCGCCGAGCCCA |
| | | AGGCCGCCAAGAACACCUACACCGCCGAGCGGUUCAUCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAGCUGACCUACGCCCAGGCCCGGAAGCUGCUGGGCCUG |
| | | GAGGACACCGCCUUCUUCAAGGGCCUGCGGUACGGCAAGGACAACGCCGAGGCCUCCACCCUGAUGGAGAUGAAGGCCUACCACGCCAUCU |
| | | CCCGGGCCCUGGAGAAGGAGGGCCUGAAGGACAAGAAGUCCCCCCUGAACCUGUCCUCCGAGCUGCAGGACGAGAUCGGCACCGCCUUCUC |
| | | CCUGUUCAAGACCGACGAGGACAUCACCGGCCGGCUGAAGGACCGGGUGCAGCCCGAGAUCCUGGAGGCCCUGCUGAAGCACAUCUCCUUC |
| | | GACAAGUUCGUGCAGAUCUCCCUGAAGGCCCUGCGGCGGAUCGUGCCCCUGAUGGAGCAGGGCAAGCGGUACGACGAGGCCUGCGCCGAGA |
| | | UCUACGGCGACCACUACGGCAAGAAGAACACCGAGGAGAAGAUCUACCUGCCCCCCAUCCCCGCCGACGAGAUCCGGAACCCCGUGGUGCU |
| | | GCGGGCCCUGUCCCAGGCCCGGAAGGUGAUCAACGGCGUGGUGCGGCGGUACGGCUCCCCCGCCCGGAUCCACAUCGAGACCGCCCGGGAG |
| | | GUGGGCAAGUCCUUCAAGGACCGGAAGGAGAUCGAGAAGCGGCAGGAGGAGAACCGGAAGGACCGGGAGAAGGCCGCCGCCAAGUUCCGGG |
| | | AGUACUUCCCCAACUUCGUGGGCGAGCCCAAGUCCAAGGACAUCCUGAAGCUGCGGCUGUACGAGCAGCAGCACGGCAAGUGCCUGUACUC |
| | | CGGCAAGGAGAUCAACCUGGUGCGGCUGAACGAGAAGGGCUACGUGGAGAUCGACCACGCCCUGCCCUUCUCCCGGACCUGGGACGACUCC |
| | | UUCAACAACAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUCGACGAGGA |
| | | CGGCUUCAAGGAGUGCAACCUGAACGACACCCGGUACGUGAACCGCUUCCUGUGCCAGUUCGUGGCCGACCACAUCCUGCUGACCGGCAAG |
| | | GGCAAGCGGCGGGUGUUCGCCUCCAACGGCCAGAUCACCAACCUGCUGCGGGGCUUCUGGGGCCUGCGGAAGGUGCGGGCCGAGAACGACC |
| | | GGCACCACGCCCUGGACGCCGUGGUGGUGGCCUGCUCCACCGUGGCCAUGCAGCAGAAGAUCACCCGGUUCGUGCGGUACAAGGAGAUGAA |
| | | CGCCUUCGACGGCAAGACCAUCGACAAGGAGACCGGCAAGGUGCUGCACCAGAAGACCCACUUCCCCCAGCCCUGGGAGUUCUUCGCCCAG |
| | | GAGGUGAUGAUCCGGGUGUUCGGCAAGCCCGACGGCAAGCCCGAGUUCGAGGAGGCCGACACCCCCGAGAAGCUGCGGACCCUGCUGGCCG |
| | | AGAAGCUGUCCUCCCGGCCCGAGGCCGUGCACGAGUACGUGACCCCCCUGUUCGUGUCCCGGGCCCCCAACCGGAAGAUGUCCGGCGCCCA |
| | | CAAGGACACCCUGCGGUCCGCCAAGCGGUUCGUGAAGCACAACGAGAAGAUCUCCGUGAAGCGGGUGUGGCUGACCGAGAUCAAGCUGGCC |
| | | GACCUGGAGAACAUGGUGAACUACAAGAACGGCCGGGAGAUCGAGCUGUACGAGGCCCUGAAGGCCCGGCUGGAGGCCUACGGCGGCAACG |
| | | CCAAGCAGGCCUUCGACCCCAAGGACAACCCCUUCUACAAGAAGGGCGGCCAGCUGGUGAAGGCCGUGCGGGUGGAGAAGACCCAGGAGUC |
| | | CGGCGUGCUGCUGAACAAGAAGAACGCCUACACCAUCGCCGACAACGGCGACAUGGUGCGGGUGGACGUGUUCUGCAAGGUGGACAAGAAG |
| | | GGCAAGAACCAGUACUUCAUCGUGCCCAUCUACGCCUGGCAGGUGGCCGAGAACAUCCUGCCCGACAUCGACUGCAAGGGCUACCGGAUCG |
| | | ACGACUCCUACACCUUCUGCUUCUCCCUGCACAAGUACGACCUGAUCGCCUUCCAGAAGGACGAGAAGUCCAAGGUGGAGUUCGCCUACUA |
| | | CAUCAACUGCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCGGUCCGGAA |
| | | AGCGGACCGCCGACGGCUCCGAGUUCGAGUCCCCCAAGAAGAAGCGGAAGGUGGAGUAGCUAGCACCAGCCUCAAGAACACCCGAAUGGAG |
| | | UCUCUAAGCUACAUAAUACCAACUUACACUUUACAAAAUGUUGUCCCCCAAAAUGUAGCCAUUCGUAUCUGCUCCUAAUAAAAAGAAAGUU |
| | | UCUUCACAUUCUCUCGAGAAAAAAAAAAAAUGGAAAAAAAAAAAACGGAAAAAAAAAAAAGGUAAAAAAAAAAAAUAUAAAAAAAAAAAAC |
| | | AUAAAAAAAAAAAACGAAAAAAAAAAAACGUAAAAAAAAAAAACUCAAAAAAAAAAAAGAUAAAAAAAAAAAACCUAAAAAAAAAAAAUGU |
| | | AAAAAAAAAAAAGGGAAAAAAAAAAAACGCAAAAAAAAAAAACACAAAAAAAAAAAAUGCAAAAAAAAAAAAUCGAAAAAAAAAAAAUCUA |
| | | AAAAAAAAAAACGAAAAAAAAAAAACCCAAAAAAAAAAAAGACAAAAAAAAAAAAUAGAAAAAAAAAAAGUUAAAAAAAAAAAACUGAAAA |
| | | AAAAAAAAUUUAAAAAAAAAAAAUCUAG |
| 2 | Cas9- | auggacaagaaguacuccaucggccuggacaucggcaccaacuccgugggcugggccgugaucaccgacgaguacaaggugcccuccaaga |
| | Cas9 | aguucaaggugcugggcaacaccgaccggcacuccaucaagaagaaccugaucggcgcccugcuguucgacuccggcgagaccgccgaggc |
| | fusion | cacccggcugaagcggaccgcccggcggcgguacacccggcggaagaaccggaucugcuaccugcaggagaucuucuccaacgagauggcc |
| | (mRNA A)- | aagguggacgacuccuucuuccaccggcuggaggaguccuuccugguggaggaggacaagaagcacgagcggcaccccaucuucggcaaca |
| | ORF | ucguggacgagguggccuaccacgagaaguaccccaccaucuaccaccugcggaagaagcugguggacuccaccgacaaggccgaccugcg |
| | sequence | gcugaucuaccuggcccuggcccacaugaucaaguuccggggccacuuccugaucgagggcgaccugaaccccgacaacuccgacguggac |
| | | aagcuguucauccagcuggugcagaccuacaaccagcuguucgaggagaaccccaucaacgccuccggcguggacgccaaggccauccugu |
| | | ccgcccggcuguccaagucccggcggcuggagaaccugaucgcccagcugcccggcgagaagaagaacggccuguucggcaaccugaucgc |
| | | ccugucccugggccugacccccaacuucaaguccaacuucgaccuggccgaggacgccaagcugcagcuguccaaggacaccuacgacgac |
| | | gaccuggacaaccugcuggcccagaucggcgaccaguacgccgaccuguuccuggccgccaagaaccuguccgacgccauccugcuguccg |
| | | acauccugcgggugaacaccgagaucaccaaggccccccuguccgccuccaugaucaagcgguacgacgagcaccaccaggaccugacccu |
| | | gcugaaggcccuggugcggcagcagcugcccgagaaguacaaggagaucuucuucgaccaguccaagaacggcuacgccggcuacaucgac |
| | | ggcggcgccucccaggaggaguucuacaaguucaucaagcccauccuggagaagauggacggcaccgaggagcugcuggugaagcugaacc |
| | | gggaggaccugcugcggaagcagcggaccuucgacaacggcuccaucccccaccagauccaccugggcgagcugcacgccauccugcggcg |
| | | gcaggaggacuucuaccccuuccugaaggacaaccgggagaagaucgagaagauccugaccuuccggauccccuacuacgugggcccccug |
| | | gcccggggcaacucccgguucgccuggaugacccggaaguccgaggagaccaucacccccuggaacuucgaggaggugguggacaagggcg |
| | | ccuccgcccaguccuucaucgagcggaugaccaacuucgacaagaaccugcccaacgagaaggugcugcccaagcacucccugcuguacga |
| | | guacuucaccguguacaacgagcugaccaaggugaaguacgugaccgagggcaugcggaagcccgccuuccuguccggcgagcagaagaag |
| | | gccaucguggaccugcuguucaagaccaaccggaaggugaccgugaagcagcugaaggaggacuacuucaagaagaucgagugcuucgacu |
| | | ccguggagaucuccggcguggaggaccgguucaacgccucccugggcaccuaccacgaccugcugaagaucaucaaggacaaggacuuccu |
| | | ggacaacgaggagaacgaggacauccuggaggacaucgugcugacccugacccuguucgaggaccgggagaugaucgaggagcggcugaag |
| | | accuacgcccaccuguucgacgacaaggugaugaagcagcugaagcggcggcgguacaccggcuggggccggcugucccggaagcugauca |
| | | acggcauccgggacaagcaguccggcaagaccauccuggacuuccugaaguccgacggcuucgccaaccggaacuucaugcagcugaucca |
| | | cgacgacucccugaccuucaaggaggacauccagaaggcccagguguccggccagggcgacucccugcacgagcacaucgccaaccuggcc |
| | | ggcucccccgccaucaagaagggcauccugcagaccgugaaggugguggacgagcuggugaaggugaugggccggcacaagcccgagaaca |
| | | ucgugaucgagauggcccgggagaaccagaccacccagaagggccagaagaacucccgggagcggaugaagcggaucgaggagggcaucaa |
| | | ggagcugggcucccagauccugaaggagcaccccguggagaacacccagcugcagaacgagaagcuguaccuguacuaccugcagaacggc |
| | | cgggacauguacguggaccaggagcuggacaucaaccggcuguccgacuacgacguggaccacaucgugccccaguccuuccugaaggacg |
| | | acuccaucgacaacaaggugcugacccgguccgacaagaaccggggcaaguccgacaacgugcccuccgaggagguggugaagaagaugaa |
| | | gaacuacuggcggcagcugcugaacgccaagcugaucacccagcggaaguucgacaaccugaccaaggccgagcggggcggccuguccgag |
| | | cuggacaaggccggcuucaucaagcggcagcugguggagacccggcagaucaccaagcacguggcccagauccuggacucccggaugaaca |
| | | ccaaguacgacgagaacgacaagcugauccgggaggugaaggugaucacccugaaguccaagcugguguccgacuuccggaaggacuucca |
| | | guucuacaaggugcgggagaucaacaacuaccaccacgcccacgacgccuaccugaacgccguggugggcaccgcccugaucaagaaguac |
| | | cccaagcuggaguccgaguucguguacggcgacuacaagguguacgacgugcggaagaugaucgccaaguccgagcaggagaucggcaagg |
| | | ccaccgccaaguacuucuucuacuccaacaucaugaacuucuucaagaccgagaucacccuggccaacggcgagauccggaagcggccccu |
| | | gaucgagaccaacggcgagaccggcgagaucgugugggacaagggccgggacuucgccaccgugcggaaggugcuguccaugccccaggug |
| | | aacaucgugaagaagaccgaggugcagaccggcggcuucuccaaggaguccauccugcccaagcggaacuccgacaagcugaucgcccgga |
| | | agaaggacugggaccccaagaaguacggcggcuucgacucccccaccguggccuacuccgugcuggugguggccaagguggagaagggcaa |
| | | guccaagaagcugaaguccgugaaggagcugcugggcaucaccaucauggagcgguccuccuucgagaagaaccccaucgacuuccuggag |
| | | gccaagggcuacaaggaggugaagaaggaccugaucaucaagcugcccaaguacucccuguucgagcuggagaacggccggaagcggaugc |
| | | uggccuccgccggcgagcugcagaagggcaacgagcuggcccugcccuccaaguacgugaacuuccuguaccuggccucccacuacgagaa |
| | | gcugaagggcucccccgaggacaacgagcagaagcagcuguucguggagcagcacaagcacuaccuggacgagaucaucgagcagaucucc |
| | | gaguucuccaagcgggugauccuggccgacgccaaccuggacaaggugcuguccgccuacaacaagcaccgggacaagcccauccgggagc |
| | | aggccgagaacaucauccaccuguucacccugaccaaccugggcgcccccgccgccuucaaguacuucgacaccaccaucgacAAGaagcg |
| | | guacaccuccaccaaggaggugcuggacgccacccugauccaccaguccaucaccggccuguacgagacccggaucgaccugucccagcug |
| | | ggcggcgacGGCgccCCCgagUCCgccACCgagUCCggcGGCaccUCCaccGAGuccGAGggcUCCgccGGCaccUCCaccGAGuccGAGg |
| | | gcuccGCCggcUCCgccGGCuccaccUCCaccGAGgagGGCaccUCCaccGAGuccGAGggcuccGCCggcACCuccACCgaguccgagGG |
| | | CuccGCCggcACCuccGAGuccgccACCgagUCCggcGGCaccUCCaccGAGuccGAGggcUCCuccUCCggugccgccuucaagcccaac |
| | | cccaucaacuacauccugggccuggacaucggcaucgccuccgugggcugggccaugguggagaucgacgaggaggagaaccccauccggc |
| | | ugaucgaccugggcgugcggguguucgagcgggccgaggugcccaagaccggcgacucccuggccauggcccggcggcuggcccgguccgu |
| | | gcggcggcugacccggcggcgggcccaccggcugcugcgggcccggcggcugcugaagcgggagggcgugcugcaggccgccgacuucgac |
| | | gagaacggccugaucaagucccugcccaacacccccuggcagcugcgggccgccgcccuggaccggaagcugaccccccuggagugguccg |
| | | ccgugcugcugcaccugaucaagcaccggggcuaccugucccagcggaagaacgagggcgagaccgccgacaaggagcugggcgcccugcu |
| | | gaagggcguggccaacaacgcccacgcccugcagaccggcgacuuccggacccccgccgagcuggcccugaacaaguucgagaaggagucc |
| | | ggccacauccggaaccagcggggcgacuacucccacaccuucucccggaaggaccugcaggccgagcugauccugcuguucgagaagcaga |
| | | aggaguucggcaacccccacguguccggcggccugaaggagggcaucgagacccugcugaugacccagcggcccgcccuguccggcgacgc |
| | | cgugcagaagaugcugggccacugcaccuucgagcccgccgagcccaaggccgccaagaacaccuacaccgccgagcgguucaucuggcug |
| | | accaagcugaacaaccugcggauccuggagcagggcuccgagcggccccugaccgacaccgagcgggccacccugauggacgagcccuacc |
| | | ggaaguccaagcugaccuacgcccaggcccggaagcugcugggccuggaggacaccgccuucuucaagggccugcgguacggcaaggacaa |
| | | cgccgaggccuccacccugauggagaugaaggccuaccacgccaucucccgggcccuggagaaggagggccugaaggacaagaaguccccc |
| | | cugaaccuguccuccgagcugcaggacgagaucggcaccgccuucucccuguucaagaccgacgaggacaucaccggccggcugaaggacc |
| | | gggugcagcccgagauccuggaggcccugcugaagcacaucuccuucgacaaguucgugcagaucucccugaaggcccugcggcggaucgu |
| | | gccccugauggagcagggcaagcgguacgacgaggccugcgccgagaucuacggcgaccacuacggcaagaagaacaccgaggagaagauc |
| | | uaccugccccccauccccgccgacgagauccggaaccccguggugcugcgggcccugucccaggcccggaaggugaucaacggcguggugc |
| | | ggcgguacggcucccccgcccggauccacaucgagaccgcccgggaggugggcaaguccuucaaggaccggaaggagaucgagaagcggca |
| | | ggaggagaaccggaaggaccgggagaaggccgccgccaaguuccgggaguacuuccccaacuucgugggcgagcccaaguccaaggacauc |
| | | cugaagcugcggcuguacgagcagcagcacggcaagugccuguacuccggcaaggagaucaaccuggugcggcugaacgagaagggcuacg |
| | | uggagaucgaccacgcccugcccuucucccggaccugggacgacuccuucaacaacaaggugcuggugcugggcuccgagaaccagaacaa |
| | | gggcaaccagacccccuacgaguacuucaacggcaaggacaacucccgggaguggcaggaguucaaggcccggguggagaccucccgguuc |
| | | ccccgguccaagaagcagcggauccugcugcagaaguucgacgaggacggcuucaaggagugcaaccugaacgacacccgguacgugaacc |
| | | gcuuccugugccaguucguggccgaccacauccugcugaccggcaagggcaagcggcggguguucgccuccaacggccagaucaccaaccu |
| | | gcugcggggcuucuggggccugcggaaggugcgggccgagaacgaccggcaccacgcccuggacgccguggugguggccugcuccaccgug |
| | | gccaugcagcagaagaucacccgguucgugcgguacaaggagaugaacgccuucgacggcaagaccaucgacaaggagaccggcaaggugc |
| | | ugcaccagaagacccacuucccccagcccugggaguucuucgcccaggaggugaugauccggguguucggcaagcccgacggcaagcccga |
| | | guucgaggaggccgacacccccgagaagcugcggacccugcuggccgagaagcuguccucccggcccgaggccgugcacgaguacgugacc |
| | | ccccuguucgugucccgggcccccaaccggaagauguccggcgcccacaaggacacccugcgguccgccaagcgguucgugaagcacaacg |
| | | agaagaucuccgugaagcggguguggcugaccgagaucaagcuggccgaccuggagaacauggugaacuacaagaacggccgggagaucga |
| | | gcuguacgaggcccugaaggcccggcuggaggccuacggcggcaacgccaagcaggccuucgaccccaaggacaaccccuucuacaagaag |
| | | ggcggccagcuggugaaggccgugcggguggagaagacccaggaguccggcgugcugcugaacaagaagaacgccuacaccaucgccgaca |
| | | acggcgacauggugcggguggacguguucugcaagguggacaagaagggcaagaaccaguacuucaucgugcccaucuacgccuggcaggu |
| | | ggccgagaacauccugcccgacaucgacugcaagggcuaccggaucgacgacuccuacaccuucugcuucucccugcacaaguacgaccug |
| | | aucgccuuccagaaggacgagaaguccaagguggaguucgccuacuacaucaacugcgacuccuccaacggccgguucuaccuggccuggc |
| | | acgacaagggcuccaaggagcagcaguuccggaucuccacccagaaccuggugcugauccagaaguaccaggugaacgagcugggcaagga |
| | | gauccggcccugccggcugaagaagcggccccccgugcgguccggaaagcggaccgccgacggcuccgaguucgagucccccaagaagaag |
| | | cggaagguggaguag |
| 3 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion | KLFIQLVQTYNQLFEENPINASGVDAKAILSARLSKSRRLENLIAQLPGEKKNGLFGNLIALSLGLTPNFKSNFDLAEDAKLQLSKDTYDD |
| | (mRNA A)- | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | sequence | AIVDLLFKTNRKVTVKQLKEDYFKKIECFDSVEISGVEDRFNASLGTYHDLLKIIKDKDFLDNEENEDILEDIVLTLTLFEDREMIEERLK |
| | | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | PINYILGLDIGIASVGWAMVEIDEEENPIRLIDLGVRVFERAEVPKTGDSLAMARRLARSVRRLTRRRAHRLLRARRLLKREGVLQAADFD |
| | | ENGLIKSLPNTPWQLRAAALDRKLTPLEWSAVLLHLIKHRGYLSQRKNEGETADKELGALLKGVANNAHALQTGDFRTPAELALNKFEKES |
| | | GHIRNQRGDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLVRLNEKGYVEIDHALPFSRTWDDSFNNKVLVLGSENQNKGNQTPYEYFNGKDNSREWQEFKARVETSRF |
| | | PRSKKQRILLQKFDEDGFKECNLNDTRYVNRFLCQFVADHILLTGKGKRRVFASNGQITNLLRGFWGLRKVRAENDRHHALDAVVVACSTV |
| | | AMQQKITRFVRYKEMNAFDGKTIDKETGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEALKARLEAYGGNAKQAFDPKDNPFYKK |
| | | GGQLVKAVRVEKTQESGVLLNKKNAYTIADNGDMVRVDVFCKVDKKGKNQYFIVPIYAWQVAENILPDIDCKGYRIDDSYTFCFSLHKYDL |
| | | IAFQKDEKSKVEFAYYINCDSSNGRFYLAWHDKGSKEQQFRISTQNLVLIQKYQVNELGKEIRPCRLKKRPPVRSGKRTADGSEFESPKKK |
| | | RKVE* |
| 4 | Cas9- | auggacaagaaguacuccaucggccuggacaucggcaccaacuccgugggcugggccgugaucaccgacgaguacaaggugcccuccaaga |
| | Cas9 | aguucaaggugcugggcaacaccgaccggcacuccaucaagaagaaccugaucggcgcccugcuguucgacuccggcgagaccgccgaggc |
| | fusion | cacccggcugaagcggaccgcccggcggcgguacacccggcggaagaaccggaucugcuaccugcaggagaucuucuccaacgagauggcc |
| | (mRNA A | aagguggacgacuccuucuuccaccggcuggaggaguccuuccugguggaggaggacaagaagcacgagcggcaccccaucuucggcaaca |
| | with | ucguggacgagguggccuaccacgagaaguaccccaccaucuaccaccugcggaagaagcugguggacuccaccgacaaggccgaccugcg |
| | HiBit | gcugaucuaccuggcccuggcccacaugaucaaguuccggggccacuuccugaucgagggcgaccugaaccccgacaacuccgacguggac |
| | tag)- | aagcuguucauccagcuggugcagaccuacaaccagcuguucgaggagaaccccaucaacgccuccggcguggacgccaaggccauccugu |
| | ORF | ccgcccggcuguccaagucccggcggcuggagaaccugaucgcccagcugcccggcgagaagaagaacggccuguucggcaaccugaucgc |
| | sequence | ccugucccugggccugacccccaacuucaaguccaacuucgaccuggccgaggacgccaagcugcagcuguccaaggacaccuacgacgac |
| | | gaccuggacaaccugcuggcccagaucggcgaccaguacgccgaccuguuccuggccgccaagaaccuguccgacgccauccugcuguccg |
| | | acauccugcgggugaacaccgagaucaccaaggccccccuguccgccuccaugaucaagcgguacgacgagcaccaccaggaccugacccu |
| | | gcugaaggcccuggugcggcagcagcugcccgagaaguacaaggagaucuucuucgaccaguccaagaacggcuacgccggcuacaucgac |
| | | ggcggcgccucccaggaggaguucuacaaguucaucaagcccauccuggagaagauggacggcaccgaggagcugcuggugaagcugaacc |
| | | gggaggaccugcugcggaagcagcggaccuucgacaacggcuccaucccccaccagauccaccugggcgagcugcacgccauccugcggcg |
| | | gcaggaggacuucuaccccuuccugaaggacaaccgggagaagaucgagaagauccugaccuuccggauccccuacuacgugggcccccug |
| | | gcccggggcaacucccgguucgccuggaugacccggaaguccgaggagaccaucacccccuggaacuucgaggaggugguggacaagggcg |
| | | ccuccgcccaguccuucaucgagcggaugaccaacuucgacaagaaccugcccaacgagaaggugcugcccaagcacucccugcuguacga |
| | | guacuucaccguguacaacgagcugaccaaggugaaguacgugaccgagggcaugcggaagcccgccuuccuguccggcgagcagaagaag |
| | | gccaucguggaccugcuguucaagaccaaccggaaggugaccgugaagcagcugaaggaggacuacuucaagaagaucgagugcuucgacu |
| | | ccguggagaucuccggcguggaggaccgguucaacgccucccugggcaccuaccacgaccugcugaagaucaucaaggacaaggacuuccu |
| | | ggacaacgaggagaacgaggacauccuggaggacaucgugcugacccugacccuguucgaggaccgggagaugaucgaggagcggcugaag |
| | | accuacgcccaccuguucgacgacaaggugaugaagcagcugaagcggcggcgguacaccggcuggggccggcugucccggaagcugauca |
| | | acggcauccgggacaagcaguccggcaagaccauccuggacuuccugaaguccgacggcuucgccaaccggaacuucaugcagcugaucca |
| | | cgacgacucccugaccuucaaggaggacauccagaaggcccagguguccggccagggcgacucccugcacgagcacaucgccaaccuggcc |
| | | ggcucccccgccaucaagaagggcauccugcagaccgugaaggugguggacgagcuggugaaggugaugggccggcacaagcccgagaaca |
| | | ucgugaucgagauggcccgggagaaccagaccacccagaagggccagaagaacucccgggagcggaugaagcggaucgaggagggcaucaa |
| | | ggagcugggcucccagauccugaaggagcaccccguggagaacacccagcugcagaacgagaagcuguaccuguacuaccugcagaacggc |
| | | cgggacauguacguggaccaggagcuggacaucaaccggcuguccgacuacgacguggaccacaucgugccccaguccuuccugaaggacg |
| | | acuccaucgacaacaaggugcugacccgguccgacaagaaccggggcaaguccgacaacgugcccuccgaggagguggugaagaagaugaa |
| | | gaacuacuggcggcagcugcugaacgccaagcugaucacccagcggaaguucgacaaccugaccaaggccgagcggcggggccuguccgag |
| | | cuggacaaggccggcuucaucaagcggcagcugguggagacccggcagaucaccaagcacguggcccagauccuggacucccggaugaaca |
| | | ccaaguacgacgagaacgacaagcugauccgggaggugaaggugaucacccugaaguccaagcugguguccgacuuccggaaggacuucca |
| | | guucuacaaggugcgggagaucaacaacuaccaccacgcccacgacgccuaccugaacgccguggugggcaccgcccugaucaagaaguac |
| | | cccaagcuggaguccgaguucguguacggcgacuacaagguguacgacgugcggaagaugaucgccaaguccgagcaggagaucggcaagg |
| | | ccaccgccaaguacuucuucuacuccaacaucaugaacuucuucaagaccgagaucacccuggccaacggcgagauccggaagcggccccu |
| | | gaucgagaccaacggcgagaccggcgagaucgugugggacaagggccgggacuucgccaccgugcggaaggugcuguccaugccccaggug |
| | | aacaucgugaagaagaccgaggugcagaccggcggcuucuccaaggaguccauccugcccaagcggaacuccgacaagcugaucgcccgga |
| | | agaaggacugggaccccaagaaguacggcggcuucgacucccccaccguggccuacuccgugcuggugguggccaagguggagaagggcaa |
| | | guccaagaagcugaaguccgugaaggagcugcugggcaucaccaucauggagcgguccuccuucgagaagaaccccaucgacuuccuggag |
| | | gccaagggcuacaaggaggugaagaaggaccugaucaucaagcugcccaaguacucccuguucgagcuggagaacggccggaagcggaugc |
| | | uggccuccgccggcgagcugcagaagggcaacgagcuggcccugcccuccaaguacgugaacuuccuguaccuggccucccacuacgagaa |
| | | gcugaagggcucccccgaggacaacgagcagaagcagcuguucguggagcagcacaagcacuaccuggacgagaucaucgagcagaucucc |
| | | gaguucuccaagcgggugauccuggccgacgccaaccuggacaaggugcuguccgccuacaacaagcaccgggacaagcccauccgggagc |
| | | aggccgagaacaucauccaccuguucacccugaccaaccugggcgcccccgccgccuucaaguacuucgacaccaccaucgacAAGaagcg |
| | | guacaccuccaccaaggaggugcuggacgccacccugauccaccaguccaucaccggccuguacgagacccggaucgaccugucccagcug |
| | | ggcggcgacGGCgccCCCgagUCCgccACCgagUCCggcGGCaccUCCaccGAGuccGAGggcUCCgccGGCaccUCCaccGAGuccGAGg |
| | | gcuccGCCggcUCCgccGGCuccaccUCCaccGAGgagGGCaccUCCaccGAGuccGAGggcuccGCCggcACCuccACCgaguccgagGG |
| | | CuccGCCggcACCuccGAGuccgccACCgagUCCggcGGCaccUCCaccGAGuccGAGggcUCCuccUCCggugccgccuucaagcccaac |
| | | cccaucaacuacauccugggccuggacaucggcaucgccuccgugggcugggccaugguggagaucgacgaggaggagaaccccauccggc |
| | | ugaucgaccugggcgugcggguguucgagcgggccgaggugcccaagaccggcgacucccuggccauggcccggcggcuggcccgguccgu |
| | | gcggcggcugacccggcggcgggcccaccggcugcugcgggcccggcggcugcugaagcgggagggcgugcugcaggccgccgacuucgac |
| | | gagaacggccugaucaagucccugcccaacacccccuggcagcugcgggccgccgcccuggaccggaagcugaccccccuggagugguccg |
| | | ccgugcugcugcaccugaucaagcaccggggcuaccugucccagcggaagaacgagggcgagaccgccgacaaggagcugggcgcccugcu |
| | | gaagggcguggccaacaacgcccacgcccugcagaccggcgacuuccggacccccgccgagcuggcccugaacaaguucgagaaggagucc |
| | | ggccacauccggaaccagcggggcgacuacucccacaccuucucccggaaggaccugcaggccgagcugauccugcuguucgagaagcaga |
| | | aggaguucggcaacccccacguguccggcggccugaaggagggcaucgagacccugcugaugacccagcggcccgcccuguccggcgacgc |
| | | cgugcagaagaugcugggccacugcaccuucgagcccgccgagcccaaggccgccaagaacaccuacaccgccgagcgguucaucuggcug |
| | | accaagcugaacaaccugcggauccuggagcagggcuccgagcggccccugaccgacaccgagcgggccacccugauggacgagcccuacc |
| | | ggaaguccaagcugaccuacgcccaggcccggaagcugcugggccuggaggacaccgccuucuucaagggccugcgguacggcaaggacaa |
| | | cgccgaggccuccacccugauggagaugaaggccuaccacgccaucucccgggcccuggagaaggagggccugaaggacaagaaguccccc |
| | | cugaaccuguccuccgagcugcaggacgagaucggcaccgccuucucccuguucaagaccgacgaggacaucaccggccggcugaaggacc |
| | | gggugcagcccgagauccuggaggcccugcugaagcacaucuccuucgacaaguucgugcagaucucccugaaggcccugcggcggaucgu |
| | | gccccugauggagcagggcaagcgguacgacgaggccugcgccgagaucuacggcgaccacuacggcaagaagaacaccgaggagaagauc |
| | | uaccugccccccauccccgccgacgagauccggaaccccguggugcugcgggcccugucccaggcccggaaggugaucaacggcguggugc |
| | | ggcgguacggcucccccgcccggauccacaucgagaccgcccgggaggugggcaaguccuucaaggaccggaaggagaucgagaagcggca |
| | | ggaggagaaccggaaggaccgggagaaggccgccgccaaguuccgggaguacuuccccaacuucgugggcgagcccaaguccaaggacauc |
| | | cugaagcugcggcuguacgagcagcagcacggcaagugccuguacuccggcaaggagaucaaccuggugcggcugaacgagaagggcuacg |
| | | uggagaucgaccacgcccugcccuucucccggaccugggacgacuccuucaacaacaaggugcuggugcugggcuccgagaaccagaacaa |
| | | gggcaaccagacccccuacgaguacuucaacggcaaggacaacucccgggaguggcaggaguucaaggcccggguggagaccucccgguuc |
| | | ccccgguccaagaagcagcggauccugcugcagaaguucgacgaggacggcuucaaggagugcaaccugaacgacacccgguacgugaacc |
| | | gcuuccugugccaguucguggccgaccacauccugcugaccggcaagggcaagcggcggguguucgccuccaacggccagaucaccaaccu |
| | | gcugcggggcuucuggggccugcggaaggugcgggccgagaacgaccggcaccacgcccuggacgccguggugguggccugcuccaccgug |
| | | gccaugcagcagaagaucacccgguucgugcgguacaaggagaugaacgccuucgacggcaagaccaucgacaaggagaccggcaaggugc |
| | | ugcaccagaagacccacuucccccagcccugggaguucuucgcccaggaggugaugauccggguguucggcaagcccgacggcaagcccga |
| | | guucgaggaggccgacacccccgagaagcugcggacccugcuggccgagaagcuguccucccggcccgaggccgugcacgaguacgugacc |
| | | ccccuguucgugucccgggcccccaaccggaagauguccggcgcccacaaggacacccugcgguccgccaagcgguucgugaagcacaacg |
| | | agaagaucuccgugaagcggguguggcugaccgagaucaagcuggccgaccuggagaacauggugaacuacaagaacggccgggagaucga |
| | | gcuguacgaggcccugaaggcccggcuggaggccuacggcggcaacgccaagcaggccuucgaccccaaggacaaccccuucuacaagaag |
| | | ggcggccagcuggugaaggccgugcggguggagaagacccaggaguccggcgugcugcugaacaagaagaacgccuacaccaucgccgaca |
| | | acggcgacauggugcggguggacguguucugcaagguggacaagaagggcaagaaccaguacuucaucgugcccaucuacgccuggcaggu |
| | | ggccgagaacauccugcccgacaucgacugcaagggcuaccggaucgacgacuccuacaccuucugcuucucccugcacaaguacgaccug |
| | | aucgccuuccagaaggacgagaaguccaagguggaguucgccuacuacaucaacugcgacuccuccaacggccgguucuaccuggccuggc |
| | | acgacaagggcuccaaggagcagcaguuccggaucuccacccagaaccuggugcugauccagaaguaccaggugaacgagcugggcaagga |
| | | gauccggcccugccggcugaagaagcggccccccgugcgguccggaaagcggaccgccgacggcuccgaguucgagucccccaagaagaag |
| | | cggaagguggagAGCGAAAGCGCAACACCGGAAAGCGUCAGCGGAUGGAGACUGUUCAAGAAGAUCAGCuag |
| 5 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion | KLFIQLVQTYNQLFEENPINASGVDAKAILSARLSKSRRLENLIAQLPGEKKNGLFGNLIALSLGLTPNFKSNFDLAEDAKLQLSKDTYDD |
| | (mRNA A | DLDNLLAQIGDQYADLFLAAKNLSDAILLSDILRVNTEITKAPLSASMIKRYDEHHQDLTLLKALVRQQLPEKYKEIFFDQSKNGYAGYID |
| | with | GGASQEEFYKFIKPILEKMDGTEELLVKLNREDLLRKQRTFDNGSIPHQIHLGELHAILRRQEDFYPFLKDNREKIEKILTFRIPYYVGPL |
| | HiBit | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | tag)- | AIVDLLFKTNRKVTVKQLKEDYFKKIECFDSVEISGVEDRFNASLGTYHDLLKIIKDKDFLDNEENEDILEDIVLTLTLFEDREMIEERLK |
| | amino | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | acid | GSPAIKKGILQTVKVVDELVKVMGRHKPENIVIEMARENQTTQKGQKNSRERMKRIEEGIKELGSQILKEHPVENTQLQNEKLYLYYLQNG |
| | sequence | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | PINYILGLDIGIASVGWAMVEIDEEENPIRLIDLGVRVFERAEVPKTGDSLAMARRLARSVRRLTRRRAHRLLRARRLLKREGVLQAADFD |
| | | ENGLIKSLPNTPWQLRAAALDRKLTPLEWSAVLLHLIKHRGYLSQRKNEGETADKELGALLKGVANNAHALQTGDFRTPAELALNKFEKES |
| | | GHIRNQRGDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLVRLNEKGYVEIDHALPFSRTWDDSFNNKVLVLGSENQNKGNQTPYEYFNGKDNSREWQEFKARVETSRF |
| | | PRSKKQRILLQKFDEDGFKECNLNDTRYVNRFLCQFVADHILLTGKGKRRVFASNGQITNLLRGFWGLRKVRAENDRHHALDAVVVACSTV |
| | | AMQQKITRFVRYKEMNAFDGKTIDKETGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEALKARLEAYGGNAKQAFDPKDNPFYKK |
| | | GGQLVKAVRVEKTQESGVLLNKKNAYTIADNGDMVRVDVFCKVDKKGKNQYFIVPIYAWQVAENILPDIDCKGYRIDDSYTFCFSLHKYDL |
| | | IAFQKDEKSKVEFAYYINCDSSNGRFYLAWHDKGSKEQQFRISTQNLVLIQKYQVNELGKEIRPCRLKKRPPVRSGKRTADGSEFESPKKK |
| | | RKVESESATPESVSGWRLFKKIS* |
| 6 | Cas9- | augGACGGCUCCGGCGGCGGCUCCCCCAAGAAGAAGCGGAAGGUGGGCGGCUCCGGCGGCGGCgccgccuucaagcccaaccccaucaacu |
| | Cas9 | acauccugggccuggacaucggcaucgccuccgugggcugggccaugguggagaucgacgaggaggagaaccccauccggcugaucgaccu |
| | fusion | gggcgugcggguguucgagcgggccgaggugcccaagaccggcgacucccuggccauggcccggcggcuggcccgguccgugcggcggcug |
| | (mRNA B)- | acccggcggcgggcccaccggcugcugcgggcccggcggcugcugaagcgggagggcgugcugcaggccgccgacuucgacgagaacggcc |
| | ORF | ugaucaagucccugcccaacacccccuggcagcugcgggccgccgcccuggaccggaagcugaccccccuggagugguccgccgugcugcu |
| | sequence | gcaccugaucaagcaccggggcuaccugucccagcggaagaacgagggcgagaccgccgacaaggagcugggcgcccugcugaagggcgug |
| | | gccaacaacgcccacgcccugcagaccggcgacuuccggacccccgccgagcuggcccugaacaaguucgagaaggaguccggccacaucc |
| | | ggaaccagcggggcgacuacucccacaccuucucccggaaggaccugcaggccgagcugauccugcuguucgagaagcagaaggaguucgg |
| | | caacccccacguguccggcggccugaaggagggcaucgagacccugcugaugacccagcggcccgcccuguccggcgacgccgugcagaag |
| | | augcugggccacugcaccuucgagcccgccgagcccaaggccgccaagaacaccuacaccgccgagcgguucaucuggcugaccaagcuga |
| | | acaaccugcggauccuggagcagggcuccgagcggccccugaccgacaccgagcgggccacccugauggacgagcccuaccggaaguccaa |
| | | gcugaccuacgcccaggcccggaagcugcugggccuggaggacaccgccuucuucaagggccugcgguacggcaaggacaacgccgaggcc |
| | | uccacccugauggagaugaaggccuaccacgccaucucccgggcccuggagaaggagggccugaaggacaagaaguccccccugaaccugu |
| | | ccuccgagcugcaggacgagaucggcaccgccuucucccuguucaagaccgacgaggacaucaccggccggcugaaggaccgggugcagcc |
| | | cgagauccugcgaggcccugcugaagcacaucuccuucgacaaguucgugcagaucucccugaaggcccugggcggaucgugccccugaug |
| | | gagcagggcaagcgguacgacgaggccugcgccgagaucuacggcgaccacuacggcaagaagaacaccgaggagaagaucuaccugcccc |
| | | ccauccccgccgacgagauccggaaccccguggugcugcgggcccugucccaggcccggaaggugaucaacggcguggugcggcgguacgg |
| | | cucccccgcccggauccacaucgagaccgcccgggaggugggcaaguccuucaaggaccggaaggagaucgagaagcggcaggaggagaac |
| | | cggaaggaccgggagaaggccgccgccaaguuccgggaguacuuccccaacuucgugggcgagcccaaguccaaggacauccugaagcugc |
| | | ggcuguacgagcagcagcacggcaagugccuguacuccggcaaggagaucaaccuggugcggcugaacgagaagggcuacguggagaucga |
| | | ccacgcccugcccuucucccggaccugggacgacuccuucaacaacaaggugcuggugcugggcuccgagaaccagaacaagggcaaccag |
| | | acccccuacgaguacuucaacggcaaggacaacucccgggaguggcaggaguucaaggcccggguggagaccucccgguucccccggucca |
| | | agaagcagcggauccugcugcagaaguucgacgaggacggcuucaaggagugcaaccugaacgacacccgguacgugaaccgguuccugug |
| | | ccaguucguggccgaccacauccugcugaccggcaagggcaagcggggguguucgccuccaacggccagaucaccaaccugcugcgggcgc |
| | | uucuggggccugcggaaggugcgggccgagaacgaccggcaccacgcccuggacgccguggugguggccugcuccaccguggccaugcagc |
| | | agaagaucacccgguucgugcgguacaaggagaugaacgccuucgacggcaagaccaucgacaaggagaccggcaaggugcugcaccagaa |
| | | gacccacuucccccagcccugggaguucuucgcccaggaggugaugauccggguguucggcaagcccgacggcaagcccgaguucgaggag |
| | | gccgacacccccgagaagcugcggacccugcuggccgagaagcuguccucccggcccgaggccgugcacgaguacgugaccccccuguucg |
| | | ugucccgggcccccaaccggaagauguccggcgcccacaaggacacccugcgguccgccaagcgguucgugaagcacaacgagaagaucuc |
| | | cgugaagcggguguggcugaccgagaucaagcuggccgaccuggagaacauggugaacuacaagaacggccgggagaucgagcuguacgag |
| | | gcccugaaggcccggcuggaggccuacggcggcaacgccaagcaggccuucgaccccaaggacaaccccuucuacaagaagggcggccagc |
| | | uggugaaggccgugcggguggagaagacccaggaguccggcgugcugcugaacaagaagaacgccuacaccaucgccgacaacggcgacau |
| | | ggugcggguggacguguucugcaagguggacaagaagggcaagaaccaguacuucaucgugcccaucuacgccuggcagguggccgagaac |
| | | auccugcccgacaucgacugcaagggcuaccggaucgacgacuccuacaccuucugcuucucccugcacaaguacgaccugaucgccuucc |
| | | agaaggacgagaaguccaagguggaguucgccuacuacaucaacugcgacuccuccaacggccgguucuaccuggccuggcacgacaaggg |
| | | cuccaaggagcagcaguuccggaucuccacccagaaccuggugcugauccagaaguaccaggugaacgagcugggcaaggagauccggccc |
| | | ugccggcugaagaagcggccccccgugcggGGCGCCCCCGAGUCCGCCACCGAGUCCGGCGGCACCUCCACCGAGUCCGAGGGCUCCGCCG |
| | | GCACCUCCACCGAGUCCGAGGGCUCCGCCGGCUCCGCCGGCUCCACCUCCGGCUCCUCCUCCGGCGACAAGAAGUACUCCAUCGGCCUGGA |
| | | CAUCGGCACCAACUCCGUGGGCUGGGCCGUGAUCACCGACGAGUACAAGGUGCCCUCCAAGAAGUUCAAGGUGCUGGGCAACACCGACCGG |
| | | CACUCCAUCAAGAAGAACCUGAUCGGCGCCCUGCUGUUCGACUCCGGCGAGACCGCCGAGGCCACCCGGCUGAAGCGGACCGCCCGGCGGC |
| | | GGUACACCCGGCGGAAGAACCGGAUCUGCUACCUGCAGGAGAUCUUCUCCAACGAGAUGGCCAAGGUGGACGACUCCUUCUUCCACCGGCU |
| | | GGAGGAGUCCUUCCUGGUGGAGGAGGACAAGAAGCACGAGCGGCACCCCAUCUUCGGCAACAUCGUGGACGAGGUGGCCUACCACGAGAAG |
| | | UACCCCACCAUCUACCACCUGCGGAAGAAGCUGGUGGACUCCACCGACAAGGCCGACCUGCGGCUGAUCUACCUGGCCCUGGCCCACAUGA |
| | | UCAAGUUCCGGGGCCACUUCCUGAUCGAGGGCGACCUGAACCCCGACAACUCCGACGUGGACAAGCUGUUCAUCCAGCUGGUGCAGACCUA |
| | | CAACCAGCUGUUCGAGGAGAACCCCAUCAACGCCUCCGGCGUGGACGCCAAGGCCAUCCUGUCCGCCCGGCUGUCCAAGUCCCGGCGGCUG |
| | | GAGAACCUGAUCGCCCAGCUGCCCGGCGAGAAGAAGAACGGCCUGUUCGGCAACCUGAUCGCCCUGUCCCUGGGCCUGACCCCCAACUUCA |
| | | AGUCCAACUUCGACCUGGCCGAGGACGCCAAGCUGCAGCUGUCCAAGGACACCUACGACGACGACCUGGACAACCUGCUGGCCCAGAUCGG |
| | | CGACCAGUACGCCGACCUGUUCCUGGCCGCCAAGAACCUGUCCGACGCCAUCCUGCUGUCCGACAUCCUGCGGGUGAACACCGAGAUCACC |
| | | AAGGCCCCCCUGUCCGCCUCCAUGAUCAAGCGGUACGACGAGCACCACCAGGACCUGACCCUGCUGAAGGCCCUGGUGCGGCAGCAGCUGC |
| | | CCGAGAAGUACAAGGAGAUCUUCUUCGACCAGUCCAAGAACGGCUACGCCGGCUACAUCGACGGCGGCGCCUCCCAGGAGGAGUUCUACAA |
| | | GUUCAUCAAGCCCAUCCUGGAGAAGAUGGACGGCACCGAGGAGCUGCUGGUGAAGCUGAACCGGGAGGACCUGCUGCGGAAGCAGCGGACC |
| | | UUCGACAACGGCUCCAUCCCCCACCAGAUCCACCUGGGCGAGCUGCACGCCAUCCUGCGGCGGCAGGAGGACUUCUACCCCUUCCUGAAGG |
| | | ACAACCGGGAGAAGAUCGAGAAGAUCCUGACCUUCCGGAUCCCCUACUACGUGGGCCCCCUGGCCCGGGGCAACUCCCGGUUCGCCUGGAU |
| | | GACCCGGAAGUCCGAGGAGACCAUCACCCCCUGGAACUUCGAGGAGGUGGUGGACAAGGGCGCCUCCGCCCAGUCCUUCAUCGAGCGGAUG |
| | | ACCAACUUCGACAAGAACCUGCCCAACGAGAAGGUGCUGCCCAAGCACUCCCUGCUGUACGAGUACUUCACCGUGUACAACGAGCUGACCA |
| | | AGgugaaguacgugaccgagggcaugcggaagcccgccuuccuguccggcgagcagaagaaggccaucguggaccugcuguucaagaccaa |
| | | ccggaaggugaccgugaagcagcugaaggaggacuacuucaagaagaucgagugcuucgacuccguggagaucuccggcguggaggaccgg |
| | | uucaacgccucccugggcaccuaccacgaccugcugaagaucaucaaggacaaggacuuccuggacaacgaggagaacgaggacauccugg |
| | | aggacaucgugcugacccugacccuguucgaggaccgggagaugaucgaggagcggcugaagaccuacgcccaccuguucgacgacaaggu |
| | | gaugaagcagcugaagcggcggcgguacaccggcuggggccggcugucccggaagcugaucaacggcauccgggacaagcaguccggcaag |
| | | accauccuggacuuccugaaguccgacggcuucgccaaccggaacuucaugcagcugauccacgacgacucccugaccuucaaggaggaca |
| | | uccagaaggcccagguguccggccagggcgacucccugcacgagcacaucgccaaccuggccggcucccccgccaucaagaagggcauccu |
| | | gcagaccgugaaggugguggacgagcuggugaaggugaugggccggcacaagcccgagaacaucgugaucgagauggcccgggagaaccag |
| | | accacccagaagggccagaagaacucccgggagcggaugaagcggaucgaggagggcaucaaggagcugggcucccagauccugaaggagc |
| | | accccguggagaacacccagcugcagaacgagaagcuguaccuguacuaccugcagaacggccgggacauguacguggaccaggagcugga |
| | | caucaaccggcuguccgacuacgacguggaccacaucgugccccaguccuuccugaaggacgacuccaucgacaacaaggugcugacccgg |
| | | uccgacaagaaccggggcaaguccgacaacgugcccuccgaggagguggugaagaagaugaagaacuacuggcggcagcugcugaacgcca |
| | | agcugaucacccagcggaaguucgacaaccugaccaaggccgagcggggcggccuguccgagcuggacaaggccggcuucaucaagcggca |
| | | gcugguggagacccggcagaucaccaagcacguggcccagauccuggacucccggaugaacaccaaguacgacgagaacgacaagcugauc |
| | | cgggaggugaaggugaucacccugaaguccaagcugguguccgacuuccggaaggacuuccaguucuacaagguggcggagaucaacaacu |
| | | accaccacgcccacgacgccuaccugaacgccguggugggcaccgcccugaucaagaaguaccccaagcuggaguccgaguucguguacgg |
| | | cgacuacaagguguacgacgugcggaagaugaucgccaaguccgagcaggagaucggcaaggccaccgccaaguacuucuucuacuccaac |
| | | aucaugaacuucuucaagaccgagaucacccuggccaacggcgagauccggaagcggccccugaucgagaccaacggcgagaccggcgaga |
| | | ucgugugggacaagggccgggacuucgccaccgugcggaaggugcuguccaugccccaggugaacaucgugaagaagaccgaggugcagac |
| | | cggcggcuucuccaaggaguccauccugcccaagcggaacuccgacaagcugaucgcccggaagaaggacugggaccccaagaaguacggc |
| | | ggcuucgacucccccaccguggccuacuccgugcuggugguggccaagguggagaagggcaaguccaagaagcugaaguccgugaaggagc |
| | | ugcugggcaucaccaucauggagcgguccuccuucgagaagaaccccaucgacuuccuggaggccaagggcuacaaggaggugaagaagga |
| | | ccugaucaucaagcugcccaaguacucccuguucgagcuggagaacggccggaagcggaugcuggccuccgccggcgagcugcagaagggc |
| | | aacgagcuggcccugcccuccaaguacgugaacuuccuguaccuggccucccacuacgagaagcugaagggcucccccgaggacaacgagc |
| | | agaagcagcuguucguggagcagcacaagcacuaccuggacgagaucaucgagcagaucuccgaguucuccaagcgggugauccuggccga |
| | | cgccaaccuggacaaggugcuguccgccuacaacaagcaccgggacaagcccauccgggagcaggccgagaacaucauccaccuguucacc |
| | | cugaccaaccugggcgcccccgccgccuucaaguacuucgacaccaccaucgacAAGaagcgguacaccuccaccaaggaggugcuggacg |
| | | ccacccugauccaccaguccaucaccggccuguacgagacccggaucgaccugucccagcugggcggcgacGGCGGCGGCUCCCCCAAGAA |
| | | GAAGCGGAAGGUGuag |
| 7 | Cas9- | MDGSGGGSPKKKRKVGGSGGGAAFKPNPINYILGLDIGIASVGWAMVEIDEEENPIRLIDLGVRVFERAEVPKTGDSLAMARRLARSVRRL |
| | Cas9 | TRRRAHRLLRARRLLKREGVLQAADFDENGLIKSLPNTPWQLRAAALDRKLTPLEWSAVLLHLIKHRGYLSQRKNEGETADKELGALLKGV |
| | fusion | ANNAHALQTGDFRTPAELALNKFEKESGHIRNQRGDYSHTFSRKDLQAELILLFEKQKEFGNPHVSGGLKEGIETLLMTQRPALSGDAVQK |
| | (mRNA B)- | MLGHCTFEPAEPKAAKNTYTAERFIWLTKLNNLRILEQGSERPLTDTERATLMDEPYRKSKLTYAQARKLLGLEDTAFFKGLRYGKDNAEA |
| | amino | STLMEMKAYHAISRALEKEGLKDKKSPLNLSSELQDEIGTAFSLFKTDEDITGRLKDRVQPEILEALLKHISFDKFVQISLKALRRIVPLM |
| | acid | EQGKRYDEACAEIYGDHYGKKNTEEKIYLPPIPADEIRNPVVLRALSQARKVINGVVRRYGSPARIHIETAREVGKSFKDRKEIEKRQEEN |
| | sequence | RKDREKAAAKFREYFPNFVGEPKSKDILKLRLYEQQHGKCLYSGKEINLVRLNEKGYVEIDHALPFSRTWDDSFNNKVLVLGSENQNKGNQ |
| | | TPYEYFNGKDNSREWQEFKARVETSRFPRSKKQRILLQKFDEDGFKECNLNDTRYVNRFLCQFVADHILLTGKGKRRVFASNGQITNLLRG |
| | | FWGLRKVRAENDRHHALDAVVVACSTVAMQQKITRFVRYKEMNAFDGKTIDKETGKVLHQKTHFPQPWEFFAQEVMIRVFGKPDGKPEFEE |
| | | ADTPEKLRTLLAEKLSSRPEAVHEYVTPLFVSRAPNRKMSGAHKDTLRSAKRFVKHNEKISVKRVWLTEIKLADLENMVNYKNGREIELYE |
| | | ALKARLEAYGGNAKQAFDPKDNPFYKKGGQLVKAVRVEKTQESGVLLNKKNAYTIADNGDMVRVDVFCKVDKKGKNQYFIVPIYAWQVAEN |
| | | ILPDIDCKGYRIDDSYTFCFSLHKYDLIAFQKDEKSKVEFAYYINCDSSNGRFYLAWHDKGSKEQQFRISTQNLVLIQKYQVNELGKEIRP |
| | | CRLKKRPPVRGAPESATESGGTSTESEGSAGTSTESEGSAGSAGSTSGSSSGDKKYSIGLDIGTNSVGWAVITDEYKVPSKKFKVLGNTDR |
| | | HSIKKNLIGALLFDSGETAEATRLKRTARRRYTRRKNRICYLQEIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNSDVDKLFIQLVQTYNQLFEENPINASGVDAKAILSARLSKSRRL |
| | | ENLIAQLPGEKKNGLFGNLIALSLGLTPNFKSNFDLAEDAKLQLSKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGYAGYIDGGASQEEFYKFIKPILEKMDGTEELLVKLNREDLLRKQRT |
| | | FDNGSIPHQIHLGELHAILRRQEDFYPFLKDNREKIEKILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIVDLLFKTNRKVTVKQLKEDYFKKIECFDSVEISGVEDR |
| | | FNASLGTYHDLLKIIKDKDFLDNEENEDILEDIVLTLTLFEDREMIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIANLAGSPAIKKGILQTVKVVDELVKVMGRHKPENIVIEMARENQ |
| | | TTQKGQKNSRERMKRIEEGIKELGSQILKEHPVENTQLQNEKLYLYYLQNGRDMYVDQELDINRLSDYDVDHIVPQSFLKDDSIDNKVLTR |
| | | SDKNRGKSDNVPSEEVVKKMKNYWRQLLNAKLITQRKFDNLTKAERGGLSELDKAGFIKRQLVETRQITKHVAQILDSRMNTKYDENDKLI |
| | | REVKVITLKSKLVSDFRKDFQFYKVREINNYHHAHDAYLNAVVGTALIKKYPKLESEFVYGDYKVYDVRKMIAKSEQEIGKATAKYFFYSN |
| | | IMNFFKTEITLANGEIRKRPLIETNGETGEIVWDKGRDFATVRKVLSMPQVNIVKKTEVQTGGFSKESILPKRNSDKLIARKKDWDPKKYG |
| | | GFDSPTVAYSVLVVAKVEKGKSKKLKSVKELLGITIMERSSFEKNPIDFLEAKGYKEVKKDLIIKLPKYSLFELENGRKRMLASAGELQKG |
| | | NELALPSKYVNFLYLASHYEKLKGSPEDNEQKQLFVEQHKHYLDEIIEQISEFSKRVILADANLDKVLSAYNKHRDKPIREQAENIIHLFT |
| | | LTNLGAPAAFKYFDTTIDKKRYTSTKEVLDATLIHQSITGLYETRIDLSQLGGDGGGSPKKKRKV* |
| 8 | Cas9- | GGGAAGCUCAGAAUAAACGCUCAACUUUGGCCGGAUCUGCCACCAUGGACGGCUCCGGCGGCGGCUCCCCCAAGAAGAAGCGGAAGGUGGG |
| | Cas9 | CGGCUCCGGCGGCGGCGCCGCCUUCAAGCCCAACCCCAUCAACUACAUCCUGGGCCUGGACAUCGGCAUCGCCUCCGUGGGCUGGGCCAUG |
| | fusion | GUGGAGAUCGACGAGGAGGAGAACCCCAUCCGGCUGAUCGACCUGGGCGUGCGGGUGUUCGAGCGGGCCGAGGUGCCCAAGACCGGCGACU |
| | (mRNA B | CCCUGGCCAUGGCCCGGCGGCUGGCCCGGUCCGUGCGGCGGCUGACCCGGCGGCGGGCCCACCGGCUGCUGCGGGCCCGGCGGCUGCUGAA |
| | with | GCGGGAGGGCGUGCUGCAGGCCGCCGACUUCGACGAGAACGGCCUGAUCAAGUCCCUGCCCAACACCCCCUGGCAGCUGCGGGCCGCCGCC |
| | HiBit | CUGGACCGGAAGCUGACCCCCCUGGAGUGGUCCGCCGUGCUGCUGCACCUGAUCAAGCACCGGGGCUACCUGUCCCAGCGGAAGAACGAGG |
| | tag)- | GCGAGACCGCCGACAAGGAGCUGGGCGCCCUGCUGAAGGGCGUGGCCAACAACGCCCACGCCCUGCAGACCGGCGACUUCCGGACCCCCGC |
| | mRNA | CGAGCUGGCCCUGAACAAGUUCGAGAAGGAGUCCGGCCACAUCCGGAACCAGCGGGGCGACUACUCCCACACCUUCUCCCGGAAGGACCUG |
| | sequence | CAGGCCGAGCUGAUCCUGCUGUUCGAGAAGCAGAAGGAGUUCGGCAACCCCCACGUGUCCGGCGGCCUGAAGGAGGGCAUCGAGACCCUGC |
| | | UGAUGACCCAGCGGCCCGCCCUGUCCGGCGACGCCGUGCAGAAGAUGCUGGGCCACUGCACCUUCGAGCCCGCCGAGCCCAAGGCCGCCAA |
| | | GAACACCUACACCGCCGAGCGGUUCAUCUGGCUGACCAAGCUGAACAACCUGCGGAUCCUGGAGCAGGGCUCCGAGCGGCCCCUGACCGAC |
| | | ACCGAGCGGGCCACCCUGAUGGACGAGCCCUACCGGAAGUCCAAGCUGACCUACGCCCAGGCCCGGAAGCUGCUGGGCCUGGAGGACACCG |
| | | CCUUCUUCAAGGGCCUGCGGUACGGCAAGGACAACGCCGAGGCCUCCACCCUGAUGGAGAUGAAGGCCUACCACGCCAUCUCCCGGGCCCU |
| | | GGAGAAGGAGGGCCUGAAGGACAAGAAGUCCCCCCUGAACCUGUCCUCCGAGCUGCAGGACGAGAUCGGCACCGCCUUCUCCCUGUUCAAG |
| | | ACCGACGAGGACAUCACCGGCCGGCUGAAGGACCGGGUGCAGCCCGAGAUCCUGGAGGCCCUGCUGAAGCACAUCUCCUUCGACAAGUUCG |
| | | UGCAGAUCUCCCUGAAGGCCCUGCGGCGGAUCGUGCCCCUGAUGGAGCAGGGCAAGCGGUACGACGAGGCCUGCGCCGAGAUCUACGGCGA |
| | | CCACUACGGCAAGAAGAACACCGAGGAGAAGAUCUACCUGCCCCCCAUCCCCGCCGACGAGAUCCGGAACCCCGUGGUGCUGCGGGCCCUG |
| | | UCCCAGGCCCGGAAGGUGAUCAACGGCGUGGUGCGGCGGUACGGCUCCCCCGCCCGGAUCCACAUCGAGACCGCCCGGGAGGUGGGCAAGU |
| | | CCUUCAAGGACCGGAAGGAGAUCGAGAAGCGGCAGGAGGAGAACCGGAAGGACCGGGAGAAGGCCGCCGCCAAGUUCCGGGAGUACUUCCC |
| | | CAACUUCGUGGGCGAGCCCAAGUCCAAGGACAUCCUGAAGCUGCGGCUGUACGAGCAGCAGCACGGCAAGUGCCUGUACUCCGGCAAGGAG |
| | | AUCAACCUGGUGCGGCUGAACGAGAAGGGCUACGUGGAGAUCGACCACGCCCUGCCCUUCUCCCGGACCUGGGACGACUCCUUCAACAACA |
| | | AGGUGCUGGUGCUGGGCUCCGAGAACCAGAACAAGGGCAACCAGACCCCCUACGAGUACUUCAACGGCAAGGACAACUCCCGGGAGUGGCA |
| | | GGAGUUCAAGGCCCGGGUGGAGACCUCCCGGUUCCCCCGGUCCAAGAAGCAGCGGAUCCUGCUGCAGAAGUUCGACGAGGACGGCUUCAAG |
| | | GAGUGCAACCUGAACGACACCCGGUACGUGAACCGGUUCCUGUGCCAGUUCGUGGCCGACCACAUCCUGCUGACCGGCAAGGGCAAGCGGC |
| | | GGGUGUUCGCCUCCAACGGCCAGAUCACCAACCUGCUGCGGGGCUUCUGGGGCCUGCGGAAGGUGCGGGCCGAGAACGACCGGCACCACGC |
| | | CCUGGACGCCGUGGUGGUGGCCUGCUCCACCGUGGCCAUGCAGCAGAAGAUCACCCGGUUCGUGCGGUACAAGGAGAUGAACGCCUUCGAC |
| | | GGCAAGACCAUCGACAAGGAGACCGGCAAGGUGCUGCACCAGAAGACCCACUUCCCCCAGCCCUGGGAGUUCUUCGCCCAGGAGGUGAUGA |
| | | UCCGGGUGUUCGGCAAGCCCGACGGCAAGCCCGAGUUCGAGGAGGCCGACACCCCCGAGAAGCUGCGGACCCUGCUGGCCGAGAAGCUGUC |
| | | CUCCCGGCCCGAGGCCGUGCACGAGUACGUGACCCCCCUGUUCGUGUCCCGGGCCCCCAACCGGAAGAUGUCCGGCGCCCACAAGGACACC |
| | | CUGCGGUCCGCCAAGCGGUUCGUGAAGCACAACGAGAAGAUCUCCGUGAAGCGGGUGUGGCUGACCGAGAUCAAGCUGGCCGACCUGGAGA |
| | | ACAUGGUGAACUACAAGAACGGCCGGGAGAUCGAGCUGUACGAGGCCCUGAAGGCCCGGCUGGAGGCCUACGGCGGCAACGCCAAGCAGGC |
| | | CUUCGACCCCAAGGACAACCCCUUCUACAAGAAGGGCGGCCAGCUGGUGAAGGCCGUGCGGGUGGAGAAGACCCAGGAGUCCGGCGUGCUG |
| | | CUGAACAAGAAGAACGCCUACACCAUCGCCGACAACGGCGACAUGGUGCGGGUGGACGUGUUCUGCAAGGUGGACAAGAAGGGCAAGAACC |
| | | AGUACUUCAUCGUGCCCAUCUACGCCUGGCAGGUGGCCGAGAACAUCCUGCCCGACAUCGACUGCAAGGGCUACCGGAUCGACGACUCCUA |
| | | CACCUUCUGCUUCUCCCUGCACAAGUACGACCUGAUCGCCUUCCAGAAGGACGAGAAGUCCAAGGUGGAGUUCGCCUACUACAUCAACUGC |
| | | GACUCCUCCAACGGCCGGUUCUACCUGGCCUGGCACGACAAGGGCUCCAAGGAGCAGCAGUUCCGGAUCUCCACCCAGAACCUGGUGCUGA |
| | | UCCAGAAGUACCAGGUGAACGAGCUGGGCAAGGAGAUCCGGCCCUGCCGGCUGAAGAAGCGGCCCCCCGUGCGGGGCGCCCCCGAGUCCGC |
| | | CACCGAGUCCGGCGGCACCUCCACCGAGUCCGAGGGCUCCGCCGGCACCUCCACCGAGUCCGAGGGCUCCGCCGGCUCCGCCGGCUCCACC |
| | | UCCGGCUCCUCCUCCGGCGACAAGAAGUACUCCAUCGGCCUGGACAUCGGCACCAACUCCGUGGGCUGGGCCGUGAUCACCGACGAGUACA |
| | | AGGUGCCCUCCAAGAAGUUCAAGGUGCUGGGCAACACCGACCGGCACUCCAUCAAGAAGAACCUGAUCGGCGCCCUGCUGUUCGACUCCGG |
| | | CGAGACCGCCGAGGCCACCCGGCUGAAGCGGACCGCCCGGCGGCGGUACACCCGGCGGAAGAACCGGAUCUGCUACCUGCAGGAGAUCUUC |
| | | UCCAACGAGAUGGCCAAGGUGGACGACUCCUUCUUCCACCGGCUGGAGGAGUCCUUCCUGGUGGAGGAGGACAAGAAGCACGAGCGGCACC |
| | | CCAUCUUCGGCAACAUCGUGGACGAGGUGGCCUACCACGAGAAGUACCCCACCAUCUACCACCUGCGGAAGAAGCUGGUGGACUCCACCGA |
| | | CAAGGCCGACCUGCGGCUGAUCUACCUGGCCCUGGCCCACAUGAUCAAGUUCCGGGGCCACUUCCUGAUCGAGGGCGACCUGAACCCCGAC |
| | | AACUCCGACGUGGACAAGCUGUUCAUCCAGCUGGUGCAGACCUACAACCAGCUGUUCGAGGAGAACCCCAUCAACGCCUCCGGCGUGGACG |
| | | CCAAGGCCAUCCUGUCCGCCCGGCUGUCCAAGUCCCGGCGGCUGGAGAACCUGAUCGCCCAGCUGCCCGGCGAGAAGAAGAACGGCCUGUU |
| | | CGGCAACCUGAUCGCCCUGUCCCUGGGCCUGACCCCCAACUUCAAGUCCAACUUCGACCUGGCCGAGGACGCCAAGCUGCAGCUGUCCAAG |
| | | GACACCUACGACGACGACCUGGACAACCUGCUGGCCCAGAUCGGCGACCAGUACGCCGACCUGUUCCUGGCCGCCAAGAACCUGUCCGACG |
| | | CCAUCCUGCUGUCCGACAUCCUGCGGGUGAACACCGAGAUCACCAAGGCCCCCCUGUCCGCCUCCAUGAUCAAGCGGUACGACGAGCACCA |
| | | CCAGGACCUGACCCUGCUGAAGGCCCUGGUGCGGCAGCAGCUGCCCGAGAAGUACAAGGAGAUCUUCUUCGACCAGUCCAAGAACGGCUAC |
| | | GCCGGCUACAUCGACGGCGGCGCCUCCCAGGAGGAGUUCUACAAGUUCAUCAAGCCCAUCCUGGAGAAGAUGGACGGCACCGAGGAGCUGC |
| | | UGGUGAAGCUGAACCGGGAGGACCUGCUGCGGAAGCAGCGGACCUUCGACAACGGCUCCAUCCCCCACCAGAUCCACCUGGGCGAGCUGCA |
| | | CGCCAUCCUGCGGCGGCAGGAGGACUUCUACCCCUUCCUGAAGGACAACCGGGAGAAGAUCGAGAAGAUCCUGACCUUCCGGAUCCCCUAC |
| | | UACGUGGGCCCCCUGGCCCGGGGCAACUCCCGGUUCGCCUGGAUGACCCGGAAGUCCGAGGAGACCAUCACCCCCUGGAACUUCGAGGAGG |
| | | UGGUGGACAAGGGCGCCUCCGCCCAGUCCUUCAUCGAGCGGAUGACCAACUUCGACAAGAACCUGCCCAACGAGAAGGUGCUGCCCAAGCA |
| | | CUCCCUGCUGUACGAGUACUUCACCGUGUACAACGAGCUGACCAAGGUGAAGUACGUGACCGAGGGCAUGCGGAAGCCCGCCUUCCUGUCC |
| | | GGCGAGCAGAAGAAGGCCAUCGUGGACCUGCUGUUCAAGACCAACCGGAAGGUGACCGUGAAGCAGCUGAAGGAGGACUACUUCAAGAAGA |
| | | UCGAGUGCUUCGACUCCGUGGAGAUCUCCGGCGUGGAGGACCGGUUCAACGCCUCCCUGGGCACCUACCACGACCUGCUGAAGAUCAUCAA |
| | | GGACAAGGACUUCCUGGACAACGAGGAGAACGAGGACAUCCUGGAGGACAUCGUGCUGACCCUGACCCUGUUCGAGGACCGGGAGAUGAUC |
| | | GAGGAGCGGCUGAAGACCUACGCCCACCUGUUCGACGACAAGGUGAUGAAGCAGCUGAAGCGGCGGCGGUACACCGGCUGGGGCCGGCUGU |
| | | CCCGGAAGCUGAUCAACGGCAUCCGGGACAAGCAGUCCGGCAAGACCAUCCUGGACUUCCUGAAGUCCGACGGCUUCGCCAACCGGAACUU |
| | | CAUGCAGCUGAUCCACGACGACUCCCUGACCUUCAAGGAGGACAUCCAGAAGGCCCAGGUGUCCGGCCAGGGCGACUCCCUGCACGAGCAC |
| | | AUCGCCAACCUGGCCGGCUCCCCCGCCAUCAAGAAGGGCAUCCUGCAGACCGUGAAGGUGGUGGACGAGCUGGUGAAGGUGAUGGGCCGGC |
| | | ACAAGCCCGAGAACAUCGUGAUCGAGAUGGCCCGGGAGAACCAGACCACCCAGAAGGGCCAGAAGAACUCCCGGGAGCGGAUGAAGCGGAU |
| | | CGAGGAGGGCAUCAAGGAGCUGGGCUCCCAGAUCCUGAAGGAGCACCCCGUGGAGAACACCCAGCUGCAGAACGAGAAGCUGUACCUGUAC |
| | | UACCUGCAGAACGGCCGGGACAUGUACGUGGACCAGGAGCUGGACAUCAACCGGCUGUCCGACUACGACGUGGACCACAUCGUGCCCCAGU |
| | | CCUUCCUGAAGGACGACUCCAUCGACAACAAGGUGCUGACCCGGUCCGACAAGAACCGGGGCAAGUCCGACAACGUGCCCUCCGAGGAGGU |
| | | GGUGAAGAAGAUGAAGAACUACUGGCGGCAGCUGCUGAACGCCAAGCUGAUCACCCAGCGGAAGUUCGACAACCUGACCAAGGCCGAGCGG |
| | | GGCGGCCUGUCCGAGCUGGACAAGGCCGGCUUCAUCAAGCGGCAGCUGGUGGAGACCCGGCAGAUCACCAAGCACGUGGCCCAGAUCCUGG |
| | | ACUCCCGGAUGAACACCAAGUACGACGAGAACGACAAGCUGAUCCGGGAGGUGAAGGUGAUCACCCUGAAGUCCAAGCUGGUGUCCGACUU |
| | | CCGGAAGGACUUCCAGUUCUACAAGGUGCGGGAGAUCAACAACUACCACCACGCCCACGACGCCUACCUGAACGCCGUGGUGGGCACCGCC |
| | | CUGAUCAAGAAGUACCCCAAGCUGGAGUCCGAGUUCGUGUACGGCGACUACAAGGUGUACGACGUGCGGAAGAUGAUCGCCAAGUCCGAGC |
| | | AGGAGAUCGGCAAGGCCACCGCCAAGUACUUCUUCUACUCCAACAUCAUGAACUUCUUCAAGACCGAGAUCACCCUGGCCAACGGCGAGAU |
| | | CCGGAAGCGGCCCCUGAUCGAGACCAACGGCGAGACCGGCGAGAUCGUGUGGGACAAGGGCCGGGACUUCGCCACCGUGCGGAAGGUGCUG |
| | | UCCAUGCCCCAGGUGAACAUCGUGAAGAAGACCGAGGUGCAGACCGGCGGCUUCUCCAAGGAGUCCAUCCUGCCCAAGCGGAACUCCGACA |
| | | AGCUGAUCGCCCGGAAGAAGGACUGGGACCCCAAGAAGUACGGCGGCUUCGACUCCCCCACCGUGGCCUACUCCGUGCUGGUGGUGGCCAA |
| | | GGUGGAGAAGGGCAAGUCCAAGAAGCUGAAGUCCGUGAAGGAGCUGCUGGGCAUCACCAUCAUGGAGCGGUCCUCCUUCGAGAAGAACCCC |
| | | AUCGACUUCCUGGAGGCCAAGGGCUACAAGGAGGUGAAGAAGGACCUGAUCAUCAAGCUGCCCAAGUACUCCCUGUUCGAGCUGGAGAACG |
| | | GCCGGAAGCGGAUGCUGGCCUCCGCCGGCGAGCUGCAGAAGGGCAACGAGCUGGCCCUGCCCUCCAAGUACGUGAACUUCCUGUACCUGGC |
| | | CUCCCACUACGAGAAGCUGAAGGGCUCCCCCGAGGACAACGAGCAGAAGCAGCUGUUCGUGGAGCAGCACAAGCACUACCUGGACGAGAUC |
| | | AUCGAGCAGAUCUCCGAGUUCUCCAAGCGGGUGAUCCUGGCCGACGCCAACCUGGACAAGGUGCUGUCCGCCUACAACAAGCACCGGGACA |
| | | AGCCCAUCCGGGAGCAGGCCGAGAACAUCAUCCACCUGUUCACCCUGACCAACCUGGGCGCCCCCGCCGCCUUCAAGUACUUCGACACCAC |
| | | CAUCGACAAGAAGCGGUACACCUCCACCAAGGAGGUGCUGGACGCCACCCUGAUCCACCAGUCCAUCACCGGCCUGUACGAGACCCGGAUC |
| | | GACCUGUCCCAGCUGGGCGGCGACGGCGGCGGCUCCCCCAAGAAGAAGCGGAAGGUGUCCGAGUCCGCCACCCCCGAGUCCGUGUCCGGCU |
| | | GGCGGCUGUUCAAGAAGAUCUCCUAGCUAGCACCAGCCUCAAGAACACCCGAAUGGAGUCUCUAAGCUACAUAAUACCAACUUACACUUUA |
| | | CAAAAUGUUGUCCCCCAAAAUGUAGCCAUUCGUAUCUGCUCCUAAUAAAAAGAAAGUUUCUUCACAUUCUCUCGAGAAAAAAAAAAAAUGG |
| | | AAAAAAAAAAAACGGAAAAAAAAAAAAGGUAAAAAAAAAAAAUAUAAAAAAAAAAAACAUAAAAAAAAAAAACGAAAAAAAAAAAACGUAA |
| | | AAAAAAAAAACUCAAAAAAAAAAAGAUAAAAAAAAAAAACCUAAAAAAAAAAAAUGUAAAAAAAAAAAAGGGAAAAAAAAAAAACGCAAAA |
| | | AAAAAAAACACAAAAAAAAAAAAUGCAAAAAAAAAAAAUCGAAAAAAAAAAAAUCUAAAAAAAAAAAACGAAAAAAAAAAAACCCAAAAAA |
| | | AAAAAAGACAAAAAAAAAAAAUAGAAAAAAAAAAAGUUAAAAAAAAAAAACUGAAAAAAAAAAAAUUUAAAAAAAAAAAAUCUAG |
| 9 | Cas9- | augGACGGCUCCGGCGGCGGCUCCCCCAAGAAGAAGCGGAAGGUGGGCGGCUCCGGCGGCGGCgccgccuucaagcccaaccccaucaacu |
| | Cas9 | acauccugggccuggacaucggcaucgccuccgugggcugggccaugguggagaucgacgaggaggagaaccccauccggcugaucgaccu |
| | fusion | gggcgugcggguguucgagcgggccgaggugcccaagaccggcgacucccuggccauggcccggcggcuggcccgguccgugcggcggcug |
| | (mRNA B | acccggcgggcggcccaccggcugcugcgggcccggcggcugcugaagcgggagggcgugcugcaggccgccgacuucgacgagaacggcc |
| | with | ugaucaagucccugcccaacacccccuggcagcugcgggccgccgcccuggaccggaagcugaccccccuggagugguccgccgugcugcu |
| | HiBit | gcaccugaucaagcaccggggcuaccugucccagcggaagaacgagggcgagaccgccgacaaggagcugcgggcccugcugaagggcgug |
| | tag)- | gccaacaacgcccacgcccugcagaccggcgacuuccggacccccgccgagcuggcccugaacaaguucgagaaggaguccggccacaucc |
| | ORF | ggaaccagcggggcgacuacucccacaccuucucccggaaggaccugcaggccgagcugauccugcuguucgagaagcagaaggaguucgg |
| | sequence | caacccccacguguccggcggccugaaggagggcaucgagacccugcugaugacccagcggcccgcccuguccggcgacgccgugcagaag |
| | | augcugggccacugcaccuucgagcccgccgagcccaaggccgccaagaacaccuacaccgccgagcgguucaucuggcugaccaagcuga |
| | | acaaccugcggauccuggagcagggcuccgagcggccccugaccgacaccgagcgggccacccugauggacgagcccuaccggaaguccaa |
| | | gcugaccuacgcccaggcccggaagcugcugggccuggaggacaccgccuucuucaagggccugcgguacggcaaggacaacgccgaggcc |
| | | uccacccugauggagaugaaggccuaccacgccaucucccgggcccuggagaaggagggccugaaggacaagaaguccccccugaaccugu |
| | | ccuccgagcugcaggacgagaucggcaccgccuucucccuguucaagaccgacgaggacaucaccggccggcugaaggaccgggugcagcc |
| | | cgagauccuggaggcccugcugaagcacaucuccuucgacaaguucgugcagaucucccugaaggcccugcggcggaucgugccccugaug |
| | | gagcagggcaagcgguacgacgaggccugcgccgagaucuacggcgaccacuacggcaagaagaacaccgaggagaagaucuaccugcccc |
| | | ccauccccgccgacgagauccggaaccccguggugcugcgggcccugucccaggcccggaaggugaucaacggcguggugcggcgguacgg |
| | | cucccccgcccggauccacaucgagaccgcccgggaggugggcaaguccuucaaggaccggaaggagaucgagaagcggcaggaggagaac |
| | | cggaaggaccgggagaaggccgccgccaaguuccgggaguacuuccccaacuucgugggcgagcccaaguccaaggacauccugaagcugc |
| | | ggcuguacgagcagcagcacggcaagugccuguacuccggcaaggagaucaaccuggugcggcugaacgagaagggcuacguggagaucga |
| | | ccacgcccugcccuucucccggaccugggacgacuccuucaacaacaaggugcuggugcugggcuccgagaaccagaacaagggcaaccag |
| | | acccccuacgaguacuucaacggcaaggacaacucccgggaguggcaggaguucaaggcccggguggagaccucccgguucccccggucca |
| | | agaagcagcggauccugcugcagaaguucgacgaggacggcuucaaggagugcaaccugaacgacacccgguacgugaaccgguuccugug |
| | | ccaguucguggccgaccacauccugcugaccggcaagggcaagcggggguguucgccuccaacggccagaucaccaaccugcugcgcgggc |
| | | uucuggggccugcggaaggugcgggccgagaacgaccggcaccacgcccuggacgccguggugguggccugcuccaccguggccaugcagc |
| | | agaagaucacccgguucgugcgguacaaggagaugaacgccuucgacggcaagaccaucgacaaggagaccggcaaggugcugcaccagaa |
| | | gacccacuucccccagcccugggaguucuucgcccaggaggugaugauccggguguucggcaagcccgacggcaagcccgaguucgaggag |
| | | gccgacacccccgagaagcugcggacccugcuggccgagaagcuguccucccggcccgaggccgugcacgaguacgugaccccccuguucg |
| | | ugucccgggcccccaaccggaagauguccggcgcccacaaggacacccugcgguccgccaagcgguucgugaagcacaacgagaagaucuc |
| | | cgugaagcggguguggcugaccgagaucaagcuggccgaccuggagaacauggugaacuacaagaacggccgggagaucgagcuguacgag |
| | | gcccugaaggcccggcuggaggccuacggcggcaacgccaagcaggccuucgaccccaaggacaaccccuucuacaagaagggcggccagc |
| | | uggugaaggccgugcggguggagaagacccaggaguccggcgugcugcugaacaagaagaacgccuacaccaucgccgacaacggcgacau |
| | | ggugcggguggacguguucugcaagguggacaagaagggcaagaaccaguacuucaucgugcccaucuacgccuggcagguggccgagaac |
| | | auccugcccgacaucgacugcaagcggcuaccggaucgacgacuccuacaccuucugcuucucccugcacaaguacgaccugaugccuucc |
| | | agaaggacgagaaguccaagguggaguucgccuacuacaucaacugcgacuccuccaacggccgguucuaccuggccuggcacgacaaggg |
| | | cuccaaggagcagcaguuccggaucuccacccagaaccuggugcugauccagaaguaccaggugaacgagcugggcaaggagauccggccc |
| | | ugccggcugaagaagcggccccccgugcggGGCGCCCCCGAGUCCGCCACCGAGUCCGGCGGCACCUCCACCGAGUCCGAGGGCUCCGCCG |
| | | GCACCUCCACCGAGUCCGAGGGCUCCGCCGGCUCCGCCGGCUCCACCUCCGGCUCCUCCUCCGGCGACAAGAAGUACUCCAUCGGCCUGGA |
| | | CAUCGGCACCAACUCCGUGGGCUGGGCCGUGAUCACCGACGAGUACAAGGUGCCCUCCAAGAAGUUCAAGGUGCUGGGCAACACCGACCGG |
| | | CACUCCAUCAAGAAGAACCUGAUCGGCGCCCUGCUGUUCGACUCCGGCGAGACCGCCGAGGCCACCCGGCUGAAGCGGACCGCCCGGCGGC |
| | | GGUACACCCGGCGGAAGAACCGGAUCUGCUACCUGCAGGAGAUCUUCUCCAACGAGAUGGCCAAGGUGGACGACUCCUUCUUCCACCGGCU |
| | | GGAGGAGUCCUUCCUGGUGGAGGAGGACAAGAAGCACGAGCGGCACCCCAUCUUCGGCAACAUCGUGGACGAGGUGGCCUACCACGAGAAG |
| | | UACCCCACCAUCUACCACCUGCGGAAGAAGCUGGUGGACUCCACCGACAAGGCCGACCUGCGGCUGAUCUACCUGGCCCUGGCCCACAUGA |
| | | UCAAGUUCCGGGGCCACUUCCUGAUCGAGGGCGACCUGAACCCCGACAACUCCGACGUGGACAAGCUGUUCAUCCAGCUGGUGCAGACCUA |
| | | CAACCAGCUGUUCGAGGAGAACCCCAUCAACGCCUCCGGCGUGGACGCCAAGGCCAUCCUGUCCGCCCGGCUGUCCAAGUCCCGGCGGCUG |
| | | GAGAACCUGAUCGCCCAGCUGCCCGGCGAGAAGAAGAACGGCCUGUUCGGCAACCUGAUCGCCCUGUCCCUGGGCCUGACCCCCAACUUCA |
| | | AGUCCAACUUCGACCUGGCCGAGGACGCCAAGCUGCAGCUGUCCAAGGACACCUACGACGACGACCUGGACAACCUGCUGGCCCAGAUCGG |
| | | CGACCAGUACGCCGACCUGUUCCUGGCCGCCAAGAACCUGUCCGACGCCAUCCUGCUGUCCGACAUCCUGCGGGUGAACACCGAGAUCACC |
| | | AAGGCCCCCCUGUCCGCCUCCAUGAUCAAGCGGUACGACGAGCACCACCAGGACCUGACCCUGCUGAAGGCCCUGGUGCGGCAGCAGCUGC |
| | | CCGAGAAGUACAAGGAGAUCUUCUUCGACCAGUCCAAGAACGGCUACGCCGGCUACAUCGACGGCGGCGCCUCCCAGGAGGAGUUCUACAA |
| | | GUUCAUCAAGCCCAUCCUGGAGAAGAUGGACGGCACCGAGGAGCUGCUGGUGAAGCUGAACCGGGAGGACCUGCUGCGGAAGCAGCGGACC |
| | | UUCGACAACGGCUCCAUCCCCCACCAGAUCCACCUGGGCGAGCUGCACGCCAUCCUGCGGCGGCAGGAGGACUUCUACCCCUUCCUGAAGG |
| | | ACAACCGGGAGAAGAUCGAGAAGAUCCUGACCUUCCGGAUCCCCUACUACGUGGGCCCCCUGGCCCGGGGCAACUCCCGGUUCGCCUGGAU |
| | | GACCCGGAAGUCCGAGGAGACCAUCACCCCCUGGAACUUCGAGGAGGUGGUGGACAAGGGCGCCUCCGCCCAGUCCUUCAUCGAGCGGAUG |
| | | ACCAACUUCGACAAGAACCUGCCCAACGAGAAGGUGCUGCCCAAGCACUCCCUGCUGUACGAGUACUUCACCGUGUACAACGAGCUGACCA |
| | | AGgugaaguacgugaccgagggcaugcggaagcccgccuuccuguccggcgagcagaagaaggccaucguggaccugcuguucaagaccaa |
| | | ccggaaggugaccgugaagcagcugaaggaggacuacuucaagaagaucgagugcuucgacuccguggagaucuccggcguggaggaccgg |
| | | uucaacgccucccugggcaccuaccacgaccugcugaagaucaucaaggacaaggacuuccuggacaacgaggagaacgaggacauccugg |
| | | aggacaucgugcugacccugacccuguucgaggaccgggagaugaucgaggagcggcugaagaccuacgcccaccuguucgacgacaaggu |
| | | gaugaagcagcugaagcggcggcgguacaccggcuggggccggcugucccggaagcugaucaacggcauccgggacaagcaguccggcaag |
| | | accauccuggacuuccugaaguccgacggcuucgccaaccggaacuucaugcagcugauccacgacgacucccugaccuucaaggaggaca |
| | | uccagaaggcccagguguccggccagggcgacucccugcacgagcacaucgccaaccuggccggcucccccgccaucaagaagggcauccu |
| | | gcagaccgugaaggugguggacgagcuggugaaggugaugggccggcacaagcccgagaacaucgugaucgagauggcccgggagaaccag |
| | | accacccagaagggccagaagaacucccgggagcggaugaagcggaucgaggagggcaucaaggagcugggcucccagauccugaaggagc |
| | | accccguggagaacacccagcugcagaacgagaagcuguaccuguacuaccugcagaacggccgggacauguacguggaccaggagcugga |
| | | caucaaccggcuguccgacuacgacguggaccacaucgugccccaguccuuccugaaggacgacuccaucgacaacaaggugcugacccgg |
| | | uccgacaagaaccggggcaaguccgacaacgugcccuccgaggagguggcugaagaagaugaagaacuacuggggcagcugcugaacgcca |
| | | agcugaucacccagcggaaguucgacaaccugaccaaggccgagcggggcggccuguccgagcuggacaaggccggcuucaucaagcggca |
| | | gcugguggagacccggcagaucaccaagcacguggcccagauccuggacucccggaugaacaccaaguacgacgagaacgacaagcugauc |
| | | cgggaggugaaggugaucacccugaaguccaagcugguguccgacuuccggaaggacuuccaguucuacaaggugcgggagaucaacaacu |
| | | accaccacgcccacgacgccuaccugaacgccguggugggcaccgcccugaucaagaaguaccccaagcuggaguccgaguucguguacgg |
| | | cgacuacaagguguacgacgugcggaagaugaucgccaaguccgagcaggagaucggcaaggccaccgccaaguacuucuucuacuccaac |
| | | aucaugaacuucuucaagaccgagaucacccuggccaacggcgagauccggaagcggccccugaucgagaccaacggcgagaccggcgaga |
| | | ucgugugggacaagggccgggacuucgccaccgugcggaaggugcuguccaugccccaggugaacaucgugaagaagaccgaggugcagac |
| | | cggcggcuucuccaaggaguccauccugcccaagcggaacuccgacaagcugaucgcccggaagaaggacugggaccccaagaaguacggc |
| | | ggcuucgacucccccaccguggccuacuccgugcuggugguggccaagguggagaagggcaaguccaagaagcugaaguccgugaaggagc |
| | | ugcugggcaucaccaucauggagcgguccuccuucgagaagaaccccaucgacuuccuggaggccaagggcuacaaggaggugaagaagga |
| | | ccugaucaucaagcugcccaaguacucccuguucgagcuggagaacggccggaagcggaugcuggccuccgccggcgagcugcagaagggc |
| | | aacgagcuggcccugcccuccaaguacgugaacuuccuguaccuggccucccacuacgagaagcugaagggcucccccgaggacaacgagc |
| | | agaagcagcuguucguggagcagcacaagcacuaccuggacgagaucaucgagcagaucuccgaguucuccaaggcggugauccuggccga |
| | | cgccaaccuggacaaggugcuguccgccuacaacaagcaccgggacaagcccauccgggagcaggccgagaacaucauccaccuguucacc |
| | | cugaccaaccugggcgcccccgccgccuucaaguacuucgacaccaccaucgacAAGaagcgguacaccuccaccaaggaggugcuggacg |
| | | ccacccugauccaccaguccaucaccggccuguacgagacccggaucgaccugucccagcugggggcgacGGCGGCGGCUCCCCCAAGAA |
| | | GAAGCGGAAGGUGUCCGAGUCCGCCACCCCCGAGUCCGUGUCCGGCUGGCGGCUGUUCAAGAAGAUCUCCuag |
| 10 | Cas9- | MDGSGGGSPKKKRKVGGSGGGAAFKPNPINYILGLDIGIASVGWAMVEIDEEENPIRLIDLGVRVFERAEVPKTGDSLAMARRLARSVRRL |
| | Cas9 | TRRRAHRLLRARRLLKREGVLQAADFDENGLIKSLPNTPWQLRAAALDRKLTPLEWSAVLLHLIKHRGYLSQRKNEGETADKELGALLKGV |
| | fusion | ANNAHALQTGDFRTPAELALNKFEKESGHIRNQRGDYSHTFSRKDLQAELILLFEKQKEFGNPHVSGGLKEGIETLLMTQRPALSGDAVQK |
| | (mRNA B | MLGHCTFEPAEPKAAKNTYTAERFIWLTKLNNLRILEQGSERPLTDTERATLMDEPYRKSKLTYAQARKLLGLEDTAFFKGLRYGKDNAEA |
| | with | STLMEMKAYHAISRALEKEGLKDKKSPLNLSSELQDEIGTAFSLFKTDEDITGRLKDRVQPEILEALLKHISFDKFVQISLKALRRIVPLM |
| | HiBit | EQGKRYDEACAEIYGDHYGKKNTEEKIYLPPIPADEIRNPVVLRALSQARKVINGVVRRYGSPARIHIETAREVGKSFKDRKEIEKRQEEN |
| | tag)- | RKDREKAAAKFREYFPNFVGEPKSKDILKLRLYEQQHGKCLYSGKEINLVRLNEKGYVEIDHALPFSRTWDDSFNNKVLVLGSENQNKGNQ |
| | amino | TPYEYFNGKDNSREWQEFKARVETSRFPRSKKQRILLQKFDEDGFKECNLNDTRYVNRFLCQFVADHILLTGKGKRRVFASNGQITNLLRG |
| | acid | FWGLRKVRAENDRHHALDAVVVACSTVAMQQKITRFVRYKEMNAFDGKTIDKETGKVLHQKTHFPQPWEFFAQEVMIRVFGKPDGKPEFEE |
| | sequence | ADTPEKLRTLLAEKLSSRPEAVHEYVTPLFVSRAPNRKMSGAHKDTLRSAKRFVKHNEKISVKRVWLTEIKLADLENMVNYKNGREIELYE |
| | | ALKARLEAYGGNAKQAFDPKDNPFYKKGGQLVKAVRVEKTQESGVLLNKKNAYTIADNGDMVRVDVFCKVDKKGKNQYFIVPIYAWQVAEN |
| | | ILPDIDCKGYRIDDSYTFCFSLHKYDLIAFQKDEKSKVEFAYYINCDSSNGRFYLAWHDKGSKEQQFRISTQNLVLIQKYQVNELGKEIRP |
| | | CRLKKRPPVRGAPESATESGGTSTESEGSAGTSTESEGSAGSAGSTSGSSSGDKKYSIGLDIGTNSVGWAVITDEYKVPSKKFKVLGNTDR |
| | | HSIKKNLIGALLFDSGETAEATRLKRTARRRYTRRKNRICYLQEIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNSDVDKLFIQLVQTYNQLFEENPINASGVDAKAILSARLSKSRRL |
| | | ENLIAQLPGEKKNGLFGNLIALSLGLTPNFKSNFDLAEDAKLQLSKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGYAGYIDGGASQEEFYKFIKPILEKMDGTEELLVKLNREDLLRKQRT |
| | | FDNGSIPHQIHLGELHAILRRQEDFYPFLKDNREKIEKILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIVDLLFKTNRKVTVKQLKEDYFKKIECFDSVEISGVEDR |
| | | FNASLGTYHDLLKIIKDKDFLDNEENEDILEDIVLTLTLFEDREMIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIANLAGSPAIKKGILQTVKVVDELVKVMGRHKPENIVIEMARENQ |
| | | TTQKGQKNSRERMKRIEEGIKELGSQILKEHPVENTQLQNEKLYLYYLQNGRDMYVDQELDINRLSDYDVDHIVPQSFLKDDSIDNKVLTR |
| | | SDKNRGKSDNVPSEEVVKKMKNYWRQLLNAKLITQRKFDNLTKAERGGLSELDKAGFIKRQLVETRQITKHVAQILDSRMNTKYDENDKLI |
| | | REVKVITLKSKLVSDFRKDFQFYKVREINNYHHAHDAYLNAVVGTALIKKYPKLESEFVYGDYKVYDVRKMIAKSEQEIGKATAKYFFYSN |
| | | IMNFFKTEITLANGEIRKRPLIETNGETGEIVWDKGRDFATVRKVLSMPQVNIVKKTEVQTGGFSKESILPKRNSDKLIARKKDWDPKKYG |
| | | GFDSPTVAYSVLVVAKVEKGKSKKLKSVKELLGITIMERSSFEKNPIDFLEAKGYKEVKKDLIIKLPKYSLFELENGRKRMLASAGELQKG |
| | | NELALPSKYVNFLYLASHYEKLKGSPEDNEQKQLFVEQHKHYLDEIIEQISEFSKRVILADANLDKVLSAYNKHRDKPIREQAENIIHLFT |
| | | LTNLGAPAAFKYFDTTIDKKRYTSTKEVLDATLIHQSITGLYETRIDLSQLGGDGGGSPKKKRKVSESATPESVSGWRLFKKIS* |
| 11 | Cas9- | GGGAAGCUCAGAAUAAACGCUCAACUUUGGCCGGAUCUGCCACCAUGGCCGCCUUCAAGCCCAACCCCAUCAACUACAUCCUGGGCCUGGA |
| | Cas9 | CAUCGGCAUCGCCUCCGUGGGCUGGGCCAUGGUGGAGAUCGACGAGGAGGAGAACCCCAUCCGGCUGAUCGACCUGGGCGUGCGGGUGUUC |
| | fusion | GAGCGGGCCGAGGUGCCCAAGACCGGCGACUCCCUGGCCAUGGCCCGGCGGCUGGCCCGGUCCGUGCGGCGGCUGACCCGGCGGCGGGCCC |
| | (mRNA C)- | ACCGGCUGCUGCGGGCCCGGCGGCUGCUGAAGCGGGAGGGCGUGCUGCAGGCCGCCGACUUCGACGAGAACGGCCUGAUCAAGUCCCUGCC |
| | mRNA | CAACACCCCCUGGCAGCUGCGGGCCGCCGCCCUGGACCGGAAGCUGACCCCCCUGGAGUGGUCCGCCGUGCUGCUGCACCUGAUCAAGCAC |
| | sequence | CGGGGCUACCUGUCCCAGCGGAAGAACGAGGGCGAGACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGAGAAGGAGUCCGGCCACAUCCGGAACCAGCGGGGCGA |
| | | CUACUCCCACACCUUCUCCCGGAAGGACCUGCAGGCCGAGCUGAUCCUGCUGUUCGAGAAGCAGAAGGAGUUCGGCAACCCCCACGUGUCC |
| | | GGCGGCCUGAAGGAGGGCAUCGAGACCCUGCUGAUGACCCAGCGGCCCGCCCUGUCCGGCGACGCCGUGCAGAAGAUGCUGGGCCACUGCA |
| | | CCUUCGAGCCCGCCGAGCCCAAGGCCGCCAAGAACACCUACACCGCCGAGCGGUUCAUCUGGCUGACCAAGCUGAACAACCUGCGGAUCCU |
| | | GGAGCAGGGCUCCGAGCGGCCCCUGACCGACACCGAGCGGGCCACCCUGAUGGACGAGCCCUACCGGAAGUCCAAGCUGACCUACGCCCAG |
| | | GCCCGGAAGCUGCUGGGCCUGGAGGACACCGCCUUCUUCAAGGGCCUGCGGUACGGCAAGGACAACGCCGAGGCCUCCACCCUGAUGGAGA |
| | | UGAAGGCCUACCACGCCAUCUCCCGGGCCCUGGAGAAGGAGGGCCUGAAGGACAAGAAGUCCCCCCUGAACCUGUCCUCCGAGCUGCAGGA |
| | | CGAGAUCGGCACCGCCUUCUCCCUGUUCAAGACCGACGAGGACAUCACCGGCCGGCUGAAGGACCGGGUGCAGCCCGAGAUCCUGGAGGCC |
| | | CUGCUGAAGCACAUCUCCUUCGACAAGUUCGUGCAGAUCUCCCUGAAGGCCCUGCGGCGGAUCGUGCCCCUGAUGGAGCAGGGCAAGCGGU |
| | | ACGACGAGGCCUGCGCCGAGAUCUACGGCGACCACUACGGCAAGAAGAACACCGAGGAGAAGAUCUACCUGCCCCCCAUCCCCGCCGACGA |
| | | GAUCCGGAACCCCGUGGUGCUGCGGGCCCUGUCCCAGGCCCGGAAGGUGAUCAACGGCGUGGUGCGGCGGUACGGCUCCCCCGCCCGGAUC |
| | | CACAUCGAGACCGCCCGGGAGGUGGGCAAGUCCUUCAAGGACCGGAAGGAGAUCGAGAAGCGGCAGGAGGAGAACCGGAAGGACCGGGAGA |
| | | AGGCCGCCGCCAAGUUCCGGGAGUACUUCCCCAACUUCGUGGGCGAGCCCAAGUCCAAGGACAUCCUGAAGCUGCGGCUGUACGAGCAGCA |
| | | GCACGGCAAGUGCCUGUACUCCGGCAAGGAGAUCAACCUGGUGCGGCUGAACGAGAAGGGCUACGUGGAGAUCGACCACGCCCUGCCCUUC |
| | | UCCCGGACCUGGGACGACUCCUUCAACAACAAGGUGCUGGUGCUGGGCUCCGAGAACCAGAACAAGGGCAACCAGACCCCCUACGAGUACU |
| | | UCAACGGCAAGGACAACUCCCGGGAGUGGCAGGAGUUCAAGGCCCGGGUGGAGACCUCCCGGUUCCCCCGGUCCAAGAAGCAGCGGAUCCU |
| | | GCUGCAGAAGUUCGACGAGGACGGCUUCAAGGAGUGCAACCUGAACGACACCCGGUACGUGAACCGGUUCCUGUGCCAGUUCGUGGCCGAC |
| | | CACAUCCUGCUGACCGGCAAGGGCAAGCGGCGGGUGUUCGCCUCCAACGGCCAGAUCACCAACCUGCUGCGGGGCUUCUGGGGCCUGCGGA |
| | | AGGUGCGGGCCGAGAACGACCGGCACCACGCCCUGGACGCCGUGGUGGUGGCCUGCUCCACCGUGGCCAUGCAGCAGAAGAUCACCCGGUU |
| | | CGUGCGGUACAAGGAGAUGAACGCCUUCGACGGCAAGACCAUCGACAAGGAGACCGGCAAGGUGCUGCACCAGAAGACCCACUUCCCCCAG |
| | | CCCUGGGAGUUCUUCGCCCAGGAGGUGAUGAUCCGGGUGUUCGGCAAGCCCGACGGCAAGCCCGAGUUCGAGGAGGCCGACACCCCCGAGA |
| | | AGCUGCGGACCCUGCUGGCCGAGAAGCUGUCCUCCCGGCCCGAGGCCGUGCACGAGUACGUGACCCCCCUGUUCGUGUCCCGGGCCCCCAA |
| | | CCGGAAGAUGUCCGGCGCCCACAAGGACACCCUGCGGUCCGCCAAGCGGUUCGUGAAGCACAACGAGAAGAUCUCCGUGAAGCGGGUGUGG |
| | | CUGACCGAGAUCAAGCUGGCCGACCUGGAGAACAUGGUGAACUACAAGAACGGCCGGGAGAUCGAGCUGUACGAGGCCCUGAAGGCCCGGC |
| | | UGGAGGCCUACGGCGGCAACGCCAAGCAGGCCUUCGACCCCAAGGACAACCCCUUCUACAAGAAGGGCGGCCAGCUGGUGAAGGCCGUGCG |
| | | GGUGGAGAAGACCCAGGAGUCCGGCGUGCUGCUGAACAAGAAGAACGCCUACACCAUCGCCGACAACGGCGACAUGGUGCGGGUGGACGUG |
| | | UUCUGCAAGGUGGACAAGAAGGGCAAGAACCAGUACUUCAUCGUGCCCAUCUACGCCUGGCAGGUGGCCGAGAACAUCCUGCCCGACAUCG |
| | | ACUGCAAGGGCUACCGGAUCGACGACUCCUACACCUUCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUACCUGGCCUGGCACGACAAGGGCUCCAAGGAGCAGCAG |
| | | UUCCGGAUCUCCACCCAGAACCUGGUGCUGAUCCAGAAGUACCAGGUGAACGAGCUGGGCAAGGAGAUCCGGCCCUGCCGGCUGAAGAAGC |
| | | GGCCCCCCGUGCGGGGCGCCCCCGAGUCCGCCACCGAGUCCGGCGGCACCUCCACCGAGUCCGAGGGCUCCGCCGGCACCUCCACCGAGUC |
| | | CGAGGGCUCCGCCGGCUCCGCCGGCUCCACCUCCGGCUCCUCCUCCGGCGACAAGAAGUACUCCAUCGGCCUGGACAUCGGCACCAACUCC |
| | | GUGGGCUGGGCCGUGAUCACCGACGAGUACAAGGUGCCCUCCAAGAAGUUCAAGGUGCUGGGCAACACCGACCGGCACUCCAUCAAGAAGA |
| | | ACCUGAUCGGCGCCCUGCUGUUCGACUCCGGCGAGACCGCCGAGGCCACCCGGCUGAAGCGGACCGCCCGGCGGCGGUACACCCGGCGGAA |
| | | GAACCGGAUCUGCUACCUGCAGGAGAUCUUCUCCAACGAGAUGGCCAAGGUGGACGACUCCUUCUUCCACCGGCUGGAGGAGUCCUUCCUG |
| | | GUGGAGGAGGACAAGAAGCACGAGCGGCACCCCAUCUUCGGCAACAUCGUGGACGAGGUGGCCUACCACGAGAAGUACCCCACCAUCUACC |
| | | ACCUGCGGAAGAAGCUGGUGGACUCCACCGACAAGGCCGACCUGCGGCUGAUCUACCUGGCCCUGGCCCACAUGAUCAAGUUCCGGGGCCA |
| | | CUUCCUGAUCGAGGGCGACCUGAACCCCGACAACUCCGACGUGGACAAGCUGUUCAUCCAGCUGGUGCAGACCUACAACCAGCUGUUCGAG |
| | | GAGAACCCCAUCAACGCCUCCGGCGUGGACGCCAAGGCCAUCCUGUCCGCCCGGCUGUCCAAGUCCCGGCGGCUGGAGAACCUGAUCGCCC |
| | | AGCUGCCCGGCGAGAAGAAGAACGGCCUGUUCGGCAACCUGAUCGCCCUGUCCCUGGGCCUGACCCCCAACUUCAAGUCCAACUUCGACCU |
| | | GGCCGAGGACGCCAAGCUGCAGCUGUCCAAGGACACCUACGACGACGACCUGGACAACCUGCUGGCCCAGAUCGGCGACCAGUACGCCGAC |
| | | CUGUUCCUGGCCGCCAAGAACCUGUCCGACGCCAUCCUGCUGUCCGACAUCCUGCGGGUGAACACCGAGAUCACCAAGGCCCCCCUGUCCG |
| | | CCUCCAUGAUCAAGCGGUACGACGAGCACCACCAGGACCUGACCCUGCUGAAGGCCCUGGUGCGGCAGCAGCUGCCCGAGAAGUACAAGGA |
| | | GAUCUUCUUCGACCAGUCCAAGAACGGCUACGCCGGCUACAUCGACGGCGGCGCCUCCCAGGAGGAGUUCUACAAGUUCAUCAAGCCCAUC |
| | | CUGGAGAAGAUGGACGGCACCGAGGAGCUGCUGGUGAAGCUGAACCGGGAGGACCUGCUGCGGAAGCAGCGGACCUUCGACAACGGCUCCA |
| | | UCCCCCACCAGAUCCACCUGGGCGAGCUGCACGCCAUCCUGCGGCGGCAGGAGGACUUCUACCCCUUCCUGAAGGACAACCGGGAGAAGAU |
| | | CGAGAAGAUCCUGACCUUCCGGAUCCCCUACUACGUGGGCCCCCUGGCCCGGGGCAACUCCCGGUUCGCCUGGAUGACCCGGAAGUCCGAG |
| | | GAGACCAUCACCCCCUGGAACUUCGAGGAGGUGGUGGACAAGGGCGCCUCCGCCCAGUCCUUCAUCGAGCGGAUGACCAACUUCGACAAGA |
| | | ACCUGCCCAACGAGAAGGUGCUGCCCAAGCACUCCCUGCUGUACGAGUACUUCACCGUGUACAACGAGCUGACCAAGGUGAAGUACGUGAC |
| | | CGAGGGCAUGCGGAAGCCCGCCUUCCUGUCCGGCGAGCAGAAGAAGGCCAUCGUGGACCUGCUGUUCAAGACCAACCGGAAGGUGACCGUG |
| | | AAGCAGCUGAAGGAGGACUACUUCAAGAAGAUCGAGUGCUUCGACUCCGUGGAGAUCUCCGGCGUGGAGGACCGGUUCAACGCCUCCCUGG |
| | | GCACCUACCACGACCUGCUGAAGAUCAUCAAGGACAAGGACUUCCUGGACAACGAGGAGAACGAGGACAUCCUGGAGGACAUCGUGCUGAC |
| | | CCUGACCCUGUUCGAGGACCGGGAGAUGAUCGAGGAGCGGCUGAAGACCUACGCCCACCUGUUCGACGACAAGGUGAUGAAGCAGCUGAAG |
| | | CGGCGGCGGUACACCGGCUGGGGCCGGCUGUCCCGGAAGCUGAUCAACGGCAUCCGGGACAAGCAGUCCGGCAAGACCAUCCUGGACUUCC |
| | | UGAAGUCCGACGGCUUCGCCAACCGGAACUUCAUGCAGCUGAUCCACGACGACUCCCUGACCUUCAAGGAGGACAUCCAGAAGGCCCAGGU |
| | | GUCCGGCCAGGGCGACUCCCUGCACGAGCACAUCGCCAACCUGGCCGGCUCCCCCGCCAUCAAGAAGGGCAUCCUGCAGACCGUGAAGGUG |
| | | GUGGACGAGCUGGUGAAGGUGAUGGGCCGGCACAAGCCCGAGAACAUCGUGAUCGAGAUGGCCCGGGAGAACCAGACCACCCAGAAGGGCC |
| | | AGAAGAACUCCCGGGAGCGGAUGAAGCGGAUCGAGGAGGGCAUCAAGGAGCUGGGCUCCCAGAUCCUGAAGGAGCACCCCGUGGAGAACAC |
| | | CCAGCUGCAGAACGAGAAGCUGUACCUGUACUACCUGCAGAACGGCCGGGACAUGUACGUGGACCAGGAGCUGGACAUCAACCGGCUGUCC |
| | | GACUACGACGUGGACCACAUCGUGCCCCAGUCCUUCCUGAAGGACGACUCCAUCGACAACAAGGUGCUGACCCGGUCCGACAAGAACCGGG |
| | | GCAAGUCCGACAACGUGCCCUCCGAGGAGGUGGUGAAGAAGAUGAAGAACUACUGGCGGCAGCUGCUGAACGCCAAGCUGAUCACCCAGCG |
| | | GAAGUUCGACAACCUGACCAAGGCCGAGCGGGGCGGCCUGUCCGAGCUGGACAAGGCCGGCUUCAUCAAGCGGCAGCUGGUGGAGACCCGG |
| | | CAGAUCACCAAGCACGUGGCCCAGAUCCUGGACUCCCGGAUGAACACCAAGUACGACGAGAACGACAAGCUGAUCCGGGAGGUGAAGGUGA |
| | | UCACCCUGAAGUCCAAGCUGGUGUCCGACUUCCGGAAGGACUUCCAGUUCUACAAGGUGCGGGAGAUCAACAACUACCACCACGCCCACGA |
| | | CGCCUACCUGAACGCCGUGGUGGGCACCGCCCUGAUCAAGAAGUACCCCAAGCUGGAGUCCGAGUUCGUGUACGGCGACUACAAGGUGUAC |
| | | GACGUGCGGAAGAUGAUCGCCAAGUCCGAGCAGGAGAUCGGCAAGGCCACCGCCAAGUACUUCUUCUACUCCAACAUCAUGAACUUCUUCA |
| | | AGACCGAGAUCACCCUGGCCAACGGCGAGAUCCGGAAGCGGCCCCUGAUCGAGACCAACGGCGAGACCGGCGAGAUCGUGUGGGACAAGGG |
| | | CCGGGACUUCGCCACCGUGCGGAAGGUGCUGUCCAUGCCCCAGGUGAACAUCGUGAAGAAGACCGAGGUGCAGACCGGCGGCUUCUCCAAG |
| | | GAGUCCAUCCUGCCCAAGCGGAACUCCGACAAGCUGAUCGCCCGGAAGAAGGACUGGGACCCCAAGAAGUACGGCGGCUUCGACUCCCCCA |
| | | CCGUGGCCUACUCCGUGCUGGUGGUGGCCAAGGUGGAGAAGGGCAAGUCCAAGAAGCUGAAGUCCGUGAAGGAGCUGCUGGGCAUCACCAU |
| | | CAUGGAGCGGUCCUCCUUCGAGAAGAACCCCAUCGACUUCCUGGAGGCCAAGGGCUACAAGGAGGUGAAGAAGGACCUGAUCAUCAAGCUG |
| | | CCCAAGUACUCCCUGUUCGAGCUGGAGAACGGCCGGAAGCGGAUGCUGGCCUCCGCCGGCGAGCUGCAGAAGGGCAACGAGCUGGCCCUGC |
| | | CCUCCAAGUACGUGAACUUCCUGUACCUGGCCUCCCACUACGAGAAGCUGAAGGGCUCCCCCGAGGACAACGAGCAGAAGCAGCUGUUCGU |
| | | GGAGCAGCACAAGCACUACCUGGACGAGAUCAUCGAGCAGAUCUCCGAGUUCUCCAAGCGGGUGAUCCUGGCCGACGCCAACCUGGACAAG |
| | | GUGCUGUCCGCCUACAACAAGCACCGGGACAAGCCCAUCCGGGAGCAGGCCGAGAACAUCAUCCACCUGUUCACCCUGACCAACCUGGGCG |
| | | CCCCCGCCGCCUUCAAGUACUUCGACACCACCAUCGACAAGAAGCGGUACACCUCCACCAAGGAGGUGCUGGACGCCACCCUGAUCCACCA |
| | | GUCCAUCACCGGCCUGUACGAGACCCGGAUCGACCUGUCCCAGCUGGGGGGCGACGGCGGCGGCUCCCCCAAGAAGAAGCGGAAGGUGUCC |
| | | GAGUCCGCCACCCCCGAGUCCGUGUCCGGCUGGCGGCUGUUCAAGAAGAUCUCCUAGCUAGCACCAGCCUCAAGAACACCCGAAUGGAGUC |
| | | UCUAAGCUACAUAAUACCAACUUACACUUUACAAAAUGUUGUCCCCCAAAAUGUAGCCAUUCGUAUCUGCUCCUAAUAAAAAGAAAGUUUC |
| | | UUCACAUUCUCUCGAGAAAAAAAAAAAAUGGAAAAAAAAAAAACGGAAAAAAAAAAAAGGUAAAAAAAAAAAAUAUAAAAAAAAAAAACAU |
| | | AAAAAAAAAAAACGAAAAAAAAAAAACGUAAAAAAAAAAAACUCAAAAAAAAAAAGAUAAAAAAAAAAAACCUAAAAAAAAAAAAUGUAAA |
| | | AAAAAAAAAGGGAAAAAAAAAAAACGCAAAAAAAAAAAACACAAAAAAAAAAAAUGCAAAAAAAAAAAAUCGAAAAAAAAAAAAUCUAAAA |
| | | AAAAAAAACGAAAAAAAAAAAACCCAAAAAAAAAAAAGACAAAAAAAAAAAAUAGAAAAAAAAAAAGUUAAAAAAAAAAAACUGAAAAAAA |
| | | AAAAAUUUAAAAAAAAAAAAUCUAG |
| 12 | Cas9- | auggccgccuucaagcccaaccccaucaacuacauccugggccuggacaucggcaucgccuccgugggcugggccaugguggagaucgacg |
| | Cas9 | aggaggagaaccccauccggcugaucgaccugggcgugcggguguucgagcgggccgaggugcccaagaccggcgacucccuggccauggc |
| | fusion | ccggcggcuggcccgguccgugcggcggcugacccggcggcgggcccaccggcugcugcgggcccggcggcugcugaaggggagcggcgug |
| | (mRNA C)- | cugcaggccgccgacuucgacgagaacggccugaucaagucccugcccaacacccccuggcagcugcgggccgccgcccuggaccggaagc |
| | ORF | ugaccccccuggagugguccgccgugcugcugcaccugaucaagcaccggggcuaccugucccagcggaagaacgagggcgagaccgccga |
| | sequence | caaggagcugggcgcccugcugaagggcguggccaacaacgcccacgcccugcagaccggcgacuuccggacccccgccgagcuggcccug |
| | | aacaaguucgagaaggaguccggccacauccggaaccagcggcgggacuacucccacaccuucucccggaaggaccugcaggccgagcuga |
| | | uccugcuguucgagaagcagaaggaguucggcaacccccacguguccggcggccugaaggagggcaucgagacccugcugaugacccagcg |
| | | gcccgcccuguccggcgacgccgugcagaagaugcugggccacugcaccuucgagcccgccgagcccaaggccgccaagaacaccuacacc |
| | | gccgagcgguucaucuggcugaccaagcugaacaaccugcggauccuggagcagggcuccgagcggccccugaccgacaccgagcgggcca |
| | | cccugauggacgagcccuaccggaaguccaagcugaccuacgcccaggcccggaagcugcugggccuggaggacaccgccuucuucaaggg |
| | | ccugcgguacggcaaggacaacgccgaggccuccacccugauggagaugaaggccuaccacgccaucucccgggcccuggagaaggagggc |
| | | cugaaggacaagaaguccccccugaaccuguccuccgagcugcaggacgagaucggcaccgccuucucccuguucaagaccgacgaggaca |
| | | ucaccggccggcugaaggaccgggugcagcccgagauccuggaggcccugcugaagcacaucuccuucgacaaguucgugcagaucucccu |
| | | gaaggcccugcggcggaucgugccccugauggagcagggcaagcgguacgacgaggccugcgccgagaucuacggcgaccacuacggcaag |
| | | aagaacaccgaggagaagaucuaccugccccccauccccgccgacgagauccggaaccccguggugcugcgggcccugucccaggcccgga |
| | | aggugaucaacggcguggugcggcgguacggcucccccgcccggauccacaucgagaccgcccgggaggugggcaaguccuucaaggaccg |
| | | gaaggagaucgagaagcggcaggaggagaaccggaaggaccgggagaaggccgccgccaaguuccgggaguacuuccccaacuucgugggc |
| | | gagcccaaguccaaggacauccugaagcugcggcuguacgagcagcagcacggcaagugccuguacuccggcaaggagaucaaccuggugc |
| | | ggcugaacgagaagggcuacguggagaucgaccacgcccugcccuucucccggaccugggacgacuccuucaacaacaaggugcuggugcu |
| | | gggcuccgagaaccagaacaagggcaaccagacccccuacgaguacuucaacggcaaggacaacucccgggaguggcaggaguucaaggcc |
| | | cggguggagaccucccgguucccccgguccaagaagcagcggauccugcugcagaaguucgacgaggacggcuucaaggagugcaaccuga |
| | | acgacacccgguacgugaaccgguuccugugccaguucguggccgaccacauccugcugaccggcaagggcaagggcggcguguucgccuc |
| | | caacggccagaucaccaaccugcugcggggcuucuggggccugcggaagguggggcccgagaacgaccggcaccacgcccuggacgccgug |
| | | gugguggccugcuccaccguggccaugcagcagaagaucacccgguucgugcgguacaaggagaugaacgccuucgacggcaagaccaucg |
| | | acaaggagaccggcaaggugcugcaccagaagacccacuucccccagcccuggcgaguuuucgcccaggaggugaugauccggguguucgg |
| | | caagcccgacggcaagcccgaguucgaggaggccgacacccccgagaagcugcggacccugcuggccgagaagcuguccucccggcccgag |
| | | gccgugcacgaguacgugaccccccuguucgugucccgggcccccaaccggaagauguccggcgcccacaaggacacccugcgguccgcca |
| | | agcgguucgugaagcacaacgagaagaucuccgugaagcggguguggcugaccgagaucaagcuggccgaccuggagaacauggugaacua |
| | | caagaacggccgggagaucgagcuguacgaggcccugaaggcccggcuggaggccuacggcggcaacgccaagcaggccuucgaccccaag |
| | | gacaaccccuucuacaagaagggcggccagcuggugaaggccgugcggguggagaagacccaggaguccggcgugcugcugaacaagaaga |
| | | acgccuacaccaucgccgacaacggcgacauggugcggguggacguguucugcaagguggacaagaagggcaagaaccaguacuucaucgu |
| | | gcccaucuacgccuggcagguggccgagaacauccugcccgacaucgacugcaagggcuaccggaucgacgacuccuacaccuucugcuuc |
| | | ucccugcacaaguacgaccugaucgccuuccagaaggacgagaaguccaagguggaguucgccuacuacaucaacugcgacuccuccaacg |
| | | gccgguucuaccuggccuggcacgacaagggcuccaaggagcagcaguuccggaucuccacccagaaccuggugcugauccagaaguacca |
| | | ggugaacgagcugggcaaggagauccggcccugccggcugaagaagcggccccccgugcggGGCGCCCCCGAGUCCGCCACCGAGUCCGGC |
| | | GGCACCUCCACCGAGUCCGAGGGCUCCGCCGGCACCUCCACCGAGUCCGAGGGCUCCGCCGGCUCCGCCGGCUCCACCUCCGGCUCCUCCU |
| | | CCGGCGACAAGAAGUACUCCAUCGGCCUGGACAUCGGCACCAACUCCGUGGGCUGGGCCGUGAUCACCGACGAGUACAAGGUGCCCUCCAA |
| | | GAAGUUCAAGGUGCUGGGCAACACCGACCGGCACUCCAUCAAGAAGAACCUGAUCGGCGCCCUGCUGUUCGACUCCGGCGAGACCGCCGAG |
| | | GCCACCCGGCUGAAGCGGACCGCCCGGCGGCGGUACACCCGGCGGAAGAACCGGAUCUGCUACCUGCAGGAGAUCUUCUCCAACGAGAUGG |
| | | CCAAGGUGGACGACUCCUUCUUCCACCGGCUGGAGGAGUCCUUCCUGGUGGAGGAGGACAAGAAGCACGAGCGGCACCCCAUCUUCGGCAA |
| | | CAUCGUGGACGAGGUGGCCUACCACGAGAAGUACCCCACCAUCUACCACCUGCGGAAGAAGCUGGUGGACUCCACCGACAAGGCCGACCUG |
| | | CGGCUGAUCUACCUGGCCCUGGCCCACAUGAUCAAGUUCCGGGGCCACUUCCUGAUCGAGGGCGACCUGAACCCCGACAACUCCGACGUGG |
| | | ACAAGCUGUUCAUCCAGCUGGUGCAGACCUACAACCAGCUGUUCGAGGAGAACCCCAUCAACGCCUCCGGCGUGGACGCCAAGGCCAUCCU |
| | | GUCCGCCCGGCUGUCCAAGUCCCGGCGGCUGGAGAACCUGAUCGCCCAGCUGCCCGGCGAGAAGAAGAACGGCCUGUUCGGCAACCUGAUC |
| | | GCCCUGUCCCUGGGCCUGACCCCCAACUUCAAGUCCAACUUCGACCUGGCCGAGGACGCCAAGCUGCAGCUGUCCAAGGACACCUACGACG |
| | | ACGACCUGGACAACCUGCUGGCCCAGAUCGGCGACCAGUACGCCGACCUGUUCCUGGCCGCCAAGAACCUGUCCGACGCCAUCCUGCUGUC |
| | | CGACAUCCUGCGGGUGAACACCGAGAUCACCAAGGCCCCCCUGUCCGCCUCCAUGAUCAAGCGGUACGACGAGCACCACCAGGACCUGACC |
| | | CUGCUGAAGGCCCUGGUGCGGCAGCAGCUGCCCGAGAAGUACAAGGAGAUCUUCUUCGACCAGUCCAAGAACGGCUACGCCGGCUACAUCG |
| | | ACGGCGGCGCCUCCCAGGAGGAGUUCUACAAGUUCAUCAAGCCCAUCCUGGAGAAGAUGGACGGCACCGAGGAGCUGCUGGUGAAGCUGAA |
| | | CCGGGAGGACCUGCUGCGGAAGCAGCGGACCUUCGACAACGGCUCCAUCCCCCACCAGAUCCACCUGGGCGAGCUGCACGCCAUCCUGCGG |
| | | CGGCAGGAGGACUUCUACCCCUUCCUGAAGGACAACCGGGAGAAGAUCGAGAAGAUCCUGACCUUCCGGAUCCCCUACUACGUGGGCCCCC |
| | | UGGCCCGGGGCAACUCCCGGUUCGCCUGGAUGACCCGGAAGUCCGAGGAGACCAUCACCCCCUGGAACUUCGAGGAGGUGGUGGACAAGGG |
| | | CGCCUCCGCCCAGUCCUUCAUCGAGCGGAUGACCAACUUCGACAAGAACCUGCCCAACGAGAAGGUGCUGCCCAAGCACUCCCUGCUGUAC |
| | | GAGUACUUCACCGUGUACAACGAGCUGACCAAGgugaaguacgugaccgagggcaugcggaagcccgccuuccuguccggcgagcagaaga |
| | | aggccaucguggaccugcuguucaagaccaaccggaaggugaccgugaagcagcugaaggaggacuacuucaagaagaucgagugcuucga |
| | | cuccguggagaucuccggcguggaggaccgguucaacgccucccugggcaccuaccacgaccugcugaagaucaucaaggacaaggacuuc |
| | | cuggacaacgaggagaacgaggacauccuggaggacaucgugcugacccugacccuguucgaggaccgggagaugaucgaggagcggcuga |
| | | agaccuacgcccaccuguucgacgacaaggugaugaagcagcugaagcggcggcgguacaccggcuggggccggcugucccggaagcugau |
| | | caacggcauccgggacaagcaguccggcaagaccauccuggacuuccugaaguccgacggcuucgccaaccggaacuucaugcagcugauc |
| | | cacgacgacucccugaccuucaaggaggacauccagaaggcccagguguccggccagggcgacucccugcacgagcacaucgccaaccugg |
| | | ccggcucccccgccaucaagaagggcauccugcagaccgugaaggugguggacgagcuggugaaggugaugggccggcacaagcccgagaa |
| | | caucgugaucgagauggcccgggagaaccagaccacccagaagggccagaagaacucccgggagcggaugaagcggaucgaggagggcauc |
| | | aaggagcugggcucccagauccugaaggagcaccccguggagaacacccagcugcagaacgagaagcuguaccuguacuaccugcagaacg |
| | | gccgggacauguacguggaccaggagcuggacaucaaccggcuguccgacuacgacguggaccacaucgugccccaguccuuccugaagga |
| | | cgacuccaucgacaacaaggugcugacccgguccgacaagaaccggggcaaguccgacaacgugcccuccgaggagguggugaagaagaug |
| | | aagaacuacuggcggcagcugcugaacgccaagcugaucacccagcggaaguucgacaaccugaccaaggccgagcggggggccuguccg |
| | | agcuggacaaggccggcuucaucaagcggcagcugguggagacccggcagaucaccaagcacguggcccagauccuggacucccggaugaa |
| | | caccaaguacgacgagaacgacaagcugauccgggaggugaaggugaucacccugaaguccaagcugguguccgacuuccggaaggacuuc |
| | | caguucuacaaggugcgggagaucaacaacuaccaccacgcccacgacgccuaccugaacgccguggugggcaccgcccugaucaagaagu |
| | | accccaagcuggaguccgaguucguguacggcgacuacaagguguacgacgugcggaagaugaucgccaaguccgagcaggagaucggcaa |
| | | ggccaccgccaaguacuucuucuacuccaacaucaugaacuucuucaagaccgagaucacccuggccaacggcgagauccggaagcggccc |
| | | cugaucgagaccaacggcgagaccggcgagaucgugugggacaagggccgggacuucgccaccgugcggaaggugcuguccaugccccagg |
| | | ugaacaucgugaagaagaccgaggugcagaccggcggcuucuccaaggaguccauccugcccaagcggaacuccgacaagcugaucgcccg |
| | | gaagaaggacugggaccccaagaaguacggcggcuucgacucccccaccguggccuacuccgugcuggugguggccaagguggagaagggc |
| | | aaguccaagaagcugaaguccgugaaggagcugcugggcaucaccaucauggagcgguccuccuucgagaagaaccccaucgacuuccugg |
| | | aggccaagggcuacaaggaggugaagaaggaccugaucaucaagcugcccaaguacucccuguucgagcuggagaacggccggaagcggau |
| | | gcuggccuccgccggcgagcugcagaagggcaacgagcuggcccugcccuccaaguacgugaacuuccuguaccuggccucccacuacgag |
| | | aagcugaagggcucccccgaggacaacgagcagaagcagcuguucguggagcagcacaagcacuaccuggacgagaucaucgagcagaucu |
| | | ccgaguucuccaagcgggugauccuggccgacgccaaccuggacaaggugcuguccgccuacaacaagcaccgggacaagcccauccggga |
| | | gcaggccgagaacaucauccaccuguucacccugaccaaccugggcgcccccgccgccuucaaguacuucgacaccaccaucgacAAGaag |
| | | cgguacaccuccaccaaggaggugcuggacgccacccugauccaccaguccaucaccggccuguacgagacccggaucgaccugucccagc |
| | | ugggcggcgacGGCGGCGGCUCCCCCAAGAAGAAGCGGAAGGUGUCCGAGUCCGCCACCCCCGAGUCCGUGUCCGGCUGGCGGCUGUUCAA |
| | | GAAGAUCUCCuag |
| 13 | Cas9- | MAAFKPNPINYILGLDIGIASVGWAMVEIDEEENPIRLIDLGVRVFERAEVPKTGDSLAMARRLARSVRRLTRRRAHRLLRARRLLKREGV |
| | Cas9 | LQAADFDENGLIKSLPNTPWQLRAAALDRKLTPLEWSAVLLHLIKHRGYLSQRKNEGETADKELGALLKGVANNAHALQTGDFRTPAELAL |
| | fusion | NKFEKESGHIRNQRGDYSHTFSRKDLQAELILLFEKQKEFGNPHVSGGLKEGIETLLMTQRPALSGDAVQKMLGHCTFEPAEPKAAKNTYT |
| | (mRNA C)- | AERFIWLTKLNNLRILEQGSERPLTDTERATLMDEPYRKSKLTYAQARKLLGLEDTAFFKGLRYGKDNAEASTLMEMKAYHAISRALEKEG |
| | amino | LKDKKSPLNLSSELQDEIGTAFSLFKTDEDITGRLKDRVQPEILEALLKHISFDKFVQISLKALRRIVPLMEQGKRYDEACAEIYGDHYGK |
| | acid | KNTEEKIYLPPIPADEIRNPVVLRALSQARKVINGVVRRYGSPARIHIETAREVGKSFKDRKEIEKRQEENRKDREKAAAKFREYFPNFVG |
| | sequence | EPKSKDILKLRLYEQQHGKCLYSGKEINLVRLNEKGYVEIDHALPFSRTWDDSFNNKVLVLGSENQNKGNQTPYEYFNGKDNSREWQEFKA |
| | | RVETSRFPRSKKQRILLQKFDEDGFKECNLNDTRYVNRFLCQFVADHILLTGKGKRRVFASNGQITNLLRGFWGLRKVRAENDRHHALDAV |
| | | VVACSTVAMQQKITRFVRYKEMNAFDGKTIDKETGKVLHQKTHFPQPWEFFAQEVMIRVFGKPDGKPEFEEADTPEKLRTLLAEKLSSRPE |
| | | AVHEYVTPLFVSRAPNRKMSGAHKDTLRSAKRFVKHNEKISVKRVWLTEIKLADLENMVNYKNGREIELYEALKARLEAYGGNAKQAFDPK |
| | | DNPFYKKGGQLVKAVRVEKTQESGVLLNKKNAYTIADNGDMVRVDVFCKVDKKGKNQYFIVPIYAWQVAENILPDIDCKGYRIDDSYTFCF |
| | | SLHKYDLIAFQKDEKSKVEFAYYINCDSSNGRFYLAWHDKGSKEQQFRISTQNLVLIQKYQVNELGKEIRPCRLKKRPPVRGAPESATESG |
| | | GTSTESEGSAGTSTESEGSAGSAGSTSGSSSGDKKYSIGLDIGTNSVGWAVITDEYKVPSKKFKVLGNTDRHSIKKNLIGALLFDSGETAE |
| | | ATRLKRTARRRYTRRKNRICYLQEIFSNEMAKVDDSFFHRLEESFLVEEDKKHERHPIFGNIVDEVAYHEKYPTIYHLRKKLVDSTDKADL |
| | | RLIYLALAHMIKFRGHFLIEGDLNPDNSDVDKLFIQLVQTYNQLFEENPINASGVDAKAILSARLSKSRRLENLIAQLPGEKKNGLFGNLI |
| | | ALSLGLTPNFKSNFDLAEDAKLQLSKDTYDDDLDNLLAQIGDQYADLFLAAKNLSDAILLSDILRVNTEITKAPLSASMIKRYDEHHQDLT |
| | | LLKALVRQQLPEKYKEIFFDQSKNGYAGYIDGGASQEEFYKFIKPILEKMDGTEELLVKLNREDLLRKQRTFDNGSIPHQIHLGELHAILR |
| | | RQEDFYPFLKDNREKIEKILTFRIPYYVGPLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEISGVEDRFNASLGTYHDLLKIIKDKDF |
| | | LDNEENEDILEDIVLTLTLFEDREMIEERLKTYAHLFDDKVMKQLKRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKVVDELVKVMGRHKPENIVIEMARENQTTQKGQKNSRERMKRIEEGI |
| | | KELGSQILKEHPVENTQLQNEKLYLYYLQNGRDMYVDQELDINRLSDYDVDHIVPQSFLKDDSIDNKVLTRSDKNRGKSDNVPSEEVVKKM |
| | | KNYWRQLLNAKLITQRKFDNLTKAERGGLSELDKAGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKHRDKPIREQAENIIHLFTLTNLGAPAAFKYFDTTIDKK |
| | | RYTSTKEVLDATLIHQSITGLYETRIDLSQLGGDGGGSPKKKRKVSESATPESVSGWRLFKKIS* |
| 14 | Cas9- | GGGAAGCUCAGAAUAAACGCUCAACUUUGGCCGGAUCUGCCACCAUGGACGGCUCCGGCGGCGGCUCCCCCAAGAAGAAGCGGAAGGUGGA |
| | Cas9 | GGACAAGCGGCCCGCCGCCACCAAGAAGGCCGGCCAGGCCAAGAAGAAGAAGGGCGGCUCCGGCGGCGGCGCCGCCUUCAAGCCCAACCCC |
| | fusion | AUCAACUACAUCCUGGGCCUGGACAUCGGCAUCGCCUCCGUGGGCUGGGCCAUGGUGGAGAUCGACGAGGAGGAGAACCCCAUCCGGCUGA |
| | (mRNA D)- | UCGACCUGGGCGUGCGGGUGUUCGAGCGGGCCGAGGUGCCCAAGACCGGCGACUCCCUGGCCAUGGCCCGGCGGCUGGCCCGGUCCGUGCG |
| | mRNA | GCGGCUGACCCGGCGGCGGGCCCACCGGCUGCUGCGGGCCCGGCGGCUGCUGAAGCGGGAGGGCGUGCUGCAGGCCGCCGACUUCGACGAG |
| | sequence | AACGGCCUGAUCAAGUCCCUGCCCAACACCCCCUGGCAGCUGCGGGCCGCCGCCCUGGACCGGAAGCUGACCCCCCUGGAGUGGUCCGCCG |
| | | UGCUGCUGCACCUGAUCAAGCACCGGGGCUACCUGUCCCAGCGGAAGAACGAGGGCGAGACCGCCGACAAGGAGCUGGGCGCCCUGCUGAA |
| | | GGGCGUGGCCAACAACGCCCACGCCCUGCAGACCGGCGACUUCCGGACCCCCGCCGAGCUGGCCCUGAACAAGUUCGAGAAGGAGUCCGGC |
| | | CACAUCCGGAACCAGCGGGGCGACUACUCCCACACCUUCUCCCGGAAGGACCUGCAGGCCGAGCUGAUCCUGCUGUUCGAGAAGCAGAAGG |
| | | AGUUCGGCAACCCCCACGUGUCCGGCGGCCUGAAGGAGGGCAUCGAGACCCUGCUGAUGACCCAGCGGCCCGCCCUGUCCGGCGACGCCGU |
| | | GCAGAAGAUGCUGGGCCACUGCACCUUCGAGCCCGCCGAGCCCAAGGCCGCCAAGAACACCUACACCGCCGAGCGGUUCAUCUGGCUGACC |
| | | AAGCUGAACAACCUGCGGAUCCUGGAGCAGGGCUCCGAGCGGCCCCUGACCGACACCGAGCGGGCCACCCUGAUGGACGAGCCCUACCGGA |
| | | AGUCCAAGCUGACCUACGCCCAGGCCCGGAAGCUGCUGGGCCUGGAGGACACCGCCUUCUUCAAGGGCCUGCGGUACGGCAAGGACAACGC |
| | | CGAGGCCUCCACCCUGAUGGAGAUGAAGGCCUACCACGCCAUCUCCCGGGCCCUGGAGAAGGAGGGCCUGAAGGACAAGAAGUCCCCCCUG |
| | | AACCUGUCCUCCGAGCUGCAGGACGAGAUCGGCACCGCCUUCUCCCUGUUCAAGACCGACGAGGACAUCACCGGCCGGCUGAAGGACCGGG |
| | | UGCAGCCCGAGAUCCUGGAGGCCCUGCUGAAGCACAUCUCCUUCGACAAGUUCGUGCAGAUCUCCCUGAAGGCCCUGCGGCGGAUCGUGCC |
| | | CCUGAUGGAGCAGGGCAAGCGGUACGACGAGGCCUGCGCCGAGAUCUACGGCGACCACUACGGCAAGAAGAACACCGAGGAGAAGAUCUAC |
| | | CUGCCCCCCAUCCCCGCCGACGAGAUCCGGAACCCCGUGGUGCUGCGGGCCCUGUCCCAGGCCCGGAAGGUGAUCAACGGCGUGGUGCGGC |
| | | GGUACGGCUCCCCCGCCCGGAUCCACAUCGAGACCGCCCGGGAGGUGGGCAAGUCCUUCAAGGACCGGAAGGAGAUCGAGAAGCGGCAGGA |
| | | GGAGAACCGGAAGGACCGGGAGAAGGCCGCCGCCAAGUUCCGGGAGUACUUCCCCAACUUCGUGGGCGAGCCCAAGUCCAAGGACAUCCUG |
| | | AAGCUGCGGCUGUACGAGCAGCAGCACGGCAAGUGCCUGUACUCCGGCAAGGAGAUCAACCUGGUGCGGCUGAACGAGAAGGGCUACGUGG |
| | | AGAUCGACCACGCCCUGCCCUUCUCCCGGACCUGGGACGACUCCUUCAACAACAAGGUGCUGGUGCUGGGCUCCGAGAACCAGAACAAGGG |
| | | CAACCAGACCCCCUACGAGUACUUCAACGGCAAGGACAACUCCCGGGAGUGGCAGGAGUUCAAGGCCCGGGUGGAGACCUCCCGGUUCCCC |
| | | CGGUCCAAGAAGCAGCGGAUCCUGCUGCAGAAGUUCGACGAGGACGGCUUCAAGGAGUGCAACCUGAACGACACCCGGUACGUGAACCGGU |
| | | UCCUGUGCCAGUUCGUGGCCGACCACAUCCUGCUGACCGGCAAGGGCAAGCGGCGGGUGUUCGCCUCCAACGGCCAGAUCACCAACCUGCU |
| | | GCGGGGCUUCUGGGGCCUGCGGAAGGUGCGGGCCGAGAACGACCGGCACCACGCCCUGGACGCCGUGGUGGUGGCCUGCUCCACCGUGGCC |
| | | AUGCAGCAGAAGAUCACCCGGUUCGUGCGGUACAAGGAGAUGAACGCCUUCGACGGCAAGACCAUCGACAAGGAGACCGGCAAGGUGCUGC |
| | | ACCAGAAGACCCACUUCCCCCAGCCCUGGGAGUUCUUCGCCCAGGAGGUGAUGAUCCGGGUGUUCGGCAAGCCCGACGGCAAGCCCGAGUU |
| | | CGAGGAGGCCGACACCCCCGAGAAGCUGCGGACCCUGCUGGCCGAGAAGCUGUCCUCCCGGCCCGAGGCCGUGCACGAGUACGUGACCCCC |
| | | CUGUUCGUGUCCCGGGCCCCCAACCGGAAGAUGUCCGGCGCCCACAAGGACACCCUGCGGUCCGCCAAGCGGUUCGUGAAGCACAACGAGA |
| | | AGAUCUCCGUGAAGCGGGUGUGGCUGACCGAGAUCAAGCUGGCCGACCUGGAGAACAUGGUGAACUACAAGAACGGCCGGGAGAUCGAGCU |
| | | GUACGAGGCCCUGAAGGCCCGGCUGGAGGCCUACGGCGGCAACGCCAAGCAGGCCUUCGACCCCAAGGACAACCCCUUCUACAAGAAGGGC |
| | | GGCCAGCUGGUGAAGGCCGUGCGGGUGGAGAAGACCCAGGAGUCCGGCGUGCUGCUGAACAAGAAGAACGCCUACACCAUCGCCGACAACG |
| | | GCGACAUGGUGCGGGUGGACGUGUUCUGCAAGGUGGACAAGAAGGGCAAGAACCAGUACUUCAUCGUGCCCAUCUACGCCUGGCAGGUGGC |
| | | CGAGAACAUCCUGCCCGACAUCGACUGCAAGGGCUACCGGAUCGACGACUCCUACACCUUCUGCUUCUCCCUGCACAAGUACGACCUGAUC |
| | | GCCUUCCAGAAGGACGAGAAGUCCAAGGUGGAGUUCGCCUACUACAUCAACUGCGACUCCUCCAACGGCCGGUUCUACCUGGCCUGGCACG |
| | | ACAAGGGCUCCAAGGAGCAGCAGUUCCGGAUCUCCACCCAGAACCUGGUGCUGAUCCAGAAGUACCAGGUGAACGAGCUGGGCAAGGAGAU |
| | | CCGGCCCUGCCGGCUGAAGAAGCGGCCCCCCGUGCGGUAGCUAGCACCAGCCUCAAGAACACCCGAAUGGAGUCUCUAAGCUACAUAAUAC |
| | | CAACUUACACUUUACAAAAUGUUGUCCCCCAAAAUGUAGCCAUUCGUAUCUGCUCCUAAUAAAAAGAAAGUUUCUUCACAUUCUCUCGAGA |
| | | AAAAAAAAAAAUGGAAAAAAAAAAAACGGAAAAAAAAAAAAGGUAAAAAAAAAAAAUAUAAAAAAAAAAAACAUAAAAAAAAAAAACGAAA |
| | | AAAAAAAAACGUAAAAAAAAAAAACUCAAAAAAAAAAAGAUAAAAAAAAAAAACCUAAAAAAAAAAAAUGUAAAAAAAAAAAAGGGAAAAA |
| | | AAAAAAACGCAAAAAAAAAAAACACAAAAAAAAAAAAUGCAAAAAAAAAAAAUCGAAAAAAAAAAAAUCUAAAAAAAAAAAACGAAAAAAA |
| | | AAAAACCCAAAAAAAAAAAAGACAAAAAAAAAAAAUAGAAAAAAAAAAAGUUAAAAAAAAAAAACUGAAAAAAAAAAAAUUUAAAAAAAAA |
| | | AAAUCUAG |
| 15 | Cas9- | augGACGGCUCCGGCGGCGGCUCCCCCAAGAAGAAGCGGAAGGUGGAGGACAAGCGGCCCGCCGCCACCAAGAAGGCCGGCCAGGCCAAGA |
| | Cas9 | AGAAGAAGGGCGGCUCCGGCGGCGGCgccgccuucaagcccaaccccaucaacuacauccugggccuggacaucggcaucgccuccguggg |
| | fusion | cugggccaugguggagaucgacgaggaggagaaccccauccggcugaucgaccugggcgugcggguguucgagcgggccgaggugcccaag |
| | (mRNA D)- | accggcgacucccuggccauggcccgcgggcuggcccgguccguggcgcggcugacccggcgggcggcccaccggcugcuggcggcccggc |
| | ORF | ggcugcugaagcgggagggcgugcugcaggccgccgacuucgacgagaacggccugaucaagucccugcccaacacccccuggcagcugcg |
| | sequence | ggccgccgcccuggaccggaagcugaccccccuggagugguccgccgugcugcugcaccugaucaagcaccggggcuaccugucccagcgg |
| | | aagaacgagggcgagaccgccgacaaggagcugggcgcccugcugaagggcguggccaacaacgcccacgcccugcagaccggcgacuucc |
| | | ggacccccgccgagcuggcccugaacaaguucgagaaggaguccggccacauccggaaccagcggggcgacuacucccacaccuucucccg |
| | | gaaggaccugcaggccgagcugauccugcuguucgagaagcagaaggaguucggcaacccccacguguccggcggccugaaggagggcauc |
| | | gagacccugcugaugacccagcggcccgcccuguccggcgacgccgugcagaagaugcugggccacugcaccuucgagcccgccgagccca |
| | | aggccgccaagaacaccuacaccgccgagcgguucaucuggcugaccaagcugaacaaccugcggauccuggagcagggcuccgagcggcc |
| | | ccugaccgacaccgagcgggccacccugauggacgagcccuaccggaaguccaagcugaccuacgcccaggcccggaagcugcugggccug |
| | | gaggacaccgccuucuucaagggccugcgguacggcaaggacaacgccgaggccuccacccugauggagaugaaggccuaccacgccaucu |
| | | cccgggcccuggagaaggagggccugaaggacaagaaguccccccugaaccuguccuccgagcugcaggacgagaucggcaccgccuucuc |
| | | ccuguucaagaccgacgaggacaucaccggccggcugaaggaccgggugcagcccgagauccuggaggcccugcugaagcacaucuccuuc |
| | | gacaaguucgugcagaucucccugaaggcccugcggcggaucgugccccugauggagcagggcaagcgguacgacgaggccugcgccgaga |
| | | ucuacggcgaccacuacggcaagaagaacaccgaggagaagaucuaccugccccccauccccgccgacgagauccggaaccccguggugcu |
| | | gcgggcccugucccaggcccggaaggugaucaacggcguggugcggcgguacggcucccccgcccggauccacaucgagaccgcccgggag |
| | | gugggcaaguccuucaaggaccggaaggagaucgagaagcggcaggaggagaaccggaaggaccgggagaaggccgccgccaaguuccggg |
| | | aguacuuccccaacuucgugggcgagcccaaguccaaggacauccugaagcugcggcuguacgagcagcagcacggcaagugccuguacuc |
| | | cggcaaggagaucaaccuggugcggcugaacgagaagggcuacguggagaucgaccacgcccugcccuucucccggaccugggacgacucc |
| | | uucaacaacaaggugcuggugcugggcuccgagaaccagaacaagggcaaccagacccccuacgaguacuucaacggcaaggacaacuccc |
| | | gggaguggcaggaguucaaggcccggguggagaccucccgguucccccgguccaagaagcagcggauccugcugcagaaguucgacgagga |
| | | cggcuucaaggagugcaaccugaacgacacccgguacgugaaccgguuccugugccaguucguggccgaccacauccugcugaccggcaag |
| | | ggcaagcggcggguguucgccuccaacggccagaucaccaaccugcugcggggcuucugggcgccugcggaagguggggccgagaacgacc |
| | | ggcaccacgcccuggacgccguggugguggccugcuccaccguggccaugcagcagaagaucacccgguucgugcgguacaaggagaugaa |
| | | cgccuucgacggcaagaccaucgacaaggagaccggcaaggugcugcaccagaagacccacuucccccagcccugggaguucuucgcccag |
| | | gaggugaugauccggguguucggcaagcccgacggcaagcccgaguucgaggaggccgacacccccgagaagcugcggacccugcuggccg |
| | | agaagcuguccucccggcccgaggccgugcacgaguacgugaccccccuguucgugucccgggcccccaaccggaagauguccggcgccca |
| | | caaggacacccugcgguccgccaagcgguucgugaagcacaacgagaagaucuccgugaagcggguguggcugaccgagaucaagcuggcc |
| | | gaccuggagaacauggugaacuacaagaacggccgggagaucgagcuguacgaggcccugaaggcccggcuggaggccuacggcggcaacg |
| | | ccaagcaggccuucgaccccaaggacaaccccuucuacaagaagggcggccagcuggugaaggccgugcggguggagaagacccaggaguc |
| | | cggcgugcugcugaacaagaagaacgccuacaccaucgccgacaacggcgacauggugcggguggacguguucugcaagguggacaagaag |
| | | ggcaagaaccaguacuucaucgugcccaucuacgccuggcagguggccgagaacauccugcccgacaucgacugcaagggcuaccggaucg |
| | | acgacuccuacaccuucugcuucucccugcacaaguacgaccugaucgccuuccagaaggacgagaaguccaagguggaguucgccuacua |
| | | caucaacugcgacuccuccaacggccgguucuaccuggccuggcacgacaagggcuccaaggagcagcaguuccggaucuccacccagaac |
| | | cuggugcugauccagaaguaccaggugaacgagcugggcaaggagauccggcccugccggcugaagaagcggccccccgugcgguag |
| 16 | Cas9- | MDGSGGGSPKKKRKVEDKRPAATKKAGQAKKKKGGSGGGAAFKPNPINYILGLDIGIASVGWAMVEIDEEENPIRLIDLGVRVFERAEVPK |
| | Cas9 | TGDSLAMARRLARSVRRLTRRRAHRLLRARRLLKREGVLQAADFDENGLIKSLPNTPWQLRAAALDRKLTPLEWSAVLLHLI KHRGYLSQR |
| | fusion | KNEGETADKELGALLKGVANNAHALQTGDFRTPAELALNKFEKESGHIRNQRGDYSHTFSRKDLQAELILLFEKQKEFGNPHVSGGLKEGI |
| | (mRNA D)- | ETLLMTQRPALSGDAVQKMLGHCTFEPAEPKAAKNTYTAERFIWLTKLNNLRILEQGSERPLTDTERATLMDEPYRKSKLTYAQARKLLGL |
| | amino | EDTAFFKGLRYGKDNAEASTLMEMKAYHAISRALEKEGLKDKKSPLNLSSELQDEIGTAFSLFKTDEDITGRLKDRVQPEILEALLKHISF |
| | acid | DKFVQISLKALRRIVPLMEQGKRYDEACAEIYGDHYGKKNTEEKIYLPPIPADEIRNPVVLRALSQARKVINGVVRRYGSPARIHIETARE |
| | sequence | VGKSFKDRKEIEKRQEENRKDREKAAAKFREYFPNFVGEPKSKDILKLRLYEQQHGKCLYSGKEINLVRLNEKGYVEIDHALPFSRTWDDS |
| | | FNNKVLVLGSENQNKGNQTPYEYFNGKDNSREWQEFKARVETSRFPRSKKQRILLQKFDEDGFKECNLNDTRYVNRFLCQFVADHILLTGK |
| | | GKRRVFASNGQITNLLRGFWGLRKVRAENDRHHALDAVVVACSTVAMQQKITRFVRYKEMNAFDGKTIDKETGKVLHQKTHFPQPWEFFAQ |
| | | EVMIRVFGKPDGKPEFEEADTPEKLRTLLAEKLSSRPEAVHEYVTPLFVSRAPNRKMSGAHKDTLRSAKRFVKHNEKISVKRVWLTEIKLA |
| | | DLENMVNYKNGREIELYEALKARLEAYGGNAKQAFDPKDNPFYKKGGQLVKAVRVEKTQESGVLLNKKNAYTIADNGDMVRVDVFCKVDKK |
| | | GKNQYFIVPIYAWQVAENILPDIDCKGYRIDDSYTFCFSLHKYDLIAFQKDEKSKVEFAYYINCDSSNGRFYLAWHDKGSKEQQFRISTQN |
| | | LVLIQKYQVNELGKEIRPCRLKKRPPVR* |
| 17 | SpyCas9 | GGGAAGCUCAGAAUAAACGCUCAACUUUGGCCGGAUCUGCCACCAUGGACAAGAAGUACUCCAUCGGCCUGGACAUCGGCACCAACUCCGU |
| | (mRNA E)- | GGGCUGGGCCGUGAUCACCGACGAGUACAAGGUGCCCUCCAAGAAGUUCAAGGUGCUGGGCAACACCGACCGGCACUCCAUCAAGAAGAAC |
| | mRNA | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | sequence | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | | GGAGGAGGACAAGAAGCACGAGCGGCACCCCAUCUUCGGCAACAUCGUGGACGAGGUGGCCUACCACGAGAAGUACCCCACCAUCUACCAC |
| | | CUGCGGAAGAAGCUGGUGGACUCCACCGACAAGGCCGACCUGCGGCUGAUCUACCUGGCCCUGGCCCACAUGAUCAAGUUCCGGGGCCACU |
| | | UCCUGAUCGAGGGCGACCUGAACCCCGACAACUCCGACGUGGACAAGCUGUUCAUCCAGCUGGUGCAGACCUACAACCAGCUGUUCGAGGA |
| | | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | | CCGAGGACGCCAAGCUGCAGCUGUCCAAGGACACCUACGACGACGACCUGGACAACCUGCUGGCCCAGAUCGGCGACCAGUACGCCGACCU |
| | | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | | UCCAUGAUCAAGCGGUACGACGAGCACCACCAGGACCUGACCCUGCUGAAGGCCCUGGUGCGGCAGCAGCUGCCCGAGAAGUACAAGGAGA |
| | | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGGAGGACCUGCUGCGGAAGCAGCGGACCUUCGACAACGGCUCCAUC |
| | | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACCGGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGGCGGCUCCCCCAAGAAGAAGCGGAAGGUGUGACU |
| | | AGCACCAGCCUCAAGAACACCCGAAUGGAGUCUCUAAGCUACAUAAUACCAACUUACACUUUACAAAAUGUUGUCCCCCAAAAUGUAGCCA |
| | | UUCGUAUCUGCUCCUAAUAAAAAGAAAGUUUCUUCACAUUCUCUCGAGAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAUCUAG |
| 18 | SpyCas9 | auggacaagaaguacuccaucggccuggacaucggcaccaacuccgugggcugggccgugaucaccgacgaguacaaggugcccuccaaga |
| | (mRNA E)- | aguucaaggugcugggcaacaccgaccggcacuccaucaagaagaaccugaucggcgcccugcuguucgacuccggcgagaccgccgaggc |
| | ORF | cacccggcugaagcggaccgcccggcggcgguacacccggcggaagaaccggaucugcuaccugcaggagaucuucuccaacgagauggcc |
| | sequence | aagguggacgacuccuucuuccaccggcuggaggaguccuuccugguggaggaggacaagaagcacgagcggcaccccaucuucggcaaca |
| | | ucguggacgagguggccuaccacgagaaguaccccaccaucuaccaccugcggaagaagcugguggacuccaccgacaaggccgaccugcg |
| | | gcugaucuaccuggcccuggcccacaugaucaaguuccggggccacuuccugaucgagggcgaccugaaccccgacaacuccgacguggac |
| | | aagcuguucauccagcuggugcagaccuacaaccagcuguucgaggagaaccccaucaacgccuccggcguggacgccaaggccauccugu |
| | | ccgcccggcuguccaagucccggcggcuggagaaccugaucgcccagcugcccggcgagaagaagaacggccuguucggcaaccugaucgc |
| | | ccugucccugggccugacccccaacuucaaguccaacuucgaccuggccgaggacgccaagcugcagcuguccaaggacaccuacgacgac |
| | | gaccuggacaaccugcuggcccagaucggcgaccaguacgccgaccuguuccuggccgccaagaaccuguccgacgccauccugcuguccg |
| | | acauccugcgggugaacaccgagaucaccaaggccccccuguccgccuccaugaucaagcgguacgacgagcaccaccaggaccugacccu |
| | | gcugaaggcccuggugcggcagcagcugcccgagaaguacaaggagaucuucuucgaccaguccaagaacggcuacgccggcuacaucgac |
| | | ggcggcgccucccaggaggaguucuacaaguucaucaagcccauccuggagaagauggacggcaccgaggagcugcuggugaagcugaacc |
| | | gggaggaccugcugcggaagcagcggaccuucgacaacggcuccaucccccaccagauccaccugggcgagcugcacgccauccugcggcg |
| | | gcaggaggacuucuaccccuuccugaaggacaaccgggagaagaucgagaagauccugaccuuccggauccccuacuacgugggcccccug |
| | | gcccggggcaacucccgguucgccuggaugacccggaaguccgaggagaccaucacccccuggaacuucgaggaggugguggacaagggcg |
| | | ccuccgcccaguccuucaucgagcggaugaccaacuucgacaagaaccugcccaacgagaaggugcugcccaagcacucccugcuguacga |
| | | guacuucaccguguacaacgagcugaccaaggugaaguacgugaccgagggcaugcggaagcccgccuuccuguccggcgagcagaagaag |
| | | gccaucguggaccugcuguucaagaccaaccggaaggugaccgugaagcagcugaaggaggacuacuucaagaagaucgagugcuucgacu |
| | | ccguggagaucuccggcguggaggaccgguucaacgccucccugggcaccuaccacgaccugcugaagaucaucaaggacaaggacuuccu |
| | | ggacaacgaggagaacgaggacauccuggaggacaucgugcugacccugacccuguucgaggaccgggagaugaucgaggagcggcugaag |
| | | accuacgcccaccuguucgacgacaaggugaugaagcagcugaagcggcggcgguacaccggcuggggccggcugucccggaagcugauca |
| | | acggcauccgggacaagcaguccggcaagaccauccuggacuuccugaaguccgacggcuucgccaaccggaacuucaugcagcugaucca |
| | | cgacgacucccugaccuucaaggaggacauccagaaggcccagguguccggccagggcgacucccugcacgagcacaucgccaaccuggcc |
| | | ggcucccccgccaucaagaagggcauccugcagaccgugaaggugguggacgagcuggugaaggugaugggccggcacaagcccgagaaca |
| | | ucgugaucgagauggcccgggagaaccagaccacccagaagggccagaagaacucccgggagcggaugaagcggaucgaggagggcaucaa |
| | | ggagcugggcucccagauccugaaggagcaccccguggagaacacccagcugcagaacgagaagcuguaccuguacuaccugcagaacggc |
| | | cgggacauguacguggaccaggagcuggacaucaaccggcuguccgacuacgacguggaccacaucgugccccaguccuuccugaaggacg |
| | | acuccaucgacaacaaggugcugacccgguccgacaagaaccggggcaaguccgacaacgugcccuccgaggagguggugaagaagaugaa |
| | | gaacuacuggcggcagcugcugaacgccaagcugaucacccagcggaaguucgacaaccugaccaaggccgagcggcggggccuguccgag |
| | | cuggacaaggccggcuucaucaagcggcagcugguggagacccggcagaucaccaagcacguggcccagauccuggacucccggaugaaca |
| | | ccaaguacgacgagaacgacaagcugauccgggaggugaaggugaucacccugaaguccaagcugguguccgacuuccggaaggacuucca |
| | | guucuacaaggugcgggagaucaacaacuaccaccacgcccacgacgccuaccugaacgccguggugggcaccgcccugaucaagaaguac |
| | | cccaagcuggaguccgaguucguguacggcgacuacaagguguacgacgugcggaagaugaucgccaaguccgagcaggagaucggcaagg |
| | | ccaccgccaaguacuucuucuacuccaacaucaugaacuucuucaagaccgagaucacccuggccaacggcgagauccggaagcggccccu |
| | | gaucgagaccaacggcgagaccggcgagaucgugugggacaagggccgggacuucgccaccgugcggaaggugcuguccaugccccaggug |
| | | aacaucgugaagaagaccgaggugcagaccggcggcuucuccaaggaguccauccugcccaagcggaacuccgacaagcugaucgcccgga |
| | | agaaggacugggaccccaagaaguacggcggcuucgacucccccaccguggccuacuccgugcuggugguggccaagguggagaagggcaa |
| | | guccaagaagcugaaguccgugaaggagcugcugggcaucaccaucauggagcgguccuccuucgagaagaaccccaucgacuuccuggag |
| | | gccaagggcuacaaggaggugaagaaggaccugaucaucaagcugcccaaguacucccuguucgagcuggagaacggccggaagcggaugc |
| | | uggccuccgccggcgagcugcagaagggcaacgagcuggcccugcccuccaaguacgugaacuuccuguaccuggccucccacuacgagaa |
| | | gcugaagggcucccccgaggacaacgagcagaagcagcuguucguggagcagcacaagcacuaccuggacgagaucaucgagcagaucucc |
| | | gaguucuccaagcgggugauccuggccgacgccaaccuggacaaggugcuguccgccuacaacaagcaccgggacaagcccauccgggagc |
| | | aggccgagaacaucauccaccuguucacccugaccaaccugggcgcccccgccgccuucaaguacuucgacaccaccaucgaccggaagcg |
| | | guacaccuccaccaaggaggugcuggacgccacccugauccaccaguccaucaccggccuguacgagacccggaucgaccugucccagcug |
| | | ggcggcgacggcggcggcucccccaagaagaagcggaagguguga |
| 19 | SpyCas9 | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | (mRNA E)- | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | amino | KLFIQLVQTYNQLFEENPINASGVDAKAILSARLSKSRRLENLIAQLPGEKKNGLFGNLIALSLGLTPNFKSNFDLAEDAKLQLSKDTYDD |
| | acid | DLDNLLAQIGDQYADLFLAAKNLSDAILLSDILRVNTEITKAPLSASMIKRYDEHHQDLTLLKALVRQQLPEKYKEIFFDQSKNGYAGYID |
| | sequence | GGASQEEFYKFIKPILEKMDGTEELLVKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGTYHDLLKIIKDKDFLDNEENEDILEDIVLTLTLFEDREMIEERLK |
| | | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKRYTSTKEVLDATLIHQSITGLYETRIDLSQL |
| | | GGDGGGSPKKKRKV* |
| 20 | Nme2Cas9 | GGGAAGCUCAGAAUAAACGCUCAACUUUGGCCGGAUCUGCCACCAUGACCGGUGCCGCCUUCAAGCCCAACCCCAUCAACUACAUCCUGGG |
| | (mRNA F)- | CCUGGACAUCGGCAUCGCCUCCGUGGGCUGGGCCAUGGUGGAGAUCGACGAGGAGGAGAACCCCAUCCGGCUGAUCGACCUGGGCGUGCGG |
| | mRNA | GUGUUCGAGCGGGCCGAGGUGCCCAAGACCGGCGACUCCCUGGCCAUGGCCCGGCGGCUGGCCCGGUCCGUGCGGCGGCUGACCCGGCGGC |
| | sequence | GGGCCCACCGGCUGCUGCGGGCCCGGCGGCUGCUGAAGCGGGAGGGCGUGCUGCAGGCCGCCGACUUCGACGAGAACGGCCUGAUCAAGUC |
| | | CCUGCCCAACACCCCCUGGCAGCUGCGGGCCGCCGCCCUGGACCGGAAGCUGACCCCCCUGGAGUGGUCCGCCGUGCUGCUGCACCUGAUC |
| | | AAGCACCGGGGCUACCUGUCCCAGCGGAAGAACGAGGGCGAGACCGCCGACAAGGAGCUGGGCGCCCUGCUGAAGGGCGUGGCCAACAACG |
| | | CCCACGCCCUGCAGACCGGCGACUUCCGGACCCCCGCCGAGCUGGCCCUGAACAAGUUCGAGAAGGAGUCCGGCCACAUCCGGAACCAGCG |
| | | GGGCGACUACUCCCACACCUUCUCCCGGAAGGACCUGCAGGCCGAGCUGAUCCUGCUGUUCGAGAAGCAGAAGGAGUUCGGCAACCCCCAC |
| | | GUGUCCGGCGGCCUGAAGGAGGGCAUCGAGACCCUGCUGAUGACCCAGCGGCCCGCCCUGUCCGGCGACGCCGUGCAGAAGAUGCUGGGCC |
| | | ACUGCACCUUCGAGCCCGCCGAGCCCAAGGCCGCCAAGAACACCUACACCGCCGAGCGGUUCAUCUGGCUGACCAAGCUGAACAACCUGCG |
| | | GAUCCUGGAGCAGGGCUCCGAGCGGCCCCUGACCGACACCGAGCGGGCCACCCUGAUGGACGAGCCCUACCGGAAGUCCAAGCUGACCUAC |
| | | GCCCAGGCCCGGAAGCUGCUGGGCCUGGAGGACACCGCCUUCUUCAAGGGCCUGCGGUACGGCAAGGACAACGCCGAGGCCUCCACCCUGA |
| | | UGGAGAUGAAGGCCUACCACGCCAUCUCCCGGGCCCUGGAGAAGGAGGGCCUGAAGGACAAGAAGUCCCCCCUGAACCUGUCCUCCGAGCU |
| | | GCAGGACGAGAUCGGCACCGCCUUCUCCCUGUUCAAGACCGACGAGGACAUCACCGGCCGGCUGAAGGACCGGGUGCAGCCCGAGAUCCUG |
| | | GAGGCCCUGCUGAAGCACAUCUCCUUCGACAAGUUCGUGCAGAUCUCCCUGAAGGCCCUGCGGCGGAUCGUGCCCCUGAUGGAGCAGGGCA |
| | | AGCGGUACGACGAGGCCUGCGCCGAGAUCUACGGCGACCACUACGGCAAGAAGAACACCGAGGAGAAGAUCUACCUGCCCCCCAUCCCCGC |
| | | CGACGAGAUCCGGAACCCCGUGGUGCUGCGGGCCCUGUCCCAGGCCCGGAAGGUGAUCAACGGCGUGGUGCGGCGGUACGGCUCCCCCGCC |
| | | CGGAUCCACAUCGAGACCGCCCGGGAGGUGGGCAAGUCCUUCAAGGACCGGAAGGAGAUCGAGAAGCGGCAGGAGGAGAACCGGAAGGACC |
| | | GGGAGAAGGCCGCCGCCAAGUUCCGGGAGUACUUCCCCAACUUCGUGGGCGAGCCCAAGUCCAAGGACAUCCUGAAGCUGCGGCUGUACGA |
| | | GCAGCAGCACGGCAAGUGCCUGUACUCCGGCAAGGAGAUCAACCUGGUGCGGCUGAACGAGAAGGGCUACGUGGAGAUCGACCACGCCCUG |
| | | CCCUUCUCCCGGACCUGGGACGACUCCUUCAACAACAAGGUGCUGGUGCUGGGCUCCGAGAACCAGAACAAGGGCAACCAGACCCCCUACG |
| | | AGUACUUCAACGGCAAGGACAACUCCCGGGAGUGGCAGGAGUUCAAGGCCCGGGUGGAGACCUCCCGGUUCCCCCGGUCCAAGAAGCAGCG |
| | | GAUCCUGCUGCAGAAGUUCGACGAGGACGGCUUCAAGGAGUGCAACCUGAACGACACCCGGUACGUGAACCGCUUCCUGUGCCAGUUCGUG |
| | | GCCGACCACAUCCUGCUGACCGGCAAGGGCAAGCGGCGGGUGUUCGCCUCCAACGGCCAGAUCACCAACCUGCUGCGGGGCUUCUGGGGCC |
| | | UGCGGAAGGUGCGGGCCGAGAACGACCGGCACCACGCCCUGGACGCCGUGGUGGUGGCCUGCUCCACCGUGGCCAUGCAGCAGAAGAUCAC |
| | | CCGGUUCGUGCGGUACAAGGAGAUGAACGCCUUCGACGGCAAGACCAUCGACAAGGAGACCGGCAAGGUGCUGCACCAGAAGACCCACUUC |
| | | CCCCAGCCCUGGGAGUUCUUCGCCCAGGAGGUGAUGAUCCGGGUGUUCGGCAAGCCCGACGGCAAGCCCGAGUUCGAGGAGGCCGACACCC |
| | | CCGAGAAGCUGCGGACCCUGCUGGCCGAGAAGCUGUCCUCCCGGCCCGAGGCCGUGCACGAGUACGUGACCCCCCUGUUCGUGUCCCGGGC |
| | | CCCCAACCGGAAGAUGUCCGGCGCCCACAAGGACACCCUGCGGUCCGCCAAGCGGUUCGUGAAGCACAACGAGAAGAUCUCCGUGAAGCGG |
| | | GUGUGGCUGACCGAGAUCAAGCUGGCCGACCUGGAGAACAUGGUGAACUACAAGAACGGCCGGGAGAUCGAGCUGUACGAGGCCCUGAAGG |
| | | CCCGGCUGGAGGCCUACGGCGGCAACGCCAAGCAGGCCUUCGACCCCAAGGACAACCCCUUCUACAAGAAGGGCGGCCAGCUGGUGAAGGC |
| | | CGUGCGGGUGGAGAAGACCCAGGAGUCCGGCGUGCUGCUGAACAAGAAGAACGCCUACACCAUCGCCGACAACGGCGACAUGGUGCGGGUG |
| | | GACGUGUUCUGCAAGGUGGACAAGAAGGGCAAGAACCAGUACUUCAUCGUGCCCAUCUACGCCUGGCAGGUGGCCGAGAACAUCCUGCCCG |
| | | ACAUCGACUGCAAGGGCUACCGGAUCGACGACUCCUACACCUUCUGCUUCUCCCUGCACAAGUACGACCUGAUCGCCUUCCAGAAGGACGA |
| | | GAAGUCCAAGGUGGAGUUCGCCUACUACAUCAACUGCGACUCCUCCAACGGCCGGUUCUACCUGGCCUGGCACGACAAGGGCUCCAAGGAG |
| | | CAGCAGUUCCGGAUCUCCACCCAGAACCUGGUGCUGAUCCAGAAGUACCAGGUGAACGAGCUGGGCAAGGAGAUCCGGCCCUGCCGGCUGA |
| | | AGAAGCGGCCCCCCGUGCGGUCCGGAAAGCGGACCGCCGACGGCUCCGAGUUCGAGUCCCCCAAGAAGAAGCGGAAGGUGGAGUAGCUAGC |
| | | ACCAGCCUCAAGAACACCCGAAUGGAGUCUCUAAGCUACAUAAUACCAACUUACACUUUACAAAAUGUUGUCCCCCAAAAUGUAGCCAUUC |
| | | GUAUCUGCUCCUAAUAAAAAGAAAGUUUCUUCACAUUCUCUCGAGAAAAAAAAAAAAUGGAAAAAAAAAAAACGGAAAAAAAAAAAAGGUA |
| | | AAAAAAAAAAAUAUAAAAAAAAAAAACAUAAAAAAAAAAAACGAAAAAAAAAAAACGUAAAAAAAAAAAACUCAAAAAAAAAAAAGAUAAA |
| | | AAAAAAAAACCUAAAAAAAAAAAAUGUAAAAAAAAAAAAGGGAAAAAAAAAAAACGCAAAAAAAAAAAACACAAAAAAAAAAAAUGCAAAA |
| | | AAAAAAAAUCGAAAAAAAAAAAAUCUAAAAAAAAAAAACGAAAAAAAAAAAACCCAAAAAAAAAAAAGACAAAAAAAAAAAAUAGAAAAAA |
| | | AAAAAAGUUAAAAAA |
| 21 | Nme2Ca9 | augaccggugccgccuucaagcccaaccccaucaacuacauccugggccuggacaucggcaucgccuccgugggcugggccaugguggaga |
| | (mRNA F)- | ucgacgaggaggagaaccccauccggcugaucgaccugggcgugcggguguucgagcgggccgaggugcccaagaccggcgacucccuggc |
| | ORF | cauggcccggcggcuggcccgguccgugcggcggcugacccgggcggcggcccaccggcugcugcggcgcccggcggcugcugaaggggag |
| | sequence | ggcgugcugcaggccgccgacuucgacgagaacggccugaucaagucccugcccaacacccccuggcagcugcgggccgccgcccuggacc |
| | | ggaagcugaccccccuggagugguccgccgugcugcugcaccugaucaagcaccggggcuaccugucccagcggaagaacgagggcgagac |
| | | cgccgacaaggagcugggcgcccugcugaagggcguggccaacaacgcccacgcccugcagaccggcgacuuccggacccccgccgagcug |
| | | gcccugaacaaguucgagaaggaguccggccacauccggaaccagcggggcgacuacucccacaccuucucccggaaggaccugcaggccg |
| | | agcugauccugcuguucgagaagcagaaggaguucggcaacccccacguguccggcggccugaaggagggcaucgagacccugcugaugac |
| | | ccagcggcccgcccuguccggcgacgccgugcagaagaugcugggccacugcaccuucgagcccgccgagcccaaggccgccaagaacacc |
| | | uacaccgccgagcgguucaucuggcugaccaagcugaacaaccugcggauccuggagcagggcuccgagcggccccugaccgacaccgagc |
| | | gggccacccugauggacgagcccuaccggaaguccaagcugaccuacgcccaggcccggaagcugcuggcgccuggaggacaccgccuuuu |
| | | caagggccugcgguacggcaaggacaacgccgaggccuccacccugauggagaugaaggccuaccacgccaucucccgggcccuggagaag |
| | | gagggccugaaggacaagaaguccccccugaaccuguccuccgagcugcaggacgagaucggcaccgccuucucccuguucaagaccgacg |
| | | aggacaucaccggccggcugaaggaccgggugcagcccgagauccuggaggcccugcugaagcacaucuccuucgacaaguucgugcagau |
| | | cucccugaaggcccugcggcggaucgugccccugauggagcagggcaagcgguacgacgaggccugcgccgagaucuacggcgaccacuac |
| | | ggcaagaagaacaccgaggagaagaucuaccugccccccauccccgccgacgagauccggaaccccguggugcugcgggcccugucccagg |
| | | cccggaaggugaucaacggcguggugcggcgguacggcucccccgcccggauccacaucgagaccgcccgggaggugggcaaguccuucaa |
| | | ggaccggaaggagaucgagaagcggcaggaggagaaccggaaggaccgggagaaggccgccgccaaguuccgggaguacuuccccaacuuc |
| | | gugggcgagcccaaguccaaggacauccugaagcugcggcuguacgagcagcagcacggcaagugccuguacuccggcaaggagaucaacc |
| | | uggugcggcugaacgagaagggcuacguggagaucgaccacgcccugcccuucucccggaccugggacgacuccuucaacaacaaggugcu |
| | | ggugcugggcuccgagaaccagaacaagggcaaccagacccccuacgaguacuucaacggcaaggacaacucccgggaguggcaggaguuc |
| | | aaggcccggguggagaccucccgguucccccgguccaagaagcagcggauccugcugcagaaguucgacgaggacggcuucaaggagugca |
| | | accugaacgacacccgguacgugaaccgcuuccugugccaguucguggccgaccacauccugcugaccggcaagggcaaggcggcgguguu |
| | | cgccuccaacggccagaucaccaaccugcugcggggcuucugcgggccugcggaagguggggccgagaacgaccggcaccacgcccuggac |
| | | gccguggugguggccugcuccaccguggccaugcagcagaagaucacccgguucgugcgguacaaggagaugaacgccuucgacggcaaga |
| | | ccaucgacaaggagaccggcaaggugcugcaccagaagacccacuucccccagcccugggaguucuucgcccaggaggugaugauccgggu |
| | | guucggcaagcccgacggcaagcccgaguucgaggaggccgacacccccgagaagcugcggacccugcuggccgagaagcuguccucccgg |
| | | cccgaggccgugcacgaguacgugaccccccuguucgugucccgggcccccaaccggaagauguccggcgcccacaaggacacccugcggu |
| | | ccgccaagcgguucgugaagcacaacgagaagaucuccgugaagcggguguggcugaccgagaucaagcuggccgaccuggagaacauggu |
| | | gaacuacaagaacggccgggagaucgagcuguacgaggcccugaaggcccggcuggaggccuacgggcgcaacgccaagcaggccuucgac |
| | | cccaaggacaaccccuucuacaagaagggcggccagcuggugaaggccgugcggguggagaagacccaggaguccggcgugcugcugaaca |
| | | agaagaacgccuacaccaucgccgacaacggcgacauggugcggguggacguguucugcaagguggacaagaagggcaagaaccaguacuu |
| | | caucgugcccaucuacgccuggcagguggccgagaacauccugcccgacaucgacugcaagggcuaccggaucgacgacuccuacaccuuc |
| | | ugcuucucccugcacaaguacgaccugaucgccuuccagaaggacgagaaguccaagguggaguucgccuacuacaucaacugcgacuccu |
| | | ccaacggccgguucuaccuggccuggcacgacaagggcuccaaggagcagcaguuccggaucuccacccagaaccuggugcugauccagaa |
| | | guaccaggugaacgagcugggcaaggagauccggcccugccggcugaagaagcggccccccgugcgguccggaaagcggaccgccgacggc |
| | | uccgaguucgagucccccaagaagaagcggaagguggaguag |
| 22 | Nme2Cas9 | MTGAAFKPNPINYILGLDIGIASVGWAMVEIDEEENPIRLIDLGVRVFERAEVPKTGDSLAMARRLARSVRRLTRRRAHRLLRARRLLKRE |
| | (mRNA F)- | GVLQAADFDENGLIKSLPNTPWQLRAAALDRKLTPLEWSAVLLHLIKHRGYLSQRKNEGETADKELGALLKGVANNAHALQTGDFRTPAEL |
| | amino | ALNKFEKESGHIRNQRGDYSHTFSRKDLQAELILLFEKQKEFGNPHVSGGLKEGIETLLMTQRPALSGDAVQKMLGHCTFEPAEPKAAKNT |
| | acid | YTAERFIWLTKLNNLRILEQGSERPLTDTERATLMDEPYRKSKLTYAQARKLLGLEDTAFFKGLRYGKDNAEASTLMEMKAYHAISRALEK |
| | sequence | EGLKDKKSPLNLSSELQDEIGTAFSLFKTDEDITGRLKDRVQPEILEALLKHISFDKFVQISLKALRRIVPLMEQGKRYDEACAEIYGDHY |
| | | GKKNTEEKIYLPPIPADEIRNPVVLRALSQARKVINGVVRRYGSPARIHIETAREVGKSFKDRKEIEKRQEENRKDREKAAAKFREYFPNF |
| | | VGEPKSKDILKLRLYEQQHGKCLYSGKEINLVRLNEKGYVEIDHALPFSRTWDDSFNNKVLVLGSENQNKGNQTPYEYFNGKDNSREWQEF |
| | | KARVETSRFPRSKKQRILLQKFDEDGFKECNLNDTRYVNRFLCQFVADHILLTGKGKRRVFASNGQITNLLRGFWGLRKVRAENDRHHALD |
| | | AVVVACSTVAMQQKITRFVRYKEMNAFDGKTIDKETGKVLHQKTHFPQPWEFFAQEVMIRVFGKPDGKPEFEEADTPEKLRTLLAEKLSSR |
| | | PEAVHEYVTPLFVSRAPNRKMSGAHKDTLRSAKRFVKHNEKISVKRVWLTEIKLADLENMVNYKNGREIELYEALKARLEAYGGNAKQAFD |
| | | PKDNPFYKKGGQLVKAVRVEKTQESGVLLNKKNAYTIADNGDMVRVDVFCKVDKKGKNQYFIVPIYAWQVAENILPDIDCKGYRIDDSYTF |
| | | CFSLHKYDLIAFQKDEKSKVEFAYYINCDSSNGRFYLAWHDKGSKEQQFRISTQNLVLIQKYQVNELGKEIRPCRLKKRPPVRSGKRTADG |
| | | SEFESPKKKRKVE* |
| 23 | Nme2Cas9 | GGGAAGCUCAGAAUAAACGCUCAACUUUGGCCGGAUCUGCCACCAUGGACGGCUCCGGGGGGGGCUCCCCCAAGAAGAAGCGGAAGGUGGA |
| | Intein | GGACAAGCGGCCCGCCGCCACCAAGAAGGCCGGCCAGGCCAAGAAGAAGAAGGGCGGCUCCGGGGGGGGCGCCGCCUUCAAGCCCAACCCC |
| | (mRNA G)- | AUCAACUACAUCCUGGGCCUGGACAUCGGCAUCGCCUCCGUGGGCUGGGCCAUGGUGGAGAUCGACGAGGAGGAGAACCCCAUCCGGCUGA |
| | mRNA | UCGACCUGGGCGUGCGGGUGUUCGAGCGGGCCGAGGUGCCCAAGACCGGCGACUCCCUGGCCAUGGCCCGGCGGCUGGCCCGGUCCGUGCG |
| | sequence | GCGGCUGACCCGGCGGCGGGCCCACCGGCUGCUGCGGGCCCGGCGGCUGCUGAAGCGGGAGGGCGUGCUGCAGGCCGCCGACUUCGACGAG |
| | | AACGGCCUGAUCAAGUCCCUGCCCAACACCCCCUGGCAGCUGCGGGCCGCCGCCCUGGACCGGAAGCUGACCCCCCUGGAGUGGUCCGCCG |
| | | UGCUGCUGCACCUGAUCAAGCACCGGGGCUACCUGUCCCAGCGGAAGAACGAGGGCGAGACCGCCGACAAGGAGCUGGGCGCCCUGCUGAA |
| | | GGGCGUGGCCAACAACGCCCACGCCCUGCAGACCGGCGACUUCCGGACCCCCGCCGAGCUGGCCCUGAACAAGUUCGAGAAGGAGUCCGGC |
| | | CACAUCCGGAACCAGCGGGGCGACUACUCCCACACCUUCUCCCGGAAGGACCUGCAGGCCGAGCUGAUCCUGCUGUUCGAGAAGCAGAAGG |
| | | AGUUCGGCAACCCCCACGUGUCCGGCGGCCUGAAGGAGGGCAUCGAGACCCUGCUGAUGACCCAGCGGCCCGCCCUGUCCGGCGACGCCGU |
| | | GCAGAAGAUGCUGGGCCACUGCACCUUCGAGCCCGCCGAGCCCAAGGCCGCCAAGAACACCUACACCGCCGAGCGGUUCAUCUGGCUGACC |
| | | AAGCUGAACAACCUGCGGAUCCUGGAGCAGGGCUCCGAGCGGCCCCUGACCGACACCGAGCGGGCCACCCUGAUGGACGAGCCCUACCGGA |
| | | AGUCCAAGCUGACCUACGCCCAGGCCCGGAAGCUGCUGGGCCUGGAGGACACCGCCUUCUUCAAGGGCCUGCGGUACGGCAAGGACAACGC |
| | | CGAGGCCUCCACCCUGAUGGAGAUGAAGGCCUACCACGCCAUCUCCCGGGCCCUGGAGAAGGAGGGCCUGAAGGACAAGAAGUCCCCCCUG |
| | | AACCUGUCCUCCGAGCUGCAGGACGAGAUCGGCACCGCCUUCUCCCUGUUCAAGACCGACGAGGACAUCACCGGCCGGCUGAAGGACCGGG |
| | | UGCAGCCCGAGAUCCUGGAGGCCCUGCUGAAGCACAUCUCCUUCGACAAGUUCGUGCAGAUCUCCCUGAAGGCCCUGCGGCGGAUCGUGCC |
| | | CCUGAUGGAGCAGGGCAAGCGGUACGACGAGGCCUGCGCCGAGAUCUACGGCGACCACUACGGCAAGAAGAACACCGAGGAGAAGAUCUAC |
| | | CUGCCCCCCAUCCCCGCCGACGAGAUCCGGAACCCCGUGGUGCUGCGGGCCCUGUCCCAGGCCCGGAAGGUGAUCAACGGCGUGGUGCGGC |
| | | GGUACGGCUCCCCCGCCCGGAUCCACAUCGAGACCGCCCGGGAGGUGGGCAAGUCCUUCAAGGACCGGAAGGAGAUCGAGAAGCGGCAGGA |
| | | GGAGAACCGGAAGGACCGGGAGAAGGCCGCCGCCAAGUUCCGGGAGUACUUCCCCAACUUCGUGGGCGAGCCCAAGUCCAAGGACAUCCUG |
| | | AAGCUGCGGCUGUACGAGCAGCAGCACGGCAAGUGCCUGUACUCCGGCAAGGAGAUCAACCUGGUGCGGCUGAACGAGAAGGGCUACGUGG |
| | | AGAUCGACCACGCCCUGCCCUUCUCCCGGACCUGGGACGACUCCUUCAACAACAAGGUGCUGGUGCUGGGCUCCGAGAACCAGAACAAGGG |
| | | CAACCAGACCCCCUACGAGUACUUCAACGGCAAGGACAACUCCCGGGAGUGGCAGGAGUUCAAGGCCCGGGUGGAGACCUCCCGGUUCCCC |
| | | CGGUCCAAGAAGCAGCGGAUCCUGCUGCAGAAGUUCGACGAGGACGGCUUCAAGGAGUGCAACCUGAACGACACCCGGUACGUGAACCGGU |
| | | UCCUGUGCCAGUUCGUGGCCGACCACAUCCUGCUGACCGGCAAGGGCAAGCGGCGGGUGUUCGCCUCCAACGGCCAGAUCACCAACCUGCU |
| | | GCGGGGCUUCUGGGGCCUGCGGAAGGUGCGGGCCGAGAACGACCGGCACCACGCCCUGGACGCCGUGGUGGUGGCCUGCUCCACCGUGGCC |
| | | AUGCAGCAGAAGAUCACCCGGUUCGUGCGGUACAAGGAGAUGAACGCCUUCGACGGCAAGACCAUCGACAAGGAGACCGGCAAGGUGCUGC |
| | | ACCAGAAGACCCACUUCCCCCAGCCCUGGGAGUUCUUCGCCCAGGAGGUGAUGAUCCGGGUGUUCGGCAAGCCCGACGGCAAGCCCGAGUU |
| | | CGAGGAGGCCGACACCCCCGAGAAGCUGCGGACCCUGCUGGCCGAGAAGCUGUCCUCCCGGCCCGAGGCCGUGCACGAGUACGUGACCCCC |
| | | CUGUUCGUGUCCCGGGCCCCCAACCGGAAGAUGUCCGGCGCCCACAAGGACACCCUGCGGUCCGCCAAGCGGUUCGUGAAGCACAACGAGA |
| | | AGAUCUCCGUGAAGCGGGUGUGGCUGACCGAGAUCAAGCUGGCCGACCUGGAGAACAUGGUGAACUACAAGAACGGCCGGGAGAUCGAGCU |
| | | GUACGAGGCCCUGAAGGCCCGGCUGGAGGCCUACGGCGGCAACGCCAAGCAGGCCUUCGACCCCAAGGACAACCCCUUCUACAAGAAGGGC |
| | | GGCCAGCUGGUGAAGGCCGUGCGGGUGGAGAAGACCCAGGAGUCCGGCGUGCUGCUGAACAAGAAGAACGCCUACACCAUCGCCGACAACG |
| | | GCGACAUGGUGCGGGUGGACGUGUUCUGCAAGGUGGACAAGAAGGGCAAGAACCAGUACUUCAUCGUGCCCAUCUACGCCUGGCAGGUGGC |
| | | CGAGAACAUCCUGCCCGACAUCGACUGCAAGGGCUACCGGAUCGACGACUCCUACACCUUCUGCUUCUCCCUGCACAAGUACGACCUGAUC |
| | | GCCUUCCAGAAGGACGAGAAGUCCAAGGUGGAGUUCGCCUACUACAUCAACUGCGACUCCUCCAACGGCCGGUUCUACCUGGCCUGGCACG |
| | | ACAAGGGCUCCAAGGAGCAGCAGUUCCGGAUCUCCACCCAGAACCUGGUGCUGAUCCAGAAGUACCAGGUGAACGAGCUGGGCAAGGAGAU |
| | | CCGGCCCUGCCGGCUGAAGAAGCGGCCCCCCGUGCGGGGCGCCCCCGAGUCCGCCACCGAGUCCGGCGGCACCUCCACCGAGUCCGAGGGC |
| | | UCCGCCGGCACCUCCACCGAGUCCGAGGGCUCCGCCGGCUCCGCCGGCUCCACCUCCGGCGGCUCCGGCGGCGGCUCCGGCGGCUCCGGCU |
| | | CCGCCGGCACCUCCACCGAGUCCGAGGGCUCCGCCGGCACCUCCGAGUCCGCCACCGAGUCCGGCGGCACCUCCACCGAGUCCGAGGGCUC |
| | | CUCCUCCCGGGGCAAGUGCCUGUCCUACGAGACCGAGAUCCUGACCGUGGAGUACGGCCUGCUGCCCAUCGUGGUGGUGGUGGAGAAGCGG |
| | | AUCGAGUGCACCGUGUACUCCGUGGACAACAACGGCAACAUCUACACCCAGCCCGUGGCCCAGUGGCACGACCGGGGCGAGCAGGAGGUGU |
| | | UCGAGUACUGCCUGGAGGACGGCUCCCUGAUCCGGGCCACCAAGGACCACAAGUUCAUGACCGUGGACGGCCAGAUGCUGCCCAUCGACGA |
| | | GAUCUUCGAGCGGGAGCUGGACCUGAUGCGGGUGGACAACCUGCCCAACUAGCUAGCACCAGCCUCAAGAACACCCGAAUGGAGUCUCUAA |
| | | GCUACAUAAUACCAACUUACACUUUACAAAAUGUUGUCCCCCAAAAUGUAGCCAUUCGUAUCUGCUCCUAAUAAAAAGAAAGUUUCUUCAC |
| | | AUUCUCUCGAGAAAAAAAAAAAAUGGAAAAAAAAAAAACGGAAAAAAAAAAAAGGUAAAAAAAAAAAAUAUAAAAAAAAAAAACAUAAAAA |
| | | AAAAAAACGAAAAAAAAAAAACGUAAAAAAAAAAAACUCAAAAAAAAAAAGAUAAAAAAAAAAAACCUAAAAAAAAAAAAUGUAAAAAAAA |
| | | AAAAGGGAAAAAAAAAAAACGCAAAAAAAAAAAACACAAAAAAAAAAAAUGCAAAAAAAAAAAAUCGAAAAAAAAAAAAUCUAAAAAAAAA |
| | | AAACGAAAAAAAAAAAACCCAAAAAAAAAAAAGACAAAAAAAAAAAAUAGAAAAAAAAAAAGUUAAAAAAAAAAAACUGAAAAAAAAAAAA |
| | | UUUAAAAAAAAAAAAUCUAG |
| 24 | Nme2Cas9 | augGACGGCUCCGGCGGCGGCUCCCCCAAGAAGAAGCGGAAGGUGGAGGACAAGCGGCCCGCCGCCACCAAGAAGGCCGGCCAGGCCAAGA |
| | Intein | AGAAGAAGGGCGGCUCCGGCGGCGGCgccgccuucaagcccaaccccaucaacuacauccugggccuggacaucggcaucgccuccguggg |
| | (mRNA G)- | cugggccaugguggagaucgacgaggaggagaaccccauccggcugaucgaccugggcgugcgcgguguucgaggggccgaggugcccaag |
| | ORF | accggcgacucccuggccauggcccggcggcuggcccgguccguggcgggccugacccggcggcgcggcccaccggcugcuggggcccggc |
| | sequence | ggcugcugaagcgggagggcgugcugcaggccgccgacuucgacgagaacggccugaucaagucccugcccaacacccccuggcagcugcg |
| | | ggccgccgcccuggaccggaagcugaccccccuggagugguccgccgugcugcugcaccugaucaagcaccggggcuaccugucccagcgg |
| | | aagaacgagggcgagaccgccgacaaggagcugggcgcccugcugaagggcguggccaacaacgcccacgcccugcagaccggcgacuucc |
| | | ggacccccgccgagcuggcccugaacaaguucgagaaggaguccggccacauccggaaccagcggggcgacuacucccacaccuucucccg |
| | | gaaggaccugcaggccgagcugauccugcuguucgagaagcagaaggaguucggcaacccccacguguccggcggccugaaggagggcauc |
| | | gagacccugcugaugacccagcggcccgcccuguccggcgacgccgugcagaagaugcugggccacugcaccuucgagcccgccgagccca |
| | | aggccgccaagaacaccuacaccgccgagcgguucaucuggcugaccaagcugaacaaccugcggauccuggagcagggcuccgagcggcc |
| | | ccugaccgacaccgagcgggccacccugauggacgagcccuaccggaaguccaagcugaccuacgcccaggcccggaagcugcugggccug |
| | | gaggacaccgccuucuucaagggccugcgguacggcaaggacaacgccgaggccuccacccugauggagaugaaggccuaccacgccaucu |
| | | cccgggcccuggagaaggagggccugaaggacaagaaguccccccugaaccuguccuccgagcugcaggacgagaucggcaccgccuucuc |
| | | ccuguucaagaccgacgaggacaucaccggccggcugaaggaccgggugcagcccgagauccuggaggcccugcugaagcacaucuccuuc |
| | | gacaaguucgugcagaucucccugaaggcccugcggcggaucgugccccugauggagcagggcaagcgguacgacgaggccugcgccgaga |
| | | ucuacggcgaccacuacggcaagaagaacaccgaggagaagaucuaccugccccccauccccgccgacgagauccggaaccccguggugcu |
| | | gcgggcccugucccaggcccggaaggugaucaacggcguggugcggcgguacggcucccccgcccggauccacaucgagaccgcccgggag |
| | | gugggcaaguccuucaaggaccggaaggagaucgagaagcggcaggaggagaaccggaaggaccgggagaaggccgccgccaaguuccggg |
| | | aguacuuccccaacuucgugggcgagcccaaguccaaggacauccugaagcugcggcuguacgagcagcagcacggcaagugccuguacuc |
| | | cggcaaggagaucaaccuggugcggcugaacgagaagggcuacguggagaucgaccacgcccugcccuucucccggaccugggacgacucc |
| | | uucaacaacaaggugcuggugcugggcuccgagaaccagaacaagggcaaccagacccccuacgaguacuucaacggcaaggacaacuccc |
| | | gggaguggcaggaguucaaggcccggguggagaccucccgguucccccgguccaagaagcagcggauccugcugcagaaguucgacgagga |
| | | cggcuucaaggagugcaaccugaacgacacccgguacgugaaccgguuccugugccaguucguggccgaccacauccugcugaccggcaag |
| | | ggcaagcggcggguguucgccuccaacggccagaucaccaaccugcugcggggcuucuggggccugcggaaggugcgggccgagaacgacc |
| | | ggcaccacgcccuggacgccguggugguggccugcuccaccguggccaugcagcagaagaucacccgguucgugcgguacaaggagaugaa |
| | | cgccuucgacggcaagaccaucgacaaggagaccggcaaggugcugcaccagaagacccacuucccccagcccugggaguucuucgcccag |
| | | gaggugaugauccggguguucggcaagcccgacggcaagcccgaguucgaggaggccgacacccccgagaagcugcggacccugcuggccg |
| | | agaagcuguccucccggcccgaggccgugcacgaguacgugaccccccuguucgugucccgggcccccaaccggaagauguccggcgccca |
| | | caaggacacccugcgguccgccaagcgguucgugaagcacaacgagaagaucuccgugaagcggguguggcugaccgagaucaagcuggcc |
| | | gaccuggagaacauggugaacuacaagaacggccgggagaucgagcuguacgaggcccugaaggcccggcuggaggccuacggcggcaacg |
| | | ccaagcaggccuucgaccccaaggacaaccccuucuacaagaagggcggccagcuggugaaggccgugcggguggagaagacccaggaguc |
| | | cggcgugcugcugaacaagaagaacgccuacaccaucgccgacaacggcgacauggugcggguggacguguucugcaagguggacaagaag |
| | | ggcaagaaccaguacuucaucgugcccaucuacgccuggcagguggccgagaacauccugcccgacaucgacugcaagggcuaccggaucg |
| | | acgacuccuacaccuucugcuucucccugcacaaguacgaccugaucgccuuccagaaggacgagaaguccaagguggaguucgccuacua |
| | | caucaacugcgacuccuccaacggccgguucuaccuggccuggcacgacaagggcuccaaggagcagcaguuccggaucuccacccagaac |
| | | cuggugcugauccagaaguaccaggugaacgagcugggcaaggagauccggcccugccggcugaagaagcggccccccgugcggGGCGCCC |
| | | CCGAGUCCGCCACCGAGUCCGGCGGCACCUCCACCGAGUCCGAGGGCUCCGCCGGCACCUCCACCGAGUCCGAGGGCUCCGCCGGCUCCGC |
| | | CGGCUCCACCUCCGGCGGCUCCGGCGGCGGCUCCGGCGGCUCCGGCUCCGCCGGCACCUCCACCGAGUCCGAGGGCUCCGCCGGCACCUCC |
| | | GAGUCCGCCACCGAGUCCGGCGGCACCUCCACCGAGUCCGAGGGCUCCUCCUCCCGGGGCAAGUGCCUGUCCUACGAGACCGAGAUCCUGA |
| | | CCGUGGAGUACGGCCUGCUGCCCAUCGUGGUGGUGGUGGAGAAGCGGAUCGAGUGCACCGUGUACUCCGUGGACAACAACGGCAACAUCUA |
| | | CACCCAGCCCGUGGCCCAGUGGCACGACCGGGGCGAGCAGGAGGUGUUCGAGUACUGCCUGGAGGACGGCUCCCUGAUCCGGGCCACCAAG |
| | | GACCACAAGUUCAUGACCGUGGACGGCCAGAUGCUGCCCAUCGACGAGAUCUUCGAGCGGGAGCUGGACCUGAUGCGGGUGGACAACCUGC |
| | | CCAACuag |
| 25 | Nme2Cas9 | MDGSGGGSPKKKRKVEDKRPAATKKAGQAKKKKGGSGGGAAFKPNPINYILGLDIGIASVGWAMVEIDEEENPIRLIDLGVRVFERAEVPK |
| | Intein | TGDSLAMARRLARSVRRLTRRRAHRLLRARRLLKREGVLQAADFDENGLIKSLPNTPWQLRAAALDRKLTPLEWSAVLLHLIKHRGYLSQR |
| | (mRNA G)- | KNEGETADKELGALLKGVANNAHALQTGDFRTPAELALNKFEKESGHIRNQRGDYSHTFSRKDLQAELILLFEKQKEFGNPHVSGGLKEGI |
| | amino | ETLLMTQRPALSGDAVQKMLGHCTFEPAEPKAAKNTYTAERFIWLTKLNNLRILEQGSERPLTDTERATLMDEPYRKSKLTYAQARKLLGL |
| | acid | EDTAFFKGLRYGKDNAEASTLMEMKAYHAISRALEKEGLKDKKSPLNLSSELQDEIGTAFSLFKTDEDITGRLKDRVQPEILEALLKHISF |
| | sequence | DKFVQISLKALRRIVPLMEQGKRYDEACAEIYGDHYGKKNTEEKIYLPPIPADEIRNPVVLRALSQARKVINGVVRRYGSPARIHIETARE |
| | | VGKSFKDRKEIEKRQEENRKDREKAAAKFREYFPNFVGEPKSKDILKLRLYEQQHGKCLYSGKEINLVRLNEKGYVEIDHALPFSRTWDDS |
| | | FNNKVLVLGSENQNKGNQTPYEYFNGKDNSREWQEFKARVETSRFPRSKKQRILLQKFDEDGFKECNLNDTRYVNRFLCQFVADHILLTGK |
| | | GKRRVFASNGQITNLLRGFWGLRKVRAENDRHHALDAVVVACSTVAMQQKITRFVRYKEMNAFDGKTIDKETGKVLHQKTHFPQPWEFFAQ |
| | | EVMIRVFGKPDGKPEFEEADTPEKLRTLLAEKLSSRPEAVHEYVTPLFVSRAPNRKMSGAHKDTLRSAKRFVKHNEKISVKRVWLTEIKLA |
| | | DLENMVNYKNGREIELYEALKARLEAYGGNAKQAFDPKDNPFYKKGGQLVKAVRVEKTQESGVLLNKKNAYTIADNGDMVRVDVFCKVDKK |
| | | GKNQYFIVPIYAWQVAENILPDIDCKGYRIDDSYTFCFSLHKYDLIAFQKDEKSKVEFAYYINCDSSNGRFYLAWHDKGSKEQQFRISTQN |
| | | LVLIQKYQVNELGKEIRPCRLKKRPPVRGAPESATESGGTSTESEGSAGTSTESEGSAGSAGSTSGGSGGGSGGSGSAGTSTESEGSAGTS |
| | | ESATESGGTSTESEGSSSRGKCLSYETEILTVEYGLLPIVVVVEKRIECTVYSVDNNGNIYTQPVAQWHDRGEQEVFEYCLEDGSLIRATK |
| | | DHKFMTVDGQMLPIDEIFERELDLMRVDNLPN* |
| 26 | Spy- | GGGAAGCUCAGAAUAAACGCUCAACUUUGGCCGGAUCUGCCACCAUGAUCAAGAUCGCCACCCGGAAGUACCUGGGCAAGCAGAACGUGUA |
| | R1333K | CGACAUCGGCGUGGAGCGGGACCACAACUUCGCCCUGAAGAACGGCUUCAUCGCCUCCAACUGCUGGGAGGGCUCCGGCGACAAGAAGUAC |
| | Cas9 | UCCAUCGGCCUGGACAUCGGCACCAACUCCGUGGGCUGGGCCGUGAUCACCGACGAGUACAAGGUGCCCUCCAAGAAGUUCAAGGUGCUGG |
| | Intein | GCAACACCGACCGGCACUCCAUCAAGAAGAACCUGAUCGGCGCCCUGCUGUUCGACUCCGGCGAGACCGCCGAGGCCACCCGGCUGAAGCG |
| | (mRNA H)- | GACCGCCCGGCGGCGGUACACCCGGCGGAAGAACCGGAUCUGCUACCUGCAGGAGAUCUUCUCCAACGAGAUGGCCAAGGUGGACGACUCC |
| | mRNA | UUCUUCCACCGGCUGGAGGAGUCCUUCCUGGUGGAGGAGGACAAGAAGCACGAGCGGCACCCCAUCUUCGGCAACAUCGUGGACGAGGUGG |
| | sequence | CCUACCACGAGAAGUACCCCACCAUCUACCACCUGCGGAAGAAGCUGGUGGACUCCACCGACAAGGCCGACCUGCGGCUGAUCUACCUGGC |
| | | CCUGGCCCACAUGAUCAAGUUCCGGGGCCACUUCCUGAUCGAGGGCGACCUGAACCCCGACAACUCCGACGUGGACAAGCUGUUCAUCCAG |
| | | CUGGUGCAGACCUACAACCAGCUGUUCGAGGAGAACCCCAUCAACGCCUCCGGCGUGGACGCCAAGGCCAUCCUGUCCGCCCGGCUGUCCA |
| | | AGUCCCGGCGGCUGGAGAACCUGAUCGCCCAGCUGCCCGGCGAGAAGAAGAACGGCCUGUUCGGCAACCUGAUCGCCCUGUCCCUGGGCCU |
| | | GACCCCCAACUUCAAGUCCAACUUCGACCUGGCCGAGGACGCCAAGCUGCAGCUGUCCAAGGACACCUACGACGACGACCUGGACAACCUG |
| | | CUGGCCCAGAUCGGCGACCAGUACGCCGACCUGUUCCUGGCCGCCAAGAACCUGUCCGACGCCAUCCUGCUGUCCGACAUCCUGCGGGUGA |
| | | ACACCGAGAUCACCAAGGCCCCCCUGUCCGCCUCCAUGAUCAAGCGGUACGACGAGCACCACCAGGACCUGACCCUGCUGAAGGCCCUGGU |
| | | GCGGCAGCAGCUGCCCGAGAAGUACAAGGAGAUCUUCUUCGACCAGUCCAAGAACGGCUACGCCGGCUACAUCGACGGCGGCGCCUCCCAG |
| | | GAGGAGUUCUACAAGUUCAUCAAGCCCAUCCUGGAGAAGAUGGACGGCACCGAGGAGCUGCUGGUGAAGCUGAACCGGGAGGACCUGCUGC |
| | | GGAAGCAGCGGACCUUCGACAACGGCUCCAUCCCCCACCAGAUCCACCUGGGCGAGCUGCACGCCAUCCUGCGGCGGCAGGAGGACUUCUA |
| | | CCCCUUCCUGAAGGACAACCGGGAGAAGAUCGAGAAGAUCCUGACCUUCCGGAUCCCCUACUACGUGGGCCCCCUGGCCCGGGGCAACUCC |
| | | CGGUUCGCCUGGAUGACCCGGAAGUCCGAGGAGACCAUCACCCCCUGGAACUUCGAGGAGGUGGUGGACAAGGGCGCCUCCGCCCAGUCCU |
| | | UCAUCGAGCGGAUGACCAACUUCGACAAGAACCUGCCCAACGAGAAGGUGCUGCCCAAGCACUCCCUGCUGUACGAGUACUUCACCGUGUA |
| | | CAACGAGCUGACCAAGGUGAAGUACGUGACCGAGGGCAUGCGGAAGCCCGCCUUCCUGUCCGGCGAGCAGAAGAAGGCCAUCGUGGACCUG |
| | | CUGUUCAAGACCAACCGGAAGGUGACCGUGAAGCAGCUGAAGGAGGACUACUUCAAGAAGAUCGAGUGCUUCGACUCCGUGGAGAUCUCCG |
| | | GCGUGGAGGACCGGUUCAACGCCUCCCUGGGCACCUACCACGACCUGCUGAAGAUCAUCAAGGACAAGGACUUCCUGGACAACGAGGAGAA |
| | | CGAGGACAUCCUGGAGGACAUCGUGCUGACCCUGACCCUGUUCGAGGACCGGGAGAUGAUCGAGGAGCGGCUGAAGACCUACGCCCACCUG |
| | | UUCGACGACAAGGUGAUGAAGCAGCUGAAGCGGCGGCGGUACACCGGCUGGGGCCGGCUGUCCCGGAAGCUGAUCAACGGCAUCCGGGACA |
| | | AGCAGUCCGGCAAGACCAUCCUGGACUUCCUGAAGUCCGACGGCUUCGCCAACCGGAACUUCAUGCAGCUGAUCCACGACGACUCCCUGAC |
| | | CUUCAAGGAGGACAUCCAGAAGGCCCAGGUGUCCGGCCAGGGCGACUCCCUGCACGAGCACAUCGCCAACCUGGCCGGCUCCCCCGCCAUC |
| | | AAGAAGGGCAUCCUGCAGACCGUGAAGGUGGUGGACGAGCUGGUGAAGGUGAUGGGCCGGCACAAGCCCGAGAACAUCGUGAUCGAGAUGG |
| | | CCCGGGAGAACCAGACCACCCAGAAGGGCCAGAAGAACUCCCGGGAGCGGAUGAAGCGGAUCGAGGAGGGCAUCAAGGAGCUGGGCUCCCA |
| | | GAUCCUGAAGGAGCACCCCGUGGAGAACACCCAGCUGCAGAACGAGAAGCUGUACCUGUACUACCUGCAGAACGGCCGGGACAUGUACGUG |
| | | GACCAGGAGCUGGACAUCAACCGGCUGUCCGACUACGACGUGGACCACAUCGUGCCCCAGUCCUUCCUGAAGGACGACUCCAUCGACAACA |
| | | AGGUGCUGACCCGGUCCGACAAGAACCGGGGCAAGUCCGACAACGUGCCCUCCGAGGAGGUGGUGAAGAAGAUGAAGAACUACUGGCGGCA |
| | | GCUGCUGAACGCCAAGCUGAUCACCCAGCGGAAGUUCGACAACCUGACCAAGGCCGAGCGGGGCGGCCUGUCCGAGCUGGACAAGGCCGGC |
| | | UUCAUCAAGCGGCAGCUGGUGGAGACCCGGCAGAUCACCAAGCACGUGGCCCAGAUCCUGGACUCCCGGAUGAACACCAAGUACGACGAGA |
| | | ACGACAAGCUGAUCCGGGAGGUGAAGGUGAUCACCCUGAAGUCCAAGCUGGUGUCCGACUUCCGGAAGGACUUCCAGUUCUACAAGGUGCG |
| | | GGAGAUCAACAACUACCACCACGCCCACGACGCCUACCUGAACGCCGUGGUGGGCACCGCCCUGAUCAAGAAGUACCCCAAGCUGGAGUCC |
| | | GAGUUCGUGUACGGCGACUACAAGGUGUACGACGUGCGGAAGAUGAUCGCCAAGUCCGAGCAGGAGAUCGGCAAGGCCACCGCCAAGUACU |
| | | UCUUCUACUCCAACAUCAUGAACUUCUUCAAGACCGAGAUCACCCUGGCCAACGGCGAGAUCCGGAAGCGGCCCCUGAUCGAGACCAACGG |
| | | CGAGACCGGCGAGAUCGUGUGGGACAAGGGCCGGGACUUCGCCACCGUGCGGAAGGUGCUGUCCAUGCCCCAGGUGAACAUCGUGAAGAAG |
| | | ACCGAGGUGCAGACCGGCGGCUUCUCCAAGGAGUCCAUCCUGCCCAAGCGGAACUCCGACAAGCUGAUCGCCCGGAAGAAGGACUGGGACC |
| | | CCAAGAAGUACGGCGGCUUCGACUCCCCCACCGUGGCCUACUCCGUGCUGGUGGUGGCCAAGGUGGAGAAGGGCAAGUCCAAGAAGCUGAA |
| | | GUCCGUGAAGGAGCUGCUGGGCAUCACCAUCAUGGAGCGGUCCUCCUUCGAGAAGAACCCCAUCGACUUCCUGGAGGCCAAGGGCUACAAG |
| | | GAGGUGAAGAAGGACCUGAUCAUCAAGCUGCCCAAGUACUCCCUGUUCGAGCUGGAGAACGGCCGGAAGCGGAUGCUGGCCUCCGCCGGCG |
| | | AGCUGCAGAAGGGCAACGAGCUGGCCCUGCCCUCCAAGUACGUGAACUUCCUGUACCUGGCCUCCCACUACGAGAAGCUGAAGGGCUCCCC |
| | | CGAGGACAACGAGCAGAAGCAGCUGUUCGUGGAGCAGCACAAGCACUACCUGGACGAGAUCAUCGAGCAGAUCUCCGAGUUCUCCAAGCGG |
| | | GUGAUCCUGGCCGACGCCAACCUGGACAAGGUGCUGUCCGCCUACAACAAGCACCGGGACAAGCCCAUCCGGGAGCAGGCCGAGAACAUCA |
| | | UCCACCUGUUCACCCUGACCAACCUGGGCGCCCCCGCCGCCUUCAAGUACUUCGACACCACCAUCGACAAGAAGCGGUACACCUCCACCAA |
| | | GGAGGUGCUGGACGCCACCCUGAUCCACCAGUCCAUCACCGGCCUGUACGAGACCCGGAUCGACCUGUCCCAGCUGGGCGGCGACGGCGGC |
| | | GGCUCCCCCAAGAAGAAGCGGAAGGUGGAGUAGCUAGCACCAGCCUCAAGAACACCCGAAUGGAGUCUCUAAGCUACAUAAUACCAACUUA |
| | | CACUUUACAAAAUGUUGUCCCCCAAAAUGUAGCCAUUCGUAUCUGCUCCUAAUAAAAAGAAAGUUUCUUCACAUUCUCUCGAGAAAAAAAA |
| | | AAAAUGGAAAAAAAAAAAACGGAAAAAAAAAAAAGGUAAAAAAAAAAAAUAUAAAAAAAAAAAACAUAAAAAAAAAAAACGAAAAAAAAAA |
| | | AACGUAAAAAAAAAAAACUCAAAAAAAAAAAGAUAAAAAAAAAAAACCUAAAAAAAAAAAAUGUAAAAAAAAAAAAGGGAAAAAAAAAAAA |
| | | CGCAAAAAAAAAAAACACAAAAAAAAAAAAUGCAAAAAAAAAAAAUCGAAAAAAAAAAAAUCUAAAAAAAAAAAACGAAAAAAAAAAAACC |
| | | CAAAAAAAAAAAAGACAAAAAAAAAAAAUAGAAAAAAAAAAAGUUAAAAAAAAAAAACUGAAAAAAAAAAAAUUUAAAAAAAAAAAAUCUA |
| | | G |
| 27 | Spy- | augAUCAAGAUCGCCACCCGGAAGUACCUGGGCAAGCAGAACGUGUACGACAUCGGCGUGGAGCGGgaccacaacUUCGCCCUGAAGAACG |
| | R1333K | GCUUCAUCGCCUCCAACUGCUGGGAGGGCUCCGGCgacaagaaguacuccaucggccuggacaucggcaccaacuccgugggcugggccgu |
| | Cas9 | gaucaccgacgaguacaaggugcccuccaagaaguucaaggugcugggcaacaccgaccggcacuccaucaagaagaaccugaucggcgcc |
| | Intein | cugcuguucgacuccggcgagaccgccgaggccacccggcugaagcggaccgcccggcggcgguacacccggcggaagaaccggaucugcu |
| | (mRNA H)- | accugcaggagaucuucuccaacgagauggccaagguggacgacuccuucuuccaccggcuggaggaguccuuccugguggaggaggacaa |
| | ORF | gaagcacgagcggcaccccaucuucggcaacaucguggacgagguggccuaccacgagaaguaccccaccaucuaccaccugcggaagaag |
| | sequence | cugguggacuccaccgacaaggccgaccugcggcugaucuaccuggcccuggcccacaugaucaaguuccggggccacuuccugaucgagg |
| | | gcgaccugaaccccgacaacuccgacguggacaagcuguucauccagcuggugcagaccuacaaccagcuguucgaggagaaccccaucaa |
| | | cgccuccggcguggacgccaaggccauccuguccgcccggcuguccaagucccggcggcuggagaaccugaucgcccagcugcccggcgag |
| | | aagaagaacggccuguucggcaaccugaucgcccugucccugggccugacccccaacuucaaguccaacuucgaccuggccgaggacgcca |
| | | agcugcagcuguccaaggacaccuacgacgacgaccuggacaaccugcuggcccagaucggcgaccaguacgccgaccuguuccuggccgc |
| | | caagaaccuguccgacgccauccugcuguccgacauccugcgggugaacaccgagaucaccaaggccccccuguccgccuccaugaucaag |
| | | cgguacgacgagcaccaccaggaccugacccugcugaaggcccuggugcggcagcagcugcccgagaaguacaaggagaucuucuucgacc |
| | | aguccaagaacggcuacgccggcuacaucgacggcggcgccucccaggaggaguucuacaaguucaucaagcccauccuggagaagaugga |
| | | cggcaccgaggagcugcuggugaagcugaaccgggaggaccugcugcggaagcagcggaccuucgacaacggcuccaucccccaccagauc |
| | | caccugggcgagcugcacgccauccugcggcggcaggaggacuucuaccccuuccugaaggacaaccgggagaagaucgagaagauccuga |
| | | ccuuccggauccccuacuacgugggcccccuggcccggggcaacucccgguucgccuggaugacccggaaguccgaggagaccaucacccc |
| | | cuggaacuucgaggaggugguggacaagggcgccuccgcccaguccuucaucgagcggAUGACCAACUUCGACAAGAACCUGCCCAACGAG |
| | | AAGGUGCUGCCCAAGCACUCCCUGCUGUACGAGUACUUCACCGUGUACAACGAGCUGACCAAGGUGAAGUACGUGACCGAGGGCAUGCGGA |
| | | AGCCCGCCUUCCUGUCCGGCGAGCAGAAGAAGGCCAUCGUGGACCUGCUGUUCAAGACCAACCGGAAGGUGACCGUGAAGCAGCUGAAGGA |
| | | GGACUACUUCAAGAAGAUCGAGUGCUUCGACUCCGUGGAGAUCUCCGGCGUGGAGGACCGGUUCAACGCCUCCCUGGGCACCUACCACGAC |
| | | CUGCUGAAGAUCAUCAAGGACAAGGACUUCCUGGACAACGAGGAGAACGAGGACAUCCUGGAGGACAUCGUGCUGACCCUGACCCUGUUCG |
| | | AGGACCGGGAGAUGAUCGAGGAGCGGCUGAAGACCUACGCCCACCUGUUCGACGACAAGGUGAUGAAGCAGCUGAAGCGGCGGCGGUACAC |
| | | CGGCUGGGGCCGgcugucccggaagcugaucaacggcauccgggacaagcaguccggcaagaccauccuggacuuccugaaguccgacggc |
| | | uucgccaaccggaacuucaugcagcugauccacgacgacucccugaccuucaaggaggacauccagaaggcccagguguccggccagggcg |
| | | acucccugcacgagcacaucgccaaccuggccggcucccccgccaucaagaagggcauccugcagaccgugaaggugguggacgagcuggu |
| | | gaaggugaugggccggcacaagcccgagaacaucgugaucgagauggcccgggagaaccagaccacccagaagggccagaagaacucccgg |
| | | gagcggaugaagcggaucgaggagggcaucaaggagcugggcucccagauccugaaggagcaccccguggagaacacccagcugcagaacg |
| | | agaagcuguaccuguacuaccugcagaacggccgggacauguacguggaccaggagcuggacaucaaccggcuguccgacuacgacgugga |
| | | ccacaucgugccccaguccuuccugaaggacgacuccaucgacaacaaggugcugacccgguccgacaagaaccggggcaaguccgacaac |
| | | gugcccuccgaggagguggugaagaagaugaagaacuacuggcggcagcugcugaacgccaagcugaucacccagcggaaguucgacaacc |
| | | ugaccaaggccgagcggggcggccuguccgagcuggacaaggccggcuucaucaagcggcagcugguggagacccggcagaucaccaagca |
| | | cguggcccagauccuggacucccggaugaacaccaaguacgacgagaacgacaagcugauccgggaggugaaggugaucacccugaagucc |
| | | aagcugguguccgacuuccggaaggacuuccaguucuacaagguggcggagaucaacaacuaccaccacgcccacgacgccuaccugaacg |
| | | ccguggugggcaccgcccugaucaagaaguaccccaagcuggaguccgaguucguguacggcgacuacaagguguacgacgugcggaagau |
| | | gaucgccaaguccgagcaggagaucggcaaggccaccgccaaguacuucuucuacuccaacaucaugaacuucuucaagaccgagaucacc |
| | | cuggccaacggcgagauccggaagcggccccugaucgagaccaacggcgagaccggcgagaucgugugggacaagggccgggacuucgcca |
| | | ccgugcggaaggugcuguccaugccccaggugaacaucgugaagaagaccgaggugcagaccgcgggcuucuccaaggaguccauccugcc |
| | | caagcggaacuccgacaagcugaucgcccggaagaaggacugggaccccaagaaguacggcggcuucgacucccccaccguggccuacucc |
| | | gugcuggugguggccaagguggagaagggcaaguccaagaagcugaaguccgugaaggagcugcugggcaucaccaucauggagcgguccu |
| | | ccuucgagaagaaccccaucgacuuccuggaggccaagggcuacaaggaggugaagaaggaccugaucaucaagcugcccaaguacucccu |
| | | guucgagcuggagaacggccggaagcggaugcuggccuccgccggcgagcugcagaagggcaacgagcuggcccugcccuccaaguacgug |
| | | aacuuccuguaccuggccucccacuacgagaagcugaagggcucccccgaggacaacgagcagaagcagcuguucguggagcagcacaagc |
| | | acuaccuggacgagaucaucgagcagaucuccgaguucuccaagcgggugauccuggccgacgccaaccuggacaaggugcuguccgccua |
| | | caacaagcaccgggacaagcccauccgggagcaggccgagaacaucauccaccuguucacccugaccaaccugcgggcccccgccgccuuc |
| | | aaguacuucgacaccaccaucgacAAGaagcgguacaccuccaccaaggaggugcuggacgccacccugauccaccaguccaucaccggcc |
| | | uguacgagacccggaucgaccugucccagcugggggcgacGGCGGCGGCUCCCCCAAGAAGAAGCGGAAGGUGGAGuag |
| 28 | Spy- | MIKIATRKYLGKQNVYDIGVERDHNFALKNGFIASNCWEGSGDKKYSIGLDIGTNSVGWAVITDEYKVPSKKFKVLGNTDRHSIKKNLIGA |
| | R1333K | LLFDSGETAEATRLKRTARRRYTRRKNRICYLQEIFSNEMAKVDDSFFHRLEESFLVEEDKKHERHPIFGNIVDEVAYHEKYPTIYHLRKK |
| | Cas9 | LVDSTDKADLRLIYLALAHMIKFRGHFLIEGDLNPDNSDVDKLFIQLVQTYNQLFEENPINASGVDAKAILSARLSKSRRLENLIAQLPGE |
| | Intein | KKNGLFGNLIALSLGLTPNFKSNFDLAEDAKLQLSKDTYDDDLDNLLAQIGDQYADLFLAAKNLSDAILLSDILRVNTEITKAPLSASMIK |
| | (mRNA H)- | RYDEHHQDLTLLKALVRQQLPEKYKEIFFDQSKNGYAGYIDGGASQEEFYKFIKPILEKMDGTEELLVKLNREDLLRKQRTFDNGSIPHQI |
| | amino | HLGELHAILRRQEDFYPFLKDNREKIEKILTFRIPYYVGPLARGNSRFAWMTRKSEETITPWNFEEVVDKGASAQSFIERMTNFDKNLPNE |
| | acid | KVLPKHSLLYEYFTVYNELTKVKYVTEGMRKPAFLSGEQKKAIVDLLFKTNRKVTVKQLKEDYFKKIECFDSVEISGVEDRFNASLGTYHD |
| | sequence | LLKIIKDKDFLDNEENEDILEDIVLTLTLFEDREMIEERLKTYAHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKKGILQTVKVVDELVKVMGRHKPENIVIEMARENQTTQKGQKNSR |
| | | ERMKRIEEGIKELGSQILKEHPVENTQLQNEKLYLYYLQNGRDMYVDQELDINRLSDYDVDHIVPQSFLKDDSIDNKVLTRSDKNRGKSDN |
| | | VPSEEVVKKMKNYWRQLLNAKLITQRKFDNLTKAERGGLSELDKAGFIKRQLVETRQITKHVAQILDSRMNTKYDENDKLIREVKVITLKS |
| | | KLVSDFRKDFQFYKVREINNYHHAHDAYLNAVVGTALIKKYPKLESEFVYGDYKVYDVRKMIAKSEQEIGKATAKYFFYSNIMNFFKTEIT |
| | | LANGEIRKRPLIETNGETGEIVWDKGRDFATVRKVLSMPQVNIVKKTEVQTGGFSKESILPKRNSDKLIARKKDWDPKKYGGFDSPTVAYS |
| | | VLVVAKVEKGKSKKLKSVKELLGITIMERSSFEKNPIDFLEAKGYKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVILADANLDKVLSAYNKHRDKPIREQAENIIHLFTLTNLGAPAAF |
| | | KYFDTTIDKKRYTSTKEVLDATLIHQSITGLYETRIDLSQLGGDGGGSPKKKRKVE* |
| 29 | Spy- | GGGAAGCUCAGAAUAAACGCUCAACUUUGGCCGGAUCUGCCACCAUGGACGGCUCCGGGGGGGGCUCCCCCAAGAAGAAGCGGAAGGUGGG |
| | R1333K | CUCCGGCGACAAGAAGUACUCCAUCGGCCUGGACAUCGGCACCAACUCCGUGGGCUGGGCCGUGAUCACCGACGAGUACAAGGUGCCCUCC |
| | Cas9 | AAGAAGUUCAAGGUGCUGGGCAACACCGACCGGCACUCCAUCAAGAAGAACCUGAUCGGCGCCCUGCUGUUCGACUCCGGCGAGACCGCCG |
| | Intein | AGGCCACCCGGCUGAAGCGGACCGCCCGGCGGCGGUACACCCGGCGGAAGAACCGGAUCUGCUACCUGCAGGAGAUCUUCUCCAACGAGAU |
| | (mRNA I)- | GGCCAAGGUGGACGACUCCUUCUUCCACCGGCUGGAGGAGUCCUUCCUGGUGGAGGAGGACAAGAAGCACGAGCGGCACCCCAUCUUCGGC |
| | mRNA | AACAUCGUGGACGAGGUGGCCUACCACGAGAAGUACCCCACCAUCUACCACCUGCGGAAGAAGCUGGUGGACUCCACCGACAAGGCCGACC |
| | sequence | UGCGGCUGAUCUACCUGGCCCUGGCCCACAUGAUCAAGUUCCGGGGCCACUUCCUGAUCGAGGGCGACCUGAACCCCGACAACUCCGACGU |
| | | GGACAAGCUGUUCAUCCAGCUGGUGCAGACCUACAACCAGCUGUUCGAGGAGAACCCCAUCAACGCCUCCGGCGUGGACGCCAAGGCCAUC |
| | | CUGUCCGCCCGGCUGUCCAAGUCCCGGCGGCUGGAGAACCUGAUCGCCCAGCUGCCCGGCGAGAAGAAGAACGGCCUGUUCGGCAACCUGA |
| | | UCGCCCUGUCCCUGGGCCUGACCCCCAACUUCAAGUCCAACUUCGACCUGGCCGAGGACGCCAAGCUGCAGCUGUCCAAGGACACCUACGA |
| | | CGACGACCUGGACAACCUGCUGGCCCAGAUCGGCGACCAGUACGCCGACCUGUUCCUGGCCGCCAAGAACCUGUCCGACGCCAUCCUGCUG |
| | | UCCGACAUCCUGCGGGUGAACACCGAGAUCACCAAGGCCCCCCUGUCCGCCUCCAUGAUCAAGCGGUACGACGAGCACCACCAGGACCUGA |
| | | CCCUGCUGAAGGCCCUGGUGCGGCAGCAGCUGCCCGAGAAGUACAAGGAGAUCUUCUUCGACCAGUCCAAGAACGGCUACGCCGGCUACAU |
| | | CGACGGCGGCGCCUCCCAGGAGGAGUUCUACAAGUUCAUCAAGCCCAUCCUGGAGAAGAUGGACGGCACCGAGGAGCUGCUGGUGAAGCUG |
| | | AACCGGGAGGACCUGCUGCGGAAGCAGCGGACCUUCGACAACGGCUCCAUCCCCCACCAGAUCCACCUGGGCGAGCUGCACGCCAUCCUGC |
| | | GGCGGCAGGAGGACUUCUACCCCUUCCUGAAGGACAACCGGGAGAAGAUCGAGAAGAUCCUGACCUUCCGGAUCCCCUACUACGUGGGCCC |
| | | CCUGGCCCGGGGCAACUCCCGGUUCGCCUGGAUGACCCGGAAGUCCGAGGAGACCAUCACCCCCUGGAACUUCGAGGAGGUGGUGGACAAG |
| | | GGCGCCUCCGCCCAGUCCUUCAUCGAGCGGAUGACCAACUUCGACAAGAACCUGCCCAACGAGAAGGUGCUGCCCAAGCACUCCCUGCUGU |
| | | ACGAGUACUUCACCGUGUACAACGAGCUGACCAAGGUGAAGUACGUGACCGAGGGCAUGCGGAAGCCCGCCUUCCUGUCCGGCGAGCAGAA |
| | | GAAGGCCAUCGUGGACCUGCUGUUCAAGACCAACCGGAAGGUGACCGUGAAGCAGCUGAAGGAGGACUACUUCAAGAAGAUCGAGUGCUUC |
| | | GACUCCGUGGAGAUCUCCGGCGUGGAGGACCGGUUCAACGCCUCCCUGGGCACCUACCACGACCUGCUGAAGAUCAUCAAGGACAAGGACU |
| | | UCCUGGACAACGAGGAGAACGAGGACAUCCUGGAGGACAUCGUGCUGACCCUGACCCUGUUCGAGGACCGGGAGAUGAUCGAGGAGCGGCU |
| | | GAAGACCUACGCCCACCUGUUCGACGACAAGGUGAUGAAGCAGCUGAAGCGGCGGCGGUACACCGGCUGGGGCCGGCUGUCCCGGAAGCUG |
| | | AUCAACGGCAUCCGGGACAAGCAGUCCGGCAAGACCAUCCUGGACUUCCUGAAGUCCGACGGCUUCGCCAACCGGAACUUCAUGCAGCUGA |
| | | UCCACGACGACUCCCUGACCUUCAAGGAGGACAUCCAGAAGGCCCAGGUGUCCGGCCAGGGCGACUCCCUGCACGAGCACAUCGCCAACCU |
| | | GGCCGGCUCCCCCGCCAUCAAGAAGGGCAUCCUGCAGACCGUGAAGGUGGUGGACGAGCUGGUGAAGGUGAUGGGCCGGCACAAGCCCGAG |
| | | AACAUCGUGAUCGAGAUGGCCCGGGAGAACCAGACCACCCAGAAGGGCCAGAAGAACUCCCGGGAGCGGAUGAAGCGGAUCGAGGAGGGCA |
| | | UCAAGGAGCUGGGCUCCCAGAUCCUGAAGGAGCACCCCGUGGAGAACACCCAGCUGCAGAACGAGAAGCUGUACCUGUACUACCUGCAGAA |
| | | CGGCCGGGACAUGUACGUGGACCAGGAGCUGGACAUCAACCGGCUGUCCGACUACGACGUGGACCACAUCGUGCCCCAGUCCUUCCUGAAG |
| | | GACGACUCCAUCGACAACAAGGUGCUGACCCGGUCCGACAAGAACCGGGGCAAGUCCGACAACGUGCCCUCCGAGGAGGUGGUGAAGAAGA |
| | | UGAAGAACUACUGGCGGCAGCUGCUGAACGCCAAGCUGAUCACCCAGCGGAAGUUCGACAACCUGACCAAGGCCGAGCGGGGCGGCCUGUC |
| | | CGAGCUGGACAAGGCCGGCUUCAUCAAGCGGCAGCUGGUGGAGACCCGGCAGAUCACCAAGCACGUGGCCCAGAUCCUGGACUCCCGGAUG |
| | | AACACCAAGUACGACGAGAACGACAAGCUGAUCCGGGAGGUGAAGGUGAUCACCCUGAAGUCCAAGCUGGUGUCCGACUUCCGGAAGGACU |
| | | UCCAGUUCUACAAGGUGCGGGAGAUCAACAACUACCACCACGCCCACGACGCCUACCUGAACGCCGUGGUGGGCACCGCCCUGAUCAAGAA |
| | | GUACCCCAAGCUGGAGUCCGAGUUCGUGUACGGCGACUACAAGGUGUACGACGUGCGGAAGAUGAUCGCCAAGUCCGAGCAGGAGAUCGGC |
| | | AAGGCCACCGCCAAGUACUUCUUCUACUCCAACAUCAUGAACUUCUUCAAGACCGAGAUCACCCUGGCCAACGGCGAGAUCCGGAAGCGGC |
| | | CCCUGAUCGAGACCAACGGCGAGACCGGCGAGAUCGUGUGGGACAAGGGCCGGGACUUCGCCACCGUGCGGAAGGUGCUGUCCAUGCCCCA |
| | | GGUGAACAUCGUGAAGAAGACCGAGGUGCAGACCGGCGGCUUCUCCAAGGAGUCCAUCCUGCCCAAGCGGAACUCCGACAAGCUGAUCGCC |
| | | CGGAAGAAGGACUGGGACCCCAAGAAGUACGGCGGCUUCGACUCCCCCACCGUGGCCUACUCCGUGCUGGUGGUGGCCAAGGUGGAGAAGG |
| | | GCAAGUCCAAGAAGCUGAAGUCCGUGAAGGAGCUGCUGGGCAUCACCAUCAUGGAGCGGUCCUCCUUCGAGAAGAACCCCAUCGACUUCCU |
| | | GGAGGCCAAGGGCUACAAGGAGGUGAAGAAGGACCUGAUCAUCAAGCUGCCCAAGUACUCCCUGUUCGAGCUGGAGAACGGCCGGAAGCGG |
| | | AUGCUGGCCUCCGCCGGCGAGCUGCAGAAGGGCAACGAGCUGGCCCUGCCCUCCAAGUACGUGAACUUCCUGUACCUGGCCUCCCACUACG |
| | | AGAAGCUGAAGGGCUCCCCCGAGGACAACGAGCAGAAGCAGCUGUUCGUGGAGCAGCACAAGCACUACCUGGACGAGAUCAUCGAGCAGAU |
| | | CUCCGAGUUCUCCAAGCGGGUGAUCCUGGCCGACGCCAACCUGGACAAGGUGCUGUCCGCCUACAACAAGCACCGGGACAAGCCCAUCCGG |
| | | GAGCAGGCCGAGAACAUCAUCCACCUGUUCACCCUGACCAACCUGGGCGCCCCCGCCGCCUUCAAGUACUUCGACACCACCAUCGACAAGA |
| | | AGCGGUACACCUCCACCAAGGAGGUGCUGGACGCCACCCUGAUCCACCAGUCCAUCACCGGCCUGUACGAGACCCGGAUCGACCUGUCCCA |
| | | GCUGGGCGGCGACCGGGGCAAGUGCCUGUCCUACGAGACCGAGAUCCUGACCGUGGAGUACGGCCUGCUGCCCAUCGUGGUGGUGGUGGAG |
| | | AAGCGGAUCGAGUGCACCGUGUACUCCGUGGACAACAACGGCAACAUCUACACCCAGCCCGUGGCCCAGUGGCACGACCGGGGCGAGCAGG |
| | | AGGUGUUCGAGUACUGCCUGGAGGACGGCUCCCUGAUCCGGGCCACCAAGGACCACAAGUUCAUGACCGUGGACGGCCAGAUGCUGCCCAU |
| | | CGACGAGAUCUUCGAGCGGGAGCUGGACCUGAUGCGGGUGGACAACCUGCCCAACUAGCUAGCACCAGCCUCAAGAACACCCGAAUGGAGU |
| | | CUCUAAGCUACAUAAUACCAACUUACACUUUACAAAAUGUUGUCCCCCAAAAUGUAGCCAUUCGUAUCUGCUCCUAAUAAAAAGAAAGUUU |
| | | CUUCACAUUCUCUCGAGAAAAAAAAAAAAUGGAAAAAAAAAAAACGGAAAAAAAAAAAAGGUAAAAAAAAAAAAUAUAAAAAAAAAAAACA |
| | | UAAAAAAAAAAAACGAAAAAAAAAAAACGUAAAAAAAAAAAACUCAAAAAAAAAAAGAUAAAAAAAAAAAACCUAAAAAAAAAAAAUGUAA |
| | | AAAAAAAAAAGGGAAAAAAAAAAAACGCAAAAAAAAAAAACACAAAAAAAAAAAAUGCAAAAAAAAAAAAUCGAAAAAAAAAAAAUCUAAA |
| | | AAAAAAAAACGAAAAAAAAAAAACCCAAAAAAAAAAAAGACAAAAAAAAAAAAUAGAAAAAAAAAAAGUUAAAAAAAAAAAACUGAAAAAA |
| | | AAAAAAUUUAAAAAAAAAAAAUCUAG |
| 30 | Spy- | augGACGGCUCCGGCGGCGGCUCCCCCAAGAAGAAGCGGAAGGUGGGCUCCGGCgacaagaaguacuccaucggccuggacaucggcacca |
| | R1333K | acuccgugggcugggccgugaucaccgacgaguacaaggugcccuccaagaaguucaaggugcugggcaacaccgaccggcacuccaucaa |
| | Cas9 | gaagaaccugaucggcgcccugcuguucgacuccggcgagaccgccgaggccacccggcugaagcggaccgcccggcggcgguacacccgg |
| | Intein | cggaagaaccggaucugcuaccugcaggagaucuucuccaacgagauggccaagguggacgacuccuucuuccaccggcuggaggaguccu |
| | (mRNA I)- | uccugguggaggaggacaagaagcacgagcggcaccccaucuucggcaacaucguggacgagguggccuaccacgagaaguaccccaccau |
| | ORF | cuaccaccugcggaagaagcugguggacuccaccgacaaggccgaccugcggcugaucuaccuggcccuggcccacaugaucaaguuccgg |
| | sequence | ggccacuuccugaucgagggcgaccugaaccccgacaacuccgacguggacaagcuguucauccagcuggugcagaccuacaaccagcugu |
| | | ucgaggagaaccccaucaacgccuccggcguggacgccaaggccauccuguccgcccggcuguccaagucccggcggcuggagaaccugau |
| | | cgcccagcugcccggcgagaagaagaacggccuguucggcaaccugaucgcccugucccugggccugacccccaacuucaaguccaacuuc |
| | | gaccuggccgaggacgccaagcugcagcuguccaaggacaccuacgacgacgaccuggacaaccugcuggcccagaucggcgaccaguacg |
| | | ccgaccuguuccuggccgccaagaaccuguccgacgccauccugcuguccgacauccugcgggugaacaccgagaucaccaaggccccccu |
| | | guccgccuccaugaucaagcgguacgacgagcaccaccaggaccugacccugcugaaggcccuggugcggcagcagcugcccgagaaguac |
| | | aaggagaucuucuucgaccaguccaagaacggcuacgccggcuacaucgacgggcgcgccucccaggaggaguucuacaaguucaucaagc |
| | | ccauccuggagaagauggacggcaccgaggagcugcuggugaagcugaaccgggaggaccugcugcggaagcagcggaccuucgacaacgg |
| | | cuccaucccccaccagauccaccugggcgagcugcacgccauccugcggcggcaggaggacuucuaccccuuccugaaggacaaccgggag |
| | | aagaucgagaagauccugaccuuccggauccccuacuacgugggcccccuggcccggggcaacucccgguucgccuggaugacccggaagu |
| | | ccgaggagaccaucacccccuggaacuucgaggaggugguggacaagggcgccuccgcccaguccuucaucgagcggAUGACCAACUUCGA |
| | | CAAGAACCUGCCCAACGAGAAGGUGCUGCCCAAGCACUCCCUGCUGUACGAGUACUUCACCGUGUACAACGAGCUGACCAAGGUGAAGUAC |
| | | GUGACCGAGGGCAUGCGGAAGCCCGCCUUCCUGUCCGGCGAGCAGAAGAAGGCCAUCGUGGACCUGCUGUUCAAGACCAACCGGAAGGUGA |
| | | CCGUGAAGCAGCUGAAGGAGGACUACUUCAAGAAGAUCGAGUGCUUCGACUCCGUGGAGAUCUCCGGCGUGGAGGACCGGUUCAACGCCUC |
| | | CCUGGGCACCUACCACGACCUGCUGAAGAUCAUCAAGGACAAGGACUUCCUGGACAACGAGGAGAACGAGGACAUCCUGGAGGACAUCGUG |
| | | CUGACCCUGACCCUGUUCGAGGACCGGGAGAUGAUCGAGGAGCGGCUGAAGACCUACGCCCACCUGUUCGACGACAAGGUGAUGAAGCAGC |
| | | UGAAGCGGCGGCGGUACACCGGCUGGGGCCGgcugucccggaagcugaucaacggcauccgggacaagcaguccggcaagaccauccugga |
| | | cuuccugaaguccgacggcuucgccaaccggaacuucaugcagcugauccacgacgacucccugaccuucaaggaggacauccagaaggcc |
| | | cagguguccggccagggcgacucccugcacgagcacaucgccaaccuggccggcucccccgccaucaagaagggcauccugcagaccguga |
| | | aggugguggacgagcuggugaaggugaugggccggcacaagcccgagaacaucgugaucgagauggcccgggagaaccagaccacccagaa |
| | | gggccagaagaacucccgggagcggaugaagcggaucgaggagggcaucaaggagcugggcucccagauccugaaggagcaccccguggag |
| | | aacacccagcugcagaacgagaagcuguaccuguacuaccugcagaacggccgggacauguacguggaccaggagcuggacaucaaccggc |
| | | uguccgacuacgacguggaccacaucgugccccaguccuuccugaaggacgacuccaucgacaacaaggugcugacccgguccgacaagaa |
| | | ccggggcaaguccgacaacgugcccuccgaggagguggugaagaagaugaagaacuacuggcggcagcugcugaacgccaagcugaucacc |
| | | cagcggaaguucgacaaccugaccaaggccgagcgcgggggccuguccgagcuggacaaggccggcuucaucaagcggcagcugguggaga |
| | | cccggcagaucaccaagcacguggcccagauccuggacucccggaugaacaccaaguacgacgagaacgacaagcugauccgggaggugaa |
| | | ggugaucacccugaaguccaagcugguguccgacuuccggaaggacuuccaguucuacaagguggcggagaucaacaacuaccaccacgcc |
| | | cacgacgccuaccugaacgccguggugggcaccgcccugaucaagaaguaccccaagcuggaguccgaguucguguacggcgacuacaagg |
| | | uguacgacgugcggaagaugaucgccaaguccgagcaggagaucggcaaggccaccgccaaguacuucuucuacuccaacaucaugaacuu |
| | | cuucaagaccgagaucacccuggccaacggcgagauccggaagcggccccugaucgagaccaacggcgagaccggcgagaucgugugggac |
| | | aagggccgggacuucgccaccgugcggaaggugcuguccaugccccaggugaacaucgugaagaagaccgagcgugcagaccggggcuucu |
| | | ccaaggaguccauccugcccaagcggaacuccgacaagcugaucgcccggaagaaggacugggaccccaagaaguacggcggcuucgacuc |
| | | ccccaccguggccuacuccgugcuggugguggccaagguggagaagggcaaguccaagaagcugaaguccgugaaggagcugcugggcauc |
| | | accaucauggagcgguccuccuucgagaagaaccccaucgacuuccuggaggccaagggcuacaaggaggugaagaaggaccugaucauca |
| | | agcugcccaaguacucccuguucgagcuggagaacggccggaagcggaugcuggccuccgccggcgagcugcagaagggcaacgagcuggc |
| | | ccugcccuccaaguacgugaacuuccuguaccuggccucccacuacgagaagcugaagggcucccccgaggacaacgagcagaagcagcug |
| | | uucguggagcagcacaagcacuaccuggacgagaucaucgagcagaucuccgaguucuccaagcgggugauccuggccgacgccaaccugg |
| | | acaaggugcuguccgccuacaacaagcaccgggacaagcccauccgggagcaggccgagaacaucauccaccuguucacccugaccaaccu |
| | | gggcgcccccgccgccuucaaguacuucgacaccaccaucgacAAGaagcgguacaccuccaccaaggaggugcuggacgccacccugauc |
| | | caccaguccaucaccggccuguacgagacccggaucgaccugucccagcuggcgggcgacCGGGGCAAGUGCCUGUCCUACGAGACCGAGA |
| | | UCCUGACCGUGGAGUACGGCCUGCUGCCCAUCGUGGUGGUGGUGGAGAAGCGGAUCGAGUGCACCGUGUACUCCGUGGACAACAACGGCAA |
| | | CAUCUACACCCAGCCCGUGGCCCAGUGGCACGACCGGGGCGAGCAGGAGGUGUUCGAGUACUGCCUGGAGGACGGCUCCCUGAUCCGGGCC |
| | | ACCAAGGACCACAAGUUCAUGACCGUGGACGGCCAGAUGCUGCCCAUCGACGAGAUCUUCGAGCGGGAGCUGGACCUGAUGCGGGUGGACA |
| | | ACCUGCCCAACuag |
| 31 | Spy- | MDGSGGGSPKKKRKVGSGDKKYSIGLDIGTNSVGWAVITDEYKVPSKKFKVLGNTDRHSIKKNLIGALLFDSGETAEATRLKRTARRRYTR |
| | R1333K | RKNRICYLQEIFSNEMAKVDDSFFHRLEESFLVEEDKKHERHPIFGNIVDEVAYHEKYPTIYHLRKKLVDSTDKADLRLIYLALAHMIKFR |
| | Cas9 | GHFLIEGDLNPDNSDVDKLFIQLVQTYNQLFEENPINASGVDAKAILSARLSKSRRLENLIAQLPGEKKNGLFGNLIALSLGLTPNFKSNF |
| | Intein | DLAEDAKLQLSKDTYDDDLDNLLAQIGDQYADLFLAAKNLSDAILLSDILRVNTEITKAPLSASMIKRYDEHHQDLTLLKALVRQQLPEKY |
| | (mRNA I)- | KEIFFDQSKNGYAGYIDGGASQEEFYKFIKPILEKMDGTEELLVKLNREDLLRKQRTFDNGSIPHQIHLGELHAILRRQEDFYPFLKDNRE |
| | amino | KIEKILTFRIPYYVGPLARGNSRFAWMTRKSEETITPWNFEEVVDKGASAQSFIERMTNFDKNLPNEKVLPKHSLLYEYFTVYNELTKVKY |
| | acid | VTEGMRKPAFLSGEQKKAIVDLLFKTNRKVTVKQLKEDYFKKIECFDSVEISGVEDRFNASLGTYHDLLKIIKDKDFLDNEENEDILEDIV |
| | sequence | LTLTLFEDREMIEERLKTYAHLFDDKVMKQLKRRRYTGWGRLSRKLINGIRDKQSGKTILDFLKSDGFANRNFMQLIHDDSLTFKEDIQKA |
| | | QVSGQGDSLHEHIANLAGSPAIKKGILQTVKVVDELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVITLKSKLVSDFRKDFQFYKVREINNYHHA |
| | | HDAYLNAVVGTALIKKYPKLESEFVYGDYKVYDVRKMIAKSEQEIGKATAKYFFYSNIMNFFKTEITLANGEIRKRPLIETNGETGEIVWD |
| | | KGRDFATVRKVLSMPQVNIVKKTEVQTGGFSKESILPKRNSDKLIARKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRMLASAGELQKGNELALPSKYVNFLYLASHYEKLKGSPEDNEQKQL |
| | | FVEQHKHYLDEIIEQISEFSKRVILADANLDKVLSAYNKHRDKPIREQAENIIHLFTLTNLGAPAAFKYFDTTIDKKRYTSTKEVLDATLI |
| | | HQSITGLYETRIDLSQLGGDRGKCLSYETEILTVEYGLLPIVVVVEKRIECTVYSVDNNGNIYTQPVAQWHDRGEQEVFEYCLEDGSLIRA |
| | | TKDHKFMTVDGQMLPIDEIFERELDLMRVDNLPN* |
| 32 | Nme2Cas9 | GGGAAGCUCAGAAUAAACGCUCAACUUUGGCCGGAUCUGCCACCAUGAUCAAGAUCGCCACCCGGAAGUACCUGGGCAAGCAGAACGUGUA |
| | Intein | CGACAUCGGCGUGGAGCGGGACCACAACUUCGCCCUGAAGAACGGCUUCAUCGCCUCCAACUGCUGGGAGGGCGCCCCCGAGUCCGCCACC |
| | (mRNA J)- | GAGUCCGGCGGCACCUCCACCGAGUCCGAGGGCUCCGCCGGCACCUCCACCGAGUCCGAGGGCUCCGCCGGCUCCGCCGGCUCCACCUCCG |
| | mRNA | GCGGCUCCGGCGGCGGCUCCGGCGGCUCCGGCUCCGCCGGCACCUCCACCGAGUCCGAGGGCUCCGCCGGCACCUCCGAGUCCGCCACCGA |
| | sequence | GUCCGGCGGCACCUCCACCGAGUCCGAGGGCUCCUCCUCCGGCGCCGCCUUCAAGCCCAACCCCAUCAACUACAUCCUGGGCCUGGACAUC |
| | | GGCAUCGCCUCCGUGGGCUGGGCCAUGGUGGAGAUCGACGAGGAGGAGAACCCCAUCCGGCUGAUCGACCUGGGCGUGCGGGUGUUCGAGC |
| | | GGGCCGAGGUGCCCAAGACCGGCGACUCCCUGGCCAUGGCCCGGCGGCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCAGGCCGCCGACUUCGACGAGAACGGCCUGAUCAAGUCCCUGCCCAAC |
| | | ACCCCCUGGCAGCUGCGGGCCGCCGCCCUGGACCGGAAGCUGACCCCCCUGGAGUGGUCCGCCGUGCUGCUGCACCUGAUCAAGCACCGGG |
| | | GCUACCUGUCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGGGCGACUAC |
| | | UCCCACACCUUCUCCCGGAAGGACCUGCAGGCCGAGCUGAUCCUGCUGUUCGAGAAGCAGAAGGAGUUCGGCAACCCCCACGUGUCCGGCG |
| | | GCCUGAAGGAGGGCAUCGAGACCCUGCUGAUGACCCAGCGGCCCGCCCUGUCCGGCGACGCCGUGCAGAAGAUGCUGGGCCACUGCACCUU |
| | | CGAGCCCGCCGAGCCCAAGGCCGCCAAGAACACCUACACCGCCGAGCGGUUCAUCUGGCUGACCAAGCUGAACAACCUGCGGAUCCUGGAG |
| | | CAGGGCUCCGAGCGGCCCCUGACCGACACCGAGCGGGCCACCCUGAUGGACGAGCCCUACCGGAAGUCCAAGCUGACCUACGCCCAGGCCC |
| | | GGAAGCUGCUGGGCCUGGAGGACACCGCCUUCUUCAAGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAGUCCCCCCUGAACCUGUCCUCCGAGCUGCAGGACGAG |
| | | AUCGGCACCGCCUUCUCCCUGUUCAAGACCGACGAGGACAUCACCGGCCGGCUGAAGGACCGGGUGCAGCCCGAGAUCCUGGAGGCCCUGC |
| | | UGAAGCACAUCUCCUUCGACAAGUUCGUGCAGAUCUCCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAGAAGAUCUACCUGCCCCCCAUCCCCGCCGACGAGAUC |
| | | CGGAACCCCGUGGUGCUGCGGGCCCUGUCCCAGGCCCGGAAGGUGAUCAACGGCGUGGUGCGGCGGUACGGCUCCCCCGCCCGGAUCCACA |
| | | UCGAGACCGCCCGGGAGGUGGGCAAGUCCUUCAAGGACCGGAAGGAGAUCGAGAAGCGGCAGGAGGAGAACCGGAAGGACCGGGAGAAGGC |
| | | CGCCGCCAAGUUCCGGGAGUACUUCCCCAACUUCGUGGGCGAGCCCAAGUCCAAGGACAUCCUGAAGCUGCGGCUGUACGAGCAGCAGCAC |
| | | GGCAAGUGCCUGUACUCCGGCAAGGAGAUCAACCUGGUGCGGCUGAACGAGAAGGGCUACGUGGAGAUCGACCACGCCCUGCCCUUCUCCC |
| | | GGACCUGGGACGACUCCUUCAACAACAAGGUGCUGGUGCUGGGCUCCGAGAACCAGAACAAGGGCAACCAGACCCCCUACGAGUACUUCAA |
| | | CGGCAAGGACAACUCCCGGGAGUGGCAGGAGUUCAAGGCCCGGGUGGAGACCUCCCGGUUCCCCCGGUCCAAGAAGCAGCGGAUCCUGCUG |
| | | CAGAAGUUCGACGAGGACGGCUUCAAGGAGUGCAACCUGAACGACACCCGGUACGUGAACCGGUUCCUGUGCCAGUUCGUGGCCGACCACA |
| | | UCCUGCUGACCGGCAAGGGCAAGCGGCGGGUGUUCGCCUCCAACGGCCAGAUCACCAACCUGCUGCGGGGCUUCUGGGGCCUGCGGAAGGU |
| | | GCGGGCCGAGAACGACCGGCACCACGCCCUGGACGCCGUGGUGGUGGCCUGCUCCACCGUGGCCAUGCAGCAGAAGAUCACCCGGUUCGUG |
| | | CGGUACAAGGAGAUGAACGCCUUCGACGGCAAGACCAUCGACAAGGAGACCGGCAAGGUGCUGCACCAGAAGACCCACUUCCCCCAGCCCU |
| | | GGGAGUUCUUCGCCCAGGAGGUGAUGAUCCGGGUGUUCGGCAAGCCCGACGGCAAGCCCGAGUUCGAGGAGGCCGACACCCCCGAGAAGCU |
| | | GCGGACCCUGCUGGCCGAGAAGCUGUCCUCCCGGCCCGAGGCCGUGCACGAGUACGUGACCCCCCUGUUCGUGUCCCGGGCCCCCAACCGG |
| | | AAGAUGUCCGGCGCCCACAAGGACACCCUGCGGUCCGCCAAGCGGUUCGUGAAGCACAACGAGAAGAUCUCCGUGAAGCGGGUGUGGCUGA |
| | | CCGAGAUCAAGCUGGCCGACCUGGAGAACAUGGUGAACUACAAGAACGGCCGGGAGAUCGAGCUGUACGAGGCCCUGAAGGCCCGGCUGGA |
| | | GGCCUACGGCGGCAACGCCAAGCAGGCCUUCGACCCCAAGGACAACCCCUUCUACAAGAAGGGCGGCCAGCUGGUGAAGGCCGUGCGGGUG |
| | | GAGAAGACCCAGGAGUCCGGCGUGCUGCUGAACAAGAAGAACGCCUACACCAUCGCCGACAACGGCGACAUGGUGCGGGUGGACGUGUUCU |
| | | GCAAGGUGGACAAGAAGGGCAAGAACCAGUACUUCAUCGUGCCCAUCUACGCCUGGCAGGUGGCCGAGAACAUCCUGCCCGACAUCGACUG |
| | | CAAGGGCUACCGGAUCGACGACUCCUACACCUUCUGCUUCUCCCUGCACAAGUACGACCUGAUCGCCUUCCAGAAGGACGAGAAGUCCAAG |
| | | GUGGAGUUCGCCUACUACAUCAACUGCGACUCCUCCAACGGCCGGUUCUACCUGGCCUGGCACGACAAGGGCUCCAAGGAGCAGCAGUUCC |
| | | GGAUCUCCACCCAGAACCUGGUGCUGAUCCAGAAGUACCAGGUGAACGAGCUGGGCAAGGAGAUCCGGCCCUGCCGGCUGAAGAAGCGGCC |
| | | CCCCGUGCGGUCCGGCAAGCGGACCGCCGACGGCUCCGGCGGCGGCUCCCCCAAGAAGAAGCGGAAGGUGGAGUAGCUAGCACCAGCCUCA |
| | | AGAACACCCGAAUGGAGUCUCUAAGCUACAUAAUACCAACUUACACUUUACAAAAUGUUGUCCCCCAAAAUGUAGCCAUUCGUAUCUGCUC |
| | | CUAAUAAAAAGAAAGUUUCUUCACAUUCUCUCGAGAAAAAAAAAAAAUGGAAAAAAAAAAAACGGAAAAAAAAAAAAGGUAAAAAAAAAAA |
| | | AUAUAAAAAAAAAAAACAUAAAAAAAAAAAACGAAAAAAAAAAAACGUAAAAAAAAAAAACUCAAAAAAAAAAAGAUAAAAAAAAAAAACC |
| | | UAAAAAAAAAAAAUGUAAAAAAAAAAAAGGGAAAAAAAAAAAACGCAAAAAAAAAAAACACAAAAAAAAAAAAUGCAAAAAAAAAAAAUCG |
| | | AAAAAAAAAAAAUCUAAAAAAAAAAAACGAAAAAAAAAAAACCCAAAAAAAAAAAAGACAAAAAAAAAAAAUAGAAAAAAAAAAAGUUAAA |
| | | AAAAAAAAACUGAAAAAAAAAAAAUUUAAAAAAAAAAAAUCUAG |
| 33 | Nme2Cas9 | AUGAUCAAGAUCGCCACCCGGAAGUACCUGGGCAAGCAGAACGUGUACGACAUCGGCGUGGAGCGGgaccacaacUUCGCCCUGAAGAACG |
| | Intein | GCUUCAUCGCCUCCAACUGCUGGGAGGGCGCCCCCGAGUCCGCCACCGAGUCCGGCGGCACCUCCACCGAGUCCGAGGGCUCCGCCGGCAC |
| | (mRNA J)- | CUCCACCGAGUCCGAGGGCUCCGCCGGCUCCGCCGGCUCCACCUCCGGCGGCUCCGGCGGCGGCUCCGGCGGCUCCGGCUCCGCCGGCACC |
| | ORF | UCCACCGAGUCCGAGGGCUCCGCCGGCACCUCCGAGUCCGCCACCGAGUCCGGCGGCACCUCCACCGAGUCCGAGGGCUCCUCCUCCGGCG |
| | sequence | CCGCCUUCAAGCCCAACCCCAUCAACUACAUCCUGGGCCUGGACAUCGGCAUCGCCUCCGUGGGCUGGGCCAUGGUGGAGAUCGACGAGGA |
| | | GGAGAACCCCAUCCGGCUGAUCGACCUGGGCGUGCGGGUGUUCGAGCGGGCCGAGGUGCCCAAGACCGGCGACUCCCUGGCCAUGGCCCGG |
| | | CGGCUGGCCCGGUCCGUGCGGCGGCUGACCCGGCGGCGGGCCCACCGGCUGCUGCGGGCCCGGCGGCUGCUGAAGCGGGAGGGCGUGCUGC |
| | | AGGCCGCCGACUUCGACGAGAACGGCCUGAUCAAGUCCCUGCCCAACACCCCCUGGCAGCUGCGGGCCGCCGCCCUGGACCGGAAGCUGAC |
| | | CCCCCUGGAGUGGUCCGCCGUGCUGCUGCACCUGAUCAAGCACCGGGGCUACCUGUCCCAGCGGAAGAACGAGGGCGAGACCGCCGACAAG |
| | | GAGCUGGGCGCCCUGCUGAAGGGCGUGGCCAACAACGCCCACGCCCUGCAGACCGGCGACUUCCGGACCCCCGCCGAGCUGGCCCUGAACA |
| | | AGUUCGAGAAGGAGUCCGGCCACAUCCGGAACCAGCGGGGCGACUACUCCCACACCUUCUCCCGGAAGGACCUGCAGGCCGAGCUGAUCCU |
| | | GCUGUUCGAGAAGCAGAAGGAGUUCGGCAACCCCCACGUGUCCGGCGGCCUGAAGGAGGGCAUCGAGACCCUGCUGAUGACCCAGCGGCCC |
| | | GCCCUGUCCGGCGACGCCGUGCAGAAGAUGCUGGGCCACUGCACCUUCGAGCCCGCCGAGCCCAAGGCCGCCAAGAACACCUACACCGCCG |
| | | AGCGGUUCAUCUGGCUGACCAAGCUGAACAACCUGCGGAUCCUGGAGCAGGGCUCCGAGCGGCCCCUGACCGACACCGAGCGGGCCACCCU |
| | | GAUGGACGAGCCCUACCGGAAGUCCAAGCUGACCUACGCCCAGGCCCGGAAGCUGCUGGGCCUGGAGGACACCGCCUUCUUCAAGGGCCUG |
| | | CGGUACGGCAAGGACAACGCCGAGGCCUCCACCCUGAUGGAGAUGAAGGCCUACCACGCCAUCUCCCGGGCCCUGGAGAAGGAGGGCCUGA |
| | | AGGACAAGAAGUCCCCCCUGAACCUGUCCUCCGAGCUGCAGGACGAGAUCGGCACCGCCUUCUCCCUGUUCAAGACCGACGAGGACAUCAC |
| | | CGGCCGGCUGAAGGACCGGGUGCAGCCCGAGAUCCUGGAGGCCCUGCUGAAGCACAUCUCCUUCGACAAGUUCGUGCAGAUCUCCCUGAAG |
| | | GCCCUGCGGCGGAUCGUGCCCCUGAUGGAGCAGGGCAAGCGGUACGACGAGGCCUGCGCCGAGAUCUACGGCGACCACUACGGCAAGAAGA |
| | | ACACCGAGGAGAAGAUCUACCUGCCCCCCAUCCCCGCCGACGAGAUCCGGAACCCCGUGGUGCUGCGGGCCCUGUCCCAGGCCCGGAAGGU |
| | | GAUCAACGGCGUGGUGCGGCGGUACGGCUCCCCCGCCCGGAUCCACAUCGAGACCGCCCGGGAGGUGGGCAAGUCCUUCAAGGACCGGAAG |
| | | GAGAUCGAGAAGCGGCAGGAGGAGAACCGGAAGGACCGGGAGAAGGCCGCCGCCAAGUUCCGGGAGUACUUCCCCAACUUCGUGGGCGAGC |
| | | CCAAGUCCAAGGACAUCCUGAAGCUGCGGCUGUACGAGCAGCAGCACGGCAAGUGCCUGUACUCCGGCAAGGAGAUCAACCUGGUGCGGCU |
| | | GAACGAGAAGGGCUACGUGGAGAUCGACCACGCCCUGCCCUUCUCCCGGACCUGGGACGACUCCUUCAACAACAAGGUGCUGGUGCUGGGC |
| | | UCCGAGAACCAGAACAAGGGCAACCAGACCCCCUACGAGUACUUCAACGGCAAGGACAACUCCCGGGAGUGGCAGGAGUUCAAGGCCCGGG |
| | | UGGAGACCUCCCGGUUCCCCCGGUCCAAGAAGCAGCGGAUCCUGCUGCAGAAGUUCGACGAGGACGGCUUCAAGGAGUGCAACCUGAACGA |
| | | CACCCGGUACGUGAACCGGUUCCUGUGCCAGUUCGUGGCCGACCACAUCCUGCUGACCGGCAAGGGCAAGCGGCGGGUGUUCGCCUCCAAC |
| | | GGCCAGAUCACCAACCUGCUGCGGGGCUUCUGGGGCCUGCGGAAGGUGCGGGCCGAGAACGACCGGCACCACGCCCUGGACGCCGUGGUGG |
| | | UGGCCUGCUCCACCGUGGCCAUGCAGCAGAAGAUCACCCGGUUCGUGCGGUACAAGGAGAUGAACGCCUUCGACGGCAAGACCAUCGACAA |
| | | GGAGACCGGCAAGGUGCUGCACCAGAAGACCCACUUCCCCCAGCCCUGGGAGUUCUUCGCCCAGGAGGUGAUGAUCCGGGUGUUCGGCAAG |
| | | CCCGACGGCAAGCCCGAGUUCGAGGAGGCCGACACCCCCGAGAAGCUGCGGACCCUGCUGGCCGAGAAGCUGUCCUCCCGGCCCGAGGCCG |
| | | UGCACGAGUACGUGACCCCCCUGUUCGUGUCCCGGGCCCCCAACCGGAAGAUGUCCGGCGCCCACAAGGACACCCUGCGGUCCGCCAAGCG |
| | | GUUCGUGAAGCACAACGAGAAGAUCUCCGUGAAGCGGGUGUGGCUGACCGAGAUCAAGCUGGCCGACCUGGAGAACAUGGUGAACUACAAG |
| | | AACGGCCGGGAGAUCGAGCUGUACGAGGCCCUGAAGGCCCGGCUGGAGGCCUACGGCGGCAACGCCAAGCAGGCCUUCGACCCCAAGGACA |
| | | ACCCCUUCUACAAGAAGGGCGGCCAGCUGGUGAAGGCCGUGCGGGUGGAGAAGACCCAGGAGUCCGGCGUGCUGCUGAACAAGAAGAACGC |
| | | CUACACCAUCGCCGACAACGGCGACAUGGUGCGGGUGGACGUGUUCUGCAAGGUGGACAAGAAGGGCAAGAACCAGUACUUCAUCGUGCCC |
| | | AUCUACGCCUGGCAGGUGGCCGAGAACAUCCUGCCCGACAUCGACUGCAAGGGCUACCGGAUCGACGACUCCUACACCUUCUGCUUCUCCC |
| | | UGCACAAGUACGACCUGAUCGCCUUCCAGAAGGACGAGAAGUCCAAGGUGGAGUUCGCCUACUACAUCAACUGCGACUCCUCCAACGGCCG |
| | | GUUCUACCUGGCCUGGCACGACAAGGGCUCCAAGGAGCAGCAGUUCCGGAUCUCCACCCAGAACCUGGUGCUGAUCCAGAAGUACCAGGUG |
| | | AACGAGCUGGGCAAGGAGAUCCGGCCCUGCCGGCUGAAGAAGCGGCCCCCCGUGCGGUCCGGCAAGCGGACCGCCGACGGCUCCGGCGGCG |
| | | GCUCCCCCAAGAAGAAGCGGAAGGUGGAGUAG |
| 34 | Nme2Cas9 | MIKIATRKYLGKQNVYDIGVERDHNFALKNGFIASNCWEGAPESATESGGTSTESEGSAGTSTESEGSAGSAGSTSGGSGGGSGGSGSAGT |
| | Intein | STESEGSAGTSESATESGGTSTESEGSSSGAAFKPNPINYILGLDIGIASVGWAMVEIDEEENPIRLIDLGVRVFERAEVPKTGDSLAMAR |
| | (mRNA J)- | RLARSVRRLTRRRAHRLLRARRLLKREGVLQAADFDENGLIKSLPNTPWQLRAAALDRKLTPLEWSAVLLHLI KHRGYLSQRKNEGETADK |
| | amino | ELGALLKGVANNAHALQTGDFRTPAELALNKFEKESGHIRNQRGDYSHTFSRKDLQAELILLFEKQKEFGNPHVSGGLKEGIETLLMTQRP |
| | acid | ALSGDAVQKMLGHCTFEPAEPKAAKNTYTAERFIWLTKLNNLRILEQGSERPLTDTERATLMDEPYRKSKLTYAQARKLLGLEDTAFFKGL |
| | sequence | RYGKDNAEASTLMEMKAYHAISRALEKEGLKDKKSPLNLSSELQDEIGTAFSLFKTDEDITGRLKDRVQPEILEALLKHISFDKFVQISLK |
| | | ALRRIVPLMEQGKRYDEACAEIYGDHYGKKNTEEKIYLPPIPADEIRNPVVLRALSQARKVINGVVRRYGSPARIHIETAREVGKSFKDRK |
| | | EIEKRQEENRKDREKAAAKFREYFPNFVGEPKSKDILKLRLYEQQHGKCLYSGKEINLVRLNEKGYVEIDHALPFSRTWDDSFNNKVLVLG |
| | | SENQNKGNQTPYEYFNGKDNSREWQEFKARVETSRFPRSKKQRILLQKFDEDGFKECNLNDTRYVNRFLCQFVADHILLTGKGKRRVFASN |
| | | GQITNLLRGFWGLRKVRAENDRHHALDAVVVACSTVAMQQKITRFVRYKEMNAFDGKTIDKETGKVLHQKTHFPQPWEFFAQEVMIRVFGK |
| | | PDGKPEFEEADTPEKLRTLLAEKLSSRPEAVHEYVTPLFVSRAPNRKMSGAHKDTLRSAKRFVKHNEKISVKRVWLTEIKLADLENMVNYK |
| | | NGREIELYEALKARLEAYGGNAKQAFDPKDNPFYKKGGQLVKAVRVEKTQESGVLLNKKNAYTIADNGDMVRVDVFCKVDKKGKNQYFIVP |
| | | IYAWQVAENILPDIDCKGYRIDDSYTFCFSLHKYDLIAFQKDEKSKVEFAYYINCDSSNGRFYLAWHDKGSKEQQFRISTQNLVLIQKYQV |
| | | NELGKEIRPCRLKKRPPVRSGKRTADGSGGGSPKKKRKVE* |
| 35 | SpyCas9 | GGGAAGCUCAGAAUAAACGCUCAACUUUGGCCGGAUCUGCCACCAUGGACAAGAAGUACUCCAUCGGCCUGGACAUCGGCACCAACUCCGU |
| | with | GGGCUGGGCCGUGAUCACCGACGAGUACAAGGUGCCCUCCAAGAAGUUCAAGGUGCUGGGCAACACCGACCGGCACUCCAUCAAGAAGAAC |
| | HiBit | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | tag | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | (mRNA K)- | GGAGGAGGACAAGAAGCACGAGCGGCACCCCAUCUUCGGCAACAUCGUGGACGAGGUGGCCUACCACGAGAAGUACCCCACCAUCUACCAC |
| | mRNA | CUGCGGAAGAAGCUGGUGGACUCCACCGACAAGGCCGACCUGCGGCUGAUCUACCUGGCCCUGGCCCACAUGAUCAAGUUCCGGGGCCACU |
| | sequence | UCCUGAUCGAGGGCGACCUGAACCCCGACAACUCCGACGUGGACAAGCUGUUCAUCCAGCUGGUGCAGACCUACAACCAGCUGUUCGAGGA |
| | | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | | CCGAGGACGCCAAGCUGCAGCUGUCCAAGGACACCUACGACGACGACCUGGACAACCUGCUGGCCCAGAUCGGCGACCAGUACGCCGACCU |
| | | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | | UCCAUGAUCAAGCGGUACGACGAGCACCACCAGGACCUGACCCUGCUGAAGGCCCUGGUGCGGCAGCAGCUGCCCGAGAAGUACAAGGAGA |
| | | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGGAGGACCUGCUGCGGAAGCAGCGGACCUUCGACAACGGCUCCAUC |
| | | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACCGGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGCGGCGACGGCGGCGGCUCCCCCAAGAAGAAGCGGAAGGUGUCCGA |
| | | GUCCGCCACCCCCGAGUCCGUGUCCGGCUGGCGGCUGUUCAAGAAGAUCUCCUGACUAGCACCAGCCUCAAGAACACCCGAAUGGAGUCUC |
| | | UAAGCUACAUAAUACCAACUUACACUUUACAAAAUGUUGUCCCCCAAAAUGUAGCCAUUCGUAUCUGCUCCUAAUAAAAAGAAAGUUUCUU |
| | | CACAUUCUCUCGAGAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAUCUAG |
| 36 | SpyCas9 | auggacaagaaguacuccaucggccuggacaucggcaccaacuccgugggcugggccgugaucaccgacgaguacaaggugcccuccaaga |
| | with | aguucaaggugcugggcaacaccgaccggcacuccaucaagaagaaccugaucggcgcccugcuguucgacuccggcgagaccgccgaggc |
| | HiBit | cacccggcugaagcggaccgcccggcggcgguacacccggcggaagaaccggaucugcuaccugcaggagaucuucuccaacgagauggcc |
| | tag | aagguggacgacuccuucuuccaccggcuggaggaguccuuccugguggaggaggacaagaagcacgagcggcaccccaucuucggcaaca |
| | (mRNA K)- | ucguggacgagguggccuaccacgagaaguaccccaccaucuaccaccugcggaagaagcugguggacuccaccgacaaggccgaccugcg |
| | ORF | gcugaucuaccuggcccuggcccacaugaucaaguuccggggccacuuccugaucgaggcggaccugaaccccgacaacuccgacguggac |
| | sequence | aagcuguucauccagcuggugcagaccuacaaccagcuguucgaggagaaccccaucaacgccuccggcguggacgccaaggccauccugu |
| | | ccgcccggcuguccaagucccggcggcuggagaaccugaucgcccagcugcccggcgagaagaagaacggccuguucggcaaccugaucgc |
| | | ccugucccugggccugacccccaacuucaaguccaacuucgaccuggccgaggacgccaagcugcagcuguccaaggacaccuacgacgac |
| | | gaccuggacaaccugcuggcccagaucggcgaccaguacgccgaccuguuccuggccgccaagaaccuguccgacgccauccugcuguccg |
| | | acauccugcgggugaacaccgagaucaccaaggccccccuguccgccuccaugaucaagcgguacgacgagcaccaccaggaccugacccu |
| | | gcugaaggcccuggugcggcagcagcugcccgagaaguacaaggagaucuucuucgaccaguccaagaacggcuacgccggcuacaucgac |
| | | ggcggcgccucccaggaggaguucuacaaguucaucaagcccauccuggagaagauggacggcaccgaggagcugcuggugaagcugaacc |
| | | gggaggaccugcugcggaagcagcggaccuucgacaacggcuccaucccccaccagauccaccugggcgagcugcacgccauccugcggcg |
| | | gcaggaggacuucuaccccuuccugaaggacaaccgggagaagaucgagaagauccugaccuuccggauccccuacuacgugggcccccug |
| | | gcccggggcaacucccgguucgccuggaugacccggaaguccgaggagaccaucacccccuggaacuucgaggaggugguggacaagggcg |
| | | ccuccgcccaguccuucaucgagcggaugaccaacuucgacaagaaccugcccaacgagaaggugcugcccaagcacucccugcuguacga |
| | | guacuucaccguguacaacgagcugaccaaggugaaguacgugaccgagggcaugcggaagcccgccuuccuguccggcgagcagaagaag |
| | | gccaucguggaccugcuguucaagaccaaccggaaggugaccgugaagcagcugaaggaggacuacuucaagaagaucgagugcuucgacu |
| | | ccguggagaucuccggcguggaggaccgguucaacgccucccugggcaccuaccacgaccugcugaagaucaucaaggacaaggacuuccu |
| | | ggacaacgaggagaacgaggacauccuggaggacaucgugcugacccugacccuguucgaggaccgggagaugaucgaggagcggcugaag |
| | | accuacgcccaccuguucgacgacaaggugaugaagcagcugaagcggcggcgguacaccggcuggggccggcugucccggaagcugauca |
| | | acggcauccgggacaagcaguccggcaagaccauccuggacuuccugaaguccgacggcuucgccaaccggaacuucaugcagcugaucca |
| | | cgacgacucccugaccuucaaggaggacauccagaaggcccagguguccggccagggcgacucccugcacgagcacaucgccaaccuggcc |
| | | ggcucccccgccaucaagaagggcauccugcagaccgugaaggugguggacgagcuggugaaggugaugggccggcacaagcccgagaaca |
| | | ucgugaucgagauggcccgggagaaccagaccacccagaagggccagaagaacucccgggagcggaugaagcggaucgaggagggcaucaa |
| | | ggagcugggcucccagauccugaaggagcaccccguggagaacacccagcugcagaacgagaagcuguaccuguacuaccugcagaacggc |
| | | cgggacauguacguggaccaggagcuggacaucaaccggcuguccgacuacgacguggaccacaucgugccccaguccuuccugaaggacg |
| | | acuccaucgacaacaaggugcugacccgguccgacaagaaccggggcaaguccgacaacgugcccuccgaggagguggugaagaagaugaa |
| | | gaacuacuggcggcagcugcugaacgccaagcugaucacccagcggaaguucgacaaccugaccaaggccgagcggcggggccuguccgag |
| | | cuggacaaggccggcuucaucaagcggcagcugguggagacccggcagaucaccaagcacguggcccagauccuggacucccggaugaaca |
| | | ccaaguacgacgagaacgacaagcugauccgggaggugaaggugaucacccugaaguccaagcugguguccgacuuccggaaggacuucca |
| | | guucuacaaggugcgggagaucaacaacuaccaccacgcccacgacgccuaccugaacgccguggugggcaccgcccugaucaagaaguac |
| | | cccaagcuggaguccgaguucguguacggcgacuacaagguguacgacgugcggaagaugaucgccaaguccgagcaggagaucggcaagg |
| | | ccaccgccaaguacuucuucuacuccaacaucaugaacuucuucaagaccgagaucacccuggccaacggcgagauccggaagcggccccu |
| | | gaucgagaccaacggcgagaccggcgagaucgugugggacaagggccgggacuucgccaccgugcggaaggugcuguccaugccccaggug |
| | | aacaucgugaagaagaccgaggugcagaccggcggcuucuccaaggaguccauccugcccaagcggaacuccgacaagcugaucgcccgga |
| | | agaaggacugggaccccaagaaguacggcggcuucgacucccccaccguggccuacuccgugcuggugguggccaagguggagaagggcaa |
| | | guccaagaagcugaaguccgugaaggagcugcugggcaucaccaucauggagcgguccuccuucgagaagaaccccaucgacuuccuggag |
| | | gccaagggcuacaaggaggugaagaaggaccugaucaucaagcugcccaaguacucccuguucgagcuggagaacggccggaagcggaugc |
| | | uggccuccgccggcgagcugcagaagggcaacgagcuggcccugcccuccaaguacgugaacuuccuguaccuggccucccacuacgagaa |
| | | gcugaagggcucccccgaggacaacgagcagaagcagcuguucguggagcagcacaagcacuaccuggacgagaucaucgagcagaucucc |
| | | gaguucuccaagcgggugauccuggccgacgccaaccuggacaaggugcuguccgccuacaacaagcaccgggacaagcccauccgggagc |
| | | aggccgagaacaucauccaccuguucacccugaccaaccugcgggcccccgccgccuucaaguacuucgacaccaccaucgaccggaagcg |
| | | guacaccuccaccaaggaggugcuggacgccacccugauccaccaguccaucaccggccuguacgagacccggaucgaccugucccagcug |
| | | ggcggcgacggcggcggcucccccaagaagaagcggaagguguccgaguccgccacccccgaguccguguccggcuggcggcuguucaaga |
| | | agaucuccuga |
| 37 | SpyCas9 | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | with | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | HiBit | KLFIQLVQTYNQLFEENPINASGVDAKAILSARLSKSRRLENLIAQLPGEKKNGLFGNLIALSLGLTPNFKSNFDLAEDAKLQLSKDTYDD |
| | tag | DLDNLLAQIGDQYADLFLAAKNLSDAILLSDILRVNTEITKAPLSASMIKRYDEHHQDLTLLKALVRQQLPEKYKEIFFDQSKNGYAGYID |
| | (mRNA K)- | GGASQEEFYKFIKPILEKMDGTEELLVKLNREDLLRKQRTFDNGSIPHQIHLGELHAILRRQEDFYPFLKDNREKIEKILTFRIPYYVGPL |
| | amino | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | acid | AIVDLLFKTNRKVTVKQLKEDYFKKIECFDSVEISGVEDRFNASLGTYHDLLKIIKDKDFLDNEENEDILEDIVLTLTLFEDREMIEERLK |
| | sequence | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKRYTSTKEVLDATLIHQSITGLYETRIDLSQL |
| | | GGDGGGSPKKKRKVSESATPESVSGWRLFKKIS* |
| 38 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | (mRNA L)- | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | mRNA | GGAGGAGGACAAGAAGCACGAGCGGCACCCCAUCUUCGGCAACAUCGUGGACGAGGUGGCCUACCACGAGAAGUACCCCACCAUCUACCAC |
| | sequence | CUGCGGAAGAAGCUGGUGGACUCCACCGACAAGGCCGACCUGCGGCUGAUCUACCUGGCCCUGGCCCACAUGAUCAAGUUCCGGGGCCACU |
| | | UCCUGAUCGAGGGCGACCUGAACCCCGACAACUCCGACGUGGACAAGCUGUUCAUCCAGCUGGUGCAGACCUACAACCAGCUGUUCGAGGA |
| | | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | | CCGAGGACGCCAAGCUGCAGCUGUCCAAGGACACCUACGACGACGACCUGGACAACCUGCUGGCCCAGAUCGGCGACCAGUACGCCGACCU |
| | | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | | UCCAUGAUCAAGCGGUACGACGAGCACCACCAGGACCUGACCCUGCUGAAGGCCCUGGUGCGGCAGCAGCUGCCCGAGAAGUACAAGGAGA |
| | | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGGAGGACCUGCUGCGGAAGCAGCGGACCUUCGACAACGGCUCCAUC |
| | | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCGGCGGCUCCGGCGGCGGC |
| | | UCCGGCGGCUCCGGCUCCGCCGGCACCUCCACCGAGUCCGAGGGCUCCGCCGGCACCUCCGAGUCCGCCACCGAGUCCGGCGGCACCUCCA |
| | | CCGAGUCCGAGGGCUCCUCCUCCGGCGCCGCCUUCAAGCCCAACCCCAUCAACUACAUCCUGGGCCUGGACAUCGGCAUCGCCUCCGUGGG |
| | | CUGGGCCAUGGUGGAGAUCGACGAGGAGGAGAACCCCAUCCGGCUGAUCGACCUGGGCGUGCGGGUGUUCGAGCGGGCCGAGGUGCCCAAG |
| | | ACCGGCGACUCCCUGGCCAUGGCCCGGCGGCUGGCCCGGUCCGUGCGGCGGCUGACCCGGCGGCGGGCCCACCGGCUGCUGCGGGCCCGGC |
| | | GGCUGCUGAAGCGGGAGGGCGUGCUGCAGGCCGCCGACUUCGACGAGAACGGCCUGAUCAAGUCCCUGCCCAACACCCCCUGGCAGCUGCG |
| | | GGCCGCCGCCCUGGACCGGAAGCUGACCCCCCUGGAGUGGUCCGCCGUGCUGCUGCACCUGAUCAAGCACCGGGGCUACCUGUCCCAGCGG |
| | | AAGAACGAGGGCGAGACCGCCGACAAGGAGCUGGGCGCCCUGCUGAAGGGCGUGGCCAACAACGCCCACGCCCUGCAGACCGGCGACUUCC |
| | | GGACCCCCGCCGAGCUGGCCCUGAACAAGUUCGAGAAGGAGUCCGGCCACAUCCGGAACCAGCGGGGCGACUACUCCCACACCUUCUCCCG |
| | | GAAGGACCUGCAGGCCGAGCUGAUCCUGCUGUUCGAGAAGCAGAAGGAGUUCGGCAACCCCCACGUGUCCGGCGGCCUGAAGGAGGGCAUC |
| | | GAGACCCUGCUGAUGACCCAGCGGCCCGCCCUGUCCGGCGACGCCGUGCAGAAGAUGCUGGGCCACUGCACCUUCGAGCCCGCCGAGCCCA |
| | | AGGCCGCCAAGAACACCUACACCGCCGAGCGGUUCAUCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAGCUGACCUACGCCCAGGCCCGGAAGCUGCUGGGCCUG |
| | | GAGGACACCGCCUUCUUCAAGGGCCUGCGGUACGGCAAGGACAACGCCGAGGCCUCCACCCUGAUGGAGAUGAAGGCCUACCACGCCAUCU |
| | | CCCGGGCCCUGGAGAAGGAGGGCCUGAAGGACAAGAAGUCCCCCCUGAACCUGUCCUCCGAGCUGCAGGACGAGAUCGGCACCGCCUUCUC |
| | | CCUGUUCAAGACCGACGAGGACAUCACCGGCCGGCUGAAGGACCGGGUGCAGCCCGAGAUCCUGGAGGCCCUGCUGAAGCACAUCUCCUUC |
| | | GACAAGUUCGUGCAGAUCUCCCUGAAGGCCCUGCGGCGGAUCGUGCCCCUGAUGGAGCAGGGCAAGCGGUACGACGAGGCCUGCGCCGAGA |
| | | UCUACGGCGACCACUACGGCAAGAAGAACACCGAGGAGAAGAUCUACCUGCCCCCCAUCCCCGCCGACGAGAUCCGGAACCCCGUGGUGCU |
| | | GCGGGCCCUGUCCCAGGCCCGGAAGGUGAUCAACGGCGUGGUGCGGCGGUACGGCUCCCCCGCCCGGAUCCACAUCGAGACCGCCCGGGAG |
| | | GUGGGCAAGUCCUUCAAGGACCGGAAGGAGAUCGAGAAGCGGCAGGAGGAGAACCGGAAGGACCGGGAGAAGGCCGCCGCCAAGUUCCGGG |
| | | AGUACUUCCCCAACUUCGUGGGCGAGCCCAAGUCCAAGGACAUCCUGAAGCUGCGGCUGUACGAGCAGCAGCACGGCAAGUGCCUGUACUC |
| | | CGGCAAGGAGAUCAACCUGGUGCGGCUGAACGAGAAGGGCUACGUGGAGAUCGACCACGCCCUGCCCUUCUCCCGGACCUGGGACGACUCC |
| | | UUCAACAACAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUCGACGAGGA |
| | | CGGCUUCAAGGAGUGCAACCUGAACGACACCCGGUACGUGAACCGGUUCCUGUGCCAGUUCGUGGCCGACCACAUCCUGCUGACCGGCAAG |
| | | GGCAAGCGGCGGGUGUUCGCCUCCAACGGCCAGAUCACCAACCUGCUGCGGGGCUUCUGGGGCCUGCGGAAGGUGCGGGCCGAGAACGACC |
| | | GGCACCACGCCCUGGACGCCGUGGUGGUGGCCUGCUCCACCGUGGCCAUGCAGCAGAAGAUCACCCGGUUCGUGCGGUACAAGGAGAUGAA |
| | | CGCCUUCGACGGCAAGACCAUCGACAAGGAGACCGGCAAGGUGCUGCACCAGAAGACCCACUUCCCCCAGCCCUGGGAGUUCUUCGCCCAG |
| | | GAGGUGAUGAUCCGGGUGUUCGGCAAGCCCGACGGCAAGCCCGAGUUCGAGGAGGCCGACACCCCCGAGAAGCUGCGGACCCUGCUGGCCG |
| | | AGAAGCUGUCCUCCCGGCCCGAGGCCGUGCACGAGUACGUGACCCCCCUGUUCGUGUCCCGGGCCCCCAACCGGAAGAUGUCCGGCGCCCA |
| | | CAAGGACACCCUGCGGUCCGCCAAGCGGUUCGUGAAGCACAACGAGAAGAUCUCCGUGAAGCGGGUGUGGCUGACCGAGAUCAAGCUGGCC |
| | | GACCUGGAGAACAUGGUGAACUACAAGAACGGCCGGGAGAUCGAGCUGUACGAGGCCCUGAAGGCCCGGCUGGAGGCCUACGGCGGCAACG |
| | | CCAAGCAGGCCUUCGACCCCAAGGACAACCCCUUCUACAAGAAGGGCGGCCAGCUGGUGAAGGCCGUGCGGGUGGAGAAGACCCAGGAGUC |
| | | CGGCGUGCUGCUGAACAAGAAGAACGCCUACACCAUCGCCGACAACGGCGACAUGGUGCGGGUGGACGUGUUCUGCAAGGUGGACAAGAAG |
| | | GGCAAGAACCAGUACUUCAUCGUGCCCAUCUACGCCUGGCAGGUGGCCGAGAACAUCCUGCCCGACAUCGACUGCAAGGGCUACCGGAUCG |
| | | ACGACUCCUACACCUUCUGCUUCUCCCUGCACAAGUACGACCUGAUCGCCUUCCAGAAGGACGAGAAGUCCAAGGUGGAGUUCGCCUACUA |
| | | CAUCAACUGCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCGGUCCGGCA |
| | | AGCGGACCGCCGACGGCUCCGGCGGCGGCUCCCCCAAGAAGAAGCGGAAGGUGGAGUAGCUAGCACCAGCCUCAAGAACACCCGAAUGGAG |
| | | UCUCUAAGCUACAUAAUACCAACUUACACUUUACAAAAUGUUGUCCCCCAAAAUGUAGCCAUUCGUAUCUGCUCCUAAUAAAAAGAAAGUU |
| | | UCUUCACAUUCUCUCGAGAAAAAAAAAAAAUGGAAAAAAAAAAAACGGAAAAAAAAAAAAGGUAAAAAAAAAAAAUAUAAAAAAAAAAAAC |
| | | AUAAAAAAAAAAAACGAAAAAAAAAAAACGUAAAAAAAAAAAACUCAAAAAAAAAAAGAUAAAAAAAAAAAACCUAAAAAAAAAAAAUGUA |
| | | AAAAAAAAAAAGGGAAAAAAAAAAAACGCAAAAAAAAAAAACACAAAAAAAAAAAAUGCAAAAAAAAAAAAUCGAAAAAAAAAAAAUCUAA |
| | | AAAAAAAAAACGAAAAAAAAAAAACCCAAAAAAAAAAAAGACAAAAAAAAAAAAUAGAAAAAAAAAAAGUUAAAAAAAAAAAACUGAAAAA |
| | | AAAAAAAUUUAAAAAAAAAAAAUCUAG |
| 39 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion | CACCCGGCUGAAGCGGACCGCCCGGCGGCGGUACACCCGGCGGAAGAACCGGAUCUGCUACCUGCAGGAGAUCUUCUCCAACGAGAUGGCC |
| | (mRNA L)- | AAGGUGGACGACUCCUUCUUCCACCGGCUGGAGGAGUCCUUCCUGGUGGAGGAGGACAAGAAGCACGAGCGGCACCCCAUCUUCGGCAACA |
| | ORF | UCGUGGACGAGGUGGCCUACCACGAGAAGUACCCCACCAUCUACCACCUGCGGAAGAAGCUGGUGGACUCCACCGACAAGGCCGACCUGCG |
| | sequence | GCUGAUCUACCUGGCCCUGGCCCACAUGAUCAAGUUCCGGGGCCACUUCCUGAUCGAGGGCGACCUGAACCCCGACAACUCCGACGUGGAC |
| | | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | | CCUGUCCCUGGGCCUGACCCCCAACUUCAAGUCCAACUUCGACCUGGCCGAGGACGCCAAGCUGCAGCUGUCCAAGGACACCUACGACGAC |
| | | GACCUGGACAACCUGCUGGCCCAGAUCGGCGACCAGUACGCCGACCUGUUCCUGGCCGCCAAGAACCUGUCCGACGCCAUCCUGCUGUCCG |
| | | ACAUCCUGCGGGUGAACACCGAGAUCACCAAGGCCCCCCUGUCCGCCUCCAUGAUCAAGCGGUACGACGAGCACCACCAGGACCUGACCCU |
| | | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCGGCGGCUCCGGCGGCGGCUCCGGCGGCUCCGGCUCCGCCGGCACCUCCACCGAGUCCGAGGG |
| | | CUCCGCCGGCACCUCCGAGUCCGCCACCGAGUCCGGCGGCACCUCCACCGAGUCCGAGGGCUCCUCCUCCGGCGCCGCCUUCAAGCCCAAC |
| | | CCCAUCAACUACAUCCUGGGCCUGGACAUCGGCAUCGCCUCCGUGGGCUGGGCCAUGGUGGAGAUCGACGAGGAGGAGAACCCCAUCCGGC |
| | | UGAUCGACCUGGGCGUGCGGGUGUUCGAGCGGGCCGAGGUGCCCAAGACCGGCGACUCCCUGGCCAUGGCCCGGCGGCUGGCCCGGUCCGU |
| | | GCGGCGGCUGACCCGGCGGCGGGCCCACCGGCUGCUGCGGGCCCGGCGGCUGCUGAAGCGGGAGGGCGUGCUGCAGGCCGCCGACUUCGAC |
| | | GAGAACGGCCUGAUCAAGUCCCUGCCCAACACCCCCUGGCAGCUGCGGGCCGCCGCCCUGGACCGGAAGCUGACCCCCCUGGAGUGGUCCG |
| | | CCGUGCUGCUGCACCUGAUCAAGCACCGGGGCUACCUGUCCCAGCGGAAGAACGAGGGCGAGACCGCCGACAAGGAGCUGGGCGCCCUGCU |
| | | GAAGGGCGUGGCCAACAACGCCCACGCCCUGCAGACCGGCGACUUCCGGACCCCCGCCGAGCUGGCCCUGAACAAGUUCGAGAAGGAGUCC |
| | | GGCCACAUCCGGAACCAGCGGGGCGACUACUCCCACACCUUCUCCCGGAAGGACCUGCAGGCCGAGCUGAUCCUGCUGUUCGAGAAGCAGA |
| | | AGGAGUUCGGCAACCCCCACGUGUCCGGCGGCCUGAAGGAGGGCAUCGAGACCCUGCUGAUGACCCAGCGGCCCGCCCUGUCCGGCGACGC |
| | | CGUGCAGAAGAUGCUGGGCCACUGCACCUUCGAGCCCGCCGAGCCCAAGGCCGCCAAGAACACCUACACCGCCGAGCGGUUCAUCUGGCUG |
| | | ACCAAGCUGAACAACCUGCGGAUCCUGGAGCAGGGCUCCGAGCGGCCCCUGACCGACACCGAGCGGGCCACCCUGAUGGACGAGCCCUACC |
| | | GGAAGUCCAAGCUGACCUACGCCCAGGCCCGGAAGCUGCUGGGCCUGGAGGACACCGCCUUCUUCAAGGGCCUGCGGUACGGCAAGGACAA |
| | | CGCCGAGGCCUCCACCCUGAUGGAGAUGAAGGCCUACCACGCCAUCUCCCGGGCCCUGGAGAAGGAGGGCCUGAAGGACAAGAAGUCCCCC |
| | | CUGAACCUGUCCUCCGAGCUGCAGGACGAGAUCGGCACCGCCUUCUCCCUGUUCAAGACCGACGAGGACAUCACCGGCCGGCUGAAGGACC |
| | | GGGUGCAGCCCGAGAUCCUGGAGGCCCUGCUGAAGCACAUCUCCUUCGACAAGUUCGUGCAGAUCUCCCUGAAGGCCCUGCGGCGGAUCGU |
| | | GCCCCUGAUGGAGCAGGGCAAGCGGUACGACGAGGCCUGCGCCGAGAUCUACGGCGACCACUACGGCAAGAAGAACACCGAGGAGAAGAUC |
| | | UACCUGCCCCCCAUCCCCGCCGACGAGAUCCGGAACCCCGUGGUGCUGCGGGCCCUGUCCCAGGCCCGGAAGGUGAUCAACGGCGUGGUGC |
| | | GGCGGUACGGCUCCCCCGCCCGGAUCCACAUCGAGACCGCCCGGGAGGUGGGCAAGUCCUUCAAGGACCGGAAGGAGAUCGAGAAGCGGCA |
| | | GGAGGAGAACCGGAAGGACCGGGAGAAGGCCGCCGCCAAGUUCCGGGAGUACUUCCCCAACUUCGUGGGCGAGCCCAAGUCCAAGGACAUC |
| | | CUGAAGCUGCGGCUGUACGAGCAGCAGCACGGCAAGUGCCUGUACUCCGGCAAGGAGAUCAACCUGGUGCGGCUGAACGAGAAGGGCUACG |
| | | UGGAGAUCGACCACGCCCUGCCCUUCUCCCGGACCUGGGACGACUCCUUCAACAACAAGGUGCUGGUGCUGGGCUCCGAGAACCAGAACAA |
| | | GGGCAACCAGACCCCCUACGAGUACUUCAACGGCAAGGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCAAGGAGUGCAACCUGAACGACACCCGGUACGUGAACC |
| | | GGUUCCUGUGCCAGUUCGUGGCCGACCACAUCCUGCUGACCGGCAAGGGCAAGCGGCGGGUGUUCGCCUCCAACGGCCAGAUCACCAACCU |
| | | GCUGCGGGGCUUCUGGGGCCUGCGGAAGGUGCGGGCCGAGAACGACCGGCACCACGCCCUGGACGCCGUGGUGGUGGCCUGCUCCACCGUG |
| | | GCCAUGCAGCAGAAGAUCACCCGGUUCGUGCGGUACAAGGAGAUGAACGCCUUCGACGGCAAGACCAUCGACAAGGAGACCGGCAAGGUGC |
| | | UGCACCAGAAGACCCACUUCCCCCAGCCCUGGGAGUUCUUCGCCCAGGAGGUGAUGAUCCGGGUGUUCGGCAAGCCCGACGGCAAGCCCGA |
| | | GUUCGAGGAGGCCGACACCCCCGAGAAGCUGCGGACCCUGCUGGCCGAGAAGCUGUCCUCCCGGCCCGAGGCCGUGCACGAGUACGUGACC |
| | | CCCCUGUUCGUGUCCCGGGCCCCCAACCGGAAGAUGUCCGGCGCCCACAAGGACACCCUGCGGUCCGCCAAGCGGUUCGUGAAGCACAACG |
| | | AGAAGAUCUCCGUGAAGCGGGUGUGGCUGACCGAGAUCAAGCUGGCCGACCUGGAGAACAUGGUGAACUACAAGAACGGCCGGGAGAUCGA |
| | | GCUGUACGAGGCCCUGAAGGCCCGGCUGGAGGCCUACGGCGGCAACGCCAAGCAGGCCUUCGACCCCAAGGACAACCCCUUCUACAAGAAG |
| | | GGCGGCCAGCUGGUGAAGGCCGUGCGGGUGGAGAAGACCCAGGAGUCCGGCGUGCUGCUGAACAAGAAGAACGCCUACACCAUCGCCGACA |
| | | ACGGCGACAUGGUGCGGGUGGACGUGUUCUGCAAGGUGGACAAGAAGGGCAAGAACCAGUACUUCAUCGUGCCCAUCUACGCCUGGCAGGU |
| | | GGCCGAGAACAUCCUGCCCGACAUCGACUGCAAGGGCUACCGGAUCGACGACUCCUACACCUUCUGCUUCUCCCUGCACAAGUACGACCUG |
| | | AUCGCCUUCCAGAAGGACGAGAAGUCCAAGGUGGAGUUCGCCUACUACAUCAACUGCGACUCCUCCAACGGCCGGUUCUACCUGGCCUGGC |
| | | ACGACAAGGGCUCCAAGGAGCAGCAGUUCCGGAUCUCCACCCAGAACCUGGUGCUGAUCCAGAAGUACCAGGUGAACGAGCUGGGCAAGGA |
| | | GAUCCGGCCCUGCCGGCUGAAGAAGCGGCCCCCCGUGCGGUCCGGCAAGCGGACCGCCGACGGCUCCGGCGGCGGCUCCCCCAAGAAGAAG |
| | | CGGAAGGUGGAGUAG |
| 40 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion | KLFIQLVQTYNQLFEENPINASGVDAKAILSARLSKSRRLENLIAQLPGEKKNGLFGNLIALSLGLTPNFKSNFDLAEDAKLQLSKDTYDD |
| | (mRNA L)- | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | sequence | AIVDLLFKTNRKVTVKQLKEDYFKKIECFDSVEISGVEDRFNASLGTYHDLLKIIKDKDFLDNEENEDILEDIVLTLTLFEDREMIEERLK |
| | | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSGGSGGGSGGSGSAGTSTESEGSAGTSESATESGGTSTESEGSSSGAAFKPN |
| | | PINYILGLDIGIASVGWAMVEIDEEENPIRLIDLGVRVFERAEVPKTGDSLAMARRLARSVRRLTRRRAHRLLRARRLLKREGVLQAADFD |
| | | ENGLIKSLPNTPWQLRAAALDRKLTPLEWSAVLLHLIKHRGYLSQRKNEGETADKELGALLKGVANNAHALQTGDFRTPAELALNKFEKES |
| | | GHIRNQRGDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLVRLNEKGYVEIDHALPFSRTWDDSFNNKVLVLGSENQNKGNQTPYEYFNGKDNSREWQEFKARVETSRF |
| | | PRSKKQRILLQKFDEDGFKECNLNDTRYVNRFLCQFVADHILLTGKGKRRVFASNGQITNLLRGFWGLRKVRAENDRHHALDAVVVACSTV |
| | | AMQQKITRFVRYKEMNAFDGKTIDKETGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEALKARLEAYGGNAKQAFDPKDNPFYKK |
| | | GGQLVKAVRVEKTQESGVLLNKKNAYTIADNGDMVRVDVFCKVDKKGKNQYFIVPIYAWQVAENILPDIDCKGYRIDDSYTFCFSLHKYDL |
| | | IAFQKDEKSKVEFAYYINCDSSNGRFYLAWHDKGSKEQQFRISTQNLVLIQKYQVNELGKEIRPCRLKKRPPVRSGKRTADGSGGGSPKKK |
| | | RKVE* |
| 41 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | with | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | HiBit | GGAGGAGGACAAGAAGCACGAGCGGCACCCCAUCUUCGGCAACAUCGUGGACGAGGUGGCCUACCACGAGAAGUACCCCACCAUCUACCAC |
| | tag | CUGCGGAAGAAGCUGGUGGACUCCACCGACAAGGCCGACCUGCGGCUGAUCUACCUGGCCCUGGCCCACAUGAUCAAGUUCCGGGGCCACU |
| | (mRNA M)- | UCCUGAUCGAGGGCGACCUGAACCCCGACAACUCCGACGUGGACAAGCUGUUCAUCCAGCUGGUGCAGACCUACAACCAGCUGUUCGAGGA |
| | mRNA | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | sequence | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | | CCGAGGACGCCAAGCUGCAGCUGUCCAAGGACACCUACGACGACGACCUGGACAACCUGCUGGCCCAGAUCGGCGACCAGUACGCCGACCU |
| | | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | | UCCAUGAUCAAGCGGUACGACGAGCACCACCAGGACCUGACCCUGCUGAAGGCCCUGGUGCGGCAGCAGCUGCCCGAGAAGUACAAGGAGA |
| | | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | | GGAGAAGAUGGACGGCACCGAGGAGCUGCUGGUGAAGCUGAACCGGGAGGACCUGCUGCGGAAGCAGCGGACCUUCGACAACGGCUCCAUC |
| | | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCACCGAGGAGGGCACCUCC |
| | | ACCGAGUCCGAGGGCUCCGCCGGCACCUCCACCGAGUCCGAGGGCUCCGCCGGCACCUCCGAGUCCGCCACCGAGUCCGGCGGCACCUCCA |
| | | CCGAGUCCGAGGGCUCCUCCUCCGGUGCCGCCUUCAAGCCCAACCCCAUCAACUACAUCCUGGGCCUGGACAUCGGCAUCGCCUCCGUGGG |
| | | CUGGGCCAUGGUGGAGAUCGACGAGGAGGAGAACCCCAUCCGGCUGAUCGACCUGGGCGUGCGGGUGUUCGAGCGGGCCGAGGUGCCCAAG |
| | | ACCGGCGACUCCCUGGCCAUGGCCCGGCGGCUGGCCCGGUCCGUGCGGCGGCUGACCCGGCGGCGGGCCCACCGGCUGCUGCGGGCCCGGC |
| | | GGCUGCUGAAGCGGGAGGGCGUGCUGCAGGCCGCCGACUUCGACGAGAACGGCCUGAUCAAGUCCCUGCCCAACACCCCCUGGCAGCUGCG |
| | | GGCCGCCGCCCUGGACCGGAAGCUGACCCCCCUGGAGUGGUCCGCCGUGCUGCUGCACCUGAUCAAGCACCGGGGCUACCUGUCCCAGCGG |
| | | AAGAACGAGGGCGAGACCGCCGACAAGGAGCUGGGCGCCCUGCUGAAGGGCGUGGCCAACAACGCCCACGCCCUGCAGACCGGCGACUUCC |
| | | GGACCCCCGCCGAGCUGGCCCUGAACAAGUUCGAGAAGGAGUCCGGCCACAUCCGGAACCAGCGGGGCGACUACUCCCACACCUUCUCCCG |
| | | GAAGGACCUGCAGGCCGAGCUGAUCCUGCUGUUCGAGAAGCAGAAGGAGUUCGGCAACCCCCACGUGUCCGGCGGCCUGAAGGAGGGCAUC |
| | | GAGACCCUGCUGAUGACCCAGCGGCCCGCCCUGUCCGGCGACGCCGUGCAGAAGAUGCUGGGCCACUGCACCUUCGAGCCCGCCGAGCCCA |
| | | AGGCCGCCAAGAACACCUACACCGCCGAGCGGUUCAUCUGGCUGACCAAGCUGAACAACCUGCGGAUCCUGGAGCAGGGCUCCGAGCGGCC |
| | | CCUGACCGACACCGAGCGGGCCACCCUGAUGGACGAGCCCUACCGGAAGUCCAAGCUGACCUACGCCCAGGCCCGGAAGCUGCUGGGCCUG |
| | | GAGGACACCGCCUUCUUCAAGGGCCUGCGGUACGGCAAGGACAACGCCGAGGCCUCCACCCUGAUGGAGAUGAAGGCCUACCACGCCAUCU |
| | | CCCGGGCCCUGGAGAAGGAGGGCCUGAAGGACAAGAAGUCCCCCCUGAACCUGUCCUCCGAGCUGCAGGACGAGAUCGGCACCGCCUUCUC |
| | | CCUGUUCAAGACCGACGAGGACAUCACCGGCCGGCUGAAGGACCGGGUGCAGCCCGAGAUCCUGGAGGCCCUGCUGAAGCACAUCUCCUUC |
| | | GACAAGUUCGUGCAGAUCUCCCUGAAGGCCCUGCGGCGGAUCGUGCCCCUGAUGGAGCAGGGCAAGCGGUACGACGAGGCCUGCGCCGAGA |
| | | UCUACGGCGACCACUACGGCAAGAAGAACACCGAGGAGAAGAUCUACCUGCCCCCCAUCCCCGCCGACGAGAUCCGGAACCCCGUGGUGCU |
| | | GCGGGCCCUGUCCCAGGCCCGGAAGGUGAUCAACGGCGUGGUGCGGCGGUACGGCUCCCCCGCCCGGAUCCACAUCGAGACCGCCCGGGAG |
| | | GUGGGCAAGUCCUUCAAGGACCGGAAGGAGAUCGAGAAGCGGCAGGAGGAGAACCGGAAGGACCGGGAGAAGGCCGCCGCCAAGUUCCGGG |
| | | AGUACUUCCCCAACUUCGUGGGCGAGCCCAAGUCCAAGGACAUCCUGAAGCUGCGGCUGUACGAGCAGCAGCACGGCAAGUGCCUGUACUC |
| | | CGGCAAGGAGAUCAACCUGGUGCGGCUGAACGAGAAGGGCUACGUGGAGAUCGACCACGCCCUGCCCUUCUCCCGGACCUGGGACGACUCC |
| | | UUCAACAACAAGGUGCUGGUGCUGGGCUCCGAGAACCAGAACAAGGGCAACCAGACCCCCUACGAGUACUUCAACGGCAAGGACAACUCCC |
| | | GGGAGUGGCAGGAGUUCAAGGCCCGGGUGGAGACCUCCCGGUUCCCCCGGUCCAAGAAGCAGCGGAUCCUGCUGCAGAAGUUCGACGAGGA |
| | | CGGCUUCAAGGAGUGCAACCUGAACGACACCCGGUACGUGAACCGCUUCCUGUGCCAGUUCGUGGCCGACCACAUCCUGCUGACCGGCAAG |
| | | GGCAAGCGGCGGGUGUUCGCCUCCAACGGCCAGAUCACCAACCUGCUGCGGGGCUUCUGGGGCCUGCGGAAGGUGCGGGCCGAGAACGACC |
| | | GGCACCACGCCCUGGACGCCGUGGUGGUGGCCUGCUCCACCGUGGCCAUGCAGCAGAAGAUCACCCGGUUCGUGCGGUACAAGGAGAUGAA |
| | | CGCCUUCGACGGCAAGACCAUCGACAAGGAGACCGGCAAGGUGCUGCACCAGAAGACCCACUUCCCCCAGCCCUGGGAGUUCUUCGCCCAG |
| | | GAGGUGAUGAUCCGGGUGUUCGGCAAGCCCGACGGCAAGCCCGAGUUCGAGGAGGCCGACACCCCCGAGAAGCUGCGGACCCUGCUGGCCG |
| | | AGAAGCUGUCCUCCCGGCCCGAGGCCGUGCACGAGUACGUGACCCCCCUGUUCGUGUCCCGGGCCCCCAACCGGAAGAUGUCCGGCGCCCA |
| | | CAAGGACACCCUGCGGUCCGCCAAGCGGUUCGUGAAGCACAACGAGAAGAUCUCCGUGAAGCGGGUGUGGCUGACCGAGAUCAAGCUGGCC |
| | | GACCUGGAGAACAUGGUGAACUACAAGAACGGCCGGGAGAUCGAGCUGUACGAGGCCCUGAAGGCCCGGCUGGAGGCCUACGGCGGCAACG |
| | | CCAAGCAGGCCUUCGACCCCAAGGACAACCCCUUCUACAAGAAGGGCGGCCAGCUGGUGAAGGCCGUGCGGGUGGAGAAGACCCAGGAGUC |
| | | CGGCGUGCUGCUGAACAAGAAGAACGCCUACACCAUCGCCGACAACGGCGACAUGGUGCGGGUGGACGUGUUCUGCAAGGUGGACAAGAAG |
| | | GGCAAGAACCAGUACUUCAUCGUGCCCAUCUACGCCUGGCAGGUGGCCGAGAACAUCCUGCCCGACAUCGACUGCAAGGGCUACCGGAUCG |
| | | ACGACUCCUACACCUUCUGCUUCUCCCUGCACAAGUACGACCUGAUCGCCUUCCAGAAGGACGAGAAGUCCAAGGUGGAGUUCGCCUACUA |
| | | CAUCAACUGCGACUCCUCCAACGGCCGGUUCUACCUGGCCUGGCACGACAAGGGCUCCAAGGAGCAGCAGUUCCGGAUCUCCACCCAGAAC |
| | | CUGGUGCUGAUCCAGAAGUACCAGGUGAACGAGCUGGGCAAGGAGAUCCGGCCCUGCCGGCUGAAGAAGCGGCCCCCCGUGCGGUCCGGAA |
| | | AGCGGACCGCCGACGGCUCCGGAGGAGGAAGCCCGAAGAAGAAGAGAAAGGUCAGCGAAAGCGCAACACCGGAAAGCGUCAGCGGAUGGAG |
| | | ACUGUUCAAGAAGAUCAGCUAGCUAGCACCAGCCUCAAGAACACCCGAAUGGAGUCUCUAAGCUACAUAAUACCAACUUACACUUUACAAA |
| | | AUGUUGUCCCCCAAAAUGUAGCCAUUCGUAUCUGCUCCUAAUAAAAAGAAAGUUUCUUCACAUUCUCUCGAGAAAAAAAAAAAAUGGAAAA |
| | | AAAAAAAACGGAAAAAAAAAAAAGGUAAAAAAAAAAAAUAUAAAAAAAAAAAACAUAAAAAAAAAAAACGAAAAAAAAAAAACGUAAAAAA |
| | | AAAAAACUCAAAAAAAAAAAAGAUAAAAAAAAAAAACCUAAAAAAAAAAAAUGUAAAAAAAAAAAAGGGAAAAAAAAAAAACGCAAAAAAA |
| | | AAAAACACAAAAAAAAAAAAUGCAAAAAAAAAAAAUCGAAAAAAAAAAAAUCUAAAAAAAAAAAACGAAAAAAAAAAAACCCAAAAAAAAA |
| | | AAAGACAAAAAAAAAAAAUAGAAAAAAAAAAAAGUUAAAAAAAAAAAACUGAAAAAAAAAAAAUUUAAAAAAAAAAAAUCUAG |
| 42 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion | CACCCGGCUGAAGCGGACCGCCCGGCGGCGGUACACCCGGCGGAAGAACCGGAUCUGCUACCUGCAGGAGAUCUUCUCCAACGAGAUGGCC |
| | with | AAGGUGGACGACUCCUUCUUCCACCGGCUGGAGGAGUCCUUCCUGGUGGAGGAGGACAAGAAGCACGAGCGGCACCCCAUCUUCGGCAACA |
| | HiBit | UCGUGGACGAGGUGGCCUACCACGAGAAGUACCCCACCAUCUACCACCUGCGGAAGAAGCUGGUGGACUCCACCGACAAGGCCGACCUGCG |
| | tag | GCUGAUCUACCUGGCCCUGGCCCACAUGAUCAAGUUCCGGGGCCACUUCCUGAUCGAGGGCGACCUGAACCCCGACAACUCCGACGUGGAC |
| | (mRNA | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | M)-ORF | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | sequence | CCUGUCCCUGGGCCUGACCCCCAACUUCAAGUCCAACUUCGACCUGGCCGAGGACGCCAAGCUGCAGCUGUCCAAGGACACCUACGACGAC |
| | | GACCUGGACAACCUGCUGGCCCAGAUCGGCGACCAGUACGCCGACCUGUUCCUGGCCGCCAAGAACCUGUCCGACGCCAUCCUGCUGUCCG |
| | | ACAUCCUGCGGGUGAACACCGAGAUCACCAAGGCCCCCCUGUCCGCCUCCAUGAUCAAGCGGUACGACGAGCACCACCAGGACCUGACCCU |
| | | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCGCCGGCACCUCCACCGAGUCCGAGGG |
| | | CUCCGCCGGCACCUCCGAGUCCGCCACCGAGUCCGGCGGCACCUCCACCGAGUCCGAGGGCUCCUCCUCCGGUGCCGCCUUCAAGCCCAAC |
| | | CCCAUCAACUACAUCCUGGGCCUGGACAUCGGCAUCGCCUCCGUGGGCUGGGCCAUGGUGGAGAUCGACGAGGAGGAGAACCCCAUCCGGC |
| | | UGAUCGACCUGGGCGUGCGGGUGUUCGAGCGGGCCGAGGUGCCCAAGACCGGCGACUCCCUGGCCAUGGCCCGGCGGCUGGCCCGGUCCGU |
| | | GCGGCGGCUGACCCGGCGGCGGGCCCACCGGCUGCUGCGGGCCCGGCGGCUGCUGAAGCGGGAGGGCGUGCUGCAGGCCGCCGACUUCGAC |
| | | GAGAACGGCCUGAUCAAGUCCCUGCCCAACACCCCCUGGCAGCUGCGGGCCGCCGCCCUGGACCGGAAGCUGACCCCCCUGGAGUGGUCCG |
| | | CCGUGCUGCUGCACCUGAUCAAGCACCGGGGCUACCUGUCCCAGCGGAAGAACGAGGGCGAGACCGCCGACAAGGAGCUGGGCGCCCUGCU |
| | | GAAGGGCGUGGCCAACAACGCCCACGCCCUGCAGACCGGCGACUUCCGGACCCCCGCCGAGCUGGCCCUGAACAAGUUCGAGAAGGAGUCC |
| | | GGCCACAUCCGGAACCAGCGGGGCGACUACUCCCACACCUUCUCCCGGAAGGACCUGCAGGCCGAGCUGAUCCUGCUGUUCGAGAAGCAGA |
| | | AGGAGUUCGGCAACCCCCACGUGUCCGGCGGCCUGAAGGAGGGCAUCGAGACCCUGCUGAUGACCCAGCGGCCCGCCCUGUCCGGCGACGC |
| | | CGUGCAGAAGAUGCUGGGCCACUGCACCUUCGAGCCCGCCGAGCCCAAGGCCGCCAAGAACACCUACACCGCCGAGCGGUUCAUCUGGCUG |
| | | ACCAAGCUGAACAACCUGCGGAUCCUGGAGCAGGGCUCCGAGCGGCCCCUGACCGACACCGAGCGGGCCACCCUGAUGGACGAGCCCUACC |
| | | GGAAGUCCAAGCUGACCUACGCCCAGGCCCGGAAGCUGCUGGGCCUGGAGGACACCGCCUUCUUCAAGGGCCUGCGGUACGGCAAGGACAA |
| | | CGCCGAGGCCUCCACCCUGAUGGAGAUGAAGGCCUACCACGCCAUCUCCCGGGCCCUGGAGAAGGAGGGCCUGAAGGACAAGAAGUCCCCC |
| | | CUGAACCUGUCCUCCGAGCUGCAGGACGAGAUCGGCACCGCCUUCUCCCUGUUCAAGACCGACGAGGACAUCACCGGCCGGCUGAAGGACC |
| | | GGGUGCAGCCCGAGAUCCUGGAGGCCCUGCUGAAGCACAUCUCCUUCGACAAGUUCGUGCAGAUCUCCCUGAAGGCCCUGCGGCGGAUCGU |
| | | GCCCCUGAUGGAGCAGGGCAAGCGGUACGACGAGGCCUGCGCCGAGAUCUACGGCGACCACUACGGCAAGAAGAACACCGAGGAGAAGAUC |
| | | UACCUGCCCCCCAUCCCCGCCGACGAGAUCCGGAACCCCGUGGUGCUGCGGGCCCUGUCCCAGGCCCGGAAGGUGAUCAACGGCGUGGUGC |
| | | GGCGGUACGGCUCCCCCGCCCGGAUCCACAUCGAGACCGCCCGGGAGGUGGGCAAGUCCUUCAAGGACCGGAAGGAGAUCGAGAAGCGGCA |
| | | GGAGGAGAACCGGAAGGACCGGGAGAAGGCCGCCGCCAAGUUCCGGGAGUACUUCCCCAACUUCGUGGGCGAGCCCAAGUCCAAGGACAUC |
| | | CUGAAGCUGCGGCUGUACGAGCAGCAGCACGGCAAGUGCCUGUACUCCGGCAAGGAGAUCAACCUGGUGCGGCUGAACGAGAAGGGCUACG |
| | | UGGAGAUCGACCACGCCCUGCCCUUCUCCCGGACCUGGGACGACUCCUUCAACAACAAGGUGCUGGUGCUGGGCUCCGAGAACCAGAACAA |
| | | GGGCAACCAGACCCCCUACGAGUACUUCAACGGCAAGGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCAAGGAGUGCAACCUGAACGACACCCGGUACGUGAACC |
| | | GCUUCCUGUGCCAGUUCGUGGCCGACCACAUCCUGCUGACCGGCAAGGGCAAGCGGCGGGUGUUCGCCUCCAACGGCCAGAUCACCAACCU |
| | | GCUGCGGGGCUUCUGGGGCCUGCGGAAGGUGCGGGCCGAGAACGACCGGCACCACGCCCUGGACGCCGUGGUGGUGGCCUGCUCCACCGUG |
| | | GCCAUGCAGCAGAAGAUCACCCGGUUCGUGCGGUACAAGGAGAUGAACGCCUUCGACGGCAAGACCAUCGACAAGGAGACCGGCAAGGUGC |
| | | UGCACCAGAAGACCCACUUCCCCCAGCCCUGGGAGUUCUUCGCCCAGGAGGUGAUGAUCCGGGUGUUCGGCAAGCCCGACGGCAAGCCCGA |
| | | GUUCGAGGAGGCCGACACCCCCGAGAAGCUGCGGACCCUGCUGGCCGAGAAGCUGUCCUCCCGGCCCGAGGCCGUGCACGAGUACGUGACC |
| | | CCCCUGUUCGUGUCCCGGGCCCCCAACCGGAAGAUGUCCGGCGCCCACAAGGACACCCUGCGGUCCGCCAAGCGGUUCGUGAAGCACAACG |
| | | AGAAGAUCUCCGUGAAGCGGGUGUGGCUGACCGAGAUCAAGCUGGCCGACCUGGAGAACAUGGUGAACUACAAGAACGGCCGGGAGAUCGA |
| | | GCUGUACGAGGCCCUGAAGGCCCGGCUGGAGGCCUACGGCGGCAACGCCAAGCAGGCCUUCGACCCCAAGGACAACCCCUUCUACAAGAAG |
| | | GGCGGCCAGCUGGUGAAGGCCGUGCGGGUGGAGAAGACCCAGGAGUCCGGCGUGCUGCUGAACAAGAAGAACGCCUACACCAUCGCCGACA |
| | | ACGGCGACAUGGUGCGGGUGGACGUGUUCUGCAAGGUGGACAAGAAGGGCAAGAACCAGUACUUCAUCGUGCCCAUCUACGCCUGGCAGGU |
| | | GGCCGAGAACAUCCUGCCCGACAUCGACUGCAAGGGCUACCGGAUCGACGACUCCUACACCUUCUGCUUCUCCCUGCACAAGUACGACCUG |
| | | AUCGCCUUCCAGAAGGACGAGAAGUCCAAGGUGGAGUUCGCCUACUACAUCAACUGCGACUCCUCCAACGGCCGGUUCUACCUGGCCUGGC |
| | | ACGACAAGGGCUCCAAGGAGCAGCAGUUCCGGAUCUCCACCCAGAACCUGGUGCUGAUCCAGAAGUACCAGGUGAACGAGCUGGGCAAGGA |
| | | GAUCCGGCCCUGCCGGCUGAAGAAGCGGCCCCCCGUGCGGUCCGGAAAGCGGACCGCCGACGGCUCCGGAGGAGGAAGCCCGAAGAAGAAG |
| | | AGAAAGGUCAGCGAAAGCGCAACACCGGAAAGCGUCAGCGGAUGGAGACUGUUCAAGAAGAUCAGCUAG |
| 43 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion | KLFIQLVQTYNQLFEENPINASGVDAKAILSARLSKSRRLENLIAQLPGEKKNGLFGNLIALSLGLTPNFKSNFDLAEDAKLQLSKDTYDD |
| | with | DLDNLLAQIGDQYADLFLAAKNLSDAILLSDILRVNTEITKAPLSASMIKRYDEHHQDLTLLKALVRQQLPEKYKEIFFDQSKNGYAGYID |
| | HiBit | GGASQEEFYKFIKPILEKMDGTEELLVKLNREDLLRKQRTFDNGSIPHQIHLGELHAILRRQEDFYPFLKDNREKIEKILTFRIPYYVGPL |
| | tag | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | (mRNA | AIVDLLFKTNRKVTVKQLKEDYFKKIECFDSVEISGVEDRFNASLGTYHDLLKIIKDKDFLDNEENEDILEDIVLTLTLFEDREMIEERLK |
| | M)- | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | amino | GSPAIKKGILQTVKVVDELVKVMGRHKPENIVIEMARENQTTQKGQKNSRERMKRIEEGIKELGSQILKEHPVENTQLQNEKLYLYYLQNG |
| | acid | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | sequence | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | PINYILGLDIGIASVGWAMVEIDEEENPIRLIDLGVRVFERAEVPKTGDSLAMARRLARSVRRLTRRRAHRLLRARRLLKREGVLQAADFD |
| | | ENGLIKSLPNTPWQLRAAALDRKLTPLEWSAVLLHLIKHRGYLSQRKNEGETADKELGALLKGVANNAHALQTGDFRTPAELALNKFEKES |
| | | GHIRNQRGDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLVRLNEKGYVEIDHALPFSRTWDDSFNNKVLVLGSENQNKGNQTPYEYFNGKDNSREWQEFKARVETSRF |
| | | PRSKKQRILLQKFDEDGFKECNLNDTRYVNRFLCQFVADHILLTGKGKRRVFASNGQITNLLRGFWGLRKVRAENDRHHALDAVVVACSTV |
| | | AMQQKITRFVRYKEMNAFDGKTIDKETGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEALKARLEAYGGNAKQAFDPKDNPFYKK |
| | | GGQLVKAVRVEKTQESGVLLNKKNAYTIADNGDMVRVDVFCKVDKKGKNQYFIVPIYAWQVAENILPDIDCKGYRIDDSYTFCFSLHKYDL |
| | | IAFQKDEKSKVEFAYYINCDSSNGRFYLAWHDKGSKEQQFRISTQNLVLIQKYQVNELGKEIRPCRLKKRPPVRSGKRTADGSGGGSPKKK |
| | | RKVSESATPESVSGWRLFKKIS* |
| 44 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion | CACCCGGCUGAAGCGGACCGCCCGGCGGCGGUACACCCGGCGGAAGAACCGGAUCUGCUACCUGCAGGAGAUCUUCUCCAACGAGAUGGCC |
| | (mRNA | AAGGUGGACGACUCCUUCUUCCACCGGCUGGAGGAGUCCUUCCUGGUGGAGGAGGACAAGAAGCACGAGCGGCACCCCAUCUUCGGCAACA |
| | M)-ORF | UCGUGGACGAGGUGGCCUACCACGAGAAGUACCCCACCAUCUACCACCUGCGGAAGAAGCUGGUGGACUCCACCGACAAGGCCGACCUGCG |
| | sequence | GCUGAUCUACCUGGCCCUGGCCCACAUGAUCAAGUUCCGGGGCCACUUCCUGAUCGAGGGCGACCUGAACCCCGACAACUCCGACGUGGAC |
| | | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | | CCUGUCCCUGGGCCUGACCCCCAACUUCAAGUCCAACUUCGACCUGGCCGAGGACGCCAAGCUGCAGCUGUCCAAGGACACCUACGACGAC |
| | | GACCUGGACAACCUGCUGGCCCAGAUCGGCGACCAGUACGCCGACCUGUUCCUGGCCGCCAAGAACCUGUCCGACGCCAUCCUGCUGUCCG |
| | | ACAUCCUGCGGGUGAACACCGAGAUCACCAAGGCCCCCCUGUCCGCCUCCAUGAUCAAGCGGUACGACGAGCACCACCAGGACCUGACCCU |
| | | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCGCCGGCACCUCCACCGAGUCCGAGGG |
| | | CUCCGCCGGCACCUCCGAGUCCGCCACCGAGUCCGGCGGCACCUCCACCGAGUCCGAGGGCUCCUCCUCCGGUGCCGCCUUCAAGCCCAAC |
| | | CCCAUCAACUACAUCCUGGGCCUGGACAUCGGCAUCGCCUCCGUGGGCUGGGCCAUGGUGGAGAUCGACGAGGAGGAGAACCCCAUCCGGC |
| | | UGAUCGACCUGGGCGUGCGGGUGUUCGAGCGGGCCGAGGUGCCCAAGACCGGCGACUCCCUGGCCAUGGCCCGGCGGCUGGCCCGGUCCGU |
| | | GCGGCGGCUGACCCGGCGGCGGGCCCACCGGCUGCUGCGGGCCCGGCGGCUGCUGAAGCGGGAGGGCGUGCUGCAGGCCGCCGACUUCGAC |
| | | GAGAACGGCCUGAUCAAGUCCCUGCCCAACACCCCCUGGCAGCUGCGGGCCGCCGCCCUGGACCGGAAGCUGACCCCCCUGGAGUGGUCCG |
| | | CCGUGCUGCUGCACCUGAUCAAGCACCGGGGCUACCUGUCCCAGCGGAAGAACGAGGGCGAGACCGCCGACAAGGAGCUGGGCGCCCUGCU |
| | | GAAGGGCGUGGCCAACAACGCCCACGCCCUGCAGACCGGCGACUUCCGGACCCCCGCCGAGCUGGCCCUGAACAAGUUCGAGAAGGAGUCC |
| | | GGCCACAUCCGGAACCAGCGGGGCGACUACUCCCACACCUUCUCCCGGAAGGACCUGCAGGCCGAGCUGAUCCUGCUGUUCGAGAAGCAGA |
| | | AGGAGUUCGGCAACCCCCACGUGUCCGGCGGCCUGAAGGAGGGCAUCGAGACCCUGCUGAUGACCCAGCGGCCCGCCCUGUCCGGCGACGC |
| | | CGUGCAGAAGAUGCUGGGCCACUGCACCUUCGAGCCCGCCGAGCCCAAGGCCGCCAAGAACACCUACACCGCCGAGCGGUUCAUCUGGCUG |
| | | ACCAAGCUGAACAACCUGCGGAUCCUGGAGCAGGGCUCCGAGCGGCCCCUGACCGACACCGAGCGGGCCACCCUGAUGGACGAGCCCUACC |
| | | GGAAGUCCAAGCUGACCUACGCCCAGGCCCGGAAGCUGCUGGGCCUGGAGGACACCGCCUUCUUCAAGGGCCUGCGGUACGGCAAGGACAA |
| | | CGCCGAGGCCUCCACCCUGAUGGAGAUGAAGGCCUACCACGCCAUCUCCCGGGCCCUGGAGAAGGAGGGCCUGAAGGACAAGAAGUCCCCC |
| | | CUGAACCUGUCCUCCGAGCUGCAGGACGAGAUCGGCACCGCCUUCUCCCUGUUCAAGACCGACGAGGACAUCACCGGCCGGCUGAAGGACC |
| | | GGGUGCAGCCCGAGAUCCUGGAGGCCCUGCUGAAGCACAUCUCCUUCGACAAGUUCGUGCAGAUCUCCCUGAAGGCCCUGCGGCGGAUCGU |
| | | GCCCCUGAUGGAGCAGGGCAAGCGGUACGACGAGGCCUGCGCCGAGAUCUACGGCGACCACUACGGCAAGAAGAACACCGAGGAGAAGAUC |
| | | UACCUGCCCCCCAUCCCCGCCGACGAGAUCCGGAACCCCGUGGUGCUGCGGGCCCUGUCCCAGGCCCGGAAGGUGAUCAACGGCGUGGUGC |
| | | GGCGGUACGGCUCCCCCGCCCGGAUCCACAUCGAGACCGCCCGGGAGGUGGGCAAGUCCUUCAAGGACCGGAAGGAGAUCGAGAAGCGGCA |
| | | GGAGGAGAACCGGAAGGACCGGGAGAAGGCCGCCGCCAAGUUCCGGGAGUACUUCCCCAACUUCGUGGGCGAGCCCAAGUCCAAGGACAUC |
| | | CUGAAGCUGCGGCUGUACGAGCAGCAGCACGGCAAGUGCCUGUACUCCGGCAAGGAGAUCAACCUGGUGCGGCUGAACGAGAAGGGCUACG |
| | | UGGAGAUCGACCACGCCCUGCCCUUCUCCCGGACCUGGGACGACUCCUUCAACAACAAGGUGCUGGUGCUGGGCUCCGAGAACCAGAACAA |
| | | GGGCAACCAGACCCCCUACGAGUACUUCAACGGCAAGGACAACUCCCGGGAGUGGCAGGAGUUCAAGGCCCGGGUGGAGACCUCCCGGUUC |
| | | CCCCGGUCCAAGAAGCAGCGGAUCCUGCUGCAGAAGUUCGACGAGGACGGCUUCAAGGAGUGCAACCUGAACGACACCCGGUACGUGAACC |
| | | GCUUCCUGUGCCAGUUCGUGGCCGACCACAUCCUGCUGACCGGCAAGGGCAAGCGGCGGGUGUUCGCCUCCAACGGCCAGAUCACCAACCU |
| | | GCUGCGGGGCUUCUGGGGCCUGCGGAAGGUGCGGGCCGAGAACGACCGGCACCACGCCCUGGACGCCGUGGUGGUGGCCUGCUCCACCGUG |
| | | GCCAUGCAGCAGAAGAUCACCCGGUUCGUGCGGUACAAGGAGAUGAACGCCUUCGACGGCAAGACCAUCGACAAGGAGACCGGCAAGGUGC |
| | | UGCACCAGAAGACCCACUUCCCCCAGCCCUGGGAGUUCUUCGCCCAGGAGGUGAUGAUCCGGGUGUUCGGCAAGCCCGACGGCAAGCCCGA |
| | | GUUCGAGGAGGCCGACACCCCCGAGAAGCUGCGGACCCUGCUGGCCGAGAAGCUGUCCUCCCGGCCCGAGGCCGUGCACGAGUACGUGACC |
| | | CCCCUGUUCGUGUCCCGGGCCCCCAACCGGAAGAUGUCCGGCGCCCACAAGGACACCCUGCGGUCCGCCAAGCGGUUCGUGAAGCACAACG |
| | | AGAAGAUCUCCGUGAAGCGGGUGUGGCUGACCGAGAUCAAGCUGGCCGACCUGGAGAACAUGGUGAACUACAAGAACGGCCGGGAGAUCGA |
| | | GCUGUACGAGGCCCUGAAGGCCCGGCUGGAGGCCUACGGCGGCAACGCCAAGCAGGCCUUCGACCCCAAGGACAACCCCUUCUACAAGAAG |
| | | GGCGGCCAGCUGGUGAAGGCCGUGCGGGUGGAGAAGACCCAGGAGUCCGGCGUGCUGCUGAACAAGAAGAACGCCUACACCAUCGCCGACA |
| | | ACGGCGACAUGGUGCGGGUGGACGUGUUCUGCAAGGUGGACAAGAAGGGCAAGAACCAGUACUUCAUCGUGCCCAUCUACGCCUGGCAGGU |
| | | GGCCGAGAACAUCCUGCCCGACAUCGACUGCAAGGGCUACCGGAUCGACGACUCCUACACCUUCUGCUUCUCCCUGCACAAGUACGACCUG |
| | | AUCGCCUUCCAGAAGGACGAGAAGUCCAAGGUGGAGUUCGCCUACUACAUCAACUGCGACUCCUCCAACGGCCGGUUCUACCUGGCCUGGC |
| | | ACGACAAGGGCUCCAAGGAGCAGCAGUUCCGGAUCUCCACCCAGAACCUGGUGCUGAUCCAGAAGUACCAGGUGAACGAGCUGGGCAAGGA |
| | | GAUCCGGCCCUGCCGGCUGAAGAAGCGGCCCCCCGUGCGGUCCGGAAAGCGGACCGCCGACGGCUCCGGAGGAGGAAGCCCGAAGAAGAAG |
| | | AGAAAGGUCUAG |
| 45 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion | KLFIQLVQTYNQLFEENPINASGVDAKAILSARLSKSRRLENLIAQLPGEKKNGLFGNLIALSLGLTPNFKSNFDLAEDAKLQLSKDTYDD |
| | (mRNA | DLDNLLAQIGDQYADLFLAAKNLSDAILLSDILRVNTEITKAPLSASMIKRYDEHHQDLTLLKALVRQQLPEKYKEIFFDQSKNGYAGYID |
| | M)- | GGASQEEFYKFIKPILEKMDGTEELLVKLNREDLLRKQRTFDNGSIPHQIHLGELHAILRRQEDFYPFLKDNREKIEKILTFRIPYYVGPL |
| | amino | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | acid | AIVDLLFKTNRKVTVKQLKEDYFKKIECFDSVEISGVEDRFNASLGTYHDLLKIIKDKDFLDNEENEDILEDIVLTLTLFEDREMIEERLK |
| | sequence | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | PINYILGLDIGIASVGWAMVEIDEEENPIRLIDLGVRVFERAEVPKTGDSLAMARRLARSVRRLTRRRAHRLLRARRLLKREGVLQAADFD |
| | | ENGLIKSLPNTPWQLRAAALDRKLTPLEWSAVLLHLIKHRGYLSQRKNEGETADKELGALLKGVANNAHALQTGDFRTPAELALNKFEKES |
| | | GHIRNQRGDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLVRLNEKGYVEIDHALPFSRTWDDSFNNKVLVLGSENQNKGNQTPYEYFNGKDNSREWQEFKARVETSRF |
| | | PRSKKQRILLQKFDEDGFKECNLNDTRYVNRFLCQFVADHILLTGKGKRRVFASNGQITNLLRGFWGLRKVRAENDRHHALDAVVVACSTV |
| | | AMQQKITRFVRYKEMNAFDGKTIDKETGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEALKARLEAYGGNAKQAFDPKDNPFYKK |
| | | GGQLVKAVRVEKTQESGVLLNKKNAYTIADNGDMVRVDVFCKVDKKGKNQYFIVPIYAWQVAENILPDIDCKGYRIDDSYTFCFSLHKYDL |
| | | IAFQKDEKSKVEFAYYINCDSSNGRFYLAWHDKGSKEQQFRISTQNLVLIQKYQVNELGKEIRPCRLKKRPPVRSGKRTADGSGGGSPKKK |
| | | RKV* |
| 46 | R1333 | auggacaagaaguacuccaucggccuggacaucggcaccaacuccgugggcugggccgugaucaccgacgaguacaaggugcccuccaaga |
| | WT | aguucaaggugcugggcaacaccgaccggcacuccaucaagaagaaccugaucggcgcccugcuguucgacuccggcgagaccgccgaggc |
| | Cas9- | cacccggcugaagcggaccgcccggcggcgguacacccggcggaagaaccggaucugcuaccugcaggagaucuucuccaacgagauggcc |
| | Cas9 | aagguggacgacuccuucuuccaccggcuggaggaguccuuccugguggaggaggacaagaagcacgagcggcaccccaucuucggcaaca |
| | fusion | ucguggacgagguggccuaccacgagaaguaccccaccaucuaccaccugcggaagaagcugguggacuccaccgacaaggccgaccugcg |
| | PA1-4 | gcugaucuaccuggcccuggcccacaugaucaaguuccgggcgccacuuccugaucgaggggaccugaaccccgacaacuccgacguggac |
| | (mRNA | aagcuguucauccagcuggugcagaccuacaaccagcuguucgaggagaaccccaucaacgccuccggcguggacgccaaggccauccugu |
| | N)- | ccgcccggcuguccaagucccggcggcuggagaaccugaucgcccagcugcccggcgagaagaagaacggccuguucggcaaccugaucgc |
| | ORF | ccugucccugggccugacccccaacuucaaguccaacuucgaccuggccgaggacgccaagcugcagcuguccaaggacaccuacgacgac |
| | sequence | gaccuggacaaccugcuggcccagaucggcgaccaguacgccgaccuguuccuggccgccaagaaccuguccgacgccauccugcuguccg |
| | (Example | acauccugcgggugaacaccgagaucaccaaggccccccuguccgccuccaugaucaagcgguacgacgagcaccaccaggaccugacccu |
| | 4) | gcugaaggcccuggugcggcagcagcugcccgagaaguacaaggagaucuucuucgaccaguccaagaacggcuacgccggcuacaucgac |
| | | ggcggcgccucccaggaggaguucuacaaguucaucaagcccauccuggagaagauggacggcaccgaggagcugcuggugaagcugaacc |
| | | gggaggaccugcugcggaagcagcggaccuucgacaacggcuccaucccccaccagauccaccugggcgagcugcacgccauccugcggcg |
| | | gcaggaggacuucuaccccuuccugaaggacaaccgggagaagaucgagaagauccugaccuuccggauccccuacuacgugggcccccug |
| | | gcccggggcaacucccgguucgccuggaugacccggaaguccgaggagaccaucacccccuggaacuucgaggaggugguggacaagggcg |
| | | ccuccgcccaguccuucaucgagcggaugaccaacuucgacaagaaccugcccaacgagaaggugcugcccaagcacucccugcuguacga |
| | | guacuucaccguguacaacgagcugaccaaggugaaguacgugaccgagggcaugcggaagcccgccuuccuguccggcgagcagaagaag |
| | | gccaucguggaccugcuguucaagaccaaccggaaggugaccgugaagcagcugaaggaggacuacuucaagaagaucgagugcuucgacu |
| | | ccguggagaucuccggcguggaggaccgguucaacgccucccugggcaccuaccacgaccugcugaagaucaucaaggacaaggacuuccu |
| | | ggacaacgaggagaacgaggacauccuggaggacaucgugcugacccugacccuguucgaggaccgggagaugaucgaggagcggcugaag |
| | | accuacgcccaccuguucgacgacaaggugaugaagcagcugaagcggcggcgguacaccggcuggggccggcugucccggaagcugauca |
| | | acggcauccgggacaagcaguccggcaagaccauccuggacuuccugaaguccgacggcuucgccaaccggaacuucaugcagcugaucca |
| | | cgacgacucccugaccuucaaggaggacauccagaaggcccagguguccggccagggcgacucccugcacgagcacaucgccaaccuggcc |
| | | ggcucccccgccaucaagaagggcauccugcagaccgugaaggugguggacgagcuggugaaggugaugggccggcacaagcccgagaaca |
| | | ucgugaucgagauggcccgggagaaccagaccacccagaagggccagaagaacucccgggagcggaugaagcggaucgaggagggcaucaa |
| | | ggagcugggcucccagauccugaaggagcaccccguggagaacacccagcugcagaacgagaagcuguaccuguacuaccugcagaacggc |
| | | cgggacauguacguggaccaggagcuggacaucaaccggcuguccgacuacgacguggaccacaucgugccccaguccuuccugaaggacg |
| | | acuccaucgacaacaaggugcugacccgguccgacaagaaccggggcaaguccgacaacgugcccuccgaggagguggugaagaagaugaa |
| | | gaacuacuggcggcagcugcugaacgccaagcugaucacccagcggaaguucgacaaccugaccaaggccgagcggcggggccuguccgag |
| | | cuggacaaggccggcuucaucaagcggcagcugguggagacccggcagaucaccaagcacguggcccagauccuggacucccggaugaaca |
| | | ccaaguacgacgagaacgacaagcugauccgggaggugaaggugaucacccugaaguccaagcugguguccgacuuccggaaggacuucca |
| | | guucuacaaggugcgggagaucaacaacuaccaccacgcccacgacgccuaccugaacgccguggugggcaccgcccugaucaagaaguac |
| | | cccaagcuggaguccgaguucguguacggcgacuacaagguguacgacgugcggaagaugaucgccaaguccgagcaggagaucggcaagg |
| | | ccaccgccaaguacuucuucuacuccaacaucaugaacuucuucaagaccgagaucacccuggccaacggcgagauccggaagcggccccu |
| | | gaucgagaccaacggcgagaccggcgagaucgugugggacaagggccgggacuucgccaccgugcggaaggugcuguccaugccccaggug |
| | | aacaucgugaagaagaccgaggugcagaccggcggcuucuccaaggaguccauccugcccaagcggaacuccgacaagcugaucgcccgga |
| | | agaaggacugggaccccaagaaguacggcggcuucgacucccccaccguggccuacuccgugcuggugguggccaagguggagaagggcaa |
| | | guccaagaagcugaaguccgugaaggagcugcugggcaucaccaucauggagcgguccuccuucgagaagaaccccaucgacuuccuggag |
| | | gccaagggcuacaaggaggugaagaaggaccugaucaucaagcugcccaaguacucccuguucgagcuggagaacggccggaagcggaugc |
| | | uggccuccgccggcgagcugcagaagggcaacgagcuggcccugcccuccaaguacgugaacuuccuguaccuggccucccacuacgagaa |
| | | gcugaagggcucccccgaggacaacgagcagaagcagcuguucguggagcagcacaagcacuaccuggacgagaucaucgagcagaucucc |
| | | gaguucuccaagcgggugauccuggccgacgccaaccuggacaaggugcuguccgccuacaacaagcaccgggacaagcccauccgggagc |
| | | aggccgagaacaucauccaccuguucacccugaccaaccugggcgcccccgccgccuucaaguacuucgacaccaccaucgacCGGaagcg |
| | | guacaccuccaccaaggaggugcuggacgccacccugauccaccaguccaucaccggccuguacgagacccggaucgaccugucccagcug |
| | | ggcggcgacGGCgccCCCgagUCCgccACCgagUCCggcGGCaccUCCaccGAGuccGAGggcUCCgccGGCaccUCCaccGAGuccGAGg |
| | | gcuccGCCggcUCCgccGGCuccaccUCCaccGAGgagGGCaccUCCaccGAGuccGAGggcuccGCCggcACCuccACCgaguccgagGG |
| | | CuccGCCggcACCuccGAGuccgccACCgagUCCggcGGCaccUCCaccGAGuccGAGggCUCCuccUCCggugccgccuucaagcccaac |
| | | cccaucaacuacauccugggccuggacaucggcaucgccuccguggguggcgccaugguggagaucgacgaggaggagaaccccauccggc |
| | | ugaucgaccugggcgugcggguguucgagcgggccgaggugcccaagaccggcgacucccuggccauggcccgggcgcuggcccgguccgu |
| | | gcggcggcugacccggcggcgggcccaccggcugcugcgggcccggcggcugcugaagcgggagggcgugcugcaggccgccgacuucgac |
| | | gagaacggccugaucaagucccugcccaacacccccuggcagcugcgggccgccgcccuggaccggaagcugaccccccuggagugguccg |
| | | ccgugcugcugcaccugaucaagcaccggggcuaccugucccagcggaagaacgagggcgagaccgccgacaaggagcugggcgcccugcu |
| | | gaagggcguggccaacaacgcccacgcccugcagaccggcgacuuccggacccccgccgagcuggcccugaacaaguucgagaaggagucc |
| | | ggccacauccggaaccagcggggcgacuacucccacaccuucucccggaaggaccugcaggccgagcugauccugcuguucgagaagcaga |
| | | aggaguucggcaacccccacguguccggcggccugaaggagggcaucgagacccugcugaugacccagcggcccgcccuguccggcgacgc |
| | | cgugcagaagaugcugggccacugcaccuucgagcccgccgagcccaaggccgccaagaacaccuacaccgccgagcgguucaucuggcug |
| | | accaagcugaacaaccugcggauccuggagcagggcuccgagcggccccugaccgacaccgagcgggccacccugauggacgagcccuacc |
| | | ggaaguccaagcugaccuacgcccaggcccggaagcugcugggccuggaggacaccgccuucuucaagggccugcgguacggcaaggacaa |
| | | cgccgaggccuccacccugauggagaugaaggccuaccacgccaucucccgggcccuggagaaggagggccugaaggacaagaaguccccc |
| | | cugaaccuguccuccgagcugcaggacgagaucggcaccgccuucucccuguucaagaccgacgaggacaucaccggccggcugaaggacc |
| | | gggugcagcccgagauccuggaggcccugcugaagcacaucuccuucgacaaguucgugcagaucucccugaaggcccugcggcggaucgu |
| | | gccccugauggagcagggcaagcgguacgacgaggccugcgccgagaucuacggcgaccacuacggcaagaagaacaccgaggagaagauc |
| | | uaccugccccccauccccgccgacgagauccggaaccccguggugcugcgggcccugucccaggcccggaaggugaucaacggcguggugc |
| | | ggcgguacggcucccccgcccggauccacaucgagaccgcccgggaggugggcaaguccuucaaggaccggaaggagaucgagaagcggca |
| | | ggaggagaaccggaaggaccgggagaaggccgccgccaaguuccgggaguacuuccccaacuucgugggcgagcccaaguccaaggacauc |
| | | cugaagcugcggcuguacgagcagcagcacggcaagugccuguacuccggcaaggagaucaaccuggugcggcugaacgagaagggcuacg |
| | | uggagaucgaccacgcccugcccuucucccggaccugggacgacuccuucaacaacaaggugcuggugcugggcuccgagaaccagaacaa |
| | | gggcaaccagacccccuacgaguacuucaacggcaaggacaacucccgggaguggcaggaguucaaggcccggguggagaccucccgguuc |
| | | ccccgguccaagaagcagcggauccugcugcagaaguucgacgaggacggcuucaaggagugcaaccugaacgacacccgguacgugaacc |
| | | gcuuccugugccaguucguggccgaccacauccugcugaccggcaagggcaagcggggcguguucgccuccaacggccagaucaccaaccu |
| | | gcugcggggcuucuggggccugcggaaggugcgggccgagaacgaccggcaccacgcccuggacgccguggugguggccugcuccaccgug |
| | | gccaugcagcagaagaucacccgguucgugcgguacaaggagaugaacgccuucgacggcaagaccaucgacaaggagaccggcaaggugc |
| | | ugcaccagaagacccacuucccccagcccugggaguucuucgcccaggaggugaugauccggguguucggcaagcccgacggcaagcccga |
| | | guucgaggaggccgacacccccgagaagcugcggacccugcuggccgagaagcuguccucccggcccgaggccgugcacgaguacgugacc |
| | | ccccuguucgugucccgggcccccaaccggaagauguccggcgcccacaaggacacccugcgguccgccaagcgguucgugaagcacaacg |
| | | agaagaucuccgugaagcggguguggcugaccgagaucaagcuggccgaccuggagaacauggugaacuacaagaacggccgggagaucga |
| | | gcuguacgaggcccugaaggcccggcuggaggccuacggcggcaacgccaagcaggccuucgaccccaaggacaaccccuucuacaagaag |
| | | ggcggccagcuggugaaggccgugcggguggagaagacccaggaguccggcgugcugcugaacaagaagaacgccuacaccaucgccgaca |
| | | acggcgacauggugcggguggacguguucugcaagguggacaagaagggcaagaaccaguacuucaucgugcccaucuacgccuggcaggu |
| | | ggccgagaacauccugcccgacaucgacugcaagggcuaccggaucgacgacuccuacaccuucugcuucucccugcacaaguacgaccug |
| | | aucgccuuccagaaggacgagaaguccaagguggaguucgccuacuacaucaacugcgacuccuccaacggccgcguucuaccuggccugg |
| | | acgacaagggcuccaaggagcagcaguuccggaucuccacccagaaccuggugcugauccagaaguaccaggugaacgagcugggcaagga |
| | | gauccggcccugccggcugaagaagcggccccccgugcgguccggaaagcggaccgccgacggcuccgaguucgagucccccaagaagaag |
| | | cggaagguggaguag |
| 47 | R1333 | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | WT | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | Cas9- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | Cas9 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | fusion | GGASQEEFYKFIKPILEKMDGTEELLVKLNREDLLRKQRTFDNGSIPHQIHLGELHAILRRQEDFYPFLKDNREKIEKILTFRIPYYVGPL |
| | PA1-4 | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | (mRNA | AIVDLLFKTNRKVTVKQLKEDYFKKIECFDSVEISGVEDRFNASLGTYHDLLKIIKDKDFLDNEENEDILEDIVLTLTLFEDREMIEERLK |
| | N)- | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | amino | GSPAIKKGILQTVKVVDELVKVMGRHKPENIVIEMARENQTTQKGQKNSRERMKRIEEGIKELGSQILKEHPVENTQLQNEKLYLYYLQNG |
| | acid | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | sequence | LDKAGFIKRQLVETRQITKHVAQILDSRMNTKYDENDKLIREVKVITLKSKLVSDFRKDFQFYKVREINNYHHAHDAYLNAVVGTALIKKY |
| | (Example | PKLESEFVYGDYKVYDVRKMIAKSEQEIGKATAKYFFYSNIMNFFKTEITLANGEIRKRPLIETNGETGEIVWDKGRDFATVRKVLSMPQV |
| | 4) | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | PINYILGLDIGIASVGWAMVEIDEEENPIRLIDLGVRVFERAEVPKTGDSLAMARRLARSVRRLTRRRAHRLLRARRLLKREGVLQAADFD |
| | | ENGLIKSLPNTPWQLRAAALDRKLTPLEWSAVLLHLIKHRGYLSQRKNEGETADKELGALLKGVANNAHALQTGDFRTPAELALNKFEKES |
| | | GHIRNQRGDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSSELQDEIGTAFSLFKTDEDITGRLKDRVQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLVRLNEKGYVEIDHALPFSRTWDDSFNNKVLVLGSENQNKGNQTPYEYFNGKDNSREWQEFKARVETSRF |
| | | PRSKKQRILLQKFDEDGFKECNLNDTRYVNRFLCQFVADHILLTGKGKRRVFASNGQITNLLRGFWGLRKVRAENDRHHALDAVVVACSTV |
| | | AMQQKITRFVRYKEMNFDGKTIDKETGKVLHQKTHFPQPWEFFAQEVMIRVFGKPDGKPEFEEADTPEKLRTLLAEKLSSRPEAVHEYVT |
| | | PLFVSRAPNRKMSGAHKDTLRSAKRFVKHNEKISVKRVWLTEIKLADLENMVNYKNGREIELYEALKARLEAYGGNAKQAFDPKDNPFYKK |
| | | GGQLVKAVRVEKTQESGVLLNKKNAYTIADNGDMVRVDVFCKVDKKGKNQYFIVPIYAWQVAENILPDIDCKGYRIDDSYTFCFSLHKYDL |
| | | IAFQKDEKSKVEFAYYINCDSSNGRFYLAWHDKGSKEQQFRISTQNLVLIQKYQVNELGKEIRPCRLKKRPPVRSGKRTADGSEFESPKKK |
| | | RKVE* |
| 48 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 71 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | O)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGCGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCACCGAGGAGGGCACCUCC |
| | | ACCGAGUCCGAGGGCUCCGCCGGCACCUCCACCGAGUCCGAGGGCUCCGCCGGCACCUCCGAGUCCGCCACCGGCUCCUCCUCCGGUGCCG |
| | | CCUUCAAGCCCAACCCCAUCAACUACAUCCUGGGCCUGGACAUCGGCAUCGCCUCCGUGGGCUGGGCCAUGGUGGAGAUCGACGAGGAGGA |
| | | GAACCCCAUCCGGCUGAUCGACCUGGGCGUGCGGGUGUUCGAGCGGGCCGAGGUGCCCAAGACCGGCGACUCCCUGGCCAUGGCCCGGCGG |
| | | CUGGCCCGGUCCGUGCGGCGGCUGACCCGGCGGCGGGCCCACCGGCUGCUGCGGGCCCGGCGGCUGCUGAAGCGGGAGGGCGUGCUGCAGG |
| | | CCGCCGACUUCGACGAGAACGGCCUGAUCAAGUCCCUGCCCAACACCCCCUGGCAGCUGCGGGCCGCCGCCCUGGACCGGAAGCUGACCCC |
| | | CCUGGAGUGGUCCGCCGUGCUGCUGCACCUGAUCAAGCACCGGGGCUACCUGUCCCAGCGGAAGAACGAGGGCGAGACCGCCGACAAGGAG |
| | | CUGGGCGCCCUGCUGAAGGGCGUGGCCAACAACGCCCACGCCCUGCAGACCGGCGACUUCCGGACCCCCGCCGAGCUGGCCCUGAACAAGU |
| | | UCGAGAAGGAGUCCGGCCACAUCCGGAACCAGCGGGGCGACUACUCCCACACCUUCUCCCGGAAGGACCUGCAGGCCGAGCUGAUCCUGCU |
| | | GUUCGAGAAGCAGAAGGAGUUCGGCAACCCCCACGUGUCCGGCGGCCUGAAGGAGGGCAUCGAGACCCUGCUGAUGACCCAGCGGCCCGCC |
| | | CUGUCCGGCGACGCCGUGCAGAAGAUGCUGGGCCACUGCACCUUCGAGCCCGCCGAGCCCAAGGCCGCCAAGAACACCUACACCGCCGAGC |
| | | GGUUCAUCUGGCUGACCAAGCUGAACAACCUGCGGAUCCUGGAGCAGGGCUCCGAGCGGCCCCUGACCGACACCGAGCGGGCCACCCUGAU |
| | | GGACGAGCCCUACCGGAAGUCCAAGCUGACCUACGCCCAGGCCCGGAAGCUGCUGGGCCUGGAGGACACCGCCUUCUUCAAGGGCCUGCGG |
| | | UACGGCAAGGACAACGCCGAGGCCUCCACCCUGAUGGAGAUGAAGGCCUACCACGCCAUCUCCCGGGCCCUGGAGAAGGAGGGCCUGAAGG |
| | | ACAAGAAGUCCCCCCUGAACCUGUCCUCCGAGCUGCAGGACGAGAUCGGCACCGCCUUCUCCCUGUUCAAGACCGACGAGGACAUCACCGG |
| | | CCGGCUGAAGGACCGGGUGCAGCCCGAGAUCCUGGAGGCCCUGCUGAAGCACAUCUCCUUCGACAAGUUCGUGCAGAUCUCCCUGAAGGCC |
| | | CUGCGGCGGAUCGUGCCCCUGAUGGAGCAGGGCAAGCGGUACGACGAGGCCUGCGCCGAGAUCUACGGCGACCACUACGGCAAGAAGAACA |
| | | CCGAGGAGAAGAUCUACCUGCCCCCCAUCCCCGCCGACGAGAUCCGGAACCCCGUGGUGCUGCGGGCCCUGUCCCAGGCCCGGAAGGUGAU |
| | | CAACGGCGUGGUGCGGCGGUACGGCUCCCCCGCCCGGAUCCACAUCGAGACCGCCCGGGAGGUGGGCAAGUCCUUCAAGGACCGGAAGGAG |
| | | AUCGAGAAGCGGCAGGAGGAGAACCGGAAGGACCGGGAGAAGGCCGCCGCCAAGUUCCGGGAGUACUUCCCCAACUUCGUGGGCGAGCCCA |
| | | AGUCCAAGGACAUCCUGAAGCUGCGGCUGUACGAGCAGCAGCACGGCAAGUGCCUGUACUCCGGCAAGGAGAUCAACCUGGUGCGGCUGAA |
| | | CGAGAAGGGCUACGUGGAGAUCGACCACGCCCUGCCCUUCUCCCGGACCUGGGACGACUCCUUCAACAACAAGGUGCUGGUGCUGGGCUCC |
| | | GAGAACCAGAACAAGGGCAACCAGACCCCCUACGAGUACUUCAACGGCAAGGACAACUCCCGGGAGUGGCAGGAGUUCAAGGCCCGGGUGG |
| | | AGACCUCCCGGUUCCCCCGGUCCAAGAAGCAGCGGAUCCUGCUGCAGAAGUUCGACGAGGACGGCUUCAAGGAGUGCAACCUGAACGACAC |
| | | CCGGUACGUGAACCGCUUCCUGUGCCAGUUCGUGGCCGACCACAUCCUGCUGACCGGCAAGGGCAAGCGGCGGGUGUUCGCCUCCAACGGC |
| | | CAGAUCACCAACCUGCUGCGGGGCUUCUGGGGCCUGCGGAAGGUGCGGGCCGAGAACGACCGGCACCACGCCCUGGACGCCGUGGUGGUGG |
| | | CCUGCUCCACCGUGGCCAUGCAGCAGAAGAUCACCCGGUUCGUGCGGUACAAGGAGAUGAACGCCUUCGACGGCAAGACCAUCGACAAGGA |
| | | GACCGGCAAGGUGCUGCACCAGAAGACCCACUUCCCCCAGCCCUGGGAGUUCUUCGCCCAGGAGGUGAUGAUCCGGGUGUUCGGCAAGCCC |
| | | GACGGCAAGCCCGAGUUCGAGGAGGCCGACACCCCCGAGAAGCUGCGGACCCUGCUGGCCGAGAAGCUGUCCUCCCGGCCCGAGGCCGUGC |
| | | ACGAGUACGUGACCCCCCUGUUCGUGUCCCGGGCCCCCAACCGGAAGAUGUCCGGCGCCCACAAGGACACCCUGCGGUCCGCCAAGCGGUU |
| | | CGUGAAGCACAACGAGAAGAUCUCCGUGAAGCGGGUGUGGCUGACCGAGAUCAAGCUGGCCGACCUGGAGAACAUGGUGAACUACAAGAAC |
| | | GGCCGGGAGAUCGAGCUGUACGAGGCCCUGAAGGCCCGGCUGGAGGCCUACGGCGGCAACGCCAAGCAGGCCUUCGACCCCAAGGACAACC |
| | | CCUUCUACAAGAAGGGCGGCCAGCUGGUGAAGGCCGUGCGGGUGGAGAAGACCCAGGAGUCCGGCGUGCUGCUGAACAAGAAGAACGCCUA |
| | | CACCAUCGCCGACAACGGCGACAUGGUGCGGGUGGACGUGUUCUGCAAGGUGGACAAGAAGGGCAAGAACCAGUACUUCAUCGUGCCCAUC |
| | | UACGCCUGGCAGGUGGCCGAGAACAUCCUGCCCGACAUCGACUGCAAGGGCUACCGGAUCGACGACUCCUACACCUUCUGCUUCUCCCUGC |
| | | ACAAGUACGACCUGAUCGCCUUCCAGAAGGACGAGAAGUCCAAGGUGGAGUUCGCCUACUACAUCAACUGCGACUCCUCCAACGGCCGGUU |
| | | CUACCUGGCCUGGCACGACAAGGGCUCCAAGGAGCAGCAGUUCCGGAUCUCCACCCAGAACCUGGUGCUGAUCCAGAAGUACCAGGUGAAC |
| | | GAGCUGGGCAAGGAGAUCCGGCCCUGCCGGCUGAAGAAGCGGCCCCCCGUGCGGUCCGGAAAGCGGACCGCCGACGGCUCCGGAGGAGGAA |
| | | GCCCGAAGAAGAAGAGAAAGGUCAGCGAAAGCGCAACACCGGAAAGCGUCAGCGGAUGGAGACUGUUCAAGAAGAUCAGCUAGCUAGCACC |
| | | AGCCUCAAGAACACCCGAAUGGAGUCUCUAAGCUACAUAAUACCAACUUACACUUUACAAAAUGUUGUCCCCCAAAAUGUAGCCAUUCGUA |
| | | UCUGCUCCUAAUAAAAAGAAAGUUUCUUCACAUUCUCUCGAGAAAAAAAAAAAAUGGAAAAAAAAAAAACGGAAAAAAAAAAAAGGUAAAA |
| | | AAAAAAAAUAUAAAAAAAAAAAACAUAAAAAAAAAAAACGAAAAAAAAAAAACGUAAAAAAAAAAAACUCAAAAAAAAAAAAGAUAAAAAA |
| | | AAAAAACCUAAAAAAAAAAAAUGUAAAAAAAAAAAAGGGAAAAAAAAAAAACGCAAAAAAAAAAAACACAAAAAAAAAAAAUGCAAAAAAA |
| | | AAAAAUCGAAAAAAAAAAAAUCUAAAAAAAAAAAACGAAAAAAAAAAAACCCAAAAAAAAAAAAGACAAAAAAAAAAAAUAGAAAAAAAAA |
| | | AAAGUUAAAAAAAAAAAACUGAAAAAAAAAAAAUUUAAAAAAAAAAAAUCUAG |
| 49 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 71 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | O)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCGCCGGCACCUCCACCGAGUCCGAGGG |
| | | CUCCGCCGGCACCUCCGAGUCCGCCACCGGCUCCUCCUCCGGUGCCGCCUUCAAGCCCAACCCCAUCAACUACAUCCUGGGCCUGGACAUC |
| | | GGCAUCGCCUCCGUGGGCUGGGCCAUGGUGGAGAUCGACGAGGAGGAGAACCCCAUCCGGCUGAUCGACCUGGGCGUGCGGGUGUUCGAGC |
| | | GGGCCGAGGUGCCCAAGACCGGCGACUCCCUGGCCAUGGCCCGGCGGCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCAGGCCGCCGACUUCGACGAGAACGGCCUGAUCAAGUCCCUGCCCAAC |
| | | ACCCCCUGGCAGCUGCGGGCCGCCGCCCUGGACCGGAAGCUGACCCCCCUGGAGUGGUCCGCCGUGCUGCUGCACCUGAUCAAGCACCGGG |
| | | GCUACCUGUCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGGGCGACUAC |
| | | UCCCACACCUUCUCCCGGAAGGACCUGCAGGCCGAGCUGAUCCUGCUGUUCGAGAAGCAGAAGGAGUUCGGCAACCCCCACGUGUCCGGCG |
| | | GCCUGAAGGAGGGCAUCGAGACCCUGCUGAUGACCCAGCGGCCCGCCCUGUCCGGCGACGCCGUGCAGAAGAUGCUGGGCCACUGCACCUU |
| | | CGAGCCCGCCGAGCCCAAGGCCGCCAAGAACACCUACACCGCCGAGCGGUUCAUCUGGCUGACCAAGCUGAACAACCUGCGGAUCCUGGAG |
| | | CAGGGCUCCGAGCGGCCCCUGACCGACACCGAGCGGGCCACCCUGAUGGACGAGCCCUACCGGAAGUCCAAGCUGACCUACGCCCAGGCCC |
| | | GGAAGCUGCUGGGCCUGGAGGACACCGCCUUCUUCAAGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAGUCCCCCCUGAACCUGUCCUCCGAGCUGCAGGACGAG |
| | | AUCGGCACCGCCUUCUCCCUGUUCAAGACCGACGAGGACAUCACCGGCCGGCUGAAGGACCGGGUGCAGCCCGAGAUCCUGGAGGCCCUGC |
| | | UGAAGCACAUCUCCUUCGACAAGUUCGUGCAGAUCUCCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAGAAGAUCUACCUGCCCCCCAUCCCCGCCGACGAGAUC |
| | | CGGAACCCCGUGGUGCUGCGGGCCCUGUCCCAGGCCCGGAAGGUGAUCAACGGCGUGGUGCGGCGGUACGGCUCCCCCGCCCGGAUCCACA |
| | | UCGAGACCGCCCGGGAGGUGGGCAAGUCCUUCAAGGACCGGAAGGAGAUCGAGAAGCGGCAGGAGGAGAACCGGAAGGACCGGGAGAAGGC |
| | | CGCCGCCAAGUUCCGGGAGUACUUCCCCAACUUCGUGGGCGAGCCCAAGUCCAAGGACAUCCUGAAGCUGCGGCUGUACGAGCAGCAGCAC |
| | | GGCAAGUGCCUGUACUCCGGCAAGGAGAUCAACCUGGUGCGGCUGAACGAGAAGGGCUACGUGGAGAUCGACCACGCCCUGCCCUUCUCCC |
| | | GGACCUGGGACGACUCCUUCAACAACAAGGUGCUGGUGCUGGGCUCCGAGAACCAGAACAAGGGCAACCAGACCCCCUACGAGUACUUCAA |
| | | CGGCAAGGACAACUCCCGGGAGUGGCAGGAGUUCAAGGCCCGGGUGGAGACCUCCCGGUUCCCCCGGUCCAAGAAGCAGCGGAUCCUGCUG |
| | | CAGAAGUUCGACGAGGACGGCUUCAAGGAGUGCAACCUGAACGACACCCGGUACGUGAACCGCUUCCUGUGCCAGUUCGUGGCCGACCACA |
| | | UCCUGCUGACCGGCAAGGGCAAGCGGCGGGUGUUCGCCUCCAACGGCCAGAUCACCAACCUGCUGCGGGGCUUCUGGGGCCUGCGGAAGGU |
| | | GCGGGCCGAGAACGACCGGCACCACGCCCUGGACGCCGUGGUGGUGGCCUGCUCCACCGUGGCCAUGCAGCAGAAGAUCACCCGGUUCGUG |
| | | CGGUACAAGGAGAUGAACGCCUUCGACGGCAAGACCAUCGACAAGGAGACCGGCAAGGUGCUGCACCAGAAGACCCACUUCCCCCAGCCCU |
| | | GGGAGUUCUUCGCCCAGGAGGUGAUGAUCCGGGUGUUCGGCAAGCCCGACGGCAAGCCCGAGUUCGAGGAGGCCGACACCCCCGAGAAGCU |
| | | GCGGACCCUGCUGGCCGAGAAGCUGUCCUCCCGGCCCGAGGCCGUGCACGAGUACGUGACCCCCCUGUUCGUGUCCCGGGCCCCCAACCGG |
| | | AAGAUGUCCGGCGCCCACAAGGACACCCUGCGGUCCGCCAAGCGGUUCGUGAAGCACAACGAGAAGAUCUCCGUGAAGCGGGUGUGGCUGA |
| | | CCGAGAUCAAGCUGGCCGACCUGGAGAACAUGGUGAACUACAAGAACGGCCGGGAGAUCGAGCUGUACGAGGCCCUGAAGGCCCGGCUGGA |
| | | GGCCUACGGCGGCAACGCCAAGCAGGCCUUCGACCCCAAGGACAACCCCUUCUACAAGAAGGGCGGCCAGCUGGUGAAGGCCGUGCGGGUG |
| | | GAGAAGACCCAGGAGUCCGGCGUGCUGCUGAACAAGAAGAACGCCUACACCAUCGCCGACAACGGCGACAUGGUGCGGGUGGACGUGUUCU |
| | | GCAAGGUGGACAAGAAGGGCAAGAACCAGUACUUCAUCGUGCCCAUCUACGCCUGGCAGGUGGCCGAGAACAUCCUGCCCGACAUCGACUG |
| | | CAAGGGCUACCGGAUCGACGACUCCUACACCUUCUGCUUCUCCCUGCACAAGUACGACCUGAUCGCCUUCCAGAAGGACGAGAAGUCCAAG |
| | | GUGGAGUUCGCCUACUACAUCAACUGCGACUCCUCCAACGGCCGGUUCUACCUGGCCUGGCACGACAAGGGCUCCAAGGAGCAGCAGUUCC |
| | | GGAUCUCCACCCAGAACCUGGUGCUGAUCCAGAAGUACCAGGUGAACGAGCUGGGCAAGGAGAUCCGGCCCUGCCGGCUGAAGAAGCGGCC |
| | | CCCCGUGCGGUCCGGAAAGCGGACCGCCGACGGCUCCGGAGGAGGAAGCCCGAAGAAGAAGAGAAAGGUCAGCGAAAGCGCAACACCGGAA |
| | | AGCGUCAGCGGAUGGAGACUGUUCAAGAAGAUCAGCUAG |
| 50 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 71 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | O)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATESGGTSTESEGSAGTSTESEGSAGSAGSTSTEEGTSTESEGSAGTSTESEGSAGTSESATGSSSGAAFKPNPINYILGLDI |
| | | GIASVGWAMVEIDEEENPIRLIDLGVRVFERAEVPKTGDSLAMARRLARSVRRLTRRRAHRLLRARRLLKREGVLQAADFDENGLIKSLPN |
| | | TPWQLRAAALDRKLTPLEWSAVLLHLIKHRGYLSQRKNEGETADKELGALLKGVANNAHALQTGDFRTPAELALNKFEKESGHIRNQRGDY |
| | | SHTFSRKDLQAELILLFEKQKEFGNPHVSGGLKEGIETLLMTQRPALSGDAVQKMLGHCTFEPAEPKAAKNTYTAERFIWLTKLNNLRILE |
| | | QGSERPLTDTERATLMDEPYRKSKLTYAQARKLLGLEDTAFFKGLRYGKDNAEASTLMEMKAYHAISRALEKEGLKDKKSPLNLSSELQDE |
| | | IGTAFSLFKTDEDITGRLKDRVQPEILEALLKHISFDKFVQISLKALRRIVPLMEQGKRYDEACAEIYGDHYGKKNTEEKIYLPPIPADEI |
| | | RNPVVLRALSQARKVINGVVRRYGSPARIHIETAREVGKSFKDRKEIEKRQEENRKDREKAAAKFREYFPNFVGEPKSKDILKLRLYEQQH |
| | | GKCLYSGKEINLVRLNEKGYVEIDHALPFSRTWDDSFNNKVLVLGSENQNKGNQTPYEYFNGKDNSREWQEFKARVETSRFPRSKKQRILL |
| | | QKFDEDGFKECNLNDTRYVNRFLCQFVADHILLTGKGKRRVFASNGQITNLLRGFWGLRKVRAENDRHHALDAVVVACSTVAMQQKITRFV |
| | | RYKEMNAFDGKTIDKETGKVLHQKTHFPQPWEFFAQEVMIRVFGKPDGKPEFEEADTPEKLRTLLAEKLSSRPEAVHEYVTPLFVSRAPNR |
| | | KMSGAHKDTLRSAKRFVKHNEKISVKRVWLTEIKLADLENMVNYKNGREIELYEALKARLEAYGGNAKQAFDPKDNPFYKKGGQLVKAVRV |
| | | EKTQESGVLLNKKNAYTIADNGDMVRVDVFCKVDKKGKNQYFIVPIYAWQVAENILPDIDCKGYRIDDSYTFCFSLHKYDLIAFQKDEKSK |
| | | VEFAYYINCDSSNGRFYLAWHDKGSKEQQFRISTQNLVLIQKYQVNELGKEIRPCRLKKRPPVRSGKRTADGSGGGSPKKKRKVSESATPE |
| | | SVSGWRLFKKIS* |
| 51 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 71 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | O)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCGCCGGCACCUCCACCGAGUCCGAGGG |
| | | CUCCGCCGGCACCUCCGAGUCCGCCACCGGCUCCUCCUCCGGUGCCGCCUUCAAGCCCAACCCCAUCAACUACAUCCUGGGCCUGGACAUC |
| | | GGCAUCGCCUCCGUGGGCUGGGCCAUGGUGGAGAUCGACGAGGAGGAGAACCCCAUCCGGCUGAUCGACCUGGGCGUGCGGGUGUUCGAGC |
| | | GGGCCGAGGUGCCCAAGACCGGCGACUCCCUGGCCAUGGCCCGGCGGCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCAGGCCGCCGACUUCGACGAGAACGGCCUGAUCAAGUCCCUGCCCAAC |
| | | ACCCCCUGGCAGCUGCGGGCCGCCGCCCUGGACCGGAAGCUGACCCCCCUGGAGUGGUCCGCCGUGCUGCUGCACCUGAUCAAGCACCGGG |
| | | GCUACCUGUCCCAGCGGAAGAACGAGGGCGAGACCGCCGACAAGGAGCUGGGCGCCCUGCUGAAGGGCGUGGCCAACAACGCCCACGCCCU |
| | | GCAGACCGGCGACUUCCGGACCCCCGCCGAGCUGGCCCUGAACAAGUUCGAGAAGGAGUCCGGCCACAUCCGGAACCAGCGGGGCGACUAC |
| | | UCCCACACCUUCUCCCGGAAGGACCUGCAGGCCGAGCUGAUCCUGCUGUUCGAGAAGCAGAAGGAGUUCGGCAACCCCCACGUGUCCGGCG |
| | | GCCUGAAGGAGGGCAUCGAGACCCUGCUGAUGACCCAGCGGCCCGCCCUGUCCGGCGACGCCGUGCAGAAGAUGCUGGGCCACUGCACCUU |
| | | CGAGCCCGCCGAGCCCAAGGCCGCCAAGAACACCUACACCGCCGAGCGGUUCAUCUGGCUGACCAAGCUGAACAACCUGCGGAUCCUGGAG |
| | | CAGGGCUCCGAGCGGCCCCUGACCGACACCGAGCGGGCCACCCUGAUGGACGAGCCCUACCGGAAGUCCAAGCUGACCUACGCCCAGGCCC |
| | | GGAAGCUGCUGGGCCUGGAGGACACCGCCUUCUUCAAGGGCCUGCGGUACGGCAAGGACAACGCCGAGGCCUCCACCCUGAUGGAGAUGAA |
| | | GGCCUACCACGCCAUCUCCCGGGCCCUGGAGAAGGAGGGCCUGAAGGACAAGAAGUCCCCCCUGAACCUGUCCUCCGAGCUGCAGGACGAG |
| | | AUCGGCACCGCCUUCUCCCUGUUCAAGACCGACGAGGACAUCACCGGCCGGCUGAAGGACCGGGUGCAGCCCGAGAUCCUGGAGGCCCUGC |
| | | UGAAGCACAUCUCCUUCGACAAGUUCGUGCAGAUCUCCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAGAAGAUCUACCUGCCCCCCAUCCCCGCCGACGAGAUC |
| | | CGGAACCCCGUGGUGCUGCGGGCCCUGUCCCAGGCCCGGAAGGUGAUCAACGGCGUGGUGCGGCGGUACGGCUCCCCCGCCCGGAUCCACA |
| | | UCGAGACCGCCCGGGAGGUGGGCAAGUCCUUCAAGGACCGGAAGGAGAUCGAGAAGCGGCAGGAGGAGAACCGGAAGGACCGGGAGAAGGC |
| | | CGCCGCCAAGUUCCGGGAGUACUUCCCCAACUUCGUGGGCGAGCCCAAGUCCAAGGACAUCCUGAAGCUGCGGCUGUACGAGCAGCAGCAC |
| | | GGCAAGUGCCUGUACUCCGGCAAGGAGAUCAACCUGGUGCGGCUGAACGAGAAGGGCUACGUGGAGAUCGACCACGCCCUGCCCUUCUCCC |
| | | GGACCUGGGACGACUCCUUCAACAACAAGGUGCUGGUGCUGGGCUCCGAGAACCAGAACAAGGGCAACCAGACCCCCUACGAGUACUUCAA |
| | | CGGCAAGGACAACUCCCGGGAGUGGCAGGAGUUCAAGGCCCGGGUGGAGACCUCCCGGUUCCCCCGGUCCAAGAAGCAGCGGAUCCUGCUG |
| | | CAGAAGUUCGACGAGGACGGCUUCAAGGAGUGCAACCUGAACGACACCCGGUACGUGAACCGCUUCCUGUGCCAGUUCGUGGCCGACCACA |
| | | UCCUGCUGACCGGCAAGGGCAAGCGGCGGGUGUUCGCCUCCAACGGCCAGAUCACCAACCUGCUGCGGGGCUUCUGGGGCCUGCGGAAGGU |
| | | GCGGGCCGAGAACGACCGGCACCACGCCCUGGACGCCGUGGUGGUGGCCUGCUCCACCGUGGCCAUGCAGCAGAAGAUCACCCGGUUCGUG |
| | | CGGUACAAGGAGAUGAACGCCUUCGACGGCAAGACCAUCGACAAGGAGACCGGCAAGGUGCUGCACCAGAAGACCCACUUCCCCCAGCCCU |
| | | GGGAGUUCUUCGCCCAGGAGGUGAUGAUCCGGGUGUUCGGCAAGCCCGACGGCAAGCCCGAGUUCGAGGAGGCCGACACCCCCGAGAAGCU |
| | | GCGGACCCUGCUGGCCGAGAAGCUGUCCUCCCGGCCCGAGGCCGUGCACGAGUACGUGACCCCCCUGUUCGUGUCCCGGGCCCCCAACCGG |
| | | AAGAUGUCCGGCGCCCACAAGGACACCCUGCGGUCCGCCAAGCGGUUCGUGAAGCACAACGAGAAGAUCUCCGUGAAGCGGGUGUGGCUGA |
| | | CCGAGAUCAAGCUGGCCGACCUGGAGAACAUGGUGAACUACAAGAACGGCCGGGAGAUCGAGCUGUACGAGGCCCUGAAGGCCCGGCUGGA |
| | | GGCCUACGGCGGCAACGCCAAGCAGGCCUUCGACCCCAAGGACAACCCCUUCUACAAGAAGGGCGGCCAGCUGGUGAAGGCCGUGCGGGUG |
| | | GAGAAGACCCAGGAGUCCGGCGUGCUGCUGAACAAGAAGAACGCCUACACCAUCGCCGACAACGGCGACAUGGUGCGGGUGGACGUGUUCU |
| | | GCAAGGUGGACAAGAAGGGCAAGAACCAGUACUUCAUCGUGCCCAUCUACGCCUGGCAGGUGGCCGAGAACAUCCUGCCCGACAUCGACUG |
| | | CAAGGGCUACCGGAUCGACGACUCCUACACCUUCUGCUUCUCCCUGCACAAGUACGACCUGAUCGCCUUCCAGAAGGACGAGAAGUCCAAG |
| | | GUGGAGUUCGCCUACUACAUCAACUGCGACUCCUCCAACGGCCGGUUCUACCUGGCCUGGCACGACAAGGGCUCCAAGGAGCAGCAGUUCC |
| | | GGAUCUCCACCCAGAACCUGGUGCUGAUCCAGAAGUACCAGGUGAACGAGCUGGGCAAGGAGAUCCGGCCCUGCCGGCUGAAGAAGCGGCC |
| | | CCCCGUGCGGUCCGGAAAGCGGACCGCCGACGGCUCCGGAGGAGGAAGCCCGAAGAAGAAGAGAAAGGUCUAG |
| 52 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9- | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 71 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | O)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | mRNA | LDKAGFIKRQLVETRQITKHVAQILDSRMNTKYDENDKLIREVKVITLKSKLVSDFRKDFQFYKVREINNYHHAHDAYLNAVVGTALIKKY |
| | sequence | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATGSSSGAAFKPNPINYILGLDI |
| | | GIASVGWAMVEIDEEENPIRLIDLGVRVFERAEVPKTGDSLAMARRLARSVRRLTRRRAHRLLRARRLLKREGVLQAADFDENGLIKSLPN |
| | | TPWQLRAAALDRKLTPLEWSAVLLHLIKHRGYLSQRKNEGETADKELGALLKGVANNAHALQTGDFRTPAELALNKFEKESGHIRNQRGDY |
| | | SHTFSRKDLQAELILLFEKQKEFGNPHVSGGLKEGIETLLMTQRPALSGDAVQKMLGHCTFEPAEPKAAKNTYTAERFIWLTKLNNLRILE |
| | | QGSERPLTDTERATLMDEPYRKSKLTYAQARKLLGLEDTAFFKGLRYGKDNAEASTLMEMKAYHAISRALEKEGLKDKKSPLNLSSELQDE |
| | | IGTAFSLFKTDEDITGRLKDRVQPEILEALLKHISFDKFVQISLKALRRIVPLMEQGKRYDEACAEIYGDHYGKKNTEEKIYLPPIPADEI |
| | | RNPVVLRALSQARKVINGVVRRYGSPARIHIETAREVGKSFKDRKEIEKRQEENRKDREKAAAKFREYFPNFVGEPKSKDILKLRLYEQQH |
| | | GKCLYSGKEINLVRLNEKGYVEIDHALPFSRTWDDSFNNKVLVLGSENQNKGNQTPYEYFNGKDNSREWQEFKARVETSRFPRSKKQRILL |
| | | QKFDEDGFKECNLNDTRYVNRFLCQFVADHILLTGKGKRRVFASNGQITNLLRGFWGLRKVRAENDRHHALDAVVVACSTVAMQQKITRFV |
| | | RYKEMNAFDGKTIDKETGKVLHQKTHFPQPWEFFAQEVMIRVFGKPDGKPEFEEADTPEKLRTLLAEKLSSRPEAVHEYVTPLFVSRAPNR |
| | | KMSGAHKDTLRSAKRFVKHNEKISVKRVWLTEIKLADLENMVNYKNGREIELYEALKARLEAYGGNAKQAFDPKDNPFYKKGGQLVKAVRV |
| | | EKTQESGVLLNKKNAYTIADNGDMVRVDVFCKVDKKGKNQYFIVPIYAWQVAENILPDIDCKGYRIDDSYTFCFSLHKYDLIAFQKDEKSK |
| | | VEFAYYINCDSSNGRFYLAWHDKGSKEQQFRISTQNLVLIQKYQVNELGKEIRPCRLKKRPPVRSGKRTADGSGGGSPKKKRKV* |
| 53 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 61 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | P)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCACCGAGGAGGGCACCUCC |
| | | ACCGAGUCCGAGGGCUCCGCCGGCACCUCCACCGAGUCCGAGGGCUCCUCCUCCGGUGCCGCCUUCAAGCCCAACCCCAUCAACUACAUCC |
| | | UGGGCCUGGACAUCGGCAUCGCCUCCGUGGGCUGGGCCAUGGUGGAGAUCGACGAGGAGGAGAACCCCAUCCGGCUGAUCGACCUGGGCGU |
| | | GCGGGUGUUCGAGCGGGCCGAGGUGCCCAAGACCGGCGACUCCCUGGCCAUGGCCCGGCGGCUGGCCCGGUCCGUGCGGCGGCUGACCCGG |
| | | CGGCGGGCCCACCGGCUGCUGCGGGCCCGGCGGCUGCUGAAGCGGGAGGGCGUGCUGCAGGCCGCCGACUUCGACGAGAACGGCCUGAUCA |
| | | AGUCCCUGCCCAACACCCCCUGGCAGCUGCGGGCCGCCGCCCUGGACCGGAAGCUGACCCCCCUGGAGUGGUCCGCCGUGCUGCUGCACCU |
| | | GAUCAAGCACCGGGGCUACCUGUCCCAGCGGAAGAACGAGGGCGAGACCGCCGACAAGGAGCUGGGCGCCCUGCUGAAGGGCGUGGCCAAC |
| | | AACGCCCACGCCCUGCAGACCGGCGACUUCCGGACCCCCGCCGAGCUGGCCCUGAACAAGUUCGAGAAGGAGUCCGGCCACAUCCGGAACC |
| | | AGCGGGGCGACUACUCCCACACCUUCUCCCGGAAGGACCUGCAGGCCGAGCUGAUCCUGCUGUUCGAGAAGCAGAAGGAGUUCGGCAACCC |
| | | CCACGUGUCCGGCGGCCUGAAGGAGGGCAUCGAGACCCUGCUGAUGACCCAGCGGCCCGCCCUGUCCGGCGACGCCGUGCAGAAGAUGCUG |
| | | GGCCACUGCACCUUCGAGCCCGCCGAGCCCAAGGCCGCCAAGAACACCUACACCGCCGAGCGGUUCAUCUGGCUGACCAAGCUGAACAACC |
| | | UGCGGAUCCUGGAGCAGGGCUCCGAGCGGCCCCUGACCGACACCGAGCGGGCCACCCUGAUGGACGAGCCCUACCGGAAGUCCAAGCUGAC |
| | | CUACGCCCAGGCCCGGAAGCUGCUGGGCCUGGAGGACACCGCCUUCUUCAAGGGCCUGCGGUACGGCAAGGACAACGCCGAGGCCUCCACC |
| | | CUGAUGGAGAUGAAGGCCUACCACGCCAUCUCCCGGGCCCUGGAGAAGGAGGGCCUGAAGGACAAGAAGUCCCCCCUGAACCUGUCCUCCG |
| | | AGCUGCAGGACGAGAUCGGCACCGCCUUCUCCCUGUUCAAGACCGACGAGGACAUCACCGGCCGGCUGAAGGACCGGGUGCAGCCCGAGAU |
| | | CCUGGAGGCCCUGCUGAAGCACAUCUCCUUCGACAAGUUCGUGCAGAUCUCCCUGAAGGCCCUGCGGCGGAUCGUGCCCCUGAUGGAGCAG |
| | | GGCAAGCGGUACGACGAGGCCUGCGCCGAGAUCUACGGCGACCACUACGGCAAGAAGAACACCGAGGAGAAGAUCUACCUGCCCCCCAUCC |
| | | CCGCCGACGAGAUCCGGAACCCCGUGGUGCUGCGGGCCCUGUCCCAGGCCCGGAAGGUGAUCAACGGCGUGGUGCGGCGGUACGGCUCCCC |
| | | CGCCCGGAUCCACAUCGAGACCGCCCGGGAGGUGGGCAAGUCCUUCAAGGACCGGAAGGAGAUCGAGAAGCGGCAGGAGGAGAACCGGAAG |
| | | GACCGGGAGAAGGCCGCCGCCAAGUUCCGGGAGUACUUCCCCAACUUCGUGGGCGAGCCCAAGUCCAAGGACAUCCUGAAGCUGCGGCUGU |
| | | ACGAGCAGCAGCACGGCAAGUGCCUGUACUCCGGCAAGGAGAUCAACCUGGUGCGGCUGAACGAGAAGGGCUACGUGGAGAUCGACCACGC |
| | | CCUGCCCUUCUCCCGGACCUGGGACGACUCCUUCAACAACAAGGUGCUGGUGCUGGGCUCCGAGAACCAGAACAAGGGCAACCAGACCCCC |
| | | UACGAGUACUUCAACGGCAAGGACAACUCCCGGGAGUGGCAGGAGUUCAAGGCCCGGGUGGAGACCUCCCGGUUCCCCCGGUCCAAGAAGC |
| | | AGCGGAUCCUGCUGCAGAAGUUCGACGAGGACGGCUUCAAGGAGUGCAACCUGAACGACACCCGGUACGUGAACCGCUUCCUGUGCCAGUU |
| | | CGUGGCCGACCACAUCCUGCUGACCGGCAAGGGCAAGCGGCGGGUGUUCGCCUCCAACGGCCAGAUCACCAACCUGCUGCGGGGCUUCUGG |
| | | GGCCUGCGGAAGGUGCGGGCCGAGAACGACCGGCACCACGCCCUGGACGCCGUGGUGGUGGCCUGCUCCACCGUGGCCAUGCAGCAGAAGA |
| | | UCACCCGGUUCGUGCGGUACAAGGAGAUGAACGCCUUCGACGGCAAGACCAUCGACAAGGAGACCGGCAAGGUGCUGCACCAGAAGACCCA |
| | | CUUCCCCCAGCCCUGGGAGUUCUUCGCCCAGGAGGUGAUGAUCCGGGUGUUCGGCAAGCCCGACGGCAAGCCCGAGUUCGAGGAGGCCGAC |
| | | ACCCCCGAGAAGCUGCGGACCCUGCUGGCCGAGAAGCUGUCCUCCCGGCCCGAGGCCGUGCACGAGUACGUGACCCCCCUGUUCGUGUCCC |
| | | GGGCCCCCAACCGGAAGAUGUCCGGCGCCCACAAGGACACCCUGCGGUCCGCCAAGCGGUUCGUGAAGCACAACGAGAAGAUCUCCGUGAA |
| | | GCGGGUGUGGCUGACCGAGAUCAAGCUGGCCGACCUGGAGAACAUGGUGAACUACAAGAACGGCCGGGAGAUCGAGCUGUACGAGGCCCUG |
| | | AAGGCCCGGCUGGAGGCCUACGGCGGCAACGCCAAGCAGGCCUUCGACCCCAAGGACAACCCCUUCUACAAGAAGGGCGGCCAGCUGGUGA |
| | | AGGCCGUGCGGGUGGAGAAGACCCAGGAGUCCGGCGUGCUGCUGAACAAGAAGAACGCCUACACCAUCGCCGACAACGGCGACAUGGUGCG |
| | | GGUGGACGUGUUCUGCAAGGUGGACAAGAAGGGCAAGAACCAGUACUUCAUCGUGCCCAUCUACGCCUGGCAGGUGGCCGAGAACAUCCUG |
| | | CCCGACAUCGACUGCAAGGGCUACCGGAUCGACGACUCCUACACCUUCUGCUUCUCCCUGCACAAGUACGACCUGAUCGCCUUCCAGAAGG |
| | | ACGAGAAGUCCAAGGUGGAGUUCGCCUACUACAUCAACUGCGACUCCUCCAACGGCCGGUUCUACCUGGCCUGGCACGACAAGGGCUCCAA |
| | | GGAGCAGCAGUUCCGGAUCUCCACCCAGAACCUGGUGCUGAUCCAGAAGUACCAGGUGAACGAGCUGGGCAAGGAGAUCCGGCCCUGCCGG |
| | | CUGAAGAAGCGGCCCCCCGUGCGGUCCGGAAAGCGGACCGCCGACGGCUCCGGAGGAGGAAGCCCGAAGAAGAAGAGAAAGGUCAGCGAAA |
| | | GCGCAACACCGGAAAGCGUCAGCGGAUGGAGACUGUUCAAGAAGAUCAGCUAGCUAGCACCAGCCUCAAGAACACCCGAAUGGAGUCUCUA |
| | | AGCUACAUAAUACCAACUUACACUUUACAAAAUGUUGUCCCCCAAAAUGUAGCCAUUCGUAUCUGCUCCUAAUAAAAAGAAAGUUUCUUCA |
| | | CAUUCUCUCGAGAAAAAAAAAAAAUGGAAAAAAAAAAAACGGAAAAAAAAAAAAGGUAAAAAAAAAAAAUAUAAAAAAAAAAAACAUAAAA |
| | | AAAAAAAACGAAAAAAAAAAAACGUAAAAAAAAAAAACUCAAAAAAAAAAAAGAUAAAAAAAAAAAACCUAAAAAAAAAAAAUGUAAAAAA |
| | | AAAAAAGGGAAAAAAAAAAAACGCAAAAAAAAAAAACACAAAAAAAAAAAAUGCAAAAAAAAAAAAUCGAAAAAAAAAAAAUCUAAAAAAA |
| | | AAAAACGAAAAAAAAAAAACCCAAAAAAAAAAAAGACAAAAAAAAAAAAUAGAAAAAAAAAAAAGUUAAAAAAAAAAAACUGAAAAAAAAA |
| | | AAAUUUAAAAAAAAAAAAUCUAG |
| 54 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 61 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | P)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCGCCGGCACCUCCACCGAGUCCGAGGG |
| | | CUCCUCCUCCGGUGCCGCCUUCAAGCCCAACCCCAUCAACUACAUCCUGGGCCUGGACAUCGGCAUCGCCUCCGUGGGCUGGGCCAUGGUG |
| | | GAGAUCGACGAGGAGGAGAACCCCAUCCGGCUGAUCGACCUGGGCGUGCGGGUGUUCGAGCGGGCCGAGGUGCCCAAGACCGGCGACUCCC |
| | | UGGCCAUGGCCCGGCGGCUGGCCCGGUCCGUGCGGCGGCUGACCCGGCGGCGGGCCCACCGGCUGCUGCGGGCCCGGCGGCUGCUGAAGCG |
| | | GGAGGGCGUGCUGCAGGCCGCCGACUUCGACGAGAACGGCCUGAUCAAGUCCCUGCCCAACACCCCCUGGCAGCUGCGGGCCGCCGCCCUG |
| | | GACCGGAAGCUGACCCCCCUGGAGUGGUCCGCCGUGCUGCUGCACCUGAUCAAGCACCGGGGCUACCUGUCCCAGCGGAAGAACGAGGGCG |
| | | AGACCGCCGACAAGGAGCUGGGCGCCCUGCUGAAGGGCGUGGCCAACAACGCCCACGCCCUGCAGACCGGCGACUUCCGGACCCCCGCCGA |
| | | GCUGGCCCUGAACAAGUUCGAGAAGGAGUCCGGCCACAUCCGGAACCAGCGGGGCGACUACUCCCACACCUUCUCCCGGAAGGACCUGCAG |
| | | GCCGAGCUGAUCCUGCUGUUCGAGAAGCAGAAGGAGUUCGGCAACCCCCACGUGUCCGGCGGCCUGAAGGAGGGCAUCGAGACCCUGCUGA |
| | | UGACCCAGCGGCCCGCCCUGUCCGGCGACGCCGUGCAGAAGAUGCUGGGCCACUGCACCUUCGAGCCCGCCGAGCCCAAGGCCGCCAAGAA |
| | | CACCUACACCGCCGAGCGGUUCAUCUGGCUGACCAAGCUGAACAACCUGCGGAUCCUGGAGCAGGGCUCCGAGCGGCCCCUGACCGACACC |
| | | GAGCGGGCCACCCUGAUGGACGAGCCCUACCGGAAGUCCAAGCUGACCUACGCCCAGGCCCGGAAGCUGCUGGGCCUGGAGGACACCGCCU |
| | | UCUUCAAGGGCCUGCGGUACGGCAAGGACAACGCCGAGGCCUCCACCCUGAUGGAGAUGAAGGCCUACCACGCCAUCUCCCGGGCCCUGGA |
| | | GAAGGAGGGCCUGAAGGACAAGAAGUCCCCCCUGAACCUGUCCUCCGAGCUGCAGGACGAGAUCGGCACCGCCUUCUCCCUGUUCAAGACC |
| | | GACGAGGACAUCACCGGCCGGCUGAAGGACCGGGUGCAGCCCGAGAUCCUGGAGGCCCUGCUGAAGCACAUCUCCUUCGACAAGUUCGUGC |
| | | AGAUCUCCCUGAAGGCCCUGCGGCGGAUCGUGCCCCUGAUGGAGCAGGGCAAGCGGUACGACGAGGCCUGCGCCGAGAUCUACGGCGACCA |
| | | CUACGGCAAGAAGAACACCGAGGAGAAGAUCUACCUGCCCCCCAUCCCCGCCGACGAGAUCCGGAACCCCGUGGUGCUGCGGGCCCUGUCC |
| | | CAGGCCCGGAAGGUGAUCAACGGCGUGGUGCGGCGGUACGGCUCCCCCGCCCGGAUCCACAUCGAGACCGCCCGGGAGGUGGGCAAGUCCU |
| | | UCAAGGACCGGAAGGAGAUCGAGAAGCGGCAGGAGGAGAACCGGAAGGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCUGUACGAGCAGCAGCACGGCAAGUGCCUGUACUCCGGCAAGGAGAUC |
| | | AACCUGGUGCGGCUGAACGAGAAGGGCUACGUGGAGAUCGACCACGCCCUGCCCUUCUCCCGGACCUGGGACGACUCCUUCAACAACAAGG |
| | | UGCUGGUGCUGGGCUCCGAGAACCAGAACAAGGGCAACCAGACCCCCUACGAGUACUUCAACGGCAAGGACAACUCCCGGGAGUGGCAGGA |
| | | GUUCAAGGCCCGGGUGGAGACCUCCCGGUUCCCCCGGUCCAAGAAGCAGCGGAUCCUGCUGCAGAAGUUCGACGAGGACGGCUUCAAGGAG |
| | | UGCAACCUGAACGACACCCGGUACGUGAACCGCUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCACCACGCCCU |
| | | GGACGCCGUGGUGGUGGCCUGCUCCACCGUGGCCAUGCAGCAGAAGAUCACCCGGUUCGUGCGGUACAAGGAGAUGAACGCCUUCGACGGC |
| | | AAGACCAUCGACAAGGAGACCGGCAAGGUGCUGCACCAGAAGACCCACUUCCCCCAGCCCUGGGAGUUCUUCGCCCAGGAGGUGAUGAUCC |
| | | GGGUGUUCGGCAAGCCCGACGGCAAGCCCGAGUUCGAGGAGGCCGACACCCCCGAGAAGCUGCGGACCCUGCUGGCCGAGAAGCUGUCCUC |
| | | CCGGCCCGAGGCCGUGCACGAGUACGUGACCCCCCUGUUCGUGUCCCGGGCCCCCAACCGGAAGAUGUCCGGCGCCCACAAGGACACCCUG |
| | | CGGUCCGCCAAGCGGUUCGUGAAGCACAACGAGAAGAUCUCCGUGAAGCGGGUGUGGCUGACCGAGAUCAAGCUGGCCGACCUGGAGAACA |
| | | UGGUGAACUACAAGAACGGCCGGGAGAUCGAGCUGUACGAGGCCCUGAAGGCCCGGCUGGAGGCCUACGGCGGCAACGCCAAGCAGGCCUU |
| | | CGACCCCAAGGACAACCCCUUCUACAAGAAGGGCGGCCAGCUGGUGAAGGCCGUGCGGGUGGAGAAGACCCAGGAGUCCGGCGUGCUGCUG |
| | | AACAAGAAGAACGCCUACACCAUCGCCGACAACGGCGACAUGGUGCGGGUGGACGUGUUCUGCAAGGUGGACAAGAAGGGCAAGAACCAGU |
| | | ACUUCAUCGUGCCCAUCUACGCCUGGCAGGUGGCCGAGAACAUCCUGCCCGACAUCGACUGCAAGGGCUACCGGAUCGACGACUCCUACAC |
| | | CUUCUGCUUCUCCCUGCACAAGUACGACCUGAUCGCCUUCCAGAAGGACGAGAAGUCCAAGGUGGAGUUCGCCUACUACAUCAACUGCGAC |
| | | UCCUCCAACGGCCGGUUCUACCUGGCCUGGCACGACAAGGGCUCCAAGGAGCAGCAGUUCCGGAUCUCCACCCAGAACCUGGUGCUGAUCC |
| | | AGAAGUACCAGGUGAACGAGCUGGGCAAGGAGAUCCGGCCCUGCCGGCUGAAGAAGCGGCCCCCCGUGCGGUCCGGAAAGCGGACCGCCGA |
| | | CGGCUCCGGAGGAGGAAGCCCGAAGAAGAAGAGAAAGGUCAGCGAAAGCGCAACACCGGAAAGCGUCAGCGGAUGGAGACUGUUCAAGAAG |
| | | AUCAGCUAG |
| 55 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 61 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | P)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATESGGTSTESEGSAGTSTESEGSAGSAGSTSTEEGTSTESEGSAGTSTESEGSSSGAAFKPNPINYILGLDIGIASVGWAMV |
| | | EIDEEENPIRLIDLGVRVFERAEVPKTGDSLAMARRLARSVRRLTRRRAHRLLRARRLLKREGVLQAADFDENGLIKSLPNTPWQLRAAAL |
| | | DRKLTPLEWSAVLLHLIKHRGYLSQRKNEGETADKELGALLKGVANNAHALQTGDFRTPAELALNKFEKESGHIRNQRGDYSHTFSRKDLQ |
| | | AELILLFEKQKEFGNPHVSGGLKEGIETLLMTQRPALSGDAVQKMLGHCTFEPAEPKAAKNTYTAERFIWLTKLNNLRILEQGSERPLTDT |
| | | ERATLMDEPYRKSKLTYAQARKLLGLEDTAFFKGLRYGKDNAEASTLMEMKAYHAISRALEKEGLKDKKSPLNLSSELQDEIGTAFSLFKT |
| | | DEDITGRLKDRVQPEILEALLKHISFDKFVQISLKALRRIVPLMEQGKRYDEACAEIYGDHYGKKNTEEKIYLPPIPADEIRNPVVLRALS |
| | | QARKVINGVVRRYGSPARIHIETAREVGKSFKDRKEIEKRQEENRKDREKAAAKFREYFPNFVGEPKSKDILKLRLYEQQHGKCLYSGKEI |
| | | NLVRLNEKGYVEIDHALPFSRTWDDSFNNKVLVLGSENQNKGNQTPYEYFNGKDNSREWQEFKARVETSRFPRSKKQRILLQKFDEDGFKE |
| | | CNLNDTRYVNRFLCQFVADHILLTGKGKRRVFASNGQITNLLRGFWGLRKVRAENDRHHALDAVVVACSTVAMQQKITRFVRYKEMNAFDG |
| | | KTIDKETGKVLHQKTHFPQPWEFFAQEVMIRVFGKPDGKPEFEEADTPEKLRTLLAEKLSSRPEAVHEYVTPLFVSRAPNRKMSGAHKDTL |
| | | RSAKRFVKHNEKISVKRVWLTEIKLADLENMVNYKNGREIELYEALKARLEAYGGNAKQAFDPKDNPFYKKGGQLVKAVRVEKTQESGVLL |
| | | NKKNAYTIADNGDMVRVDVFCKVDKKGKNQYFIVPIYAWQVAENILPDIDCKGYRIDDSYTFCFSLHKYDLIAFQKDEKSKVEFAYYINCD |
| | | SSNGRFYLAWHDKGSKEQQFRISTQNLVLIQKYQVNELGKEIRPCRLKKRPPVRSGKRTADGSGGGSPKKKRKVSESATPESVSGWRLFKK |
| | | IS* |
| 56 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 61 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | P)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCGCCGGCACCUCCACCGAGUCCGAGGG |
| | | CUCCUCCUCCGGUGCCGCCUUCAAGCCCAACCCCAUCAACUACAUCCUGGGCCUGGACAUCGGCAUCGCCUCCGUGGGCUGGGCCAUGGUG |
| | | GAGAUCGACGAGGAGGAGAACCCCAUCCGGCUGAUCGACCUGGGCGUGCGGGUGUUCGAGCGGGCCGAGGUGCCCAAGACCGGCGACUCCC |
| | | UGGCCAUGGCCCGGCGGCUGGCCCGGUCCGUGCGGCGGCUGACCCGGGGGGGGGCCCACCGGCUGCUGCGGGCCCGGCGGCUGCUGAAGCG |
| | | GGAGGGCGUGCUGCAGGCCGCCGACUUCGACGAGAACGGCCUGAUCAAGUCCCUGCCCAACACCCCCUGGCAGCUGCGGGCCGCCGCCCUG |
| | | GACCGGAAGCUGACCCCCCUGGAGUGGUCCGCCGUGCUGCUGCACCUGAUCAAGCACCGGGGCUACCUGUCCCAGCGGAAGAACGAGGGCG |
| | | AGACCGCCGACAAGGAGCUGGGCGCCCUGCUGAAGGGCGUGGCCAACAACGCCCACGCCCUGCAGACCGGCGACUUCCGGACCCCCGCCGA |
| | | GCUGGCCCUGAACAAGUUCGAGAAGGAGUCCGGCCACAUCCGGAACCAGCGGGGCGACUACUCCCACACCUUCUCCCGGAAGGACCUGCAG |
| | | GCCGAGCUGAUCCUGCUGUUCGAGAAGCAGAAGGAGUUCGGCAACCCCCACGUGUCCGGCGGCCUGAAGGAGGGCAUCGAGACCCUGCUGA |
| | | UGACCCAGCGGCCCGCCCUGUCCGGCGACGCCGUGCAGAAGAUGCUGGGCCACUGCACCUUCGAGCCCGCCGAGCCCAAGGCCGCCAAGAA |
| | | CACCUACACCGCCGAGCGGUUCAUCUGGCUGACCAAGCUGAACAACCUGCGGAUCCUGGAGCAGGGCUCCGAGCGGCCCCUGACCGACACC |
| | | GAGCGGGCCACCCUGAUGGACGAGCCCUACCGGAAGUCCAAGCUGACCUACGCCCAGGCCCGGAAGCUGCUGGGCCUGGAGGACACCGCCU |
| | | UCUUCAAGGGCCUGCGGUACGGCAAGGACAACGCCGAGGCCUCCACCCUGAUGGAGAUGAAGGCCUACCACGCCAUCUCCCGGGCCCUGGA |
| | | GAAGGAGGGCCUGAAGGACAAGAAGUCCCCCCUGAACCUGUCCUCCGAGCUGCAGGACGAGAUCGGCACCGCCUUCUCCCUGUUCAAGACC |
| | | GACGAGGACAUCACCGGCCGGCUGAAGGACCGGGUGCAGCCCGAGAUCCUGGAGGCCCUGCUGAAGCACAUCUCCUUCGACAAGUUCGUGC |
| | | AGAUCUCCCUGAAGGCCCUGCGGCGGAUCGUGCCCCUGAUGGAGCAGGGCAAGCGGUACGACGAGGCCUGCGCCGAGAUCUACGGCGACCA |
| | | CUACGGCAAGAAGAACACCGAGGAGAAGAUCUACCUGCCCCCCAUCCCCGCCGACGAGAUCCGGAACCCCGUGGUGCUGCGGGCCCUGUCC |
| | | CAGGCCCGGAAGGUGAUCAACGGCGUGGUGCGGCGGUACGGCUCCCCCGCCCGGAUCCACAUCGAGACCGCCCGGGAGGUGGGCAAGUCCU |
| | | UCAAGGACCGGAAGGAGAUCGAGAAGCGGCAGGAGGAGAACCGGAAGGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCUGUACGAGCAGCAGCACGGCAAGUGCCUGUACUCCGGCAAGGAGAUC |
| | | AACCUGGUGCGGCUGAACGAGAAGGGCUACGUGGAGAUCGACCACGCCCUGCCCUUCUCCCGGACCUGGGACGACUCCUUCAACAACAAGG |
| | | UGCUGGUGCUGGGCUCCGAGAACCAGAACAAGGGCAACCAGACCCCCUACGAGUACUUCAACGGCAAGGACAACUCCCGGGAGUGGCAGGA |
| | | GUUCAAGGCCCGGGUGGAGACCUCCCGGUUCCCCCGGUCCAAGAAGCAGCGGAUCCUGCUGCAGAAGUUCGACGAGGACGGCUUCAAGGAG |
| | | UGCAACCUGAACGACACCCGGUACGUGAACCGCUUCCUGUGCCAGUUCGUGGCCGACCACAUCCUGCUGACCGGCAAGGGCAAGCGGCGGG |
| | | UGUUCGCCUCCAACGGCCAGAUCACCAACCUGCUGCGGGGCUUCUGGGGCCUGCGGAAGGUGCGGGCCGAGAACGACCGGCACCACGCCCU |
| | | GGACGCCGUGGUGGUGGCCUGCUCCACCGUGGCCAUGCAGCAGAAGAUCACCCGGUUCGUGCGGUACAAGGAGAUGAACGCCUUCGACGGC |
| | | AAGACCAUCGACAAGGAGACCGGCAAGGUGCUGCACCAGAAGACCCACUUCCCCCAGCCCUGGGAGUUCUUCGCCCAGGAGGUGAUGAUCC |
| | | GGGUGUUCGGCAAGCCCGACGGCAAGCCCGAGUUCGAGGAGGCCGACACCCCCGAGAAGCUGCGGACCCUGCUGGCCGAGAAGCUGUCCUC |
| | | CCGGCCCGAGGCCGUGCACGAGUACGUGACCCCCCUGUUCGUGUCCCGGGCCCCCAACCGGAAGAUGUCCGGCGCCCACAAGGACACCCUG |
| | | CGGUCCGCCAAGCGGUUCGUGAAGCACAACGAGAAGAUCUCCGUGAAGCGGGUGUGGCUGACCGAGAUCAAGCUGGCCGACCUGGAGAACA |
| | | UGGUGAACUACAAGAACGGCCGGGAGAUCGAGCUGUACGAGGCCCUGAAGGCCCGGCUGGAGGCCUACGGCGGCAACGCCAAGCAGGCCUU |
| | | CGACCCCAAGGACAACCCCUUCUACAAGAAGGGCGGCCAGCUGGUGAAGGCCGUGCGGGUGGAGAAGACCCAGGAGUCCGGCGUGCUGCUG |
| | | AACAAGAAGAACGCCUACACCAUCGCCGACAACGGCGACAUGGUGCGGGUGGACGUGUUCUGCAAGGUGGACAAGAAGGGCAAGAACCAGU |
| | | ACUUCAUCGUGCCCAUCUACGCCUGGCAGGUGGCCGAGAACAUCCUGCCCGACAUCGACUGCAAGGGCUACCGGAUCGACGACUCCUACAC |
| | | CUUCUGCUUCUCCCUGCACAAGUACGACCUGAUCGCCUUCCAGAAGGACGAGAAGUCCAAGGUGGAGUUCGCCUACUACAUCAACUGCGAC |
| | | UCCUCCAACGGCCGGUUCUACCUGGCCUGGCACGACAAGGGCUCCAAGGAGCAGCAGUUCCGGAUCUCCACCCAGAACCUGGUGCUGAUCC |
| | | AGAAGUACCAGGUGAACGAGCUGGGCAAGGAGAUCCGGCCCUGCCGGCUGAAGAAGCGGCCCCCCGUGCGGUCCGGAAAGCGGACCGCCGA |
| | | CGGCUCCGGAGGAGGAAGCCCGAAGAAGAAGAGAAAGGUCUAG |
| 57 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 61 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | P)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | amino | LDKAGFIKRQLVETRQITKHVAQILDSRMNTKYDENDKLIREVKVITLKSKLVSDFRKDFQFYKVREINNYHHAHDAYLNAVVGTALIKKY |
| | acid | PKLESEFVYGDYKVYDVRKMIAKSEQEIGKATAKYFFYSNIMNFFKTEITLANGEIRKRPLIETNGETGEIVWDKGRDFATVRKVLSMPQV |
| | sequence | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSSSGAAFKPNPINYILGLDIGIASVGWAMV |
| | | EIDEEENPIRLIDLGVRVFERAEVPKTGDSLAMARRLARSVRRLTRRRAHRLLRARRLLKREGVLQAADFDENGLIKSLPNTPWQLRAAAL |
| | | DRKLTPLEWSAVLLHLIKHRGYLSQRKNEGETADKELGALLKGVANNAHALQTGDFRTPAELALNKFEKESGHIRNQRGDYSHTFSRKDLQ |
| | | AELILLFEKQKEFGNPHVSGGLKEGIETLLMTQRPALSGDAVQKMLGHCTFEPAEPKAAKNTYTAERFIWLTKLNNLRILEQGSERPLTDT |
| | | ERATLMDEPYRKSKLTYAQARKLLGLEDTAFFKGLRYGKDNAEASTLMEMKAYHAISRALEKEGLKDKKSPLNLSSELQDEIGTAFSLFKT |
| | | DEDITGRLKDRVQPEILEALLKHISFDKFVQISLKALRRIVPLMEQGKRYDEACAEIYGDHYGKKNTEEKIYLPPIPADEIRNPVVLRALS |
| | | QARKVINGVVRRYGSPARIHIETAREVGKSFKDRKEIEKRQEENRKDREKAAAKFREYFPNFVGEPKSKDILKLRLYEQQHGKCLYSGKEI |
| | | NLVRLNEKGYVEIDHALPFSRTWDDSFNNKVLVLGSENQNKGNQTPYEYFNGKDNSREWQEFKARVETSRFPRSKKQRILLQKFDEDGFKE |
| | | CNLNDTRYVNRFLCQFVADHILLTGKGKRRVFASNGQITNLLRGFWGLRKVRAENDRHHALDAVVVACSTVAMQQKITRFVRYKEMNAFDG |
| | | KTIDKETGKVLHQKTHFPQPWEFFAQEVMIRVFGKPDGKPEFEEADTPEKLRTLLAEKLSSRPEAVHEYVTPLFVSRAPNRKMSGAHKDTL |
| | | RSAKRFVKHNEKISVKRVWLTEIKLADLENMVNYKNGREIELYEALKARLEAYGGNAKQAFDPKDNPFYKKGGQLVKAVRVEKTQESGVLL |
| | | NKKNAYTIADNGDMVRVDVFCKVDKKGKNQYFIVPIYAWQVAENILPDIDCKGYRIDDSYTFCFSLHKYDLIAFQKDEKSKVEFAYYINCD |
| | | SSNGRFYLAWHDKGSKEQQFRISTQNLVLIQKYQVNELGKEIRPCRLKKRPPVRSGKRTADGSGGGSPKKKRKV* |
| 58 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 51 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | Q)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCACCGAGGAGGGCACCUCC |
| | | ACCGAGUCCGAGGGCUCCUCCUCCGGUGCCGCCUUCAAGCCCAACCCCAUCAACUACAUCCUGGGCCUGGACAUCGGCAUCGCCUCCGUGG |
| | | GCUGGGCCAUGGUGGAGAUCGACGAGGAGGAGAACCCCAUCCGGCUGAUCGACCUGGGCGUGCGGGUGUUCGAGCGGGCCGAGGUGCCCAA |
| | | GACCGGCGACUCCCUGGCCAUGGCCCGGCGGCUGGCCCGGUCCGUGCGGCGGCUGACCCGGGGGGGGGCCCACCGGCUGCUGCGGGCCCGG |
| | | CGGCUGCUGAAGCGGGAGGGCGUGCUGCAGGCCGCCGACUUCGACGAGAACGGCCUGAUCAAGUCCCUGCCCAACACCCCCUGGCAGCUGC |
| | | GGGCCGCCGCCCUGGACCGGAAGCUGACCCCCCUGGAGUGGUCCGCCGUGCUGCUGCACCUGAUCAAGCACCGGGGCUACCUGUCCCAGCG |
| | | GAAGAACGAGGGCGAGACCGCCGACAAGGAGCUGGGCGCCCUGCUGAAGGGCGUGGCCAACAACGCCCACGCCCUGCAGACCGGCGACUUC |
| | | CGGACCCCCGCCGAGCUGGCCCUGAACAAGUUCGAGAAGGAGUCCGGCCACAUCCGGAACCAGCGGGGCGACUACUCCCACACCUUCUCCC |
| | | GGAAGGACCUGCAGGCCGAGCUGAUCCUGCUGUUCGAGAAGCAGAAGGAGUUCGGCAACCCCCACGUGUCCGGCGGCCUGAAGGAGGGCAU |
| | | CGAGACCCUGCUGAUGACCCAGCGGCCCGCCCUGUCCGGCGACGCCGUGCAGAAGAUGCUGGGCCACUGCACCUUCGAGCCCGCCGAGCCC |
| | | AAGGCCGCCAAGAACACCUACACCGCCGAGCGGUUCAUCUGGCUGACCAAGCUGAACAACCUGCGGAUCCUGGAGCAGGGCUCCGAGCGGC |
| | | CCCUGACCGACACCGAGCGGGCCACCCUGAUGGACGAGCCCUACCGGAAGUCCAAGCUGACCUACGCCCAGGCCCGGAAGCUGCUGGGCCU |
| | | GGAGGACACCGCCUUCUUCAAGGGCCUGCGGUACGGCAAGGACAACGCCGAGGCCUCCACCCUGAUGGAGAUGAAGGCCUACCACGCCAUC |
| | | UCCCGGGCCCUGGAGAAGGAGGGCCUGAAGGACAAGAAGUCCCCCCUGAACCUGUCCUCCGAGCUGCAGGACGAGAUCGGCACCGCCUUCU |
| | | CCCUGUUCAAGACCGACGAGGACAUCACCGGCCGGCUGAAGGACCGGGUGCAGCCCGAGAUCCUGGAGGCCCUGCUGAAGCACAUCUCCUU |
| | | CGACAAGUUCGUGCAGAUCUCCCUGAAGGCCCUGCGGCGGAUCGUGCCCCUGAUGGAGCAGGGCAAGCGGUACGACGAGGCCUGCGCCGAG |
| | | AUCUACGGCGACCACUACGGCAAGAAGAACACCGAGGAGAAGAUCUACCUGCCCCCCAUCCCCGCCGACGAGAUCCGGAACCCCGUGGUGC |
| | | UGCGGGCCCUGUCCCAGGCCCGGAAGGUGAUCAACGGCGUGGUGCGGCGGUACGGCUCCCCCGCCCGGAUCCACAUCGAGACCGCCCGGGA |
| | | GGUGGGCAAGUCCUUCAAGGACCGGAAGGAGAUCGAGAAGCGGCAGGAGGAGAACCGGAAGGACCGGGAGAAGGCCGCCGCCAAGUUCCGG |
| | | GAGUACUUCCCCAACUUCGUGGGCGAGCCCAAGUCCAAGGACAUCCUGAAGCUGCGGCUGUACGAGCAGCAGCACGGCAAGUGCCUGUACU |
| | | CCGGCAAGGAGAUCAACCUGGUGCGGCUGAACGAGAAGGGCUACGUGGAGAUCGACCACGCCCUGCCCUUCUCCCGGACCUGGGACGACUC |
| | | CUUCAACAACAAGGUGCUGGUGCUGGGCUCCGAGAACCAGAACAAGGGCAACCAGACCCCCUACGAGUACUUCAACGGCAAGGACAACUCC |
| | | CGGGAGUGGCAGGAGUUCAAGGCCCGGGUGGAGACCUCCCGGUUCCCCCGGUCCAAGAAGCAGCGGAUCCUGCUGCAGAAGUUCGACGAGG |
| | | ACGGCUUCAAGGAGUGCAACCUGAACGACACCCGGUACGUGAACCGCUUCCUGUGCCAGUUCGUGGCCGACCACAUCCUGCUGACCGGCAA |
| | | GGGCAAGCGGCGGGUGUUCGCCUCCAACGGCCAGAUCACCAACCUGCUGCGGGGCUUCUGGGGCCUGCGGAAGGUGCGGGCCGAGAACGAC |
| | | CGGCACCACGCCCUGGACGCCGUGGUGGUGGCCUGCUCCACCGUGGCCAUGCAGCAGAAGAUCACCCGGUUCGUGCGGUACAAGGAGAUGA |
| | | ACGCCUUCGACGGCAAGACCAUCGACAAGGAGACCGGCAAGGUGCUGCACCAGAAGACCCACUUCCCCCAGCCCUGGGAGUUCUUCGCCCA |
| | | GGAGGUGAUGAUCCGGGUGUUCGGCAAGCCCGACGGCAAGCCCGAGUUCGAGGAGGCCGACACCCCCGAGAAGCUGCGGACCCUGCUGGCC |
| | | GAGAAGCUGUCCUCCCGGCCCGAGGCCGUGCACGAGUACGUGACCCCCCUGUUCGUGUCCCGGGCCCCCAACCGGAAGAUGUCCGGCGCCC |
| | | ACAAGGACACCCUGCGGUCCGCCAAGCGGUUCGUGAAGCACAACGAGAAGAUCUCCGUGAAGCGGGUGUGGCUGACCGAGAUCAAGCUGGC |
| | | CGACCUGGAGAACAUGGUGAACUACAAGAACGGCCGGGAGAUCGAGCUGUACGAGGCCCUGAAGGCCCGGCUGGAGGCCUACGGCGGCAAC |
| | | GCCAAGCAGGCCUUCGACCCCAAGGACAACCCCUUCUACAAGAAGGGCGGCCAGCUGGUGAAGGCCGUGCGGGUGGAGAAGACCCAGGAGU |
| | | CCGGCGUGCUGCUGAACAAGAAGAACGCCUACACCAUCGCCGACAACGGCGACAUGGUGCGGGUGGACGUGUUCUGCAAGGUGGACAAGAA |
| | | GGGCAAGAACCAGUACUUCAUCGUGCCCAUCUACGCCUGGCAGGUGGCCGAGAACAUCCUGCCCGACAUCGACUGCAAGGGCUACCGGAUC |
| | | GACGACUCCUACACCUUCUGCUUCUCCCUGCACAAGUACGACCUGAUCGCCUUCCAGAAGGACGAGAAGUCCAAGGUGGAGUUCGCCUACU |
| | | ACAUCAACUGCGACUCCUCCAACGGCCGGUUCUACCUGGCCUGGCACGACAAGGGCUCCAAGGAGCAGCAGUUCCGGAUCUCCACCCAGAA |
| | | CCUGGUGCUGAUCCAGAAGUACCAGGUGAACGAGCUGGGCAAGGAGAUCCGGCCCUGCCGGCUGAAGAAGCGGCCCCCCGUGCGGUCCGGA |
| | | AAGCGGACCGCCGACGGCUCCGGAGGAGGAAGCCCGAAGAAGAAGAGAAAGGUCAGCGAAAGCGCAACACCGGAAAGCGUCAGCGGAUGGA |
| | | GACUGUUCAAGAAGAUCAGCUAGCUAGCACCAGCCUCAAGAACACCCGAAUGGAGUCUCUAAGCUACAUAAUACCAACUUACACUUUACAA |
| | | AAUGUUGUCCCCCAAAAUGUAGCCAUUCGUAUCUGCUCCUAAUAAAAAGAAAGUUUCUUCACAUUCUCUCGAGAAAAAAAAAAAAUGGAAA |
| | | AAAAAAAAACGGAAAAAAAAAAAAGGUAAAAAAAAAAAAUAUAAAAAAAAAAAACAUAAAAAAAAAAAACGAAAAAAAAAAAACGUAAAAA |
| | | AAAAAAACUCAAAAAAAAAAAAGAUAAAAAAAAAAAACCUAAAAAAAAAAAAUGUAAAAAAAAAAAAGGGAAAAAAAAAAAACGCAAAAAA |
| | | AAAAAACACAAAAAAAAAAAAUGCAAAAAAAAAAAAUCGAAAAAAAAAAAAUCUAAAAAAAAAAAACGAAAAAAAAAAAACCCAAAAAAAA |
| | | AAAAGACAAAAAAAAAAAAUAGAAAAAAAAAAAAGUUAAAAAAAAAAAACUGAAAAAAAAAAAAUUUAAAAAAAAAAAAUCUAG |
| 59 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 51 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | Q)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCUCCUCCGGUGCCGCCUUCAAGCCCAA |
| | | CCCCAUCAACUACAUCCUGGGCCUGGACAUCGGCAUCGCCUCCGUGGGCUGGGCCAUGGUGGAGAUCGACGAGGAGGAGAACCCCAUCCGG |
| | | CUGAUCGACCUGGGCGUGCGGGUGUUCGAGCGGGCCGAGGUGCCCAAGACCGGCGACUCCCUGGCCAUGGCCCGGCGGCUGGCCCGGUCCG |
| | | UGCGGCGGCUGACCCGGCGGCGGGCCCACCGGCUGCUGCGGGCCCGGCGGCUGCUGAAGCGGGAGGGCGUGCUGCAGGCCGCCGACUUCGA |
| | | CGAGAACGGCCUGAUCAAGUCCCUGCCCAACACCCCCUGGCAGCUGCGGGCCGCCGCCCUGGACCGGAAGCUGACCCCCCUGGAGUGGUCC |
| | | GCCGUGCUGCUGCACCUGAUCAAGCACCGGGGCUACCUGUCCCAGCGGAAGAACGAGGGCGAGACCGCCGACAAGGAGCUGGGCGCCCUGC |
| | | UGAAGGGCGUGGCCAACAACGCCCACGCCCUGCAGACCGGCGACUUCCGGACCCCCGCCGAGCUGGCCCUGAACAAGUUCGAGAAGGAGUC |
| | | CGGCCACAUCCGGAACCAGCGGGGCGACUACUCCCACACCUUCUCCCGGAAGGACCUGCAGGCCGAGCUGAUCCUGCUGUUCGAGAAGCAG |
| | | AAGGAGUUCGGCAACCCCCACGUGUCCGGCGGCCUGAAGGAGGGCAUCGAGACCCUGCUGAUGACCCAGCGGCCCGCCCUGUCCGGCGACG |
| | | CCGUGCAGAAGAUGCUGGGCCACUGCACCUUCGAGCCCGCCGAGCCCAAGGCCGCCAAGAACACCUACACCGCCGAGCGGUUCAUCUGGCU |
| | | GACCAAGCUGAACAACCUGCGGAUCCUGGAGCAGGGCUCCGAGCGGCCCCUGACCGACACCGAGCGGGCCACCCUGAUGGACGAGCCCUAC |
| | | CGGAAGUCCAAGCUGACCUACGCCCAGGCCCGGAAGCUGCUGGGCCUGGAGGACACCGCCUUCUUCAAGGGCCUGCGGUACGGCAAGGACA |
| | | ACGCCGAGGCCUCCACCCUGAUGGAGAUGAAGGCCUACCACGCCAUCUCCCGGGCCCUGGAGAAGGAGGGCCUGAAGGACAAGAAGUCCCC |
| | | CCUGAACCUGUCCUCCGAGCUGCAGGACGAGAUCGGCACCGCCUUCUCCCUGUUCAAGACCGACGAGGACAUCACCGGCCGGCUGAAGGAC |
| | | CGGGUGCAGCCCGAGAUCCUGGAGGCCCUGCUGAAGCACAUCUCCUUCGACAAGUUCGUGCAGAUCUCCCUGAAGGCCCUGCGGCGGAUCG |
| | | UGCCCCUGAUGGAGCAGGGCAAGCGGUACGACGAGGCCUGCGCCGAGAUCUACGGCGACCACUACGGCAAGAAGAACACCGAGGAGAAGAU |
| | | CUACCUGCCCCCCAUCCCCGCCGACGAGAUCCGGAACCCCGUGGUGCUGCGGGCCCUGUCCCAGGCCCGGAAGGUGAUCAACGGCGUGGUG |
| | | CGGCGGUACGGCUCCCCCGCCCGGAUCCACAUCGAGACCGCCCGGGAGGUGGGCAAGUCCUUCAAGGACCGGAAGGAGAUCGAGAAGCGGC |
| | | AGGAGGAGAACCGGAAGGACCGGGAGAAGGCCGCCGCCAAGUUCCGGGAGUACUUCCCCAACUUCGUGGGCGAGCCCAAGUCCAAGGACAU |
| | | CCUGAAGCUGCGGCUGUACGAGCAGCAGCACGGCAAGUGCCUGUACUCCGGCAAGGAGAUCAACCUGGUGCGGCUGAACGAGAAGGGCUAC |
| | | GUGGAGAUCGACCACGCCCUGCCCUUCUCCCGGACCUGGGACGACUCCUUCAACAACAAGGUGCUGGUGCUGGGCUCCGAGAACCAGAACA |
| | | AGGGCAACCAGACCCCCUACGAGUACUUCAACGGCAAGGACAACUCCCGGGAGUGGCAGGAGUUCAAGGCCCGGGUGGAGACCUCCCGGUU |
| | | CCCCCGGUCCAAGAAGCAGCGGAUCCUGCUGCAGAAGUUCGACGAGGACGGCUUCAAGGAGUGCAACCUGAACGACACCCGGUACGUGAAC |
| | | CGCUUCCUGUGCCAGUUCGUGGCCGACCACAUCCUGCUGACCGGCAAGGGCAAGCGGCGGGUGUUCGCCUCCAACGGCCAGAUCACCAACC |
| | | UGCUGCGGGGCUUCUGGGGCCUGCGGAAGGUGCGGGCCGAGAACGACCGGCACCACGCCCUGGACGCCGUGGUGGUGGCCUGCUCCACCGU |
| | | GGCCAUGCAGCAGAAGAUCACCCGGUUCGUGCGGUACAAGGAGAUGAACGCCUUCGACGGCAAGACCAUCGACAAGGAGACCGGCAAGGUG |
| | | CUGCACCAGAAGACCCACUUCCCCCAGCCCUGGGAGUUCUUCGCCCAGGAGGUGAUGAUCCGGGUGUUCGGCAAGCCCGACGGCAAGCCCG |
| | | AGUUCGAGGAGGCCGACACCCCCGAGAAGCUGCGGACCCUGCUGGCCGAGAAGCUGUCCUCCCGGCCCGAGGCCGUGCACGAGUACGUGAC |
| | | CCCCCUGUUCGUGUCCCGGGCCCCCAACCGGAAGAUGUCCGGCGCCCACAAGGACACCCUGCGGUCCGCCAAGCGGUUCGUGAAGCACAAC |
| | | GAGAAGAUCUCCGUGAAGCGGGUGUGGCUGACCGAGAUCAAGCUGGCCGACCUGGAGAACAUGGUGAACUACAAGAACGGCCGGGAGAUCG |
| | | AGCUGUACGAGGCCCUGAAGGCCCGGCUGGAGGCCUACGGCGGCAACGCCAAGCAGGCCUUCGACCCCAAGGACAACCCCUUCUACAAGAA |
| | | GGGCGGCCAGCUGGUGAAGGCCGUGCGGGUGGAGAAGACCCAGGAGUCCGGCGUGCUGCUGAACAAGAAGAACGCCUACACCAUCGCCGAC |
| | | AACGGCGACAUGGUGCGGGUGGACGUGUUCUGCAAGGUGGACAAGAAGGGCAAGAACCAGUACUUCAUCGUGCCCAUCUACGCCUGGCAGG |
| | | UGGCCGAGAACAUCCUGCCCGACAUCGACUGCAAGGGCUACCGGAUCGACGACUCCUACACCUUCUGCUUCUCCCUGCACAAGUACGACCU |
| | | GAUCGCCUUCCAGAAGGACGAGAAGUCCAAGGUGGAGUUCGCCUACUACAUCAACUGCGACUCCUCCAACGGCCGGUUCUACCUGGCCUGG |
| | | CACGACAAGGGCUCCAAGGAGCAGCAGUUCCGGAUCUCCACCCAGAACCUGGUGCUGAUCCAGAAGUACCAGGUGAACGAGCUGGGCAAGG |
| | | AGAUCCGGCCCUGCCGGCUGAAGAAGCGGCCCCCCGUGCGGUCCGGAAAGCGGACCGCCGACGGCUCCGGAGGAGGAAGCCCGAAGAAGAA |
| | | GAGAAAGGUCAGCGAAAGCGCAACACCGGAAAGCGUCAGCGGAUGGAGACUGUUCAAGAAGAUCAGCUAG |
| 60 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 51 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | Q)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATESGGTSTESEGSAGTSTESEGSAGSAGSTSTEEGTSTESEGSSSGAAFKPNPINYILGLDIGIASVGWAMVEIDEEENPIR |
| | | LIDLGVRVFERAEVPKTGDSLAMARRLARSVRRLTRRRAHRLLRARRLLKREGVLQAADFDENGLIKSLPNTPWQLRAAALDRKLTPLEWS |
| | | AVLLHLIKHRGYLSQRKNEGETADKELGALLKGVANNAHALQTGDFRTPAELALNKFEKESGHIRNQRGDYSHTFSRKDLQAELILLFEKQ |
| | | KEFGNPHVSGGLKEGIETLLMTQRPALSGDAVQKMLGHCTFEPAEPKAAKNTYTAERFIWLTKLNNLRILEQGSERPLTDTERATLMDEPY |
| | | RKSKLTYAQARKLLGLEDTAFFKGLRYGKDNAEASTLMEMKAYHAISRALEKEGLKDKKSPLNLSSELQDEIGTAFSLFKTDEDITGRLKD |
| | | RVQPEILEALLKHISFDKFVQISLKALRRIVPLMEQGKRYDEACAEIYGDHYGKKNTEEKIYLPPIPADEIRNPVVLRALSQARKVINGVV |
| | | RRYGSPARIHIETAREVGKSFKDRKEIEKRQEENRKDREKAAAKFREYFPNFVGEPKSKDILKLRLYEQQHGKCLYSGKEINLVRLNEKGY |
| | | VEIDHALPFSRTWDDSFNNKVLVLGSENQNKGNQTPYEYFNGKDNSREWQEFKARVETSRFPRSKKQRILLQKFDEDGFKECNLNDTRYVN |
| | | RFLCQFVADHILLTGKGKRRVFASNGQITNLLRGFWGLRKVRAENDRHHALDAVVVACSTVAMQQKITRFVRYKEMNAFDGKTIDKETGKV |
| | | LHQKTHFPQPWEFFAQEVMIRVFGKPDGKPEFEEADTPEKLRTLLAEKLSSRPEAVHEYVTPLFVSRAPNRKMSGAHKDTLRSAKRFVKHN |
| | | EKISVKRVWLTEIKLADLENMVNYKNGREIELYEALKARLEAYGGNAKQAFDPKDNPFYKKGGQLVKAVRVEKTQESGVLLNKKNAYTIAD |
| | | NGDMVRVDVFCKVDKKGKNQYFIVPIYAWQVAENILPDIDCKGYRIDDSYTFCFSLHKYDLIAFQKDEKSKVEFAYYINCDSSNGRFYLAW |
| | | HDKGSKEQQFRISTQNLVLIQKYQVNELGKEIRPCRLKKRPPVRSGKRTADGSGGGSPKKKRKVSESATPESVSGWRLFKKIS* |
| 61 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 51 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | Q)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCACCGAGGAGGGCACCUCCACCGAGUCCGAGGGCUCCUCCUCCGGUGCCGCCUUCAAGCCCAA |
| | | CCCCAUCAACUACAUCCUGGGCCUGGACAUCGGCAUCGCCUCCGUGGGCUGGGCCAUGGUGGAGAUCGACGAGGAGGAGAACCCCAUCCGG |
| | | CUGAUCGACCUGGGCGUGCGGGUGUUCGAGCGGGCCGAGGUGCCCAAGACCGGCGACUCCCUGGCCAUGGCCCGGCGGCUGGCCCGGUCCG |
| | | UGCGGCGGCUGACCCGGCGGCGGGCCCACCGGCUGCUGCGGGCCCGGCGGCUGCUGAAGCGGGAGGGCGUGCUGCAGGCCGCCGACUUCGA |
| | | CGAGAACGGCCUGAUCAAGUCCCUGCCCAACACCCCCUGGCAGCUGCGGGCCGCCGCCCUGGACCGGAAGCUGACCCCCCUGGAGUGGUCC |
| | | GCCGUGCUGCUGCACCUGAUCAAGCACCGGGGCUACCUGUCCCAGCGGAAGAACGAGGGCGAGACCGCCGACAAGGAGCUGGGCGCCCUGC |
| | | UGAAGGGCGUGGCCAACAACGCCCACGCCCUGCAGACCGGCGACUUCCGGACCCCCGCCGAGCUGGCCCUGAACAAGUUCGAGAAGGAGUC |
| | | CGGCCACAUCCGGAACCAGCGGGGCGACUACUCCCACACCUUCUCCCGGAAGGACCUGCAGGCCGAGCUGAUCCUGCUGUUCGAGAAGCAG |
| | | AAGGAGUUCGGCAACCCCCACGUGUCCGGCGGCCUGAAGGAGGGCAUCGAGACCCUGCUGAUGACCCAGCGGCCCGCCCUGUCCGGCGACG |
| | | CCGUGCAGAAGAUGCUGGGCCACUGCACCUUCGAGCCCGCCGAGCCCAAGGCCGCCAAGAACACCUACACCGCCGAGCGGUUCAUCUGGCU |
| | | GACCAAGCUGAACAACCUGCGGAUCCUGGAGCAGGGCUCCGAGCGGCCCCUGACCGACACCGAGCGGGCCACCCUGAUGGACGAGCCCUAC |
| | | CGGAAGUCCAAGCUGACCUACGCCCAGGCCCGGAAGCUGCUGGGCCUGGAGGACACCGCCUUCUUCAAGGGCCUGCGGUACGGCAAGGACA |
| | | ACGCCGAGGCCUCCACCCUGAUGGAGAUGAAGGCCUACCACGCCAUCUCCCGGGCCCUGGAGAAGGAGGGCCUGAAGGACAAGAAGUCCCC |
| | | CCUGAACCUGUCCUCCGAGCUGCAGGACGAGAUCGGCACCGCCUUCUCCCUGUUCAAGACCGACGAGGACAUCACCGGCCGGCUGAAGGAC |
| | | CGGGUGCAGCCCGAGAUCCUGGAGGCCCUGCUGAAGCACAUCUCCUUCGACAAGUUCGUGCAGAUCUCCCUGAAGGCCCUGCGGCGGAUCG |
| | | UGCCCCUGAUGGAGCAGGGCAAGCGGUACGACGAGGCCUGCGCCGAGAUCUACGGCGACCACUACGGCAAGAAGAACACCGAGGAGAAGAU |
| | | CUACCUGCCCCCCAUCCCCGCCGACGAGAUCCGGAACCCCGUGGUGCUGCGGGCCCUGUCCCAGGCCCGGAAGGUGAUCAACGGCGUGGUG |
| | | CGGCGGUACGGCUCCCCCGCCCGGAUCCACAUCGAGACCGCCCGGGAGGUGGGCAAGUCCUUCAAGGACCGGAAGGAGAUCGAGAAGCGGC |
| | | AGGAGGAGAACCGGAAGGACCGGGAGAAGGCCGCCGCCAAGUUCCGGGAGUACUUCCCCAACUUCGUGGGCGAGCCCAAGUCCAAGGACAU |
| | | CCUGAAGCUGCGGCUGUACGAGCAGCAGCACGGCAAGUGCCUGUACUCCGGCAAGGAGAUCAACCUGGUGCGGCUGAACGAGAAGGGCUAC |
| | | GUGGAGAUCGACCACGCCCUGCCCUUCUCCCGGACCUGGGACGACUCCUUCAACAACAAGGUGCUGGUGCUGGGCUCCGAGAACCAGAACA |
| | | AGGGCAACCAGACCCCCUACGAGUACUUCAACGGCAAGGACAACUCCCGGGAGUGGCAGGAGUUCAAGGCCCGGGUGGAGACCUCCCGGUU |
| | | CCCCCGGUCCAAGAAGCAGCGGAUCCUGCUGCAGAAGUUCGACGAGGACGGCUUCAAGGAGUGCAACCUGAACGACACCCGGUACGUGAAC |
| | | CGCUUCCUGUGCCAGUUCGUGGCCGACCACAUCCUGCUGACCGGCAAGGGCAAGCGGCGGGUGUUCGCCUCCAACGGCCAGAUCACCAACC |
| | | UGCUGCGGGGCUUCUGGGGCCUGCGGAAGGUGCGGGCCGAGAACGACCGGCACCACGCCCUGGACGCCGUGGUGGUGGCCUGCUCCACCGU |
| | | GGCCAUGCAGCAGAAGAUCACCCGGUUCGUGCGGUACAAGGAGAUGAACGCCUUCGACGGCAAGACCAUCGACAAGGAGACCGGCAAGGUG |
| | | CUGCACCAGAAGACCCACUUCCCCCAGCCCUGGGAGUUCUUCGCCCAGGAGGUGAUGAUCCGGGUGUUCGGCAAGCCCGACGGCAAGCCCG |
| | | AGUUCGAGGAGGCCGACACCCCCGAGAAGCUGCGGACCCUGCUGGCCGAGAAGCUGUCCUCCCGGCCCGAGGCCGUGCACGAGUACGUGAC |
| | | CCCCCUGUUCGUGUCCCGGGCCCCCAACCGGAAGAUGUCCGGCGCCCACAAGGACACCCUGCGGUCCGCCAAGCGGUUCGUGAAGCACAAC |
| | | GAGAAGAUCUCCGUGAAGCGGGUGUGGCUGACCGAGAUCAAGCUGGCCGACCUGGAGAACAUGGUGAACUACAAGAACGGCCGGGAGAUCG |
| | | AGCUGUACGAGGCCCUGAAGGCCCGGCUGGAGGCCUACGGCGGCAACGCCAAGCAGGCCUUCGACCCCAAGGACAACCCCUUCUACAAGAA |
| | | GGGCGGCCAGCUGGUGAAGGCCGUGCGGGUGGAGAAGACCCAGGAGUCCGGCGUGCUGCUGAACAAGAAGAACGCCUACACCAUCGCCGAC |
| | | AACGGCGACAUGGUGCGGGUGGACGUGUUCUGCAAGGUGGACAAGAAGGGCAAGAACCAGUACUUCAUCGUGCCCAUCUACGCCUGGCAGG |
| | | UGGCCGAGAACAUCCUGCCCGACAUCGACUGCAAGGGCUACCGGAUCGACGACUCCUACACCUUCUGCUUCUCCCUGCACAAGUACGACCU |
| | | GAUCGCCUUCCAGAAGGACGAGAAGUCCAAGGUGGAGUUCGCCUACUACAUCAACUGCGACUCCUCCAACGGCCGGUUCUACCUGGCCUGG |
| | | CACGACAAGGGCUCCAAGGAGCAGCAGUUCCGGAUCUCCACCCAGAACCUGGUGCUGAUCCAGAAGUACCAGGUGAACGAGCUGGGCAAGG |
| | | AGAUCCGGCCCUGCCGGCUGAAGAAGCGGCCCCCCGUGCGGUCCGGAAAGCGGACCGCCGACGGCUCCGGAGGAGGAAGCCCGAAGAAGAA |
| | | GAGAAAGGUCUAG |
| 62 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 51 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | Q)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | mRNA | LDKAGFIKRQLVETRQITKHVAQILDSRMNTKYDENDKLIREVKVITLKSKLVSDFRKDFQFYKVREINNYHHAHDAYLNAVVGTALIKKY |
| | sequence | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSSSGAAFKPNPINYILGLDIGIASVGWAMVEIDEEENPIR |
| | | LIDLGVRVFERAEVPKTGDSLAMARRLARSVRRLTRRRAHRLLRARRLLKREGVLQAADFDENGLIKSLPNTPWQLRAAALDRKLTPLEWS |
| | | AVLLHLIKHRGYLSQRKNEGETADKELGALLKGVANNAHALQTGDFRTPAELALNKFEKESGHIRNQRGDYSHTFSRKDLQAELILLFEKQ |
| | | KEFGNPHVSGGLKEGIETLLMTQRPALSGDAVQKMLGHCTFEPAEPKAAKNTYTAERFIWLTKLNNLRILEQGSERPLTDTERATLMDEPY |
| | | RKSKLTYAQARKLLGLEDTAFFKGLRYGKDNAEASTLMEMKAYHAISRALEKEGLKDKKSPLNLSSELQDEIGTAFSLFKTDEDITGRLKD |
| | | RVQPEILEALLKHISFDKFVQISLKALRRIVPLMEQGKRYDEACAEIYGDHYGKKNTEEKIYLPPIPADEIRNPVVLRALSQARKVINGVV |
| | | RRYGSPARIHIETAREVGKSFKDRKEIEKRQEENRKDREKAAAKFREYFPNFVGEPKSKDILKLRLYEQQHGKCLYSGKEINLVRLNEKGY |
| | | VEIDHALPFSRTWDDSFNNKVLVLGSENQNKGNQTPYEYFNGKDNSREWQEFKARVETSRFPRSKKQRILLQKFDEDGFKECNLNDTRYVN |
| | | RFLCQFVADHILLTGKGKRRVFASNGQITNLLRGFWGLRKVRAENDRHHALDAVVVACSTVAMQQKITRFVRYKEMNAFDGKTIDKETGKV |
| | | LHQKTHFPQPWEFFAQEVMIRVFGKPDGKPEFEEADTPEKLRTLLAEKLSSRPEAVHEYVTPLFVSRAPNRKMSGAHKDTLRSAKRFVKHN |
| | | EKISVKRVWLTEIKLADLENMVNYKNGREIELYEALKARLEAYGGNAKQAFDPKDNPFYKKGGQLVKAVRVEKTQESGVLLNKKNAYTIAD |
| | | NGDMVRVDVFCKVDKKGKNQYFIVPIYAWQVAENILPDIDCKGYRIDDSYTFCFSLHKYDLIAFQKDEKSKVEFAYYINCDSSNGRFYLAW |
| | | HDKGSKEQQFRISTQNLVLIQKYQVNELGKEIRPCRLKKRPPVRSGKRTADGSGGGSPKKKRKV* |
| 63 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 41 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | R)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCGCCGGCUCCGCCGGCUCCACCUCCGGCUCCUCCUCCGGUGCC |
| | | GCCUUCAAGCCCAACCCCAUCAACUACAUCCUGGGCCUGGACAUCGGCAUCGCCUCCGUGGGCUGGGCCAUGGUGGAGAUCGACGAGGAGG |
| | | AGAACCCCAUCCGGCUGAUCGACCUGGGCGUGCGGGUGUUCGAGCGGGCCGAGGUGCCCAAGACCGGCGACUCCCUGGCCAUGGCCCGGCG |
| | | GCUGGCCCGGUCCGUGCGGCGGCUGACCCGGCGGCGGGCCCACCGGCUGCUGCGGGCCCGGCGGCUGCUGAAGCGGGAGGGCGUGCUGCAG |
| | | GCCGCCGACUUCGACGAGAACGGCCUGAUCAAGUCCCUGCCCAACACCCCCUGGCAGCUGCGGGCCGCCGCCCUGGACCGGAAGCUGACCC |
| | | CCCUGGAGUGGUCCGCCGUGCUGCUGCACCUGAUCAAGCACCGGGGCUACCUGUCCCAGCGGAAGAACGAGGGCGAGACCGCCGACAAGGA |
| | | GCUGGGCGCCCUGCUGAAGGGCGUGGCCAACAACGCCCACGCCCUGCAGACCGGCGACUUCCGGACCCCCGCCGAGCUGGCCCUGAACAAG |
| | | UUCGAGAAGGAGUCCGGCCACAUCCGGAACCAGCGGGGCGACUACUCCCACACCUUCUCCCGGAAGGACCUGCAGGCCGAGCUGAUCCUGC |
| | | UGUUCGAGAAGCAGAAGGAGUUCGGCAACCCCCACGUGUCCGGCGGCCUGAAGGAGGGCAUCGAGACCCUGCUGAUGACCCAGCGGCCCGC |
| | | CCUGUCCGGCGACGCCGUGCAGAAGAUGCUGGGCCACUGCACCUUCGAGCCCGCCGAGCCCAAGGCCGCCAAGAACACCUACACCGCCGAG |
| | | CGGUUCAUCUGGCUGACCAAGCUGAACAACCUGCGGAUCCUGGAGCAGGGCUCCGAGCGGCCCCUGACCGACACCGAGCGGGCCACCCUGA |
| | | UGGACGAGCCCUACCGGAAGUCCAAGCUGACCUACGCCCAGGCCCGGAAGCUGCUGGGCCUGGAGGACACCGCCUUCUUCAAGGGCCUGCG |
| | | GUACGGCAAGGACAACGCCGAGGCCUCCACCCUGAUGGAGAUGAAGGCCUACCACGCCAUCUCCCGGGCCCUGGAGAAGGAGGGCCUGAAG |
| | | GACAAGAAGUCCCCCCUGAACCUGUCCUCCGAGCUGCAGGACGAGAUCGGCACCGCCUUCUCCCUGUUCAAGACCGACGAGGACAUCACCG |
| | | GCCGGCUGAAGGACCGGGUGCAGCCCGAGAUCCUGGAGGCCCUGCUGAAGCACAUCUCCUUCGACAAGUUCGUGCAGAUCUCCCUGAAGGC |
| | | CCUGCGGCGGAUCGUGCCCCUGAUGGAGCAGGGCAAGCGGUACGACGAGGCCUGCGCCGAGAUCUACGGCGACCACUACGGCAAGAAGAAC |
| | | ACCGAGGAGAAGAUCUACCUGCCCCCCAUCCCCGCCGACGAGAUCCGGAACCCCGUGGUGCUGCGGGCCCUGUCCCAGGCCCGGAAGGUGA |
| | | UCAACGGCGUGGUGCGGCGGUACGGCUCCCCCGCCCGGAUCCACAUCGAGACCGCCCGGGAGGUGGGCAAGUCCUUCAAGGACCGGAAGGA |
| | | GAUCGAGAAGCGGCAGGAGGAGAACCGGAAGGACCGGGAGAAGGCCGCCGCCAAGUUCCGGGAGUACUUCCCCAACUUCGUGGGCGAGCCC |
| | | AAGUCCAAGGACAUCCUGAAGCUGCGGCUGUACGAGCAGCAGCACGGCAAGUGCCUGUACUCCGGCAAGGAGAUCAACCUGGUGCGGCUGA |
| | | ACGAGAAGGGCUACGUGGAGAUCGACCACGCCCUGCCCUUCUCCCGGACCUGGGACGACUCCUUCAACAACAAGGUGCUGGUGCUGGGCUC |
| | | CGAGAACCAGAACAAGGGCAACCAGACCCCCUACGAGUACUUCAACGGCAAGGACAACUCCCGGGAGUGGCAGGAGUUCAAGGCCCGGGUG |
| | | GAGACCUCCCGGUUCCCCCGGUCCAAGAAGCAGCGGAUCCUGCUGCAGAAGUUCGACGAGGACGGCUUCAAGGAGUGCAACCUGAACGACA |
| | | CCCGGUACGUGAACCGCUUCCUGUGCCAGUUCGUGGCCGACCACAUCCUGCUGACCGGCAAGGGCAAGCGGCGGGUGUUCGCCUCCAACGG |
| | | CCAGAUCACCAACCUGCUGCGGGGCUUCUGGGGCCUGCGGAAGGUGCGGGCCGAGAACGACCGGCACCACGCCCUGGACGCCGUGGUGGUG |
| | | GCCUGCUCCACCGUGGCCAUGCAGCAGAAGAUCACCCGGUUCGUGCGGUACAAGGAGAUGAACGCCUUCGACGGCAAGACCAUCGACAAGG |
| | | AGACCGGCAAGGUGCUGCACCAGAAGACCCACUUCCCCCAGCCCUGGGAGUUCUUCGCCCAGGAGGUGAUGAUCCGGGUGUUCGGCAAGCC |
| | | CGACGGCAAGCCCGAGUUCGAGGAGGCCGACACCCCCGAGAAGCUGCGGACCCUGCUGGCCGAGAAGCUGUCCUCCCGGCCCGAGGCCGUG |
| | | CACGAGUACGUGACCCCCCUGUUCGUGUCCCGGGCCCCCAACCGGAAGAUGUCCGGCGCCCACAAGGACACCCUGCGGUCCGCCAAGCGGU |
| | | UCGUGAAGCACAACGAGAAGAUCUCCGUGAAGCGGGUGUGGCUGACCGAGAUCAAGCUGGCCGACCUGGAGAACAUGGUGAACUACAAGAA |
| | | CGGCCGGGAGAUCGAGCUGUACGAGGCCCUGAAGGCCCGGCUGGAGGCCUACGGCGGCAACGCCAAGCAGGCCUUCGACCCCAAGGACAAC |
| | | CCCUUCUACAAGAAGGGCGGCCAGCUGGUGAAGGCCGUGCGGGUGGAGAAGACCCAGGAGUCCGGCGUGCUGCUGAACAAGAAGAACGCCU |
| | | ACACCAUCGCCGACAACGGCGACAUGGUGCGGGUGGACGUGUUCUGCAAGGUGGACAAGAAGGGCAAGAACCAGUACUUCAUCGUGCCCAU |
| | | CUACGCCUGGCAGGUGGCCGAGAACAUCCUGCCCGACAUCGACUGCAAGGGCUACCGGAUCGACGACUCCUACACCUUCUGCUUCUCCCUG |
| | | CACAAGUACGACCUGAUCGCCUUCCAGAAGGACGAGAAGUCCAAGGUGGAGUUCGCCUACUACAUCAACUGCGACUCCUCCAACGGCCGGU |
| | | UCUACCUGGCCUGGCACGACAAGGGCUCCAAGGAGCAGCAGUUCCGGAUCUCCACCCAGAACCUGGUGCUGAUCCAGAAGUACCAGGUGAA |
| | | CGAGCUGGGCAAGGAGAUCCGGCCCUGCCGGCUGAAGAAGCGGCCCCCCGUGCGGUCCGGAAAGCGGACCGCCGACGGCUCCGGAGGAGGA |
| | | AGCCCGAAGAAGAAGAGAAAGGUCAGCGAAAGCGCAACACCGGAAAGCGUCAGCGGAUGGAGACUGUUCAAGAAGAUCAGCUAGCUAGCAC |
| | | CAGCCUCAAGAACACCCGAAUGGAGUCUCUAAGCUACAUAAUACCAACUUACACUUUACAAAAUGUUGUCCCCCAAAAUGUAGCCAUUCGU |
| | | AUCUGCUCCUAAUAAAAAGAAAGUUUCUUCACAUUCUCUCGAGAAAAAAAAAAAAUGGAAAAAAAAAAAACGGAAAAAAAAAAAAGGUAAA |
| | | AAAAAAAAAUAUAAAAAAAAAAAACAUAAAAAAAAAAAACGAAAAAAAAAAAACGUAAAAAAAAAAAACUCAAAAAAAAAAAAGAUAAAAA |
| | | AAAAAAACCUAAAAAAAAAAAAUGUAAAAAAAAAAAAGGGAAAAAAAAAAAACGCAAAAAAAAAAAACACAAAAAAAAAAAAUGCAAAAAA |
| | | AAAAAAUCGAAAAAAAAAAAAUCUAAAAAAAAAAAACGAAAAAAAAAAAACCCAAAAAAAAAAAAGACAAAAAAAAAAAAUAGAAAAAAAA |
| | | AAAAGUUAAAAAAAAAAAACUGAAAAAAAAAAAAUUUAAAAAAAAAAAAUCUAG |
| 64 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 41 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | R)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCGGCUCCUCCUCCGGUGCCGCCUUCAAGCCCAACCCCAUCAACUACAUCCUGGGCCUGGACAU |
| | | CGGCAUCGCCUCCGUGGGCUGGGCCAUGGUGGAGAUCGACGAGGAGGAGAACCCCAUCCGGCUGAUCGACCUGGGCGUGCGGGUGUUCGAG |
| | | CGGGCCGAGGUGCCCAAGACCGGCGACUCCCUGGCCAUGGCCCGGCGGCUGGCCCGGUCCGUGCGGCGGCUGACCCGGCGGCGGGCCCACC |
| | | GGCUGCUGCGGGCCCGGCGGCUGCUGAAGCGGGAGGGCGUGCUGCAGGCCGCCGACUUCGACGAGAACGGCCUGAUCAAGUCCCUGCCCAA |
| | | CACCCCCUGGCAGCUGCGGGCCGCCGCCCUGGACCGGAAGCUGACCCCCCUGGAGUGGUCCGCCGUGCUGCUGCACCUGAUCAAGCACCGG |
| | | GGCUACCUGUCCCAGCGGAAGAACGAGGGCGAGACCGCCGACAAGGAGCUGGGCGCCCUGCUGAAGGGCGUGGCCAACAACGCCCACGCCC |
| | | UGCAGACCGGCGACUUCCGGACCCCCGCCGAGCUGGCCCUGAACAAGUUCGAGAAGGAGUCCGGCCACAUCCGGAACCAGCGGGGCGACUA |
| | | CUCCCACACCUUCUCCCGGAAGGACCUGCAGGCCGAGCUGAUCCUGCUGUUCGAGAAGCAGAAGGAGUUCGGCAACCCCCACGUGUCCGGC |
| | | GGCCUGAAGGAGGGCAUCGAGACCCUGCUGAUGACCCAGCGGCCCGCCCUGUCCGGCGACGCCGUGCAGAAGAUGCUGGGCCACUGCACCU |
| | | UCGAGCCCGCCGAGCCCAAGGCCGCCAAGAACACCUACACCGCCGAGCGGUUCAUCUGGCUGACCAAGCUGAACAACCUGCGGAUCCUGGA |
| | | GCAGGGCUCCGAGCGGCCCCUGACCGACACCGAGCGGGCCACCCUGAUGGACGAGCCCUACCGGAAGUCCAAGCUGACCUACGCCCAGGCC |
| | | CGGAAGCUGCUGGGCCUGGAGGACACCGCCUUCUUCAAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAAGUCCCCCCUGAACCUGUCCUCCGAGCUGCAGGACGA |
| | | GAUCGGCACCGCCUUCUCCCUGUUCAAGACCGACGAGGACAUCACCGGCCGGCUGAAGGACCGGGUGCAGCCCGAGAUCCUGGAGGCCCUG |
| | | CUGAAGCACAUCUCCUUCGACAAGUUCGUGCAGAUCUCCCUGAAGGCCCUGCGGCGGAUCGUGCCCCUGAUGGAGCAGGGCAAGCGGUACG |
| | | ACGAGGCCUGCGCCGAGAUCUACGGCGACCACUACGGCAAGAAGAACACCGAGGAGAAGAUCUACCUGCCCCCCAUCCCCGCCGACGAGAU |
| | | CCGGAACCCCGUGGUGCUGCGGGCCCUGUCCCAGGCCCGGAAGGUGAUCAACGGCGUGGUGCGGCGGUACGGCUCCCCCGCCCGGAUCCAC |
| | | AUCGAGACCGCCCGGGAGGUGGGCAAGUCCUUCAAGGACCGGAAGGAGAUCGAGAAGCGGCAGGAGGAGAACCGGAAGGACCGGGAGAAGG |
| | | CCGCCGCCAAGUUCCGGGAGUACUUCCCCAACUUCGUGGGCGAGCCCAAGUCCAAGGACAUCCUGAAGCUGCGGCUGUACGAGCAGCAGCA |
| | | CGGCAAGUGCCUGUACUCCGGCAAGGAGAUCAACCUGGUGCGGCUGAACGAGAAGGGCUACGUGGAGAUCGACCACGCCCUGCCCUUCUCC |
| | | CGGACCUGGGACGACUCCUUCAACAACAAGGUGCUGGUGCUGGGCUCCGAGAACCAGAACAAGGGCAACCAGACCCCCUACGAGUACUUCA |
| | | ACGGCAAGGACAACUCCCGGGAGUGGCAGGAGUUCAAGGCCCGGGUGGAGACCUCCCGGUUCCCCCGGUCCAAGAAGCAGCGGAUCCUGCU |
| | | GCAGAAGUUCGACGAGGACGGCUUCAAGGAGUGCAACCUGAACGACACCCGGUACGUGAACCGCUUCCUGUGCCAGUUCGUGGCCGACCAC |
| | | AUCCUGCUGACCGGCAAGGGCAAGCGGCGGGUGUUCGCCUCCAACGGCCAGAUCACCAACCUGCUGCGGGGCUUCUGGGGCCUGCGGAAGG |
| | | UGCGGGCCGAGAACGACCGGCACCACGCCCUGGACGCCGUGGUGGUGGCCUGCUCCACCGUGGCCAUGCAGCAGAAGAUCACCCGGUUCGU |
| | | GCGGUACAAGGAGAUGAACGCCUUCGACGGCAAGACCAUCGACAAGGAGACCGGCAAGGUGCUGCACCAGAAGACCCACUUCCCCCAGCCC |
| | | UGGGAGUUCUUCGCCCAGGAGGUGAUGAUCCGGGUGUUCGGCAAGCCCGACGGCAAGCCCGAGUUCGAGGAGGCCGACACCCCCGAGAAGC |
| | | UGCGGACCCUGCUGGCCGAGAAGCUGUCCUCCCGGCCCGAGGCCGUGCACGAGUACGUGACCCCCCUGUUCGUGUCCCGGGCCCCCAACCG |
| | | GAAGAUGUCCGGCGCCCACAAGGACACCCUGCGGUCCGCCAAGCGGUUCGUGAAGCACAACGAGAAGAUCUCCGUGAAGCGGGUGUGGCUG |
| | | ACCGAGAUCAAGCUGGCCGACCUGGAGAACAUGGUGAACUACAAGAACGGCCGGGAGAUCGAGCUGUACGAGGCCCUGAAGGCCCGGCUGG |
| | | AGGCCUACGGCGGCAACGCCAAGCAGGCCUUCGACCCCAAGGACAACCCCUUCUACAAGAAGGGCGGCCAGCUGGUGAAGGCCGUGCGGGU |
| | | GGAGAAGACCCAGGAGUCCGGCGUGCUGCUGAACAAGAAGAACGCCUACACCAUCGCCGACAACGGCGACAUGGUGCGGGUGGACGUGUUC |
| | | UGCAAGGUGGACAAGAAGGGCAAGAACCAGUACUUCAUCGUGCCCAUCUACGCCUGGCAGGUGGCCGAGAACAUCCUGCCCGACAUCGACU |
| | | GCAAGGGCUACCGGAUCGACGACUCCUACACCUUCUGCUUCUCCCUGCACAAGUACGACCUGAUCGCCUUCCAGAAGGACGAGAAGUCCAA |
| | | GGUGGAGUUCGCCUACUACAUCAACUGCGACUCCUCCAACGGCCGGUUCUACCUGGCCUGGCACGACAAGGGCUCCAAGGAGCAGCAGUUC |
| | | CGGAUCUCCACCCAGAACCUGGUGCUGAUCCAGAAGUACCAGGUGAACGAGCUGGGCAAGGAGAUCCGGCCCUGCCGGCUGAAGAAGCGGC |
| | | CCCCCGUGCGGUCCGGAAAGCGGACCGCCGACGGCUCCGGAGGAGGAAGCCCGAAGAAGAAGAGAAAGGUCAGCGAAAGCGCAACACCGGA |
| | | AAGCGUCAGCGGAUGGAGACUGUUCAAGAAGAUCAGCUAG |
| 65 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 41 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | R)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATESGGTSTESEGSAGTSTESEGSAGSAGSTSGSSSGAAFKPNPINYILGLDIGIASVGWAMVEIDEEENPIRLIDLGVRVFE |
| | | RAEVPKTGDSLAMARRLARSVRRLTRRRAHRLLRARRLLKREGVLQAADFDENGLIKSLPNTPWQLRAAALDRKLTPLEWSAVLLHLIKHR |
| | | GYLSQRKNEGETADKELGALLKGVANNAHALQTGDFRTPAELALNKFEKESGHIRNQRGDYSHTFSRKDLQAELILLFEKQKEFGNPHVSG |
| | | GLKEGIETLLMTQRPALSGDAVQKMLGHCTFEPAEPKAAKNTYTAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKDKKSPLNLSSELQDEIGTAFSLFKTDEDITGRLKDRVQPEILEAL |
| | | LKHISFDKFVQISLKALRRIVPLMEQGKRYDEACAEIYGDHYGKKNTEEKIYLPPIPADEIRNPVVLRALSQARKVINGVVRRYGSPARIH |
| | | IETAREVGKSFKDRKEIEKRQEENRKDREKAAAKFREYFPNFVGEPKSKDILKLRLYEQQHGKCLYSGKEINLVRLNEKGYVEIDHALPFS |
| | | RTWDDSFNNKVLVLGSENQNKGNQTPYEYFNGKDNSREWQEFKARVETSRFPRSKKQRILLQKFDEDGFKECNLNDTRYVNRFLCQFVADH |
| | | ILLTGKGKRRVFASNGQITNLLRGFWGLRKVRAENDRHHALDAVVVACSTVAMQQKITRFVRYKEMNAFDGKTIDKETGKVLHQKTHFPQP |
| | | WEFFAQEVMIRVFGKPDGKPEFEEADTPEKLRTLLAEKLSSRPEAVHEYVTPLFVSRAPNRKMSGAHKDTLRSAKRFVKHNEKISVKRVWL |
| | | TEIKLADLENMVNYKNGREIELYEALKARLEAYGGNAKQAFDPKDNPFYKKGGQLVKAVRVEKTQESGVLLNKKNAYTIADNGDMVRVDVF |
| | | CKVDKKGKNQYFIVPIYAWQVAENILPDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVRSGKRTADGSGGGSPKKKRKVSESATPESVSGWRLFKKIS* |
| 66 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 41 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | R)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCGCCGGCUCCGCCGGCUCCACCUCCGGCUCCUCCUCCGGUGCCGCCUUCAAGCCCAACCCCAUCAACUACAUCCUGGGCCUGGACAU |
| | | CGGCAUCGCCUCCGUGGGCUGGGCCAUGGUGGAGAUCGACGAGGAGGAGAACCCCAUCCGGCUGAUCGACCUGGGCGUGCGGGUGUUCGAG |
| | | CGGGCCGAGGUGCCCAAGACCGGCGACUCCCUGGCCAUGGCCCGGCGGCUGGCCCGGUCCGUGCGGCGGCUGACCCGGCGGCGGGCCCACC |
| | | GGCUGCUGCGGGCCCGGCGGCUGCUGAAGCGGGAGGGCGUGCUGCAGGCCGCCGACUUCGACGAGAACGGCCUGAUCAAGUCCCUGCCCAA |
| | | CACCCCCUGGCAGCUGCGGGCCGCCGCCCUGGACCGGAAGCUGACCCCCCUGGAGUGGUCCGCCGUGCUGCUGCACCUGAUCAAGCACCGG |
| | | GGCUACCUGUCCCAGCGGAAGAACGAGGGCGAGACCGCCGACAAGGAGCUGGGCGCCCUGCUGAAGGGCGUGGCCAACAACGCCCACGCCC |
| | | UGCAGACCGGCGACUUCCGGACCCCCGCCGAGCUGGCCCUGAACAAGUUCGAGAAGGAGUCCGGCCACAUCCGGAACCAGCGGGGCGACUA |
| | | CUCCCACACCUUCUCCCGGAAGGACCUGCAGGCCGAGCUGAUCCUGCUGUUCGAGAAGCAGAAGGAGUUCGGCAACCCCCACGUGUCCGGC |
| | | GGCCUGAAGGAGGGCAUCGAGACCCUGCUGAUGACCCAGCGGCCCGCCCUGUCCGGCGACGCCGUGCAGAAGAUGCUGGGCCACUGCACCU |
| | | UCGAGCCCGCCGAGCCCAAGGCCGCCAAGAACACCUACACCGCCGAGCGGUUCAUCUGGCUGACCAAGCUGAACAACCUGCGGAUCCUGGA |
| | | GCAGGGCUCCGAGCGGCCCCUGACCGACACCGAGCGGGCCACCCUGAUGGACGAGCCCUACCGGAAGUCCAAGCUGACCUACGCCCAGGCC |
| | | CGGAAGCUGCUGGGCCUGGAGGACACCGCCUUCUUCAAGGGCCUGCGGUACGGCAAGGACAACGCCGAGGCCUCCACCCUGAUGGAGAUGA |
| | | AGGCCUACCACGCCAUCUCCCGGGCCCUGGAGAAGGAGGGCCUGAAGGACAAGAAGUCCCCCCUGAACCUGUCCUCCGAGCUGCAGGACGA |
| | | GAUCGGCACCGCCUUCUCCCUGUUCAAGACCGACGAGGACAUCACCGGCCGGCUGAAGGACCGGGUGCAGCCCGAGAUCCUGGAGGCCCUG |
| | | CUGAAGCACAUCUCCUUCGACAAGUUCGUGCAGAUCUCCCUGAAGGCCCUGCGGCGGAUCGUGCCCCUGAUGGAGCAGGGCAAGCGGUACG |
| | | ACGAGGCCUGCGCCGAGAUCUACGGCGACCACUACGGCAAGAAGAACACCGAGGAGAAGAUCUACCUGCCCCCCAUCCCCGCCGACGAGAU |
| | | CCGGAACCCCGUGGUGCUGCGGGCCCUGUCCCAGGCCCGGAAGGUGAUCAACGGCGUGGUGCGGCGGUACGGCUCCCCCGCCCGGAUCCAC |
| | | AUCGAGACCGCCCGGGAGGUGGGCAAGUCCUUCAAGGACCGGAAGGAGAUCGAGAAGCGGCAGGAGGAGAACCGGAAGGACCGGGAGAAGG |
| | | CCGCCGCCAAGUUCCGGGAGUACUUCCCCAACUUCGUGGGCGAGCCCAAGUCCAAGGACAUCCUGAAGCUGCGGCUGUACGAGCAGCAGCA |
| | | CGGCAAGUGCCUGUACUCCGGCAAGGAGAUCAACCUGGUGCGGCUGAACGAGAAGGGCUACGUGGAGAUCGACCACGCCCUGCCCUUCUCC |
| | | CGGACCUGGGACGACUCCUUCAACAACAAGGUGCUGGUGCUGGGCUCCGAGAACCAGAACAAGGGCAACCAGACCCCCUACGAGUACUUCA |
| | | ACGGCAAGGACAACUCCCGGGAGUGGCAGGAGUUCAAGGCCCGGGUGGAGACCUCCCGGUUCCCCCGGUCCAAGAAGCAGCGGAUCCUGCU |
| | | GCAGAAGUUCGACGAGGACGGCUUCAAGGAGUGCAACCUGAACGACACCCGGUACGUGAACCGCUUCCUGUGCCAGUUCGUGGCCGACCAC |
| | | AUCCUGCUGACCGGCAAGGGCAAGCGGCGGGUGUUCGCCUCCAACGGCCAGAUCACCAACCUGCUGCGGGGCUUCUGGGGCCUGCGGAAGG |
| | | UGCGGGCCGAGAACGACCGGCACCACGCCCUGGACGCCGUGGUGGUGGCCUGCUCCACCGUGGCCAUGCAGCAGAAGAUCACCCGGUUCGU |
| | | GCGGUACAAGGAGAUGAACGCCUUCGACGGCAAGACCAUCGACAAGGAGACCGGCAAGGUGCUGCACCAGAAGACCCACUUCCCCCAGCCC |
| | | UGGGAGUUCUUCGCCCAGGAGGUGAUGAUCCGGGUGUUCGGCAAGCCCGACGGCAAGCCCGAGUUCGAGGAGGCCGACACCCCCGAGAAGC |
| | | UGCGGACCCUGCUGGCCGAGAAGCUGUCCUCCCGGCCCGAGGCCGUGCACGAGUACGUGACCCCCCUGUUCGUGUCCCGGGCCCCCAACCG |
| | | GAAGAUGUCCGGCGCCCACAAGGACACCCUGCGGUCCGCCAAGCGGUUCGUGAAGCACAACGAGAAGAUCUCCGUGAAGCGGGUGUGGCUG |
| | | ACCGAGAUCAAGCUGGCCGACCUGGAGAACAUGGUGAACUACAAGAACGGCCGGGAGAUCGAGCUGUACGAGGCCCUGAAGGCCCGGCUGG |
| | | AGGCCUACGGCGGCAACGCCAAGCAGGCCUUCGACCCCAAGGACAACCCCUUCUACAAGAAGGGCGGCCAGCUGGUGAAGGCCGUGCGGGU |
| | | GGAGAAGACCCAGGAGUCCGGCGUGCUGCUGAACAAGAAGAACGCCUACACCAUCGCCGACAACGGCGACAUGGUGCGGGUGGACGUGUUC |
| | | UGCAAGGUGGACAAGAAGGGCAAGAACCAGUACUUCAUCGUGCCCAUCUACGCCUGGCAGGUGGCCGAGAACAUCCUGCCCGACAUCGACU |
| | | GCAAGGGCUACCGGAUCGACGACUCCUACACCUUCUGCUUCUCCCUGCACAAGUACGACCUGAUCGCCUUCCAGAAGGACGAGAAGUCCAA |
| | | GGUGGAGUUCGCCUACUACAUCAACUGCGACUCCUCCAACGGCCGGUUCUACCUGGCCUGGCACGACAAGGGCUCCAAGGAGCAGCAGUUC |
| | | CGGAUCUCCACCCAGAACCUGGUGCUGAUCCAGAAGUACCAGGUGAACGAGCUGGGCAAGGAGAUCCGGCCCUGCCGGCUGAAGAAGCGGC |
| | | CCCCCGUGCGGUCCGGAAAGCGGACCGCCGACGGCUCCGGAGGAGGAAGCCCGAAGAAGAAGAGAAAGGUCUAG |
| 67 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 41 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | R)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | mRNA | LDKAGFIKRQLVETRQITKHVAQILDSRMNTKYDENDKLIREVKVITLKSKLVSDFRKDFQFYKVREINNYHHAHDAYLNAVVGTALIKKY |
| | sequence | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSGSSSGAAFKPNPINYILGLDIGIASVGWAMVEIDEEENPIRLIDLGVRVFE |
| | | RAEVPKTGDSLAMARRLARSVRRLTRRRAHRLLRARRLLKREGVLQAADFDENGLIKSLPNTPWQLRAAALDRKLTPLEWSAVLLHLIKHR |
| | | GYLSQRKNEGETADKELGALLKGVANNAHALQTGDFRTPAELALNKFEKESGHIRNQRGDYSHTFSRKDLQAELILLFEKQKEFGNPHVSG |
| | | GLKEGIETLLMTQRPALSGDAVQKMLGHCTFEPAEPKAAKNTYTAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKDKKSPLNLSSELQDEIGTAFSLFKTDEDITGRLKDRVQPEILEAL |
| | | LKHISFDKFVQISLKALRRIVPLMEQGKRYDEACAEIYGDHYGKKNTEEKIYLPPIPADEIRNPVVLRALSQARKVINGVVRRYGSPARIH |
| | | IETAREVGKSFKDRKEIEKRQEENRKDREKAAAKFREYFPNFVGEPKSKDILKLRLYEQQHGKCLYSGKEINLVRLNEKGYVEIDHALPFS |
| | | RTWDDSFNNKVLVLGSENQNKGNQTPYEYFNGKDNSREWQEFKARVETSRFPRSKKQRILLQKFDEDGFKECNLNDTRYVNRFLCQFVADH |
| | | ILLTGKGKRRVFASNGQITNLLRGFWGLRKVRAENDRHHALDAVVVACSTVAMQQKITRFVRYKEMNAFDGKTIDKETGKVLHQKTHFPQP |
| | | WEFFAQEVMIRVFGKPDGKPEFEEADTPEKLRTLLAEKLSSRPEAVHEYVTPLFVSRAPNRKMSGAHKDTLRSAKRFVKHNEKISVKRVWL |
| | | TEIKLADLENMVNYKNGREIELYEALKARLEAYGGNAKQAFDPKDNPFYKKGGQLVKAVRVEKTQESGVLLNKKNAYTIADNGDMVRVDVF |
| | | CKVDKKGKNQYFIVPIYAWQVAENILPDIDCKGYRIDDSYTFCFSLHKYDLIAFQKDEKSKVEFAYYINCDSSNGRFYLAWHDKGSKEQQF |
| | | RISTQNLVLIQKYQVNELGKEIRPCRLKKRPPVRSGKRTADGSGGGSPKKKRKV* |
| 68 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 31 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | S)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGAGUCCGGCGGCACCUC |
| | | CACCGAGUCCGAGGGCUCCGCCGGCACCUCCACCGAGUCCGAGGGCUCCUCCUCCGGUGCCGCCUUCAAGCCCAACCCCAUCAACUACAUC |
| | | CUGGGCCUGGACAUCGGCAUCGCCUCCGUGGGCUGGGCCAUGGUGGAGAUCGACGAGGAGGAGAACCCCAUCCGGCUGAUCGACCUGGGCG |
| | | UGCGGGUGUUCGAGCGGGCCGAGGUGCCCAAGACCGGCGACUCCCUGGCCAUGGCCCGGCGGCUGGCCCGGUCCGUGCGGCGGCUGACCCG |
| | | GCGGCGGGCCCACCGGCUGCUGCGGGCCCGGCGGCUGCUGAAGCGGGAGGGCGUGCUGCAGGCCGCCGACUUCGACGAGAACGGCCUGAUC |
| | | AAGUCCCUGCCCAACACCCCCUGGCAGCUGCGGGCCGCCGCCCUGGACCGGAAGCUGACCCCCCUGGAGUGGUCCGCCGUGCUGCUGCACC |
| | | UGAUCAAGCACCGGGGCUACCUGUCCCAGCGGAAGAACGAGGGCGAGACCGCCGACAAGGAGCUGGGCGCCCUGCUGAAGGGCGUGGCCAA |
| | | CAACGCCCACGCCCUGCAGACCGGCGACUUCCGGACCCCCGCCGAGCUGGCCCUGAACAAGUUCGAGAAGGAGUCCGGCCACAUCCGGAAC |
| | | CAGCGGGGCGACUACUCCCACACCUUCUCCCGGAAGGACCUGCAGGCCGAGCUGAUCCUGCUGUUCGAGAAGCAGAAGGAGUUCGGCAACC |
| | | CCCACGUGUCCGGCGGCCUGAAGGAGGGCAUCGAGACCCUGCUGAUGACCCAGCGGCCCGCCCUGUCCGGCGACGCCGUGCAGAAGAUGCU |
| | | GGGCCACUGCACCUUCGAGCCCGCCGAGCCCAAGGCCGCCAAGAACACCUACACCGCCGAGCGGUUCAUCUGGCUGACCAAGCUGAACAAC |
| | | CUGCGGAUCCUGGAGCAGGGCUCCGAGCGGCCCCUGACCGACACCGAGCGGGCCACCCUGAUGGACGAGCCCUACCGGAAGUCCAAGCUGA |
| | | CCUACGCCCAGGCCCGGAAGCUGCUGGGCCUGGAGGACACCGCCUUCUUCAAGGGCCUGCGGUACGGCAAGGACAACGCCGAGGCCUCCAC |
| | | CCUGAUGGAGAUGAAGGCCUACCACGCCAUCUCCCGGGCCCUGGAGAAGGAGGGCCUGAAGGACAAGAAGUCCCCCCUGAACCUGUCCUCC |
| | | GAGCUGCAGGACGAGAUCGGCACCGCCUUCUCCCUGUUCAAGACCGACGAGGACAUCACCGGCCGGCUGAAGGACCGGGUGCAGCCCGAGA |
| | | UCCUGGAGGCCCUGCUGAAGCACAUCUCCUUCGACAAGUUCGUGCAGAUCUCCCUGAAGGCCCUGCGGCGGAUCGUGCCCCUGAUGGAGCA |
| | | GGGCAAGCGGUACGACGAGGCCUGCGCCGAGAUCUACGGCGACCACUACGGCAAGAAGAACACCGAGGAGAAGAUCUACCUGCCCCCCAUC |
| | | CCCGCCGACGAGAUCCGGAACCCCGUGGUGCUGCGGGCCCUGUCCCAGGCCCGGAAGGUGAUCAACGGCGUGGUGCGGCGGUACGGCUCCC |
| | | CCGCCCGGAUCCACAUCGAGACCGCCCGGGAGGUGGGCAAGUCCUUCAAGGACCGGAAGGAGAUCGAGAAGCGGCAGGAGGAGAACCGGAA |
| | | GGACCGGGAGAAGGCCGCCGCCAAGUUCCGGGAGUACUUCCCCAACUUCGUGGGCGAGCCCAAGUCCAAGGACAUCCUGAAGCUGCGGCUG |
| | | UACGAGCAGCAGCACGGCAAGUGCCUGUACUCCGGCAAGGAGAUCAACCUGGUGCGGCUGAACGAGAAGGGCUACGUGGAGAUCGACCACG |
| | | CCCUGCCCUUCUCCCGGACCUGGGACGACUCCUUCAACAACAAGGUGCUGGUGCUGGGCUCCGAGAACCAGAACAAGGGCAACCAGACCCC |
| | | CUACGAGUACUUCAACGGCAAGGACAACUCCCGGGAGUGGCAGGAGUUCAAGGCCCGGGUGGAGACCUCCCGGUUCCCCCGGUCCAAGAAG |
| | | CAGCGGAUCCUGCUGCAGAAGUUCGACGAGGACGGCUUCAAGGAGUGCAACCUGAACGACACCCGGUACGUGAACCGCUUCCUGUGCCAGU |
| | | UCGUGGCCGACCACAUCCUGCUGACCGGCAAGGGCAAGCGGCGGGUGUUCGCCUCCAACGGCCAGAUCACCAACCUGCUGCGGGGCUUCUG |
| | | GGGCCUGCGGAAGGUGCGGGCCGAGAACGACCGGCACCACGCCCUGGACGCCGUGGUGGUGGCCUGCUCCACCGUGGCCAUGCAGCAGAAG |
| | | AUCACCCGGUUCGUGCGGUACAAGGAGAUGAACGCCUUCGACGGCAAGACCAUCGACAAGGAGACCGGCAAGGUGCUGCACCAGAAGACCC |
| | | ACUUCCCCCAGCCCUGGGAGUUCUUCGCCCAGGAGGUGAUGAUCCGGGUGUUCGGCAAGCCCGACGGCAAGCCCGAGUUCGAGGAGGCCGA |
| | | CACCCCCGAGAAGCUGCGGACCCUGCUGGCCGAGAAGCUGUCCUCCCGGCCCGAGGCCGUGCACGAGUACGUGACCCCCCUGUUCGUGUCC |
| | | CGGGCCCCCAACCGGAAGAUGUCCGGCGCCCACAAGGACACCCUGCGGUCCGCCAAGCGGUUCGUGAAGCACAACGAGAAGAUCUCCGUGA |
| | | AGCGGGUGUGGCUGACCGAGAUCAAGCUGGCCGACCUGGAGAACAUGGUGAACUACAAGAACGGCCGGGAGAUCGAGCUGUACGAGGCCCU |
| | | GAAGGCCCGGCUGGAGGCCUACGGCGGCAACGCCAAGCAGGCCUUCGACCCCAAGGACAACCCCUUCUACAAGAAGGGCGGCCAGCUGGUG |
| | | AAGGCCGUGCGGGUGGAGAAGACCCAGGAGUCCGGCGUGCUGCUGAACAAGAAGAACGCCUACACCAUCGCCGACAACGGCGACAUGGUGC |
| | | GGGUGGACGUGUUCUGCAAGGUGGACAAGAAGGGCAAGAACCAGUACUUCAUCGUGCCCAUCUACGCCUGGCAGGUGGCCGAGAACAUCCU |
| | | GCCCGACAUCGACUGCAAGGGCUACCGGAUCGACGACUCCUACACCUUCUGCUUCUCCCUGCACAAGUACGACCUGAUCGCCUUCCAGAAG |
| | | GACGAGAAGUCCAAGGUGGAGUUCGCCUACUACAUCAACUGCGACUCCUCCAACGGCCGGUUCUACCUGGCCUGGCACGACAAGGGCUCCA |
| | | AGGAGCAGCAGUUCCGGAUCUCCACCCAGAACCUGGUGCUGAUCCAGAAGUACCAGGUGAACGAGCUGGGCAAGGAGAUCCGGCCCUGCCG |
| | | GCUGAAGAAGCGGCCCCCCGUGCGGUCCGGAAAGCGGACCGCCGACGGCUCCGGAGGAGGAAGCCCGAAGAAGAAGAGAAAGGUCAGCGAA |
| | | AGCGCAACACCGGAAAGCGUCAGCGGAUGGAGACUGUUCAAGAAGAUCAGCUAGCUAGCACCAGCCUCAAGAACACCCGAAUGGAGUCUCU |
| | | AAGCUACAUAAUACCAACUUACACUUUACAAAAUGUUGUCCCCCAAAAUGUAGCCAUUCGUAUCUGCUCCUAAUAAAAAGAAAGUUUCUUC |
| | | ACAUUCUCUCGAGAAAAAAAAAAAAUGGAAAAAAAAAAAACGGAAAAAAAAAAAAGGUAAAAAAAAAAAAUAUAAAAAAAAAAAACAUAAA |
| | | AAAAAAAAACGAAAAAAAAAAAACGUAAAAAAAAAAAACUCAAAAAAAAAAAAGAUAAAAAAAAAAAACCUAAAAAAAAAAAAUGUAAAAA |
| | | AAAAAAAGGGAAAAAAAAAAAACGCAAAAAAAAAAAACACAAAAAAAAAAAAUGCAAAAAAAAAAAAUCGAAAAAAAAAAAAUCUAAAAAA |
| | | AAAAAACGAAAAAAAAAAAACCCAAAAAAAAAAAAGACAAAAAAAAAAAAUAGAAAAAAAAAAAAGUUAAAAAAAAAAAACUGAAAAAAAA |
| | | AAAAUUUAAAAAAAAAAAAUCUAG |
| 69 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 31 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | S)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCUCCUCCGGUGCCGCCUUCAAGCCCAACCCCAUCAACUACAUCCUGGGCCUGGACAUCGGCAUCGCCUCCGUGGGCUGGGCCAUGGU |
| | | GGAGAUCGACGAGGAGGAGAACCCCAUCCGGCUGAUCGACCUGGGCGUGCGGGUGUUCGAGCGGGCCGAGGUGCCCAAGACCGGCGACUCC |
| | | CUGGCCAUGGCCCGGCGGCUGGCCCGGUCCGUGCGGCGGCUGACCCGGCGGCGGGCCCACCGGCUGCUGCGGGCCCGGCGGCUGCUGAAGC |
| | | GGGAGGGCGUGCUGCAGGCCGCCGACUUCGACGAGAACGGCCUGAUCAAGUCCCUGCCCAACACCCCCUGGCAGCUGCGGGCCGCCGCCCU |
| | | GGACCGGAAGCUGACCCCCCUGGAGUGGUCCGCCGUGCUGCUGCACCUGAUCAAGCACCGGGGCUACCUGUCCCAGCGGAAGAACGAGGGC |
| | | GAGACCGCCGACAAGGAGCUGGGCGCCCUGCUGAAGGGCGUGGCCAACAACGCCCACGCCCUGCAGACCGGCGACUUCCGGACCCCCGCCG |
| | | AGCUGGCCCUGAACAAGUUCGAGAAGGAGUCCGGCCACAUCCGGAACCAGCGGGGCGACUACUCCCACACCUUCUCCCGGAAGGACCUGCA |
| | | GGCCGAGCUGAUCCUGCUGUUCGAGAAGCAGAAGGAGUUCGGCAACCCCCACGUGUCCGGCGGCCUGAAGGAGGGCAUCGAGACCCUGCUG |
| | | AUGACCCAGCGGCCCGCCCUGUCCGGCGACGCCGUGCAGAAGAUGCUGGGCCACUGCACCUUCGAGCCCGCCGAGCCCAAGGCCGCCAAGA |
| | | ACACCUACACCGCCGAGCGGUUCAUCUGGCUGACCAAGCUGAACAACCUGCGGAUCCUGGAGCAGGGCUCCGAGCGGCCCCUGACCGACAC |
| | | CGAGCGGGCCACCCUGAUGGACGAGCCCUACCGGAAGUCCAAGCUGACCUACGCCCAGGCCCGGAAGCUGCUGGGCCUGGAGGACACCGCC |
| | | UUCUUCAAGGGCCUGCGGUACGGCAAGGACAACGCCGAGGCCUCCACCCUGAUGGAGAUGAAGGCCUACCACGCCAUCUCCCGGGCCCUGG |
| | | AGAAGGAGGGCCUGAAGGACAAGAAGUCCCCCCUGAACCUGUCCUCCGAGCUGCAGGACGAGAUCGGCACCGCCUUCUCCCUGUUCAAGAC |
| | | CGACGAGGACAUCACCGGCCGGCUGAAGGACCGGGUGCAGCCCGAGAUCCUGGAGGCCCUGCUGAAGCACAUCUCCUUCGACAAGUUCGUG |
| | | CAGAUCUCCCUGAAGGCCCUGCGGCGGAUCGUGCCCCUGAUGGAGCAGGGCAAGCGGUACGACGAGGCCUGCGCCGAGAUCUACGGCGACC |
| | | ACUACGGCAAGAAGAACACCGAGGAGAAGAUCUACCUGCCCCCCAUCCCCGCCGACGAGAUCCGGAACCCCGUGGUGCUGCGGGCCCUGUC |
| | | CCAGGCCCGGAAGGUGAUCAACGGCGUGGUGCGGCGGUACGGCUCCCCCGCCCGGAUCCACAUCGAGACCGCCCGGGAGGUGGGCAAGUCC |
| | | UUCAAGGACCGGAAGGAGAUCGAGAAGCGGCAGGAGGAGAACCGGAAGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGCUGUACGAGCAGCAGCACGGCAAGUGCCUGUACUCCGGCAAGGAGAU |
| | | CAACCUGGUGCGGCUGAACGAGAAGGGCUACGUGGAGAUCGACCACGCCCUGCCCUUCUCCCGGACCUGGGACGACUCCUUCAACAACAAG |
| | | GUGCUGGUGCUGGGCUCCGAGAACCAGAACAAGGGCAACCAGACCCCCUACGAGUACUUCAACGGCAAGGACAACUCCCGGGAGUGGCAGG |
| | | AGUUCAAGGCCCGGGUGGAGACCUCCCGGUUCCCCCGGUCCAAGAAGCAGCGGAUCCUGCUGCAGAAGUUCGACGAGGACGGCUUCAAGGA |
| | | GUGCAACCUGAACGACACCCGGUACGUGAACCGCUUCCUGUGCCAGUUCGUGGCCGACCACAUCCUGCUGACCGGCAAGGGCAAGCGGCGG |
| | | GUGUUCGCCUCCAACGGCCAGAUCACCAACCUGCUGCGGGGCUUCUGGGGCCUGCGGAAGGUGCGGGCCGAGAACGACCGGCACCACGCCC |
| | | UGGACGCCGUGGUGGUGGCCUGCUCCACCGUGGCCAUGCAGCAGAAGAUCACCCGGUUCGUGCGGUACAAGGAGAUGAACGCCUUCGACGG |
| | | CAAGACCAUCGACAAGGAGACCGGCAAGGUGCUGCACCAGAAGACCCACUUCCCCCAGCCCUGGGAGUUCUUCGCCCAGGAGGUGAUGAUC |
| | | CGGGUGUUCGGCAAGCCCGACGGCAAGCCCGAGUUCGAGGAGGCCGACACCCCCGAGAAGCUGCGGACCCUGCUGGCCGAGAAGCUGUCCU |
| | | CCCGGCCCGAGGCCGUGCACGAGUACGUGACCCCCCUGUUCGUGUCCCGGGCCCCCAACCGGAAGAUGUCCGGCGCCCACAAGGACACCCU |
| | | GCGGUCCGCCAAGCGGUUCGUGAAGCACAACGAGAAGAUCUCCGUGAAGCGGGUGUGGCUGACCGAGAUCAAGCUGGCCGACCUGGAGAAC |
| | | AUGGUGAACUACAAGAACGGCCGGGAGAUCGAGCUGUACGAGGCCCUGAAGGCCCGGCUGGAGGCCUACGGCGGCAACGCCAAGCAGGCCU |
| | | UCGACCCCAAGGACAACCCCUUCUACAAGAAGGGCGGCCAGCUGGUGAAGGCCGUGCGGGUGGAGAAGACCCAGGAGUCCGGCGUGCUGCU |
| | | GAACAAGAAGAACGCCUACACCAUCGCCGACAACGGCGACAUGGUGCGGGUGGACGUGUUCUGCAAGGUGGACAAGAAGGGCAAGAACCAG |
| | | UACUUCAUCGUGCCCAUCUACGCCUGGCAGGUGGCCGAGAACAUCCUGCCCGACAUCGACUGCAAGGGCUACCGGAUCGACGACUCCUACA |
| | | CCUUCUGCUUCUCCCUGCACAAGUACGACCUGAUCGCCUUCCAGAAGGACGAGAAGUCCAAGGUGGAGUUCGCCUACUACAUCAACUGCGA |
| | | CUCCUCCAACGGCCGGUUCUACCUGGCCUGGCACGACAAGGGCUCCAAGGAGCAGCAGUUCCGGAUCUCCACCCAGAACCUGGUGCUGAUC |
| | | CAGAAGUACCAGGUGAACGAGCUGGGCAAGGAGAUCCGGCCCUGCCGGCUGAAGAAGCGGCCCCCCGUGCGGUCCGGAAAGCGGACCGCCG |
| | | ACGGCUCCGGAGGAGGAAGCCCGAAGAAGAAGAGAAAGGUCAGCGAAAGCGCAACACCGGAAAGCGUCAGCGGAUGGAGACUGUUCAAGAA |
| | | GAUCAGCUAG |
| 70 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 31 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | S)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATESGGTSTESEGSAGTSTESEGSSSGAAFKPNPINYILGLDIGIASVGWAMVEIDEEENPIRLIDLGVRVFERAEVPKTGDS |
| | | LAMARRLARSVRRLTRRRAHRLLRARRLLKREGVLQAADFDENGLIKSLPNTPWQLRAAALDRKLTPLEWSAVLLHLIKHRGYLSQRKNEG |
| | | ETADKELGALLKGVANNAHALQTGDFRTPAELALNKFEKESGHIRNQRGDYSHTFSRKDLQAELILLFEKQKEFGNPHVSGGLKEGIETLL |
| | | MTQRPALSGDAVQKMLGHCTFEPAEPKAAKNTYTAERFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSSELQDEIGTAFSLFKTDEDITGRLKDRVQPEILEALLKHISFDKFV |
| | | QISLKALRRIVPLMEQGKRYDEACAEIYGDHYGKKNTEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCLYSGKEINLVRLNEKGYVEIDHALPFSRTWDDSFNNK |
| | | VLVLGSENQNKGNQTPYEYFNGKDNSREWQEFKARVETSRFPRSKKQRILLQKFDEDGFKECNLNDTRYVNRFLCQFVADHILLTGKGKRR |
| | | VFASNGQITNLLRGFWGLRKVRAENDRHHALDAVVVACSTVAMQQKITRFVRYKEMNAFDGKTIDKETGKVLHQKTHFPQPWEFFAQEVMI |
| | | RVFGKPDGKPEFEEADTPEKLRTLLAEKLSSRPEAVHEYVTPLFVSRAPNRKMSGAHKDTLRSAKRFVKHNEKISVKRVWLTEIKLADLEN |
| | | MVNYKNGREIELYEALKARLEAYGGNAKQAFDPKDNPFYKKGGQLVKAVRVEKTQESGVLLNKKNAYTIADNGDMVRVDVFCKVDKKGKNQ |
| | | YFIVPIYAWQVAENILPDIDCKGYRIDDSYTFCFSLHKYDLIAFQKDEKSKVEFAYYINCDSSNGRFYLAWHDKGSKEQQFRISTQNLVLI |
| | | QKYQVNELGKEIRPCRLKKRPPVRSGKRTADGSGGGSPKKKRKVSESATPESVSGWRLFKKIS* |
| 71 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 31 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | S)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCGCCGGCACCUCCACCGAGUCCGAGG |
| | | GCUCCUCCUCCGGUGCCGCCUUCAAGCCCAACCCCAUCAACUACAUCCUGGGCCUGGACAUCGGCAUCGCCUCCGUGGGCUGGGCCAUGGU |
| | | GGAGAUCGACGAGGAGGAGAACCCCAUCCGGCUGAUCGACCUGGGCGUGCGGGUGUUCGAGCGGGCCGAGGUGCCCAAGACCGGCGACUCC |
| | | CUGGCCAUGGCCCGGCGGCUGGCCCGGUCCGUGCGGCGGCUGACCCGGCGGCGGGCCCACCGGCUGCUGCGGGCCCGGCGGCUGCUGAAGC |
| | | GGGAGGGCGUGCUGCAGGCCGCCGACUUCGACGAGAACGGCCUGAUCAAGUCCCUGCCCAACACCCCCUGGCAGCUGCGGGCCGCCGCCCU |
| | | GGACCGGAAGCUGACCCCCCUGGAGUGGUCCGCCGUGCUGCUGCACCUGAUCAAGCACCGGGGCUACCUGUCCCAGCGGAAGAACGAGGGC |
| | | GAGACCGCCGACAAGGAGCUGGGCGCCCUGCUGAAGGGCGUGGCCAACAACGCCCACGCCCUGCAGACCGGCGACUUCCGGACCCCCGCCG |
| | | AGCUGGCCCUGAACAAGUUCGAGAAGGAGUCCGGCCACAUCCGGAACCAGCGGGGCGACUACUCCCACACCUUCUCCCGGAAGGACCUGCA |
| | | GGCCGAGCUGAUCCUGCUGUUCGAGAAGCAGAAGGAGUUCGGCAACCCCCACGUGUCCGGCGGCCUGAAGGAGGGCAUCGAGACCCUGCUG |
| | | AUGACCCAGCGGCCCGCCCUGUCCGGCGACGCCGUGCAGAAGAUGCUGGGCCACUGCACCUUCGAGCCCGCCGAGCCCAAGGCCGCCAAGA |
| | | ACACCUACACCGCCGAGCGGUUCAUCUGGCUGACCAAGCUGAACAACCUGCGGAUCCUGGAGCAGGGCUCCGAGCGGCCCCUGACCGACAC |
| | | CGAGCGGGCCACCCUGAUGGACGAGCCCUACCGGAAGUCCAAGCUGACCUACGCCCAGGCCCGGAAGCUGCUGGGCCUGGAGGACACCGCC |
| | | UUCUUCAAGGGCCUGCGGUACGGCAAGGACAACGCCGAGGCCUCCACCCUGAUGGAGAUGAAGGCCUACCACGCCAUCUCCCGGGCCCUGG |
| | | AGAAGGAGGGCCUGAAGGACAAGAAGUCCCCCCUGAACCUGUCCUCCGAGCUGCAGGACGAGAUCGGCACCGCCUUCUCCCUGUUCAAGAC |
| | | CGACGAGGACAUCACCGGCCGGCUGAAGGACCGGGUGCAGCCCGAGAUCCUGGAGGCCCUGCUGAAGCACAUCUCCUUCGACAAGUUCGUG |
| | | CAGAUCUCCCUGAAGGCCCUGCGGCGGAUCGUGCCCCUGAUGGAGCAGGGCAAGCGGUACGACGAGGCCUGCGCCGAGAUCUACGGCGACC |
| | | ACUACGGCAAGAAGAACACCGAGGAGAAGAUCUACCUGCCCCCCAUCCCCGCCGACGAGAUCCGGAACCCCGUGGUGCUGCGGGCCCUGUC |
| | | CCAGGCCCGGAAGGUGAUCAACGGCGUGGUGCGGCGGUACGGCUCCCCCGCCCGGAUCCACAUCGAGACCGCCCGGGAGGUGGGCAAGUCC |
| | | UUCAAGGACCGGAAGGAGAUCGAGAAGCGGCAGGAGGAGAACCGGAAGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGCUGUACGAGCAGCAGCACGGCAAGUGCCUGUACUCCGGCAAGGAGAU |
| | | CAACCUGGUGCGGCUGAACGAGAAGGGCUACGUGGAGAUCGACCACGCCCUGCCCUUCUCCCGGACCUGGGACGACUCCUUCAACAACAAG |
| | | GUGCUGGUGCUGGGCUCCGAGAACCAGAACAAGGGCAACCAGACCCCCUACGAGUACUUCAACGGCAAGGACAACUCCCGGGAGUGGCAGG |
| | | AGUUCAAGGCCCGGGUGGAGACCUCCCGGUUCCCCCGGUCCAAGAAGCAGCGGAUCCUGCUGCAGAAGUUCGACGAGGACGGCUUCAAGGA |
| | | GUGCAACCUGAACGACACCCGGUACGUGAACCGCUUCCUGUGCCAGUUCGUGGCCGACCACAUCCUGCUGACCGGCAAGGGCAAGCGGCGG |
| | | GUGUUCGCCUCCAACGGCCAGAUCACCAACCUGCUGCGGGGCUUCUGGGGCCUGCGGAAGGUGCGGGCCGAGAACGACCGGCACCACGCCC |
| | | UGGACGCCGUGGUGGUGGCCUGCUCCACCGUGGCCAUGCAGCAGAAGAUCACCCGGUUCGUGCGGUACAAGGAGAUGAACGCCUUCGACGG |
| | | CAAGACCAUCGACAAGGAGACCGGCAAGGUGCUGCACCAGAAGACCCACUUCCCCCAGCCCUGGGAGUUCUUCGCCCAGGAGGUGAUGAUC |
| | | CGGGUGUUCGGCAAGCCCGACGGCAAGCCCGAGUUCGAGGAGGCCGACACCCCCGAGAAGCUGCGGACCCUGCUGGCCGAGAAGCUGUCCU |
| | | CCCGGCCCGAGGCCGUGCACGAGUACGUGACCCCCCUGUUCGUGUCCCGGGCCCCCAACCGGAAGAUGUCCGGCGCCCACAAGGACACCCU |
| | | GCGGUCCGCCAAGCGGUUCGUGAAGCACAACGAGAAGAUCUCCGUGAAGCGGGUGUGGCUGACCGAGAUCAAGCUGGCCGACCUGGAGAAC |
| | | AUGGUGAACUACAAGAACGGCCGGGAGAUCGAGCUGUACGAGGCCCUGAAGGCCCGGCUGGAGGCCUACGGCGGCAACGCCAAGCAGGCCU |
| | | UCGACCCCAAGGACAACCCCUUCUACAAGAAGGGCGGCCAGCUGGUGAAGGCCGUGCGGGUGGAGAAGACCCAGGAGUCCGGCGUGCUGCU |
| | | GAACAAGAAGAACGCCUACACCAUCGCCGACAACGGCGACAUGGUGCGGGUGGACGUGUUCUGCAAGGUGGACAAGAAGGGCAAGAACCAG |
| | | UACUUCAUCGUGCCCAUCUACGCCUGGCAGGUGGCCGAGAACAUCCUGCCCGACAUCGACUGCAAGGGCUACCGGAUCGACGACUCCUACA |
| | | CCUUCUGCUUCUCCCUGCACAAGUACGACCUGAUCGCCUUCCAGAAGGACGAGAAGUCCAAGGUGGAGUUCGCCUACUACAUCAACUGCGA |
| | | CUCCUCCAACGGCCGGUUCUACCUGGCCUGGCACGACAAGGGCUCCAAGGAGCAGCAGUUCCGGAUCUCCACCCAGAACCUGGUGCUGAUC |
| | | CAGAAGUACCAGGUGAACGAGCUGGGCAAGGAGAUCCGGCCCUGCCGGCUGAAGAAGCGGCCCCCCGUGCGGUCCGGAAAGCGGACCGCCG |
| | | ACGGCUCCGGAGGAGGAAGCCCGAAGAAGAAGAGAAAGGUCUAG |
| 72 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 31 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | S)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | mRNA | LDKAGFIKRQLVETRQITKHVAQILDSRMNTKYDENDKLIREVKVITLKSKLVSDFRKDFQFYKVREINNYHHAHDAYLNAVVGTALIKKY |
| | sequence | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSSSGAAFKPNPINYILGLDIGIASVGWAMVEIDEEENPIRLIDLGVRVFERAEVPKTGDS |
| | | LAMARRLARSVRRLTRRRAHRLLRARRLLKREGVLQAADFDENGLIKSLPNTPWQLRAAALDRKLTPLEWSAVLLHLIKHRGYLSQRKNEG |
| | | ETADKELGALLKGVANNAHALQTGDFRTPAELALNKFEKESGHIRNQRGDYSHTFSRKDLQAELILLFEKQKEFGNPHVSGGLKEGIETLL |
| | | MTQRPALSGDAVQKMLGHCTFEPAEPKAAKNTYTAERFIWLTKLNNLRILEQGSERPLTDTERATLMDEPYRKSKLTYAQARKLLGLEDTA |
| | | FFKGLRYGKDNAEASTLMEMKAYHAISRALEKEGLKDKKSPLNLSSELQDEIGTAFSLFKTDEDITGRLKDRVQPEILEALLKHISFDKFV |
| | | QISLKALRRIVPLMEQGKRYDEACAEIYGDHYGKKNTEEKIYLPPIPADEIRNPVVLRALSQARKVINGVVRRYGSPARIHIETAREVGKS |
| | | FKDRKEIEKRQEENRKDREKAAAKFREYFPNFVGEPKSKDILKLRLYEQQHGKCLYSGKEINLVRLNEKGYVEIDHALPFSRTWDDSFNNK |
| | | VLVLGSENQNKGNQTPYEYFNGKDNSREWQEFKARVETSRFPRSKKQRILLQKFDEDGFKECNLNDTRYVNRFLCQFVADHILLTGKGKRR |
| | | VFASNGQITNLLRGFWGLRKVRAENDRHHALDAVVVACSTVAMQQKITRFVRYKEMNAFDGKTIDKETGKVLHQKTHFPQPWEFFAQEVMI |
| | | RVFGKPDGKPEFEEADTPEKLRTLLAEKLSSRPEAVHEYVTPLFVSRAPNRKMSGAHKDTLRSAKRFVKHNEKISVKRVWLTEIKLADLEN |
| | | MVNYKNGREIELYEALKARLEAYGGNAKQAFDPKDNPFYKKGGQLVKAVRVEKTQESGVLLNKKNAYTIADNGDMVRVDVFCKVDKKGKNQ |
| | | YFIVPIYAWQVAENILPDIDCKGYRIDDSYTFCFSLHKYDLIAFQKDEKSKVEFAYYINCDSSNGRFYLAWHDKGSKEQQFRISTQNLVLI |
| | | QKYQVNELGKEIRPCRLKKRPPVRSGKRTADGSGGGSPKKKRKV* |
| 73 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 21 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | T)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGCGGCGACGGCGCCCCCGAGUCCGCCACCGAGUCCGGCGGCACCUC |
| | | CACCGAGUCCGAGGGCUCCUCCUCCGGUGCCGCCUUCAAGCCCAACCCCAUCAACUACAUCCUGGGCCUGGACAUCGGCAUCGCCUCCGUG |
| | | GGCUGGGCCAUGGUGGAGAUCGACGAGGAGGAGAACCCCAUCCGGCUGAUCGACCUGGGCGUGCGGGUGUUCGAGCGGGCCGAGGUGCCCA |
| | | AGACCGGCGACUCCCUGGCCAUGGCCCGGCGGCUGGCCCGGUCCGUGCGGCGGCUGACCCGGCGGCGGGCCCACCGGCUGCUGCGGGCCCG |
| | | GCGGCUGCUGAAGCGGGAGGGCGUGCUGCAGGCCGCCGACUUCGACGAGAACGGCCUGAUCAAGUCCCUGCCCAACACCCCCUGGCAGCUG |
| | | CGGGCCGCCGCCCUGGACCGGAAGCUGACCCCCCUGGAGUGGUCCGCCGUGCUGCUGCACCUGAUCAAGCACCGGGGCUACCUGUCCCAGC |
| | | GGAAGAACGAGGGCGAGACCGCCGACAAGGAGCUGGGCGCCCUGCUGAAGGGCGUGGCCAACAACGCCCACGCCCUGCAGACCGGCGACUU |
| | | CCGGACCCCCGCCGAGCUGGCCCUGAACAAGUUCGAGAAGGAGUCCGGCCACAUCCGGAACCAGCGGGGCGACUACUCCCACACCUUCUCC |
| | | CGGAAGGACCUGCAGGCCGAGCUGAUCCUGCUGUUCGAGAAGCAGAAGGAGUUCGGCAACCCCCACGUGUCCGGCGGCCUGAAGGAGGGCA |
| | | UCGAGACCCUGCUGAUGACCCAGCGGCCCGCCCUGUCCGGCGACGCCGUGCAGAAGAUGCUGGGCCACUGCACCUUCGAGCCCGCCGAGCC |
| | | CAAGGCCGCCAAGAACACCUACACCGCCGAGCGGUUCAUCUGGCUGACCAAGCUGAACAACCUGCGGAUCCUGGAGCAGGGCUCCGAGCGG |
| | | CCCCUGACCGACACCGAGCGGGCCACCCUGAUGGACGAGCCCUACCGGAAGUCCAAGCUGACCUACGCCCAGGCCCGGAAGCUGCUGGGCC |
| | | UGGAGGACACCGCCUUCUUCAAGGGCCUGCGGUACGGCAAGGACAACGCCGAGGCCUCCACCCUGAUGGAGAUGAAGGCCUACCACGCCAU |
| | | CUCCCGGGCCCUGGAGAAGGAGGGCCUGAAGGACAAGAAGUCCCCCCUGAACCUGUCCUCCGAGCUGCAGGACGAGAUCGGCACCGCCUUC |
| | | UCCCUGUUCAAGACCGACGAGGACAUCACCGGCCGGCUGAAGGACCGGGUGCAGCCCGAGAUCCUGGAGGCCCUGCUGAAGCACAUCUCCU |
| | | UCGACAAGUUCGUGCAGAUCUCCCUGAAGGCCCUGCGGCGGAUCGUGCCCCUGAUGGAGCAGGGCAAGCGGUACGACGAGGCCUGCGCCGA |
| | | GAUCUACGGCGACCACUACGGCAAGAAGAACACCGAGGAGAAGAUCUACCUGCCCCCCAUCCCCGCCGACGAGAUCCGGAACCCCGUGGUG |
| | | CUGCGGGCCCUGUCCCAGGCCCGGAAGGUGAUCAACGGCGUGGUGCGGCGGUACGGCUCCCCCGCCCGGAUCCACAUCGAGACCGCCCGGG |
| | | AGGUGGGCAAGUCCUUCAAGGACCGGAAGGAGAUCGAGAAGCGGCAGGAGGAGAACCGGAAGGACCGGGAGAAGGCCGCCGCCAAGUUCCG |
| | | GGAGUACUUCCCCAACUUCGUGGGCGAGCCCAAGUCCAAGGACAUCCUGAAGCUGCGGCUGUACGAGCAGCAGCACGGCAAGUGCCUGUAC |
| | | UCCGGCAAGGAGAUCAACCUGGUGCGGCUGAACGAGAAGGGCUACGUGGAGAUCGACCACGCCCUGCCCUUCUCCCGGACCUGGGACGACU |
| | | CCUUCAACAACAAGGUGCUGGUGCUGGGCUCCGAGAACCAGAACAAGGGCAACCAGACCCCCUACGAGUACUUCAACGGCAAGGACAACUC |
| | | CCGGGAGUGGCAGGAGUUCAAGGCCCGGGUGGAGACCUCCCGGUUCCCCCGGUCCAAGAAGCAGCGGAUCCUGCUGCAGAAGUUCGACGAG |
| | | GACGGCUUCAAGGAGUGCAACCUGAACGACACCCGGUACGUGAACCGCUUCCUGUGCCAGUUCGUGGCCGACCACAUCCUGCUGACCGGCA |
| | | AGGGCAAGCGGCGGGUGUUCGCCUCCAACGGCCAGAUCACCAACCUGCUGCGGGGCUUCUGGGGCCUGCGGAAGGUGCGGGCCGAGAACGA |
| | | CCGGCACCACGCCCUGGACGCCGUGGUGGUGGCCUGCUCCACCGUGGCCAUGCAGCAGAAGAUCACCCGGUUCGUGCGGUACAAGGAGAUG |
| | | AACGCCUUCGACGGCAAGACCAUCGACAAGGAGACCGGCAAGGUGCUGCACCAGAAGACCCACUUCCCCCAGCCCUGGGAGUUCUUCGCCC |
| | | AGGAGGUGAUGAUCCGGGUGUUCGGCAAGCCCGACGGCAAGCCCGAGUUCGAGGAGGCCGACACCCCCGAGAAGCUGCGGACCCUGCUGGC |
| | | CGAGAAGCUGUCCUCCCGGCCCGAGGCCGUGCACGAGUACGUGACCCCCCUGUUCGUGUCCCGGGCCCCCAACCGGAAGAUGUCCGGCGCC |
| | | CACAAGGACACCCUGCGGUCCGCCAAGCGGUUCGUGAAGCACAACGAGAAGAUCUCCGUGAAGCGGGUGUGGCUGACCGAGAUCAAGCUGG |
| | | CCGACCUGGAGAACAUGGUGAACUACAAGAACGGCCGGGAGAUCGAGCUGUACGAGGCCCUGAAGGCCCGGCUGGAGGCCUACGGCGGCAA |
| | | CGCCAAGCAGGCCUUCGACCCCAAGGACAACCCCUUCUACAAGAAGGGCGGCCAGCUGGUGAAGGCCGUGCGGGUGGAGAAGACCCAGGAG |
| | | UCCGGCGUGCUGCUGAACAAGAAGAACGCCUACACCAUCGCCGACAACGGCGACAUGGUGCGGGUGGACGUGUUCUGCAAGGUGGACAAGA |
| | | AGGGCAAGAACCAGUACUUCAUCGUGCCCAUCUACGCCUGGCAGGUGGCCGAGAACAUCCUGCCCGACAUCGACUGCAAGGGCUACCGGAU |
| | | CGACGACUCCUACACCUUCUGCUUCUCCCUGCACAAGUACGACCUGAUCGCCUUCCAGAAGGACGAGAAGUCCAAGGUGGAGUUCGCCUAC |
| | | UACAUCAACUGCGACUCCUCCAACGGCCGGUUCUACCUGGCCUGGCACGACAAGGGCUCCAAGGAGCAGCAGUUCCGGAUCUCCACCCAGA |
| | | ACCUGGUGCUGAUCCAGAAGUACCAGGUGAACGAGCUGGGCAAGGAGAUCCGGCCCUGCCGGCUGAAGAAGCGGCCCCCCGUGCGGUCCGG |
| | | AAAGCGGACCGCCGACGGCUCCGGAGGAGGAAGCCCGAAGAAGAAGAGAAAGGUCAGCGAAAGCGCAACACCGGAAAGCGUCAGCGGAUGG |
| | | AGACUGUUCAAGAAGAUCAGCUAGCUAGCACCAGCCUCAAGAACACCCGAAUGGAGUCUCUAAGCUACAUAAUACCAACUUACACUUUACA |
| | | AAAUGUUGUCCCCCAAAAUGUAGCCAUUCGUAUCUGCUCCUAAUAAAAAGAAAGUUUCUUCACAUUCUCUCGAGAAAAAAAAAAAAUGGAA |
| | | AAAAAAAAAACGGAAAAAAAAAAAAGGUAAAAAAAAAAAAUAUAAAAAAAAAAAACAUAAAAAAAAAAAACGAAAAAAAAAAAACGUAAAA |
| | | AAAAAAAACUCAAAAAAAAAAAAGAUAAAAAAAAAAAACCUAAAAAAAAAAAAUGUAAAAAAAAAAAAGGGAAAAAAAAAAAACGCAAAAA |
| | | AAAAAAACACAAAAAAAAAAAAUGCAAAAAAAAAAAAUCGAAAAAAAAAAAAUCUAAAAAAAAAAAACGAAAAAAAAAAAACCCAAAAAAA |
| | | AAAAAGACAAAAAAAAAAAAUAGAAAAAAAAAAAAGUUAAAAAAAAAAAACUGAAAAAAAAAAAAUUUAAAAAAAAAAAAUCUAG |
| 74 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 21 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | tag | GACCUGGACAACCUGCUGGCCCAGAUCGGCGACCAGUACGCCGACCUGUUCCUGGCCGCCAAGAACCUGUCCGACGCCAUCCUGCUGUCCG |
| | HiBit | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | T)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCUCCUCCGGUGCCGCCUUCAAGCCCA |
| | | ACCCCAUCAACUACAUCCUGGGCCUGGACAUCGGCAUCGCCUCCGUGGGCUGGGCCAUGGUGGAGAUCGACGAGGAGGAGAACCCCAUCCG |
| | | GCUGAUCGACCUGGGCGUGCGGGUGUUCGAGCGGGCCGAGGUGCCCAAGACCGGCGACUCCCUGGCCAUGGCCCGGCGGCUGGCCCGGUCC |
| | | GUGCGGCGGCUGACCCGGCGGCGGGCCCACCGGCUGCUGCGGGCCCGGCGGCUGCUGAAGCGGGAGGGCGUGCUGCAGGCCGCCGACUUCG |
| | | ACGAGAACGGCCUGAUCAAGUCCCUGCCCAACACCCCCUGGCAGCUGCGGGCCGCCGCCCUGGACCGGAAGCUGACCCCCCUGGAGUGGUC |
| | | CGCCGUGCUGCUGCACCUGAUCAAGCACCGGGGCUACCUGUCCCAGCGGAAGAACGAGGGCGAGACCGCCGACAAGGAGCUGGGCGCCCUG |
| | | CUGAAGGGCGUGGCCAACAACGCCCACGCCCUGCAGACCGGCGACUUCCGGACCCCCGCCGAGCUGGCCCUGAACAAGUUCGAGAAGGAGU |
| | | CCGGCCACAUCCGGAACCAGCGGGGCGACUACUCCCACACCUUCUCCCGGAAGGACCUGCAGGCCGAGCUGAUCCUGCUGUUCGAGAAGCA |
| | | GAAGGAGUUCGGCAACCCCCACGUGUCCGGCGGCCUGAAGGAGGGCAUCGAGACCCUGCUGAUGACCCAGCGGCCCGCCCUGUCCGGCGAC |
| | | GCCGUGCAGAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGACGAGCCCUA |
| | | CCGGAAGUCCAAGCUGACCUACGCCCAGGCCCGGAAGCUGCUGGGCCUGGAGGACACCGCCUUCUUCAAGGGCCUGCGGUACGGCAAGGAC |
| | | AACGCCGAGGCCUCCACCCUGAUGGAGAUGAAGGCCUACCACGCCAUCUCCCGGGCCCUGGAGAAGGAGGGCCUGAAGGACAAGAAGUCCC |
| | | CCCUGAACCUGUCCUCCGAGCUGCAGGACGAGAUCGGCACCGCCUUCUCCCUGUUCAAGACCGACGAGGACAUCACCGGCCGGCUGAAGGA |
| | | CCGGGUGCAGCCCGAGAUCCUGGAGGCCCUGCUGAAGCACAUCUCCUUCGACAAGUUCGUGCAGAUCUCCCUGAAGGCCCUGCGGCGGAUC |
| | | GUGCCCCUGAUGGAGCAGGGCAAGCGGUACGACGAGGCCUGCGCCGAGAUCUACGGCGACCACUACGGCAAGAAGAACACCGAGGAGAAGA |
| | | UCUACCUGCCCCCCAUCCCCGCCGACGAGAUCCGGAACCCCGUGGUGCUGCGGGCCCUGUCCCAGGCCCGGAAGGUGAUCAACGGCGUGGU |
| | | GCGGCGGUACGGCUCCCCCGCCCGGAUCCACAUCGAGACCGCCCGGGAGGUGGGCAAGUCCUUCAAGGACCGGAAGGAGAUCGAGAAGCGG |
| | | CAGGAGGAGAACCGGAAGGACCGGGAGAAGGCCGCCGCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAAGGAGAUCAACCUGGUGCGGCUGAACGAGAAGGGCUA |
| | | CGUGGAGAUCGACCACGCCCUGCCCUUCUCCCGGACCUGGGACGACUCCUUCAACAACAAGGUGCUGGUGCUGGGCUCCGAGAACCAGAAC |
| | | AAGGGCAACCAGACCCCCUACGAGUACUUCAACGGCAAGGACAACUCCCGGGAGUGGCAGGAGUUCAAGGCCCGGGUGGAGACCUCCCGGU |
| | | UCCCCCGGUCCAAGAAGCAGCGGAUCCUGCUGCAGAAGUUCGACGAGGACGGCUUCAAGGAGUGCAACCUGAACGACACCCGGUACGUGAA |
| | | CCGCUUCCUGUGCCAGUUCGUGGCCGACCACAUCCUGCUGACCGGCAAGGGCAAGCGGCGGGUGUUCGCCUCCAACGGCCAGAUCACCAAC |
| | | CUGCUGCGGGGCUUCUGGGGCCUGCGGAAGGUGCGGGCCGAGAACGACCGGCACCACGCCCUGGACGCCGUGGUGGUGGCCUGCUCCACCG |
| | | UGGCCAUGCAGCAGAAGAUCACCCGGUUCGUGCGGUACAAGGAGAUGAACGCCUUCGACGGCAAGACCAUCGACAAGGAGACCGGCAAGGU |
| | | GCUGCACCAGAAGACCCACUUCCCCCAGCCCUGGGAGUUCUUCGCCCAGGAGGUGAUGAUCCGGGUGUUCGGCAAGCCCGACGGCAAGCCC |
| | | GAGUUCGAGGAGGCCGACACCCCCGAGAAGCUGCGGACCCUGCUGGCCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCGCCCACAAGGACACCCUGCGGUCCGCCAAGCGGUUCGUGAAGCACAA |
| | | CGAGAAGAUCUCCGUGAAGCGGGUGUGGCUGACCGAGAUCAAGCUGGCCGACCUGGAGAACAUGGUGAACUACAAGAACGGCCGGGAGAUC |
| | | GAGCUGUACGAGGCCCUGAAGGCCCGGCUGGAGGCCUACGGCGGCAACGCCAAGCAGGCCUUCGACCCCAAGGACAACCCCUUCUACAAGA |
| | | AGGGCGGCCAGCUGGUGAAGGCCGUGCGGGUGGAGAAGACCCAGGAGUCCGGCGUGCUGCUGAACAAGAAGAACGCCUACACCAUCGCCGA |
| | | CAACGGCGACAUGGUGCGGGUGGACGUGUUCUGCAAGGUGGACAAGAAGGGCAAGAACCAGUACUUCAUCGUGCCCAUCUACGCCUGGCAG |
| | | GUGGCCGAGAACAUCCUGCCCGACAUCGACUGCAAGGGCUACCGGAUCGACGACUCCUACACCUUCUGCUUCUCCCUGCACAAGUACGACC |
| | | UGAUCGCCUUCCAGAAGGACGAGAAGUCCAAGGUGGAGUUCGCCUACUACAUCAACUGCGACUCCUCCAACGGCCGGUUCUACCUGGCCUG |
| | | GCACGACAAGGGCUCCAAGGAGCAGCAGUUCCGGAUCUCCACCCAGAACCUGGUGCUGAUCCAGAAGUACCAGGUGAACGAGCUGGGCAAG |
| | | GAGAUCCGGCCCUGCCGGCUGAAGAAGCGGCCCCCCGUGCGGUCCGGAAAGCGGACCGCCGACGGCUCCGGAGGAGGAAGCCCGAAGAAGA |
| | | AGAGAAAGGUCAGCGAAAGCGCAACACCGGAAAGCGUCAGCGGAUGGAGACUGUUCAAGAAGAUCAGCUAG |
| 75 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 21 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | T)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATESGGTSTESEGSSSGAAFKPNPINYILGLDIGIASVGWAMVEIDEEENPIRLIDLGVRVFERAEVPKTGDSLAMARRLARS |
| | | VRRLTRRRAHRLLRARRLLKREGVLQAADFDENGLIKSLPNTPWQLRAAALDRKLTPLEWSAVLLHLIKHRGYLSQRKNEGETADKELGAL |
| | | LKGVANNAHALQTGDFRTPAELALNKFEKESGHIRNQRGDYSHTFSRKDLQAELILLFEKQKEFGNPHVSGGLKEGIETLLMTQRPALSGD |
| | | AVQKMLGHCTFEPAEPKAAKNTYTAERFIWLTKLNNLRILEQGSERPLTDTERATLMDEPYRKSKLTYAQARKLLGLEDTAFFKGLRYGKD |
| | | NAEASTLMEMKAYHAISRALEKEGLKDKKSPLNLSSELQDEIGTAFSLFKTDEDITGRLKDRVQPEILEALLKHISFDKFVQISLKALRRI |
| | | VPLMEQGKRYDEACAEIYGDHYGKKNTEEKIYLPPIPADEIRNPVVLRALSQARKVINGVVRRYGSPARIHIETAREVGKSFKDRKEIEKR |
| | | QEENRKDREKAAAKFREYFPNFVGEPKSKDILKLRLYEQQHGKCLYSGKEINLVRLNEKGYVEIDHALPFSRTWDDSFNNKVLVLGSENQN |
| | | KGNQTPYEYFNGKDNSREWQEFKARVETSRFPRSKKQRILLQKFDEDGFKECNLNDTRYVNRFLCQFVADHILLTGKGKRRVFASNGQITN |
| | | LLRGFWGLRKVRAENDRHHALDAVVVACSTVAMQQKITRFVRYKEMNAFDGKTIDKETGKVLHQKTHFPQPWEFFAQEVMIRVFGKPDGKP |
| | | EFEEADTPEKLRTLLAEKLSSRPEAVHEYVTPLFVSRAPNRKMSGAHKDTLRSAKRFVKHNEKISVKRVWLTEIKLADLENMVNYKNGREI |
| | | ELYEALKARLEAYGGNAKQAFDPKDNPFYKKGGQLVKAVRVEKTQESGVLLNKKNAYTIADNGDMVRVDVFCKVDKKGKNQYFIVPIYAWQ |
| | | VAENILPDIDCKGYRIDDSYTFCFSLHKYDLIAFQKDEKSKVEFAYYINCDSSNGRFYLAWHDKGSKEQQFRISTQNLVLIQKYQVNELGK |
| | | EIRPCRLKKRPPVRSGKRTADGSGGGSPKKKRKVSESATPESVSGWRLFKKIS* |
| 76 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 21 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | T)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGAGUCCGGCGGCACCUCCACCGAGUCCGAGGGCUCCUCCUCCGGUGCCGCCUUCAAGCCCA |
| | | ACCCCAUCAACUACAUCCUGGGCCUGGACAUCGGCAUCGCCUCCGUGGGCUGGGCCAUGGUGGAGAUCGACGAGGAGGAGAACCCCAUCCG |
| | | GCUGAUCGACCUGGGCGUGCGGGUGUUCGAGCGGGCCGAGGUGCCCAAGACCGGCGACUCCCUGGCCAUGGCCCGGCGGCUGGCCCGGUCC |
| | | GUGCGGCGGCUGACCCGGCGGCGGGCCCACCGGCUGCUGCGGGCCCGGCGGCUGCUGAAGCGGGAGGGCGUGCUGCAGGCCGCCGACUUCG |
| | | ACGAGAACGGCCUGAUCAAGUCCCUGCCCAACACCCCCUGGCAGCUGCGGGCCGCCGCCCUGGACCGGAAGCUGACCCCCCUGGAGUGGUC |
| | | CGCCGUGCUGCUGCACCUGAUCAAGCACCGGGGCUACCUGUCCCAGCGGAAGAACGAGGGCGAGACCGCCGACAAGGAGCUGGGCGCCCUG |
| | | CUGAAGGGCGUGGCCAACAACGCCCACGCCCUGCAGACCGGCGACUUCCGGACCCCCGCCGAGCUGGCCCUGAACAAGUUCGAGAAGGAGU |
| | | CCGGCCACAUCCGGAACCAGCGGGGCGACUACUCCCACACCUUCUCCCGGAAGGACCUGCAGGCCGAGCUGAUCCUGCUGUUCGAGAAGCA |
| | | GAAGGAGUUCGGCAACCCCCACGUGUCCGGCGGCCUGAAGGAGGGCAUCGAGACCCUGCUGAUGACCCAGCGGCCCGCCCUGUCCGGCGAC |
| | | GCCGUGCAGAAGAUGCUGGGCCACUGCACCUUCGAGCCCGCCGAGCCCAAGGCCGCCAAGAACACCUACACCGCCGAGCGGUUCAUCUGGC |
| | | UGACCAAGCUGAACAACCUGCGGAUCCUGGAGCAGGGCUCCGAGCGGCCCCUGACCGACACCGAGCGGGCCACCCUGAUGGACGAGCCCUA |
| | | CCGGAAGUCCAAGCUGACCUACGCCCAGGCCCGGAAGCUGCUGGGCCUGGAGGACACCGCCUUCUUCAAGGGCCUGCGGUACGGCAAGGAC |
| | | AACGCCGAGGCCUCCACCCUGAUGGAGAUGAAGGCCUACCACGCCAUCUCCCGGGCCCUGGAGAAGGAGGGCCUGAAGGACAAGAAGUCCC |
| | | CCCUGAACCUGUCCUCCGAGCUGCAGGACGAGAUCGGCACCGCCUUCUCCCUGUUCAAGACCGACGAGGACAUCACCGGCCGGCUGAAGGA |
| | | CCGGGUGCAGCCCGAGAUCCUGGAGGCCCUGCUGAAGCACAUCUCCUUCGACAAGUUCGUGCAGAUCUCCCUGAAGGCCCUGCGGCGGAUC |
| | | GUGCCCCUGAUGGAGCAGGGCAAGCGGUACGACGAGGCCUGCGCCGAGAUCUACGGCGACCACUACGGCAAGAAGAACACCGAGGAGAAGA |
| | | UCUACCUGCCCCCCAUCCCCGCCGACGAGAUCCGGAACCCCGUGGUGCUGCGGGCCCUGUCCCAGGCCCGGAAGGUGAUCAACGGCGUGGU |
| | | GCGGCGGUACGGCUCCCCCGCCCGGAUCCACAUCGAGACCGCCCGGGAGGUGGGCAAGUCCUUCAAGGACCGGAAGGAGAUCGAGAAGCGG |
| | | CAGGAGGAGAACCGGAAGGACCGGGAGAAGGCCGCCGCCAAGUUCCGGGAGUACUUCCCCAACUUCGUGGGCGAGCCCAAGUCCAAGGACA |
| | | UCCUGAAGCUGCGGCUGUACGAGCAGCAGCACGGCAAGUGCCUGUACUCCGGCAAGGAGAUCAACCUGGUGCGGCUGAACGAGAAGGGCUA |
| | | CGUGGAGAUCGACCACGCCCUGCCCUUCUCCCGGACCUGGGACGACUCCUUCAACAACAAGGUGCUGGUGCUGGGCUCCGAGAACCAGAAC |
| | | AAGGGCAACCAGACCCCCUACGAGUACUUCAACGGCAAGGACAACUCCCGGGAGUGGCAGGAGUUCAAGGCCCGGGUGGAGACCUCCCGGU |
| | | UCCCCCGGUCCAAGAAGCAGCGGAUCCUGCUGCAGAAGUUCGACGAGGACGGCUUCAAGGAGUGCAACCUGAACGACACCCGGUACGUGAA |
| | | CCGCUUCCUGUGCCAGUUCGUGGCCGACCACAUCCUGCUGACCGGCAAGGGCAAGCGGCGGGUGUUCGCCUCCAACGGCCAGAUCACCAAC |
| | | CUGCUGCGGGGCUUCUGGGGCCUGCGGAAGGUGCGGGCCGAGAACGACCGGCACCACGCCCUGGACGCCGUGGUGGUGGCCUGCUCCACCG |
| | | UGGCCAUGCAGCAGAAGAUCACCCGGUUCGUGCGGUACAAGGAGAUGAACGCCUUCGACGGCAAGACCAUCGACAAGGAGACCGGCAAGGU |
| | | GCUGCACCAGAAGACCCACUUCCCCCAGCCCUGGGAGUUCUUCGCCCAGGAGGUGAUGAUCCGGGUGUUCGGCAAGCCCGACGGCAAGCCC |
| | | GAGUUCGAGGAGGCCGACACCCCCGAGAAGCUGCGGACCCUGCUGGCCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCGCCCACAAGGACACCCUGCGGUCCGCCAAGCGGUUCGUGAAGCACAA |
| | | CGAGAAGAUCUCCGUGAAGCGGGUGUGGCUGACCGAGAUCAAGCUGGCCGACCUGGAGAACAUGGUGAACUACAAGAACGGCCGGGAGAUC |
| | | GAGCUGUACGAGGCCCUGAAGGCCCGGCUGGAGGCCUACGGCGGCAACGCCAAGCAGGCCUUCGACCCCAAGGACAACCCCUUCUACAAGA |
| | | AGGGCGGCCAGCUGGUGAAGGCCGUGCGGGUGGAGAAGACCCAGGAGUCCGGCGUGCUGCUGAACAAGAAGAACGCCUACACCAUCGCCGA |
| | | CAACGGCGACAUGGUGCGGGUGGACGUGUUCUGCAAGGUGGACAAGAAGGGCAAGAACCAGUACUUCAUCGUGCCCAUCUACGCCUGGCAG |
| | | GUGGCCGAGAACAUCCUGCCCGACAUCGACUGCAAGGGCUACCGGAUCGACGACUCCUACACCUUCUGCUUCUCCCUGCACAAGUACGACC |
| | | UGAUCGCCUUCCAGAAGGACGAGAAGUCCAAGGUGGAGUUCGCCUACUACAUCAACUGCGACUCCUCCAACGGCCGGUUCUACCUGGCCUG |
| | | GCACGACAAGGGCUCCAAGGAGCAGCAGUUCCGGAUCUCCACCCAGAACCUGGUGCUGAUCCAGAAGUACCAGGUGAACGAGCUGGGCAAG |
| | | GAGAUCCGGCCCUGCCGGCUGAAGAAGCGGCCCCCCGUGCGGUCCGGAAAGCGGACCGCCGACGGCUCCGGAGGAGGAAGCCCGAAGAAGA |
| | | AGAGAAAGGUCUAG |
| 77 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 21 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | T)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | amino | LDKAGFIKRQLVETRQITKHVAQILDSRMNTKYDENDKLIREVKVITLKSKLVSDFRKDFQFYKVREINNYHHAHDAYLNAVVGTALIKKY |
| | acid | PKLESEFVYGDYKVYDVRKMIAKSEQEIGKATAKYFFYSNIMNFFKTEITLANGEIRKRPLIETNGETGEIVWDKGRDFATVRKVLSMPQV |
| | sequence | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSSSGAAFKPNPINYILGLDIGIASVGWAMVEIDEEENPIRLIDLGVRVFERAEVPKTGDSLAMARRLARS |
| | | VRRLTRRRAHRLLRARRLLKREGVLQAADFDENGLIKSLPNTPWQLRAAALDRKLTPLEWSAVLLHLIKHRGYLSQRKNEGETADKELGAL |
| | | LKGVANNAHALQTGDFRTPAELALNKFEKESGHIRNQRGDYSHTFSRKDLQAELILLFEKQKEFGNPHVSGGLKEGIETLLMTQRPALSGD |
| | | AVQKMLGHCTFEPAEPKAAKNTYTAERFIWLTKLNNLRILEQGSERPLTDTERATLMDEPYRKSKLTYAQARKLLGLEDTAFFKGLRYGKD |
| | | NAEASTLMEMKAYHAISRALEKEGLKDKKSPLNLSSELQDEIGTAFSLFKTDEDITGRLKDRVQPEILEALLKHISFDKFVQISLKALRRI |
| | | VPLMEQGKRYDEACAEIYGDHYGKKNTEEKIYLPPIPADEIRNPVVLRALSQARKVINGVVRRYGSPARIHIETAREVGKSFKDRKEIEKR |
| | | QEENRKDREKAAAKFREYFPNFVGEPKSKDILKLRLYEQQHGKCLYSGKEINLVRLNEKGYVEIDHALPFSRTWDDSFNNKVLVLGSENQN |
| | | KGNQTPYEYFNGKDNSREWQEFKARVETSRFPRSKKQRILLQKFDEDGFKECNLNDTRYVNRFLCQFVADHILLTGKGKRRVFASNGQITN |
| | | LLRGFWGLRKVRAENDRHHALDAVVVACSTVAMQQKITRFVRYKEMNAFDGKTIDKETGKVLHQKTHFPQPWEFFAQEVMIRVFGKPDGKP |
| | | EFEEADTPEKLRTLLAEKLSSRPEAVHEYVTPLFVSRAPNRKMSGAHKDTLRSAKRFVKHNEKISVKRVWLTEIKLADLENMVNYKNGREI |
| | | ELYEALKARLEAYGGNAKQAFDPKDNPFYKKGGQLVKAVRVEKTQESGVLLNKKNAYTIADNGDMVRVDVFCKVDKKGKNQYFIVPIYAWQ |
| | | VAENILPDIDCKGYRIDDSYTFCFSLHKYDLIAFQKDEKSKVEFAYYINCDSSNGRFYLAWHDKGSKEQQFRISTQNLVLIQKYQVNELGK |
| | | EIRPCRLKKRPPVRSGKRTADGSGGGSPKKKRKV* |
| 78 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 11 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | (mRNA | UCCAUGAUCAAGCGGUACGACGAGCACCACCAGGACCUGACCCUGCUGAAGGCCCUGGUGCGGCAGCAGCUGCCCGAGAAGUACAAGGAGA |
| | U)- | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | mRNA | GGAGAAGAUGGACGGCACCGAGGAGCUGCUGGUGAAGCUGAACCGGGAGGACCUGCUGCGGAAGCAGCGGACCUUCGACAACGGCUCCAUC |
| | sequence | CCCCACCAGAUCCACCUGGGCGAGCUGCACGCCAUCCUGCGGCGGCAGGAGGACUUCUACCCCUUCCUGAAGGACAACCGGGAGAAGAUCG |
| | | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | | GACCAUCACCCCCUGGAACUUCGAGGAGGUGGUGGACAAGGGCGCCUCCGCCCAGUCCUUCAUCGAGCGGAUGACCAACUUCGACAAGAAC |
| | | CUGCCCAACGAGAAGGUGCUGCCCAAGCACUCCCUGCUGUACGAGUACUUCACCGUGUACAACGAGCUGACCAAGGUGAAGUACGUGACCG |
| | | AGGGCAUGCGGAAGCCCGCCUUCCUGUCCGGCGAGCAGAAGAAGGCCAUCGUGGACCUGCUGUUCAAGACCAACCGGAAGGUGACCGUGAA |
| | | GCAGCUGAAGGAGGACUACUUCAAGAAGAUCGAGUGCUUCGACUCCGUGGAGAUCUCCGGCGUGGAGGACCGGUUCAACGCCUCCCUGGGC |
| | | ACCUACCACGACCUGCUGAAGAUCAUCAAGGACAAGGACUUCCUGGACAACGAGGAGAACGAGGACAUCCUGGAGGACAUCGUGCUGACCC |
| | | UGACCCUGUUCGAGGACCGGGAGAUGAUCGAGGAGCGGCUGAAGACCUACGCCCACCUGUUCGACGACAAGGUGAUGAAGCAGCUGAAGCG |
| | | GCGGCGGUACACCGGCUGGGGCCGGCUGUCCCGGAAGCUGAUCAACGGCAUCCGGGACAAGCAGUCCGGCAAGACCAUCCUGGACUUCCUG |
| | | AAGUCCGACGGCUUCGCCAACCGGAACUUCAUGCAGCUGAUCCACGACGACUCCCUGACCUUCAAGGAGGACAUCCAGAAGGCCCAGGUGU |
| | | CCGGCCAGGGCGACUCCCUGCACGAGCACAUCGCCAACCUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCGAGAUGGCCCGGGAGAACCAGACCACCCAGAAGGGCCAG |
| | | AAGAACUCCCGGGAGCGGAUGAAGCGGAUCGAGGAGGGCAUCAAGGAGCUGGGCUCCCAGAUCCUGAAGGAGCACCCCGUGGAGAACACCC |
| | | AGCUGCAGAACGAGAAGCUGUACCUGUACUACCUGCAGAACGGCCGGGACAUGUACGUGGACCAGGAGCUGGACAUCAACCGGCUGUCCGA |
| | | CUACGACGUGGACCACAUCGUGCCCCAGUCCUUCCUGAAGGACGACUCCAUCGACAACAAGGUGCUGACCCGGUCCGACAAGAACCGGGGC |
| | | AAGUCCGACAACGUGCCCUCCGAGGAGGUGGUGAAGAAGAUGAAGAACUACUGGCGGCAGCUGCUGAACGCCAAGCUGAUCACCCAGCGGA |
| | | AGUUCGACAACCUGACCAAGGCCGAGCGGGGCGGCCUGUCCGAGCUGGACAAGGCCGGCUUCAUCAAGCGGCAGCUGGUGGAGACCCGGCA |
| | | GAUCACCAAGCACGUGGCCCAGAUCCUGGACUCCCGGAUGAACACCAAGUACGACGAGAACGACAAGCUGAUCCGGGAGGUGAAGGUGAUC |
| | | ACCCUGAAGUCCAAGCUGGUGUCCGACUUCCGGAAGGACUUCCAGUUCUACAAGGUGCGGGAGAUCAACAACUACCACCACGCCCACGACG |
| | | CCUACCUGAACGCCGUGGUGGGCACCGCCCUGAUCAAGAAGUACCCCAAGCUGGAGUCCGAGUUCGUGUACGGCGACUACAAGGUGUACGA |
| | | CGUGCGGAAGAUGAUCGCCAAGUCCGAGCAGGAGAUCGGCAAGGCCACCGCCAAGUACUUCUUCUACUCCAACAUCAUGAACUUCUUCAAG |
| | | ACCGAGAUCACCCUGGCCAACGGCGAGAUCCGGAAGCGGCCCCUGAUCGAGACCAACGGCGAGACCGGCGAGAUCGUGUGGGACAAGGGCC |
| | | GGGACUUCGCCACCGUGCGGAAGGUGCUGUCCAUGCCCCAGGUGAACAUCGUGAAGAAGACCGAGGUGCAGACCGGCGGCUUCUCCAAGGA |
| | | GUCCAUCCUGCCCAAGCGGAACUCCGACAAGCUGAUCGCCCGGAAGAAGGACUGGGACCCCAAGAAGUACGGCGGCUUCGACUCCCCCACC |
| | | GUGGCCUACUCCGUGCUGGUGGUGGCCAAGGUGGAGAAGGGCAAGUCCAAGAAGCUGAAGUCCGUGAAGGAGCUGCUGGGCAUCACCAUCA |
| | | UGGAGCGGUCCUCCUUCGAGAAGAACCCCAUCGACUUCCUGGAGGCCAAGGGCUACAAGGAGGUGAAGAAGGACCUGAUCAUCAAGCUGCC |
| | | CAAGUACUCCCUGUUCGAGCUGGAGAACGGCCGGAAGCGGAUGCUGGCCUCCGCCGGCGAGCUGCAGAAGGGCAACGAGCUGGCCCUGCCC |
| | | UCCAAGUACGUGAACUUCCUGUACCUGGCCUCCCACUACGAGAAGCUGAAGGGCUCCCCCGAGGACAACGAGCAGAAGCAGCUGUUCGUGG |
| | | AGCAGCACAAGCACUACCUGGACGAGAUCAUCGAGCAGAUCUCCGAGUUCUCCAAGCGGGUGAUCCUGGCCGACGCCAACCUGGACAAGGU |
| | | GCUGUCCGCCUACAACAAGCACCGGGACAAGCCCAUCCGGGAGCAGGCCGAGAACAUCAUCCACCUGUUCACCCUGACCAACCUGGGCGCC |
| | | CCCGCCGCCUUCAAGUACUUCGACACCACCAUCGACAAGAAGCGGUACACCUCCACCAAGGAGGUGCUGGACGCCACCCUGAUCCACCAGU |
| | | CCAUCACCGGCCUGUACGAGACCCGGAUCGACCUGUCCCAGCUGGGGGGCGACGGCGCCCCCGAGUCCGCCACCGGCUCCUCCUCCGGUGC |
| | | CGCCUUCAAGCCCAACCCCAUCAACUACAUCCUGGGCCUGGACAUCGGCAUCGCCUCCGUGGGCUGGGCCAUGGUGGAGAUCGACGAGGAG |
| | | GAGAACCCCAUCCGGCUGAUCGACCUGGGCGUGCGGGUGUUCGAGCGGGCCGAGGUGCCCAAGACCGGCGACUCCCUGGCCAUGGCCCGGC |
| | | GGCUGGCCCGGUCCGUGCGGCGGCUGACCCGGCGGCGGGCCCACCGGCUGCUGCGGGCCCGGCGGCUGCUGAAGCGGGAGGGCGUGCUGCA |
| | | GGCCGCCGACUUCGACGAGAACGGCCUGAUCAAGUCCCUGCCCAACACCCCCUGGCAGCUGCGGGCCGCCGCCCUGGACCGGAAGCUGACC |
| | | CCCCUGGAGUGGUCCGCCGUGCUGCUGCACCUGAUCAAGCACCGGGGCUACCUGUCCCAGCGGAAGAACGAGGGCGAGACCGCCGACAAGG |
| | | AGCUGGGCGCCCUGCUGAAGGGCGUGGCCAACAACGCCCACGCCCUGCAGACCGGCGACUUCCGGACCCCCGCCGAGCUGGCCCUGAACAA |
| | | GUUCGAGAAGGAGUCCGGCCACAUCCGGAACCAGCGGGGCGACUACUCCCACACCUUCUCCCGGAAGGACCUGCAGGCCGAGCUGAUCCUG |
| | | CUGUUCGAGAAGCAGAAGGAGUUCGGCAACCCCCACGUGUCCGGCGGCCUGAAGGAGGGCAUCGAGACCCUGCUGAUGACCCAGCGGCCCG |
| | | CCCUGUCCGGCGACGCCGUGCAGAAGAUGCUGGGCCACUGCACCUUCGAGCCCGCCGAGCCCAAGGCCGCCAAGAACACCUACACCGCCGA |
| | | GCGGUUCAUCUGGCUGACCAAGCUGAACAACCUGCGGAUCCUGGAGCAGGGCUCCGAGCGGCCCCUGACCGACACCGAGCGGGCCACCCUG |
| | | AUGGACGAGCCCUACCGGAAGUCCAAGCUGACCUACGCCCAGGCCCGGAAGCUGCUGGGCCUGGAGGACACCGCCUUCUUCAAGGGCCUGC |
| | | GGUACGGCAAGGACAACGCCGAGGCCUCCACCCUGAUGGAGAUGAAGGCCUACCACGCCAUCUCCCGGGCCCUGGAGAAGGAGGGCCUGAA |
| | | GGACAAGAAGUCCCCCCUGAACCUGUCCUCCGAGCUGCAGGACGAGAUCGGCACCGCCUUCUCCCUGUUCAAGACCGACGAGGACAUCACC |
| | | GGCCGGCUGAAGGACCGGGUGCAGCCCGAGAUCCUGGAGGCCCUGCUGAAGCACAUCUCCUUCGACAAGUUCGUGCAGAUCUCCCUGAAGG |
| | | CCCUGCGGCGGAUCGUGCCCCUGAUGGAGCAGGGCAAGCGGUACGACGAGGCCUGCGCCGAGAUCUACGGCGACCACUACGGCAAGAAGAA |
| | | CACCGAGGAGAAGAUCUACCUGCCCCCCAUCCCCGCCGACGAGAUCCGGAACCCCGUGGUGCUGCGGGCCCUGUCCCAGGCCCGGAAGGUG |
| | | AUCAACGGCGUGGUGCGGCGGUACGGCUCCCCCGCCCGGAUCCACAUCGAGACCGCCCGGGAGGUGGGCAAGUCCUUCAAGGACCGGAAGG |
| | | AGAUCGAGAAGCGGCAGGAGGAGAACCGGAAGGACCGGGAGAAGGCCGCCGCCAAGUUCCGGGAGUACUUCCCCAACUUCGUGGGCGAGCC |
| | | CAAGUCCAAGGACAUCCUGAAGCUGCGGCUGUACGAGCAGCAGCACGGCAAGUGCCUGUACUCCGGCAAGGAGAUCAACCUGGUGCGGCUG |
| | | AACGAGAAGGGCUACGUGGAGAUCGACCACGCCCUGCCCUUCUCCCGGACCUGGGACGACUCCUUCAACAACAAGGUGCUGGUGCUGGGCU |
| | | CCGAGAACCAGAACAAGGGCAACCAGACCCCCUACGAGUACUUCAACGGCAAGGACAACUCCCGGGAGUGGCAGGAGUUCAAGGCCCGGGU |
| | | GGAGACCUCCCGGUUCCCCCGGUCCAAGAAGCAGCGGAUCCUGCUGCAGAAGUUCGACGAGGACGGCUUCAAGGAGUGCAACCUGAACGAC |
| | | ACCCGGUACGUGAACCGCUUCCUGUGCCAGUUCGUGGCCGACCACAUCCUGCUGACCGGCAAGGGCAAGCGGCGGGUGUUCGCCUCCAACG |
| | | GCCAGAUCACCAACCUGCUGCGGGGCUUCUGGGGCCUGCGGAAGGUGCGGGCCGAGAACGACCGGCACCACGCCCUGGACGCCGUGGUGGU |
| | | GGCCUGCUCCACCGUGGCCAUGCAGCAGAAGAUCACCCGGUUCGUGCGGUACAAGGAGAUGAACGCCUUCGACGGCAAGACCAUCGACAAG |
| | | GAGACCGGCAAGGUGCUGCACCAGAAGACCCACUUCCCCCAGCCCUGGGAGUUCUUCGCCCAGGAGGUGAUGAUCCGGGUGUUCGGCAAGC |
| | | CCGACGGCAAGCCCGAGUUCGAGGAGGCCGACACCCCCGAGAAGCUGCGGACCCUGCUGGCCGAGAAGCUGUCCUCCCGGCCCGAGGCCGU |
| | | GCACGAGUACGUGACCCCCCUGUUCGUGUCCCGGGCCCCCAACCGGAAGAUGUCCGGCGCCCACAAGGACACCCUGCGGUCCGCCAAGCGG |
| | | UUCGUGAAGCACAACGAGAAGAUCUCCGUGAAGCGGGUGUGGCUGACCGAGAUCAAGCUGGCCGACCUGGAGAACAUGGUGAACUACAAGA |
| | | ACGGCCGGGAGAUCGAGCUGUACGAGGCCCUGAAGGCCCGGCUGGAGGCCUACGGCGGCAACGCCAAGCAGGCCUUCGACCCCAAGGACAA |
| | | CCCCUUCUACAAGAAGGGCGGCCAGCUGGUGAAGGCCGUGCGGGUGGAGAAGACCCAGGAGUCCGGCGUGCUGCUGAACAAGAAGAACGCC |
| | | UACACCAUCGCCGACAACGGCGACAUGGUGCGGGUGGACGUGUUCUGCAAGGUGGACAAGAAGGGCAAGAACCAGUACUUCAUCGUGCCCA |
| | | UCUACGCCUGGCAGGUGGCCGAGAACAUCCUGCCCGACAUCGACUGCAAGGGCUACCGGAUCGACGACUCCUACACCUUCUGCUUCUCCCU |
| | | GCACAAGUACGACCUGAUCGCCUUCCAGAAGGACGAGAAGUCCAAGGUGGAGUUCGCCUACUACAUCAACUGCGACUCCUCCAACGGCCGG |
| | | UUCUACCUGGCCUGGCACGACAAGGGCUCCAAGGAGCAGCAGUUCCGGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGCCCCCCGUGCGGUCCGGAAAGCGGACCGCCGACGGCUCCGGAGGAGG |
| | | AAGCCCGAAGAAGAAGAGAAAGGUCAGCGAAAGCGCAACACCGGAAAGCGUCAGCGGAUGGAGACUGUUCAAGAAGAUCAGCUAGCUAGCA |
| | | CCAGCCUCAAGAACACCCGAAUGGAGUCUCUAAGCUACAUAAUACCAACUUACACUUUACAAAAUGUUGUCCCCCAAAAUGUAGCCAUUCG |
| | | UAUCUGCUCCUAAUAAAAAGAAAGUUUCUUCACAUUCUCUCGAGAAAAAAAAAAAAUGGAAAAAAAAAAAACGGAAAAAAAAAAAAGGUAA |
| | | AAAAAAAAAAUAUAAAAAAAAAAAACAUAAAAAAAAAAAACGAAAAAAAAAAAACGUAAAAAAAAAAAACUCAAAAAAAAAAAAGAUAAAA |
| | | AAAAAAAACCUAAAAAAAAAAAAUGUAAAAAAAAAAAAGGGAAAAAAAAAAAACGCAAAAAAAAAAAACACAAAAAAAAAAAAUGCAAAAA |
| | | AAAAAAAUCGAAAAAAAAAAAAUCUAAAAAAAAAAAACGAAAAAAAAAAAACCCAAAAAAAAAAAAGACAAAAAAAAAAAAUAGAAAAAAA |
| | | AAAAAGUUAAAAAAAAAAAACUGAAAAAAAAAAAAUUUAAAAAAAAAAAAUCUAG |
| 79 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 11 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag | ACAUCCUGCGGGUGAACACCGAGAUCACCAAGGCCCCCCUGUCCGCCUCCAUGAUCAAGCGGUACGACGAGCACCACCAGGACCUGACCCU |
| | (mRNA | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | U)-ORF | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | sequence | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGGCUCCUCCUCCGGUGCCGCCUUCAAGCCCAACCCCAUCAACUACAUCCUGGGCCUGGACA |
| | | UCGGCAUCGCCUCCGUGGGCUGGGCCAUGGUGGAGAUCGACGAGGAGGAGAACCCCAUCCGGCUGAUCGACCUGGGCGUGCGGGUGUUCGA |
| | | GCGGGCCGAGGUGCCCAAGACCGGCGACUCCCUGGCCAUGGCCCGGCGGCUGGCCCGGUCCGUGCGGCGGCUGACCCGGCGGCGGGCCCAC |
| | | CGGCUGCUGCGGGCCCGGCGGCUGCUGAAGCGGGAGGGCGUGCUGCAGGCCGCCGACUUCGACGAGAACGGCCUGAUCAAGUCCCUGCCCA |
| | | ACACCCCCUGGCAGCUGCGGGCCGCCGCCCUGGACCGGAAGCUGACCCCCCUGGAGUGGUCCGCCGUGCUGCUGCACCUGAUCAAGCACCG |
| | | GGGCUACCUGUCCCAGCGGAAGAACGAGGGCGAGACCGCCGACAAGGAGCUGGGCGCCCUGCUGAAGGGCGUGGCCAACAACGCCCACGCC |
| | | CUGCAGACCGGCGACUUCCGGACCCCCGCCGAGCUGGCCCUGAACAAGUUCGAGAAGGAGUCCGGCCACAUCCGGAACCAGCGGGGCGACU |
| | | ACUCCCACACCUUCUCCCGGAAGGACCUGCAGGCCGAGCUGAUCCUGCUGUUCGAGAAGCAGAAGGAGUUCGGCAACCCCCACGUGUCCGG |
| | | CGGCCUGAAGGAGGGCAUCGAGACCCUGCUGAUGACCCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCAUCUGGCUGACCAAGCUGAACAACCUGCGGAUCCUGG |
| | | AGCAGGGCUCCGAGCGGCCCCUGACCGACACCGAGCGGGCCACCCUGAUGGACGAGCCCUACCGGAAGUCCAAGCUGACCUACGCCCAGGC |
| | | CCGGAAGCUGCUGGGCCUGGAGGACACCGCCUUCUUCAAGGGCCUGCGGUACGGCAAGGACAACGCCGAGGCCUCCACCCUGAUGGAGAUG |
| | | AAGGCCUACCACGCCAUCUCCCGGGCCCUGGAGAAGGAGGGCCUGAAGGACAAGAAGUCCCCCCUGAACCUGUCCUCCGAGCUGCAGGACG |
| | | AGAUCGGCACCGCCUUCUCCCUGUUCAAGACCGACGAGGACAUCACCGGCCGGCUGAAGGACCGGGUGCAGCCCGAGAUCCUGGAGGCCCU |
| | | GCUGAAGCACAUCUCCUUCGACAAGUUCGUGCAGAUCUCCCUGAAGGCCCUGCGGCGGAUCGUGCCCCUGAUGGAGCAGGGCAAGCGGUAC |
| | | GACGAGGCCUGCGCCGAGAUCUACGGCGACCACUACGGCAAGAAGAACACCGAGGAGAAGAUCUACCUGCCCCCCAUCCCCGCCGACGAGA |
| | | UCCGGAACCCCGUGGUGCUGCGGGCCCUGUCCCAGGCCCGGAAGGUGAUCAACGGCGUGGUGCGGCGGUACGGCUCCCCCGCCCGGAUCCA |
| | | CAUCGAGACCGCCCGGGAGGUGGGCAAGUCCUUCAAGGACCGGAAGGAGAUCGAGAAGCGGCAGGAGGAGAACCGGAAGGACCGGGAGAAG |
| | | GCCGCCGCCAAGUUCCGGGAGUACUUCCCCAACUUCGUGGGCGAGCCCAAGUCCAAGGACAUCCUGAAGCUGCGGCUGUACGAGCAGCAGC |
| | | ACGGCAAGUGCCUGUACUCCGGCAAGGAGAUCAACCUGGUGCGGCUGAACGAGAAGGGCUACGUGGAGAUCGACCACGCCCUGCCCUUCUC |
| | | CCGGACCUGGGACGACUCCUUCAACAACAAGGUGCUGGUGCUGGGCUCCGAGAACCAGAACAAGGGCAACCAGACCCCCUACGAGUACUUC |
| | | AACGGCAAGGACAACUCCCGGGAGUGGCAGGAGUUCAAGGCCCGGGUGGAGACCUCCCGGUUCCCCCGGUCCAAGAAGCAGCGGAUCCUGC |
| | | UGCAGAAGUUCGACGAGGACGGCUUCAAGGAGUGCAACCUGAACGACACCCGGUACGUGAACCGCUUCCUGUGCCAGUUCGUGGCCGACCA |
| | | CAUCCUGCUGACCGGCAAGGGCAAGCGGCGGGUGUUCGCCUCCAACGGCCAGAUCACCAACCUGCUGCGGGGCUUCUGGGGCCUGCGGAAG |
| | | GUGCGGGCCGAGAACGACCGGCACCACGCCCUGGACGCCGUGGUGGUGGCCUGCUCCACCGUGGCCAUGCAGCAGAAGAUCACCCGGUUCG |
| | | UGCGGUACAAGGAGAUGAACGCCUUCGACGGCAAGACCAUCGACAAGGAGACCGGCAAGGUGCUGCACCAGAAGACCCACUUCCCCCAGCC |
| | | CUGGGAGUUCUUCGCCCAGGAGGUGAUGAUCCGGGUGUUCGGCAAGCCCGACGGCAAGCCCGAGUUCGAGGAGGCCGACACCCCCGAGAAG |
| | | CUGCGGACCCUGCUGGCCGAGAAGCUGUCCUCCCGGCCCGAGGCCGUGCACGAGUACGUGACCCCCCUGUUCGUGUCCCGGGCCCCCAACC |
| | | GGAAGAUGUCCGGCGCCCACAAGGACACCCUGCGGUCCGCCAAGCGGUUCGUGAAGCACAACGAGAAGAUCUCCGUGAAGCGGGUGUGGCU |
| | | GACCGAGAUCAAGCUGGCCGACCUGGAGAACAUGGUGAACUACAAGAACGGCCGGGAGAUCGAGCUGUACGAGGCCCUGAAGGCCCGGCUG |
| | | GAGGCCUACGGCGGCAACGCCAAGCAGGCCUUCGACCCCAAGGACAACCCCUUCUACAAGAAGGGCGGCCAGCUGGUGAAGGCCGUGCGGG |
| | | UGGAGAAGACCCAGGAGUCCGGCGUGCUGCUGAACAAGAAGAACGCCUACACCAUCGCCGACAACGGCGACAUGGUGCGGGUGGACGUGUU |
| | | CUGCAAGGUGGACAAGAAGGGCAAGAACCAGUACUUCAUCGUGCCCAUCUACGCCUGGCAGGUGGCCGAGAACAUCCUGCCCGACAUCGAC |
| | | UGCAAGGGCUACCGGAUCGACGACUCCUACACCUUCUGCUUCUCCCUGCACAAGUACGACCUGAUCGCCUUCCAGAAGGACGAGAAGUCCA |
| | | AGGUGGAGUUCGCCUACUACAUCAACUGCGACUCCUCCAACGGCCGGUUCUACCUGGCCUGGCACGACAAGGGCUCCAAGGAGCAGCAGUU |
| | | CCGGAUCUCCACCCAGAACCUGGUGCUGAUCCAGAAGUACCAGGUGAACGAGCUGGGCAAGGAGAUCCGGCCCUGCCGGCUGAAGAAGCGG |
| | | CCCCCCGUGCGGUCCGGAAAGCGGACCGCCGACGGCUCCGGAGGAGGAAGCCCGAAGAAGAAGAGAAAGGUCAGCGAAAGCGCAACACCGG |
| | | AAAGCGUCAGCGGAUGGAGACUGUUCAAGAAGAUCAGCUAG |
| 80 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 11 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residue | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | and | GSPAIKKGILQTVKVVDELVKVMGRHKPENIVIEMARENQTTQKGQKNSRERMKRIEEGIKELGSQILKEHPVENTQLQNEKLYLYYLQNG |
| | HiBit | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | tag | LDKAGFIKRQLVETRQITKHVAQILDSRMNTKYDENDKLIREVKVITLKSKLVSDFRKDFQFYKVREINNYHHAHDAYLNAVVGTALIKKY |
| | (mRNA | PKLESEFVYGDYKVYDVRKMIAKSEQEIGKATAKYFFYSNIMNFFKTEITLANGEIRKRPLIETNGETGEIVWDKGRDFATVRKVLSMPQV |
| | U)- | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | amino | AKGYKEVKKDLIIKLPKYSLFELENGRKRMLASAGELQKGNELALPSKYVNFLYLASHYEKLKGSPEDNEQKQLFVEQHKHYLDEIIEQIS |
| | acid | EFSKRVILADANLDKVLSAYNKHRDKPIREQAENIIHLFTLTNLGAPAAFKYFDTTIDKKRYTSTKEVLDATLIHQSITGLYETRIDLSQL |
| | sequence | GGDGAPESATGSSSGAAFKPNPINYILGLDIGIASVGWAMVEIDEEENPIRLIDLGVRVFERAEVPKTGDSLAMARRLARSVRRLTRRRAH |
| | | RLLRARRLLKREGVLQAADFDENGLIKSLPNTPWQLRAAALDRKLTPLEWSAVLLHLIKHRGYLSQRKNEGETADKELGALLKGVANNAHA |
| | | LQTGDFRTPAELALNKFEKESGHIRNQRGDYSHTFSRKDLQAELILLFEKQKEFGNPHVSGGLKEGIETLLMTQRPALSGDAVQKMLGHCT |
| | | FEPAEPKAAKNTYTAERFIWLTKLNNLRILEQGSERPLTDTERATLMDEPYRKSKLTYAQARKLLGLEDTAFFKGLRYGKDNAEASTLMEM |
| | | KAYHAISRALEKEGLKDKKSPLNLSSELQDEIGTAFSLFKTDEDITGRLKDRVQPEILEALLKHISFDKFVQISLKALRRIVPLMEQGKRY |
| | | DEACAEIYGDHYGKKNTEEKIYLPPIPADEIRNPVVLRALSQARKVINGVVRRYGSPARIHIETAREVGKSFKDRKEIEKRQEENRKDREK |
| | | AAAKFREYFPNFVGEPKSKDILKLRLYEQQHGKCLYSGKEINLVRLNEKGYVEIDHALPFSRTWDDSFNNKVLVLGSENQNKGNQTPYEYF |
| | | NGKDNSREWQEFKARVETSRFPRSKKQRILLQKFDEDGFKECNLNDTRYVNRFLCQFVADHILLTGKGKRRVFASNGQITNLLRGFWGLRK |
| | | VRAENDRHHALDAVVVACSTVAMQQKITRFVRYKEMNAFDGKTIDKETGKVLHQKTHFPQPWEFFAQEVMIRVFGKPDGKPEFEEADTPEK |
| | | LRTLLAEKLSSRPEAVHEYVTPLFVSRAPNRKMSGAHKDTLRSAKRFVKHNEKISVKRVWLTEIKLADLENMVNYKNGREIELYEALKARL |
| | | EAYGGNAKQAFDPKDNPFYKKGGQLVKAVRVEKTQESGVLLNKKNAYTIADNGDMVRVDVFCKVDKKGKNQYFIVPIYAWQVAENILPDID |
| | | CKGYRIDDSYTFCFSLHKYDLIAFQKDEKSKVEFAYYINCDSSNGRFYLAWHDKGSKEQQFRISTQNLVLIQKYQVNELGKEIRPCRLKKR |
| | | PPVRSGKRTADGSGGGSPKKKRKVSESATPESVSGWRLFKKIS* |
| 81 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 11 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | (mRNA | CCUGUCCCUGGGCCUGACCCCCAACUUCAAGUCCAACUUCGACCUGGCCGAGGACGCCAAGCUGCAGCUGUCCAAGGACACCUACGACGAC |
| | U)- | GACCUGGACAACCUGCUGGCCCAGAUCGGCGACCAGUACGCCGACCUGUUCCUGGCCGCCAAGAACCUGUCCGACGCCAUCCUGCUGUCCG |
| | mRNA | ACAUCCUGCGGGUGAACACCGAGAUCACCAAGGCCCCCCUGUCCGCCUCCAUGAUCAAGCGGUACGACGAGCACCACCAGGACCUGACCCU |
| | sequence | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | | GGGAGGACCUGCUGCGGAAGCAGCGGACCUUCGACAACGGCUCCAUCCCCCACCAGAUCCACCUGGGCGAGCUGCACGCCAUCCUGCGGCG |
| | | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGGUGAAGUACGUGACCGAGGGCAUGCGGAAGCCCGCCUUCCUGUCCGGCGAGCAGAAGAAG |
| | | GCCAUCGUGGACCUGCUGUUCAAGACCAACCGGAAGGUGACCGUGAAGCAGCUGAAGGAGGACUACUUCAAGAAGAUCGAGUGCUUCGACU |
| | | CCGUGGAGAUCUCCGGCGUGGAGGACCGGUUCAACGCCUCCCUGGGCACCUACCACGACCUGCUGAAGAUCAUCAAGGACAAGGACUUCCU |
| | | GGACAACGAGGAGAACGAGGACAUCCUGGAGGACAUCGUGCUGACCCUGACCCUGUUCGAGGACCGGGAGAUGAUCGAGGAGCGGCUGAAG |
| | | ACCUACGCCCACCUGUUCGACGACAAGGUGAUGAAGCAGCUGAAGCGGCGGCGGUACACCGGCUGGGGCCGGCUGUCCCGGAAGCUGAUCA |
| | | ACGGCAUCCGGGACAAGCAGUCCGGCAAGACCAUCCUGGACUUCCUGAAGUCCGACGGCUUCGCCAACCGGAACUUCAUGCAGCUGAUCCA |
| | | CGACGACUCCCUGACCUUCAAGGAGGACAUCCAGAAGGCCCAGGUGUCCGGCCAGGGCGACUCCCUGCACGAGCACAUCGCCAACCUGGCC |
| | | GGCUCCCCCGCCAUCAAGAAGGGCAUCCUGCAGACCGUGAAGGUGGUGGACGAGCUGGUGAAGGUGAUGGGCCGGCACAAGCCCGAGAACA |
| | | UCGUGAUCGAGAUGGCCCGGGAGAACCAGACCACCCAGAAGGGCCAGAAGAACUCCCGGGAGCGGAUGAAGCGGAUCGAGGAGGGCAUCAA |
| | | GGAGCUGGGCUCCCAGAUCCUGAAGGAGCACCCCGUGGAGAACACCCAGCUGCAGAACGAGAAGCUGUACCUGUACUACCUGCAGAACGGC |
| | | CGGGACAUGUACGUGGACCAGGAGCUGGACAUCAACCGGCUGUCCGACUACGACGUGGACCACAUCGUGCCCCAGUCCUUCCUGAAGGACG |
| | | ACUCCAUCGACAACAAGGUGCUGACCCGGUCCGACAAGAACCGGGGCAAGUCCGACAACGUGCCCUCCGAGGAGGUGGUGAAGAAGAUGAA |
| | | GAACUACUGGCGGCAGCUGCUGAACGCCAAGCUGAUCACCCAGCGGAAGUUCGACAACCUGACCAAGGCCGAGCGGGGCGGCCUGUCCGAG |
| | | CUGGACAAGGCCGGCUUCAUCAAGCGGCAGCUGGUGGAGACCCGGCAGAUCACCAAGCACGUGGCCCAGAUCCUGGACUCCCGGAUGAACA |
| | | CCAAGUACGACGAGAACGACAAGCUGAUCCGGGAGGUGAAGGUGAUCACCCUGAAGUCCAAGCUGGUGUCCGACUUCCGGAAGGACUUCCA |
| | | GUUCUACAAGGUGCGGGAGAUCAACAACUACCACCACGCCCACGACGCCUACCUGAACGCCGUGGUGGGCACCGCCCUGAUCAAGAAGUAC |
| | | CCCAAGCUGGAGUCCGAGUUCGUGUACGGCGACUACAAGGUGUACGACGUGCGGAAGAUGAUCGCCAAGUCCGAGCAGGAGAUCGGCAAGG |
| | | CCACCGCCAAGUACUUCUUCUACUCCAACAUCAUGAACUUCUUCAAGACCGAGAUCACCCUGGCCAACGGCGAGAUCCGGAAGCGGCCCCU |
| | | GAUCGAGACCAACGGCGAGACCGGCGAGAUCGUGUGGGACAAGGGCCGGGACUUCGCCACCGUGCGGAAGGUGCUGUCCAUGCCCCAGGUG |
| | | AACAUCGUGAAGAAGACCGAGGUGCAGACCGGCGGCUUCUCCAAGGAGUCCAUCCUGCCCAAGCGGAACUCCGACAAGCUGAUCGCCCGGA |
| | | AGAAGGACUGGGACCCCAAGAAGUACGGCGGCUUCGACUCCCCCACCGUGGCCUACUCCGUGCUGGUGGUGGCCAAGGUGGAGAAGGGCAA |
| | | GUCCAAGAAGCUGAAGUCCGUGAAGGAGCUGCUGGGCAUCACCAUCAUGGAGCGGUCCUCCUUCGAGAAGAACCCCAUCGACUUCCUGGAG |
| | | GCCAAGGGCUACAAGGAGGUGAAGAAGGACCUGAUCAUCAAGCUGCCCAAGUACUCCCUGUUCGAGCUGGAGAACGGCCGGAAGCGGAUGC |
| | | UGGCCUCCGCCGGCGAGCUGCAGAAGGGCAACGAGCUGGCCCUGCCCUCCAAGUACGUGAACUUCCUGUACCUGGCCUCCCACUACGAGAA |
| | | GCUGAAGGGCUCCCCCGAGGACAACGAGCAGAAGCAGCUGUUCGUGGAGCAGCACAAGCACUACCUGGACGAGAUCAUCGAGCAGAUCUCC |
| | | GAGUUCUCCAAGCGGGUGAUCCUGGCCGACGCCAACCUGGACAAGGUGCUGUCCGCCUACAACAAGCACCGGGACAAGCCCAUCCGGGAGC |
| | | AGGCCGAGAACAUCAUCCACCUGUUCACCCUGACCAACCUGGGCGCCCCCGCCGCCUUCAAGUACUUCGACACCACCAUCGACAAGAAGCG |
| | | GUACACCUCCACCAAGGAGGUGCUGGACGCCACCCUGAUCCACCAGUCCAUCACCGGCCUGUACGAGACCCGGAUCGACCUGUCCCAGCUG |
| | | GGCGGCGACGGCGCCCCCGAGUCCGCCACCGGCUCCUCCUCCGGUGCCGCCUUCAAGCCCAACCCCAUCAACUACAUCCUGGGCCUGGACA |
| | | UCGGCAUCGCCUCCGUGGGCUGGGCCAUGGUGGAGAUCGACGAGGAGGAGAACCCCAUCCGGCUGAUCGACCUGGGCGUGCGGGUGUUCGA |
| | | GCGGGCCGAGGUGCCCAAGACCGGCGACUCCCUGGCCAUGGCCCGGCGGCUGGCCCGGUCCGUGCGGCGGCUGACCCGGCGGCGGGCCCAC |
| | | CGGCUGCUGCGGGCCCGGCGGCUGCUGAAGCGGGAGGGCGUGCUGCAGGCCGCCGACUUCGACGAGAACGGCCUGAUCAAGUCCCUGCCCA |
| | | ACACCCCCUGGCAGCUGCGGGCCGCCGCCCUGGACCGGAAGCUGACCCCCCUGGAGUGGUCCGCCGUGCUGCUGCACCUGAUCAAGCACCG |
| | | GGGCUACCUGUCCCAGCGGAAGAACGAGGGCGAGACCGCCGACAAGGAGCUGGGCGCCCUGCUGAAGGGCGUGGCCAACAACGCCCACGCC |
| | | CUGCAGACCGGCGACUUCCGGACCCCCGCCGAGCUGGCCCUGAACAAGUUCGAGAAGGAGUCCGGCCACAUCCGGAACCAGCGGGGCGACU |
| | | ACUCCCACACCUUCUCCCGGAAGGACCUGCAGGCCGAGCUGAUCCUGCUGUUCGAGAAGCAGAAGGAGUUCGGCAACCCCCACGUGUCCGG |
| | | CGGCCUGAAGGAGGGCAUCGAGACCCUGCUGAUGACCCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCAUCUGGCUGACCAAGCUGAACAACCUGCGGAUCCUGG |
| | | AGCAGGGCUCCGAGCGGCCCCUGACCGACACCGAGCGGGCCACCCUGAUGGACGAGCCCUACCGGAAGUCCAAGCUGACCUACGCCCAGGC |
| | | CCGGAAGCUGCUGGGCCUGGAGGACACCGCCUUCUUCAAGGGCCUGCGGUACGGCAAGGACAACGCCGAGGCCUCCACCCUGAUGGAGAUG |
| | | AAGGCCUACCACGCCAUCUCCCGGGCCCUGGAGAAGGAGGGCCUGAAGGACAAGAAGUCCCCCCUGAACCUGUCCUCCGAGCUGCAGGACG |
| | | AGAUCGGCACCGCCUUCUCCCUGUUCAAGACCGACGAGGACAUCACCGGCCGGCUGAAGGACCGGGUGCAGCCCGAGAUCCUGGAGGCCCU |
| | | GCUGAAGCACAUCUCCUUCGACAAGUUCGUGCAGAUCUCCCUGAAGGCCCUGCGGCGGAUCGUGCCCCUGAUGGAGCAGGGCAAGCGGUAC |
| | | GACGAGGCCUGCGCCGAGAUCUACGGCGACCACUACGGCAAGAAGAACACCGAGGAGAAGAUCUACCUGCCCCCCAUCCCCGCCGACGAGA |
| | | UCCGGAACCCCGUGGUGCUGCGGGCCCUGUCCCAGGCCCGGAAGGUGAUCAACGGCGUGGUGCGGCGGUACGGCUCCCCCGCCCGGAUCCA |
| | | CAUCGAGACCGCCCGGGAGGUGGGCAAGUCCUUCAAGGACCGGAAGGAGAUCGAGAAGCGGCAGGAGGAGAACCGGAAGGACCGGGAGAAG |
| | | GCCGCCGCCAAGUUCCGGGAGUACUUCCCCAACUUCGUGGGCGAGCCCAAGUCCAAGGACAUCCUGAAGCUGCGGCUGUACGAGCAGCAGC |
| | | ACGGCAAGUGCCUGUACUCCGGCAAGGAGAUCAACCUGGUGCGGCUGAACGAGAAGGGCUACGUGGAGAUCGACCACGCCCUGCCCUUCUC |
| | | CCGGACCUGGGACGACUCCUUCAACAACAAGGUGCUGGUGCUGGGCUCCGAGAACCAGAACAAGGGCAACCAGACCCCCUACGAGUACUUC |
| | | AACGGCAAGGACAACUCCCGGGAGUGGCAGGAGUUCAAGGCCCGGGUGGAGACCUCCCGGUUCCCCCGGUCCAAGAAGCAGCGGAUCCUGC |
| | | UGCAGAAGUUCGACGAGGACGGCUUCAAGGAGUGCAACCUGAACGACACCCGGUACGUGAACCGCUUCCUGUGCCAGUUCGUGGCCGACCA |
| | | CAUCCUGCUGACCGGCAAGGGCAAGCGGCGGGUGUUCGCCUCCAACGGCCAGAUCACCAACCUGCUGCGGGGCUUCUGGGGCCUGCGGAAG |
| | | GUGCGGGCCGAGAACGACCGGCACCACGCCCUGGACGCCGUGGUGGUGGCCUGCUCCACCGUGGCCAUGCAGCAGAAGAUCACCCGGUUCG |
| | | UGCGGUACAAGGAGAUGAACGCCUUCGACGGCAAGACCAUCGACAAGGAGACCGGCAAGGUGCUGCACCAGAAGACCCACUUCCCCCAGCC |
| | | CUGGGAGUUCUUCGCCCAGGAGGUGAUGAUCCGGGUGUUCGGCAAGCCCGACGGCAAGCCCGAGUUCGAGGAGGCCGACACCCCCGAGAAG |
| | | CUGCGGACCCUGCUGGCCGAGAAGCUGUCCUCCCGGCCCGAGGCCGUGCACGAGUACGUGACCCCCCUGUUCGUGUCCCGGGCCCCCAACC |
| | | GGAAGAUGUCCGGCGCCCACAAGGACACCCUGCGGUCCGCCAAGCGGUUCGUGAAGCACAACGAGAAGAUCUCCGUGAAGCGGGUGUGGCU |
| | | GACCGAGAUCAAGCUGGCCGACCUGGAGAACAUGGUGAACUACAAGAACGGCCGGGAGAUCGAGCUGUACGAGGCCCUGAAGGCCCGGCUG |
| | | GAGGCCUACGGCGGCAACGCCAAGCAGGCCUUCGACCCCAAGGACAACCCCUUCUACAAGAAGGGCGGCCAGCUGGUGAAGGCCGUGCGGG |
| | | UGGAGAAGACCCAGGAGUCCGGCGUGCUGCUGAACAAGAAGAACGCCUACACCAUCGCCGACAACGGCGACAUGGUGCGGGUGGACGUGUU |
| | | CUGCAAGGUGGACAAGAAGGGCAAGAACCAGUACUUCAUCGUGCCCAUCUACGCCUGGCAGGUGGCCGAGAACAUCCUGCCCGACAUCGAC |
| | | UGCAAGGGCUACCGGAUCGACGACUCCUACACCUUCUGCUUCUCCCUGCACAAGUACGACCUGAUCGCCUUCCAGAAGGACGAGAAGUCCA |
| | | AGGUGGAGUUCGCCUACUACAUCAACUGCGACUCCUCCAACGGCCGGUUCUACCUGGCCUGGCACGACAAGGGCUCCAAGGAGCAGCAGUU |
| | | CCGGAUCUCCACCCAGAACCUGGUGCUGAUCCAGAAGUACCAGGUGAACGAGCUGGGCAAGGAGAUCCGGCCCUGCCGGCUGAAGAAGCGG |
| | | CCCCCCGUGCGGUCCGGAAAGCGGACCGCCGACGGCUCCGGAGGAGGAAGCCCGAAGAAGAAGAGAAAGGUCUAG |
| 82 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9- | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | 11 | KLFIQLVQTYNQLFEENPINASGVDAKAILSARLSKSRRLENLIAQLPGEKKNGLFGNLIALSLGLTPNFKSNFDLAEDAKLQLSKDTYDD |
| | fusion | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | residual | AIVDLLFKTNRKVTVKQLKEDYFKKIECFDSVEISGVEDRFNASLGTYHDLLKIIKDKDFLDNEENEDILEDIVLTLTLFEDREMIEERLK |
| | linker | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | (mRNA | GSPAIKKGILQTVKVVDELVKVMGRHKPENIVIEMARENQTTQKGQKNSRERMKRIEEGIKELGSQILKEHPVENTQLQNEKLYLYYLQNG |
| | U)- | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | amino | LDKAGFIKRQLVETRQITKHVAQILDSRMNTKYDENDKLIREVKVITLKSKLVSDFRKDFQFYKVREINNYHHAHDAYLNAVVGTALIKKY |
| | acid | PKLESEFVYGDYKVYDVRKMIAKSEQEIGKATAKYFFYSNIMNFFKTEITLANGEIRKRPLIETNGETGEIVWDKGRDFATVRKVLSMPQV |
| | sequence | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATGSSSGAAFKPNPINYILGLDIGIASVGWAMVEIDEEENPIRLIDLGVRVFERAEVPKTGDSLAMARRLARSVRRLTRRRAH |
| | | RLLRARRLLKREGVLQAADFDENGLIKSLPNTPWQLRAAALDRKLTPLEWSAVLLHLIKHRGYLSQRKNEGETADKELGALLKGVANNAHA |
| | | LQTGDFRTPAELALNKFEKESGHIRNQRGDYSHTFSRKDLQAELILLFEKQKEFGNPHVSGGLKEGIETLLMTQRPALSGDAVQKMLGHCT |
| | | FEPAEPKAAKNTYTAERFIWLTKLNNLRILEQGSERPLTDTERATLMDEPYRKSKLTYAQARKLLGLEDTAFFKGLRYGKDNAEASTLMEM |
| | | KAYHAISRALEKEGLKDKKSPLNLSSELQDEIGTAFSLFKTDEDITGRLKDRVQPEILEALLKHISFDKFVQISLKALRRIVPLMEQGKRY |
| | | DEACAEIYGDHYGKKNTEEKIYLPPIPADEIRNPVVLRALSQARKVINGVVRRYGSPARIHIETAREVGKSFKDRKEIEKRQEENRKDREK |
| | | AAAKFREYFPNFVGEPKSKDILKLRLYEQQHGKCLYSGKEINLVRLNEKGYVEIDHALPFSRTWDDSFNNKVLVLGSENQNKGNQTPYEYF |
| | | NGKDNSREWQEFKARVETSRFPRSKKQRILLQKFDEDGFKECNLNDTRYVNRFLCQFVADHILLTGKGKRRVFASNGQITNLLRGFWGLRK |
| | | VRAENDRHHALDAVVVACSTVAMQQKITRFVRYKEMNAFDGKTIDKETGKVLHQKTHFPQPWEFFAQEVMIRVFGKPDGKPEFEEADTPEK |
| | | LRTLLAEKLSSRPEAVHEYVTPLFVSRAPNRKMSGAHKDTLRSAKRFVKHNEKISVKRVWLTEIKLADLENMVNYKNGREIELYEALKARL |
| | | EAYGGNAKQAFDPKDNPFYKKGGQLVKAVRVEKTQESGVLLNKKNAYTIADNGDMVRVDVFCKVDKKGKNQYFIVPIYAWQVAENILPDID |
| | | CKGYRIDDSYTFCFSLHKYDLIAFQKDEKSKVEFAYYINCDSSNGRFYLAWHDKGSKEQQFRISTQNLVLIQKYQVNELGKEIRPCRLKKR |
| | | PPVRSGKRTADGSGGGSPKKKRKV* |
| 83 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 81 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGGGCGGCGGCGGCUCCACCGAGAUCACCAAGGCCCCCCUGUCCGCCUCCAUGAUCAAGCGGUACGACGAGCA |
| | linker | CCACCAGGACCUGACCCUGCUGAAGGCCCUGGUGCGGCAGCAGCUGCCCGAGAAGUACAAGGAGAUCUUCUUCGACCAGUCCAAGAACGGC |
| | and | UACGCCGGCUACAUCGACGGCGGCGCCUCCCAGGAGGAGUUCUACAAGUUCAUCAAGCCCAUCCUGGAGAAGAUGGACGGCACCGAGGAGC |
| | HiBit | UGCUGGUGAAGCUGAACCGGGAGGACCUGCUGCGGAAGCAGCGGACCUUCGACAACGGCUCCAUCCCCCACCAGAUCCACCUGGGCGAGCU |
| | tag- | GCACGCCAUCCUGCGGCGGCAGGAGGACUUCUACCCCUUCCUGAAGGACAACCGGGAGAAGAUCGAGAAGAUCCUGACCUUCCGGAUCCCC |
| | SpyCas9 | UACUACGUGGGCCCCCUGGCCCGGGGCAACUCCCGGUUCGCCUGGAUGACCCGGAAGUCCGAGGAGACCAUCACCCCCUGGAACUUCGAGG |
| | without | AGGUGGUGGACAAGGGCGCCUCCGCCCAGUCCUUCAUCGAGCGGAUGACCAACUUCGACAAGAACCUGCCCAACGAGAAGGUGCUGCCCAA |
| | Rec2 | GCACUCCCUGCUGUACGAGUACUUCACCGUGUACAACGAGCUGACCAAGGUGAAGUACGUGACCGAGGGCAUGCGGAAGCCCGCCUUCCUG |
| | domain | UCCGGCGAGCAGAAGAAGGCCAUCGUGGACCUGCUGUUCAAGACCAACCGGAAGGUGACCGUGAAGCAGCUGAAGGAGGACUACUUCAAGA |
| | (mRNA | AGAUCGAGUGCUUCGACUCCGUGGAGAUCUCCGGCGUGGAGGACCGGUUCAACGCCUCCCUGGGCACCUACCACGACCUGCUGAAGAUCAU |
| | V)- | CAAGGACAAGGACUUCCUGGACAACGAGGAGAACGAGGACAUCCUGGAGGACAUCGUGCUGACCCUGACCCUGUUCGAGGACCGGGAGAUG |
| | mRNA | AUCGAGGAGCGGCUGAAGACCUACGCCCACCUGUUCGACGACAAGGUGAUGAAGCAGCUGAAGCGGCGGCGGUACACCGGCUGGGGCCGGC |
| | sequence | UGUCCCGGAAGCUGAUCAACGGCAUCCGGGACAAGCAGUCCGGCAAGACCAUCCUGGACUUCCUGAAGUCCGACGGCUUCGCCAACCGGAA |
| | | CUUCAUGCAGCUGAUCCACGACGACUCCCUGACCUUCAAGGAGGACAUCCAGAAGGCCCAGGUGUCCGGCCAGGGCGACUCCCUGCACGAG |
| | | CACAUCGCCAACCUGGCCGGCUCCCCCGCCAUCAAGAAGGGCAUCCUGCAGACCGUGAAGGUGGUGGACGAGCUGGUGAAGGUGAUGGGCC |
| | | GGCACAAGCCCGAGAACAUCGUGAUCGAGAUGGCCCGGGAGAACCAGACCACCCAGAAGGGCCAGAAGAACUCCCGGGAGCGGAUGAAGCG |
| | | GAUCGAGGAGGGCAUCAAGGAGCUGGGCUCCCAGAUCCUGAAGGAGCACCCCGUGGAGAACACCCAGCUGCAGAACGAGAAGCUGUACCUG |
| | | UACUACCUGCAGAACGGCCGGGACAUGUACGUGGACCAGGAGCUGGACAUCAACCGGCUGUCCGACUACGACGUGGACCACAUCGUGCCCC |
| | | AGUCCUUCCUGAAGGACGACUCCAUCGACAACAAGGUGCUGACCCGGUCCGACAAGAACCGGGGCAAGUCCGACAACGUGCCCUCCGAGGA |
| | | GGUGGUGAAGAAGAUGAAGAACUACUGGCGGCAGCUGCUGAACGCCAAGCUGAUCACCCAGCGGAAGUUCGACAACCUGACCAAGGCCGAG |
| | | CGGGGCGGCCUGUCCGAGCUGGACAAGGCCGGCUUCAUCAAGCGGCAGCUGGUGGAGACCCGGCAGAUCACCAAGCACGUGGCCCAGAUCC |
| | | UGGACUCCCGGAUGAACACCAAGUACGACGAGAACGACAAGCUGAUCCGGGAGGUGAAGGUGAUCACCCUGAAGUCCAAGCUGGUGUCCGA |
| | | CUUCCGGAAGGACUUCCAGUUCUACAAGGUGCGGGAGAUCAACAACUACCACCACGCCCACGACGCCUACCUGAACGCCGUGGUGGGCACC |
| | | GCCCUGAUCAAGAAGUACCCCAAGCUGGAGUCCGAGUUCGUGUACGGCGACUACAAGGUGUACGACGUGCGGAAGAUGAUCGCCAAGUCCG |
| | | AGCAGGAGAUCGGCAAGGCCACCGCCAAGUACUUCUUCUACUCCAACAUCAUGAACUUCUUCAAGACCGAGAUCACCCUGGCCAACGGCGA |
| | | GAUCCGGAAGCGGCCCCUGAUCGAGACCAACGGCGAGACCGGCGAGAUCGUGUGGGACAAGGGCCGGGACUUCGCCACCGUGCGGAAGGUG |
| | | CUGUCCAUGCCCCAGGUGAACAUCGUGAAGAAGACCGAGGUGCAGACCGGCGGCUUCUCCAAGGAGUCCAUCCUGCCCAAGCGGAACUCCG |
| | | ACAAGCUGAUCGCCCGGAAGAAGGACUGGGACCCCAAGAAGUACGGCGGCUUCGACUCCCCCACCGUGGCCUACUCCGUGCUGGUGGUGGC |
| | | CAAGGUGGAGAAGGGCAAGUCCAAGAAGCUGAAGUCCGUGAAGGAGCUGCUGGGCAUCACCAUCAUGGAGCGGUCCUCCUUCGAGAAGAAC |
| | | CCCAUCGACUUCCUGGAGGCCAAGGGCUACAAGGAGGUGAAGAAGGACCUGAUCAUCAAGCUGCCCAAGUACUCCCUGUUCGAGCUGGAGA |
| | | ACGGCCGGAAGCGGAUGCUGGCCUCCGCCGGCGAGCUGCAGAAGGGCAACGAGCUGGCCCUGCCCUCCAAGUACGUGAACUUCCUGUACCU |
| | | GGCCUCCCACUACGAGAAGCUGAAGGGCUCCCCCGAGGACAACGAGCAGAAGCAGCUGUUCGUGGAGCAGCACAAGCACUACCUGGACGAG |
| | | AUCAUCGAGCAGAUCUCCGAGUUCUCCAAGCGGGUGAUCCUGGCCGACGCCAACCUGGACAAGGUGCUGUCCGCCUACAACAAGCACCGGG |
| | | ACAAGCCCAUCCGGGAGCAGGCCGAGAACAUCAUCCACCUGUUCACCCUGACCAACCUGGGCGCCCCCGCCGCCUUCAAGUACUUCGACAC |
| | | CACCAUCGACAAGAAGCGGUACACCUCCACCAAGGAGGUGCUGGACGCCACCCUGAUCCACCAGUCCAUCACCGGCCUGUACGAGACCCGG |
| | | AUCGACCUGUCCCAGCUGGGCGGCGACGGCGCCCCCGAGUCCGCCACCGAGUCCGGCGGCACCUCCACCGAGUCCGAGGGCUCCGCCGGCA |
| | | CCUCCACCGAGUCCGAGGGCUCCGCCGGCUCCGCCGGCUCCACCUCCACCGAGGAGGGCACCUCCACCGAGUCCGAGGGCUCCGCCGGCAC |
| | | CUCCACCGAGUCCGAGGGCUCCGCCGGCACCUCCGAGUCCGCCACCGAGUCCGGCGGCACCUCCACCGAGUCCGAGGGCUCCUCCUCCGGU |
| | | GCCGCCUUCAAGCCCAACCCCAUCAACUACAUCCUGGGCCUGGACAUCGGCAUCGCCUCCGUGGGCUGGGCCAUGGUGGAGAUCGACGAGG |
| | | AGGAGAACCCCAUCCGGCUGAUCGACCUGGGCGUGCGGGUGUUCGAGCGGGCCGAGGUGCCCAAGACCGGCGACUCCCUGGCCAUGGCCCG |
| | | GCGGCUGGCCCGGUCCGUGCGGCGGCUGACCCGGCGGCGGGCCCACCGGCUGCUGCGGGCCCGGCGGCUGCUGAAGCGGGAGGGCGUGCUG |
| | | CAGGCCGCCGACUUCGACGAGAACGGCCUGAUCAAGUCCCUGCCCAACACCCCCUGGCAGCUGCGGGCCGCCGCCCUGGACCGGAAGCUGA |
| | | CCCCCCUGGAGUGGUCCGCCGUGCUGCUGCACCUGAUCAAGCACCGGGGCUACCUGUCCCAGCGGAAGAACGAGGGCGAGACCGCCGACAA |
| | | GGAGCUGGGCGCCCUGCUGAAGGGCGUGGCCAACAACGCCCACGCCCUGCAGACCGGCGACUUCCGGACCCCCGCCGAGCUGGCCCUGAAC |
| | | AAGUUCGAGAAGGAGUCCGGCCACAUCCGGAACCAGCGGGGCGACUACUCCCACACCUUCUCCCGGAAGGACCUGCAGGCCGAGCUGAUCC |
| | | UGCUGUUCGAGAAGCAGAAGGAGUUCGGCAACCCCCACGUGUCCGGCGGCCUGAAGGAGGGCAUCGAGACCCUGCUGAUGACCCAGCGGCC |
| | | CGCCCUGUCCGGCGACGCCGUGCAGAAGAUGCUGGGCCACUGCACCUUCGAGCCCGCCGAGCCCAAGGCCGCCAAGAACACCUACACCGCC |
| | | GAGCGGUUCAUCUGGCUGACCAAGCUGAACAACCUGCGGAUCCUGGAGCAGGGCUCCGAGCGGCCCCUGACCGACACCGAGCGGGCCACCC |
| | | UGAUGGACGAGCCCUACCGGAAGUCCAAGCUGACCUACGCCCAGGCCCGGAAGCUGCUGGGCCUGGAGGACACCGCCUUCUUCAAGGGCCU |
| | | GCGGUACGGCAAGGACAACGCCGAGGCCUCCACCCUGAUGGAGAUGAAGGCCUACCACGCCAUCUCCCGGGCCCUGGAGAAGGAGGGCCUG |
| | | AAGGACAAGAAGUCCCCCCUGAACCUGUCCUCCGAGCUGCAGGACGAGAUCGGCACCGCCUUCUCCCUGUUCAAGACCGACGAGGACAUCA |
| | | CCGGCCGGCUGAAGGACCGGGUGCAGCCCGAGAUCCUGGAGGCCCUGCUGAAGCACAUCUCCUUCGACAAGUUCGUGCAGAUCUCCCUGAA |
| | | GGCCCUGCGGCGGAUCGUGCCCCUGAUGGAGCAGGGCAAGCGGUACGACGAGGCCUGCGCCGAGAUCUACGGCGACCACUACGGCAAGAAG |
| | | AACACCGAGGAGAAGAUCUACCUGCCCCCCAUCCCCGCCGACGAGAUCCGGAACCCCGUGGUGCUGCGGGCCCUGUCCCAGGCCCGGAAGG |
| | | UGAUCAACGGCGUGGUGCGGCGGUACGGCUCCCCCGCCCGGAUCCACAUCGAGACCGCCCGGGAGGUGGGCAAGUCCUUCAAGGACCGGAA |
| | | GGAGAUCGAGAAGCGGCAGGAGGAGAACCGGAAGGACCGGGAGAAGGCCGCCGCCAAGUUCCGGGAGUACUUCCCCAACUUCGUGGGCGAG |
| | | CCCAAGUCCAAGGACAUCCUGAAGCUGCGGCUGUACGAGCAGCAGCACGGCAAGUGCCUGUACUCCGGCAAGGAGAUCAACCUGGUGCGGC |
| | | UGAACGAGAAGGGCUACGUGGAGAUCGACCACGCCCUGCCCUUCUCCCGGACCUGGGACGACUCCUUCAACAACAAGGUGCUGGUGCUGGG |
| | | CUCCGAGAACCAGAACAAGGGCAACCAGACCCCCUACGAGUACUUCAACGGCAAGGACAACUCCCGGGAGUGGCAGGAGUUCAAGGCCCGG |
| | | GUGGAGACCUCCCGGUUCCCCCGGUCCAAGAAGCAGCGGAUCCUGCUGCAGAAGUUCGACGAGGACGGCUUCAAGGAGUGCAACCUGAACG |
| | | ACACCCGGUACGUGAACCGCUUCCUGUGCCAGUUCGUGGCCGACCACAUCCUGCUGACCGGCAAGGGCAAGCGGCGGGUGUUCGCCUCCAA |
| | | CGGCCAGAUCACCAACCUGCUGCGGGGCUUCUGGGGCCUGCGGAAGGUGCGGGCCGAGAACGACCGGCACCACGCCCUGGACGCCGUGGUG |
| | | GUGGCCUGCUCCACCGUGGCCAUGCAGCAGAAGAUCACCCGGUUCGUGCGGUACAAGGAGAUGAACGCCUUCGACGGCAAGACCAUCGACA |
| | | AGGAGACCGGCAAGGUGCUGCACCAGAAGACCCACUUCCCCCAGCCCUGGGAGUUCUUCGCCCAGGAGGUGAUGAUCCGGGUGUUCGGCAA |
| | | GCCCGACGGCAAGCCCGAGUUCGAGGAGGCCGACACCCCCGAGAAGCUGCGGACCCUGCUGGCCGAGAAGCUGUCCUCCCGGCCCGAGGCC |
| | | GUGCACGAGUACGUGACCCCCCUGUUCGUGUCCCGGGCCCCCAACCGGAAGAUGUCCGGCGCCCACAAGGACACCCUGCGGUCCGCCAAGC |
| | | GGUUCGUGAAGCACAACGAGAAGAUCUCCGUGAAGCGGGUGUGGCUGACCGAGAUCAAGCUGGCCGACCUGGAGAACAUGGUGAACUACAA |
| | | GAACGGCCGGGAGAUCGAGCUGUACGAGGCCCUGAAGGCCCGGCUGGAGGCCUACGGCGGCAACGCCAAGCAGGCCUUCGACCCCAAGGAC |
| | | AACCCCUUCUACAAGAAGGGCGGCCAGCUGGUGAAGGCCGUGCGGGUGGAGAAGACCCAGGAGUCCGGCGUGCUGCUGAACAAGAAGAACG |
| | | CCUACACCAUCGCCGACAACGGCGACAUGGUGCGGGUGGACGUGUUCUGCAAGGUGGACAAGAAGGGCAAGAACCAGUACUUCAUCGUGCC |
| | | CAUCUACGCCUGGCAGGUGGCCGAGAACAUCCUGCCCGACAUCGACUGCAAGGGCUACCGGAUCGACGACUCCUACACCUUCUGCUUCUCC |
| | | CUGCACAAGUACGACCUGAUCGCCUUCCAGAAGGACGAGAAGUCCAAGGUGGAGUUCGCCUACUACAUCAACUGCGACUCCUCCAACGGCC |
| | | GGUUCUACCUGGCCUGGCACGACAAGGGCUCCAAGGAGCAGCAGUUCCGGAUCUCCACCCAGAACCUGGUGCUGAUCCAGAAGUACCAGGU |
| | | GAACGAGCUGGGCAAGGAGAUCCGGCCCUGCCGGCUGAAGAAGCGGCCCCCCGUGCGGUCCGGAAAGCGGACCGCCGACGGCUCCGGAGGA |
| | | GGAAGCCCGAAGAAGAAGAGAAAGGUCAGCGAAAGCGCAACACCGGAAAGCGUCAGCGGAUGGAGACUGUUCAAGAAGAUCAGCUAGCUAG |
| | | CACCAGCCUCAAGAACACCCGAAUGGAGUCUCUAAGCUACAUAAUACCAACUUACACUUUACAAAAUGUUGUCCCCCAAAAUGUAGCCAUU |
| | | CGUAUCUGCUCCUAAUAAAAAGAAAGUUUCUUCACAUUCUCUCGAGAAAAAAAAAAAAUGGAAAAAAAAAAAACGGAAAAAAAAAAAAGGU |
| | | AAAAAAAAAAAAUAUAAAAAAAAAAAACAUAAAAAAAAAAAACGAAAAAAAAAAAACGUAAAAAAAAAAAACUCAAAAAAAAAAAAGAUAA |
| | | AAAAAAAAAACCUAAAAAAAAAAAAUGUAAAAAAAAAAAAGGGAAAAAAAAAAAACGCAAAAAAAAAAAACACAAAAAAAAAAAAUGCAAA |
| | | AAAAAAAAAUCGAAAAAAAAAAAAUCUAAAAAAAAAAAACGAAAAAAAAAAAACCCAAAAAAAAAAAAGACAAAAAAAAAAAAUAGAAAAA |
| | | AAAAAAAGUUAAAAAAAAAAAACUGAAAAAAAAAAAAUUUAAAAAAAAAAAAUCUAG |
| 84 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 81 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGGGGGGGGGGGGCUCCACCGAGAUC |
| | residue | ACCAAGGCCCCCCUGUCCGCCUCCAUGAUCAAGCGGUACGACGAGCACCACCAGGACCUGACCCUGCUGAAGGCCCUGGUGCGGCAGCAGC |
| | linker | UGCCCGAGAAGUACAAGGAGAUCUUCUUCGACCAGUCCAAGAACGGCUACGCCGGCUACAUCGACGGCGGCGCCUCCCAGGAGGAGUUCUA |
| | and | CAAGUUCAUCAAGCCCAUCCUGGAGAAGAUGGACGGCACCGAGGAGCUGCUGGUGAAGCUGAACCGGGAGGACCUGCUGCGGAAGCAGCGG |
| | HiBit | ACCUUCGACAACGGCUCCAUCCCCCACCAGAUCCACCUGGGCGAGCUGCACGCCAUCCUGCGGCGGCAGGAGGACUUCUACCCCUUCCUGA |
| | tag- | AGGACAACCGGGAGAAGAUCGAGAAGAUCCUGACCUUCCGGAUCCCCUACUACGUGGGCCCCCUGGCCCGGGGCAACUCCCGGUUCGCCUG |
| | SpyCas9 | GAUGACCCGGAAGUCCGAGGAGACCAUCACCCCCUGGAACUUCGAGGAGGUGGUGGACAAGGGCGCCUCCGCCCAGUCCUUCAUCGAGCGG |
| | without | AUGACCAACUUCGACAAGAACCUGCCCAACGAGAAGGUGCUGCCCAAGCACUCCCUGCUGUACGAGUACUUCACCGUGUACAACGAGCUGA |
| | Rec2 | CCAAGGUGAAGUACGUGACCGAGGGCAUGCGGAAGCCCGCCUUCCUGUCCGGCGAGCAGAAGAAGGCCAUCGUGGACCUGCUGUUCAAGAC |
| | domain | CAACCGGAAGGUGACCGUGAAGCAGCUGAAGGAGGACUACUUCAAGAAGAUCGAGUGCUUCGACUCCGUGGAGAUCUCCGGCGUGGAGGAC |
| | (mRNA | CGGUUCAACGCCUCCCUGGGCACCUACCACGACCUGCUGAAGAUCAUCAAGGACAAGGACUUCCUGGACAACGAGGAGAACGAGGACAUCC |
| | V)- | UGGAGGACAUCGUGCUGACCCUGACCCUGUUCGAGGACCGGGAGAUGAUCGAGGAGCGGCUGAAGACCUACGCCCACCUGUUCGACGACAA |
| | ORF | GGUGAUGAAGCAGCUGAAGCGGCGGCGGUACACCGGCUGGGGCCGGCUGUCCCGGAAGCUGAUCAACGGCAUCCGGGACAAGCAGUCCGGC |
| | sequence | AAGACCAUCCUGGACUUCCUGAAGUCCGACGGCUUCGCCAACCGGAACUUCAUGCAGCUGAUCCACGACGACUCCCUGACCUUCAAGGAGG |
| | | ACAUCCAGAAGGCCCAGGUGUCCGGCCAGGGCGACUCCCUGCACGAGCACAUCGCCAACCUGGCCGGCUCCCCCGCCAUCAAGAAGGGCAU |
| | | CCUGCAGACCGUGAAGGUGGUGGACGAGCUGGUGAAGGUGAUGGGCCGGCACAAGCCCGAGAACAUCGUGAUCGAGAUGGCCCGGGAGAAC |
| | | CAGACCACCCAGAAGGGCCAGAAGAACUCCCGGGAGCGGAUGAAGCGGAUCGAGGAGGGCAUCAAGGAGCUGGGCUCCCAGAUCCUGAAGG |
| | | AGCACCCCGUGGAGAACACCCAGCUGCAGAACGAGAAGCUGUACCUGUACUACCUGCAGAACGGCCGGGACAUGUACGUGGACCAGGAGCU |
| | | GGACAUCAACCGGCUGUCCGACUACGACGUGGACCACAUCGUGCCCCAGUCCUUCCUGAAGGACGACUCCAUCGACAACAAGGUGCUGACC |
| | | CGGUCCGACAAGAACCGGGGCAAGUCCGACAACGUGCCCUCCGAGGAGGUGGUGAAGAAGAUGAAGAACUACUGGCGGCAGCUGCUGAACG |
| | | CCAAGCUGAUCACCCAGCGGAAGUUCGACAACCUGACCAAGGCCGAGCGGGGCGGCCUGUCCGAGCUGGACAAGGCCGGCUUCAUCAAGCG |
| | | GCAGCUGGUGGAGACCCGGCAGAUCACCAAGCACGUGGCCCAGAUCCUGGACUCCCGGAUGAACACCAAGUACGACGAGAACGACAAGCUG |
| | | AUCCGGGAGGUGAAGGUGAUCACCCUGAAGUCCAAGCUGGUGUCCGACUUCCGGAAGGACUUCCAGUUCUACAAGGUGCGGGAGAUCAACA |
| | | ACUACCACCACGCCCACGACGCCUACCUGAACGCCGUGGUGGGCACCGCCCUGAUCAAGAAGUACCCCAAGCUGGAGUCCGAGUUCGUGUA |
| | | CGGCGACUACAAGGUGUACGACGUGCGGAAGAUGAUCGCCAAGUCCGAGCAGGAGAUCGGCAAGGCCACCGCCAAGUACUUCUUCUACUCC |
| | | AACAUCAUGAACUUCUUCAAGACCGAGAUCACCCUGGCCAACGGCGAGAUCCGGAAGCGGCCCCUGAUCGAGACCAACGGCGAGACCGGCG |
| | | AGAUCGUGUGGGACAAGGGCCGGGACUUCGCCACCGUGCGGAAGGUGCUGUCCAUGCCCCAGGUGAACAUCGUGAAGAAGACCGAGGUGCA |
| | | GACCGGCGGCUUCUCCAAGGAGUCCAUCCUGCCCAAGCGGAACUCCGACAAGCUGAUCGCCCGGAAGAAGGACUGGGACCCCAAGAAGUAC |
| | | GGCGGCUUCGACUCCCCCACCGUGGCCUACUCCGUGCUGGUGGUGGCCAAGGUGGAGAAGGGCAAGUCCAAGAAGCUGAAGUCCGUGAAGG |
| | | AGCUGCUGGGCAUCACCAUCAUGGAGCGGUCCUCCUUCGAGAAGAACCCCAUCGACUUCCUGGAGGCCAAGGGCUACAAGGAGGUGAAGAA |
| | | GGACCUGAUCAUCAAGCUGCCCAAGUACUCCCUGUUCGAGCUGGAGAACGGCCGGAAGCGGAUGCUGGCCUCCGCCGGCGAGCUGCAGAAG |
| | | GGCAACGAGCUGGCCCUGCCCUCCAAGUACGUGAACUUCCUGUACCUGGCCUCCCACUACGAGAAGCUGAAGGGCUCCCCCGAGGACAACG |
| | | AGCAGAAGCAGCUGUUCGUGGAGCAGCACAAGCACUACCUGGACGAGAUCAUCGAGCAGAUCUCCGAGUUCUCCAAGCGGGUGAUCCUGGC |
| | | CGACGCCAACCUGGACAAGGUGCUGUCCGCCUACAACAAGCACCGGGACAAGCCCAUCCGGGAGCAGGCCGAGAACAUCAUCCACCUGUUC |
| | | ACCCUGACCAACCUGGGCGCCCCCGCCGCCUUCAAGUACUUCGACACCACCAUCGACAAGAAGCGGUACACCUCCACCAAGGAGGUGCUGG |
| | | ACGCCACCCUGAUCCACCAGUCCAUCACCGGCCUGUACGAGACCCGGAUCGACCUGUCCCAGCUGGGGGGCGACGGCGCCCCCGAGUCCGC |
| | | CACCGAGUCCGGCGGCACCUCCACCGAGUCCGAGGGCUCCGCCGGCACCUCCACCGAGUCCGAGGGCUCCGCCGGCUCCGCCGGCUCCACC |
| | | UCCACCGAGGAGGGCACCUCCACCGAGUCCGAGGGCUCCGCCGGCACCUCCACCGAGUCCGAGGGCUCCGCCGGCACCUCCGAGUCCGCCA |
| | | CCGAGUCCGGCGGCACCUCCACCGAGUCCGAGGGCUCCUCCUCCGGUGCCGCCUUCAAGCCCAACCCCAUCAACUACAUCCUGGGCCUGGA |
| | | CAUCGGCAUCGCCUCCGUGGGCUGGGCCAUGGUGGAGAUCGACGAGGAGGAGAACCCCAUCCGGCUGAUCGACCUGGGCGUGCGGGUGUUC |
| | | GAGCGGGCCGAGGUGCCCAAGACCGGCGACUCCCUGGCCAUGGCCCGGCGGCUGGCCCGGUCCGUGCGGCGGCUGACCCGGCGGCGGGCCC |
| | | ACCGGCUGCUGCGGGCCCGGCGGCUGCUGAAGCGGGAGGGCGUGCUGCAGGCCGCCGACUUCGACGAGAACGGCCUGAUCAAGUCCCUGCC |
| | | CAACACCCCCUGGCAGCUGCGGGCCGCCGCCCUGGACCGGAAGCUGACCCCCCUGGAGUGGUCCGCCGUGCUGCUGCACCUGAUCAAGCAC |
| | | CGGGGCUACCUGUCCCAGCGGAAGAACGAGGGCGAGACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGAGAAGGAGUCCGGCCACAUCCGGAACCAGCGGGGCGA |
| | | CUACUCCCACACCUUCUCCCGGAAGGACCUGCAGGCCGAGCUGAUCCUGCUGUUCGAGAAGCAGAAGGAGUUCGGCAACCCCCACGUGUCC |
| | | GGCGGCCUGAAGGAGGGCAUCGAGACCCUGCUGAUGACCCAGCGGCCCGCCCUGUCCGGCGACGCCGUGCAGAAGAUGCUGGGCCACUGCA |
| | | CCUUCGAGCCCGCCGAGCCCAAGGCCGCCAAGAACACCUACACCGCCGAGCGGUUCAUCUGGCUGACCAAGCUGAACAACCUGCGGAUCCU |
| | | GGAGCAGGGCUCCGAGCGGCCCCUGACCGACACCGAGCGGGCCACCCUGAUGGACGAGCCCUACCGGAAGUCCAAGCUGACCUACGCCCAG |
| | | GCCCGGAAGCUGCUGGGCCUGGAGGACACCGCCUUCUUCAAGGGCCUGCGGUACGGCAAGGACAACGCCGAGGCCUCCACCCUGAUGGAGA |
| | | UGAAGGCCUACCACGCCAUCUCCCGGGCCCUGGAGAAGGAGGGCCUGAAGGACAAGAAGUCCCCCCUGAACCUGUCCUCCGAGCUGCAGGA |
| | | CGAGAUCGGCACCGCCUUCUCCCUGUUCAAGACCGACGAGGACAUCACCGGCCGGCUGAAGGACCGGGUGCAGCCCGAGAUCCUGGAGGCC |
| | | CUGCUGAAGCACAUCUCCUUCGACAAGUUCGUGCAGAUCUCCCUGAAGGCCCUGCGGCGGAUCGUGCCCCUGAUGGAGCAGGGCAAGCGGU |
| | | ACGACGAGGCCUGCGCCGAGAUCUACGGCGACCACUACGGCAAGAAGAACACCGAGGAGAAGAUCUACCUGCCCCCCAUCCCCGCCGACGA |
| | | GAUCCGGAACCCCGUGGUGCUGCGGGCCCUGUCCCAGGCCCGGAAGGUGAUCAACGGCGUGGUGCGGCGGUACGGCUCCCCCGCCCGGAUC |
| | | CACAUCGAGACCGCCCGGGAGGUGGGCAAGUCCUUCAAGGACCGGAAGGAGAUCGAGAAGCGGCAGGAGGAGAACCGGAAGGACCGGGAGA |
| | | AGGCCGCCGCCAAGUUCCGGGAGUACUUCCCCAACUUCGUGGGCGAGCCCAAGUCCAAGGACAUCCUGAAGCUGCGGCUGUACGAGCAGCA |
| | | GCACGGCAAGUGCCUGUACUCCGGCAAGGAGAUCAACCUGGUGCGGCUGAACGAGAAGGGCUACGUGGAGAUCGACCACGCCCUGCCCUUC |
| | | UCCCGGACCUGGGACGACUCCUUCAACAACAAGGUGCUGGUGCUGGGCUCCGAGAACCAGAACAAGGGCAACCAGACCCCCUACGAGUACU |
| | | UCAACGGCAAGGACAACUCCCGGGAGUGGCAGGAGUUCAAGGCCCGGGUGGAGACCUCCCGGUUCCCCCGGUCCAAGAAGCAGCGGAUCCU |
| | | GCUGCAGAAGUUCGACGAGGACGGCUUCAAGGAGUGCAACCUGAACGACACCCGGUACGUGAACCGCUUCCUGUGCCAGUUCGUGGCCGAC |
| | | CACAUCCUGCUGACCGGCAAGGGCAAGCGGCGGGUGUUCGCCUCCAACGGCCAGAUCACCAACCUGCUGCGGGGCUUCUGGGGCCUGCGGA |
| | | AGGUGCGGGCCGAGAACGACCGGCACCACGCCCUGGACGCCGUGGUGGUGGCCUGCUCCACCGUGGCCAUGCAGCAGAAGAUCACCCGGUU |
| | | CGUGCGGUACAAGGAGAUGAACGCCUUCGACGGCAAGACCAUCGACAAGGAGACCGGCAAGGUGCUGCACCAGAAGACCCACUUCCCCCAG |
| | | CCCUGGGAGUUCUUCGCCCAGGAGGUGAUGAUCCGGGUGUUCGGCAAGCCCGACGGCAAGCCCGAGUUCGAGGAGGCCGACACCCCCGAGA |
| | | AGCUGCGGACCCUGCUGGCCGAGAAGCUGUCCUCCCGGCCCGAGGCCGUGCACGAGUACGUGACCCCCCUGUUCGUGUCCCGGGCCCCCAA |
| | | CCGGAAGAUGUCCGGCGCCCACAAGGACACCCUGCGGUCCGCCAAGCGGUUCGUGAAGCACAACGAGAAGAUCUCCGUGAAGCGGGUGUGG |
| | | CUGACCGAGAUCAAGCUGGCCGACCUGGAGAACAUGGUGAACUACAAGAACGGCCGGGAGAUCGAGCUGUACGAGGCCCUGAAGGCCCGGC |
| | | UGGAGGCCUACGGCGGCAACGCCAAGCAGGCCUUCGACCCCAAGGACAACCCCUUCUACAAGAAGGGCGGCCAGCUGGUGAAGGCCGUGCG |
| | | GGUGGAGAAGACCCAGGAGUCCGGCGUGCUGCUGAACAAGAAGAACGCCUACACCAUCGCCGACAACGGCGACAUGGUGCGGGUGGACGUG |
| | | UUCUGCAAGGUGGACAAGAAGGGCAAGAACCAGUACUUCAUCGUGCCCAUCUACGCCUGGCAGGUGGCCGAGAACAUCCUGCCCGACAUCG |
| | | ACUGCAAGGGCUACCGGAUCGACGACUCCUACACCUUCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUACCUGGCCUGGCACGACAAGGGCUCCAAGGAGCAGCAG |
| | | UUCCGGAUCUCCACCCAGAACCUGGUGCUGAUCCAGAAGUACCAGGUGAACGAGCUGGGCAAGGAGAUCCGGCCCUGCCGGCUGAAGAAGC |
| | | GGCCCCCCGUGCGGUCCGGAAAGCGGACCGCCGACGGCUCCGGAGGAGGAAGCCCGAAGAAGAAGAGAAAGGUCAGCGAAAGCGCAACACC |
| | | GGAAAGCGUCAGCGGAUGGAGACUGUUCAAGAAGAUCAGCUAG |
| 85 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLGGGGSTEI |
| | fusion- | TKAPLSASMIKRYDEHHQDLTLLKALVRQQLPEKYKEIFFDQSKNGYAGYIDGGASQEEFYKFIKPILEKMDGTEELLVKLNREDLLRKQR |
| | 81 | TFDNGSIPHQIHLGELHAILRRQEDFYPFLKDNREKIEKILTFRIPYYVGPLARGNSRFAWMTRKSEETITPWNFEEVVDKGASAQSFIER |
| | amino | MTNFDKNLPNEKVLPKHSLLYEYFTVYNELTKVKYVTEGMRKPAFLSGEQKKAIVDLLFKTNRKVTVKQLKEDYFKKIECFDSVEISGVED |
| | acid | RFNASLGTYHDLLKIIKDKDFLDNEENEDILEDIVLTLTLFEDREMIEERLKTYAHLFDDKVMKQLKRRRYTGWGRLSRKLINGIRDKQSG |
| | residue | KTILDFLKSDGFANRNFMQLIHDDSLTFKEDIQKAQVSGQGDSLHEHIANLAGSPAIKKGILQTVKVVDELVKVMGRHKPENIVIEMAREN |
| | linker | QTTQKGQKNSRERMKRIEEGIKELGSQILKEHPVENTQLQNEKLYLYYLQNGRDMYVDQELDINRLSDYDVDHIVPQSFLKDDSIDNKVLT |
| | and | RSDKNRGKSDNVPSEEVVKKMKNYWRQLLNAKLITQRKFDNLTKAERGGLSELDKAGFIKRQLVETRQITKHVAQILDSRMNTKYDENDKL |
| | HiBit | IREVKVITLKSKLVSDFRKDFQFYKVREINNYHHAHDAYLNAVVGTALIKKYPKLESEFVYGDYKVYDVRKMIAKSEQEIGKATAKYFFYS |
| | tag- | NIMNFFKTEITLANGEIRKRPLIETNGETGEIVWDKGRDFATVRKVLSMPQVNIVKKTEVQTGGFSKESILPKRNSDKLIARKKDWDPKKY |
| | SpyCas9 | GGFDSPTVAYSVLVVAKVEKGKSKKLKSVKELLGITIMERSSFEKNPIDFLEAKGYKEVKKDLIIKLPKYSLFELENGRKRMLASAGELQK |
| | without | GNELALPSKYVNFLYLASHYEKLKGSPEDNEQKQLFVEQHKHYLDEIIEQISEFSKRVILADANLDKVLSAYNKHRDKPIREQAENIIHLF |
| | Rec2 | TLTNLGAPAAFKYFDTTIDKKRYTSTKEVLDATLIHQSITGLYETRIDLSQLGGDGAPESATESGGTSTESEGSAGTSTESEGSAGSAGST |
| | domain | STEEGTSTESEGSAGTSTESEGSAGTSESATESGGTSTESEGSSSGAAFKPNPINYILGLDIGIASVGWAMVEIDEEENPIRLIDLGVRVF |
| | (mRNA | ERAEVPKTGDSLAMARRLARSVRRLTRRRAHRLLRARRLLKREGVLQAADFDENGLIKSLPNTPWQLRAAALDRKLTPLEWSAVLLHLIKH |
| | V)- | RGYLSQRKNEGETADKELGALLKGVANNAHALQTGDFRTPAELALNKFEKESGHIRNQRGDYSHTFSRKDLQAELILLFEKQKEFGNPHVS |
| | amino | GGLKEGIETLLMTQRPALSGDAVQKMLGHCTFEPAEPKAAKNTYTAERFIWLTKLNNLRILEQGSERPLTDTERATLMDEPYRKSKLTYAQ |
| | acid | ARKLLGLEDTAFFKGLRYGKDNAEASTLMEMKAYHAISRALEKEGLKDKKSPLNLSSELQDEIGTAFSLFKTDEDITGRLKDRVQPEILEA |
| | sequence | LLKHISFDKFVQISLKALRRIVPLMEQGKRYDEACAEIYGDHYGKKNTEEKIYLPPIPADEIRNPVVLRALSQARKVINGVVRRYGSPARI |
| | | HIETAREVGKSFKDRKEIEKRQEENRKDREKAAAKFREYFPNFVGEPKSKDILKLRLYEQQHGKCLYSGKEINLVRLNEKGYVEIDHALPF |
| | | SRTWDDSFNNKVLVLGSENQNKGNQTPYEYFNGKDNSREWQEFKARVETSRFPRSKKQRILLQKFDEDGFKECNLNDTRYVNRFLCQFVAD |
| | | HILLTGKGKRRVFASNGQITNLLRGFWGLRKVRAENDRHHALDAVVVACSTVAMQQKITRFVRYKEMNAFDGKTIDKETGKVLHQKTHFPQ |
| | | PWEFFAQEVMIRVFGKPDGKPEFEEADTPEKLRTLLAEKLSSRPEAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDNPFYKKGGQLVKAVRVEKTQESGVLLNKKNAYTIADNGDMVRVDV |
| | | FCKVDKKGKNQYFIVPIYAWQVAENILPDIDCKGYRIDDSYTFCFSLHKYDLIAFQKDEKSKVEFAYYINCDSSNGRFYLAWHDKGSKEQQ |
| | | FRISTQNLVLIQKYQVNELGKEIRPCRLKKRPPVRSGKRTADGSGGGSPKKKRKVSESATPESVSGWRLFKKIS* |
| 86 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 81 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGGGGGGGGGGGGCUCCACCGAGAUC |
| | residual | ACCAAGGCCCCCCUGUCCGCCUCCAUGAUCAAGCGGUACGACGAGCACCACCAGGACCUGACCCUGCUGAAGGCCCUGGUGCGGCAGCAGC |
| | linker | UGCCCGAGAAGUACAAGGAGAUCUUCUUCGACCAGUCCAAGAACGGCUACGCCGGCUACAUCGACGGCGGCGCCUCCCAGGAGGAGUUCUA |
| | with | CAAGUUCAUCAAGCCCAUCCUGGAGAAGAUGGACGGCACCGAGGAGCUGCUGGUGAAGCUGAACCGGGAGGACCUGCUGCGGAAGCAGCGG |
| | SpyCas9 | ACCUUCGACAACGGCUCCAUCCCCCACCAGAUCCACCUGGGCGAGCUGCACGCCAUCCUGCGGCGGCAGGAGGACUUCUACCCCUUCCUGA |
| | without | AGGACAACCGGGAGAAGAUCGAGAAGAUCCUGACCUUCCGGAUCCCCUACUACGUGGGCCCCCUGGCCCGGGGCAACUCCCGGUUCGCCUG |
| | Rec2 | GAUGACCCGGAAGUCCGAGGAGACCAUCACCCCCUGGAACUUCGAGGAGGUGGUGGACAAGGGCGCCUCCGCCCAGUCCUUCAUCGAGCGG |
| | domain | AUGACCAACUUCGACAAGAACCUGCCCAACGAGAAGGUGCUGCCCAAGCACUCCCUGCUGUACGAGUACUUCACCGUGUACAACGAGCUGA |
| | (mRNA | CCAAGGUGAAGUACGUGACCGAGGGCAUGCGGAAGCCCGCCUUCCUGUCCGGCGAGCAGAAGAAGGCCAUCGUGGACCUGCUGUUCAAGAC |
| | V)- | CAACCGGAAGGUGACCGUGAAGCAGCUGAAGGAGGACUACUUCAAGAAGAUCGAGUGCUUCGACUCCGUGGAGAUCUCCGGCGUGGAGGAC |
| | mRNA | CGGUUCAACGCCUCCCUGGGCACCUACCACGACCUGCUGAAGAUCAUCAAGGACAAGGACUUCCUGGACAACGAGGAGAACGAGGACAUCC |
| | sequence | UGGAGGACAUCGUGCUGACCCUGACCCUGUUCGAGGACCGGGAGAUGAUCGAGGAGCGGCUGAAGACCUACGCCCACCUGUUCGACGACAA |
| | | GGUGAUGAAGCAGCUGAAGCGGCGGCGGUACACCGGCUGGGGCCGGCUGUCCCGGAAGCUGAUCAACGGCAUCCGGGACAAGCAGUCCGGC |
| | | AAGACCAUCCUGGACUUCCUGAAGUCCGACGGCUUCGCCAACCGGAACUUCAUGCAGCUGAUCCACGACGACUCCCUGACCUUCAAGGAGG |
| | | ACAUCCAGAAGGCCCAGGUGUCCGGCCAGGGCGACUCCCUGCACGAGCACAUCGCCAACCUGGCCGGCUCCCCCGCCAUCAAGAAGGGCAU |
| | | CCUGCAGACCGUGAAGGUGGUGGACGAGCUGGUGAAGGUGAUGGGCCGGCACAAGCCCGAGAACAUCGUGAUCGAGAUGGCCCGGGAGAAC |
| | | CAGACCACCCAGAAGGGCCAGAAGAACUCCCGGGAGCGGAUGAAGCGGAUCGAGGAGGGCAUCAAGGAGCUGGGCUCCCAGAUCCUGAAGG |
| | | AGCACCCCGUGGAGAACACCCAGCUGCAGAACGAGAAGCUGUACCUGUACUACCUGCAGAACGGCCGGGACAUGUACGUGGACCAGGAGCU |
| | | GGACAUCAACCGGCUGUCCGACUACGACGUGGACCACAUCGUGCCCCAGUCCUUCCUGAAGGACGACUCCAUCGACAACAAGGUGCUGACC |
| | | CGGUCCGACAAGAACCGGGGCAAGUCCGACAACGUGCCCUCCGAGGAGGUGGUGAAGAAGAUGAAGAACUACUGGCGGCAGCUGCUGAACG |
| | | CCAAGCUGAUCACCCAGCGGAAGUUCGACAACCUGACCAAGGCCGAGCGGGGCGGCCUGUCCGAGCUGGACAAGGCCGGCUUCAUCAAGCG |
| | | GCAGCUGGUGGAGACCCGGCAGAUCACCAAGCACGUGGCCCAGAUCCUGGACUCCCGGAUGAACACCAAGUACGACGAGAACGACAAGCUG |
| | | AUCCGGGAGGUGAAGGUGAUCACCCUGAAGUCCAAGCUGGUGUCCGACUUCCGGAAGGACUUCCAGUUCUACAAGGUGCGGGAGAUCAACA |
| | | ACUACCACCACGCCCACGACGCCUACCUGAACGCCGUGGUGGGCACCGCCCUGAUCAAGAAGUACCCCAAGCUGGAGUCCGAGUUCGUGUA |
| | | CGGCGACUACAAGGUGUACGACGUGCGGAAGAUGAUCGCCAAGUCCGAGCAGGAGAUCGGCAAGGCCACCGCCAAGUACUUCUUCUACUCC |
| | | AACAUCAUGAACUUCUUCAAGACCGAGAUCACCCUGGCCAACGGCGAGAUCCGGAAGCGGCCCCUGAUCGAGACCAACGGCGAGACCGGCG |
| | | AGAUCGUGUGGGACAAGGGCCGGGACUUCGCCACCGUGCGGAAGGUGCUGUCCAUGCCCCAGGUGAACAUCGUGAAGAAGACCGAGGUGCA |
| | | GACCGGCGGCUUCUCCAAGGAGUCCAUCCUGCCCAAGCGGAACUCCGACAAGCUGAUCGCCCGGAAGAAGGACUGGGACCCCAAGAAGUAC |
| | | GGCGGCUUCGACUCCCCCACCGUGGCCUACUCCGUGCUGGUGGUGGCCAAGGUGGAGAAGGGCAAGUCCAAGAAGCUGAAGUCCGUGAAGG |
| | | AGCUGCUGGGCAUCACCAUCAUGGAGCGGUCCUCCUUCGAGAAGAACCCCAUCGACUUCCUGGAGGCCAAGGGCUACAAGGAGGUGAAGAA |
| | | GGACCUGAUCAUCAAGCUGCCCAAGUACUCCCUGUUCGAGCUGGAGAACGGCCGGAAGCGGAUGCUGGCCUCCGCCGGCGAGCUGCAGAAG |
| | | GGCAACGAGCUGGCCCUGCCCUCCAAGUACGUGAACUUCCUGUACCUGGCCUCCCACUACGAGAAGCUGAAGGGCUCCCCCGAGGACAACG |
| | | AGCAGAAGCAGCUGUUCGUGGAGCAGCACAAGCACUACCUGGACGAGAUCAUCGAGCAGAUCUCCGAGUUCUCCAAGCGGGUGAUCCUGGC |
| | | CGACGCCAACCUGGACAAGGUGCUGUCCGCCUACAACAAGCACCGGGACAAGCCCAUCCGGGAGCAGGCCGAGAACAUCAUCCACCUGUUC |
| | | ACCCUGACCAACCUGGGCGCCCCCGCCGCCUUCAAGUACUUCGACACCACCAUCGACAAGAAGCGGUACACCUCCACCAAGGAGGUGCUGG |
| | | ACGCCACCCUGAUCCACCAGUCCAUCACCGGCCUGUACGAGACCCGGAUCGACCUGUCCCAGCUGGGCGGCGACGGCGCCCCCGAGUCCGC |
| | | CACCGAGUCCGGCGGCACCUCCACCGAGUCCGAGGGCUCCGCCGGCACCUCCACCGAGUCCGAGGGCUCCGCCGGCUCCGCCGGCUCCACC |
| | | UCCACCGAGGAGGGCACCUCCACCGAGUCCGAGGGCUCCGCCGGCACCUCCACCGAGUCCGAGGGCUCCGCCGGCACCUCCGAGUCCGCCA |
| | | CCGAGUCCGGCGGCACCUCCACCGAGUCCGAGGGCUCCUCCUCCGGUGCCGCCUUCAAGCCCAACCCCAUCAACUACAUCCUGGGCCUGGA |
| | | CAUCGGCAUCGCCUCCGUGGGCUGGGCCAUGGUGGAGAUCGACGAGGAGGAGAACCCCAUCCGGCUGAUCGACCUGGGCGUGCGGGUGUUC |
| | | GAGCGGGCCGAGGUGCCCAAGACCGGCGACUCCCUGGCCAUGGCCCGGCGGCUGGCCCGGUCCGUGCGGCGGCUGACCCGGCGGCGGGCCC |
| | | ACCGGCUGCUGCGGGCCCGGCGGCUGCUGAAGCGGGAGGGCGUGCUGCAGGCCGCCGACUUCGACGAGAACGGCCUGAUCAAGUCCCUGCC |
| | | CAACACCCCCUGGCAGCUGCGGGCCGCCGCCCUGGACCGGAAGCUGACCCCCCUGGAGUGGUCCGCCGUGCUGCUGCACCUGAUCAAGCAC |
| | | CGGGGCUACCUGUCCCAGCGGAAGAACGAGGGCGAGACCGCCGACAAGGAGCUGGGCGCCCUGCUGAAGGGCGUGGCCAACAACGCCCACG |
| | | CCCUGCAGACCGGCGACUUCCGGACCCCCGCCGAGCUGGCCCUGAACAAGUUCGAGAAGGAGUCCGGCCACAUCCGGAACCAGCGGGGCGA |
| | | CUACUCCCACACCUUCUCCCGGAAGGACCUGCAGGCCGAGCUGAUCCUGCUGUUCGAGAAGCAGAAGGAGUUCGGCAACCCCCACGUGUCC |
| | | GGCGGCCUGAAGGAGGGCAUCGAGACCCUGCUGAUGACCCAGCGGCCCGCCCUGUCCGGCGACGCCGUGCAGAAGAUGCUGGGCCACUGCA |
| | | CCUUCGAGCCCGCCGAGCCCAAGGCCGCCAAGAACACCUACACCGCCGAGCGGUUCAUCUGGCUGACCAAGCUGAACAACCUGCGGAUCCU |
| | | GGAGCAGGGCUCCGAGCGGCCCCUGACCGACACCGAGCGGGCCACCCUGAUGGACGAGCCCUACCGGAAGUCCAAGCUGACCUACGCCCAG |
| | | GCCCGGAAGCUGCUGGGCCUGGAGGACACCGCCUUCUUCAAGGGCCUGCGGUACGGCAAGGACAACGCCGAGGCCUCCACCCUGAUGGAGA |
| | | UGAAGGCCUACCACGCCAUCUCCCGGGCCCUGGAGAAGGAGGGCCUGAAGGACAAGAAGUCCCCCCUGAACCUGUCCUCCGAGCUGCAGGA |
| | | CGAGAUCGGCACCGCCUUCUCCCUGUUCAAGACCGACGAGGACAUCACCGGCCGGCUGAAGGACCGGGUGCAGCCCGAGAUCCUGGAGGCC |
| | | CUGCUGAAGCACAUCUCCUUCGACAAGUUCGUGCAGAUCUCCCUGAAGGCCCUGCGGCGGAUCGUGCCCCUGAUGGAGCAGGGCAAGCGGU |
| | | ACGACGAGGCCUGCGCCGAGAUCUACGGCGACCACUACGGCAAGAAGAACACCGAGGAGAAGAUCUACCUGCCCCCCAUCCCCGCCGACGA |
| | | GAUCCGGAACCCCGUGGUGCUGCGGGCCCUGUCCCAGGCCCGGAAGGUGAUCAACGGCGUGGUGCGGCGGUACGGCUCCCCCGCCCGGAUC |
| | | CACAUCGAGACCGCCCGGGAGGUGGGCAAGUCCUUCAAGGACCGGAAGGAGAUCGAGAAGCGGCAGGAGGAGAACCGGAAGGACCGGGAGA |
| | | AGGCCGCCGCCAAGUUCCGGGAGUACUUCCCCAACUUCGUGGGCGAGCCCAAGUCCAAGGACAUCCUGAAGCUGCGGCUGUACGAGCAGCA |
| | | GCACGGCAAGUGCCUGUACUCCGGCAAGGAGAUCAACCUGGUGCGGCUGAACGAGAAGGGCUACGUGGAGAUCGACCACGCCCUGCCCUUC |
| | | UCCCGGACCUGGGACGACUCCUUCAACAACAAGGUGCUGGUGCUGGGCUCCGAGAACCAGAACAAGGGCAACCAGACCCCCUACGAGUACU |
| | | UCAACGGCAAGGACAACUCCCGGGAGUGGCAGGAGUUCAAGGCCCGGGUGGAGACCUCCCGGUUCCCCCGGUCCAAGAAGCAGCGGAUCCU |
| | | GCUGCAGAAGUUCGACGAGGACGGCUUCAAGGAGUGCAACCUGAACGACACCCGGUACGUGAACCGCUUCCUGUGCCAGUUCGUGGCCGAC |
| | | CACAUCCUGCUGACCGGCAAGGGCAAGCGGCGGGUGUUCGCCUCCAACGGCCAGAUCACCAACCUGCUGCGGGGCUUCUGGGGCCUGCGGA |
| | | AGGUGCGGGCCGAGAACGACCGGCACCACGCCCUGGACGCCGUGGUGGUGGCCUGCUCCACCGUGGCCAUGCAGCAGAAGAUCACCCGGUU |
| | | CGUGCGGUACAAGGAGAUGAACGCCUUCGACGGCAAGACCAUCGACAAGGAGACCGGCAAGGUGCUGCACCAGAAGACCCACUUCCCCCAG |
| | | CCCUGGGAGUUCUUCGCCCAGGAGGUGAUGAUCCGGGUGUUCGGCAAGCCCGACGGCAAGCCCGAGUUCGAGGAGGCCGACACCCCCGAGA |
| | | AGCUGCGGACCCUGCUGGCCGAGAAGCUGUCCUCCCGGCCCGAGGCCGUGCACGAGUACGUGACCCCCCUGUUCGUGUCCCGGGCCCCCAA |
| | | CCGGAAGAUGUCCGGCGCCCACAAGGACACCCUGCGGUCCGCCAAGCGGUUCGUGAAGCACAACGAGAAGAUCUCCGUGAAGCGGGUGUGG |
| | | CUGACCGAGAUCAAGCUGGCCGACCUGGAGAACAUGGUGAACUACAAGAACGGCCGGGAGAUCGAGCUGUACGAGGCCCUGAAGGCCCGGC |
| | | UGGAGGCCUACGGCGGCAACGCCAAGCAGGCCUUCGACCCCAAGGACAACCCCUUCUACAAGAAGGGCGGCCAGCUGGUGAAGGCCGUGCG |
| | | GGUGGAGAAGACCCAGGAGUCCGGCGUGCUGCUGAACAAGAAGAACGCCUACACCAUCGCCGACAACGGCGACAUGGUGCGGGUGGACGUG |
| | | UUCUGCAAGGUGGACAAGAAGGGCAAGAACCAGUACUUCAUCGUGCCCAUCUACGCCUGGCAGGUGGCCGAGAACAUCCUGCCCGACAUCG |
| | | ACUGCAAGGGCUACCGGAUCGACGACUCCUACACCUUCUGCUUCUCCCUGCACAAGUACGACCUGAUCGCCUUCCAGAAGGACGAGAAGUC |
| | | CAAGGUGGAGUUCGCCUACUACAUCAACUGCGACUCCUCCAACGGCCGGUUCUACCUGGCCUGGCACGACAAGGGCUCCAAGGAGCAGCAG |
| | | UUCCGGAUCUCCACCCAGAACCUGGUGCUGAUCCAGAAGUACCAGGUGAACGAGCUGGGCAAGGAGAUCCGGCCCUGCCGGCUGAAGAAGC |
| | | GGCCCCCCGUGCGGUCCGGAAAGCGGACCGCCGACGGCUCCGGAGGAGGAAGCCCGAAGAAGAAGAGAAAGGUCUAG |
| 87 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLGGGGSTEI |
| | fusion- | TKAPLSASMIKRYDEHHQDLTLLKALVRQQLPEKYKEIFFDQSKNGYAGYIDGGASQEEFYKFIKPILEKMDGTEELLVKLNREDLLRKQR |
| | 81 | TFDNGSIPHQIHLGELHAILRRQEDFYPFLKDNREKIEKILTFRIPYYVGPLARGNSRFAWMTRKSEETITPWNFEEVVDKGASAQSFIER |
| | amino | MTNFDKNLPNEKVLPKHSLLYEYFTVYNELTKVKYVTEGMRKPAFLSGEQKKAIVDLLFKTNRKVTVKQLKEDYFKKIECFDSVEISGVED |
| | acid | RFNASLGTYHDLLKIIKDKDFLDNEENEDILEDIVLTLTLFEDREMIEERLKTYAHLFDDKVMKQLKRRRYTGWGRLSRKLINGIRDKQSG |
| | residual | KTILDFLKSDGFANRNFMQLIHDDSLTFKEDIQKAQVSGQGDSLHEHIANLAGSPAIKKGILQTVKVVDELVKVMGRHKPENIVIEMAREN |
| | linker | QTTQKGQKNSRERMKRIEEGIKELGSQILKEHPVENTQLQNEKLYLYYLQNGRDMYVDQELDINRLSDYDVDHIVPQSFLKDDSIDNKVLT |
| | with | RSDKNRGKSDNVPSEEVVKKMKNYWRQLLNAKLITQRKFDNLTKAERGGLSELDKAGFIKRQLVETRQITKHVAQILDSRMNTKYDENDKL |
| | SpyCas9 | IREVKVITLKSKLVSDFRKDFQFYKVREINNYHHAHDAYLNAVVGTALIKKYPKLESEFVYGDYKVYDVRKMIAKSEQEIGKATAKYFFYS |
| | without | NIMNFFKTEITLANGEIRKRPLIETNGETGEIVWDKGRDFATVRKVLSMPQVNIVKKTEVQTGGFSKESILPKRNSDKLIARKKDWDPKKY |
| | Rec2 | GGFDSPTVAYSVLVVAKVEKGKSKKLKSVKELLGITIMERSSFEKNPIDFLEAKGYKEVKKDLIIKLPKYSLFELENGRKRMLASAGELQK |
| | domain | GNELALPSKYVNFLYLASHYEKLKGSPEDNEQKQLFVEQHKHYLDEIIEQISEFSKRVILADANLDKVLSAYNKHRDKPIREQAENIIHLF |
| | (mRNA | TLTNLGAPAAFKYFDTTIDKKRYTSTKEVLDATLIHQSITGLYETRIDLSQLGGDGAPESATESGGTSTESEGSAGTSTESEGSAGSAGST |
| | V)- | STEEGTSTESEGSAGTSTESEGSAGTSESATESGGTSTESEGSSSGAAFKPNPINYILGLDIGIASVGWAMVEIDEEENPIRLIDLGVRVF |
| | amino | ERAEVPKTGDSLAMARRLARSVRRLTRRRAHRLLRARRLLKREGVLQAADFDENGLIKSLPNTPWQLRAAALDRKLTPLEWSAVLLHLIKH |
| | acid | RGYLSQRKNEGETADKELGALLKGVANNAHALQTGDFRTPAELALNKFEKESGHIRNQRGDYSHTFSRKDLQAELILLFEKQKEFGNPHVS |
| | sequence | GGLKEGIETLLMTQRPALSGDAVQKMLGHCTFEPAEPKAAKNTYTAERFIWLTKLNNLRILEQGSERPLTDTERATLMDEPYRKSKLTYAQ |
| | | ARKLLGLEDTAFFKGLRYGKDNAEASTLMEMKAYHAISRALEKEGLKDKKSPLNLSSELQDEIGTAFSLFKTDEDITGRLKDRVQPEILEA |
| | | LLKHISFDKFVQISLKALRRIVPLMEQGKRYDEACAEIYGDHYGKKNTEEKIYLPPIPADEIRNPVVLRALSQARKVINGVVRRYGSPARI |
| | | HIETAREVGKSFKDRKEIEKRQEENRKDREKAAAKFREYFPNFVGEPKSKDILKLRLYEQQHGKCLYSGKEINLVRLNEKGYVEIDHALPF |
| | | SRTWDDSFNNKVLVLGSENQNKGNQTPYEYFNGKDNSREWQEFKARVETSRFPRSKKQRILLQKFDEDGFKECNLNDTRYVNRFLCQFVAD |
| | | HILLTGKGKRRVFASNGQITNLLRGFWGLRKVRAENDRHHALDAVVVACSTVAMQQKITRFVRYKEMNAFDGKTIDKETGKVLHQKTHFPQ |
| | | PWEFFAQEVMIRVFGKPDGKPEFEEADTPEKLRTLLAEKLSSRPEAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDNPFYKKGGQLVKAVRVEKTQESGVLLNKKNAYTIADNGDMVRVDV |
| | | FCKVDKKGKNQYFIVPIYAWQVAENILPDIDCKGYRIDDSYTFCFSLHKYDLIAFQKDEKSKVEFAYYINCDSSNGRFYLAWHDKGSKEQQ |
| | | FRISTQNLVLIQKYQVNELGKEIRPCRLKKRPPVRSGKRTADGSGGGSPKKKRKV* |
| 88 | Cas9- | GGGAAGCUCAGAAUAAACGCUCAACUUUGGCCGGAUCUGCCACCAUGGACAAGAAGUACUCCAUCGGCCUGGACAUCGGCACCAACUCCGU |
| | Cas9 | GGGCUGGGCCGUGAUCACCGACGAGUACAAGGUGCCCUCCAAGAAGUUCAAGGUGCUGGGCAACACCGACCGGCACUCCAUCAAGAAGAAC |
| | fusion- | CUGAUCGGCGCCCUGCUGUUCGACUCCGGCGAGACCGCCGAGGCCACCCGGCUGAAGCGGACCGCCCGGCGGCGGUACACCCGGCGGAAGA |
| | 81 | ACCGGAUCUGCUACCUGCAGGAGAUCUUCUCCAACGAGAUGGCCAAGGUGGACGACUCCUUCUUCCACCGGCUGGAGGAGUCCUUCCUGGU |
| | amino | GGAGGAGGACAAGAAGCACGAGCGGCACCCCAUCUUCGGCAACAUCGUGGACGAGGUGGCCUACCACGAGAAGUACCCCACCAUCUACCAC |
| | acid | CUGCGGAAGAAGCUGGUGGACUCCACCGACAAGGCCGACCUGCGGCUGAUCUACCUGGCCCUGGCCCACAUGAUCAAGUUCCGGGGCCACU |
| | residue | UCCUGAUCGAGGGCGACCUGAACCCCGACAACUCCGACGUGGACAAGCUGUUCAUCCAGCUGGUGCAGACCUACAACCAGCUGUUCGAGGA |
| | linker | GAACCCCAUCAACGCCUCCGGCGUGGACGCCAAGGCCAUCCUGUCCGCCCGGCUGUCCAAGUCCCGGCGGCUGGAGAACCUGAUCGCCCAG |
| | and | CUGCCCGGCGAGAAGAAGAACGGCCUGUUCGGCAACCUGAUCGCCCUGUCCCUGGGCCUGACCCCCAACUUCAAGUCCAACUUCGACCUGG |
| | HiBit | CCGAGGACGCCAAGCUGCAGCUGUCCAAGGACACCUACGACGACGACCUGGACAACCUGCUGGCCCAGAUCGGCGACCAGUACGCCGACCU |
| | tag- | GUUCCUGGCCGCCAAGAACCUGUCCGACGCCAUCCUGCUGUCCGACAUCCUGCGGGUGAACACCGAGAUCACCAAGGCCCCCCUGUCCGCC |
| | SpyCas9 | UCCAUGAUCAAGCGGUACGACGAGCACCACCAGGACCUGACCCUGCUGAAGGCCCUGGUGCGGCAGCAGCUGCCCGAGAAGUACAAGGAGA |
| | without | UCUUCUUCGACCAGUCCAAGAACGGCUACGCCGGCUACAUCGACGGCGGCGCCUCCCAGGAGGAGUUCUACAAGUUCAUCAAGCCCAUCCU |
| | Rec3 | GGAGAAGAUGGACGGCACCGAGGAGCUGCUGGUGAAGCUGAACCGGGAGGACCUGCUGCGGAAGCAGCGGACCUUCGACAACGGCUCCAUC |
| | domain | CCCCACCAGAUCCACCUGGGCGAGCUGCACGCCAUCCUGCGGCGGCAGGAGGACUUCUACCCCUUCCUGAAGGACAACCGGGAGAAGAUCG |
| | (mRNA | AGAAGAUCCUGACCUUCCGGAUCCCCUACUACGUGGGCCCCCUGGCCCGGGGCAACUCCCGGUUCGCCUGGAUGACCCGGAAGUCCGAGGA |
| | W)- | GACCAUCACCCCCUGGAACUUCGAGGAGGUGGUGGACAAGGGCGCCUCCGCCCAGUCCUUCAUCGAGCGGAUGACCGGCGGCUCCUCCGGC |
| | mRNA | CAGGGCGACUCCCUGCACGAGCACAUCGCCAACCUGGCCGGCUCCCCCGCCAUCAAGAAGGGCAUCCUGCAGACCGUGAAGGUGGUGGACG |
| | sequence | AGCUGGUGAAGGUGAUGGGCCGGCACAAGCCCGAGAACAUCGUGAUCGAGAUGGCCCGGGAGAACCAGACCACCCAGAAGGGCCAGAAGAA |
| | | CUCCCGGGAGCGGAUGAAGCGGAUCGAGGAGGGCAUCAAGGAGCUGGGCUCCCAGAUCCUGAAGGAGCACCCCGUGGAGAACACCCAGCUG |
| | | CAGAACGAGAAGCUGUACCUGUACUACCUGCAGAACGGCCGGGACAUGUACGUGGACCAGGAGCUGGACAUCAACCGGCUGUCCGACUACG |
| | | ACGUGGACCACAUCGUGCCCCAGUCCUUCCUGAAGGACGACUCCAUCGACAACAAGGUGCUGACCCGGUCCGACAAGAACCGGGGCAAGUC |
| | | CGACAACGUGCCCUCCGAGGAGGUGGUGAAGAAGAUGAAGAACUACUGGCGGCAGCUGCUGAACGCCAAGCUGAUCACCCAGCGGAAGUUC |
| | | GACAACCUGACCAAGGCCGAGCGGGGCGGCCUGUCCGAGCUGGACAAGGCCGGCUUCAUCAAGCGGCAGCUGGUGGAGACCCGGCAGAUCA |
| | | CCAAGCACGUGGCCCAGAUCCUGGACUCCCGGAUGAACACCAAGUACGACGAGAACGACAAGCUGAUCCGGGAGGUGAAGGUGAUCACCCU |
| | | GAAGUCCAAGCUGGUGUCCGACUUCCGGAAGGACUUCCAGUUCUACAAGGUGCGGGAGAUCAACAACUACCACCACGCCCACGACGCCUAC |
| | | CUGAACGCCGUGGUGGGCACCGCCCUGAUCAAGAAGUACCCCAAGCUGGAGUCCGAGUUCGUGUACGGCGACUACAAGGUGUACGACGUGC |
| | | GGAAGAUGAUCGCCAAGUCCGAGCAGGAGAUCGGCAAGGCCACCGCCAAGUACUUCUUCUACUCCAACAUCAUGAACUUCUUCAAGACCGA |
| | | GAUCACCCUGGCCAACGGCGAGAUCCGGAAGCGGCCCCUGAUCGAGACCAACGGCGAGACCGGCGAGAUCGUGUGGGACAAGGGCCGGGAC |
| | | UUCGCCACCGUGCGGAAGGUGCUGUCCAUGCCCCAGGUGAACAUCGUGAAGAAGACCGAGGUGCAGACCGGCGGCUUCUCCAAGGAGUCCA |
| | | UCCUGCCCAAGCGGAACUCCGACAAGCUGAUCGCCCGGAAGAAGGACUGGGACCCCAAGAAGUACGGCGGCUUCGACUCCCCCACCGUGGC |
| | | CUACUCCGUGCUGGUGGUGGCCAAGGUGGAGAAGGGCAAGUCCAAGAAGCUGAAGUCCGUGAAGGAGCUGCUGGGCAUCACCAUCAUGGAG |
| | | CGGUCCUCCUUCGAGAAGAACCCCAUCGACUUCCUGGAGGCCAAGGGCUACAAGGAGGUGAAGAAGGACCUGAUCAUCAAGCUGCCCAAGU |
| | | ACUCCCUGUUCGAGCUGGAGAACGGCCGGAAGCGGAUGCUGGCCUCCGCCGGCGAGCUGCAGAAGGGCAACGAGCUGGCCCUGCCCUCCAA |
| | | GUACGUGAACUUCCUGUACCUGGCCUCCCACUACGAGAAGCUGAAGGGCUCCCCCGAGGACAACGAGCAGAAGCAGCUGUUCGUGGAGCAG |
| | | CACAAGCACUACCUGGACGAGAUCAUCGAGCAGAUCUCCGAGUUCUCCAAGCGGGUGAUCCUGGCCGACGCCAACCUGGACAAGGUGCUGU |
| | | CCGCCUACAACAAGCACCGGGACAAGCCCAUCCGGGAGCAGGCCGAGAACAUCAUCCACCUGUUCACCCUGACCAACCUGGGCGCCCCCGC |
| | | CGCCUUCAAGUACUUCGACACCACCAUCGACAAGAAGCGGUACACCUCCACCAAGGAGGUGCUGGACGCCACCCUGAUCCACCAGUCCAUC |
| | | ACCGGCCUGUACGAGACCCGGAUCGACCUGUCCCAGCUGGGGGGCGACGGCGCCCCCGAGUCCGCCACCGAGUCCGGCGGCACCUCCACCG |
| | | AGUCCGAGGGCUCCGCCGGCACCUCCACCGAGUCCGAGGGCUCCGCCGGCUCCGCCGGCUCCACCUCCACCGAGGAGGGCACCUCCACCGA |
| | | GUCCGAGGGCUCCGCCGGCACCUCCACCGAGUCCGAGGGCUCCGCCGGCACCUCCGAGUCCGCCACCGAGUCCGGCGGCACCUCCACCGAG |
| | | UCCGAGGGCUCCUCCUCCGGUGCCGCCUUCAAGCCCAACCCCAUCAACUACAUCCUGGGCCUGGACAUCGGCAUCGCCUCCGUGGGCUGGG |
| | | CCAUGGUGGAGAUCGACGAGGAGGAGAACCCCAUCCGGCUGAUCGACCUGGGCGUGCGGGUGUUCGAGCGGGCCGAGGUGCCCAAGACCGG |
| | | CGACUCCCUGGCCAUGGCCCGGCGGCUGGCCCGGUCCGUGCGGCGGCUGACCCGGCGGCGGGCCCACCGGCUGCUGCGGGCCCGGCGGCUG |
| | | CUGAAGCGGGAGGGCGUGCUGCAGGCCGCCGACUUCGACGAGAACGGCCUGAUCAAGUCCCUGCCCAACACCCCCUGGCAGCUGCGGGCCG |
| | | CCGCCCUGGACCGGAAGCUGACCCCCCUGGAGUGGUCCGCCGUGCUGCUGCACCUGAUCAAGCACCGGGGCUACCUGUCCCAGCGGAAGAA |
| | | CGAGGGCGAGACCGCCGACAAGGAGCUGGGCGCCCUGCUGAAGGGCGUGGCCAACAACGCCCACGCCCUGCAGACCGGCGACUUCCGGACC |
| | | CCCGCCGAGCUGGCCCUGAACAAGUUCGAGAAGGAGUCCGGCCACAUCCGGAACCAGCGGGGCGACUACUCCCACACCUUCUCCCGGAAGG |
| | | ACCUGCAGGCCGAGCUGAUCCUGCUGUUCGAGAAGCAGAAGGAGUUCGGCAACCCCCACGUGUCCGGCGGCCUGAAGGAGGGCAUCGAGAC |
| | | CCUGCUGAUGACCCAGCGGCCCGCCCUGUCCGGCGACGCCGUGCAGAAGAUGCUGGGCCACUGCACCUUCGAGCCCGCCGAGCCCAAGGCC |
| | | GCCAAGAACACCUACACCGCCGAGCGGUUCAUCUGGCUGACCAAGCUGAACAACCUGCGGAUCCUGGAGCAGGGCUCCGAGCGGCCCCUGA |
| | | CCGACACCGAGCGGGCCACCCUGAUGGACGAGCCCUACCGGAAGUCCAAGCUGACCUACGCCCAGGCCCGGAAGCUGCUGGGCCUGGAGGA |
| | | CACCGCCUUCUUCAAGGGCCUGCGGUACGGCAAGGACAACGCCGAGGCCUCCACCCUGAUGGAGAUGAAGGCCUACCACGCCAUCUCCCGG |
| | | GCCCUGGAGAAGGAGGGCCUGAAGGACAAGAAGUCCCCCCUGAACCUGUCCUCCGAGCUGCAGGACGAGAUCGGCACCGCCUUCUCCCUGU |
| | | UCAAGACCGACGAGGACAUCACCGGCCGGCUGAAGGACCGGGUGCAGCCCGAGAUCCUGGAGGCCCUGCUGAAGCACAUCUCCUUCGACAA |
| | | GUUCGUGCAGAUCUCCCUGAAGGCCCUGCGGCGGAUCGUGCCCCUGAUGGAGCAGGGCAAGCGGUACGACGAGGCCUGCGCCGAGAUCUAC |
| | | GGCGACCACUACGGCAAGAAGAACACCGAGGAGAAGAUCUACCUGCCCCCCAUCCCCGCCGACGAGAUCCGGAACCCCGUGGUGCUGCGGG |
| | | CCCUGUCCCAGGCCCGGAAGGUGAUCAACGGCGUGGUGCGGCGGUACGGCUCCCCCGCCCGGAUCCACAUCGAGACCGCCCGGGAGGUGGG |
| | | CAAGUCCUUCAAGGACCGGAAGGAGAUCGAGAAGCGGCAGGAGGAGAACCGGAAGGACCGGGAGAAGGCCGCCGCCAAGUUCCGGGAGUAC |
| | | UUCCCCAACUUCGUGGGCGAGCCCAAGUCCAAGGACAUCCUGAAGCUGCGGCUGUACGAGCAGCAGCACGGCAAGUGCCUGUACUCCGGCA |
| | | AGGAGAUCAACCUGGUGCGGCUGAACGAGAAGGGCUACGUGGAGAUCGACCACGCCCUGCCCUUCUCCCGGACCUGGGACGACUCCUUCAA |
| | | CAACAAGGUGCUGGUGCUGGGCUCCGAGAACCAGAACAAGGGCAACCAGACCCCCUACGAGUACUUCAACGGCAAGGACAACUCCCGGGAG |
| | | UGGCAGGAGUUCAAGGCCCGGGUGGAGACCUCCCGGUUCCCCCGGUCCAAGAAGCAGCGGAUCCUGCUGCAGAAGUUCGACGAGGACGGCU |
| | | UCAAGGAGUGCAACCUGAACGACACCCGGUACGUGAACCGCUUCCUGUGCCAGUUCGUGGCCGACCACAUCCUGCUGACCGGCAAGGGCAA |
| | | GCGGCGGGUGUUCGCCUCCAACGGCCAGAUCACCAACCUGCUGCGGGGCUUCUGGGGCCUGCGGAAGGUGCGGGCCGAGAACGACCGGCAC |
| | | CACGCCCUGGACGCCGUGGUGGUGGCCUGCUCCACCGUGGCCAUGCAGCAGAAGAUCACCCGGUUCGUGCGGUACAAGGAGAUGAACGCCU |
| | | UCGACGGCAAGACCAUCGACAAGGAGACCGGCAAGGUGCUGCACCAGAAGACCCACUUCCCCCAGCCCUGGGAGUUCUUCGCCCAGGAGGU |
| | | GAUGAUCCGGGUGUUCGGCAAGCCCGACGGCAAGCCCGAGUUCGAGGAGGCCGACACCCCCGAGAAGCUGCGGACCCUGCUGGCCGAGAAG |
| | | CUGUCCUCCCGGCCCGAGGCCGUGCACGAGUACGUGACCCCCCUGUUCGUGUCCCGGGCCCCCAACCGGAAGAUGUCCGGCGCCCACAAGG |
| | | ACACCCUGCGGUCCGCCAAGCGGUUCGUGAAGCACAACGAGAAGAUCUCCGUGAAGCGGGUGUGGCUGACCGAGAUCAAGCUGGCCGACCU |
| | | GGAGAACAUGGUGAACUACAAGAACGGCCGGGAGAUCGAGCUGUACGAGGCCCUGAAGGCCCGGCUGGAGGCCUACGGCGGCAACGCCAAG |
| | | CAGGCCUUCGACCCCAAGGACAACCCCUUCUACAAGAAGGGCGGCCAGCUGGUGAAGGCCGUGCGGGUGGAGAAGACCCAGGAGUCCGGCG |
| | | UGCUGCUGAACAAGAAGAACGCCUACACCAUCGCCGACAACGGCGACAUGGUGCGGGUGGACGUGUUCUGCAAGGUGGACAAGAAGGGCAA |
| | | GAACCAGUACUUCAUCGUGCCCAUCUACGCCUGGCAGGUGGCCGAGAACAUCCUGCCCGACAUCGACUGCAAGGGCUACCGGAUCGACGAC |
| | | UCCUACACCUUCUGCUUCUCCCUGCACAAGUACGACCUGAUCGCCUUCCAGAAGGACGAGAAGUCCAAGGUGGAGUUCGCCUACUACAUCA |
| | | ACUGCGACUCCUCCAACGGCCGGUUCUACCUGGCCUGGCACGACAAGGGCUCCAAGGAGCAGCAGUUCCGGAUCUCCACCCAGAACCUGGU |
| | | GCUGAUCCAGAAGUACCAGGUGAACGAGCUGGGCAAGGAGAUCCGGCCCUGCCGGCUGAAGAAGCGGCCCCCCGUGCGGUCCGGAAAGCGG |
| | | ACCGCCGACGGCUCCGGAGGAGGAAGCCCGAAGAAGAAGAGAAAGGUCAGCGAAAGCGCAACACCGGAAAGCGUCAGCGGAUGGAGACUGU |
| | | UCAAGAAGAUCAGCUAGCUAGCACCAGCCUCAAGAACACCCGAAUGGAGUCUCUAAGCUACAUAAUACCAACUUACACUUUACAAAAUGUU |
| | | GUCCCCCAAAAUGUAGCCAUUCGUAUCUGCUCCUAAUAAAAAGAAAGUUUCUUCACAUUCUCUCGAGAAAAAAAAAAAAUGGAAAAAAAAA |
| | | AAACGGAAAAAAAAAAAAGGUAAAAAAAAAAAAUAUAAAAAAAAAAAACAUAAAAAAAAAAAACGAAAAAAAAAAAACGUAAAAAAAAAAA |
| | | ACUCAAAAAAAAAAAAGAUAAAAAAAAAAAACCUAAAAAAAAAAAAUGUAAAAAAAAAAAAGGGAAAAAAAAAAAACGCAAAAAAAAAAAA |
| | | CACAAAAAAAAAAAAUGCAAAAAAAAAAAAUCGAAAAAAAAAAAAUCUAAAAAAAAAAAACGAAAAAAAAAAAACCCAAAAAAAAAAAAGA |
| | | CAAAAAAAAAAAAUAGAAAAAAAAAAAAGUUAAAAAAAAAAAACUGAAAAAAAAAAAAUUUAAAAAAAAAAAAUCUAG |
| 89 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 81 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residue | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | and | CCUGUCCCUGGGCCUGACCCCCAACUUCAAGUCCAACUUCGACCUGGCCGAGGACGCCAAGCUGCAGCUGUCCAAGGACACCUACGACGAC |
| | HiBit | GACCUGGACAACCUGCUGGCCCAGAUCGGCGACCAGUACGCCGACCUGUUCCUGGCCGCCAAGAACCUGUCCGACGCCAUCCUGCUGUCCG |
| | tag- | ACAUCCUGCGGGUGAACACCGAGAUCACCAAGGCCCCCCUGUCCGCCUCCAUGAUCAAGCGGUACGACGAGCACCACCAGGACCUGACCCU |
| | SpyCas9 | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | without | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | Rec3 | GGGAGGACCUGCUGCGGAAGCAGCGGACCUUCGACAACGGCUCCAUCCCCCACCAGAUCCACCUGGGCGAGCUGCACGCCAUCCUGCGGCG |
| | domain | GCAGGAGGACUUCUACCCCUUCCUGAAGGACAACCGGGAGAAGAUCGAGAAGAUCCUGACCUUCCGGAUCCCCUACUACGUGGGCCCCCUG |
| | (mRNA | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | W)- | CCUCCGCCCAGUCCUUCAUCGAGCGGAUGACCGGCGGCUCCUCCGGCCAGGGCGACUCCCUGCACGAGCACAUCGCCAACCUGGCCGGCUC |
| | ORF | CCCCGCCAUCAAGAAGGGCAUCCUGCAGACCGUGAAGGUGGUGGACGAGCUGGUGAAGGUGAUGGGCCGGCACAAGCCCGAGAACAUCGUG |
| | sequence | AUCGAGAUGGCCCGGGAGAACCAGACCACCCAGAAGGGCCAGAAGAACUCCCGGGAGCGGAUGAAGCGGAUCGAGGAGGGCAUCAAGGAGC |
| | | UGGGCUCCCAGAUCCUGAAGGAGCACCCCGUGGAGAACACCCAGCUGCAGAACGAGAAGCUGUACCUGUACUACCUGCAGAACGGCCGGGA |
| | | CAUGUACGUGGACCAGGAGCUGGACAUCAACCGGCUGUCCGACUACGACGUGGACCACAUCGUGCCCCAGUCCUUCCUGAAGGACGACUCC |
| | | AUCGACAACAAGGUGCUGACCCGGUCCGACAAGAACCGGGGCAAGUCCGACAACGUGCCCUCCGAGGAGGUGGUGAAGAAGAUGAAGAACU |
| | | ACUGGCGGCAGCUGCUGAACGCCAAGCUGAUCACCCAGCGGAAGUUCGACAACCUGACCAAGGCCGAGCGGGGCGGCCUGUCCGAGCUGGA |
| | | CAAGGCCGGCUUCAUCAAGCGGCAGCUGGUGGAGACCCGGCAGAUCACCAAGCACGUGGCCCAGAUCCUGGACUCCCGGAUGAACACCAAG |
| | | UACGACGAGAACGACAAGCUGAUCCGGGAGGUGAAGGUGAUCACCCUGAAGUCCAAGCUGGUGUCCGACUUCCGGAAGGACUUCCAGUUCU |
| | | ACAAGGUGCGGGAGAUCAACAACUACCACCACGCCCACGACGCCUACCUGAACGCCGUGGUGGGCACCGCCCUGAUCAAGAAGUACCCCAA |
| | | GCUGGAGUCCGAGUUCGUGUACGGCGACUACAAGGUGUACGACGUGCGGAAGAUGAUCGCCAAGUCCGAGCAGGAGAUCGGCAAGGCCACC |
| | | GCCAAGUACUUCUUCUACUCCAACAUCAUGAACUUCUUCAAGACCGAGAUCACCCUGGCCAACGGCGAGAUCCGGAAGCGGCCCCUGAUCG |
| | | AGACCAACGGCGAGACCGGCGAGAUCGUGUGGGACAAGGGCCGGGACUUCGCCACCGUGCGGAAGGUGCUGUCCAUGCCCCAGGUGAACAU |
| | | CGUGAAGAAGACCGAGGUGCAGACCGGCGGCUUCUCCAAGGAGUCCAUCCUGCCCAAGCGGAACUCCGACAAGCUGAUCGCCCGGAAGAAG |
| | | GACUGGGACCCCAAGAAGUACGGCGGCUUCGACUCCCCCACCGUGGCCUACUCCGUGCUGGUGGUGGCCAAGGUGGAGAAGGGCAAGUCCA |
| | | AGAAGCUGAAGUCCGUGAAGGAGCUGCUGGGCAUCACCAUCAUGGAGCGGUCCUCCUUCGAGAAGAACCCCAUCGACUUCCUGGAGGCCAA |
| | | GGGCUACAAGGAGGUGAAGAAGGACCUGAUCAUCAAGCUGCCCAAGUACUCCCUGUUCGAGCUGGAGAACGGCCGGAAGCGGAUGCUGGCC |
| | | UCCGCCGGCGAGCUGCAGAAGGGCAACGAGCUGGCCCUGCCCUCCAAGUACGUGAACUUCCUGUACCUGGCCUCCCACUACGAGAAGCUGA |
| | | AGGGCUCCCCCGAGGACAACGAGCAGAAGCAGCUGUUCGUGGAGCAGCACAAGCACUACCUGGACGAGAUCAUCGAGCAGAUCUCCGAGUU |
| | | CUCCAAGCGGGUGAUCCUGGCCGACGCCAACCUGGACAAGGUGCUGUCCGCCUACAACAAGCACCGGGACAAGCCCAUCCGGGAGCAGGCC |
| | | GAGAACAUCAUCCACCUGUUCACCCUGACCAACCUGGGCGCCCCCGCCGCCUUCAAGUACUUCGACACCACCAUCGACAAGAAGCGGUACA |
| | | CCUCCACCAAGGAGGUGCUGGACGCCACCCUGAUCCACCAGUCCAUCACCGGCCUGUACGAGACCCGGAUCGACCUGUCCCAGCUGGGCGG |
| | | CGACGGCGCCCCCGAGUCCGCCACCGAGUCCGGCGGCACCUCCACCGAGUCCGAGGGCUCCGCCGGCACCUCCACCGAGUCCGAGGGCUCC |
| | | GCCGGCUCCGCCGGCUCCACCUCCACCGAGGAGGGCACCUCCACCGAGUCCGAGGGCUCCGCCGGCACCUCCACCGAGUCCGAGGGCUCCG |
| | | CCGGCACCUCCGAGUCCGCCACCGAGUCCGGCGGCACCUCCACCGAGUCCGAGGGCUCCUCCUCCGGUGCCGCCUUCAAGCCCAACCCCAU |
| | | CAACUACAUCCUGGGCCUGGACAUCGGCAUCGCCUCCGUGGGCUGGGCCAUGGUGGAGAUCGACGAGGAGGAGAACCCCAUCCGGCUGAUC |
| | | GACCUGGGCGUGCGGGUGUUCGAGCGGGCCGAGGUGCCCAAGACCGGCGACUCCCUGGCCAUGGCCCGGCGGCUGGCCCGGUCCGUGCGGC |
| | | GGCUGACCCGGCGGCGGGCCCACCGGCUGCUGCGGGCCCGGCGGCUGCUGAAGCGGGAGGGCGUGCUGCAGGCCGCCGACUUCGACGAGAA |
| | | CGGCCUGAUCAAGUCCCUGCCCAACACCCCCUGGCAGCUGCGGGCCGCCGCCCUGGACCGGAAGCUGACCCCCCUGGAGUGGUCCGCCGUG |
| | | CUGCUGCACCUGAUCAAGCACCGGGGCUACCUGUCCCAGCGGAAGAACGAGGGCGAGACCGCCGACAAGGAGCUGGGCGCCCUGCUGAAGG |
| | | GCGUGGCCAACAACGCCCACGCCCUGCAGACCGGCGACUUCCGGACCCCCGCCGAGCUGGCCCUGAACAAGUUCGAGAAGGAGUCCGGCCA |
| | | CAUCCGGAACCAGCGGGGCGACUACUCCCACACCUUCUCCCGGAAGGACCUGCAGGCCGAGCUGAUCCUGCUGUUCGAGAAGCAGAAGGAG |
| | | UUCGGCAACCCCCACGUGUCCGGCGGCCUGAAGGAGGGCAUCGAGACCCUGCUGAUGACCCAGCGGCCCGCCCUGUCCGGCGACGCCGUGC |
| | | AGAAGAUGCUGGGCCACUGCACCUUCGAGCCCGCCGAGCCCAAGGCCGCCAAGAACACCUACACCGCCGAGCGGUUCAUCUGGCUGACCAA |
| | | GCUGAACAACCUGCGGAUCCUGGAGCAGGGCUCCGAGCGGCCCCUGACCGACACCGAGCGGGCCACCCUGAUGGACGAGCCCUACCGGAAG |
| | | UCCAAGCUGACCUACGCCCAGGCCCGGAAGCUGCUGGGCCUGGAGGACACCGCCUUCUUCAAGGGCCUGCGGUACGGCAAGGACAACGCCG |
| | | AGGCCUCCACCCUGAUGGAGAUGAAGGCCUACCACGCCAUCUCCCGGGCCCUGGAGAAGGAGGGCCUGAAGGACAAGAAGUCCCCCCUGAA |
| | | CCUGUCCUCCGAGCUGCAGGACGAGAUCGGCACCGCCUUCUCCCUGUUCAAGACCGACGAGGACAUCACCGGCCGGCUGAAGGACCGGGUG |
| | | CAGCCCGAGAUCCUGGAGGCCCUGCUGAAGCACAUCUCCUUCGACAAGUUCGUGCAGAUCUCCCUGAAGGCCCUGCGGCGGAUCGUGCCCC |
| | | UGAUGGAGCAGGGCAAGCGGUACGACGAGGCCUGCGCCGAGAUCUACGGCGACCACUACGGCAAGAAGAACACCGAGGAGAAGAUCUACCU |
| | | GCCCCCCAUCCCCGCCGACGAGAUCCGGAACCCCGUGGUGCUGCGGGCCCUGUCCCAGGCCCGGAAGGUGAUCAACGGCGUGGUGCGGCGG |
| | | UACGGCUCCCCCGCCCGGAUCCACAUCGAGACCGCCCGGGAGGUGGGCAAGUCCUUCAAGGACCGGAAGGAGAUCGAGAAGCGGCAGGAGG |
| | | AGAACCGGAAGGACCGGGAGAAGGCCGCCGCCAAGUUCCGGGAGUACUUCCCCAACUUCGUGGGCGAGCCCAAGUCCAAGGACAUCCUGAA |
| | | GCUGCGGCUGUACGAGCAGCAGCACGGCAAGUGCCUGUACUCCGGCAAGGAGAUCAACCUGGUGCGGCUGAACGAGAAGGGCUACGUGGAG |
| | | AUCGACCACGCCCUGCCCUUCUCCCGGACCUGGGACGACUCCUUCAACAACAAGGUGCUGGUGCUGGGCUCCGAGAACCAGAACAAGGGCA |
| | | ACCAGACCCCCUACGAGUACUUCAACGGCAAGGACAACUCCCGGGAGUGGCAGGAGUUCAAGGCCCGGGUGGAGACCUCCCGGUUCCCCCG |
| | | GUCCAAGAAGCAGCGGAUCCUGCUGCAGAAGUUCGACGAGGACGGCUUCAAGGAGUGCAACCUGAACGACACCCGGUACGUGAACCGCUUC |
| | | CUGUGCCAGUUCGUGGCCGACCACAUCCUGCUGACCGGCAAGGGCAAGCGGCGGGUGUUCGCCUCCAACGGCCAGAUCACCAACCUGCUGC |
| | | GGGGCUUCUGGGGCCUGCGGAAGGUGCGGGCCGAGAACGACCGGCACCACGCCCUGGACGCCGUGGUGGUGGCCUGCUCCACCGUGGCCAU |
| | | GCAGCAGAAGAUCACCCGGUUCGUGCGGUACAAGGAGAUGAACGCCUUCGACGGCAAGACCAUCGACAAGGAGACCGGCAAGGUGCUGCAC |
| | | CAGAAGACCCACUUCCCCCAGCCCUGGGAGUUCUUCGCCCAGGAGGUGAUGAUCCGGGUGUUCGGCAAGCCCGACGGCAAGCCCGAGUUCG |
| | | AGGAGGCCGACACCCCCGAGAAGCUGCGGACCCUGCUGGCCGAGAAGCUGUCCUCCCGGCCCGAGGCCGUGCACGAGUACGUGACCCCCCU |
| | | GUUCGUGUCCCGGGCCCCCAACCGGAAGAUGUCCGGCGCCCACAAGGACACCCUGCGGUCCGCCAAGCGGUUCGUGAAGCACAACGAGAAG |
| | | AUCUCCGUGAAGCGGGUGUGGCUGACCGAGAUCAAGCUGGCCGACCUGGAGAACAUGGUGAACUACAAGAACGGCCGGGAGAUCGAGCUGU |
| | | ACGAGGCCCUGAAGGCCCGGCUGGAGGCCUACGGCGGCAACGCCAAGCAGGCCUUCGACCCCAAGGACAACCCCUUCUACAAGAAGGGCGG |
| | | CCAGCUGGUGAAGGCCGUGCGGGUGGAGAAGACCCAGGAGUCCGGCGUGCUGCUGAACAAGAAGAACGCCUACACCAUCGCCGACAACGGC |
| | | GACAUGGUGCGGGUGGACGUGUUCUGCAAGGUGGACAAGAAGGGCAAGAACCAGUACUUCAUCGUGCCCAUCUACGCCUGGCAGGUGGCCG |
| | | AGAACAUCCUGCCCGACAUCGACUGCAAGGGCUACCGGAUCGACGACUCCUACACCUUCUGCUUCUCCCUGCACAAGUACGACCUGAUCGC |
| | | CUUCCAGAAGGACGAGAAGUCCAAGGUGGAGUUCGCCUACUACAUCAACUGCGACUCCUCCAACGGCCGGUUCUACCUGGCCUGGCACGAC |
| | | AAGGGCUCCAAGGAGCAGCAGUUCCGGAUCUCCACCCAGAACCUGGUGCUGAUCCAGAAGUACCAGGUGAACGAGCUGGGCAAGGAGAUCC |
| | | GGCCCUGCCGGCUGAAGAAGCGGCCCCCCGUGCGGUCCGGAAAGCGGACCGCCGACGGCUCCGGAGGAGGAAGCCCGAAGAAGAAGAGAAA |
| | | GGUCAGCGAAAGCGCAACACCGGAAAGCGUCAGCGGAUGGAGACUGUUCAAGAAGAUCAGCUAG |
| 90 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 81 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTGGSSGQGDSLHEHIANLAGSPAIKKGILQTVKVVDELVKVMGRHKPENIV |
| | residue | IEMARENQTTQKGQKNSRERMKRIEEGIKELGSQILKEHPVENTQLQNEKLYLYYLQNGRDMYVDQELDINRLSDYDVDHIVPQSFLKDDS |
| | linker | IDNKVLTRSDKNRGKSDNVPSEEVVKKMKNYWRQLLNAKLITQRKFDNLTKAERGGLSELDKAGFIKRQLVETRQITKHVAQILDSRMNTK |
| | and | YDENDKLIREVKVITLKSKLVSDFRKDFQFYKVREINNYHHAHDAYLNAVVGTALIKKYPKLESEFVYGDYKVYDVRKMIAKSEQEIGKAT |
| | HiBit | AKYFFYSNIMNFFKTEITLANGEIRKRPLIETNGETGEIVWDKGRDFATVRKVLSMPQVNIVKKTEVQTGGFSKESILPKRNSDKLIARKK |
| | tag- | DWDPKKYGGFDSPTVAYSVLVVAKVEKGKSKKLKSVKELLGITIMERSSFEKNPIDFLEAKGYKEVKKDLIIKLPKYSLFELENGRKRMLA |
| | SpyCas9 | SAGELQKGNELALPSKYVNFLYLASHYEKLKGSPEDNEQKQLFVEQHKHYLDEIIEQISEFSKRVILADANLDKVLSAYNKHRDKPIREQA |
| | without | ENIIHLFTLTNLGAPAAFKYFDTTIDKKRYTSTKEVLDATLIHQSITGLYETRIDLSQLGGDGAPESATESGGTSTESEGSAGTSTESEGS |
| | Rec3 | AGSAGSTSTEEGTSTESEGSAGTSTESEGSAGTSESATESGGTSTESEGSSSGAAFKPNPINYILGLDIGIASVGWAMVEIDEEENPIRLI |
| | domain | DLGVRVFERAEVPKTGDSLAMARRLARSVRRLTRRRAHRLLRARRLLKREGVLQAADFDENGLIKSLPNTPWQLRAAALDRKLTPLEWSAV |
| | (mRNA | LLHLIKHRGYLSQRKNEGETADKELGALLKGVANNAHALQTGDFRTPAELALNKFEKESGHIRNQRGDYSHTFSRKDLQAELILLFEKQKE |
| | W)- | FGNPHVSGGLKEGIETLLMTQRPALSGDAVQKMLGHCTFEPAEPKAAKNTYTAERFIWLTKLNNLRILEQGSERPLTDTERATLMDEPYRK |
| | amino | SKLTYAQARKLLGLEDTAFFKGLRYGKDNAEASTLMEMKAYHAISRALEKEGLKDKKSPLNLSSELQDEIGTAFSLFKTDEDITGRLKDRV |
| | acid | QPEILEALLKHISFDKFVQISLKALRRIVPLMEQGKRYDEACAEIYGDHYGKKNTEEKIYLPPIPADEIRNPVVLRALSQARKVINGVVRR |
| | sequence | YGSPARIHIETAREVGKSFKDRKEIEKRQEENRKDREKAAAKFREYFPNFVGEPKSKDILKLRLYEQQHGKCLYSGKEINLVRLNEKGYVE |
| | | IDHALPFSRTWDDSFNNKVLVLGSENQNKGNQTPYEYFNGKDNSREWQEFKARVETSRFPRSKKQRILLQKFDEDGFKECNLNDTRYVNRF |
| | | LCQFVADHILLTGKGKRRVFASNGQITNLLRGFWGLRKVRAENDRHHALDAVVVACSTVAMQQKITRFVRYKEMNAFDGKTIDKETGKVLH |
| | | QKTHFPQPWEFFAQEVMIRVFGKPDGKPEFEEADTPEKLRTLLAEKLSSRPEAVHEYVTPLFVSRAPNRKMSGAHKDTLRSAKRFVKHNEK |
| | | ISVKRVWLTEIKLADLENMVNYKNGREIELYEALKARLEAYGGNAKQAFDPKDNPFYKKGGQLVKAVRVEKTQESGVLLNKKNAYTIADNG |
| | | DMVRVDVFCKVDKKGKNQYFIVPIYAWQVAENILPDIDCKGYRIDDSYTFCFSLHKYDLIAFQKDEKSKVEFAYYINCDSSNGRFYLAWHD |
| | | KGSKEQQFRISTQNLVLIQKYQVNELGKEIRPCRLKKRPPVRSGKRTADGSGGGSPKKKRKVSESATPESVSGWRLFKKIS* |
| 91 | Cas9- | AUGGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | fusion- | CACCCGGCUGAAGCGGACCGCCCGGCGGCGGUACACCCGGCGGAAGAACCGGAUCUGCUACCUGCAGGAGAUCUUCUCCAACGAGAUGGCC |
| | 81 | AAGGUGGACGACUCCUUCUUCCACCGGCUGGAGGAGUCCUUCCUGGUGGAGGAGGACAAGAAGCACGAGCGGCACCCCAUCUUCGGCAACA |
| | amino | UCGUGGACGAGGUGGCCUACCACGAGAAGUACCCCACCAUCUACCACCUGCGGAAGAAGCUGGUGGACUCCACCGACAAGGCCGACCUGCG |
| | acid | GCUGAUCUACCUGGCCCUGGCCCACAUGAUCAAGUUCCGGGGCCACUUCCUGAUCGAGGGCGACCUGAACCCCGACAACUCCGACGUGGAC |
| | residual | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | linker | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | with | CCUGUCCCUGGGCCUGACCCCCAACUUCAAGUCCAACUUCGACCUGGCCGAGGACGCCAAGCUGCAGCUGUCCAAGGACACCUACGACGAC |
| | SpyCas9 | GACCUGGACAACCUGCUGGCCCAGAUCGGCGACCAGUACGCCGACCUGUUCCUGGCCGCCAAGAACCUGUCCGACGCCAUCCUGCUGUCCG |
| | without | ACAUCCUGCGGGUGAACACCGAGAUCACCAAGGCCCCCCUGUCCGCCUCCAUGAUCAAGCGGUACGACGAGCACCACCAGGACCUGACCCU |
| | Rec3 | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | domain | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | (mRNA | GGGAGGACCUGCUGCGGAAGCAGCGGACCUUCGACAACGGCUCCAUCCCCCACCAGAUCCACCUGGGCGAGCUGCACGCCAUCCUGCGGCG |
| | W)- | GCAGGAGGACUUCUACCCCUUCCUGAAGGACAACCGGGAGAAGAUCGAGAAGAUCCUGACCUUCCGGAUCCCCUACUACGUGGGCCCCCUG |
| | mRNA | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | sequence | CCUCCGCCCAGUCCUUCAUCGAGCGGAUGACCGGCGGCUCCUCCGGCCAGGGCGACUCCCUGCACGAGCACAUCGCCAACCUGGCCGGCUC |
| | | CCCCGCCAUCAAGAAGGGCAUCCUGCAGACCGUGAAGGUGGUGGACGAGCUGGUGAAGGUGAUGGGCCGGCACAAGCCCGAGAACAUCGUG |
| | | AUCGAGAUGGCCCGGGAGAACCAGACCACCCAGAAGGGCCAGAAGAACUCCCGGGAGCGGAUGAAGCGGAUCGAGGAGGGCAUCAAGGAGC |
| | | UGGGCUCCCAGAUCCUGAAGGAGCACCCCGUGGAGAACACCCAGCUGCAGAACGAGAAGCUGUACCUGUACUACCUGCAGAACGGCCGGGA |
| | | CAUGUACGUGGACCAGGAGCUGGACAUCAACCGGCUGUCCGACUACGACGUGGACCACAUCGUGCCCCAGUCCUUCCUGAAGGACGACUCC |
| | | AUCGACAACAAGGUGCUGACCCGGUCCGACAAGAACCGGGGCAAGUCCGACAACGUGCCCUCCGAGGAGGUGGUGAAGAAGAUGAAGAACU |
| | | ACUGGCGGCAGCUGCUGAACGCCAAGCUGAUCACCCAGCGGAAGUUCGACAACCUGACCAAGGCCGAGCGGGGCGGCCUGUCCGAGCUGGA |
| | | CAAGGCCGGCUUCAUCAAGCGGCAGCUGGUGGAGACCCGGCAGAUCACCAAGCACGUGGCCCAGAUCCUGGACUCCCGGAUGAACACCAAG |
| | | UACGACGAGAACGACAAGCUGAUCCGGGAGGUGAAGGUGAUCACCCUGAAGUCCAAGCUGGUGUCCGACUUCCGGAAGGACUUCCAGUUCU |
| | | ACAAGGUGCGGGAGAUCAACAACUACCACCACGCCCACGACGCCUACCUGAACGCCGUGGUGGGCACCGCCCUGAUCAAGAAGUACCCCAA |
| | | GCUGGAGUCCGAGUUCGUGUACGGCGACUACAAGGUGUACGACGUGCGGAAGAUGAUCGCCAAGUCCGAGCAGGAGAUCGGCAAGGCCACC |
| | | GCCAAGUACUUCUUCUACUCCAACAUCAUGAACUUCUUCAAGACCGAGAUCACCCUGGCCAACGGCGAGAUCCGGAAGCGGCCCCUGAUCG |
| | | AGACCAACGGCGAGACCGGCGAGAUCGUGUGGGACAAGGGCCGGGACUUCGCCACCGUGCGGAAGGUGCUGUCCAUGCCCCAGGUGAACAU |
| | | CGUGAAGAAGACCGAGGUGCAGACCGGCGGCUUCUCCAAGGAGUCCAUCCUGCCCAAGCGGAACUCCGACAAGCUGAUCGCCCGGAAGAAG |
| | | GACUGGGACCCCAAGAAGUACGGCGGCUUCGACUCCCCCACCGUGGCCUACUCCGUGCUGGUGGUGGCCAAGGUGGAGAAGGGCAAGUCCA |
| | | AGAAGCUGAAGUCCGUGAAGGAGCUGCUGGGCAUCACCAUCAUGGAGCGGUCCUCCUUCGAGAAGAACCCCAUCGACUUCCUGGAGGCCAA |
| | | GGGCUACAAGGAGGUGAAGAAGGACCUGAUCAUCAAGCUGCCCAAGUACUCCCUGUUCGAGCUGGAGAACGGCCGGAAGCGGAUGCUGGCC |
| | | UCCGCCGGCGAGCUGCAGAAGGGCAACGAGCUGGCCCUGCCCUCCAAGUACGUGAACUUCCUGUACCUGGCCUCCCACUACGAGAAGCUGA |
| | | AGGGCUCCCCCGAGGACAACGAGCAGAAGCAGCUGUUCGUGGAGCAGCACAAGCACUACCUGGACGAGAUCAUCGAGCAGAUCUCCGAGUU |
| | | CUCCAAGCGGGUGAUCCUGGCCGACGCCAACCUGGACAAGGUGCUGUCCGCCUACAACAAGCACCGGGACAAGCCCAUCCGGGAGCAGGCC |
| | | GAGAACAUCAUCCACCUGUUCACCCUGACCAACCUGGGCGCCCCCGCCGCCUUCAAGUACUUCGACACCACCAUCGACAAGAAGCGGUACA |
| | | CCUCCACCAAGGAGGUGCUGGACGCCACCCUGAUCCACCAGUCCAUCACCGGCCUGUACGAGACCCGGAUCGACCUGUCCCAGCUGGGCGG |
| | | CGACGGCGCCCCCGAGUCCGCCACCGAGUCCGGCGGCACCUCCACCGAGUCCGAGGGCUCCGCCGGCACCUCCACCGAGUCCGAGGGCUCC |
| | | GCCGGCUCCGCCGGCUCCACCUCCACCGAGGAGGGCACCUCCACCGAGUCCGAGGGCUCCGCCGGCACCUCCACCGAGUCCGAGGGCUCCG |
| | | CCGGCACCUCCGAGUCCGCCACCGAGUCCGGCGGCACCUCCACCGAGUCCGAGGGCUCCUCCUCCGGUGCCGCCUUCAAGCCCAACCCCAU |
| | | CAACUACAUCCUGGGCCUGGACAUCGGCAUCGCCUCCGUGGGCUGGGCCAUGGUGGAGAUCGACGAGGAGGAGAACCCCAUCCGGCUGAUC |
| | | GACCUGGGCGUGCGGGUGUUCGAGCGGGCCGAGGUGCCCAAGACCGGCGACUCCCUGGCCAUGGCCCGGCGGCUGGCCCGGUCCGUGCGGC |
| | | GGCUGACCCGGCGGCGGGCCCACCGGCUGCUGCGGGCCCGGCGGCUGCUGAAGCGGGAGGGCGUGCUGCAGGCCGCCGACUUCGACGAGAA |
| | | CGGCCUGAUCAAGUCCCUGCCCAACACCCCCUGGCAGCUGCGGGCCGCCGCCCUGGACCGGAAGCUGACCCCCCUGGAGUGGUCCGCCGUG |
| | | CUGCUGCACCUGAUCAAGCACCGGGGCUACCUGUCCCAGCGGAAGAACGAGGGCGAGACCGCCGACAAGGAGCUGGGCGCCCUGCUGAAGG |
| | | GCGUGGCCAACAACGCCCACGCCCUGCAGACCGGCGACUUCCGGACCCCCGCCGAGCUGGCCCUGAACAAGUUCGAGAAGGAGUCCGGCCA |
| | | CAUCCGGAACCAGCGGGGCGACUACUCCCACACCUUCUCCCGGAAGGACCUGCAGGCCGAGCUGAUCCUGCUGUUCGAGAAGCAGAAGGAG |
| | | UUCGGCAACCCCCACGUGUCCGGCGGCCUGAAGGAGGGCAUCGAGACCCUGCUGAUGACCCAGCGGCCCGCCCUGUCCGGCGACGCCGUGC |
| | | AGAAGAUGCUGGGCCACUGCACCUUCGAGCCCGCCGAGCCCAAGGCCGCCAAGAACACCUACACCGCCGAGCGGUUCAUCUGGCUGACCAA |
| | | GCUGAACAACCUGCGGAUCCUGGAGCAGGGCUCCGAGCGGCCCCUGACCGACACCGAGCGGGCCACCCUGAUGGACGAGCCCUACCGGAAG |
| | | UCCAAGCUGACCUACGCCCAGGCCCGGAAGCUGCUGGGCCUGGAGGACACCGCCUUCUUCAAGGGCCUGCGGUACGGCAAGGACAACGCCG |
| | | AGGCCUCCACCCUGAUGGAGAUGAAGGCCUACCACGCCAUCUCCCGGGCCCUGGAGAAGGAGGGCCUGAAGGACAAGAAGUCCCCCCUGAA |
| | | CCUGUCCUCCGAGCUGCAGGACGAGAUCGGCACCGCCUUCUCCCUGUUCAAGACCGACGAGGACAUCACCGGCCGGCUGAAGGACCGGGUG |
| | | CAGCCCGAGAUCCUGGAGGCCCUGCUGAAGCACAUCUCCUUCGACAAGUUCGUGCAGAUCUCCCUGAAGGCCCUGCGGCGGAUCGUGCCCC |
| | | UGAUGGAGCAGGGCAAGCGGUACGACGAGGCCUGCGCCGAGAUCUACGGCGACCACUACGGCAAGAAGAACACCGAGGAGAAGAUCUACCU |
| | | GCCCCCCAUCCCCGCCGACGAGAUCCGGAACCCCGUGGUGCUGCGGGCCCUGUCCCAGGCCCGGAAGGUGAUCAACGGCGUGGUGCGGCGG |
| | | UACGGCUCCCCCGCCCGGAUCCACAUCGAGACCGCCCGGGAGGUGGGCAAGUCCUUCAAGGACCGGAAGGAGAUCGAGAAGCGGCAGGAGG |
| | | AGAACCGGAAGGACCGGGAGAAGGCCGCCGCCAAGUUCCGGGAGUACUUCCCCAACUUCGUGGGCGAGCCCAAGUCCAAGGACAUCCUGAA |
| | | GCUGCGGCUGUACGAGCAGCAGCACGGCAAGUGCCUGUACUCCGGCAAGGAGAUCAACCUGGUGCGGCUGAACGAGAAGGGCUACGUGGAG |
| | | AUCGACCACGCCCUGCCCUUCUCCCGGACCUGGGACGACUCCUUCAACAACAAGGUGCUGGUGCUGGGCUCCGAGAACCAGAACAAGGGCA |
| | | ACCAGACCCCCUACGAGUACUUCAACGGCAAGGACAACUCCCGGGAGUGGCAGGAGUUCAAGGCCCGGGUGGAGACCUCCCGGUUCCCCCG |
| | | GUCCAAGAAGCAGCGGAUCCUGCUGCAGAAGUUCGACGAGGACGGCUUCAAGGAGUGCAACCUGAACGACACCCGGUACGUGAACCGCUUC |
| | | CUGUGCCAGUUCGUGGCCGACCACAUCCUGCUGACCGGCAAGGGCAAGCGGCGGGUGUUCGCCUCCAACGGCCAGAUCACCAACCUGCUGC |
| | | GGGGCUUCUGGGGCCUGCGGAAGGUGCGGGCCGAGAACGACCGGCACCACGCCCUGGACGCCGUGGUGGUGGCCUGCUCCACCGUGGCCAU |
| | | GCAGCAGAAGAUCACCCGGUUCGUGCGGUACAAGGAGAUGAACGCCUUCGACGGCAAGACCAUCGACAAGGAGACCGGCAAGGUGCUGCAC |
| | | CAGAAGACCCACUUCCCCCAGCCCUGGGAGUUCUUCGCCCAGGAGGUGAUGAUCCGGGUGUUCGGCAAGCCCGACGGCAAGCCCGAGUUCG |
| | | AGGAGGCCGACACCCCCGAGAAGCUGCGGACCCUGCUGGCCGAGAAGCUGUCCUCCCGGCCCGAGGCCGUGCACGAGUACGUGACCCCCCU |
| | | GUUCGUGUCCCGGGCCCCCAACCGGAAGAUGUCCGGCGCCCACAAGGACACCCUGCGGUCCGCCAAGCGGUUCGUGAAGCACAACGAGAAG |
| | | AUCUCCGUGAAGCGGGUGUGGCUGACCGAGAUCAAGCUGGCCGACCUGGAGAACAUGGUGAACUACAAGAACGGCCGGGAGAUCGAGCUGU |
| | | ACGAGGCCCUGAAGGCCCGGCUGGAGGCCUACGGCGGCAACGCCAAGCAGGCCUUCGACCCCAAGGACAACCCCUUCUACAAGAAGGGCGG |
| | | CCAGCUGGUGAAGGCCGUGCGGGUGGAGAAGACCCAGGAGUCCGGCGUGCUGCUGAACAAGAAGAACGCCUACACCAUCGCCGACAACGGC |
| | | GACAUGGUGCGGGUGGACGUGUUCUGCAAGGUGGACAAGAAGGGCAAGAACCAGUACUUCAUCGUGCCCAUCUACGCCUGGCAGGUGGCCG |
| | | AGAACAUCCUGCCCGACAUCGACUGCAAGGGCUACCGGAUCGACGACUCCUACACCUUCUGCUUCUCCCUGCACAAGUACGACCUGAUCGC |
| | | CUUCCAGAAGGACGAGAAGUCCAAGGUGGAGUUCGCCUACUACAUCAACUGCGACUCCUCCAACGGCCGGUUCUACCUGGCCUGGCACGAC |
| | | AAGGGCUCCAAGGAGCAGCAGUUCCGGAUCUCCACCCAGAACCUGGUGCUGAUCCAGAAGUACCAGGUGAACGAGCUGGGCAAGGAGAUCC |
| | | GGCCCUGCCGGCUGAAGAAGCGGCCCCCCGUGCGGUCCGGAAAGCGGACCGCCGACGGCUCCGGAGGAGGAAGCCCGAAGAAGAAGAGAAA |
| | | GGUCUAG |
| 92 | Cas9- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | Cas9 | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | fusion- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | 81 | DLDNLLAQIGDQYADLFLAAKNLSDAILLSDILRVNTEITKAPLSASMIKRYDEHHQDLTLLKALVRQQLPEKYKEIFFDQSKNGYAGYID |
| | amino | GGASQEEFYKFIKPILEKMDGTEELLVKLNREDLLRKQRTFDNGSIPHQIHLGELHAILRRQEDFYPFLKDNREKIEKILTFRIPYYVGPL |
| | acid | ARGNSRFAWMTRKSEETITPWNFEEVVDKGASAQSFIERMTGGSSGQGDSLHEHIANLAGSPAIKKGILQTVKVVDELVKVMGRHKPENIV |
| | residual | IEMARENQTTQKGQKNSRERMKRIEEGIKELGSQILKEHPVENTQLQNEKLYLYYLQNGRDMYVDQELDINRLSDYDVDHIVPQSFLKDDS |
| | linker | IDNKVLTRSDKNRGKSDNVPSEEVVKKMKNYWRQLLNAKLITQRKFDNLTKAERGGLSELDKAGFIKRQLVETRQITKHVAQILDSRMNTK |
| | with | YDENDKLIREVKVITLKSKLVSDFRKDFQFYKVREINNYHHAHDAYLNAVVGTALIKKYPKLESEFVYGDYKVYDVRKMIAKSEQEIGKAT |
| | SpyCas9 | AKYFFYSNIMNFFKTEITLANGEIRKRPLIETNGETGEIVWDKGRDFATVRKVLSMPQVNIVKKTEVQTGGFSKESILPKRNSDKLIARKK |
| | without | DWDPKKYGGFDSPTVAYSVLVVAKVEKGKSKKLKSVKELLGITIMERSSFEKNPIDFLEAKGYKEVKKDLIIKLPKYSLFELENGRKRMLA |
| | Rec3 | SAGELQKGNELALPSKYVNFLYLASHYEKLKGSPEDNEQKQLFVEQHKHYLDEIIEQISEFSKRVILADANLDKVLSAYNKHRDKPIREQA |
| | domain | ENIIHLFTLTNLGAPAAFKYFDTTIDKKRYTSTKEVLDATLIHQSITGLYETRIDLSQLGGDGAPESATESGGTSTESEGSAGTSTESEGS |
| | (mRNA- | AGSAGSTSTEEGTSTESEGSAGTSTESEGSAGTSESATESGGTSTESEGSSSGAAFKPNPINYILGLDIGIASVGWAMVEIDEEENPIRLI |
| | W) | DLGVRVFERAEVPKTGDSLAMARRLARSVRRLTRRRAHRLLRARRLLKREGVLQAADFDENGLIKSLPNTPWQLRAAALDRKLTPLEWSAV |
| | amino | LLHLIKHRGYLSQRKNEGETADKELGALLKGVANNAHALQTGDFRTPAELALNKFEKESGHIRNQRGDYSHTFSRKDLQAELILLFEKQKE |
| | acid | FGNPHVSGGLKEGIETLLMTQRPALSGDAVQKMLGHCTFEPAEPKAAKNTYTAERFIWLTKLNNLRILEQGSERPLTDTERATLMDEPYRK |
| | sequence | SKLTYAQARKLLGLEDTAFFKGLRYGKDNAEASTLMEMKAYHAISRALEKEGLKDKKSPLNLSSELQDEIGTAFSLFKTDEDITGRLKDRV |
| | | QPEILEALLKHISFDKFVQISLKALRRIVPLMEQGKRYDEACAEIYGDHYGKKNTEEKIYLPPIPADEIRNPVVLRALSQARKVINGVVRR |
| | | YGSPARIHIETAREVGKSFKDRKEIEKRQEENRKDREKAAAKFREYFPNFVGEPKSKDILKLRLYEQQHGKCLYSGKEINLVRLNEKGYVE |
| | | IDHALPFSRTWDDSFNNKVLVLGSENQNKGNQTPYEYFNGKDNSREWQEFKARVETSRFPRSKKQRILLQKFDEDGFKECNLNDTRYVNRF |
| | | LCQFVADHILLTGKGKRRVFASNGQITNLLRGFWGLRKVRAENDRHHALDAVVVACSTVAMQQKITRFVRYKEMNAFDGKTIDKETGKVLH |
| | | QKTHFPQPWEFFAQEVMIRVFGKPDGKPEFEEADTPEKLRTLLAEKLSSRPEAVHEYVTPLFVSRAPNRKMSGAHKDTLRSAKRFVKHNEK |
| | | ISVKRVWLTEIKLADLENMVNYKNGREIELYEALKARLEAYGGNAKQAFDPKDNPFYKKGGQLVKAVRVEKTQESGVLLNKKNAYTIADNG |
| | | DMVRVDVFCKVDKKGKNQYFIVPIYAWQVAENILPDIDCKGYRIDDSYTFCFSLHKYDLIAFQKDEKSKVEFAYYINCDSSNGRFYLAWHD |
| | | KGSKEQQFRISTQNLVLIQKYQVNELGKEIRPCRLKKRPPVRSGKRTADGSGGGSPKKKRKV* |
| 93 | SpyCas9 | GGGUCCCGCAGUCGGCGUCCAGCGGCUCUGCUUGUUCGUGUGUGUGUCGUUGCAGGCCUUAUUCGGAUCCGCCACCAUGGACAAGAAGUAC |
| | (mRNA | AGCAUCGGACUGGACAUCGGAACAAACAGCGUCGGAUGGGCAGUCAUCACAGACGAAUACAAGGUCCCGAGCAAGAAGUUCAAGGUCCUGG |
| | X)- | GAAACACAGACAGACACAGCAUCAAGAAGAACCUGAUCGGAGCACUGCUGUUCGACAGCGGAGAAACAGCAGAAGCAACAAGACUGAAGAG |
| | mRNA | AACAGCAAGAAGAAGAUACACAAGAAGAAAGAACAGAAUCUGCUACCUGCAGGAAAUCUUCAGCAACGAAAUGGCAAAGGUCGACGACAGC |
| | sequence | UUCUUCCACAGACUGGAAGAAAGCUUCCUGGUCGAAGAAGACAAGAAGCACGAAAGACACCCGAUCUUCGGAAACAUCGUCGACGAAGUCG |
| | | CAUACCACGAAAAGUACCCGACAAUCUACCACCUGAGAAAGAAGCUGGUCGACAGCACAGACAAGGCAGACCUGAGACUGAUCUACCUGGC |
| | | ACUGGCACACAUGAUCAAGUUCAGAGGACACUUCCUGAUCGAAGGAGACCUGAACCCGGACAACAGCGACGUCGACAAGCUGUUCAUCCAG |
| | | CUGGUCCAGACAUACAACCAGCUGUUCGAAGAAAACCCGAUCAACGCAAGCGGAGUCGACGCAAAGGCAAUCCUGAGCGCAAGACUGAGCA |
| | | AGAGCAGAAGACUGGAAAACCUGAUCGCACAGCUGCCGGGAGAAAAGAAGAACGGACUGUUCGGAAACCUGAUCGCACUGAGCCUGGGACU |
| | | GACACCGAACUUCAAGAGCAACUUCGACCUGGCAGAAGACGCAAAGCUGCAGCUGAGCAAGGACACAUACGACGACGACCUGGACAACCUG |
| | | CUGGCACAGAUCGGAGACCAGUACGCAGACCUGUUCCUGGCAGCAAAGAACCUGAGCGACGCAAUCCUGCUGAGCGACAUCCUGAGAGUCA |
| | | ACACAGAAAUCACAAAGGCACCGCUGAGCGCAAGCAUGAUCAAGAGAUACGACGAACACCACCAGGACCUGACACUGCUGAAGGCACUGGU |
| | | CAGACAGCAGCUGCCGGAAAAGUACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAGCUGAACAGAGAAGACCUGCUGA |
| | | GAAAGCAGAGAACAUUCGACAACGGAAGCAUCCCGCACCAGAUCCACCUGGGAGAACUGCACGCAAUCCUGAGAAGACAGGAAGACUUCUA |
| | | CCCGUUCCUGAAGGACAACAGAGAAAAGAUCGAAAAGAUCCUGACAUUCAGAAUCCCGUACUACGUCGGACCGCUGGCAAGAGGAAACAGC |
| | | AGAUUCGCAUGGAUGACAAGAAAGAGCGAAGAAACAAUCACACCGUGGAACUUCGAAGAAGUCGUCGACAAGGGAGCAAGCGCACAGAGCU |
| | | UCAUCGAAAGAAUGACAAACUUCGACAAGAACCUGCCGAACGAAAAGGUCCUGCCGAAGCACAGCCUGCUGUACGAAUACUUCACAGUCUA |
| | | CAACGAACUGACAAAGGUCAAGUACGUCACAGAAGGAAUGAGAAAGCCGGCAUUCCUGAGCGGAGAACAGAAGAAGGCAAUCGUCGACCUG |
| | | CUGUUCAAGACAAACAGAAAGGUCACAGUCAAGCAGCUGAAGGAAGACUACUUCAAGAAGAUCGAAUGCUUCGACAGCGUCGAAAUCAGCG |
| | | GAGUCGAAGACAGAUUCAACGCAAGCCUGGGAACAUACCACGACCUGCUGAAGAUCAUCAAGGACAAGGACUUCCUGGACAACGAAGAAAA |
| | | CGAAGACAUCCUGGAAGACAUCGUCCUGACACUGACACUGUUCGAAGACAGAGAAAUGAUCGAAGAAAGACUGAAGACAUACGCACACCUG |
| | | UUCGACGACAAGGUCAUGAAGCAGCUGAAGAGAAGAAGAUACACAGGAUGGGGAAGACUGAGCAGAAAGCUGAUCAACGGAAUCAGAGACA |
| | | AGCAGAGCGGAAAGACAAUCCUGGACUUCCUGAAGAGCGACGGAUUCGCAAACAGAAACUUCAUGCAGCUGAUCCACGACGACAGCCUGAC |
| | | AUUCAAGGAAGACAUCCAGAAGGCACAGGUCAGCGGACAGGGAGACAGCCUGCACGAACACAUCGCAAACCUGGCAGGAAGCCCGGCAAUC |
| | | AAGAAGGGAAUCCUGCAGACAGUCAAGGUCGUCGACGAACUGGUCAAGGUCAUGGGAAGACACAAGCCGGAAAACAUCGUCAUCGAAAUGG |
| | | CAAGAGAAAACCAGACAACACAGAAGGGACAGAAGAACAGCAGAGAAAGAAUGAAGAGAAUCGAAGAAGGAAUCAAGGAACUGGGAAGCCA |
| | | GAUCCUGAAGGAACACCCGGUCGAAAACACACAGCUGCAGAACGAAAAGCUGUACCUGUACUACCUGCAGAACGGAAGAGACAUGUACGUC |
| | | GACCAGGAACUGGACAUCAACAGACUGAGCGACUACGACGUCGACCACAUCGUCCCGCAGAGCUUCCUGAAGGACGACAGCAUCGACAACA |
| | | AGGUCCUGACAAGAAGCGACAAGAACAGAGGAAAGAGCGACAACGUCCCGAGCGAAGAAGUCGUCAAGAAGAUGAAGAACUACUGGAGACA |
| | | GCUGCUGAACGCAAAGCUGAUCACACAGAGAAAGUUCGACAACCUGACAAAGGCAGAGAGAGGAGGACUGAGCGAACUGGACAAGGCAGGA |
| | | UUCAUCAAGAGACAGCUGGUCGAAACAAGACAGAUCACAAAGCACGUCGCACAGAUCCUGGACAGCAGAAUGAACACAAAGUACGACGAAA |
| | | ACGACAAGCUGAUCAGAGAAGUCAAGGUCAUCACACUGAAGAGCAAGCUGGUCAGCGACUUCAGAAAGGACUUCCAGUUCUACAAGGUCAG |
| | | AGAAAUCAACAACUACCACCACGCACACGACGCAUACCUGAACGCAGUCGUCGGAACAGCACUGAUCAAGAAGUACCCGAAGCUGGAAAGC |
| | | GAAUUCGUCUACGGAGACUACAAGGUCUACGACGUCAGAAAGAUGAUCGCAAAGAGCGAACAGGAAAUCGGAAAGGCAACAGCAAAGUACU |
| | | UCUUCUACAGCAACAUCAUGAACUUCUUCAAGACAGAAAUCACACUGGCAAACGGAGAAAUCAGAAAGAGACCGCUGAUCGAAACAAACGG |
| | | AGAAACAGGAGAAAUCGUCUGGGACAAGGGAAGAGACUUCGCAACAGUCAGAAAGGUCCUGAGCAUGCCGCAGGUCAACAUCGUCAAGAAG |
| | | ACAGAAGUCCAGACAGGAGGAUUCAGCAAGGAAAGCAUCCUGCCGAAGAGAAACAGCGACAAGCUGAUCGCAAGAAAGAAGGACUGGGACC |
| | | CGAAGAAGUACGGAGGAUUCGACAGCCCGACAGUCGCAUACAGCGUCCUGGUCGUCGCAAAGGUCGAAAAGGGAAAGAGCAAGAAGCUGAA |
| | | GAGCGUCAAGGAACUGCUGGGAAUCACAAUCAUGGAAAGAAGCAGCUUCGAAAAGAACCCGAUCGACUUCCUGGAAGCAAAGGGAUACAAG |
| | | GAAGUCAAGAAGGACCUGAUCAUCAAGCUGCCGAAGUACAGCCUGUUCGAACUGGAAAACGGAAGAAAGAGAAUGCUGGCAAGCGCAGGAG |
| | | AACUGCAGAAGGGAAACGAACUGGCACUGCCGAGCAAGUACGUCAACUUCCUGUACCUGGCAAGCCACUACGAAAAGCUGAAGGGAAGCCC |
| | | GGAAGACAACGAACAGAAGCAGCUGUUCGUCGAACAGCACAAGCACUACCUGGACGAAAUCAUCGAACAGAUCAGCGAAUUCAGCAAGAGA |
| | | GUCAUCCUGGCAGACGCAAACCUGGACAAGGUCCUGAGCGCAUACAACAAGCACAGAGACAAGCCGAUCAGAGAACAGGCAGAAAACAUCA |
| | | UCCACCUGUUCACACUGACAAACCUGGGAGCACCGGCAGCAUUCAAGUACUUCGACACAACAAUCGACAGAAAGAGAUACACAAGCACAAA |
| | | GGAAGUCCUGGACGCAACACUGAUCCACCAGAGCAUCACAGGACUGUACGAAACAAGAAUCGACCUGAGCCAGCUGGGAGGAGACGGAGGA |
| | | GGAAGCCCGAAGAAGAAGAGAAAGGUCUAGCUAGCCAUCACAUUUAAAAGCAUCUCAGCCUACCAUGAGAAUAAGAGAAAGAAAAUGAAGA |
| | | UCAAUAGCUUAUUCAUCUCUUUUUCUUUUUCGUUGGUGUAAAGCCAACACCCUGUCUAAAAAACAUAAAUUUCUUUAAUCAUUUUGCCUCU |
| | | UUUCUCUGUGCUUCAAUUAAUAAAAAAUGGAAAGAACCUCGAGAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAUCUAG |
| 94 | SpyCas9 | AUGGACAAGAAGUACAGCAUCGGACUGGACAUCGGAACAAACAGCGUCGGAUGGGCAGUCAUCACAGACGAAUACAAGGUCCCGAGCAAGA |
| | (mRNA | AGUUCAAGGUCCUGGGAAACACAGACAGACACAGCAUCAAGAAGAACCUGAUCGGAGCACUGCUGUUCGACAGCGGAGAAACAGCAGAAGC |
| | X)- | AACAAGACUGAAGAGAACAGCAAGAAGAAGAUACACAAGAAGAAAGAACAGAAUCUGCUACCUGCAGGAAAUCUUCAGCAACGAAAUGGCA |
| | ORF | AAGGUCGACGACAGCUUCUUCCACAGACUGGAAGAAAGCUUCCUGGUCGAAGAAGACAAGAAGCACGAAAGACACCCGAUCUUCGGAAACA |
| | sequence | UCGUCGACGAAGUCGCAUACCACGAAAAGUACCCGACAAUCUACCACCUGAGAAAGAAGCUGGUCGACAGCACAGACAAGGCAGACCUGAG |
| | | ACUGAUCUACCUGGCACUGGCACACAUGAUCAAGUUCAGAGGACACUUCCUGAUCGAAGGAGACCUGAACCCGGACAACAGCGACGUCGAC |
| | | AAGCUGUUCAUCCAGCUGGUCCAGACAUACAACCAGCUGUUCGAAGAAAACCCGAUCAACGCAAGCGGAGUCGACGCAAAGGCAAUCCUGA |
| | | GCGCAAGACUGAGCAAGAGCAGAAGACUGGAAAACCUGAUCGCACAGCUGCCGGGAGAAAAGAAGAACGGACUGUUCGGAAACCUGAUCGC |
| | | ACUGAGCCUGGGACUGACACCGAACUUCAAGAGCAACUUCGACCUGGCAGAAGACGCAAAGCUGCAGCUGAGCAAGGACACAUACGACGAC |
| | | GACCUGGACAACCUGCUGGCACAGAUCGGAGACCAGUACGCAGACCUGUUCCUGGCAGCAAAGAACCUGAGCGACGCAAUCCUGCUGAGCG |
| | | ACAUCCUGAGAGUCAACACAGAAAUCACAAAGGCACCGCUGAGCGCAAGCAUGAUCAAGAGAUACGACGAACACCACCAGGACCUGACACU |
| | | GCUGAAGGCACUGGUCAGACAGCAGCUGCCGGAAAAGUACAAGGAAAUCUUCUUCGACCAGAGCAAGAACGGAUACGCAGGAUACAUCGAC |
| | | GGAGGAGCAAGCCAGGAAGAAUUCUACAAGUUCAUCAAGCCGAUCCUGGAAAAGAUGGACGGAACAGAAGAACUGCUGGUCAAGCUGAACA |
| | | GAGAAGACCUGCUGAGAAAGCAGAGAACAUUCGACAACGGAAGCAUCCCGCACCAGAUCCACCUGGGAGAACUGCACGCAAUCCUGAGAAG |
| | | ACAGGAAGACUUCUACCCGUUCCUGAAGGACAACAGAGAAAAGAUCGAAAAGAUCCUGACAUUCAGAAUCCCGUACUACGUCGGACCGCUG |
| | | GCAAGAGGAAACAGCAGAUUCGCAUGGAUGACAAGAAAGAGCGAAGAAACAAUCACACCGUGGAACUUCGAAGAAGUCGUCGACAAGGGAG |
| | | CAAGCGCACAGAGCUUCAUCGAAAGAAUGACAAACUUCGACAAGAACCUGCCGAACGAAAAGGUCCUGCCGAAGCACAGCCUGCUGUACGA |
| | | AUACUUCACAGUCUACAACGAACUGACAAAGGUCAAGUACGUCACAGAAGGAAUGAGAAAGCCGGCAUUCCUGAGCGGAGAACAGAAGAAG |
| | | GCAAUCGUCGACCUGCUGUUCAAGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGAUCAUCAAGGACAAGGACUUCCU |
| | | GGACAACGAAGAAAACGAAGACAUCCUGGAAGACAUCGUCCUGACACUGACACUGUUCGAAGACAGAGAAAUGAUCGAAGAAAGACUGAAG |
| | | ACAUACGCACACCUGUUCGACGACAAGGUCAUGAAGCAGCUGAAGAGAAGAAGAUACACAGGAUGGGGAAGACUGAGCAGAAAGCUGAUCA |
| | | ACGGAAUCAGAGACAAGCAGAGCGGAAAGACAAUCCUGGACUUCCUGAAGAGCGACGGAUUCGCAAACAGAAACUUCAUGCAGCUGAUCCA |
| | | CGACGACAGCCUGACAUUCAAGGAAGACAUCCAGAAGGCACAGGUCAGCGGACAGGGAGACAGCCUGCACGAACACAUCGCAAACCUGGCA |
| | | GGAAGCCCGGCAAUCAAGAAGGGAAUCCUGCAGACAGUCAAGGUCGUCGACGAACUGGUCAAGGUCAUGGGAAGACACAAGCCGGAAAACA |
| | | UCGUCAUCGAAAUGGCAAGAGAAAACCAGACAACACAGAAGGGACAGAAGAACAGCAGAGAAAGAAUGAAGAGAAUCGAAGAAGGAAUCAA |
| | | GGAACUGGGAAGCCAGAUCCUGAAGGAACACCCGGUCGAAAACACACAGCUGCAGAACGAAAAGCUGUACCUGUACUACCUGCAGAACGGA |
| | | AGAGACAUGUACGUCGACCAGGAACUGGACAUCAACAGACUGAGCGACUACGACGUCGACCACAUCGUCCCGCAGAGCUUCCUGAAGGACG |
| | | ACAGCAUCGACAACAAGGUCCUGACAAGAAGCGACAAGAACAGAGGAAAGAGCGACAACGUCCCGAGCGAAGAAGUCGUCAAGAAGAUGAA |
| | | GAACUACUGGAGACAGCUGCUGAACGCAAAGCUGAUCACACAGAGAAAGUUCGACAACCUGACAAAGGCAGAGAGAGGAGGACUGAGCGAA |
| | | CUGGACAAGGCAGGAUUCAUCAAGAGACAGCUGGUCGAAACAAGACAGAUCACAAAGCACGUCGCACAGAUCCUGGACAGCAGAAUGAACA |
| | | CAAAGUACGACGAAAACGACAAGCUGAUCAGAGAAGUCAAGGUCAUCACACUGAAGAGCAAGCUGGUCAGCGACUUCAGAAAGGACUUCCA |
| | | GUUCUACAAGGUCAGAGAAAUCAACAACUACCACCACGCACACGACGCAUACCUGAACGCAGUCGUCGGAACAGCACUGAUCAAGAAGUAC |
| | | CCGAAGCUGGAAAGCGAAUUCGUCUACGGAGACUACAAGGUCUACGACGUCAGAAAGAUGAUCGCAAAGAGCGAACAGGAAAUCGGAAAGG |
| | | CAACAGCAAAGUACUUCUUCUACAGCAACAUCAUGAACUUCUUCAAGACAGAAAUCACACUGGCAAACGGAGAAAUCAGAAAGAGACCGCU |
| | | GAUCGAAACAAACGGAGAAACAGGAGAAAUCGUCUGGGACAAGGGAAGAGACUUCGCAACAGUCAGAAAGGUCCUGAGCAUGCCGCAGGUC |
| | | AACAUCGUCAAGAAGACAGAAGUCCAGACAGGAGGAUUCAGCAAGGAAAGCAUCCUGCCGAAGAGAAACAGCGACAAGCUGAUCGCAAGAA |
| | | AGAAGGACUGGGACCCGAAGAAGUACGGAGGAUUCGACAGCCCGACAGUCGCAUACAGCGUCCUGGUCGUCGCAAAGGUCGAAAAGGGAAA |
| | | GAGCAAGAAGCUGAAGAGCGUCAAGGAACUGCUGGGAAUCACAAUCAUGGAAAGAAGCAGCUUCGAAAAGAACCCGAUCGACUUCCUGGAA |
| | | GCAAAGGGAUACAAGGAAGUCAAGAAGGACCUGAUCAUCAAGCUGCCGAAGUACAGCCUGUUCGAACUGGAAAACGGAAGAAAGAGAAUGC |
| | | UGGCAAGCGCAGGAGAACUGCAGAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGACGAAAUCAUCGAACAGAUCAGC |
| | | GAAUUCAGCAAGAGAGUCAUCCUGGCAGACGCAAACCUGGACAAGGUCCUGAGCGCAUACAACAAGCACAGAGACAAGCCGAUCAGAGAAC |
| | | AGGCAGAAAACAUCAUCCACCUGUUCACACUGACAAACCUGGGAGCACCGGCAGCAUUCAAGUACUUCGACACAACAAUCGACAGAAAGAG |
| | | AUACACAAGCACAAAGGAAGUCCUGGACGCAACACUGAUCCACCAGAGCAUCACAGGACUGUACGAAACAAGAAUCGACCUGAGCCAGCUG |
| | | GGAGGAGACGGAGGAGGAAGCCCGAAGAAGAAGAGAAAGGUCUAG |
| 95 | SpyCas9 | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | (mRNA | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | X)- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | amino | DLDNLLAQIGDQYADLFLAAKNLSDAILLSDILRVNTEITKAPLSASMIKRYDEHHQDLTLLKALVRQQLPEKYKEIFFDQSKNGYAGYID |
| | acid | GGASQEEFYKFIKPILEKMDGTEELLVKLNREDLLRKQRTFDNGSIPHQIHLGELHAILRRQEDFYPFLKDNREKIEKILTFRIPYYVGPL |
| | sequence | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGTYHDLLKIIKDKDFLDNEENEDILEDIVLTLTLFEDREMIEERLK |
| | | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKRYTSTKEVLDATLIHQSITGLYETRIDLSQL |
| | | GGDGGGSPKKKRKV* |
| 96 | SpyCas9- | AUGGACAAGAAGUACAGCAUCGGACUGGACAUCGGAACAAACAGCGUCGGAUGGGCAGUCAUCACAGACGAAUACAA |
| | Nme | GGUCCCGAGCAAGAAGUUCAAGGUCCUGGGAAACACAGACAGACACAGCAUCAAGAAGAACCUGAUCGGAGCACUGC |
| | cleavase | UGUUCGACAGCGGAGAAACAGCAGAAGCAACAAGACUGAAGAGAACAGCAAGAAGAAGAUACACAAGAAGAAAGAAC |
| | fusion | AGAAUCUGCUACCUGCAGGAAAUCUUCAGCAACGAAAUGGCAAAGGUCGACGACAGCUUCUUCCACAGACUGGAAGA |
| | ORF | AAGCUUCCUGGUCGAAGAAGACAAGAAGCACGAAAGACACCCGAUCUUCGGAAACAUCGUCGACGAAGUCGCAUACC |
| | | ACGAAAAGUACCCGACAAUCUACCACCUGAGAAAGAAGCUGGUCGACAGCACAGACAAGGCAGACCUGAGACUGAUC |
| | | UACCUGGCACUGGCACACAUGAUCAAGUUCAGAGGACACUUCCUGAUCGAAGGAGACCUGAACCCGGACAACAGCGAC |
| | | GUCGACAAGCUGUUCAUCCAGCUGGUCCAGACAUACAACCAGCUGUUCGAAGAAAACCCGAUCAACGCAAGCGGAGUC |
| | | GACGCAAAGGCAAUCCUGAGCGCAAGACUGAGCAAGAGCAGAAGACUGGAAAACCUGAUCGCACAGCUGCCGGGAGA |
| | | AAAGAAGAACGGACUGUUCGGAAACCUGAUCGCACUGAGCCUGGGACUGACACCGAACUUCAAGAGCAACUUCGACC |
| | | UGGCAGAAGACGCAAAGCUGCAGCUGAGCAAGGACACAUACGACGACGACCUGGACAACCUGCUGGCACAGAUCGGA |
| | | GACCAGUACGCAGACCUGUUCCUGGCAGCAAAGAACCUGAGCGACGCAAUCCUGCUGAGCGACAUCCUGAGAGUCAAC |
| | | ACAGAAAUCACAAAGGCACCGCUGAGCGCAAGCAUGAUCAAGAGAUACGACGAACACCACCAGGACCUGACACUGCUG |
| | | AAGGCACUGGUCAGACAGCAGCUGCCGGAAAAGUACAAGGAAAUCUUCUUCGACCAGAGCAAGAACGGAUACGCAGG |
| | | AUACAUCGACGGAGGAGCAAGCCAGGAAGAAUUCUACAAGUUCAUCAAGCCGAUCCUGGAAAAGAUGGACGGAACAG |
| | | AAGAACUGCUGGUCAAGCUGAACAGAGAAGACCUGCUGAGAAAGCAGAGAACAUUCGACAACGGAAGCAUCCCGCAC |
| | | CAGAUCCACCUGGGAGAACUGCACGCAAUCCUGAGAAGACAGGAAGACUUCUACCCGUUCCUGAAGGACAACAGAGA |
| | | AAAGAUCGAAAAGAUCCUGACAUUCAGAAUCCCGUACUACGUCGGACCGCUGGCAAGAGGAAACAGCAGAUUCGCAU |
| | | GGAUGACAAGAAAGAGCGAAGAAACAAUCACACCGUGGAACUUCGAAGAAGUCGUCGACAAGGGAGCAAGCGCACAG |
| | | AGCUUCAUCGAAAGAAUGACAAACUUCGACAAGAACCUGCCGAACGAAAAGGUCCUGCCGAAGCACAGCCUGCUGUAC |
| | | GAAUACUUCACAGUCUACAACGAACUGACAAAGGUCAAGUACGUCACAGAAGGAAUGAGAAAGCCGGCAUUCCUGAG |
| | | CGGAGAACAGAAGAAGGCAAUCGUCGACCUGCUGUUCAAGACAAACAGAAAGGUCACAGUCAAGCAGCUGAAGGAAG |
| | | ACUACUUCAAGAAGAUCGAAUGCUUCGACAGCGUCGAAAUCAGCGGAGUCGAAGACAGAUUCAACGCAAGCCUGGGA |
| | | ACAUACCACGACCUGCUGAAGAUCAUCAAGGACAAGGACUUCCUGGACAACGAAGAAAACGAAGACAUCCUGGAAGA |
| | | CAUCGUCCUGACACUGACACUGUUCGAAGACAGAGAAAUGAUCGAAGAAAGACUGAAGACAUACGCACACCUGUUCG |
| | | ACGACAAGGUCAUGAAGCAGCUGAAGAGAAGAAGAUACACAGGAUGGGGAAGACUGAGCAGAAAGCUGAUCAACGGA |
| | | AUCAGAGACAAGCAGAGCGGAAAGACAAUCCUGGACUUCCUGAAGAGCGACGGAUUCGCAAACAGAAACUUCAUGCA |
| | | GCUGAUCCACGACGACAGCCUGACAUUCAAGGAAGACAUCCAGAAGGCACAGGUCAGCGGACAGGGAGACAGCCUGCA |
| | | CGAACACAUCGCAAACCUGGCAGGAAGCCCGGCAAUCAAGAAGGGAAUCCUGCAGACAGUCAAGGUCGUCGACGAACU |
| | | GGUCAAGGUCAUGGGAAGACACAAGCCGGAAAACAUCGUCAUCGAAAUGGCAAGAGAAAACCAGACAACACAGAAGG |
| | | GACAGAAGAACAGCAGAGAAAGAAUGAAGAGAAUCGAAGAAGGAAUCAAGGAACUGGGAAGCCAGAUCCUGAAGGAA |
| | | CACCCGGUCGAAAACACACAGCUGCAGAACGAAAAGCUGUACCUGUACUACCUGCAGAACGGAAGAGACAUGUACGUC |
| | | GACCAGGAACUGGACAUCAACAGACUGAGCGACUACGACGUCGACCACAUCGUCCCGCAGAGCUUCCUGAAGGACGAC |
| | | AGCAUCGACAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAACCUGACAA |
| | | AGGCAGAGAGAGGAGGACUGAGCGAACUGGACAAGGCAGGAUUCAUCAAGAGACAGCUGGUCGAAACAAGACAGAUC |
| | | ACAAAGCACGUCGCACAGAUCCUGGACAGCAGAAUGAACACAAAGUACGACGAAAACGACAAGCUGAUCAGAGAAGU |
| | | CAAGGUCAUCACACUGAAGAGCAAGCUGGUCAGCGACUUCAGAAAGGACUUCCAGUUCUACAAGGUCAGAGAAAUCA |
| | | ACAACUACCACCACGCACACGACGCAUACCUGAACGCAGUCGUCGGAACAGCACUGAUCAAGAAGUACCCGAAGCUGG |
| | | AAAGCGAAUUCGUCUACGGAGACUACAAGGUCUACGACGUCAGAAAGAUGAUCGCAAAGAGCGAACAGGAAAUCGGA |
| | | AAGGCAACAGCAAAGUACUUCUUCUACAGCAACAUCAUGAACUUCUUCAAGACAGAAAUCACACUGGCAAACGGAGA |
| | | AAUCAGAAAGAGACCGCUGAUCGAAACAAACGGAGAAACAGGAGAAAUCGUCUGGGACAAGGGAAGAGACUUCGCAA |
| | | CAGUCAGAAAGGUCCUGAGCAUGCCGCAGGUCAACAUCGUCAAGAAGACAGAAGUCCAGACAGGAGGAUUCAGCAAG |
| | | GAAAGCAUCCUGCCGAAGAGAAACAGCGACAAGCUGAUCGCAAGAAAGAAGGACUGGGACCCGAAGAAGUACGGAGG |
| | | AUUCGACAGCCCGACAGUCGCAUACAGCGUCCUGGUCGUCGCAAAGGUCGAAAAGGGAAAGAGCAAGAAGCUGAAGA |
| | | GCGUCAAGGAACUGCUGGGAAUCACAAUCAUGGAAAGAAGCAGCUUCGAAAAGAACCCGAUCGACUUCCUGGAAGCA |
| | | AAGGGAUACAAGGAAGUCAAGAAGGACCUGAUCAUCAAGCUGCCGAAGUACAGCCUGUUCGAACUGGAAAACGGAAG |
| | | AAAGAGAAUGCUGGCAAGCGCAGGAGAACUGCAGAAGGGAAACGAACUGGCACUGCCGAGCAAGUACGUCAACUUCC |
| | | UGUACCUGGCAAGCCACUACGAAAAGCUGAAGGGAAGCCCGGAAGACAACGAACAGAAGCAGCUGUUCGUCGAACAG |
| | | CACAAGCACUACCUGGACGAAAUCAUCGAACAGAUCAGCGAAUUCAGCAAGAGAGUCAUCCUGGCAGACGCAAACCUG |
| | | GACAAGGUCCUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUACACAAGCACA |
| | | AAGGAAGUCCUGGACGCAACACUGAUCCACCAGAGCAUCACAGGACUGUACGAAACAAGAAUCGACCUGAGCCAGCUG |
| | | GGAGGAGACGGCGCGCCUGAGUCUGCUACAGAAAGUGGGGGAACCUCCACAGAGUCUGAAGGAAGUGCCGGCACUUC |
| | | AACCGAGAGCGAAGGGUCCGCUGGAUCUGCAGGCUCCACAUCUACUGAGGAAGGCACCAGUACAGAGUCAGAAGGCA |
| | | GCGCCGGAACUUCCACCGAGUCUGAAGGCAGUGCCGGGACAAGUGAAUCAGCUACUGAAAGCGGCGGGACAUCAACU |
| | | GAGAGCGAAGGAUCCAGUUCCGGAGCAGCAUUCAAGCCGAACUCGAUCAACUACAUCCUGGGACUGGACAUCGGAAU |
| | | CGCAUCGGUCGGAUGGGCAAUGGUCGAAAUCGACGAAGAAGAAAACCCGAUCAGACUGAUCGACCUGGGAGUCAGAG |
| | | UCUUCGAAAGAGCAGAAGUCCCGAAGACAGGAGACUCGCUGGCAAUGGCAAGAAGACUGGCAAGAUCGGUCAGAAGA |
| | | CUGACAAGAAGAAGAGCACACAGACUGCUGAGAACAAGAAGACUGCUGAAGAGAGAAGGAGUCCUGCAGGCAGCAAA |
| | | CUUCGACGAAAACGGACUGAUCAAGUCGCUGCCGAACACACCGUGGCAGCUGAGAGCAGCAGCACUGGACAGAAAGCU |
| | | GACACCGCUGGAAUGGUCGGCAGUCCUGCUGCACCUGAUCAAGCACAGAGGAUACCUGUCGCAGAGAAAGAACGAAG |
| | | GAGAAACAGCAGACAAGGAACUGGGAGCACUGCUGAAGGGAGUCGCAGGAAACGCACACGCACUGCAGACAGGAGAC |
| | | UUCAGAACACCGGCAGAACUGGCACUGAACAAGUUCGAAAAGGAAUCGGGACACAUCAGAAACCAGAGAUCGGACUA |
| | | CUCGCACACAUUCUCGAGAAAGGACCUGCAGGCAGAACUGAUCCUGCUGUUCGAAAAGCAGAAGGAAUUCGGAAACC |
| | | CGCACGUCUCGGGAGGACUGAAGGAAGGAAUCGAAACACUGCUGAUGACACAGAGACCGGCACUGUCGGGAGACGCA |
| | | GUCCAGAAGAUGCUGGGACACUGCACAUUCGAACCGGCAGAACCGAAGGCAGCAAAGAACACAUACACAGCAGAAAG |
| | | AUUCAUCUGGCUGACAAAGCUGAACAACCUGAGAAUCCUGGAACAGGGAUCGGAAAGACCGCUGACAGACACAGAAA |
| | | GAGCAACACUGAUGGACGAACCGUACAGAAAGUCGAAGCUGACAUACGCACAGGCAAGAAAGCUGCUGGGACUGGAA |
| | | GACACAGCAUUCUUCAAGGGACUGAGAUACGGAAAGGACAACGCAGAAGCAUCGACACUGAUGGAAAUGAAGGCAUA |
| | | CCACGCAAUCUCGAGAGCACUGGAAAAGGAAGGACUGAAGGACAAGAAGUCGCCGCUGAACCUGUCGCCGGAACUGC |
| | | AGGACGAAAUCGGAACAGCAUUCUCGCUGUUCAAGACAGACGAAGACAUCACAGGAAGACUGAAGGACAGAAUCCAG |
| | | CCGGAAAUCCUGGAAGCACUGCUGAAGCACAUCUCGUUCGACAAGUUCGUCCAGAUCUCGCUGAAGGCACUGAGAAG |
| | | AAUCGUCCCGCUGAUGGAACAGGGAAAGAGAUACGACGAAGCAUGCGCAGAAAUCUACGGAGACCACUACGGAAAGA |
| | | AGAACACAGAAGAAAAGAUCUACCUGCCGCCGAUCCCGGCAGACGAAAUCAGAAACCCGGUCGUCCUGAGAGCACUGU |
| | | CGCAGGCAAGAAAGGUCAUCAACGGAGUCGUCAGAAGAUACGGAUCGCCGGCAAGAAUCCACAUCGAAACAGCAAGA |
| | | GAAGUCGGAAAGUCGUUCAAGGACAGAAAGGAAAUCGAAAAGAGACAGGAAGAAAACAGAAAGGACAGAGAAAAGGC |
| | | AGCAGCAAAGUUCAGAGAAUACUUCCCGAACUUCGUCGGAGAACCGAAGUCGAAGGACAUCCUGAAGCUGAGACUGU |
| | | ACGAACAGCAGCACGGAAAGUGCCUGUACUCGGGAAAGGAAAUCAACCUGGGAAGACUGAACGAAAAGGGAUACGUC |
| | | GAAAUCGACCACGCACUGCCGUUCUCGAGAACAUGGGACGACUCGUUCAACAACAAGGUCCUGGUCCUGGGAUCGGAA |
| | | AACCAGAACAAGGGAAACCAGACACCGUACGAAUACUUCAACGGAAAGGACAACUCGAGAGAAUGGCAGGAAUUCAA |
| | | GGCAAGAGUCGAAACAUCGAGAUUCCCGAGAUCGAAGAAGCAGAGAAUCCUGCUGCAGAAGUUCGACGAAGACGGAU |
| | | UCAAGGAAAGAAACCUGAACGACACAAGAUACGUCAACAGAUUCCUGUGCCAGUUCGUCGCAGACAGAAUGAGACUG |
| | | ACAGGAAAGGGAAAGAAGAGAGUCUUCGCAUCGAACGGACAGAUCACAAACCUGCUGAGAGGAUUCUGGGGACUGAG |
| | | AAAGGUCAGAGCAGAAAACGACAGACACCACGCACUGGACGCAGUCGUCGUCGCAUGCUCGACAGUCGCAAUGCAGCA |
| | | GAAGAUCACAAGAUUCGUCAGAUACAAGGAAAUGAACGCAUUCGACGGAAAGACAAUCGACAAGGAAACAGGAGAAG |
| | | UCCUGCACCAGAAGACACACUUCCCGCAGCCGUGGGAAUUCUUCGCACAGGAAGUCAUGAUCAGAGUCUUCGGAAAGC |
| | | CGGACGGAAAGCCGGAAUUCGAAGAAGCAGACACACUGGAAAAGCUGAGAACACUGCUGGCAGAAAAGCUGUCGUCG |
| | | AGACCGGAAGCAGUCCACGAAUACGUCACACCGCUGUUCGUCUCGAGAGCACCGAACAGAAAGAUGUCGGGACAGGG |
| | | ACACAUGGAAACAGUCAAGUCGGCAAAGAGACUGGACGAAGGAGUCUCGGUCCUGAGAGUCCCGCUGACACAGCUGA |
| | | AGCUGAAGGACCUGGAAAAGAUGGUCAACAGAGAAAGAGAACCGAAGCUGUACGAAGCACUGAAGGCAAGACUGGAA |
| | | GCACACAAGGACGACCCGGCAAAGGCAUUCGCAGAACCGUUCUACAAGUACGACAAGGCAGGAAACAGAACACAGCAG |
| | | GUCAAGGCAGUCAGAGUCGAACAGGUCCAGAAGACAGGAGUCUGGGUCAGAAACCACAACGGAAUCGCAGACAACGC |
| | | AACAAUGGUCAGAGUAGACGUCUUCGAAAAGGGAGACAAGUACUACCUGGUCCCGAUCUACUCGUGGCAGGUCGCAA |
| | | AGGGAAUCCUGCCGGACAGAGCAGUCGUCCAGGGAAAGGACGAAGAAGACUGGCAGCUGAUCGACGACUCGUUCAAC |
| | | UUCAAGUUCUCGCUGCACCCGAACGACCUGGUCGAAGUCAUCACAAAGAAGGCAAGAAUGUUCGGAUACUUCGCAUC |
| | | GUGCCACAGAGGAACAGGAAACAUCAACAUCAGAAUCCACGACCUGGACCACAAGAUCGGAAAGAACGGAAUCCUGG |
| | | AAGGAAUCGGAGUCAAGACAGCACUGUCGUUCCAGAAGUACCAGAUCGACGAACUGGGAAAGGAAAUCAGACCGUGC |
| | | AGACUGAAGAAGAGACCGCCGGUCAGAGGGGGCAGCGGCGGGUCUAAGAGGACCGCCGAUGGGAGUGAGUUUGAAAG |
| | | CCCUAAAAAGAAAAGAAAAGUGGAAUGA |
| 97 | SpyCas9- | MDKKYSIGLDIGTNSVGWAVITDEYKVPSKKFKVLGNTDRHSIKKNLIGALLFDSGETAEATRLKRTARRRYTRRKNRICYLQEI |
| | Nme | FSNEMAKVDDSFFHRLEESFLVEEDKKHERHPIFGNIVDEVAYHEKYPTIYHLRKKLVDSTDKADLRLIYLALAHMIKFRGHFLI |
| | cleavase | EGDLNPDNSDVDKLFIQLVQTYNQLFEENPINASGVDAKAILSARLSKSRRLENLIAQLPGEKKNGLFGNLIALSLGLTPNFKSNF |
| | fusion | DLAEDAKLQLSKDTYDDDLDNLLAQIGDQYADLFLAAKNLSDAILLSDILRVNTEITKAPLSASMIKRYDEHHQDLTLLKALVR |
| | amino | QQLPEKYKEIFFDQSKNGYAGYIDGGASQEEFYKFIKPILEKMDGTEELLVKLNREDLLRKQRTFDNGSIPHQIHLGELHAILRRQ |
| | acid | EDFYPFLKDNREKIEKILTFRIPYYVGPLARGNSRFAWMTRKSEETITPWNFEEVVDKGASAQSFIERMTNFDKNLPNEKVLPKH |
| | sequence | SLLYEYFTVYNELTKVKYVTEGMRKPAFLSGEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLFDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVKVVDELVKVMGRHKPENIVIEMA |
| | | RENQTTQKGQKNSRERMKRIEEGIKELGSQILKEHPVENTQLQNEKLYLYYLQNGRDMYVDQELDINRLSDYDVDHIVPQSFL |
| | | KDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDY |
| | | KVYDVRKMIAKSEQEIGKATAKYFFYSNIMNFFKTEITLANGEIRKRPLIETNGETGEIVWDKGRDFATVRKVLSMPQVNIVKKT |
| | | EVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYL |
| | | DEIIEQISEFSKRVILADANLDKVLSAYNKHRDKPIREQAENIIHLFTLTNLGAPAAFKYFDTTIDRKRYTSTKEVLDATLIHQSITG |
| | | LYETRIDLSQLGGDGAPESATESGGTSTESEGSAGTSTESEGSAGSAGSTSTEEGTSTESEGSAGTSTESEGSAGTSESATESGGTS |
| | | TESEGSSSGAAFKPNSINYILGLDIGIASVGWAMVEIDEEENPIRLIDLGVRVFERAEVPKTGDSLAMARRLARSVRRLTRRRAHR |
| | | LLRTRRLLKREGVLQAANFDENGLIKSLPNTPWQLRAAALDRKLTPLEWSAVLLHLIKHRGYLSQRKNEGETADKELGALLKG |
| | | VAGNAHALQTGDFRTPAELALNKFEKESGHIRNQRSDYSHTFSRKDLQAELILLFEKQKEFGNPHVSGGLKEGIETLLMTQRPA |
| | | LSGDAVQKMLGHCTFEPAEPKAAKNTYTAERFIWLTKLNNLRILEQGSERPLTDTERATLMDEPYRKSKLTYAQARKLLGLED |
| | | TAFFKGLRYGKDNAEASTLMEMKAYHAISRALEKEGLKDKKSPLNLSPELQDEIGTAFSLFKTDEDITGRLKDRIQPEILEALLK |
| | | HISFDKFVQISLKALRRIVPLMEQGKRYDEACAEIYGDHYGKKNTEEKIYLPPIPADEIRNPVVLRALSQARKVINGVVRRYGSP |
| | | ARIHIETAREVGKSFKDRKEIEKRQEENRKDREKAAAKFREYFPNFVGEPKSKDILKLRLYEQQHGKCLYSGKEINLGRLNEKG |
| | | YVEIDHALPFSRTWDDSFNNKVLVLGSENQNKGNQTPYEYFNGKDNSREWQEFKARVETSRFPRSKKQRILLQKFDEDGFKER |
| | | NLNDTRYVNRFLCQFVADRMRLTGKGKKRVFASNGQITNLLRGFWGLRKVRAENDRHHALDAVVVACSTVAMQQKITRFVR |
| | | YKEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYD |
| | | KAGNRTQQVKAVRVEQVQKTGVWVRNHNGIADNATMVRVDVFEKGDKYYLVPIYSWQVAKGILPDRAVVQGKDEEDWQLI |
| | | DDSFNFKFSLHPNDLVEVITKKARMFGYFASCHRGTGNINIRIHDLDHKIGKNGILEGIGVKTALSFQKYQIDELGKEIRPCRLKK |
| | | RPPVRGGSGGSKRTADGSEFESPKKKRKVE* |
| 98 | Spy- | AUGGACAAGAAGUACAGCAUCGGACUGGACAUCGGAACAAACAGCGUCGGAUGGGCAGUCAUCACAGACGAAUACAAGGUCCCGAGCAAGA |
| | R1335S | AGUUCAAGGUCCUGGGAAACACAGACAGACACAGCAUCAAGAAGAACCUGAUCGGAGCACUGCUGUUCGACAGCGGAGAAACAGCAGAAGC |
| | Cas9- | AACAAGACUGAAGAGAACAGCAAGAAGAAGAUACACAAGAAGAAAGAACAGAAUCUGCUACCUGCAGGAAAUCUUCAGCAACGAAAUGGCA |
| | WT | AAGGUCGACGACAGCUUCUUCCACAGACUGGAAGAAAGCUUCCUGGUCGAAGAAGACAAGAAGCACGAAAGACACCCGAUCUUCGGAAACA |
| | NmeCas9 | UCGUCGACGAAGUCGCAUACCACGAAAAGUACCCGACAAUCUACCACCUGAGAAAGAAGCUGGUCGACAGCACAGACAAGGCAGACCUGAG |
| | fusion | ACUGAUCUACCUGGCACUGGCACACAUGAUCAAGUUCAGAGGACACUUCCUGAUCGAAGGAGACCUGAACCCGGACAACAGCGACGUCGAC |
| | ORF | AAGCUGUUCAUCCAGCUGGUCCAGACAUACAACCAGCUGUUCGAAGAAAACCCGAUCAACGCAAGCGGAGUCGACGCAAAGGCAAUCCUGA |
| | | GCGCAAGACUGAGCAAGAGCAGAAGACUGGAAAACCUGAUCGCACAGCUGCCGGGAGAAAAGAAGAACGGACUGUUCGGAAACCUGAUCGC |
| | | ACUGAGCCUGGGACUGACACCGAACUUCAAGAGCAACUUCGACCUGGCAGAAGACGCAAAGCUGCAGCUGAGCAAGGACACAUACGACGAC |
| | | GACCUGGACAACCUGCUGGCACAGAUCGGAGACCAGUACGCAGACCUGUUCCUGGCAGCAAAGAACCUGAGCGACGCAAUCCUGCUGAGCG |
| | | ACAUCCUGAGAGUCAACACAGAAAUCACAAAGGCACCGCUGAGCGCAAGCAUGAUCAAGAGAUACGACGAACACCACCAGGACCUGACACU |
| | | GCUGAAGGCACUGGUCAGACAGCAGCUGCCGGAAAAGUACAAGGAAAUCUUCUUCGACCAGAGCAAGAACGGAUACGCAGGAUACAUCGAC |
| | | GGAGGAGCAAGCCAGGAAGAAUUCUACAAGUUCAUCAAGCCGAUCCUGGAAAAGAUGGACGGAACAGAAGAACUGCUGGUCAAGCUGAACA |
| | | GAGAAGACCUGCUGAGAAAGCAGAGAACAUUCGACAACGGAAGCAUCCCGCACCAGAUCCACCUGGGAGAACUGCACGCAAUCCUGAGAAG |
| | | ACAGGAAGACUUCUACCCGUUCCUGAAGGACAACAGAGAAAAGAUCGAAAAGAUCCUGACAUUCAGAAUCCCGUACUACGUCGGACCGCUG |
| | | GCAAGAGGAAACAGCAGAUUCGCAUGGAUGACAAGAAAGAGCGAAGAAACAAUCACACCGUGGAACUUCGAAGAAGUCGUCGACAAGGGAG |
| | | CAAGCGCACAGAGCUUCAUCGAAAGAAUGACAAACUUCGACAAGAACCUGCCGAACGAAAAGGUCCUGCCGAAGCACAGCCUGCUGUACGA |
| | | AUACUUCACAGUCUACAACGAACUGACAAAGGUCAAGUACGUCACAGAAGGAAUGAGAAAGCCGGCAUUCCUGAGCGGAGAACAGAAGAAG |
| | | GCAAUCGUCGACCUGCUGUUCAAGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGAUCAUCAAGGACAAGGACUUCCU |
| | | GGACAACGAAGAAAACGAAGACAUCCUGGAAGACAUCGUCCUGACACUGACACUGUUCGAAGACAGAGAAAUGAUCGAAGAAAGACUGAAG |
| | | ACAUACGCACACCUGUUCGACGACAAGGUCAUGAAGCAGCUGAAGAGAAGAAGAUACACAGGAUGGGGAAGACUGAGCAGAAAGCUGAUCA |
| | | ACGGAAUCAGAGACAAGCAGAGCGGAAAGACAAUCCUGGACUUCCUGAAGAGCGACGGAUUCGCAAACAGAAACUUCAUGCAGCUGAUCCA |
| | | CGACGACAGCCUGACAUUCAAGGAAGACAUCCAGAAGGCACAGGUCAGCGGACAGGGAGACAGCCUGCACGAACACAUCGCAAACCUGGCA |
| | | GGAAGCCCGGCAAUCAAGAAGGGAAUCCUGCAGACAGUCAAGGUCGUCGACGAACUGGUCAAGGUCAUGGGAAGACACAAGCCGGAAAACA |
| | | UCGUCAUCGAAAUGGCAAGAGAAAACCAGACAACACAGAAGGGACAGAAGAACAGCAGAGAAAGAAUGAAGAGAAUCGAAGAAGGAAUCAA |
| | | GGAACUGGGAAGCCAGAUCCUGAAGGAACACCCGGUCGAAAACACACAGCUGCAGAACGAAAAGCUGUACCUGUACUACCUGCAGAACGGA |
| | | AGAGACAUGUACGUCGACCAGGAACUGGACAUCAACAGACUGAGCGACUACGACGUCGACCACAUCGUCCCGCAGAGCUUCCUGAAGGACG |
| | | ACAGCAUCGACAACAAGGUCCUGACAAGAAGCGACAAGAACAGAGGAAAGAGCGACAACGUCCCGAGCGAAGAAGUCGUCAAGAAGAUGAA |
| | | GAACUACUGGAGACAGCUGCUGAACGCAAAGCUGAUCACACAGAGAAAGUUCGACAACCUGACAAAGGCAGAGAGAGGAGGACUGAGCGAA |
| | | CUGGACAAGGCAGGAUUCAUCAAGAGACAGCUGGUCGAAACAAGACAGAUCACAAAGCACGUCGCACAGAUCCUGGACAGCAGAAUGAACA |
| | | CAAAGUACGACGAAAACGACAAGCUGAUCAGAGAAGUCAAGGUCAUCACACUGAAGAGCAAGCUGGUCAGCGACUUCAGAAAGGACUUCCA |
| | | GUUCUACAAGGUCAGAGAAAUCAACAACUACCACCACGCACACGACGCAUACCUGAACGCAGUCGUCGGAACAGCACUGAUCAAGAAGUAC |
| | | CCGAAGCUGGAAAGCGAAUUCGUCUACGGAGACUACAAGGUCUACGACGUCAGAAAGAUGAUCGCAAAGAGCGAACAGGAAAUCGGAAAGG |
| | | CAACAGCAAAGUACUUCUUCUACAGCAACAUCAUGAACUUCUUCAAGACAGAAAUCACACUGGCAAACGGAGAAAUCAGAAAGAGACCGCU |
| | | GAUCGAAACAAACGGAGAAACAGGAGAAAUCGUCUGGGACAAGGGAAGAGACUUCGCAACAGUCAGAAAGGUCCUGAGCAUGCCGCAGGUC |
| | | AACAUCGUCAAGAAGACAGAAGUCCAGACAGGAGGAUUCAGCAAGGAAAGCAUCCUGCCGAAGAGAAACAGCGACAAGCUGAUCGCAAGAA |
| | | AGAAGGACUGGGACCCGAAGAAGUACGGAGGAUUCGACAGCCCGACAGUCGCAUACAGCGUCCUGGUCGUCGCAAAGGUCGAAAAGGGAAA |
| | | GAGCAAGAAGCUGAAGAGCGUCAAGGAACUGCUGGGAAUCACAAUCAUGGAAAGAAGCAGCUUCGAAAAGAACCCGAUCGACUUCCUGGAA |
| | | GCAAAGGGAUACAAGGAAGUCAAGAAGGACCUGAUCAUCAAGCUGCCGAAGUACAGCCUGUUCGAACUGGAAAACGGAAGAAAGAGAAUGC |
| | | UGGCAAGCGCAGGAGAACUGCAGAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGACGAAAUCAUCGAACAGAUCAGC |
| | | GAAUUCAGCAAGAGAGUCAUCCUGGCAGACGCAAACCUGGACAAGGUCCUGAGCGCAUACAACAAGCACAGAGACAAGCCGAUCAGAGAAC |
| | | AGGCAGAAAACAUCAUCCACCUGUUCACACUGACAAACCUGGGAGCACCGGCAGCAUUCAAGUACUUCGACACAACAAUCGACAGAAAGuC |
| | | AUACACAAGCACAAAGGAAGUCCUGGACGCAACACUGAUCCACCAGAGCAUCACAGGACUGUACGAAACAAGAAUCGACCUGAGCCAGCUG |
| | | GGAGGAGACGGCGCGCCUGAGUCUGCUACAGAAAGUGGGGGAACCUCCACAGAGUCUGAAGGAAGUGCCGGCACUUCAACCGAGAGCGAAG |
| | | GGUCCGCUGGAUCUGCAGGCUCCACAUCUACUGAGGAAGGCACCAGUACAGAGUCAGAAGGCAGCGCCGGAACUUCCACCGAGUCUGAAGG |
| | | CAGUGCCGGGACAAGUGAAUCAGCUACUGAAAGCGGCGGGACAUCAACUGAGAGCGAAGGAUCCAGUUCCGGAGCAGCAUUCAAGCCGAAC |
| | | UCGAUCAACUACAUCCUGGGACUGGACAUCGGAAUCGCAUCGGUCGGAUGGGCAAUGGUCGAAAUCGACGAAGAAGAAAACCCGAUCAGAC |
| | | UGAUCGACCUGGGAGUCAGAGUCUUCGAAAGAGCAGAAGUCCCGAAGACAGGAGACUCGCUGGCAAUGGCAAGAAGACUGGCAAGAUCGGU |
| | | CAGAAGACUGACAAGAAGAAGAGCACACAGACUGCUGAGAACAAGAAGACUGCUGAAGAGAGAAGGAGUCCUGCAGGCAGCAAACUUCGAC |
| | | GAAAACGGACUGAUCAAGUCGCUGCCGAACACACCGUGGCAGCUGAGAGCAGCAGCACUGGACAGAAAGCUGACACCGCUGGAAUGGUCGG |
| | | CAGUCCUGCUGCACCUGAUCAAGCACAGAGGAUACCUGUCGCAGAGAAAGAACGAAGGAGAAACAGCAGACAAGGAACUGGGAGCACUGCU |
| | | GAAGGGAGUCGCAGGAAACGCACACGCACUGCAGACAGGAGACUUCAGAACACCGGCAGAACUGGCACUGAACAAGUUCGAAAAGGAAUCG |
| | | GGACACAUCAGAAACCAGAGAUCGGACUACUCGCACACAUUCUCGAGAAAGGACCUGCAGGCAGAACUGAUCCUGCUGUUCGAAAAGCAGA |
| | | AGGAAUUCGGAAACCCGCACGUCUCGGGAGGACUGAAGGAAGGAAUCGAAACACUGCUGAUGACACAGAGACCGGCACUGUCGGGAGACGC |
| | | AGUCCAGAAGAUGCUGGGACACUGCACAUUCGAACCGGCAGAACCGAAGGCAGCAAAGAACACAUACACAGCAGAAAGAUUCAUCUGGCUG |
| | | ACAAAGCUGAACAACCUGAGAAUCCUGGAACAGGGAUCGGAAAGACCGCUGACAGACACAGAAAGAGCAACACUGAUGGACGAACCGUACA |
| | | GAAAGUCGAAGCUGACAUACGCACAGGCAAGAAAGCUGCUGGGACUGGAAGACACAGCAUUCUUCAAGGGACUGAGAUACGGAAAGGACAA |
| | | CGCAGAAGCAUCGACACUGAUGGAAAUGAAGGCAUACCACGCAAUCUCGAGAGCACUGGAAAAGGAAGGACUGAAGGACAAGAAGUCGCCG |
| | | CUGAACCUGUCGCCGGAACUGCAGGACGAAAUCGGAACAGCAUUCUCGCUGUUCAAGACAGACGAAGACAUCACAGGAAGACUGAAGGACA |
| | | GAAUCCAGCCGGAAAUCCUGGAAGCACUGCUGAAGCACAUCUCGUUCGACAAGUUCGUCCAGAUCUCGCUGAAGGCACUGAGAAGAAUCGU |
| | | CCCGCUGAUGGAACAGGGAAAGAGAUACGACGAAGCAUGCGCAGAAAUCUACGGAGACCACUACGGAAAGAAGAACACAGAAGAAAAGAUC |
| | | UACCUGCCGCCGAUCCCGGCAGACGAAAUCAGAAACCCGGUCGUCCUGAGAGCACUGUCGCAGGCAAGAAAGGUCAUCAACGGAGUCGUCA |
| | | GAAGAUACGGAUCGCCGGCAAGAAUCCACAUCGAAACAGCAAGAGAAGUCGGAAAGUCGUUCAAGGACAGAAAGGAAAUCGAAAAGAGACA |
| | | GGAAGAAAACAGAAAGGACAGAGAAAAGGCAGCAGCAAAGUUCAGAGAAUACUUCCCGAACUUCGUCGGAGAACCGAAGUCGAAGGACAUC |
| | | CUGAAGCUGAGACUGUACGAACAGCAGCACGGAAAGUGCCUGUACUCGGGAAAGGAAAUCAACCUGGGAAGACUGAACGAAAAGGGAUACG |
| | | UCGAAAUCGACCACGCACUGCCGUUCUCGAGAACAUGGGACGACUCGUUCAACAACAAGGUCCUGGUCCUGGGAUCGGAAAACCAGAACAA |
| | | GGGAAACCAGACACCGUACGAAUACUUCAACGGAAAGGACAACUCGAGAGAAUGGCAGGAAUUCAAGGCAAGAGUCGAAACAUCGAGAUUC |
| | | CCGAGAUCGAAGAAGCAGAGAAUCCUGCUGCAGAAGUUCGACGAAGACGGAUUCAAGGAAAGAAACCUGAACGACACAAGAUACGUCAACA |
| | | GAUUCCUGUGCCAGUUCGUCGCAGACAGAAUGAGACUGACAGGAAAGGGAAAGAAGAGAGUCUUCGCAUCGAACGGACAGAUCACAAACCU |
| | | GCUGAGAGGAUUCUGGGGACUGAGAAAGGUCAGAGCAGAAAACGACAGACACCACGCACUGGACGCAGUCGUCGUCGCAUGCUCGACAGUC |
| | | GCAAUGCAGCAGAAGAUCACAAGAUUCGUCAGAUACAAGGAAAUGAACGCAUUCGACGGAAAGACAAUCGACAAGGAAACAGGAGAAGUCC |
| | | UGCACCAGAAGACACACUUCCCGCAGCCGUGGGAAUUCUUCGCACAGGAAGUCAUGAUCAGAGUCUUCGGAAAGCCGGACGGAAAGCCGGA |
| | | AUUCGAAGAAGCAGACACACUGGAAAAGCUGAGAACACUGCUGGCAGAAAAGCUGUCGUCGAGACCGGAAGCAGUCCACGAAUACGUCACA |
| | | CCGCUGUUCGUCUCGAGAGCACCGAACAGAAAGAUGUCGGGACAGGGACACAUGGAAACAGUCAAGUCGGCAAAGAGACUGGACGAAGGAG |
| | | UCUCGGUCCUGAGAGUCCCGCUGACACAGCUGAAGCUGAAGGACCUGGAAAAGAUGGUCAACAGAGAAAGAGAACCGAAGCUGUACGAAGC |
| | | ACUGAAGGCAAGACUGGAAGCACACAAGGACGACCCGGCAAAGGCAUUCGCAGAACCGUUCUACAAGUACGACAAGGCAGGAAACAGAACA |
| | | CAGCAGGUCAAGGCAGUCAGAGUCGAACAGGUCCAGAAGACAGGAGUCUGGGUCAGAAACCACAACGGAAUCGCAGACAACGCAACAAUGG |
| | | UCAGAGUAGACGUCUUCGAAAAGGGAGACAAGUACUACCUGGUCCCGAUCUACUCGUGGCAGGUCGCAAAGGGAAUCCUGCCGGACAGAGC |
| | | AGUCGUCCAGGGAAAGGACGAAGAAGACUGGCAGCUGAUCGACGACUCGUUCAACUUCAAGUUCUCGCUGCACCCGAACGACCUGGUCGAA |
| | | GUCAUCACAAAGAAGGCAAGAAUGUUCGGAUACUUCGCAUCGUGCCACAGAGGAACAGGAAACAUCAACAUCAGAAUCCACGACCUGGACC |
| | | ACAAGAUCGGAAAGAACGGAAUCCUGGAAGGAAUCGGAGUCAAGACAGCACUGUCGUUCCAGAAGUACCAGAUCGACGAACUGGGAAAGGA |
| | | AAUCAGACCGUGCAGACUGAAGAAGAGACCGCCGGUCAGAUCCGGAAAGAGAACAGCAGACGGAUCGGAAUUCGAAUCGCCGAAGAAGAAG |
| | | AGAAAGGUCGAAUGA |
| 99 | Spy- | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | R1335S | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | Cas9- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | WT | DLDNLLAQIGDQYADLFLAAKNLSDAILLSDILRVNTEITKAPLSASMIKRYDEHHQDLTLLKALVRQQLPEKYKEIFFDQSKNGYAGYID |
| | NmeCas9 | GGASQEEFYKFIKPILEKMDGTEELLVKLNREDLLRKQRTFDNGSIPHQIHLGELHAILRRQEDFYPFLKDNREKIEKILTFRIPYYVGPL |
| | fusion | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | amino | AIVDLLFKTNRKVTVKQLKEDYFKKIECFDSVEISGVEDRFNASLGTYHDLLKIIKDKDFLDNEENEDILEDIVLTLTLFEDREMIEERLK |
| | acid | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | sequence | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKSYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | SINYILGLDIGIASVGWAMVEIDEEENPIRLIDLGVRVFERAEVPKTGDSLAMARRLARSVRRLTRRRAHRLLRTRRLLKREGVLQAANFD |
| | | ENGLIKSLPNTPWQLRAAALDRKLTPLEWSAVLLHLIKHRGYLSQRKNEGETADKELGALLKGVAGNAHALQTGDFRTPAELALNKFEKES |
| | | GHIRNQRSDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSPELQDEIGTAFSLFKTDEDITGRLKDRIQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLGRLNEKGYVEIDHALPFSRTWDDSFNNKVLVLGSENQNKGNQTPYEYFNGKDNSREWQEFKARVETSRF |
| | | PRSKKQRILLQKFDEDGFKERNLNDTRYVNRFLCQFVADRMRLTGKGKKRVFASNGQITNLLRGFWGLRKVRAENDRHHALDAVVVACSTV |
| | | AMQQKITRFVRYKEMNAFDGKTIDKETGEVLHQKTHFPQPWEFFAQEVMIRVFGKPDGKPEFEEADTLEKLRTLLAEKLSSRPEAVHEYVT |
| | | PLFVSRAPNRKMSGQGHMETVKSAKRLDEGVSVLRVPLTQLKLKDLEKMVNREREPKLYEALKARLEAHKDDPAKAFAEPFYKYDKAGNRT |
| | | QQVKAVRVEQVQKTGVWVRNHNGIADNATMVRVDVFEKGDKYYLVPIYSWQVAKGILPDRAVVQGKDEEDWQLIDDSFNFKFSLHPNDLVE |
| | | VITKKARMFGYFASCHRGTGNINIRIHDLDHKIGKNGILEGIGVKTALSFQKYQIDELGKEIRPCRLKKRPPVRSGKRTADGSEFESPKKK |
| | | RKVE* |
| 100 | R1333K | AUGGACAAGAAGUACAGCAUCGGACUGGACAUCGGAACAAACAGCGUCGGAUGGGCAGUCAUCACAGACGAAUACAAGGUCCCGAGCAAGA |
| | Spy-WT | AGUUCAAGGUCCUGGGAAACACAGACAGACACAGCAUCAAGAAGAACCUGAUCGGAGCACUGCUGUUCGACAGCGGAGAAACAGCAGAAGC |
| | NmeCas9 | AACAAGACUGAAGAGAACAGCAAGAAGAAGAUACACAAGAAGAAAGAACAGAAUCUGCUACCUGCAGGAAAUCUUCAGCAACGAAAUGGCA |
| | fusion | AAGGUCGACGACAGCUUCUUCCACAGACUGGAAGAAAGCUUCCUGGUCGAAGAAGACAAGAAGCACGAAAGACACCCGAUCUUCGGAAACA |
| | ORF | UCGUCGACGAAGUCGCAUACCACGAAAAGUACCCGACAAUCUACCACCUGAGAAAGAAGCUGGUCGACAGCACAGACAAGGCAGACCUGAG |
| | | ACUGAUCUACCUGGCACUGGCACACAUGAUCAAGUUCAGAGGACACUUCCUGAUCGAAGGAGACCUGAACCCGGACAACAGCGACGUCGAC |
| | | AAGCUGUUCAUCCAGCUGGUCCAGACAUACAACCAGCUGUUCGAAGAAAACCCGAUCAACGCAAGCGGAGUCGACGCAAAGGCAAUCCUGA |
| | | GCGCAAGACUGAGCAAGAGCAGAAGACUGGAAAACCUGAUCGCACAGCUGCCGGGAGAAAAGAAGAACGGACUGUUCGGAAACCUGAUCGC |
| | | ACUGAGCCUGGGACUGACACCGAACUUCAAGAGCAACUUCGACCUGGCAGAAGACGCAAAGCUGCAGCUGAGCAAGGACACAUACGACGAC |
| | | GACCUGGACAACCUGCUGGCACAGAUCGGAGACCAGUACGCAGACCUGUUCCUGGCAGCAAAGAACCUGAGCGACGCAAUCCUGCUGAGCG |
| | | ACAUCCUGAGAGUCAACACAGAAAUCACAAAGGCACCGCUGAGCGCAAGCAUGAUCAAGAGAUACGACGAACACCACCAGGACCUGACACU |
| | | GCUGAAGGCACUGGUCAGACAGCAGCUGCCGGAAAAGUACAAGGAAAUCUUCUUCGACCAGAGCAAGAACGGAUACGCAGGAUACAUCGAC |
| | | GGAGGAGCAAGCCAGGAAGAAUUCUACAAGUUCAUCAAGCCGAUCCUGGAAAAGAUGGACGGAACAGAAGAACUGCUGGUCAAGCUGAACA |
| | | GAGAAGACCUGCUGAGAAAGCAGAGAACAUUCGACAACGGAAGCAUCCCGCACCAGAUCCACCUGGGAGAACUGCACGCAAUCCUGAGAAG |
| | | ACAGGAAGACUUCUACCCGUUCCUGAAGGACAACAGAGAAAAGAUCGAAAAGAUCCUGACAUUCAGAAUCCCGUACUACGUCGGACCGCUG |
| | | GCAAGAGGAAACAGCAGAUUCGCAUGGAUGACAAGAAAGAGCGAAGAAACAAUCACACCGUGGAACUUCGAAGAAGUCGUCGACAAGGGAG |
| | | CAAGCGCACAGAGCUUCAUCGAAAGAAUGACAAACUUCGACAAGAACCUGCCGAACGAAAAGGUCCUGCCGAAGCACAGCCUGCUGUACGA |
| | | AUACUUCACAGUCUACAACGAACUGACAAAGGUCAAGUACGUCACAGAAGGAAUGAGAAAGCCGGCAUUCCUGAGCGGAGAACAGAAGAAG |
| | | GCAAUCGUCGACCUGCUGUUCAAGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGAUCAUCAAGGACAAGGACUUCCU |
| | | GGACAACGAAGAAAACGAAGACAUCCUGGAAGACAUCGUCCUGACACUGACACUGUUCGAAGACAGAGAAAUGAUCGAAGAAAGACUGAAG |
| | | ACAUACGCACACCUGUUCGACGACAAGGUCAUGAAGCAGCUGAAGAGAAGAAGAUACACAGGAUGGGGAAGACUGAGCAGAAAGCUGAUCA |
| | | ACGGAAUCAGAGACAAGCAGAGCGGAAAGACAAUCCUGGACUUCCUGAAGAGCGACGGAUUCGCAAACAGAAACUUCAUGCAGCUGAUCCA |
| | | CGACGACAGCCUGACAUUCAAGGAAGACAUCCAGAAGGCACAGGUCAGCGGACAGGGAGACAGCCUGCACGAACACAUCGCAAACCUGGCA |
| | | GGAAGCCCGGCAAUCAAGAAGGGAAUCCUGCAGACAGUCAAGGUCGUCGACGAACUGGUCAAGGUCAUGGGAAGACACAAGCCGGAAAACA |
| | | UCGUCAUCGAAAUGGCAAGAGAAAACCAGACAACACAGAAGGGACAGAAGAACAGCAGAGAAAGAAUGAAGAGAAUCGAAGAAGGAAUCAA |
| | | GGAACUGGGAAGCCAGAUCCUGAAGGAACACCCGGUCGAAAACACACAGCUGCAGAACGAAAAGCUGUACCUGUACUACCUGCAGAACGGA |
| | | AGAGACAUGUACGUCGACCAGGAACUGGACAUCAACAGACUGAGCGACUACGACGUCGACCACAUCGUCCCGCAGAGCUUCCUGAAGGACG |
| | | ACAGCAUCGACAACAAGGUCCUGACAAGAAGCGACAAGAACAGAGGAAAGAGCGACAACGUCCCGAGCGAAGAAGUCGUCAAGAAGAUGAA |
| | | GAACUACUGGAGACAGCUGCUGAACGCAAAGCUGAUCACACAGAGAAAGUUCGACAACCUGACAAAGGCAGAGAGAGGAGGACUGAGCGAA |
| | | CUGGACAAGGCAGGAUUCAUCAAGAGACAGCUGGUCGAAACAAGACAGAUCACAAAGCACGUCGCACAGAUCCUGGACAGCAGAAUGAACA |
| | | CAAAGUACGACGAAAACGACAAGCUGAUCAGAGAAGUCAAGGUCAUCACACUGAAGAGCAAGCUGGUCAGCGACUUCAGAAAGGACUUCCA |
| | | GUUCUACAAGGUCAGAGAAAUCAACAACUACCACCACGCACACGACGCAUACCUGAACGCAGUCGUCGGAACAGCACUGAUCAAGAAGUAC |
| | | CCGAAGCUGGAAAGCGAAUUCGUCUACGGAGACUACAAGGUCUACGACGUCAGAAAGAUGAUCGCAAAGAGCGAACAGGAAAUCGGAAAGG |
| | | CAACAGCAAAGUACUUCUUCUACAGCAACAUCAUGAACUUCUUCAAGACAGAAAUCACACUGGCAAACGGAGAAAUCAGAAAGAGACCGCU |
| | | GAUCGAAACAAACGGAGAAACAGGAGAAAUCGUCUGGGACAAGGGAAGAGACUUCGCAACAGUCAGAAAGGUCCUGAGCAUGCCGCAGGUC |
| | | AACAUCGUCAAGAAGACAGAAGUCCAGACAGGAGGAUUCAGCAAGGAAAGCAUCCUGCCGAAGAGAAACAGCGACAAGCUGAUCGCAAGAA |
| | | AGAAGGACUGGGACCCGAAGAAGUACGGAGGAUUCGACAGCCCGACAGUCGCAUACAGCGUCCUGGUCGUCGCAAAGGUCGAAAAGGGAAA |
| | | GAGCAAGAAGCUGAAGAGCGUCAAGGAACUGCUGGGAAUCACAAUCAUGGAAAGAAGCAGCUUCGAAAAGAACCCGAUCGACUUCCUGGAA |
| | | GCAAAGGGAUACAAGGAAGUCAAGAAGGACCUGAUCAUCAAGCUGCCGAAGUACAGCCUGUUCGAACUGGAAAACGGAAGAAAGAGAAUGC |
| | | UGGCAAGCGCAGGAGAACUGCAGAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGACGAAAUCAUCGAACAGAUCAGC |
| | | GAAUUCAGCAAGAGAGUCAUCCUGGCAGACGCAAACCUGGACAAGGUCCUGAGCGCAUACAACAAGCACAGAGACAAGCCGAUCAGAGAAC |
| | | AGGCAGAAAACAUCAUCCACCUGUUCACACUGACAAACCUGGGAGCACCGGCAGCAUUCAAGUACUUCGACACAACAAUCGACaagAAGAG |
| | | AUACACAAGCACAAAGGAAGUCCUGGACGCAACACUGAUCCACCAGAGCAUCACAGGACUGUACGAAACAAGAAUCGACCUGAGCCAGCUG |
| | | GGAGGAGACGGCGCGCCUGAGUCUGCUACAGAAAGUGGGGGAACCUCCACAGAGUCUGAAGGAAGUGCCGGCACUUCAACCGAGAGCGAAG |
| | | GGUCCGCUGGAUCUGCAGGCUCCACAUCUACUGAGGAAGGCACCAGUACAGAGUCAGAAGGCAGCGCCGGAACUUCCACCGAGUCUGAAGG |
| | | CAGUGCCGGGACAAGUGAAUCAGCUACUGAAAGCGGCGGGACAUCAACUGAGAGCGAAGGAUCCAGUUCCGGAGCAGCAUUCAAGCCGAAC |
| | | UCGAUCAACUACAUCCUGGGACUGGACAUCGGAAUCGCAUCGGUCGGAUGGGCAAUGGUCGAAAUCGACGAAGAAGAAAACCCGAUCAGAC |
| | | UGAUCGACCUGGGAGUCAGAGUCUUCGAAAGAGCAGAAGUCCCGAAGACAGGAGACUCGCUGGCAAUGGCAAGAAGACUGGCAAGAUCGGU |
| | | CAGAAGACUGACAAGAAGAAGAGCACACAGACUGCUGAGAACAAGAAGACUGCUGAAGAGAGAAGGAGUCCUGCAGGCAGCAAACUUCGAC |
| | | GAAAACGGACUGAUCAAGUCGCUGCCGAACACACCGUGGCAGCUGAGAGCAGCAGCACUGGACAGAAAGCUGACACCGCUGGAAUGGUCGG |
| | | CAGUCCUGCUGCACCUGAUCAAGCACAGAGGAUACCUGUCGCAGAGAAAGAACGAAGGAGAAACAGCAGACAAGGAACUGGGAGCACUGCU |
| | | GAAGGGAGUCGCAGGAAACGCACACGCACUGCAGACAGGAGACUUCAGAACACCGGCAGAACUGGCACUGAACAAGUUCGAAAAGGAAUCG |
| | | GGACACAUCAGAAACCAGAGAUCGGACUACUCGCACACAUUCUCGAGAAAGGACCUGCAGGCAGAACUGAUCCUGCUGUUCGAAAAGCAGA |
| | | AGGAAUUCGGAAACCCGCACGUCUCGGGAGGACUGAAGGAAGGAAUCGAAACACUGCUGAUGACACAGAGACCGGCACUGUCGGGAGACGC |
| | | AGUCCAGAAGAUGCUGGGACACUGCACAUUCGAACCGGCAGAACCGAAGGCAGCAAAGAACACAUACACAGCAGAAAGAUUCAUCUGGCUG |
| | | ACAAAGCUGAACAACCUGAGAAUCCUGGAACAGGGAUCGGAAAGACCGCUGACAGACACAGAAAGAGCAACACUGAUGGACGAACCGUACA |
| | | GAAAGUCGAAGCUGACAUACGCACAGGCAAGAAAGCUGCUGGGACUGGAAGACACAGCAUUCUUCAAGGGACUGAGAUACGGAAAGGACAA |
| | | CGCAGAAGCAUCGACACUGAUGGAAAUGAAGGCAUACCACGCAAUCUCGAGAGCACUGGAAAAGGAAGGACUGAAGGACAAGAAGUCGCCG |
| | | CUGAACCUGUCGCCGGAACUGCAGGACGAAAUCGGAACAGCAUUCUCGCUGUUCAAGACAGACGAAGACAUCACAGGAAGACUGAAGGACA |
| | | GAAUCCAGCCGGAAAUCCUGGAAGCACUGCUGAAGCACAUCUCGUUCGACAAGUUCGUCCAGAUCUCGCUGAAGGCACUGAGAAGAAUCGU |
| | | CCCGCUGAUGGAACAGGGAAAGAGAUACGACGAAGCAUGCGCAGAAAUCUACGGAGACCACUACGGAAAGAAGAACACAGAAGAAAAGAUC |
| | | UACCUGCCGCCGAUCCCGGCAGACGAAAUCAGAAACCCGGUCGUCCUGAGAGCACUGUCGCAGGCAAGAAAGGUCAUCAACGGAGUCGUCA |
| | | GAAGAUACGGAUCGCCGGCAAGAAUCCACAUCGAAACAGCAAGAGAAGUCGGAAAGUCGUUCAAGGACAGAAAGGAAAUCGAAAAGAGACA |
| | | GGAAGAAAACAGAAAGGACAGAGAAAAGGCAGCAGCAAAGUUCAGAGAAUACUUCCCGAACUUCGUCGGAGAACCGAAGUCGAAGGACAUC |
| | | CUGAAGCUGAGACUGUACGAACAGCAGCACGGAAAGUGCCUGUACUCGGGAAAGGAAAUCAACCUGGGAAGACUGAACGAAAAGGGAUACG |
| | | UCGAAAUCGACCACGCACUGCCGUUCUCGAGAACAUGGGACGACUCGUUCAACAACAAGGUCCUGGUCCUGGGAUCGGAAAACCAGAACAA |
| | | GGGAAACCAGACACCGUACGAAUACUUCAACGGAAAGGACAACUCGAGAGAAUGGCAGGAAUUCAAGGCAAGAGUCGAAACAUCGAGAUUC |
| | | CCGAGAUCGAAGAAGCAGAGAAUCCUGCUGCAGAAGUUCGACGAAGACGGAUUCAAGGAAAGAAACCUGAACGACACAAGAUACGUCAACA |
| | | GAUUCCUGUGCCAGUUCGUCGCAGACAGAAUGAGACUGACAGGAAAGGGAAAGAAGAGAGUCUUCGCAUCGAACGGACAGAUCACAAACCU |
| | | GCUGAGAGGAUUCUGGGGACUGAGAAAGGUCAGAGCAGAAAACGACAGACACCACGCACUGGACGCAGUCGUCGUCGCAUGCUCGACAGUC |
| | | GCAAUGCAGCAGAAGAUCACAAGAUUCGUCAGAUACAAGGAAAUGAACGCAUUCGACGGAAAGACAAUCGACAAGGAAACAGGAGAAGUCC |
| | | UGCACCAGAAGACACACUUCCCGCAGCCGUGGGAAUUCUUCGCACAGGAAGUCAUGAUCAGAGUCUUCGGAAAGCCGGACGGAAAGCCGGA |
| | | AUUCGAAGAAGCAGACACACUGGAAAAGCUGAGAACACUGCUGGCAGAAAAGCUGUCGUCGAGACCGGAAGCAGUCCACGAAUACGUCACA |
| | | CCGCUGUUCGUCUCGAGAGCACCGAACAGAAAGAUGUCGGGACAGGGACACAUGGAAACAGUCAAGUCGGCAAAGAGACUGGACGAAGGAG |
| | | UCUCGGUCCUGAGAGUCCCGCUGACACAGCUGAAGCUGAAGGACCUGGAAAAGAUGGUCAACAGAGAAAGAGAACCGAAGCUGUACGAAGC |
| | | ACUGAAGGCAAGACUGGAAGCACACAAGGACGACCCGGCAAAGGCAUUCGCAGAACCGUUCUACAAGUACGACAAGGCAGGAAACAGAACA |
| | | CAGCAGGUCAAGGCAGUCAGAGUCGAACAGGUCCAGAAGACAGGAGUCUGGGUCAGAAACCACAACGGAAUCGCAGACAACGCAACAAUGG |
| | | UCAGAGUAGACGUCUUCGAAAAGGGAGACAAGUACUACCUGGUCCCGAUCUACUCGUGGCAGGUCGCAAAGGGAAUCCUGCCGGACAGAGC |
| | | AGUCGUCCAGGGAAAGGACGAAGAAGACUGGCAGCUGAUCGACGACUCGUUCAACUUCAAGUUCUCGCUGCACCCGAACGACCUGGUCGAA |
| | | GUCAUCACAAAGAAGGCAAGAAUGUUCGGAUACUUCGCAUCGUGCCACAGAGGAACAGGAAACAUCAACAUCAGAAUCCACGACCUGGACC |
| | | ACAAGAUCGGAAAGAACGGAAUCCUGGAAGGAAUCGGAGUCAAGACAGCACUGUCGUUCCAGAAGUACCAGAUCGACGAACUGGGAAAGGA |
| | | AAUCAGACCGUGCAGACUGAAGAAGAGACCGCCGGUCAGAUCCGGAAAGAGAACAGCAGACGGAUCGGAAUUCGAAUCGCCGAAGAAGAAG |
| | | AGAAAGGUCGAAUGA |
| | | MDKKYSIGLDIGTNSVGWAVITDEYKVPSKKFKVLGNTDRHSIKKNLIGALLFDSGETAEATRLKRTARRRYTRRKNRICYLQEIFSNEMA |
| 101 | R1333K | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | Spy- | KLFIQLVQTYNQLFEENPINASGVDAKAILSARLSKSRRLENLIAQLPGEKKNGLFGNLIALSLGLTPNFKSNFDLAEDAKLQLSKDTYDD |
| | WT | DLDNLLAQIGDQYADLFLAAKNLSDAILLSDILRVNTEITKAPLSASMIKRYDEHHQDLTLLKALVRQQLPEKYKEIFFDQSKNGYAGYID |
| | NmeCas9 | GGASQEEFYKFIKPILEKMDGTEELLVKLNREDLLRKQRTFDNGSIPHQIHLGELHAILRRQEDFYPFLKDNREKIEKILTFRIPYYVGPL |
| | fusion | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | amino | AIVDLLFKTNRKVTVKQLKEDYFKKIECFDSVEISGVEDRFNASLGTYHDLLKIIKDKDFLDNEENEDILEDIVLTLTLFEDREMIEERLK |
| | acid | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | sequence | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGDGAPESATESGGTSTESEGSAGTSTESEGSAGSAGSTSTEEGTSTESEGSAGTSTESEGSAGTSESATESGGTSTESEGSSSGAAFKPN |
| | | SINYILGLDIGIASVGWAMVEIDEEENPIRLIDLGVRVFERAEVPKTGDSLAMARRLARSVRRLTRRRAHRLLRTRRLLKREGVLQAANFD |
| | | ENGLIKSLPNTPWQLRAAALDRKLTPLEWSAVLLHLIKHRGYLSQRKNEGETADKELGALLKGVAGNAHALQTGDFRTPAELALNKFEKES |
| | | GHIRNQRSDYSHTFSRKDLQAELILLFEKQKEFGNPHVSGGLKEGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLEDTAFFKGLRYGKDNAEASTLMEMKAYHAISRALEKEGLKDKKSP |
| | | LNLSPELQDEIGTAFSLFKTDEDITGRLKDRIQPEILEALLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKDRKEIEKRQEENRKDREKAAAKFREYFPNFVGEPKSKDI |
| | | LKLRLYEQQHGKCLYSGKEINLGRLNEKGYVEIDHALPFSRTWDDSFNNKVLVLGSENQNKGNQTPYEYFNGKDNSREWQEFKARVETSRF |
| | | PRSKKQRILLQKFDEDGFKERNLNDTRYVNRFLCQFVADRMRLTGKGKKRVFASNGQITNLLRGFWGLRKVRAENDRHHALDAVVVACSTV |
| | | AMQQKITRFVRYKEMNAFDGKTIDKETGEVLHQKTHFPQPWEFFAQEVMIRVFGKPDGKPEFEEADTLEKLRTLLAEKLSSRPEAVHEYVT |
| | | PLFVSRAPNRKMSGQGHMETVKSAKRLDEGVSVLRVPLTQLKLKDLEKMVNREREPKLYEALKARLEAHKDDPAKAFAEPFYKYDKAGNRT |
| | | QQVKAVRVEQVQKTGVWVRNHNGIADNATMVRVDVFEKGDKYYLVPIYSWQVAKGILPDRAVVQGKDEEDWQLIDDSFNFKFSLHPNDLVE |
| | | VITKKARMFGYFASCHRGTGNINIRIHDLDHKIGKNGILEGIGVKTALSFQKYQIDELGKEIRPCRLKKRPPVRSGKRTADGSEFESPKKK |
| | | RKVE* |
| 102 | WT | AUGGACAAGAAGUACAGCAUCGGACUGGACAUCGGAACAAACAGCGUCGGAUGGGCAGUCAUCACAGACGAAUACAA |
| | SpyCas9- | GGUCCCGAGCAAGAAGUUCAAGGUCCUGGGAAACACAGACAGACACAGCAUCAAGAAGAACCUGAUCGGAGCACUGC |
| | WT | UGUUCGACAGCGGAGAAACAGCAGAAGCAACAAGACUGAAGAGAACAGCAAGAAGAAGAUACACAAGAAGAAAGAAC |
| | NmeCas9 | AGAAUCUGCUACCUGCAGGAAAUCUUCAGCAACGAAAUGGCAAAGGUCGACGACAGCUUCUUCCACAGACUGGAAGA |
| | fusion | AAGCUUCCUGGUCGAAGAAGACAAGAAGCACGAAAGACACCCGAUCUUCGGAAACAUCGUCGACGAAGUCGCAUACC |
| | ORF | ACGAAAAGUACCCGACAAUCUACCACCUGAGAAAGAAGCUGGUCGACAGCACAGACAAGGCAGACCUGAGACUGAUC |
| | | UACCUGGCACUGGCACACAUGAUCAAGUUCAGAGGACACUUCCUGAUCGAAGGAGACCUGAACCCGGACAACAGCGAC |
| | | GUCGACAAGCUGUUCAUCCAGCUGGUCCAGACAUACAACCAGCUGUUCGAAGAAAACCCGAUCAACGCAAGCGGAGUC |
| | | GACGCAAAGGCAAUCCUGAGCGCAAGACUGAGCAAGAGCAGAAGACUGGAAAACCUGAUCGCACAGCUGCCGGGAGA |
| | | AAAGAAGAACGGACUGUUCGGAAACCUGAUCGCACUGAGCCUGGGACUGACACCGAACUUCAAGAGCAACUUCGACC |
| | | UGGCAGAAGACGCAAAGCUGCAGCUGAGCAAGGACACAUACGACGACGACCUGGACAACCUGCUGGCACAGAUCGGA |
| | | GACCAGUACGCAGACCUGUUCCUGGCAGCAAAGAACCUGAGCGACGCAAUCCUGCUGAGCGACAUCCUGAGAGUCAAC |
| | | ACAGAAAUCACAAAGGCACCGCUGAGCGCAAGCAUGAUCAAGAGAUACGACGAACACCACCAGGACCUGACACUGCUG |
| | | AAGGCACUGGUCAGACAGCAGCUGCCGGAAAAGUACAAGGAAAUCUUCUUCGACCAGAGCAAGAACGGAUACGCAGG |
| | | AUACAUCGACGGAGGAGCAAGCCAGGAAGAAUUCUACAAGUUCAUCAAGCCGAUCCUGGAAAAGAUGGACGGAACAG |
| | | AAGAACUGCUGGUCAAGCUGAACAGAGAAGACCUGCUGAGAAAGCAGAGAACAUUCGACAACGGAAGCAUCCCGCAC |
| | | CAGAUCCACCUGGGAGAACUGCACGCAAUCCUGAGAAGACAGGAAGACUUCUACCCGUUCCUGAAGGACAACAGAGA |
| | | AAAGAUCGAAAAGAUCCUGACAUUCAGAAUCCCGUACUACGUCGGACCGCUGGCAAGAGGAAACAGCAGAUUCGCAU |
| | | GGAUGACAAGAAAGAGCGAAGAAACAAUCACACCGUGGAACUUCGAAGAAGUCGUCGACAAGGGAGCAAGCGCACAG |
| | | AGCUUCAUCGAAAGAAUGACAAACUUCGACAAGAACCUGCCGAACGAAAAGGUCCUGCCGAAGCACAGCCUGCUGUAC |
| | | GAAUACUUCACAGUCUACAACGAACUGACAAAGGUCAAGUACGUCACAGAAGGAAUGAGAAAGCCGGCAUUCCUGAG |
| | | CGGAGAACAGAAGAAGGCAAUCGUCGACCUGCUGUUCAAGACAAACAGAAAGGUCACAGUCAAGCAGCUGAAGGAAG |
| | | ACUACUUCAAGAAGAUCGAAUGCUUCGACAGCGUCGAAAUCAGCGGAGUCGAAGACAGAUUCAACGCAAGCCUGGGA |
| | | ACAUACCACGACCUGCUGAAGAUCAUCAAGGACAAGGACUUCCUGGACAACGAAGAAAACGAAGACAUCCUGGAAGA |
| | | CAUCGUCCUGACACUGACACUGUUCGAAGACAGAGAAAUGAUCGAAGAAAGACUGAAGACAUACGCACACCUGUUCG |
| | | ACGACAAGGUCAUGAAGCAGCUGAAGAGAAGAAGAUACACAGGAUGGGGAAGACUGAGCAGAAAGCUGAUCAACGGA |
| | | AUCAGAGACAAGCAGAGCGGAAAGACAAUCCUGGACUUCCUGAAGAGCGACGGAUUCGCAAACAGAAACUUCAUGCA |
| | | GCUGAUCCACGACGACAGCCUGACAUUCAAGGAAGACAUCCAGAAGGCACAGGUCAGCGGACAGGGAGACAGCCUGCA |
| | | CGAACACAUCGCAAACCUGGCAGGAAGCCCGGCAAUCAAGAAGGGAAUCCUGCAGACAGUCAAGGUCGUCGACGAACU |
| | | GGUCAAGGUCAUGGGAAGACACAAGCCGGAAAACAUCGUCAUCGAAAUGGCAAGAGAAAACCAGACAACACAGAAGG |
| | | GACAGAAGAACAGCAGAGAAAGAAUGAAGAGAAUCGAAGAAGGAAUCAAGGAACUGGGAAGCCAGAUCCUGAAGGAA |
| | | CACCCGGUCGAAAACACACAGCUGCAGAACGAAAAGCUGUACCUGUACUACCUGCAGAACGGAAGAGACAUGUACGUC |
| | | GACCAGGAACUGGACAUCAACAGACUGAGCGACUACGACGUCGACCACAUCGUCCCGCAGAGCUUCCUGAAGGACGAC |
| | | AGCAUCGACAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAACCUGACAA |
| | | AGGCAGAGAGAGGAGGACUGAGCGAACUGGACAAGGCAGGAUUCAUCAAGAGACAGCUGGUCGAAACAAGACAGAUC |
| | | ACAAAGCACGUCGCACAGAUCCUGGACAGCAGAAUGAACACAAAGUACGACGAAAACGACAAGCUGAUCAGAGAAGU |
| | | CAAGGUCAUCACACUGAAGAGCAAGCUGGUCAGCGACUUCAGAAAGGACUUCCAGUUCUACAAGGUCAGAGAAAUCA |
| | | ACAACUACCACCACGCACACGACGCAUACCUGAACGCAGUCGUCGGAACAGCACUGAUCAAGAAGUACCCGAAGCUGG |
| | | AAAGCGAAUUCGUCUACGGAGACUACAAGGUCUACGACGUCAGAAAGAUGAUCGCAAAGAGCGAACAGGAAAUCGGA |
| | | AAGGCAACAGCAAAGUACUUCUUCUACAGCAACAUCAUGAACUUCUUCAAGACAGAAAUCACACUGGCAAACGGAGA |
| | | AAUCAGAAAGAGACCGCUGAUCGAAACAAACGGAGAAACAGGAGAAAUCGUCUGGGACAAGGGAAGAGACUUCGCAA |
| | | CAGUCAGAAAGGUCCUGAGCAUGCCGCAGGUCAACAUCGUCAAGAAGACAGAAGUCCAGACAGGAGGAUUCAGCAAG |
| | | GAAAGCAUCCUGCCGAAGAGAAACAGCGACAAGCUGAUCGCAAGAAAGAAGGACUGGGACCCGAAGAAGUACGGAGG |
| | | AUUCGACAGCCCGACAGUCGCAUACAGCGUCCUGGUCGUCGCAAAGGUCGAAAAGGGAAAGAGCAAGAAGCUGAAGA |
| | | GCGUCAAGGAACUGCUGGGAAUCACAAUCAUGGAAAGAAGCAGCUUCGAAAAGAACCCGAUCGACUUCCUGGAAGCA |
| | | AAGGGAUACAAGGAAGUCAAGAAGGACCUGAUCAUCAAGCUGCCGAAGUACAGCCUGUUCGAACUGGAAAACGGAAG |
| | | AAAGAGAAUGCUGGCAAGCGCAGGAGAACUGCAGAAGGGAAACGAACUGGCACUGCCGAGCAAGUACGUCAACUUCC |
| | | UGUACCUGGCAAGCCACUACGAAAAGCUGAAGGGAAGCCCGGAAGACAACGAACAGAAGCAGCUGUUCGUCGAACAG |
| | | CACAAGCACUACCUGGACGAAAUCAUCGAACAGAUCAGCGAAUUCAGCAAGAGAGUCAUCCUGGCAGACGCAAACCUG |
| | | GACAAGGUCCUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUACACAAGCACA |
| | | AAGGAAGUCCUGGACGCAACACUGAUCCACCAGAGCAUCACAGGACUGUACGAAACAAGAAUCGACCUGAGCCAGCUG |
| | | GGAGGAGACGGCGCGCCUGAGUCUGCUACAGAAAGUGGGGGAACCUCCACAGAGUCUGAAGGAAGUGCCGGCACUUC |
| | | AACCGAGAGCGAAGGGUCCGCUGGAUCUGCAGGCUCCACAUCUACUGAGGAAGGCACCAGUACAGAGUCAGAAGGCA |
| | | GCGCCGGAACUUCCACCGAGUCUGAAGGCAGUGCCGGGACAAGUGAAUCAGCUACUGAAAGCGGCGGGACAUCAACU |
| | | GAGAGCGAAGGAUCCAGUUCCGGAGCAGCAUUCAAGCCGAACUCGAUCAACUACAUCCUGGGACUGGACAUCGGAAU |
| | | CGCAUCGGUCGGAUGGGCAAUGGUCGAAAUCGACGAAGAAGAAAACCCGAUCAGACUGAUCGACCUGGGAGUCAGAG |
| | | UCUUCGAAAGAGCAGAAGUCCCGAAGACAGGAGACUCGCUGGCAAUGGCAAGAAGACUGGCAAGAUCGGUCAGAAGA |
| | | CUGACAAGAAGAAGAGCACACAGACUGCUGAGAACAAGAAGACUGCUGAAGAGAGAAGGAGUCCUGCAGGCAGCAAA |
| | | CUUCGACGAAAACGGACUGAUCAAGUCGCUGCCGAACACACCGUGGCAGCUGAGAGCAGCAGCACUGGACAGAAAGCU |
| | | GACACCGCUGGAAUGGUCGGCAGUCCUGCUGCACCUGAUCAAGCACAGAGGAUACCUGUCGCAGAGAAAGAACGAAG |
| | | GAGAAACAGCAGACAAGGAACUGGGAGCACUGCUGAAGGGAGUCGCAGGAAACGCACACGCACUGCAGACAGGAGAC |
| | | UUCAGAACACCGGCAGAACUGGCACUGAACAAGUUCGAAAAGGAAUCGGGACACAUCAGAAACCAGAGAUCGGACUA |
| | | CUCGCACACAUUCUCGAGAAAGGACCUGCAGGCAGAACUGAUCCUGCUGUUCGAAAAGCAGAAGGAAUUCGGAAACC |
| | | CGCACGUCUCGGGAGGACUGAAGGAAGGAAUCGAAACACUGCUGAUGACACAGAGACCGGCACUGUCGGGAGACGCA |
| | | GUCCAGAAGAUGCUGGGACACUGCACAUUCGAACCGGCAGAACCGAAGGCAGCAAAGAACACAUACACAGCAGAAAG |
| | | AUUCAUCUGGCUGACAAAGCUGAACAACCUGAGAAUCCUGGAACAGGGAUCGGAAAGACCGCUGACAGACACAGAAA |
| | | GAGCAACACUGAUGGACGAACCGUACAGAAAGUCGAAGCUGACAUACGCACAGGCAAGAAAGCUGCUGGGACUGGAA |
| | | GACACAGCAUUCUUCAAGGGACUGAGAUACGGAAAGGACAACGCAGAAGCAUCGACACUGAUGGAAAUGAAGGCAUA |
| | | CCACGCAAUCUCGAGAGCACUGGAAAAGGAAGGACUGAAGGACAAGAAGUCGCCGCUGAACCUGUCGCCGGAACUGC |
| | | AGGACGAAAUCGGAACAGCAUUCUCGCUGUUCAAGACAGACGAAGACAUCACAGGAAGACUGAAGGACAGAAUCCAG |
| | | CCGGAAAUCCUGGAAGCACUGCUGAAGCACAUCUCGUUCGACAAGUUCGUCCAGAUCUCGCUGAAGGCACUGAGAAG |
| | | AAUCGUCCCGCUGAUGGAACAGGGAAAGAGAUACGACGAAGCAUGCGCAGAAAUCUACGGAGACCACUACGGAAAGA |
| | | AGAACACAGAAGAAAAGAUCUACCUGCCGCCGAUCCCGGCAGACGAAAUCAGAAACCCGGUCGUCCUGAGAGCACUGU |
| | | CGCAGGCAAGAAAGGUCAUCAACGGAGUCGUCAGAAGAUACGGAUCGCCGGCAAGAAUCCACAUCGAAACAGCAAGA |
| | | GAAGUCGGAAAGUCGUUCAAGGACAGAAAGGAAAUCGAAAAGAGACAGGAAGAAAACAGAAAGGACAGAGAAAAGGC |
| | | AGCAGCAAAGUUCAGAGAAUACUUCCCGAACUUCGUCGGAGAACCGAAGUCGAAGGACAUCCUGAAGCUGAGACUGU |
| | | ACGAACAGCAGCACGGAAAGUGCCUGUACUCGGGAAAGGAAAUCAACCUGGGAAGACUGAACGAAAAGGGAUACGUC |
| | | GAAAUCGACCACGCACUGCCGUUCUCGAGAACAUGGGACGACUCGUUCAACAACAAGGUCCUGGUCCUGGGAUCGGAA |
| | | AACCAGAACAAGGGAAACCAGACACCGUACGAAUACUUCAACGGAAAGGACAACUCGAGAGAAUGGCAGGAAUUCAA |
| | | GGCAAGAGUCGAAACAUCGAGAUUCCCGAGAUCGAAGAAGCAGAGAAUCCUGCUGCAGAAGUUCGACGAAGACGGAU |
| | | UCAAGGAAAGAAACCUGAACGACACAAGAUACGUCAACAGAUUCCUGUGCCAGUUCGUCGCAGACAGAAUGAGACUG |
| | | ACAGGAAAGGGAAAGAAGAGAGUCUUCGCAUCGAACGGACAGAUCACAAACCUGCUGAGAGGAUUCUGGGGACUGAG |
| | | AAAGGUCAGAGCAGAAAACGACAGACACCACGCACUGGACGCAGUCGUCGUCGCAUGCUCGACAGUCGCAAUGCAGCA |
| | | GAAGAUCACAAGAUUCGUCAGAUACAAGGAAAUGAACGCAUUCGACGGAAAGACAAUCGACAAGGAAACAGGAGAAG |
| | | UCCUGCACCAGAAGACACACUUCCCGCAGCCGUGGGAAUUCUUCGCACAGGAAGUCAUGAUCAGAGUCUUCGGAAAGC |
| | | CGGACGGAAAGCCGGAAUUCGAAGAAGCAGACACACUGGAAAAGCUGAGAACACUGCUGGCAGAAAAGCUGUCGUCG |
| | | AGACCGGAAGCAGUCCACGAAUACGUCACACCGCUGUUCGUCUCGAGAGCACCGAACAGAAAGAUGUCGGGACAGGG |
| | | ACACAUGGAAACAGUCAAGUCGGCAAAGAGACUGGACGAAGGAGUCUCGGUCCUGAGAGUCCCGCUGACACAGCUGA |
| | | AGCUGAAGGACCUGGAAAAGAUGGUCAACAGAGAAAGAGAACCGAAGCUGUACGAAGCACUGAAGGCAAGACUGGAA |
| | | GCACACAAGGACGACCCGGCAAAGGCAUUCGCAGAACCGUUCUACAAGUACGACAAGGCAGGAAACAGAACACAGCAG |
| | | GUCAAGGCAGUCAGAGUCGAACAGGUCCAGAAGACAGGAGUCUGGGUCAGAAACCACAACGGAAUCGCAGACAACGC |
| | | AACAAUGGUCAGAGUAGACGUCUUCGAAAAGGGAGACAAGUACUACCUGGUCCCGAUCUACUCGUGGCAGGUCGCAA |
| | | AGGGAAUCCUGCCGGACAGAGCAGUCGUCCAGGGAAAGGACGAAGAAGACUGGCAGCUGAUCGACGACUCGUUCAAC |
| | | UUCAAGUUCUCGCUGCACCCGAACGACCUGGUCGAAGUCAUCACAAAGAAGGCAAGAAUGUUCGGAUACUUCGCAUC |
| | | GUGCCACAGAGGAACAGGAAACAUCAACAUCAGAAUCCACGACCUGGACCACAAGAUCGGAAAGAACGGAAUCCUGG |
| | | AAGGAAUCGGAGUCAAGACAGCACUGUCGUUCCAGAAGUACCAGAUCGACGAACUGGGAAAGGAAAUCAGACCGUGC |
| | | AGACUGAAGAAGAGACCGCCGGUCAGAUCCGGAAAGAGAACAGCAGACGGAUCGGAAUUCGAAUCGCCGAAGAAGAA |
| | | GAGAAAGGUCGAAUGA |
| 103 | WT | MDKKYSIGLDIGTNSVGWAVITDEYKVPSKKFKVLGNTDRHSIKKNLIGALLFDSGETAEATRLKRTARRRYTRRKNRICYLQEI |
| | SpyCas9- | FSNEMAKVDDSFFHRLEESFLVEEDKKHERHPIFGNIVDEVAYHEKYPTIYHLRKKLVDSTDKADLRLIYLALAHMIKFRGHFLI |
| | WT | EGDLNPDNSDVDKLFIQLVQTYNQLFEENPINASGVDAKAILSARLSKSRRLENLIAQLPGEKKNGLFGNLIALSLGLTPNFKSNF |
| | NmeCas9 | DLAEDAKLQLSKDTYDDDLDNLLAQIGDQYADLFLAAKNLSDAILLSDILRVNTEITKAPLSASMIKRYDEHHQDLTLLKALVR |
| | fusion | QQLPEKYKEIFFDQSKNGYAGYIDGGASQEEFYKFIKPILEKMDGTEELLVKLNREDLLRKQRTFDNGSIPHQIHLGELHAILRRQ |
| | ORF | EDFYPFLKDNREKIEKILTFRIPYYVGPLARGNSRFAWMTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLFDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVKVVDELVKVMGRHKPENIVIEMA |
| | | RENQTTQKGQKNSRERMKRIEEGIKELGSQILKEHPVENTQLQNEKLYLYYLQNGRDMYVDQELDINRLSDYDVDHIVPQSFL |
| | | KDDSIDNKVLTRSDKNRGKSDNVPSEEVVKKMKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHHAHDAYLNAVVGTALIKKYPKLESEFVYGDY |
| | | KVYDVRKMIAKSEQEIGKATAKYFFYSNIMNFFKTEITLANGEIRKRPLIETNGETGEIVWDKGRDFATVRKVLSMPQVNIVKKT |
| | | EVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYL |
| | | DEIIEQISEFSKRVILADANLDKVLSAYNKHRDKPIREQAENIIHLFTLTNLGAPAAFKYFDTTIDRKRYTSTKEVLDATLIHQSITG |
| | | LYETRIDLSQLGGDGAPESATESGGTSTESEGSAGTSTESEGSAGSAGSTSTEEGTSTESEGSAGTSTESEGSAGTSESATESGGTS |
| | | TESEGSSSGAAFKPNSINYILGLDIGIASVGWAMVEIDEEENPIRLIDLGVRVFERAEVPKTGDSLAMARRLARSVRRLTRRRAHR |
| | | LLRTRRLLKREGVLQAANFDENGLIKSLPNTPWQLRAAALDRKLTPLEWSAVLLHLIKHRGYLSQRKNEGETADKELGALLKG |
| | | VAGNAHALQTGDFRTPAELALNKFEKESGHIRNQRSDYSHTFSRKDLQAELILLFEKQKEFGNPHVSGGLKEGIETLLMTQRPA |
| | | LSGDAVQKMLGHCTFEPAEPKAAKNTYTAERFIWLTKLNNLRILEQGSERPLTDTERATLMDEPYRKSKLTYAQARKLLGLED |
| | | TAFFKGLRYGKDNAEASTLMEMKAYHAISRALEKEGLKDKKSPLNLSPELQDEIGTAFSLFKTDEDITGRLKDRIQPEILEALLK |
| | | HISFDKFVQISLKALRRIVPLMEQGKRYDEACAEIYGDHYGKKNTEEKIYLPPIPADEIRNPVVLRALSQARKVINGVVRRYGSP |
| | | ARIHIETAREVGKSFKDRKEIEKRQEENRKDREKAAAKFREYFPNFVGEPKSKDILKLRLYEQQHGKCLYSGKEINLGRLNEKG |
| | | YVEIDHALPFSRTWDDSFNNKVLVLGSENQNKGNQTPYEYFNGKDNSREWQEFKARVETSRFPRSKKQRILLQKFDEDGFKER |
| | | NLNDTRYVNRFLCQFVADRMRLTGKGKKRVFASNGQITNLLRGFWGLRKVRAENDRHHALDAVVVACSTVAMQQKITRFVR |
| | | YKEMNAFDGKTIDKETGEVLHQKTHFPQPWEFFAQEVMIRVFGKPDGKPEFEEADTLEKLRTLLAEKLSSRPEAVHEYVTPLFV |
| | | SRAPNRKMSGQGHMETVKSAKRLDEGVSVLRVPLTQLKLKDLEKMVNREREPKLYEALKARLEAHKDDPAKAFAEPFYKYD |
| | | KAGNRTQQVKAVRVEQVQKTGVWVRNHNGIADNATMVRVDVFEKGDKYYLVPIYSWQVAKGILPDRAVVQGKDEEDWQLI |
| | | DDSFNFKFSLHPNDLVEVITKKARMFGYFASCHRGTGNINIRIHDLDHKIGKNGILEGIGVKTALSFQKYQIDELGKEIRPCRLKK |
| | | RPPVRSGKRTADGSEFESPKKKRKVE* |
| 104 | Spy | GGCGACAAGAAGUACUCCAUCGGCCUGGACAUCGGCACCAACUCCGUGGGCUGGGCCGUGAUCACCGACGAGUACAAGGUGCCCUCCAAGA |
| | Cas9 | AGUUCAAGGUGCUGGGCAACACCGACCGGCACUCCAUCAAGAAGAACCUGAUCGGCGCCCUGCUGUUCGACUCCGGCGAGACCGCCGAGGC |
| | R133K | CACCCGGCUGAAGCGGACCGCCCGGCGGCGGUACACCCGGCGGAAGAACCGGAUCUGCUACCUGCAGGAGAUCUUCUCCAACGAGAUGGCC |
| | nucleic | AAGGUGGACGACUCCUUCUUCCACCGGCUGGAGGAGUCCUUCCUGGUGGAGGAGGACAAGAAGCACGAGCGGCACCCCAUCUUCGGCAACA |
| | acid | UCGUGGACGAGGUGGCCUACCACGAGAAGUACCCCACCAUCUACCACCUGCGGAAGAAGCUGGUGGACUCCACCGACAAGGCCGACCUGCG |
| | coding | GCUGAUCUACCUGGCCCUGGCCCACAUGAUCAAGUUCCGGGGCCACUUCCUGAUCGAGGGCGACCUGAACCCCGACAACUCCGACGUGGAC |
| | sequence | AAGCUGUUCAUCCAGCUGGUGCAGACCUACAACCAGCUGUUCGAGGAGAACCCCAUCAACGCCUCCGGCGUGGACGCCAAGGCCAUCCUGU |
| | without | CCGCCCGGCUGUCCAAGUCCCGGCGGCUGGAGAACCUGAUCGCCCAGCUGCCCGGCGAGAAGAAGAACGGCCUGUUCGGCAACCUGAUCGC |
| | start or | CCUGUCCCUGGGCCUGACCCCCAACUUCAAGUCCAACUUCGACCUGGCCGAGGACGCCAAGCUGCAGCUGUCCAAGGACACCUACGACGAC |
| | stop | GACCUGGACAACCUGCUGGCCCAGAUCGGCGACCAGUACGCCGACCUGUUCCUGGCCGCCAAGAACCUGUCCGACGCCAUCCUGCUGUCCG |
| | codons | ACAUCCUGCGGGUGAACACCGAGAUCACCAAGGCCCCCCUGUCCGCCUCCAUGAUCAAGCGGUACGACGAGCACCACCAGGACCUGACCCU |
| | suitable | GCUGAAGGCCCUGGUGCGGCAGCAGCUGCCCGAGAAGUACAAGGAGAUCUUCUUCGACCAGUCCAAGAACGGCUACGCCGGCUACAUCGAC |
| | for | GGCGGCGCCUCCCAGGAGGAGUUCUACAAGUUCAUCAAGCCCAUCCUGGAGAAGAUGGACGGCACCGAGGAGCUGCUGGUGAAGCUGAACC |
| | fusion | GGGAGGACCUGCUGCGGAAGCAGCGGACCUUCGACAACGGCUCCAUCCCCCACCAGAUCCACCUGGGCGAGCUGCACGCCAUCCUGCGGCG |
| | constructs | GCAGGAGGACUUCUACCCCUUCCUGAAGGACAACCGGGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCACCCCCUGGAACUUCGAGGAGGUGGUGGACAAGGGCG |
| | | CCUCCGCCCAGUCCUUCAUCGAGCGGAUGACCAACUUCGACAAGAACCUGCCCAACGAGAAGGUGCUGCCCAAGCACUCCCUGCUGUACGA |
| | | GUACUUCACCGUGUACAACGAGCUGACCAAGgugaaguacgugaccgagggcaugcggaagcccgccuuccuguccggcgagcagaagaag |
| | | gccaucguggaccugcuguucaagaccaaccggaaggugaccgugaagcagcugaaggaggacuacuucaagaagaucgagugcuucgacu |
| | | ccguggagaucuccggcguggaggaccgguucaacgccucccugggcaccuaccacgaccugcugaagaucaucaaggacaaggacuuccu |
| | | ggacaacgaggagaacgaggacauccuggaggacaucgugcugacccugacccuguucgaggaccgggagaugaucgaggagcggcugaag |
| | | accuacgcccaccuguucgacgacaaggugaugaagcagcugaagcggcggcgguacaccggcuggggccggcugucccggaagcugauca |
| | | acggcauccgggacaagcaguccggcaagaccauccuggacuuccugaaguccgacggcuucgccaaccggaacuucaugcagcugaucca |
| | | cgacgacucccugaccuucaaggaggacauccagaaggcccagguguccggccagggcgacucccugcacgagcacaucgccaaccuggcc |
| | | ggcucccccgccaucaagaagggcauccugcagaccgugaaggugguggacgagcuggugaaggugaugggccggcacaagcccgagaaca |
| | | ucgugaucgagauggcccgggagaaccagaccacccagaagggccagaagaacucccgggagcggaugaagcggaucgaggagggcaucaa |
| | | ggagcugggcucccagauccugaaggagcaccccguggagaacacccagcugcagaacgagaagcuguaccuguacuaccugcagaacggc |
| | | cgggacauguacguggaccaggagcuggacaucaaccggcuguccgacuacgacguggaccacaucgugccccaguccuuccugaaggacg |
| | | acuccaucgacaacaaggugcugacccgguccgacaagaaccggggcaaguccgacaacgugcccuccgaggagguggugaagaagaugaa |
| | | gaacuacuggcggcagcugcugaacgccaagcugaucacccagcggaaguucgacaaccugaccaaggccgagcggCggggccuguccgag |
| | | cuggacaaggccggcuucaucaagcggcagcugguggagacccggcagaucaccaagcacguggcccagauccuggacucccggaugaaca |
| | | ccaaguacgacgagaacgacaagcugauccgggaggugaaggugaucacccugaaguccaagcugguguccgacuuccggaaggacuucca |
| | | guucuacaaggugcgggagaucaacaacuaccaccacgcccacgacgccuaccugaacgccguggugggcaccgcccugaucaagaaguac |
| | | cccaagcuggaguccgaguucguguacggcgacuacaagguguacgacgugcggaagaugaucgccaaguccgagcaggagaucggcaagg |
| | | ccaccgccaaguacuucuucuacuccaacaucaugaacuucuucaagaccgagaucacccuggccaacggcgagauccggaagcggccccu |
| | | gaucgagaccaacggcgagaccggcgagaucgugugggacaagggccgggacuucgccaccgugcggaaggugcuguccaugccccaggug |
| | | aacaucgugaagaagaccgaggugcagaccggcggcuucuccaaggaguccauccugcccaagcggaacuccgacaagcugaucgcccgga |
| | | agaaggacugggaccccaagaaguacggcggcuucgacucccccaccguggccuacuccgugcuggugguggccaagguggagaagggcaa |
| | | guccaagaagcugaaguccgugaaggagcugcugggcaucaccaucauggagcgguccuccuucgagaagaaccccaucgacuuccuggag |
| | | gccaagggcuacaaggaggugaagaaggaccugaucaucaagcugcccaaguacucccuguucgagcuggagaacggccggaagcggaugc |
| | | uggccuccgccggcgagcugcagaagggcaacgagcuggcccugcccuccaaguacgugaacuuccuguaccuggccucccacuacgagaa |
| | | gcugaagggcucccccgaggacaacgagcagaagcagcuguucguggagcagcacaagcacuaccuggacgagaucaucgagcagaucucc |
| | | gaguucuccaagcgggugauccuggccgacgccaaccuggacaaggugcuguccgccuacaacaagcaccgggacaagcccauccgggagc |
| | | aggccgagaacaucauccaccuguucacccugaccaaccugggcgcccccgccgccuucaaguacuucgacaccaccaucgacAAGaagcg |
| | | guacaccuccaccaaggaggugcuggacgccacccugauccaccaguccaucaccggccuguacgagacccggaucgaccugucccagcug |
| | | ggcggcgac |
| 105 | Amino | GDKKYSIGLDIGTNSVGWAVITDEYKVPSKKFKVLGNTDRHSIKKNLIGALLFDSGETAEATRLKRTARRRYTRRKNRICYLQEIFSNEMA |
| | acid | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | sequence | KLFIQLVQTYNQLFEENPINASGVDAKAILSARLSKSRRLENLIAQLPGEKKNGLFGNLIALSLGLTPNFKSNFDLAEDAKLQLSKDTYDD |
| | for | DLDNLLAQIGDQYADLFLAAKNLSDAILLSDILRVNTEITKAPLSASMIKRYDEHHQDLTLLKALVRQQLPEKYKEIFFDQSKNGYAGYID |
| | SpyCas9 | GGASQEEFYKFIKPILEKMDGTEELLVKLNREDLLRKQRTFDNGSIPHQIHLGELHAILRRQEDFYPFLKDNREKIEKILTFRIPYYVGPL |
| | R1333K | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | suitable | AIVDLLFKTNRKVTVKQLKEDYFKKIECFDSVEISGVEDRFNASLGTYHDLLKIIKDKDFLDNEENEDILEDIVLTLTLFEDREMIEERLK |
| | for | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | fusion | GSPAIKKGILQTVKVVDELVKVMGRHKPENIVIEMARENQTTQKGQKNSRERMKRIEEGIKELGSQILKEHPVENTQLQNEKLYLYYLQNG |
| | constructs | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDKKRYTSTKEVLDATLIHQSITGLYETRIDLSQL |
| | | GGD |
| 106 | WT | AUGGCAGCAUUCAAGCCGAACUCGAUCAACUACAUCCUGGGACUGGACAUCGGAAUCGCAUCGGUCGGAUGGGCAAU |
| | Nme1 | GGUCGAAAUCGACGAAGAAGAAAACCCGAUCAGACUGAUCGACCUGGGAGUCAGAGUCUUCGAAAGAGCAGAAGUCC |
| | Cas9 | CGAAGACAGGAGACUCGCUGGCAAUGGCAAGAAGACUGGCAAGAUCGGUCAGAAGACUGACAAGAAGAAGAGCACAC |
| | coding | AGACUGCUGAGAACAAGAAGACUGCUGAAGAGAGAAGGAGUCCUGCAGGCAGCAAACUUCGACGAAAACGGACUGAU |
| | sequence | CAAGUCGCUGCCGAACACACCGUGGCAGCUGAGAGCAGCAGCACUGGACAGAAAGCUGACACCGCUGGAAUGGUCGGC |
| | | AGUCCUGCUGCACCUGAUCAAGCACAGAGGAUACCUGUCGCAGAGAAAGAACGAAGGAGAAACAGCAGACAAGGAAC |
| | | UGGGAGCACUGCUGAAGGGAGUCGCAGGAAACGCACACGCACUGCAGACAGGAGACUUCAGAACACCGGCAGAACUG |
| | | GCACUGAACAAGUUCGAAAAGGAAUCGGGACACAUCAGAAACCAGAGAUCGGACUACUCGCACACAUUCUCGAGAAA |
| | | GGACCUGCAGGCAGAACUGAUCCUGCUGUUCGAAAAGCAGAAGGAAUUCGGAAACCCGCACGUCUCGGGAGGACUGA |
| | | AGGAAGGAAUCGAAACACUGCUGAUGACACAGAGACCGGCACUGUCGGGAGACGCAGUCCAGAAGAUGCUGGGACAC |
| | | UGCACAUUCGAACCGGCAGAACCGAAGGCAGCAAAGAACACAUACACAGCAGAAAGAUUCAUCUGGCUGACAAAGCU |
| | | GAACAACCUGAGAAUCCUGGAACAGGGAUCGGAAAGACCGCUGACAGACACAGAAAGAGCAACACUGAUGGACGAAC |
| | | CGUACAGAAAGUCGAAGCUGACAUACGCACAGGCAAGAAAGCUGCUGGGACUGGAAGACACAGCAUUCUUCAAGGGA |
| | | CUGAGAUACGGAAAGGACAACGCAGAAGCAUCGACACUGAUGGAAAUGAAGGCAUACCACGCAAUCUCGAGAGCACU |
| | | GGAAAAGGAAGGACUGAAGGACAAGAAGUCGCCGCUGAACCUGUCGCCGGAACUGCAGGACGAAAUCGGAACAGCAU |
| | | UCUCGCUGUUCAAGACAGACGAAGACAUCACAGGAAGACUGAAGGACAGAAUCCAGCCGGAAAUCCUGGAAGCACUG |
| | | CUGAAGCACAUCUCGUUCGACAAGUUCGUCCAGAUCUCGCUGAAGGCACUGAGAAGAAUCGUCCCGCUGAUGGAACA |
| | | GGGAAAGAGAUACGACGAAGCAUGCGCAGAAAUCUACGGAGACCACUACGGAAAGAAGAACACAGAAGAAAAGAUCU |
| | | ACCUGCCGCCGAUCCCGGCAGACGAAAUCAGAAACCCGGUCGUCCUGAGAGCACUGUCGCAGGCAAGAAAGGUCAUCA |
| | | ACGGAGUCGUCAGAAGAUACGGAUCGCCGGCAAGAAUCCACAUCGAAACAGCAAGAGAAGUCGGAAAGUCGUUCAAG |
| | | GACAGAAAGGAAAUCGAAAAGAGACAGGAAGAAAACAGAAAGGACAGAGAAAAGGCAGCAGCAAAGUUCAGAGAAUA |
| | | CUUCCCGAACUUCGUCGGAGAACCGAAGUCGAAGGACAUCCUGAAGCUGAGACUGUACGAACAGCAGCACGGAAAGU |
| | | GCCUGUACUCGGGAAAGGAAAUCAACCUGGGAAGACUGAACGAAAAGGGAUACGUCGAAAUCGACCACGCACUGCCG |
| | | UUCUCGAGAACAUGGGACGACUCGUUCAACAACAAGGUCCUGGUCCUGGGAUCGGAAAACCAGAACAAGGGAAACCA |
| | | GACACCGUACGAAUACUUCAACGGAAAGGACAACUCGAGAGAAUGGCAGGAAUUCAAGGCAAGAGUCGAAACAUCGA |
| | | GAUUCCCGAGAUCGAAGAAGCAGAGAAUCCUGCUGCAGAAGUUCGACGAAGACGGAUUCAAGGAAAGAAACCUGAAC |
| | | GACACAAGAUACGUCAACAGAUUCCUGUGCCAGUUCGUCGCAGACAGAAUGAGACUGACAGGAAAGGGAAAGAAGAG |
| | | AGUCUUCGCAUCGAACGGACAGAUCACAAACCUGCUGAGAGGAUUCUGGGGACUGAGAAAGGUCAGAGCAGAAAACG |
| | | ACAGACACCACGCACUGGACGCAGUCGUCGUCGCAUGCUCGACAGUCGCAAUGCAGCAGAAGAUCACAAGAUUCGUCA |
| | | GAUACAAGGAAAUGAACGCAUUCGACGGAAAGACAAUCGACAAGGAAACAGGAGAAGUCCUGCACCAGAAGACACAC |
| | | UUCCCGCAGCCGUGGGAAUUCUUCGCACAGGAAGUCAUGAUCAGAGUCUUCGGAAAGCCGGACGGAAAGCCGGAAUU |
| | | CGAAGAAGCAGACACACUGGAAAAGCUGAGAACACUGCUGGCAGAAAAGCUGUCGUCGAGACCGGAAGCAGUCCACG |
| | | AAUACGUCACACCGCUGUUCGUCUCGAGAGCACCGAACAGAAAGAUGUCGGGACAGGGACACAUGGAAACAGUCAAG |
| | | UCGGCAAAGAGACUGGACGAAGGAGUCUCGGUCCUGAGAGUCCCGCUGACACAGCUGAAGCUGAAGGACCUGGAAAA |
| | | GAUGGUCAACAGAGAAAGAGAACCGAAGCUGUACGAAGCACUGAAGGCAAGACUGGAAGCACACAAGGACGACCCGG |
| | | CAAAGGCAUUCGCAGAACCGUUCUACAAGUACGACAAGGCAGGAAACAGAACACAGCAGGUCAAGGCAGUCAGAGUC |
| | | GAACAGGUCCAGAAGACAGGAGUCUGGGUCAGAAACCACAACGGAAUCGCAGACAACGCAACAAUGGUCAGAGUAGA |
| | | CGUCUUCGAAAAGGGAGACAAGUACUACCUGGUCCCGAUCUACUCGUGGCAGGUCGCAAAGGGAAUCCUGCCGGACA |
| | | GAGCAGUCGUCCAGGGAAAGGACGAAGAAGACUGGCAGCUGAUCGACGACUCGUUCAACUUCAAGUUCUCGCUGCAC |
| | | CCGAACGACCUGGUCGAAGUCAUCACAAAGAAGGCAAGAAUGUUCGGAUACUUCGCAUCGUGCCACAGAGGAACAGG |
| | | AAACAUCAACAUCAGAAUCCACGACCUGGACCACAAGAUCGGAAAGAACGGAAUCCUGGAAGGAAUCGGAGUCAAGA |
| | | CAGCACUGUCGUUCCAGAAGUACCAGAUCGACGAACUGGGAAAGGAAAUCAGACCGUGCAGACUGAAGAAGAGACCG |
| | | CCGGUCAGAUCCGGAAAGAGAACAGCAGACGGAUCGGAAUUCGAAUCGCCGAAGAAGAAGAGAAAGGUCGAAUGA |
| 107 | WT | MAAFKPNSINYILGLDIGIASVGWAMVEIDEEENPIRLIDLGVRVFERAEVPKTGDSLAMARRLARSVRRLTRRRAHRLLRTRRL |
| | Nme1 | LKREGVLQAANFDENGLIKSLPNTPWQLRAAALDRKLTPLEWSAVLLHLIKHRGYLSQRKNEGETADKELGALLKGVAGNAH |
| | Cas9 | ALQTGDFRTPAELALNKFEKESGHIRNQRSDYSHTFSRKDLQAELILLFEKQKEFGNPHVSGGLKEGIETLLMTQRPALSGDAVQ |
| | amino | KMLGHCTFEPAEPKAAKNTYTAERFIWLTKLNNLRILEQGSERPLTDTERATLMDEPYRKSKLTYAQARKLLGLEDTAFFKGLR |
| | acid | YGKDNAEASTLMEMKAYHAISRALEKEGLKDKKSPLNLSPELQDEIGTAFSLFKTDEDITGRLKDRIQPEILEALLKHISFDKFVQ |
| | sequence | ISLKALRRIVPLMEQGKRYDEACAEIYGDHYGKKNTEEKIYLPPIPADEIRNPVVLRALSQARKVINGVVRRYGSPARIHIETARE |
| | | VGKSFKDRKEIEKRQEENRKDREKAAAKFREYFPNFVGEPKSKDILKLRLYEQQHGKCLYSGKEINLGRLNEKGYVEIDHALPF |
| | | SRTWDDSFNNKVLVLGSENQNKGNQTPYEYFNGKDNSREWQEFKARVETSRFPRSKKQRILLQKFDEDGFKERNLNDTRYVN |
| | | RFLCQFVADRMRLTGKGKKRVFASNGQITNLLRGFWGLRKVRAENDRHHALDAVVVACSTVAMQQKITRFVRYKEMNAFDG |
| | | KTIDKETGEVLHQKTHFPQPWEFFAQEVMIRVFGKPDGKPEFEEADTLEKLRTLLAEKLSSRPEAVHEYVTPLFVSRAPNRKMS |
| | | GQGHMETVKSAKRLDEGVSVLRVPLTQLKLKDLEKMVNREREPKLYEALKARLEAHKDDPAKAFAEPFYKYDKAGNRTQQV |
| | | KAVRVEQVQKTGVWVRNHNGIADNATMVRVDVFEKGDKYYLVPIYSWQVAKGILPDRAVVQGKDEEDWQLIDDSFNFKFSL |
| | | HPNDLVEVITKKARMFGYFASCHRGTGNINIRIHDLDHKIGKNGILEGIGVKTALSFQKYQIDELGKEIRPCRLKKRPPVRSGKRT |
| | | ADGSEFESPKKKRKVE* |
| 108 | Nme2 | auggccgccuucaagcccaaccccaucaacuacauccugggccuggacaucggcaucgccuccgugggcugggccaugguggagaucgacg |
| | Cas9 | aggaggagaaccccauccggcugaucgaccugggcgugcggguguucgagcgggccgaggugcccaagaccggcgacucccuggccauggc |
| | coding | ccggcggcuggcccgguccgugcggcggcugacccggcggcgggcccaccggcugcugcgggcccggcggcugcugaagcgggagggcgug |
| | sequence | cugcaggccgccgacuucgacgagaacggccugaucaagucccugcccaacacccccuggcagcugcgggccgccgcccuggaccggaagc |
| | | ugaccccccuggagugguccgccgugcugcugcaccugaucaagcaccggggcuaccugucccagcggaagaacgagggcgagaccgccga |
| | | caaggagcugggcgcccugcugaagggcguggccaacaacgcccacgcccugcagaccggcgacuuccggacccccgccgagcuggcccug |
| | | aacaaguucgagaaggaguccggccacauccggaaccagcgggCggacuacucccacaccuucucccggaaggaccugcaggccgagcuga |
| | | uccugcuguucgagaagcagaaggaguucggcaacccccacguguccggggccugaaggagcggcaucgagacccugcugaugacccagcg |
| | | gcccgcccuguccggcgacgccgugcagaagaugcugggccacugcaccuucgagcccgccgagcccaaggccgccaagaacaccuacacc |
| | | gccgagcgguucaucuggcugaccaagcugaacaaccugcggauccuggagcagggcuccgagcggccccugaccgacaccgagggcgcca |
| | | cccugauggacgagcccuaccggaaguccaagcugaccuacgcccaggcccggaagcugcugggccuggaggacaccgccuucuucaaggg |
| | | ccugcgguacggcaaggacaacgccgaggccuccacccugauggagaugaaggccuaccacgccaucucccgggcccuggagaaggagggc |
| | | cugaaggacaagaaguccccccugaaccuguccuccgagcugcaggacgagaucggcaccgccuucucccuguucaagaccgacgaggaca |
| | | ucaccggccggcugaaggaccgggugcagcccgagauccuggaggcccugcugaagcacaucuccuucgacaaguucgugcagaucucccu |
| | | gaaggcccugcggcggaucgugccccugauggagcagggcaagcgguacgacgaggccugcgccgagaucuacggcgaccacuacggcaag |
| | | aagaacaccgaggagaagaucuaccugccccccauccccgccgacgagauccggaaccccguggugcugcgggcccugucccaggcccgga |
| | | aggugaucaacggcguggugcggcgguacggcucccccgcccggauccacaucgagaccgcccgggaggugggcaaguccuucaaggaccg |
| | | gaaggagaucgagaagcggcaggaggagaaccggaaggaccgggagaaggccgccgccaaguuccgggaguacuuccccaacuucgugggc |
| | | gagcccaaguccaaggacauccugaagcugcggcuguacgagcagcagcacggcaagugccuguacuccggcaaggagaucaaccuggugc |
| | | ggcugaacgagaagggcuacguggagaucgaccacgcccugcccuucucccggaccugggacgacuccuucaacaacaaggugcuggugcu |
| | | gggcuccgagaaccagaacaagggcaaccagacccccuacgaguacuucaacggcaaggacaacucccgggaguggcaggaguucaaggcc |
| | | cggguggagaccucccgguucccccgguccaagaagcagcggauccugcugcagaaguucgacgaggacggcuucaaggagugcaaccuga |
| | | acgacacccgguacgugaaccgguuccugugccaguucguggccgaccacauccugcugaccggcaaggcgcaaggggcgguguucgccuc |
| | | caacggccagaucaccaaccugcugcggggcuucuggggccugcggaagguggcggccgagaacgaccggcaccacgcccuggacgccgug |
| | | gugguggccugcuccaccguggccaugcagcagaagaucacccgguucgugcgguacaaggagaugaacgccuucgacggcaagaccaucg |
| | | acaaggagaccggcaaggugcugcaccagaagacccacuucccccagcccugggaguuuucgcccaggaggugaugauccgcgguguucgg |
| | | caagcccgacggcaagcccgaguucgaggaggccgacacccccgagaagcugcggacccugcuggccgagaagcuguccucccggcccgag |
| | | gccgugcacgaguacgugaccccccuguucgugucccgggcccccaaccggaagauguccggcgcccacaaggacacccugcgguccgcca |
| | | agcgguucgugaagcacaacgagaagaucuccgugaagcggguguggcugaccgagaucaagcuggccgaccuggagaacauggugaacua |
| | | caagaacggccgggagaucgagcuguacgaggcccugaaggcccggcuggaggccuacggcggcaacgccaagcaggccuucgaccccaag |
| | | gacaaccccuucuacaagaagggcggccagcuggugaaggccgugcggguggagaagacccaggaguccggcgugcugcugaacaagaaga |
| | | acgccuacaccaucgccgacaacggcgacauggugcggguggacguguucugcaagguggacaagaagggcaagaaccaguacuucaucgu |
| | | gcccaucuacgccuggcagguggccgagaacauccugcccgacaucgacugcaagggcuaccggaucgacgacuccuacaccuucugcuuc |
| | | ucccugcacaaguacgaccugaucgccuuccagaaggacgagaaguccaagguggaguucgccuacuacaucaacugcgacuccuccaacg |
| | | gccgguucuaccuggccuggcacgacaagggcuccaaggagcagcaguuccggaucuccacccagaaccuggugcugauccagaaguacca |
| | | ggugaacgagcugggcaaggagauccggcccugccggcugaagaagcggccccccgugcgg |
| 109 | Nme2 | MAAFKPNPINYILGLDIGIASVGWAMVEIDEEENPIRLIDLGVRVFERAEVPKTGDSLAMARRLARSVRRLTRRRAHRLLRARRLLKREGV |
| | Cas9 | LQAADFDENGLIKSLPNTPWQLRAAALDRKLTPLEWSAVLLHLIKHRGYLSQRKNEGETADKELGALLKGVANNAHALQTGDFRTPAELAL |
| | amino | NKFEKESGHIRNQRGDYSHTFSRKDLQAELILLFEKQKEFGNPHVSGGLKEGIETLLMTQRPALSGDAVQKMLGHCTFEPAEPKAAKNTYT |
| | acid | AERFIWLTKLNNLRILEQGSERPLTDTERATLMDEPYRKSKLTYAQARKLLGLEDTAFFKGLRYGKDNAEASTLMEMKAYHAISRALEKEG |
| | sequence | LKDKKSPLNLSSELQDEIGTAFSLFKTDEDITGRLKDRVQPEILEALLKHISFDKFVQISLKALRRIVPLMEQGKRYDEACAEIYGDHYGK |
| | | KNTEEKIYLPPIPADEIRNPVVLRALSQARKVINGVVRRYGSPARIHIETAREVGKSFKDRKEIEKRQEENRKDREKAAAKFREYFPNFVG |
| | | EPKSKDILKLRLYEQQHGKCLYSGKEINLVRLNEKGYVEIDHALPFSRTWDDSFNNKVLVLGSENQNKGNQTPYEYFNGKDNSREWQEFKA |
| | | RVETSRFPRSKKQRILLQKFDEDGFKECNLNDTRYVNRFLCQFVADHILLTGKGKRRVFASNGQITNLLRGFWGLRKVRAENDRHHALDAV |
| | | VVACSTVAMQQKITRFVRYKEMNAFDGKTIDKETGKVLHQKTHFPQPWEFFAQEVMIRVFGKPDGKPEFEEADTPEKLRTLLAEKLSSRPE |
| | | AVHEYVTPLFVSRAPNRKMSGAHKDTLRSAKRFVKHNEKISVKRVWLTEIKLADLENMVNYKNGREIELYEALKARLEAYGGNAKQAFDPK |
| | | DNPFYKKGGQLVKAVRVEKTQESGVLLNKKNAYTIADNGDMVRVDVFCKVDKKGKNQYFIVPIYAWQVAENILPDIDCKGYRIDDSYTFCF |
| | | SLHKYDLIAFQKDEKSKVEFAYYINCDSSNGRFYLAWHDKGSKEQQFRISTQNLVLIQKYQVNELGKEIRPCRLKKRPPVR |
| 110 | WT | AUGGACAAGAAGUACAGCAUCGGACUGGACAUCGGAACAAACAGCGUCGGAUGGGCAGUCAUCACAGACGAAUACAAGGUCCCGAGCAAGA |
| | SpyCas9- | AGUUCAAGGUCCUGGGAAACACAGACAGACACAGCAUCAAGAAGAACCUGAUCGGAGCACUGCUGUUCGACAGCGGAGAAACAGCAGAAGC |
| | ORF | AACAAGACUGAAGAGAACAGCAAGAAGAAGAUACACAAGAAGAAAGAACAGAAUCUGCUACCUGCAGGAAAUCUUCAGCAACGAAAUGGCA |
| | sequence | AAGGUCGACGACAGCUUCUUCCACAGACUGGAAGAAAGCUUCCUGGUCGAAGAAGACAAGAAGCACGAAAGACACCCGAUCUUCGGAAACA |
| | | UCGUCGACGAAGUCGCAUACCACGAAAAGUACCCGACAAUCUACCACCUGAGAAAGAAGCUGGUCGACAGCACAGACAAGGCAGACCUGAG |
| | | ACUGAUCUACCUGGCACUGGCACACAUGAUCAAGUUCAGAGGACACUUCCUGAUCGAAGGAGACCUGAACCCGGACAACAGCGACGUCGAC |
| | | AAGCUGUUCAUCCAGCUGGUCCAGACAUACAACCAGCUGUUCGAAGAAAACCCGAUCAACGCAAGCGGAGUCGACGCAAAGGCAAUCCUGA |
| | | GCGCAAGACUGAGCAAGAGCAGAAGACUGGAAAACCUGAUCGCACAGCUGCCGGGAGAAAAGAAGAACGGACUGUUCGGAAACCUGAUCGC |
| | | ACUGAGCCUGGGACUGACACCGAACUUCAAGAGCAACUUCGACCUGGCAGAAGACGCAAAGCUGCAGCUGAGCAAGGACACAUACGACGAC |
| | | GACCUGGACAACCUGCUGGCACAGAUCGGAGACCAGUACGCAGACCUGUUCCUGGCAGCAAAGAACCUGAGCGACGCAAUCCUGCUGAGCG |
| | | ACAUCCUGAGAGUCAACACAGAAAUCACAAAGGCACCGCUGAGCGCAAGCAUGAUCAAGAGAUACGACGAACACCACCAGGACCUGACACU |
| | | GCUGAAGGCACUGGUCAGACAGCAGCUGCCGGAAAAGUACAAGGAAAUCUUCUUCGACCAGAGCAAGAACGGAUACGCAGGAUACAUCGAC |
| | | GGAGGAGCAAGCCAGGAAGAAUUCUACAAGUUCAUCAAGCCGAUCCUGGAAAAGAUGGACGGAACAGAAGAACUGCUGGUCAAGCUGAACA |
| | | GAGAAGACCUGCUGAGAAAGCAGAGAACAUUCGACAACGGAAGCAUCCCGCACCAGAUCCACCUGGGAGAACUGCACGCAAUCCUGAGAAG |
| | | ACAGGAAGACUUCUACCCGUUCCUGAAGGACAACAGAGAAAAGAUCGAAAAGAUCCUGACAUUCAGAAUCCCGUACUACGUCGGACCGCUG |
| | | GCAAGAGGAAACAGCAGAUUCGCAUGGAUGACAAGAAAGAGCGAAGAAACAAUCACACCGUGGAACUUCGAAGAAGUCGUCGACAAGGGAG |
| | | CAAGCGCACAGAGCUUCAUCGAAAGAAUGACAAACUUCGACAAGAACCUGCCGAACGAAAAGGUCCUGCCGAAGCACAGCCUGCUGUACGA |
| | | AUACUUCACAGUCUACAACGAACUGACAAAGGUCAAGUACGUCACAGAAGGAAUGAGAAAGCCGGCAUUCCUGAGCGGAGAACAGAAGAAG |
| | | GCAAUCGUCGACCUGCUGUUCAAGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGAUCAUCAAGGACAAGGACUUCCU |
| | | GGACAACGAAGAAAACGAAGACAUCCUGGAAGACAUCGUCCUGACACUGACACUGUUCGAAGACAGAGAAAUGAUCGAAGAAAGACUGAAG |
| | | ACAUACGCACACCUGUUCGACGACAAGGUCAUGAAGCAGCUGAAGAGAAGAAGAUACACAGGAUGGGGAAGACUGAGCAGAAAGCUGAUCA |
| | | ACGGAAUCAGAGACAAGCAGAGCGGAAAGACAAUCCUGGACUUCCUGAAGAGCGACGGAUUCGCAAACAGAAACUUCAUGCAGCUGAUCCA |
| | | CGACGACAGCCUGACAUUCAAGGAAGACAUCCAGAAGGCACAGGUCAGCGGACAGGGAGACAGCCUGCACGAACACAUCGCAAACCUGGCA |
| | | GGAAGCCCGGCAAUCAAGAAGGGAAUCCUGCAGACAGUCAAGGUCGUCGACGAACUGGUCAAGGUCAUGGGAAGACACAAGCCGGAAAACA |
| | | UCGUCAUCGAAAUGGCAAGAGAAAACCAGACAACACAGAAGGGACAGAAGAACAGCAGAGAAAGAAUGAAGAGAAUCGAAGAAGGAAUCAA |
| | | GGAACUGGGAAGCCAGAUCCUGAAGGAACACCCGGUCGAAAACACACAGCUGCAGAACGAAAAGCUGUACCUGUACUACCUGCAGAACGGA |
| | | AGAGACAUGUACGUCGACCAGGAACUGGACAUCAACAGACUGAGCGACUACGACGUCGACCACAUCGUCCCGCAGAGCUUCCUGAAGGACG |
| | | ACAGCAUCGACAACAAGGUCCUGACAAGAAGCGACAAGAACAGAGGAAAGAGCGACAACGUCCCGAGCGAAGAAGUCGUCAAGAAGAUGAA |
| | | GAACUACUGGAGACAGCUGCUGAACGCAAAGCUGAUCACACAGAGAAAGUUCGACAACCUGACAAAGGCAGAGAGAGGAGGACUGAGCGAA |
| | | CUGGACAAGGCAGGAUUCAUCAAGAGACAGCUGGUCGAAACAAGACAGAUCACAAAGCACGUCGCACAGAUCCUGGACAGCAGAAUGAACA |
| | | CAAAGUACGACGAAAACGACAAGCUGAUCAGAGAAGUCAAGGUCAUCACACUGAAGAGCAAGCUGGUCAGCGACUUCAGAAAGGACUUCCA |
| | | GUUCUACAAGGUCAGAGAAAUCAACAACUACCACCACGCACACGACGCAUACCUGAACGCAGUCGUCGGAACAGCACUGAUCAAGAAGUAC |
| | | CCGAAGCUGGAAAGCGAAUUCGUCUACGGAGACUACAAGGUCUACGACGUCAGAAAGAUGAUCGCAAAGAGCGAACAGGAAAUCGGAAAGG |
| | | CAACAGCAAAGUACUUCUUCUACAGCAACAUCAUGAACUUCUUCAAGACAGAAAUCACACUGGCAAACGGAGAAAUCAGAAAGAGACCGCU |
| | | GAUCGAAACAAACGGAGAAACAGGAGAAAUCGUCUGGGACAAGGGAAGAGACUUCGCAACAGUCAGAAAGGUCCUGAGCAUGCCGCAGGUC |
| | | AACAUCGUCAAGAAGACAGAAGUCCAGACAGGAGGAUUCAGCAAGGAAAGCAUCCUGCCGAAGAGAAACAGCGACAAGCUGAUCGCAAGAA |
| | | AGAAGGACUGGGACCCGAAGAAGUACGGAGGAUUCGACAGCCCGACAGUCGCAUACAGCGUCCUGGUCGUCGCAAAGGUCGAAAAGGGAAA |
| | | GAGCAAGAAGCUGAAGAGCGUCAAGGAACUGCUGGGAAUCACAAUCAUGGAAAGAAGCAGCUUCGAAAAGAACCCGAUCGACUUCCUGGAA |
| | | GCAAAGGGAUACAAGGAAGUCAAGAAGGACCUGAUCAUCAAGCUGCCGAAGUACAGCCUGUUCGAACUGGAAAACGGAAGAAAGAGAAUGC |
| | | UGGCAAGCGCAGGAGAACUGCAGAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGACGAAAUCAUCGAACAGAUCAGC |
| | | GAAUUCAGCAAGAGAGUCAUCCUGGCAGACGCAAACCUGGACAAGGUCCUGAGCGCAUACAACAAGCACAGAGACAAGCCGAUCAGAGAAC |
| | | AGGCAGAAAACAUCAUCCACCUGUUCACACUGACAAACCUGGGAGCACCGGCAGCAUUCAAGUACUUCGACACAACAAUCGACAGAAAGAG |
| | | AUACACAAGCACAAAGGAAGUCCUGGACGCAACACUGAUCCACCAGAGCAUCACAGGACUGUACGAAACAAGAAUCGACCUGAGCCAGCUG |
| | | GGAGGAGACGGAGGAGGAAGCCCGAAGAAGAAGAGAAAGGUCUAG |
| 111 | WT | MDKKYSIGLDIGTNSVGWAVITDEYKVPSKKFKVLGNTDRHSIKKNLIGALLFDSGETAEATRLKRTARRRYTRRKNRICYLQEIFSNEMA |
| | SpyCas9- | KVDDSFFHRLEESFLVEEDKKHERHPIFGNIVDEVAYHEKYPTIYHLRKKLVDSTDKADLRLIYLALAHMIKFRGHFLIEGDLNPDNSDVD |
| | amino | KLFIQLVQTYNQLFEENPINASGVDAKAILSARLSKSRRLENLIAQLPGEKKNGLFGNLIALSLGLTPNFKSNFDLAEDAKLQLSKDTYDD |
| | acid | DLDNLLAQIGDQYADLFLAAKNLSDAILLSDILRVNTEITKAPLSASMIKRYDEHHQDLTLLKALVRQQLPEKYKEIFFDQSKNGYAGYID |
| | sequence | GGASQEEFYKFIKPILEKMDGTEELLVKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGTYHDLLKIIKDKDFLDNEENEDILEDIVLTLTLFEDREMIEERLK |
| | | TYAHLFDDKVMKQLKRRRYTGWGRLSRKLINGIRDKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQFYKVREINNYHHAHDAYLNAVVGTALIKKY |
| | | PKLESEFVYGDYKVYDVRKMIAKSEQEIGKATAKYFFYSNIMNFFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTVAYSVLVVAKVEKGKSKKLKSVKELLGITIMERSSFEKNPIDFLE |
| | | AKGYKEVKKDLIIKLPKYSLFELENGRKRMLASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKRYTSTKEVLDATLIHQSITGLYETRIDLSQL |
| | | GGDGGGSPKKKRKV* |
| 112- | | NQT USED |
| 119 | | |
| 120 | Exemplary | MAAFKPNSINYILGLDIGIASVGWAMVEIDEEENPIRLIDLGVRVFERAEVPKTGDSLAMARRLARSVRRLTRRRAHRLLRTRRLLKREGV |
| | amino | LQAANFDENGLIKSLPNTPWQLRAAALDRKLTPLEWSAVLLHLIKHRGYLSQRKNEGETADKELGALLKGVAGNAHALQTGDFRTPAELAL |
| | acid | NKFEKESGHIRNQRSDYSHTFSRKDLQAELILLFEKQKEFGNPHVSGGLKEGIETLLMTQRPALSGDAVQKMLGHCTFEPAEPKAAKNTYT |
| | sequence | AERFIWLTKLNNLRILEQGSERPLTDTERATLMDEPYRKSKLTYAQARKLLGLEDTAFFKGLRYGKDNAEASTLMEMKAYHAISRALEKEG |
| | of | LKDKKSPLNLSPELQDEIGTAFSLFKTDEDITGRLKDRIQPEILEALLKHISFDKFVQISLKALRRIVPLMEQGKRYDEACAEIYGDHYGK |
| | Nme1Cas9 | KNTEEKIYLPPIPADEIRNPVVLRALSQARKVINGVVRRYGSPARIHIETAREVGKSFKDRKEIEKRQEENRKDREKAAAKFREYFPNFVG |
| | cleavase | EPKSKDILKLRLYEQQHGKCLYSGKEINLGRLNEKGYVEIDHALPFSRTWDDSFNNKVLVLGSENQNKGNQTPYEYFNGKDNSREWQEFKA |
| | | RVETSRFPRSKKQRILLQKFDEDGFKERNLNDTRYVNRFLCQFVADRMRLTGKGKKRVFASNGQITNLLRGFWGLRKVRAENDRHHALDAV |
| | | VVACSTVAMQQKITRFVRYKEMNAFDGKTIDKETGEVLHQKTHFPQPWEFFAQEVMIRVFGKPDGKPEFEEADTLEKLRTLLAEKLSSRPE |
| | | AVHEYVTPLFVSRAPNRKMSGQGHMETVKSAKRLDEGVSVLRVPLTQLKLKDLEKMVNREREPKLYEALKARLEAHKDDPAKAFAEPFYKY |
| | | DKAGNRTQQVKAVRVEQVQKTGVWVRNHNGIADNATMVRVDVFEKGDKYYLVPIYSWQVAKGILPDRAVVQGKDEEDWQLIDDSFNFKFSL |
| | | HPNDLVEVITKKARMFGYFASCHRGTGNINIRIHDLDHKIGKNGILEGIGVKTALSFQKYQIDELGKEIRPCRLKKRPPVR |
| 121 | Exemplary | GCTGCTTTTAAGCCTAATTCTATTAATTATATTCTTGGTCTTGATATTGGTATTGCTTCTGTTGGTTGGGCTATGGTTGAGATTGATGAGG |
| | coding | AGGAGAATCCTATTCGTCTTATTGATCTTGGTGTTCGTGTTTTTGAGCGTGCTGAGGTTCCTAAGACTGGTGATTCTCTTGCTATGGCTCG |
| | sequence | TCGTCTTGCTCGTTCTGTTCGTCGTCTTACTCGTCGTCGTGCTCATCGTCTTCTTCGTACTCGTCGTCTTCTTAAGCGTGAGGGTGTTCTT |
| | encoding | CAGGCTGCTAATTTTGATGAGAATGGTCTTATTAAGTCTCTTCCTAATACTCCTTGGCAGCTTCGTGCTGCTGCTCTTGATCGTAAGCTTA |
| | Nme1Cas9 | CTCCTCTTGAGTGGTCTGCTGTTCTTCTTCATCTTATTAAGCATCGTGGTTATCTTTCTCAGCGTAAGAATGAGGGTGAGACTGCTGATAA |
| | cleavase | GGAGCTTGGTGCTCTTCTTAAGGGTGTTGCTGGTAATGCTCATGCTCTTCAGACTGGTGATTTTCGTACTCCTGCTGAGCTTGCTCTTAAT |
| | | AAGTTTGAGAAGGAGTCTGGTCATATTCGTAATCAGCGTTCTGATTATTCTCATACTTTTTCTCGTAAGGATCTTCAGGCTGAGCTTATTC |
| | | TTCTTTTTGAGAAGCAGAAGGAGTTTGGTAATCCTCATGTTTCTGGTGGTCTTAAGGAGGGTATTGAGACTCTTCTTATGACTCAGCGTCC |
| | | TGCTCTTTCTGGTGATGCTGTTCAGAAGATGCTTGGTCATTGTACTTTTGAGCCTGCTGAGCCTAAGGCTGCTAAGAATACTTATACTGCT |
| | | GAGCGTTTTATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTGGTCTTGAGGATACTGCTTTTTTTAAGGGTCT |
| | | TCGTTATGGTAAGGATAATGCTGAGGCTTCTACTCTTATGGAGATGAAGGCTTATCATGCTATTTCTCGTGCTCTTGAGAAGGAGGGTCTT |
| | | AAGGATAAGAAGTCTCCTCTTAATCTTTCTCCTGAGCTTCAGGATGAGATTGGTACTGCTTTTTCTCTTTTTAAGACTGATGAGGATATTA |
| | | CTGGTCGTCTTAAGGATCGTATTCAGCCTGAGATTCTTGAGGCTCTTCTTAAGCATATTTCTTTTGATAAGTTTGTTCAGATTTCTCTTAA |
| | | GGCTCTTCGTCGTATTGTTCCTCTTATGGAGCAGGGTAAGCGTTATGATGAGGCTTGTGCTGAGATTTATGGTGATCATTATGGTAAGAAG |
| | | AATACTGAGGAGAAGATTTATCTTCCTCCTATTCCTGCTGATGAGATTCGTAATCCTGTTGTTCTTCGTGCTCTTTCTCAGGCTCGTAAGG |
| | | TTATTAATGGTGTTGTTCGTCGTTATGGTTCTCCTGCTCGTATTCATATTGAGACTGCTCGTGAGGTTGGTAAGTCTTTTAAGGATCGTAA |
| | | GGAGATTGAGAAGCGTCAGGAGGAGAATCGTAAGGATCGTGAGAAGGCTGCTGCTAAGTTTCGTGAGTATTTTCCTAATTTTGTTGGTGAG |
| | | CCTAAGTCTAAGGATATTCTTAAGCTTCGTCTTTATGAGCAGCAGCATGGTAAGTGTCTTTATTCTGGTAAGGAGATTAATCTTGGTCGTC |
| | | TTAATGAGAAGGGTTATGTTGAGATTGATCATGCTCTTCCTTTTTCTCGTACTTGGGATGATTCTTTTAATAATAAGGTTCTTGTTCTTGG |
| | | TTCTGAGAATCAGAATAAGGGTAATCAGACTCCTTATGAGTATTTTAATGGTAAGGATAATTCTCGTGAGTGGCAGGAGTTTAAGGCTCGT |
| | | GTTGAGACTTCTCGTTTTCCTCGTTCTAAGAAGCAGCGTATTCTTCTTCAGAAGTTTGATGAGGATGGTTTTAAGGAGCGTAATCTTAATG |
| | | ATACTCGTTATGTTAATCGTTTTCTTTGTCAGTTTGTTGCTGATCGTATGCGTCTTACTGGTAAGGGTAAGAAGCGTGTTTTTGCTTCTAA |
| | | TGGTCAGATTACTAATCTTCTTCGTGGTTTTTGGGGTCTTCGTAAGGTTCGTGCTGAGAATGATCGTCATCATGCTCTTGATGCTGTTGTT |
| | | GTTGCTTGTTCTACTGTTGCTATGCAGCAGAAGATTACTCGTTTTGTTCGTTATAAGGAGATGAATGCTTTTGATGGTAAGACTATTGATA |
| | | AGGAGACTGGTGAGGTTCTTCATCAGAAGACTCATTTTCCTCAGCCTTGGGAGTTTTTTGCTCAGGAGGTTATGATTCGTGTTTTTGGTAA |
| | | GCCTGATGGTAAGCCTGAGTTTGAGGAGGCTGATACTCTTGAGAAGCTTCGTACTCTTCTTGCTGAGAAGCTTTCTTCTCGTCCTGAGGCT |
| | | GTTCATGAGTATGTTACTCCTCTTTTTGTTTCTCGTGCTCCTAATCGTAAGATGTCTGGTCAGGGTCATATGGAGACTGTTAAGTCTGCTA |
| | | AGCGTCTTGATGAGGGTGTTTCTGTTCTTCGTGTTCCTCTTACTCAGCTTAAGCTTAAGGATCTTGAGAAGATGGTTAATCGTGAGCGTGA |
| | | GCCTAAGCTTTATGAGGCTCTTAAGGCTCGTCTTGAGGCTCATAAGGATGATCCTGCTAAGGCTTTTGCTGAGCCTTTTTATAAGTATGAT |
| | | AAGGCTGGTAATCGTACTCAGCAGGTTAAGGCTGTTCGTGTTGAGCAGGTTCAGAAGACTGGTGTTTGGGTTCGTAATCATAATGGTATTG |
| | | CTGATAATGCTACTATGGTTCGTGTTGATGTTTTTGAGAAGGGTGATAAGTATTATCTTGTTCCTATTTATTCTTGGCAGGTTGCTAAGGG |
| | | TATTCTTCCTGATCGTGCTGTTGTTCAGGGTAAGGATGAGGAGGATTGGCAGCTTATTGATGATTCTTTTAATTTTAAGTTTTCTCTTCAT |
| | | CCTAATGATCTTGTTGAGGTTATTACTAAGAAGGCTCGTATGTTTGGTTATTTTGCTTCTTGTCATCGTGGTACTGGTAATATTAATATTC |
| | | GTATTCATGATCTTGATCATAAGATTGGTAAGAATGGTATTCTTGAGGGTATTGGTGTTAAGACTGCTCTTTCTTTTCAGAAGTATCAGAT |
| | | TGATGAGCTTGGTAAGGAGATTCGTCCTTGTCGTCTTAAGAAGCGTCCTCCTGTTCGT |
| 122 | Exemplary | GCCGCCTTCAAGCCCAACTCCATCAACTACATCCTGGGCCTGGACATCGGCATCGCCTCCGTGGGCTGGGCCATGGTGGAGATCGACGAGG |
| | coding | AGGAGAACCCCATCCGGCTGATCGACCTGGGCGTGCGGGTGTTCGAGCGGGCCGAGGTGCCCAAGACCGGCGACTCCCTGGCCATGGCCCG |
| | sequence | GCGGCTGGCCCGGTCCGTGCGGCGGCTGACCCGGCGGCGGGCCCACCGGCTGCTGCGGACCCGGCGGCTGCTGAAGCGGGAGGGCGTGCTG |
| | | CAGGCCGCCAACTTCGACGAGAACGGCCTGATCAAGTCCCTGCCCAACACCCCCTGGCAGCTGCGGGCCGCCGCCCTGGACCGGAAGCTGA |
| | encoding | CCCCCCTGGAGTGGTCCGCCGTGCTGCTGCACCTGATCAAGCACCGGGGCTACCTGTCCCAGCGGAAGAACGAGGGCGAGACCGCCGACAA |
| | Nme1Cas9 | GGAGCTGGGCGCCCTGCTGAAGGGCGTGGCCGGCAACGCCCACGCCCTGCAGACCGGCGACTTCCGGACCCCCGCCGAGCTGGCCCTGAAC |
| | cleavase | AAGTTCGAGAAGGAGTCCGGCCACATCCGGAACCAGCGGTCCGACTACTCCCACACCTTCTCCCGGAAGGACCTGCAGGCCGAGCTGATCC |
| | | TGCTGTTCGAGAAGCAGAAGGAGTTCGGCAACCCCCACGTGTCCGGCGGCCTGAAGGAGGGCATCGAGACCCTGCTGATGACCCAGCGGCC |
| | | CGCCCTGTCCGGCGACGCCGTGCAGAAGATGCTGGGCCACTGCACCTTCGAGCCCGCCGAGCCCAAGGCCGCCAAGAACACCTACACCGCC |
| | | GAGCGGTTCATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGGGCCTGGAGGACACCGCCTTCTTCAAGGGCCT |
| | | GCGGTACGGCAAGGACAACGCCGAGGCCTCCACCCTGATGGAGATGAAGGCCTACCACGCCATCTCCCGGGCCCTGGAGAAGGAGGGCCTG |
| | | AAGGACAAGAAGTCCCCCCTGAACCTGTCCCCCGAGCTGCAGGACGAGATCGGCACCGCCTTCTCCCTGTTCAAGACCGACGAGGACATCA |
| | | CCGGCCGGCTGAAGGACCGGATCCAGCCCGAGATCCTGGAGGCCCTGCTGAAGCACATCTCCTTCGACAAGTTCGTGCAGATCTCCCTGAA |
| | | GGCCCTGCGGCGGATCGTGCCCCTGATGGAGCAGGGCAAGCGGTACGACGAGGCCTGCGCCGAGATCTACGGCGACCACTACGGCAAGAAG |
| | | AACACCGAGGAGAAGATCTACCTGCCCCCCATCCCCGCCGACGAGATCCGGAACCCCGTGGTGCTGCGGGCCCTGTCCCAGGCCCGGAAGG |
| | | TGATCAACGGCGTGGTGCGGCGGTACGGCTCCCCCGCCCGGATCCACATCGAGACCGCCCGGGAGGTGGGCAAGTCCTTCAAGGACCGGAA |
| | | GGAGATCGAGAAGCGGCAGGAGGAGAACCGGAAGGACCGGGAGAAGGCCGCCGCCAAGTTCCGGGAGTACTTCCCCAACTTCGTGGGCGAG |
| | | CCCAAGTCCAAGGACATCCTGAAGCTGCGGCTGTACGAGCAGCAGCACGGCAAGTGCCTGTACTCCGGCAAGGAGATCAACCTGGGCCGGC |
| | | TGAACGAGAAGGGCTACGTGGAGATCGACCACGCCCTGCCCTTCTCCCGGACCTGGGACGACTCCTTCAACAACAAGGTGCTGGTGCTGGG |
| | | CTCCGAGAACCAGAACAAGGGCAACCAGACCCCCTACGAGTACTTCAACGGCAAGGACAACTCCCGGGAGTGGCAGGAGTTCAAGGCCCGG |
| | | GTGGAGACCTCCCGGTTCCCCCGGTCCAAGAAGCAGCGGATCCTGCTGCAGAAGTTCGACGAGGACGGCTTCAAGGAGCGGAACCTGAACG |
| | | ACACCCGGTACGTGAACCGGTTCCTGTGCCAGTTCGTGGCCGACCGGATGCGGCTGACCGGCAAGGGCAAGAAGCGGGTGTTCGCCTCCAA |
| | | CGGCCAGATCACCAACCTGCTGCGGGGCTTCTGGGGCCTGCGGAAGGTGCGGGCCGAGAACGACCGGCACCACGCCCTGGACGCCGTGGTG |
| | | GTGGCCTGCTCCACCGTGGCCATGCAGCAGAAGATCACCCGGTTCGTGCGGTACAAGGAGATGAACGCCTTCGACGGCAAGACCATCGACA |
| | | AGGAGACCGGCGAGGTGCTGCACCAGAAGACCCACTTCCCCCAGCCCTGGGAGTTCTTCGCCCAGGAGGTGATGATCCGGGTGTTCGGCAA |
| | | GCCCGACGGCAAGCCCGAGTTCGAGGAGGCCGACACCCTGGAGAAGCTGCGGACCCTGCTGGCCGAGAAGCTGTCCTCCCGGCCCGAGGCC |
| | | GTGCACGAGTACGTGACCCCCCTGTTCGTGTCCCGGGCCCCCAACCGGAAGATGTCCGGCCAGGGCCACATGGAGACCGTGAAGTCCGCCA |
| | | AGCGGCTGGACGAGGGCGTGTCCGTGCTGCGGGTGCCCCTGACCCAGCTGAAGCTGAAGGACCTGGAGAAGATGGTGAACCGGGAGCGGGA |
| | | GCCCAAGCTGTACGAGGCCCTGAAGGCCCGGCTGGAGGCCCACAAGGACGACCCCGCCAAGGCCTTCGCCGAGCCCTTCTACAAGTACGAC |
| | | AAGGCCGGCAACCGGACCCAGCAGGTGAAGGCCGTGCGGGTGGAGCAGGTGCAGAAGACCGGCGTGTGGGTGCGGAACCACAACGGCATCG |
| | | CCGACAACGCCACCATGGTGCGGGTGGACGTGTTCGAGAAGGGCGACAAGTACTACCTGGTGCCCATCTACTCCTGGCAGGTGGCCAAGGG |
| | | CATCCTGCCCGACCGGGCCGTGGTGCAGGGCAAGGACGAGGAGGACTGGCAGCTGATCGACGACTCCTTCAACTTCAAGTTCTCCCTGCAC |
| | | CCCAACGACCTGGTGGAGGTGATCACCAAGAAGGCCCGGATGTTCGGCTACTTCGCCTCCTGCCACCGGGGCACCGGCAACATCAACATCC |
| | | GGATCCACGACCTGGACCACAAGATCGGCAAGAACGGCATCCTGGAGGGCATCGGCGTGAAGACCGCCCTGTCCTTCCAGAAGTACCAGAT |
| | | CGACGAGCTGGGCAAGGAGATCCGGCCCTGCCGGCTGAAGAAGCGGCCCCCCGTGCGG |
| 123 | Exemplary | GCAGCATTCAAACCAAACTCAATCAACTACATCCTAGGACTAGACATCGGAATCGCATCAGTAGGATGAGCAATGGTAGAAATCGACGAAG |
| | coding | AAGAAAACCCAATCCGACTAATCGACCTAGGAGTACGAGTATTCGAACGAGCAGAAGTACCAAAAACAGGAGACTCACTAGCAATGGCACG |
| | sequence | ACGACTAGCACGATCAGTACGACGACTAACACGACGACGAGCACACCGACTACTACGAACACGACGACTACTAAAACGAGAAGGAGTACTA |
| | encoding | CAAGCAGCAAACTTCGACGAAAACGGACTAATCAAATCACTACCAAACACACCATGACAACTACGAGCAGCAGCACTAGACCGAAAACTAA |
| | Nme1Cas9 | CACCACTAGAATGATCAGCAGTACTACTACACCTAATCAAACACCGAGGATACCTATCACAACGAAAAAACGAAGGAGAAACAGCAGACAA |
| | cleavase | AGAACTAGGAGCACTACTAAAAGGAGTAGCAGGAAACGCACACGCACTACAAACAGGAGACTTCCGAACACCAGCAGAACTAGCACTAAAC |
| | | AAATTCGAAAAAGAATCAGGACACATCCGAAACCAACGATCAGACTACTCACACACATTCTCACGAAAAGACCTACAAGCAGAACTAATCC |
| | | TACTATTCGAAAAACAAAAAGAATTCGGAAACCCACACGTATCAGGAGGACTAAAAGAAGGAATCGAAACACTACTAATGACACAACGACC |
| | | AGCACTATCAGGAGACGCAGTACAAAAAATGCTAGGACACTGCACATTCGAACCAGCAGAACCAAAAGCAGCAAAAAACACATACACAGCA |
| | | GAACGATTCATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTAGGACTAGAAGACACAGCATTCTTCAAAGGACT |
| | | ACGATACGGAAAAGACAACGCAGAAGCATCAACACTAATGGAAATGAAAGCATACCACGCAATCTCACGAGCACTAGAAAAAGAAGGACTA |
| | | AAAGACAAAAAATCACCACTAAACCTATCACCAGAACTACAAGACGAAATCGGAACAGCATTCTCACTATTCAAAACAGACGAAGACATCA |
| | | CAGGACGACTAAAAGACCGAATCCAACCAGAAATCCTAGAAGCACTACTAAAACACATCTCATTCGACAAATTCGTACAAATCTCACTAAA |
| | | AGCACTACGACGAATCGTACCACTAATGGAACAAGGAAAACGATACGACGAAGCATGCGCAGAAATCTACGGAGACCACTACGGAAAAAAA |
| | | AACACAGAAGAAAAAATCTACCTACCACCAATCCCAGCAGACGAAATCCGAAACCCAGTAGTACTACGAGCACTATCACAAGCACGAAAAG |
| | | TAATCAACGGAGTAGTACGACGATACGGATCACCAGCACGAATCCACATCGAAACAGCACGAGAAGTAGGAAAATCATTCAAAGACCGAAA |
| | | AGAAATCGAAAAACGACAAGAAGAAAACCGAAAAGACCGAGAAAAAGCAGCAGCAAAATTCCGAGAATACTTCCCAAACTTCGTAGGAGAA |
| | | CCAAAATCAAAAGACATCCTAAAACTACGACTATACGAACAACAACACGGAAAATGCCTATACTCAGGAAAAGAAATCAACCTAGGACGAC |
| | | TAAACGAAAAAGGATACGTAGAAATCGACCACGCACTACCATTCTCACGAACATGAGACGACTCATTCAACAACAAAGTACTAGTACTAGG |
| | | ATCAGAAAACCAAAACAAAGGAAACCAAACACCATACGAATACTTCAACGGAAAAGACAACTCACGAGAATGACAAGAATTCAAAGCACGA |
| | | GTAGAAACATCACGATTCCCACGATCAAAAAAACAACGAATCCTACTACAAAAATTCGACGAAGACGGATTCAAAGAACGAAACCTAAACG |
| | | ACACACGATACGTAAACCGATTCCTATGCCAATTCGTAGCAGACCGAATGCGACTAACAGGAAAAGGAAAAAAACGAGTATTCGCATCAAA |
| | | CGGACAAATCACAAACCTACTACGAGGATTCTGAGGACTACGAAAAGTACGAGCAGAAAACGACCGACACCACGCACTAGACGCAGTAGTA |
| | | GTAGCATGCTCAACAGTAGCAATGCAACAAAAAATCACACGATTCGTACGATACAAAGAAATGAACGCATTCGACGGAAAAACAATCGACA |
| | | AAGAAACAGGAGAAGTACTACACCAAAAAACACACTTCCCACAACCATGAGAATTCTTCGCACAAGAAGTAATGATCCGAGTATTCGGAAA |
| | | ACCAGACGGAAAACCAGAATTCGAAGAAGCAGACACACTAGAAAAACTACGAACACTACTAGCAGAAAAACTATCATCACGACCAGAAGCA |
| | | GTACACGAATACGTAACACCACTATTCGTATCACGAGCACCAAACCGAAAAATGTCAGGACAAGGACACATGGAAACAGTAAAATCAGCAA |
| | | AACGACTAGACGAAGGAGTATCAGTACTACGAGTACCACTAACACAACTAAAACTAAAAGACCTAGAAAAAATGGTAAACCGAGAACGAGA |
| | | ACCAAAACTATACGAAGCACTAAAAGCACGACTAGAAGCACACAAAGACGACCCAGCAAAAGCATTCGCAGAACCATTCTACAAATACGAC |
| | | AAAGCAGGAAACCGAACACAACAAGTAAAAGCAGTACGAGTAGAACAAGTACAAAAAACAGGAGTATGAGTACGAAACCACAACGGAATCG |
| | | CAGACAACGCAACAATGGTACGAGTAGACGTATTCGAAAAAGGAGACAAATACTACCTAGTACCAATCTACTCATGACAAGTAGCAAAAGG |
| | | AATCCTACCAGACCGAGCAGTAGTACAAGGAAAAGACGAAGAAGACTGACAACTAATCGACGACTCATTCAACTTCAAATTCTCACTACAC |
| | | CCAAACGACCTAGTAGAAGTAATCACAAAAAAAGCACGAATGTTCGGATACTTCGCATCATGCCACCGAGGAACAGGAAACATCAACATCC |
| | | GAATCCACGACCTAGACCACAAAATCGGAAAAAACGGAATCCTAGAAGGAATCGGAGTAAAAACAGCACTATCATTCCAAAAATACCAAAT |
| | | CGACGAACTAGGAAAAGAAATCCGACCATGCCGACTAAAAAAACGACCACCAGTACGA |
| 124 | Exemplary | ATGGCTGCTTTTAAGCCTAATTCTATTAATTATATTCTTGGTCTTGATATTGGTATTGCTTCTGTTGGTTGGGCTATGGTTGAGATTGATG |
| | open | AGGAGGAGAATCCTATTCGTCTTATTGATCTTGGTGTTCGTGTTTTTGAGCGTGCTGAGGTTCCTAAGACTGGTGATTCTCTTGCTATGGC |
| | reading | TCGTCGTCTTGCTCGTTCTGTTCGTCGTCTTACTCGTCGTCGTGCTCATCGTCTTCTTCGTACTCGTCGTCTTCTTAAGCGTGAGGGTGTT |
| | frame | CTTCAGGCTGCTAATTTTGATGAGAATGGTCTTATTAAGTCTCTTCCTAATACTCCTTGGCAGCTTCGTGCTGCTGCTCTTGATCGTAAGC |
| | for | TTACTCCTCTTGAGTGGTCTGCTGTTCTTCTTCATCTTATTAAGCATCGTGGTTATCTTTCTCAGCGTAAGAATGAGGGTGAGACTGCTGA |
| | Nme1Cas9 | TAAGGAGCTTGGTGCTCTTCTTAAGGGTGTTGCTGGTAATGCTCATGCTCTTCAGACTGGTGATTTTCGTACTCCTGCTGAGCTTGCTCTT |
| | cleavase | AATAAGTTTGAGAAGGAGTCTGGTCATATTCGTAATCAGCGTTCTGATTATTCTCATACTTTTTCTCGTAAGGATCTTCAGGCTGAGCTTA |
| | | TTCTTCTTTTTGAGAAGCAGAAGGAGTTTGGTAATCCTCATGTTTCTGGTGGTCTTAAGGAGGGTATTGAGACTCTTCTTATGACTCAGCG |
| | | TCCTGCTCTTTCTGGTGATGCTGTTCAGAAGATGCTTGGTCATTGTACTTTTGAGCCTGCTGAGCCTAAGGCTGCTAAGAATACTTATACT |
| | | GCTGAGCGTTTTATTTGGCTTACTAAGCTTAATAATCTTCGTATTCTTGAGCAGGGTTCTGAGCGTCCTCTTACTGATACTGAGCGTGCTA |
| | | CTCTTATGGATGAGCCTTATCGTAAGTCTAAGCTTACTTATGCTCAGGCTCGTAAGCTTCTTGGTCTTGAGGATACTGCTTTTTTTAAGGG |
| | | TCTTCGTTATGGTAAGGATAATGCTGAGGCTTCTACTCTTATGGAGATGAAGGCTTATCATGCTATTTCTCGTGCTCTTGAGAAGGAGGGT |
| | | CTTAAGGATAAGAAGTCTCCTCTTAATCTTTCTCCTGAGCTTCAGGATGAGATTGGTACTGCTTTTTCTCTTTTTAAGACTGATGAGGATA |
| | | TTACTGGTCGTCTTAAGGATCGTATTCAGCCTGAGATTCTTGAGGCTCTTCTTAAGCATATTTCTTTTGATAAGTTTGTTCAGATTTCTCT |
| | | TAAGGCTCTTCGTCGTATTGTTCCTCTTATGGAGCAGGGTAAGCGTTATGATGAGGCTTGTGCTGAGATTTATGGTGATCATTATGGTAAG |
| | | AAGAATACTGAGGAGAAGATTTATCTTCCTCCTATTCCTGCTGATGAGATTCGTAATCCTGTTGTTCTTCGTGCTCTTTCTCAGGCTCGTA |
| | | AGGTTATTAATGGTGTTGTTCGTCGTTATGGTTCTCCTGCTCGTATTCATATTGAGACTGCTCGTGAGGTTGGTAAGTCTTTTAAGGATCG |
| | | TAAGGAGATTGAGAAGCGTCAGGAGGAGAATCGTAAGGATCGTGAGAAGGCTGCTGCTAAGTTTCGTGAGTATTTTCCTAATTTTGTTGGT |
| | | GAGCCTAAGTCTAAGGATATTCTTAAGCTTCGTCTTTATGAGCAGCAGCATGGTAAGTGTCTTTATTCTGGTAAGGAGATTAATCTTGGTC |
| | | GTCTTAATGAGAAGGGTTATGTTGAGATTGATCATGCTCTTCCTTTTTCTCGTACTTGGGATGATTCTTTTAATAATAAGGTTCTTGTTCT |
| | | TGGTTCTGAGAATCAGAATAAGGGTAATCAGACTCCTTATGAGTATTTTAATGGTAAGGATAATTCTCGTGAGTGGCAGGAGTTTAAGGCT |
| | | CGTGTTGAGACTTCTCGTTTTCCTCGTTCTAAGAAGCAGCGTATTCTTCTTCAGAAGTTTGATGAGGATGGTTTTAAGGAGCGTAATCTTA |
| | | ATGATACTCGTTATGTTAATCGTTTTCTTTGTCAGTTTGTTGCTGATCGTATGCGTCTTACTGGTAAGGGTAAGAAGCGTGTTTTTGCTTC |
| | | TAATGGTCAGATTACTAATCTTCTTCGTGGTTTTTGGGGTCTTCGTAAGGTTCGTGCTGAGAATGATCGTCATCATGCTCTTGATGCTGTT |
| | | GTTGTTGCTTGTTCTACTGTTGCTATGCAGCAGAAGATTACTCGTTTTGTTCGTTATAAGGAGATGAATGCTTTTGATGGTAAGACTATTG |
| | | ATAAGGAGACTGGTGAGGTTCTTCATCAGAAGACTCATTTTCCTCAGCCTTGGGAGTTTTTTGCTCAGGAGGTTATGATTCGTGTTTTTGG |
| | | TAAGCCTGATGGTAAGCCTGAGTTTGAGGAGGCTGATACTCTTGAGAAGCTTCGTACTCTTCTTGCTGAGAAGCTTTCTTCTCGTCCTGAG |
| | | GCTGTTCATGAGTATGTTACTCCTCTTTTTGTTTCTCGTGCTCCTAATCGTAAGATGTCTGGTCAGGGTCATATGGAGACTGTTAAGTCTG |
| | | CTAAGCGTCTTGATGAGGGTGTTTCTGTTCTTCGTGTTCCTCTTACTCAGCTTAAGCTTAAGGATCTTGAGAAGATGGTTAATCGTGAGCG |
| | | TGAGCCTAAGCTTTATGAGGCTCTTAAGGCTCGTCTTGAGGCTCATAAGGATGATCCTGCTAAGGCTTTTGCTGAGCCTTTTTATAAGTAT |
| | | GATAAGGCTGGTAATCGTACTCAGCAGGTTAAGGCTGTTCGTGTTGAGCAGGTTCAGAAGACTGGTGTTTGGGTTCGTAATCATAATGGTA |
| | | TTGCTGATAATGCTACTATGGTTCGTGTTGATGTTTTTGAGAAGGGTGATAAGTATTATCTTGTTCCTATTTATTCTTGGCAGGTTGCTAA |
| | | GGGTATTCTTCCTGATCGTGCTGTTGTTCAGGGTAAGGATGAGGAGGATTGGCAGCTTATTGATGATTCTTTTAATTTTAAGTTTTCTCTT |
| | | CATCCTAATGATCTTGTTGAGGTTATTACTAAGAAGGCTCGTATGTTTGGTTATTTTGCTTCTTGTCATCGTGGTACTGGTAATATTAATA |
| | | TTCGTATTCATGATCTTGATCATAAGATTGGTAAGAATGGTATTCTTGAGGGTATTGGTGTTAAGACTGCTCTTTCTTTTCAGAAGTATCA |
| | | GATTGATGAGCTTGGTAAGGAGATTCGTCCTTGTCGTCTTAAGAAGCGTCCTCCTGTTCGTUGA |
| 125 | Exemplary | ATGGCCGCCTTCAAGCCCAACTCCATCAACTACATCCTGGGCCTGGACATCGGCATCGCCTCCGTGGGCTGGGCCATGGTGGAGATCGACG |
| | open | AGGAGGAGAACCCCATCCGGCTGATCGACCTGGGCGTGCGGGTGTTCGAGCGGGCCGAGGTGCCCAAGACCGGCGACTCCCTGGCCATGGC |
| | reading | CCGGCGGCTGGCCCGGTCCGTGCGGCGGCTGACCCGGCGGCGGGCCCACCGGCTGCTGCGGACCCGGCGGCTGCTGAAGCGGGAGGGCGTG |
| | frame | CTGCAGGCCGCCAACTTCGACGAGAACGGCCTGATCAAGTCCCTGCCCAACACCCCCTGGCAGCTGCGGGCCGCCGCCCTGGACCGGAAGC |
| | for | TGACCCCCCTGGAGTGGTCCGCCGTGCTGCTGCACCTGATCAAGCACCGGGGCTACCTGTCCCAGCGGAAGAACGAGGGCGAGACCGCCGA |
| | Nme1Cas9 | CAAGGAGCTGGGCGCCCTGCTGAAGGGCGTGGCCGGCAACGCCCACGCCCTGCAGACCGGCGACTTCCGGACCCCCGCCGAGCTGGCCCTG |
| | cleavase | AACAAGTTCGAGAAGGAGTCCGGCCACATCCGGAACCAGCGGTCCGACTACTCCCACACCTTCTCCCGGAAGGACCTGCAGGCCGAGCTGA |
| | | TCCTGCTGTTCGAGAAGCAGAAGGAGTTCGGCAACCCCCACGTGTCCGGCGGCCTGAAGGAGGGCATCGAGACCCTGCTGATGACCCAGCG |
| | | GCCCGCCCTGTCCGGCGACGCCGTGCAGAAGATGCTGGGCCACTGCACCTTCGAGCCCGCCGAGCCCAAGGCCGCCAAGAACACCTACACC |
| | | GCCGAGCGGTTCATCTGGCTGACCAAGCTGAACAACCTGCGGATCCTGGAGCAGGGCTCCGAGCGGCCCCTGACCGACACCGAGCGGGCCA |
| | | CCCTGATGGACGAGCCCTACCGGAAGTCCAAGCTGACCTACGCCCAGGCCCGGAAGCTGCTGGGCCTGGAGGACACCGCCTTCTTCAAGGG |
| | | CCTGCGGTACGGCAAGGACAACGCCGAGGCCTCCACCCTGATGGAGATGAAGGCCTACCACGCCATCTCCCGGGCCCTGGAGAAGGAGGGC |
| | | CTGAAGGACAAGAAGTCCCCCCTGAACCTGTCCCCCGAGCTGCAGGACGAGATCGGCACCGCCTTCTCCCTGTTCAAGACCGACGAGGACA |
| | | TCACCGGCCGGCTGAAGGACCGGATCCAGCCCGAGATCCTGGAGGCCCTGCTGAAGCACATCTCCTTCGACAAGTTCGTGCAGATCTCCCT |
| | | GAAGGCCCTGCGGCGGATCGTGCCCCTGATGGAGCAGGGCAAGCGGTACGACGAGGCCTGCGCCGAGATCTACGGCGACCACTACGGCAAG |
| | | AAGAACACCGAGGAGAAGATCTACCTGCCCCCCATCCCCGCCGACGAGATCCGGAACCCCGTGGTGCTGCGGGCCCTGTCCCAGGCCCGGA |
| | | AGGTGATCAACGGCGTGGTGCGGCGGTACGGCTCCCCCGCCCGGATCCACATCGAGACCGCCCGGGAGGTGGGCAAGTCCTTCAAGGACCG |
| | | GAAGGAGATCGAGAAGCGGCAGGAGGAGAACCGGAAGGACCGGGAGAAGGCCGCCGCCAAGTTCCGGGAGTACTTCCCCAACTTCGTGGGC |
| | | GAGCCCAAGTCCAAGGACATCCTGAAGCTGCGGCTGTACGAGCAGCAGCACGGCAAGTGCCTGTACTCCGGCAAGGAGATCAACCTGGGCC |
| | | GGCTGAACGAGAAGGGCTACGTGGAGATCGACCACGCCCTGCCCTTCTCCCGGACCTGGGACGACTCCTTCAACAACAAGGTGCTGGTGCT |
| | | GGGCTCCGAGAACCAGAACAAGGGCAACCAGACCCCCTACGAGTACTTCAACGGCAAGGACAACTCCCGGGAGTGGCAGGAGTTCAAGGCC |
| | | CGGGTGGAGACCTCCCGGTTCCCCCGGTCCAAGAAGCAGCGGATCCTGCTGCAGAAGTTCGACGAGGACGGCTTCAAGGAGCGGAACCTGA |
| | | ACGACACCCGGTACGTGAACCGGTTCCTGTGCCAGTTCGTGGCCGACCGGATGCGGCTGACCGGCAAGGGCAAGAAGCGGGTGTTCGCCTC |
| | | CAACGGCCAGATCACCAACCTGCTGCGGGGCTTCTGGGGCCTGCGGAAGGTGCGGGCCGAGAACGACCGGCACCACGCCCTGGACGCCGTG |
| | | GTGGTGGCCTGCTCCACCGTGGCCATGCAGCAGAAGATCACCCGGTTCGTGCGGTACAAGGAGATGAACGCCTTCGACGGCAAGACCATCG |
| | | ACAAGGAGACCGGCGAGGTGCTGCACCAGAAGACCCACTTCCCCCAGCCCTGGGAGTTCTTCGCCCAGGAGGTGATGATCCGGGTGTTCGG |
| | | CAAGCCCGACGGCAAGCCCGAGTTCGAGGAGGCCGACACCCTGGAGAAGCTGCGGACCCTGCTGGCCGAGAAGCTGTCCTCCCGGCCCGAG |
| | | GCCGTGCACGAGTACGTGACCCCCCTGTTCGTGTCCCGGGCCCCCAACCGGAAGATGTCCGGCCAGGGCCACATGGAGACCGTGAAGTCCG |
| | | CCAAGCGGCTGGACGAGGGCGTGTCCGTGCTGCGGGTGCCCCTGACCCAGCTGAAGCTGAAGGACCTGGAGAAGATGGTGAACCGGGAGCG |
| | | GGAGCCCAAGCTGTACGAGGCCCTGAAGGCCCGGCTGGAGGCCCACAAGGACGACCCCGCCAAGGCCTTCGCCGAGCCCTTCTACAAGTAC |
| | | GACAAGGCCGGCAACCGGACCCAGCAGGTGAAGGCCGTGCGGGTGGAGCAGGTGCAGAAGACCGGCGTGTGGGTGCGGAACCACAACGGCA |
| | | TCGCCGACAACGCCACCATGGTGCGGGTGGACGTGTTCGAGAAGGGCGACAAGTACTACCTGGTGCCCATCTACTCCTGGCAGGTGGCCAA |
| | | GGGCATCCTGCCCGACCGGGCCGTGGTGCAGGGCAAGGACGAGGAGGACTGGCAGCTGATCGACGACTCCTTCAACTTCAAGTTCTCCCTG |
| | | CACCCCAACGACCTGGTGGAGGTGATCACCAAGAAGGCCCGGATGTTCGGCTACTTCGCCTCCTGCCACCGGGGCACCGGCAACATCAACA |
| | | TCCGGATCCACGACCTGGACCACAAGATCGGCAAGAACGGCATCCTGGAGGGCATCGGCGTGAAGACCGCCCTGTCCTTCCAGAAGTACCA |
| | | GATCGACGAGCTGGGCAAGGAGATCCGGCCCTGCCGGCTGAAGAAGCGGCCCCCCGTGCGGUGA |
| 126 | Exemplary | ATGGCAGCATTCAAACCAAACTCAATCAACTACATCCTAGGACTAGACATCGGAATCGCATCAGTAGGATGAGCAATGGTAGAAATCGACG |
| | open | AAGAAGAAAACCCAATCCGACTAATCGACCTAGGAGTACGAGTATTCGAACGAGCAGAAGTACCAAAAACAGGAGACTCACTAGCAATGGC |
| | reading | ACGACGACTAGCACGATCAGTACGACGACTAACACGACGACGAGCACACCGACTACTACGAACACGACGACTACTAAAACGAGAAGGAGTA |
| | frame | CTACAAGCAGCAAACTTCGACGAAAACGGACTAATCAAATCACTACCAAACACACCATGACAACTACGAGCAGCAGCACTAGACCGAAAAC |
| | for | TAACACCACTAGAATGATCAGCAGTACTACTACACCTAATCAAACACCGAGGATACCTATCACAACGAAAAAACGAAGGAGAAACAGCAGA |
| | Nme1Cas9 | CAAAGAACTAGGAGCACTACTAAAAGGAGTAGCAGGAAACGCACACGCACTACAAACAGGAGACTTCCGAACACCAGCAGAACTAGCACTA |
| | cleavase | AACAAATTCGAAAAAGAATCAGGACACATCCGAAACCAACGATCAGACTACTCACACACATTCTCACGAAAAGACCTACAAGCAGAACTAA |
| | | TCCTACTATTCGAAAAACAAAAAGAATTCGGAAACCCACACGTATCAGGAGGACTAAAAGAAGGAATCGAAACACTACTAATGACACAACG |
| | | ACCAGCACTATCAGGAGACGCAGTACAAAAAATGCTAGGACACTGCACATTCGAACCAGCAGAACCAAAAGCAGCAAAAAACACATACACA |
| | | GCAGAACGATTCATCTGACTAACAAAACTAAACAACCTACGAATCCTAGAACAAGGATCAGAACGACCACTAACAGACACAGAACGAGCAA |
| | | CACTAATGGACGAACCATACCGAAAATCAAAACTAACATACGCACAAGCACGAAAACTACTAGGACTAGAAGACACAGCATTCTTCAAAGG |
| | | ACTACGATACGGAAAAGACAACGCAGAAGCATCAACACTAATGGAAATGAAAGCATACCACGCAATCTCACGAGCACTAGAAAAAGAAGGA |
| | | CTAAAAGACAAAAAATCACCACTAAACCTATCACCAGAACTACAAGACGAAATCGGAACAGCATTCTCACTATTCAAAACAGACGAAGACA |
| | | TCACAGGACGACTAAAAGACCGAATCCAACCAGAAATCCTAGAAGCACTACTAAAACACATCTCATTCGACAAATTCGTACAAATCTCACT |
| | | AAAAGCACTACGACGAATCGTACCACTAATGGAACAAGGAAAACGATACGACGAAGCATGCGCAGAAATCTACGGAGACCACTACGGAAAA |
| | | AAAAACACAGAAGAAAAAATCTACCTACCACCAATCCCAGCAGACGAAATCCGAAACCCAGTAGTACTACGAGCACTATCACAAGCACGAA |
| | | AAGTAATCAACGGAGTAGTACGACGATACGGATCACCAGCACGAATCCACATCGAAACAGCACGAGAAGTAGGAAAATCATTCAAAGACCG |
| | | AAAAGAAATCGAAAAACGACAAGAAGAAAACCGAAAAGACCGAGAAAAAGCAGCAGCAAAATTCCGAGAATACTTCCCAAACTTCGTAGGA |
| | | GAACCAAAATCAAAAGACATCCTAAAACTACGACTATACGAACAACAACACGGAAAATGCCTATACTCAGGAAAAGAAATCAACCTAGGAC |
| | | GACTAAACGAAAAAGGATACGTAGAAATCGACCACGCACTACCATTCTCACGAACATGAGACGACTCATTCAACAACAAAGTACTAGTACT |
| | | AGGATCAGAAAACCAAAACAAAGGAAACCAAACACCATACGAATACTTCAACGGAAAAGACAACTCACGAGAATGACAAGAATTCAAAGCA |
| | | CGAGTAGAAACATCACGATTCCCACGATCAAAAAAACAACGAATCCTACTACAAAAATTCGACGAAGACGGATTCAAAGAACGAAACCTAA |
| | | ACGACACACGATACGTAAACCGATTCCTATGCCAATTCGTAGCAGACCGAATGCGACTAACAGGAAAAGGAAAAAAACGAGTATTCGCATC |
| | | AAACGGACAAATCACAAACCTACTACGAGGATTCTGAGGACTACGAAAAGTACGAGCAGAAAACGACCGACACCACGCACTAGACGCAGTA |
| | | GTAGTAGCATGCTCAACAGTAGCAATGCAACAAAAAATCACACGATTCGTACGATACAAAGAAATGAACGCATTCGACGGAAAAACAATCG |
| | | ACAAAGAAACAGGAGAAGTACTACACCAAAAAACACACTTCCCACAACCATGAGAATTCTTCGCACAAGAAGTAATGATCCGAGTATTCGG |
| | | AAAACCAGACGGAAAACCAGAATTCGAAGAAGCAGACACACTAGAAAAACTACGAACACTACTAGCAGAAAAACTATCATCACGACCAGAA |
| | | GCAGTACACGAATACGTAACACCACTATTCGTATCACGAGCACCAAACCGAAAAATGTCAGGACAAGGACACATGGAAACAGTAAAATCAG |
| | | CAAAACGACTAGACGAAGGAGTATCAGTACTACGAGTACCACTAACACAACTAAAACTAAAAGACCTAGAAAAAATGGTAAACCGAGAACG |
| | | AGAACCAAAACTATACGAAGCACTAAAAGCACGACTAGAAGCACACAAAGACGACCCAGCAAAAGCATTCGCAGAACCATTCTACAAATAC |
| | | GACAAAGCAGGAAACCGAACACAACAAGTAAAAGCAGTACGAGTAGAACAAGTACAAAAAACAGGAGTATGAGTACGAAACCACAACGGAA |
| | | TCGCAGACAACGCAACAATGGTACGAGTAGACGTATTCGAAAAAGGAGACAAATACTACCTAGTACCAATCTACTCATGACAAGTAGCAAA |
| | | AGGAATCCTACCAGACCGAGCAGTAGTACAAGGAAAAGACGAAGAAGACTGACAACTAATCGACGACTCATTCAACTTCAAATTCTCACTA |
| | | CACCCAAACGACCTAGTAGAAGTAATCACAAAAAAAGCACGAATGTTCGGATACTTCGCATCATGCCACCGAGGAACAGGAAACATCAACA |
| | | TCCGAATCCACGACCTAGACCACAAAATCGGAAAAAACGGAATCCTAGAAGGAATCGGAGTAAAAACAGCACTATCATTCCAAAAATACCA |
| | | AATCGACGAACTAGGAAAAGAAATCCGACCATGCCGACTAAAAAAACGACCACCAGTACGAUAA |
| 127 | Exemplary | MAAFKPNPINYILGLDIGIASVGWAMVEIDEEENPIRLIDLGVRVFERAEVPKTGDSLAMARRLARSVRRLTRRRAHRLLRARRLLKREGV |
| | amino | LQAADFDENGLIKSLPNTPWQLRAAALDRKLTPLEWSAVLLHLIKHRGYLSQRKNEGETADKELGALLKGVADNAHALQTGDFRTPAELAL |
| | acid | NKFEKECGHIRNQRGDYSHTFSRKDLQAELNLLFEKQKEFGNPHVSGGLKEGIETLLMTQRPALSGDAVQKMLGHCTFEPAEPKAAKNTYT |
| | sequence of | AERFIWLTKLNNLRILEQGSERPLTDTERATLMDEPYRKSKLTYAQARKLLSLEDTAFFKGLRYGKDNAEASTLMEMKAYHTISRALEKEG |
| | Nme3Cas9 | LKDKKSPLNLSPELQDEIGTAFSLFKTDEDITGRLKDRIQPEILEALLKHISFDKFVQISLKALRRIVPLMEQGKRYDEACAEIYGDHYGK |
| | cleavase | KNTEEKIYLPPIPADEIRNPVVLRALSQARKVINGVVRRYGSPARIHIETAREVGKSFKDRKEIEKRQEENRKDREKAAAKFREYFPNFVG |
| | | EPKSKDILKLRLYEQQHGKCLYSGKEINLGRLNEKGYVEIDHALPFSRTWDDSFNNKVLVLGSENQNKGNQTPYEYFNGKDNSREWQEFKA |
| | | RVETSRFPRSKKQRILLQKFDEDGFKERNLNDTRYVNRFLCQFVADRMRLTGKGKKRVFASNGQITNLLRGFWGLRKVRAENDRHHALDAV |
| | | VVACSTVAMQQKITRFVRYKEMNAFDGKTIDKETGEVLHQKTHFPQPWEFFAQEVMIRVFGKPDGKPEFEEADTPEKLRTLLAEKLSSRPE |
| | | AVHEYVTPLFVSRAPNRKMSGQGHMETVKSAKRLDEGVSVLRVPLTQLKLKDLEKMVNREREPKLYEALKARLEAHKDDPAKAFAEPFYKY |
| | | DKAGNRTQQVKAVRVEQVQKTGVWVRNHNGIADNATMVRVDVFEKGDKYYLVPIYSWQVAKGILPDRAVVAYADEEDWTVIDESFRFKFVL |
| | | YSNDLIKVQLKKDSFLGYFSGLDRATGAISLREHDLEKSKGKDGMHRIGVKTALSFQKYQIDEMGKEIRPCRLKKRPPVR |
| 128 | Exemplary | GCTGCTTTTAAGCCTAATCCTATTAATTATATTCTTGGTCTTGATATTGGTATTGCTTCTGTTGGTTGGGCTATGGTTGAGATTGATGAGG |
| | coding | AGGAGAATCCTATTCGTCTTATTGATCTTGGTGTTCGTGTTTTTGAGCGTGCTGAGGTTCCTAAGACTGGTGATTCTCTTGCTATGGCTCG |
| | sequence | TCGTCTTGCTCGTTCTGTTCGTCGTCTTACTCGTCGTCGTGCTCATCGTCTTCTTCGTGCTCGTCGTCTTCTTAAGCGTGAGGGTGTTCTT |
| | encoding | CAGGCTGCTGATTTTGATGAGAATGGTCTTATTAAGTCTCTTCCTAATACTCCTTGGCAGCTTCGTGCTGCTGCTCTTGATCGTAAGCTTA |
| | Nme3Cas9 | CTCCTCTTGAGTGGTCTGCTGTTCTTCTTCATCTTATTAAGCATCGTGGTTATCTTTCTCAGCGTAAGAATGAGGGTGAGACTGCTGATAA |
| | cleavase | GGAGCTTGGTGCTCTTCTTAAGGGTGTTGCTGATAATGCTCATGCTCTTCAGACTGGTGATTTTCGTACTCCTGCTGAGCTTGCTCTTAAT |
| | | AAGTTTGAGAAGGAGTGTGGTCATATTCGTAATCAGCGTGGTGATTATTCTCATACTTTTTCTCGTAAGGATCTTCAGGCTGAGCTTAATC |
| | | TTCTTTTTGAGAAGCAGAAGGAGTTTGGTAATCCTCATGTTTCTGGTGGTCTTAAGGAGGGTATTGAGACTCTTCTTATGACTCAGCGTCC |
| | | TGCTCTTTCTGGTGATGCTGTTCAGAAGATGCTTGGTCATTGTACTTTTGAGCCTGCTGAGCCTAAGGCTGCTAAGAATACTTATACTGCT |
| | | GAGCGTTTTATTTGGCTTACTAAGCTTAATAATCTTCGTATTCTTGAGCAGGGTTCTGAGCGTCCTCTTACTGATACTGAGCGTGCTACTC |
| | | TTATGGATGAGCCTTATCGTAAGTCTAAGCTTACTTATGCTCAGGCTCGTAAGCTTCTTTCTCTTGAGGATACTGCTTTTTTTAAGGGTCT |
| | | TCGTTATGGTAAGGATAATGCTGAGGCTTCTACTCTTATGGAGATGAAGGCTTATCATACTATTTCTCGTGCTCTTGAGAAGGAGGGTCTT |
| | | AAGGATAAGAAGTCTCCTCTTAATCTTTCTCCTGAGCTTCAGGATGAGATTGGTACTGCTTTTTCTCTTTTTAAGACTGATGAGGATATTA |
| | | CTGGTCGTCTTAAGGATCGTATTCAGCCTGAGATTCTTGAGGCTCTTCTTAAGCATATTTCTTTTGATAAGTTTGTTCAGATTTCTCTTAA |
| | | GGCTCTTCGTCGTATTGTTCCTCTTATGGAGCAGGGTAAGCGTTATGATGAGGCTTGTGCTGAGATTTATGGTGATCATTATGGTAAGAAG |
| | | AATACTGAGGAGAAGATTTATCTTCCTCCTATTCCTGCTGATGAGATTCGTAATCCTGTTGTTCTTCGTGCTCTTTCTCAGGCTCGTAAGG |
| | | TTATTAATGGTGTTGTTCGTCGTTATGGTTCTCCTGCTCGTATTCATATTGAGACTGCTCGTGAGGTTGGTAAGTCTTTTAAGGATCGTAA |
| | | GGAGATTGAGAAGCGTCAGGAGGAGAATCGTAAGGATCGTGAGAAGGCTGCTGCTAAGTTTCGTGAGTATTTTCCTAATTTTGTTGGTGAG |
| | | CCTAAGTCTAAGGATATTCTTAAGCTTCGTCTTTATGAGCAGCAGCATGGTAAGTGTCTTTATTCTGGTAAGGAGATTAATCTTGGTCGTC |
| | | TTAATGAGAAGGGTTATGTTGAGATTGATCATGCTCTTCCTTTTTCTCGTACTTGGGATGATTCTTTTAATAATAAGGTTCTTGTTCTTGG |
| | | TTCTGAGAATCAGAATAAGGGTAATCAGACTCCTTATGAGTATTTTAATGGTAAGGATAATTCTCGTGAGTGGCAGGAGTTTAAGGCTCGT |
| | | GTTGAGACTTCTCGTTTTCCTCGTTCTAAGAAGCAGCGTATTCTTCTTCAGAAGTTTGATGAGGATGGTTTTAAGGAGCGTAATCTTAATG |
| | | ATACTCGTTATGTTAATCGTTTTCTTTGTCAGTTTGTTGCTGATCGTATGCGTCTTACTGGTAAGGGTAAGAAGCGTGTTTTTGCTTCTAA |
| | | TGGTCAGATTACTAATCTTCTTCGTGGTTTTTGGGGTCTTCGTAAGGTTCGTGCTGAGAATGATCGTCATCATGCTCTTGATGCTGTTGTT |
| | | GTTGCTTGTTCTACTGTTGCTATGCAGCAGAAGATTACTCGTTTTGTTCGTTATAAGGAGATGAATGCTTTTGATGGTAAGACTATTGATA |
| | | AGGAGACTGGTGAGGTTCTTCATCAGAAGACTCATTTTCCTCAGCCTTGGGAGTTTTTTGCTCAGGAGGTTATGATTCGTGTTTTTGGTAA |
| | | GCCTGATGGTAAGCCTGAGTTTGAGGAGGCTGATACTCCTGAGAAGCTTCGTACTCTTCTTGCTGAGAAGCTTTCTTCTCGTCCTGAGGCT |
| | | GTTCATGAGTATGTTACTCCTCTTTTTGTTTCTCGTGCTCCTAATCGTAAGATGTCTGGTCAGGGTCATATGGAGACTGTTAAGTCTGCTA |
| | | AGCGTCTTGATGAGGGTGTTTCTGTTCTTCGTGTTCCTCTTACTCAGCTTAAGCTTAAGGATCTTGAGAAGATGGTTAATCGTGAGCGTGA |
| | | GCCTAAGCTTTATGAGGCTCTTAAGGCTCGTCTTGAGGCTCATAAGGATGATCCTGCTAAGGCTTTTGCTGAGCCTTTTTATAAGTATGAT |
| | | AAGGCTGGTAATCGTACTCAGCAGGTTAAGGCTGTTCGTGTTGAGCAGGTTCAGAAGACTGGTGTTTGGGTTCGTAATCATAATGGTATTG |
| | | CTGATAATGCTACTATGGTTCGTGTTGATGTTTTTGAGAAGGGTGATAAGTATTATCTTGTTCCTATTTATTCTTGGCAGGTTGCTAAGGG |
| | | TATTCTTCCTGATCGTGCTGTTGTTGCTTATGCTGATGAGGAGGATTGGACTGTTATTGATGAGTCTTTTCGTTTTAAGTTTGTTCTTTAT |
| | | TCTAATGATCTTATTAAGGTTCAGCTTAAGAAGGATTCTTTTCTTGGTTATTTTTCTGGTCTTGATCGTGCTACTGGTGCTATTTCTCTTC |
| | | GTGAGCATGATCTTGAGAAGTCTAAGGGTAAGGATGGTATGCATCGTATTGGTGTTAAGACTGCTCTTTCTTTTCAGAAGTATCAGATTGA |
| | | TGAGATGGGTAAGGAGATTCGTCCTTGTCGTCTTAAGAAGCGTCCTCCTGTTCGT |
| 129 | Exemplary | GCCGCCTTCAAGCCCAACCCCATCAACTACATCCTGGGCCTGGACATCGGCATCGCCTCCGTGGGCTGGGCCATGGTGGAGATCGACGAGG |
| | coding | AGGAGAACCCCATCCGGCTGATCGACCTGGGCGTGCGGGTGTTCGAGCGGGCCGAGGTGCCCAAGACCGGCGACTCCCTGGCCATGGCCCG |
| | sequence | GCGGCTGGCCCGGTCCGTGCGGCGGCTGACCCGGCGGCGGGCCCACCGGCTGCTGCGGGCCCGGCGGCTGCTGAAGCGGGAGGGCGTGCTG |
| | encoding | CAGGCCGCCGACTTCGACGAGAACGGCCTGATCAAGTCCCTGCCCAACACCCCCTGGCAGCTGCGGGCCGCCGCCCTGGACCGGAAGCTGA |
| | Nme3Cas9 | CCCCCCTGGAGTGGTCCGCCGTGCTGCTGCACCTGATCAAGCACCGGGGCTACCTGTCCCAGCGGAAGAACGAGGGCGAGACCGCCGACAA |
| | cleavase | GGAGCTGGGCGCCCTGCTGAAGGGCGTGGCCGACAACGCCCACGCCCTGCAGACCGGCGACTTCCGGACCCCCGCCGAGCTGGCCCTGAAC |
| | | AAGTTCGAGAAGGAGTGCGGCCACATCCGGAACCAGCGGGGCGACTACTCCCACACCTTCTCCCGGAAGGACCTGCAGGCCGAGCTGAACC |
| | | TGCTGTTCGAGAAGCAGAAGGAGTTCGGCAACCCCCACGTGTCCGGCGGCCTGAAGGAGGGCATCGAGACCCTGCTGATGACCCAGCGGCC |
| | | CGCCCTGTCCGGCGACGCCGTGCAGAAGATGCTGGGCCACTGCACCTTCGAGCCCGCCGAGCCCAAGGCCGCCAAGAACACCTACACCGCC |
| | | GAGCGGTTCATCTGGCTGACCAAGCTGAACAACCTGCGGATCCTGGAGCAGGGCTCCGAGCGGCCCCTGACCGACACCGAGCGGGCCACCC |
| | | TGATGGACGAGCCCTACCGGAAGTCCAAGCTGACCTACGCCCAGGCCCGGAAGCTGCTGTCCCTGGAGGACACCGCCTTCTTCAAGGGCCT |
| | | GCGGTACGGCAAGGACAACGCCGAGGCCTCCACCCTGATGGAGATGAAGGCCTACCACACCATCTCCCGGGCCCTGGAGAAGGAGGGCCTG |
| | | AAGGACAAGAAGTCCCCCCTGAACCTGTCCCCCGAGCTGCAGGACGAGATCGGCACCGCCTTCTCCCTGTTCAAGACCGACGAGGACATCA |
| | | CCGGCCGGCTGAAGGACCGGATCCAGCCCGAGATCCTGGAGGCCCTGCTGAAGCACATCTCCTTCGACAAGTTCGTGCAGATCTCCCTGAA |
| | | GGCCCTGCGGCGGATCGTGCCCCTGATGGAGCAGGGCAAGCGGTACGACGAGGCCTGCGCCGAGATCTACGGCGACCACTACGGCAAGAAG |
| | | AACACCGAGGAGAAGATCTACCTGCCCCCCATCCCCGCCGACGAGATCCGGAACCCCGTGGTGCTGCGGGCCCTGTCCCAGGCCCGGAAGG |
| | | TGATCAACGGCGTGGTGCGGCGGTACGGCTCCCCCGCCCGGATCCACATCGAGACCGCCCGGGAGGTGGGCAAGTCCTTCAAGGACCGGAA |
| | | GGAGATCGAGAAGCGGCAGGAGGAGAACCGGAAGGACCGGGAGAAGGCCGCCGCCAAGTTCCGGGAGTACTTCCCCAACTTCGTGGGCGAG |
| | | CCCAAGTCCAAGGACATCCTGAAGCTGCGGCTGTACGAGCAGCAGCACGGCAAGTGCCTGTACTCCGGCAAGGAGATCAACCTGGGCCGGC |
| | | TGAACGAGAAGGGCTACGTGGAGATCGACCACGCCCTGCCCTTCTCCCGGACCTGGGACGACTCCTTCAACAACAAGGTGCTGGTGCTGGG |
| | | CTCCGAGAACCAGAACAAGGGCAACCAGACCCCCTACGAGTACTTCAACGGCAAGGACAACTCCCGGGAGTGGCAGGAGTTCAAGGCCCGG |
| | | GTGGAGACCTCCCGGTTCCCCCGGTCCAAGAAGCAGCGGATCCTGCTGCAGAAGTTCGACGAGGACGGCTTCAAGGAGCGGAACCTGAACG |
| | | ACACCCGGTACGTGAACCGGTTCCTGTGCCAGTTCGTGGCCGACCGGATGCGGCTGACCGGCAAGGGCAAGAAGCGGGTGTTCGCCTCCAA |
| | | CGGCCAGATCACCAACCTGCTGCGGGGCTTCTGGGGCCTGCGGAAGGTGCGGGCCGAGAACGACCGGCACCACGCCCTGGACGCCGTGGTG |
| | | GTGGCCTGCTCCACCGTGGCCATGCAGCAGAAGATCACCCGGTTCGTGCGGTACAAGGAGATGAACGCCTTCGACGGCAAGACCATCGACA |
| | | AGGAGACCGGCGAGGTGCTGCACCAGAAGACCCACTTCCCCCAGCCCTGGGAGTTCTTCGCCCAGGAGGTGATGATCCGGGTGTTCGGCAA |
| | | GCCCGACGGCAAGCCCGAGTTCGAGGAGGCCGACACCCCCGAGAAGCTGCGGACCCTGCTGGCCGAGAAGCTGTCCTCCCGGCCCGAGGCC |
| | | GTGCACGAGTACGTGACCCCCCTGTTCGTGTCCCGGGCCCCCAACCGGAAGATGTCCGGCCAGGGCCACATGGAGACCGTGAAGTCCGCCA |
| | | AGCGGCTGGACGAGGGCGTGTCCGTGCTGCGGGTGCCCCTGACCCAGCTGAAGCTGAAGGACCTGGAGAAGATGGTGAACCGGGAGCGGGA |
| | | GCCCAAGCTGTACGAGGCCCTGAAGGCCCGGCTGGAGGCCCACAAGGACGACCCCGCCAAGGCCTTCGCCGAGCCCTTCTACAAGTACGAC |
| | | AAGGCCGGCAACCGGACCCAGCAGGTGAAGGCCGTGCGGGTGGAGCAGGTGCAGAAGACCGGCGTGTGGGTGCGGAACCACAACGGCATCG |
| | | CCGACAACGCCACCATGGTGCGGGTGGACGTGTTCGAGAAGGGCGACAAGTACTACCTGGTGCCCATCTACTCCTGGCAGGTGGCCAAGGG |
| | | CATCCTGCCCGACCGGGCCGTGGTGGCCTACGCCGACGAGGAGGACTGGACCGTGATCGACGAGTCCTTCCGGTTCAAGTTCGTGCTGTAC |
| | | TCCAACGACCTGATCAAGGTGCAGCTGAAGAAGGACTCCTTCCTGGGCTACTTCTCCGGCCTGGACCGGGCCACCGGCGCCATCTCCCTGC |
| | | GGGAGCACGACCTGGAGAAGTCCAAGGGCAAGGACGGCATGCACCGGATCGGCGTGAAGACCGCCCTGTCCTTCCAGAAGTACCAGATCGA |
| | | CGAGATGGGCAAGGAGATCCGGCCCTGCCGGCTGAAGAAGCGGCCCCCCGTGCGG |
| 130 | Exemplary | GCAGCATTCAAACCAAACCCAATCAACTACATCCTAGGACTAGACATCGGAATCGCATCAGTAGGATGAGCAATGGTAGAAATCGACGAAG |
| | coding | AAGAAAACCCAATCCGACTAATCGACCTAGGAGTACGAGTATTCGAACGAGCAGAAGTACCAAAAACAGGAGACTCACTAGCAATGGCACG |
| | sequence | ACGACTAGCACGATCAGTACGACGACTAACACGACGACGAGCACACCGACTACTACGAGCACGACGACTACTAAAACGAGAAGGAGTACTA |
| | encoding | CAAGCAGCAGACTTCGACGAAAACGGACTAATCAAATCACTACCAAACACACCATGACAACTACGAGCAGCAGCACTAGACCGAAAACTAA |
| | Nme3Cas9 | CACCACTAGAATGATCAGCAGTACTACTACACCTAATCAAACACCGAGGATACCTATCACAACGAAAAAACGAAGGAGAAACAGCAGACAA |
| | cleavase | AGAACTAGGAGCACTACTAAAAGGAGTAGCAGACAACGCACACGCACTACAAACAGGAGACTTCCGAACACCAGCAGAACTAGCACTAAAC |
| | | AAATTCGAAAAAGAATGCGGACACATCCGAAACCAACGAGGAGACTACTCACACACATTCTCACGAAAAGACCTACAAGCAGAACTAAACC |
| | | TACTATTCGAAAAACAAAAAGAATTCGGAAACCCACACGTATCAGGAGGACTAAAAGAAGGAATCGAAACACTACTAATGACACAACGACC |
| | | AGCACTATCAGGAGACGCAGTACAAAAAATGCTAGGACACTGCACATTCGAACCAGCAGAACCAAAAGCAGCAAAAAACACATACACAGCA |
| | | GAACGATTCATCTGACTAACAAAACTAAACAACCTACGAATCCTAGAACAAGGATCAGAACGACCACTAACAGACACAGAACGAGCAACAC |
| | | TAATGGACGAACCATACCGAAAATCAAAACTAACATACGCACAAGCACGAAAACTACTATCACTAGAAGACACAGCATTCTTCAAAGGACT |
| | | ACGATACGGAAAAGACAACGCAGAAGCATCAACACTAATGGAAATGAAAGCATACCACACAATCTCACGAGCACTAGAAAAAGAAGGACTA |
| | | AAAGACAAAAAATCACCACTAAACCTATCACCAGAACTACAAGACGAAATCGGAACAGCATTCTCACTATTCAAAACAGACGAAGACATCA |
| | | CAGGACGACTAAAAGACCGAATCCAACCAGAAATCCTAGAAGCACTACTAAAACACATCTCATTCGACAAATTCGTACAAATCTCACTAAA |
| | | AGCACTACGACGAATCGTACCACTAATGGAACAAGGAAAACGATACGACGAAGCATGCGCAGAAATCTACGGAGACCACTACGGAAAAAAA |
| | | AACACAGAAGAAAAAATCTACCTACCACCAATCCCAGCAGACGAAATCCGAAACCCAGTAGTACTACGAGCACTATCACAAGCACGAAAAG |
| | | TAATCAACGGAGTAGTACGACGATACGGATCACCAGCACGAATCCACATCGAAACAGCACGAGAAGTAGGAAAATCATTCAAAGACCGAAA |
| | | AGAAATCGAAAAACGACAAGAAGAAAACCGAAAAGACCGAGAAAAAGCAGCAGCAAAATTCCGAGAATACTTCCCAAACTTCGTAGGAGAA |
| | | CCAAAATCAAAAGACATCCTAAAACTACGACTATACGAACAACAACACGGAAAATGCCTATACTCAGGAAAAGAAATCAACCTAGGACGAC |
| | | TAAACGAAAAAGGATACGTAGAAATCGACCACGCACTACCATTCTCACGAACATGAGACGACTCATTCAACAACAAAGTACTAGTACTAGG |
| | | ATCAGAAAACCAAAACAAAGGAAACCAAACACCATACGAATACTTCAACGGAAAAGACAACTCACGAGAATGACAAGAATTCAAAGCACGA |
| | | GTAGAAACATCACGATTCCCACGATCAAAAAAACAACGAATCCTACTACAAAAATTCGACGAAGACGGATTCAAAGAACGAAACCTAAACG |
| | | ACACACGATACGTAAACCGATTCCTATGCCAATTCGTAGCAGACCGAATGCGACTAACAGGAAAAGGAAAAAAACGAGTATTCGCATCAAA |
| | | CGGACAAATCACAAACCTACTACGAGGATTCTGAGGACTACGAAAAGTACGAGCAGAAAACGACCGACACCACGCACTAGACGCAGTAGTA |
| | | GTAGCATGCTCAACAGTAGCAATGCAACAAAAAATCACACGATTCGTACGATACAAAGAAATGAACGCATTCGACGGAAAAACAATCGACA |
| | | AAGAAACAGGAGAAGTACTACACCAAAAAACACACTTCCCACAACCATGAGAATTCTTCGCACAAGAAGTAATGATCCGAGTATTCGGAAA |
| | | ACCAGACGGAAAACCAGAATTCGAAGAAGCAGACACACCAGAAAAACTACGAACACTACTAGCAGAAAAACTATCATCACGACCAGAAGCA |
| | | GTACACGAATACGTAACACCACTATTCGTATCACGAGCACCAAACCGAAAAATGTCAGGACAAGGACACATGGAAACAGTAAAATCAGCAA |
| | | AACGACTAGACGAAGGAGTATCAGTACTACGAGTACCACTAACACAACTAAAACTAAAAGACCTAGAAAAAATGGTAAACCGAGAACGAGA |
| | | ACCAAAACTATACGAAGCACTAAAAGCACGACTAGAAGCACACAAAGACGACCCAGCAAAAGCATTCGCAGAACCATTCTACAAATACGAC |
| | | AAAGCAGGAAACCGAACACAACAAGTAAAAGCAGTACGAGTAGAACAAGTACAAAAAACAGGAGTATGAGTACGAAACCACAACGGAATCG |
| | | CAGACAACGCAACAATGGTACGAGTAGACGTATTCGAAAAAGGAGACAAATACTACCTAGTACCAATCTACTCATGACAAGTAGCAAAAGG |
| | | AATCCTACCAGACCGAGCAGTAGTAGCATACGCAGACGAAGAAGACTGAACAGTAATCGACGAATCATTCCGATTCAAATTCGTACTATAC |
| | | TCAAACGACCTAATCAAAGTACAACTAAAAAAAGACTCATTCCTAGGATACTTCTCAGGACTAGACCGAGCAACAGGAGCAATCTCACTAC |
| | | GAGAACACGACCTAGAAAAATCAAAAGGAAAAGACGGAATGCACCGAATCGGAGTAAAAACAGCACTATCATTCCAAAAATACCAAATCGA |
| | | CGAAATGGGAAAAGAAATCCGACCATGCCGACTAAAAAAACGACCACCAGTACGA |
| 131 | Exemplary | ATGGCTGCTTTTAAGCCTAATCCTATTAATTATATTCTTGGTCTTGATATTGGTATTGCTTCTGTTGGTTGGGCTATGGTTGAGATTGATG |
| | open | AGGAGGAGAATCCTATTCGTCTTATTGATCTTGGTGTTCGTGTTTTTGAGCGTGCTGAGGTTCCTAAGACTGGTGATTCTCTTGCTATGGC |
| | reading | TCGTCGTCTTGCTCGTTCTGTTCGTCGTCTTACTCGTCGTCGTGCTCATCGTCTTCTTCGTGCTCGTCGTCTTCTTAAGCGTGAGGGTGTT |
| | frame for | CTTCAGGCTGCTGATTTTGATGAGAATGGTCTTATTAAGTCTCTTCCTAATACTCCTTGGCAGCTTCGTGCTGCTGCTCTTGATCGTAAGC |
| | Nme3Cas9 | TTACTCCTCTTGAGTGGTCTGCTGTTCTTCTTCATCTTATTAAGCATCGTGGTTATCTTTCTCAGCGTAAGAATGAGGGTGAGACTGCTGA |
| | cleavase | TAAGGAGCTTGGTGCTCTTCTTAAGGGTGTTGCTGATAATGCTCATGCTCTTCAGACTGGTGATTTTCGTACTCCTGCTGAGCTTGCTCTT |
| | | AATAAGTTTGAGAAGGAGTGTGGTCATATTCGTAATCAGCGTGGTGATTATTCTCATACTTTTTCTCGTAAGGATCTTCAGGCTGAGCTTA |
| | | ATCTTCTTTTTGAGAAGCAGAAGGAGTTTGGTAATCCTCATGTTTCTGGTGGTCTTAAGGAGGGTATTGAGACTCTTCTTATGACTCAGCG |
| | | TCCTGCTCTTTCTGGTGATGCTGTTCAGAAGATGCTTGGTCATTGTACTTTTGAGCCTGCTGAGCCTAAGGCTGCTAAGAATACTTATACT |
| | | GCTGAGCGTTTTATTTGGCTTACTAAGCTTAATAATCTTCGTATTCTTGAGCAGGGTTCTGAGCGTCCTCTTACTGATACTGAGCGTGCTA |
| | | CTCTTATGGATGAGCCTTATCGTAAGTCTAAGCTTACTTATGCTCAGGCTCGTAAGCTTCTTTCTCTTGAGGATACTGCTTTTTTTAAGGG |
| | | TCTTCGTTATGGTAAGGATAATGCTGAGGCTTCTACTCTTATGGAGATGAAGGCTTATCATACTATTTCTCGTGCTCTTGAGAAGGAGGGT |
| | | CTTAAGGATAAGAAGTCTCCTCTTAATCTTTCTCCTGAGCTTCAGGATGAGATTGGTACTGCTTTTTCTCTTTTTAAGACTGATGAGGATA |
| | | TTACTGGTCGTCTTAAGGATCGTATTCAGCCTGAGATTCTTGAGGCTCTTCTTAAGCATATTTCTTTTGATAAGTTTGTTCAGATTTCTCT |
| | | TAAGGCTCTTCGTCGTATTGTTCCTCTTATGGAGCAGGGTAAGCGTTATGATGAGGCTTGTGCTGAGATTTATGGTGATCATTATGGTAAG |
| | | AAGAATACTGAGGAGAAGATTTATCTTCCTCCTATTCCTGCTGATGAGATTCGTAATCCTGTTGTTCTTCGTGCTCTTTCTCAGGCTCGTA |
| | | AGGTTATTAATGGTGTTGTTCGTCGTTATGGTTCTCCTGCTCGTATTCATATTGAGACTGCTCGTGAGGTTGGTAAGTCTTTTAAGGATCG |
| | | TAAGGAGATTGAGAAGCGTCAGGAGGAGAATCGTAAGGATCGTGAGAAGGCTGCTGCTAAGTTTCGTGAGTATTTTCCTAATTTTGTTGGT |
| | | GAGCCTAAGTCTAAGGATATTCTTAAGCTTCGTCTTTATGAGCAGCAGCATGGTAAGTGTCTTTATTCTGGTAAGGAGATTAATCTTGGTC |
| | | GTCTTAATGAGAAGGGTTATGTTGAGATTGATCATGCTCTTCCTTTTTCTCGTACTTGGGATGATTCTTTTAATAATAAGGTTCTTGTTCT |
| | | TGGTTCTGAGAATCAGAATAAGGGTAATCAGACTCCTTATGAGTATTTTAATGGTAAGGATAATTCTCGTGAGTGGCAGGAGTTTAAGGCT |
| | | CGTGTTGAGACTTCTCGTTTTCCTCGTTCTAAGAAGCAGCGTATTCTTCTTCAGAAGTTTGATGAGGATGGTTTTAAGGAGCGTAATCTTA |
| | | ATGATACTCGTTATGTTAATCGTTTTCTTTGTCAGTTTGTTGCTGATCGTATGCGTCTTACTGGTAAGGGTAAGAAGCGTGTTTTTGCTTC |
| | | TAATGGTCAGATTACTAATCTTCTTCGTGGTTTTTGGGGTCTTCGTAAGGTTCGTGCTGAGAATGATCGTCATCATGCTCTTGATGCTGTT |
| | | GTTGTTGCTTGTTCTACTGTTGCTATGCAGCAGAAGATTACTCGTTTTGTTCGTTATAAGGAGATGAATGCTTTTGATGGTAAGACTATTG |
| | | ATAAGGAGACTGGTGAGGTTCTTCATCAGAAGACTCATTTTCCTCAGCCTTGGGAGTTTTTTGCTCAGGAGGTTATGATTCGTGTTTTTGG |
| | | TAAGCCTGATGGTAAGCCTGAGTTTGAGGAGGCTGATACTCCTGAGAAGCTTCGTACTCTTCTTGCTGAGAAGCTTTCTTCTCGTCCTGAG |
| | | GCTGTTCATGAGTATGTTACTCCTCTTTTTGTTTCTCGTGCTCCTAATCGTAAGATGTCTGGTCAGGGTCATATGGAGACTGTTAAGTCTG |
| | | CTAAGCGTCTTGATGAGGGTGTTTCTGTTCTTCGTGTTCCTCTTACTCAGCTTAAGCTTAAGGATCTTGAGAAGATGGTTAATCGTGAGCG |
| | | TGAGCCTAAGCTTTATGAGGCTCTTAAGGCTCGTCTTGAGGCTCATAAGGATGATCCTGCTAAGGCTTTTGCTGAGCCTTTTTATAAGTAT |
| | | GATAAGGCTGGTAATCGTACTCAGCAGGTTAAGGCTGTTCGTGTTGAGCAGGTTCAGAAGACTGGTGTTTGGGTTCGTAATCATAATGGTA |
| | | TTGCTGATAATGCTACTATGGTTCGTGTTGATGTTTTTGAGAAGGGTGATAAGTATTATCTTGTTCCTATTTATTCTTGGCAGGTTGCTAA |
| | | GGGTATTCTTCCTGATCGTGCTGTTGTTGCTTATGCTGATGAGGAGGATTGGACTGTTATTGATGAGTCTTTTCGTTTTAAGTTTGTTCTT |
| | | TATTCTAATGATCTTATTAAGGTTCAGCTTAAGAAGGATTCTTTTCTTGGTTATTTTTCTGGTCTTGATCGTGCTACTGGTGCTATTTCTC |
| | | TTCGTGAGCATGATCTTGAGAAGTCTAAGGGTAAGGATGGTATGCATCGTATTGGTGTTAAGACTGCTCTTTCTTTTCAGAAGTATCAGAT |
| | | TGATGAGATGGGTAAGGAGATTCGTCCTTGTCGTCTTAAGAAGCGTCCTCCTGTTCGTUGA |
| 132 | Exemplary | ATGGCCGCCTTCAAGCCCAACCCCATCAACTACATCCTGGGCCTGGACATCGGCATCGCCTCCGTGGGCTGGGCCATGGTGGAGATCGACG |
| | open | AGGAGGAGAACCCCATCCGGCTGATCGACCTGGGCGTGCGGGTGTTCGAGCGGGCCGAGGTGCCCAAGACCGGCGACTCCCTGGCCATGGC |
| | reading | CCGGCGGCTGGCCCGGTCCGTGCGGCGGCTGACCCGGCGGCGGGCCCACCGGCTGCTGCGGGCCCGGCGGCTGCTGAAGCGGGAGGGCGTG |
| | frame | CTGCAGGCCGCCGACTTCGACGAGAACGGCCTGATCAAGTCCCTGCCCAACACCCCCTGGCAGCTGCGGGCCGCCGCCCTGGACCGGAAGC |
| | for | TGACCCCCCTGGAGTGGTCCGCCGTGCTGCTGCACCTGATCAAGCACCGGGGCTACCTGTCCCAGCGGAAGAACGAGGGCGAGACCGCCGA |
| | Nme3Cas9 | CAAGGAGCTGGGCGCCCTGCTGAAGGGCGTGGCCGACAACGCCCACGCCCTGCAGACCGGCGACTTCCGGACCCCCGCCGAGCTGGCCCTG |
| | cleavase | AACAAGTTCGAGAAGGAGTGCGGCCACATCCGGAACCAGCGGGGCGACTACTCCCACACCTTCTCCCGGAAGGACCTGCAGGCCGAGCTGA |
| | | ACCTGCTGTTCGAGAAGCAGAAGGAGTTCGGCAACCCCCACGTGTCCGGCGGCCTGAAGGAGGGCATCGAGACCCTGCTGATGACCCAGCG |
| | | GCCCGCCCTGTCCGGCGACGCCGTGCAGAAGATGCTGGGCCACTGCACCTTCGAGCCCGCCGAGCCCAAGGCCGCCAAGAACACCTACACC |
| | | GCCGAGCGGTTCATCTGGCTGACCAAGCTGAACAACCTGCGGATCCTGGAGCAGGGCTCCGAGCGGCCCCTGACCGACACCGAGCGGGCCA |
| | | CCCTGATGGACGAGCCCTACCGGAAGTCCAAGCTGACCTACGCCCAGGCCCGGAAGCTGCTGTCCCTGGAGGACACCGCCTTCTTCAAGGG |
| | | CCTGCGGTACGGCAAGGACAACGCCGAGGCCTCCACCCTGATGGAGATGAAGGCCTACCACACCATCTCCCGGGCCCTGGAGAAGGAGGGC |
| | | CTGAAGGACAAGAAGTCCCCCCTGAACCTGTCCCCCGAGCTGCAGGACGAGATCGGCACCGCCTTCTCCCTGTTCAAGACCGACGAGGACA |
| | | TCACCGGCCGGCTGAAGGACCGGATCCAGCCCGAGATCCTGGAGGCCCTGCTGAAGCACATCTCCTTCGACAAGTTCGTGCAGATCTCCCT |
| | | GAAGGCCCTGCGGCGGATCGTGCCCCTGATGGAGCAGGGCAAGCGGTACGACGAGGCCTGCGCCGAGATCTACGGCGACCACTACGGCAAG |
| | | AAGAACACCGAGGAGAAGATCTACCTGCCCCCCATCCCCGCCGACGAGATCCGGAACCCCGTGGTGCTGCGGGCCCTGTCCCAGGCCCGGA |
| | | AGGTGATCAACGGCGTGGTGCGGCGGTACGGCTCCCCCGCCCGGATCCACATCGAGACCGCCCGGGAGGTGGGCAAGTCCTTCAAGGACCG |
| | | GAAGGAGATCGAGAAGCGGCAGGAGGAGAACCGGAAGGACCGGGAGAAGGCCGCCGCCAAGTTCCGGGAGTACTTCCCCAACTTCGTGGGC |
| | | GAGCCCAAGTCCAAGGACATCCTGAAGCTGCGGCTGTACGAGCAGCAGCACGGCAAGTGCCTGTACTCCGGCAAGGAGATCAACCTGGGCC |
| | | GGCTGAACGAGAAGGGCTACGTGGAGATCGACCACGCCCTGCCCTTCTCCCGGACCTGGGACGACTCCTTCAACAACAAGGTGCTGGTGCT |
| | | GGGCTCCGAGAACCAGAACAAGGGCAACCAGACCCCCTACGAGTACTTCAACGGCAAGGACAACTCCCGGGAGTGGCAGGAGTTCAAGGCC |
| | | CGGGTGGAGACCTCCCGGTTCCCCCGGTCCAAGAAGCAGCGGATCCTGCTGCAGAAGTTCGACGAGGACGGCTTCAAGGAGCGGAACCTGA |
| | | ACGACACCCGGTACGTGAACCGGTTCCTGTGCCAGTTCGTGGCCGACCGGATGCGGCTGACCGGCAAGGGCAAGAAGCGGGTGTTCGCCTC |
| | | CAACGGCCAGATCACCAACCTGCTGCGGGGCTTCTGGGGCCTGCGGAAGGTGCGGGCCGAGAACGACCGGCACCACGCCCTGGACGCCGTG |
| | | GTGGTGGCCTGCTCCACCGTGGCCATGCAGCAGAAGATCACCCGGTTCGTGCGGTACAAGGAGATGAACGCCTTCGACGGCAAGACCATCG |
| | | ACAAGGAGACCGGCGAGGTGCTGCACCAGAAGACCCACTTCCCCCAGCCCTGGGAGTTCTTCGCCCAGGAGGTGATGATCCGGGTGTTCGG |
| | | CAAGCCCGACGGCAAGCCCGAGTTCGAGGAGGCCGACACCCCCGAGAAGCTGCGGACCCTGCTGGCCGAGAAGCTGTCCTCCCGGCCCGAG |
| | | GCCGTGCACGAGTACGTGACCCCCCTGTTCGTGTCCCGGGCCCCCAACCGGAAGATGTCCGGCCAGGGCCACATGGAGACCGTGAAGTCCG |
| | | CCAAGCGGCTGGACGAGGGCGTGTCCGTGCTGCGGGTGCCCCTGACCCAGCTGAAGCTGAAGGACCTGGAGAAGATGGTGAACCGGGAGCG |
| | | GGAGCCCAAGCTGTACGAGGCCCTGAAGGCCCGGCTGGAGGCCCACAAGGACGACCCCGCCAAGGCCTTCGCCGAGCCCTTCTACAAGTAC |
| | | GACAAGGCCGGCAACCGGACCCAGCAGGTGAAGGCCGTGCGGGTGGAGCAGGTGCAGAAGACCGGCGTGTGGGTGCGGAACCACAACGGCA |
| | | TCGCCGACAACGCCACCATGGTGCGGGTGGACGTGTTCGAGAAGGGCGACAAGTACTACCTGGTGCCCATCTACTCCTGGCAGGTGGCCAA |
| | | GGGCATCCTGCCCGACCGGGCCGTGGTGGCCTACGCCGACGAGGAGGACTGGACCGTGATCGACGAGTCCTTCCGGTTCAAGTTCGTGCTG |
| | | TACTCCAACGACCTGATCAAGGTGCAGCTGAAGAAGGACTCCTTCCTGGGCTACTTCTCCGGCCTGGACCGGGCCACCGGCGCCATCTCCC |
| | | TGCGGGAGCACGACCTGGAGAAGTCCAAGGGCAAGGACGGCATGCACCGGATCGGCGTGAAGACCGCCCTGTCCTTCCAGAAGTACCAGAT |
| | | CGACGAGATGGGCAAGGAGATCCGGCCCTGCCGGCTGAAGAAGCGGCCCCCCGTGCGGUGA |
| 133 | Exemplary | ATGGCAGCATTCAAACCAAACCCAATCAACTACATCCTAGGACTAGACATCGGAATCGCATCAGTAGGATGAGCAATGGTAGAAATCGACG |
| | open | AAGAAGAAAACCCAATCCGACTAATCGACCTAGGAGTACGAGTATTCGAACGAGCAGAAGTACCAAAAACAGGAGACTCACTAGCAATGGC |
| | reading | ACGACGACTAGCACGATCAGTACGACGACTAACACGACGACGAGCACACCGACTACTACGAGCACGACGACTACTAAAACGAGAAGGAGTA |
| | frame | CTACAAGCAGCAGACTTCGACGAAAACGGACTAATCAAATCACTACCAAACACACCATGACAACTACGAGCAGCAGCACTAGACCGAAAAC |
| | for | TAACACCACTAGAATGATCAGCAGTACTACTACACCTAATCAAACACCGAGGATACCTATCACAACGAAAAAACGAAGGAGAAACAGCAGA |
| | Nme3Cas9 | CAAAGAACTAGGAGCACTACTAAAAGGAGTAGCAGACAACGCACACGCACTACAAACAGGAGACTTCCGAACACCAGCAGAACTAGCACTA |
| | cleavase | AACAAATTCGAAAAAGAATGCGGACACATCCGAAACCAACGAGGAGACTACTCACACACATTCTCACGAAAAGACCTACAAGCAGAACTAA |
| | | ACCTACTATTCGAAAAACAAAAAGAATTCGGAAACCCACACGTATCAGGAGGACTAAAAGAAGGAATCGAAACACTACTAATGACACAACG |
| | | ACCAGCACTATCAGGAGACGCAGTACAAAAAATGCTAGGACACTGCACATTCGAACCAGCAGAACCAAAAGCAGCAAAAAACACATACACA |
| | | GCAGAACGATTCATCTGACTAACAAAACTAAACAACCTACGAATCCTAGAACAAGGATCAGAACGACCACTAACAGACACAGAACGAGCAA |
| | | CACTAATGGACGAACCATACCGAAAATCAAAACTAACATACGCACAAGCACGAAAACTACTATCACTAGAAGACACAGCATTCTTCAAAGG |
| | | ACTACGATACGGAAAAGACAACGCAGAAGCATCAACACTAATGGAAATGAAAGCATACCACACAATCTCACGAGCACTAGAAAAAGAAGGA |
| | | CTAAAAGACAAAAAATCACCACTAAACCTATCACCAGAACTACAAGACGAAATCGGAACAGCATTCTCACTATTCAAAACAGACGAAGACA |
| | | TCACAGGACGACTAAAAGACCGAATCCAACCAGAAATCCTAGAAGCACTACTAAAACACATCTCATTCGACAAATTCGTACAAATCTCACT |
| | | AAAAGCACTACGACGAATCGTACCACTAATGGAACAAGGAAAACGATACGACGAAGCATGCGCAGAAATCTACGGAGACCACTACGGAAAA |
| | | AAAAACACAGAAGAAAAAATCTACCTACCACCAATCCCAGCAGACGAAATCCGAAACCCAGTAGTACTACGAGCACTATCACAAGCACGAA |
| | | AAGTAATCAACGGAGTAGTACGACGATACGGATCACCAGCACGAATCCACATCGAAACAGCACGAGAAGTAGGAAAATCATTCAAAGACCG |
| | | AAAAGAAATCGAAAAACGACAAGAAGAAAACCGAAAAGACCGAGAAAAAGCAGCAGCAAAATTCCGAGAATACTTCCCAAACTTCGTAGGA |
| | | GAACCAAAATCAAAAGACATCCTAAAACTACGACTATACGAACAACAACACGGAAAATGCCTATACTCAGGAAAAGAAATCAACCTAGGAC |
| | | GACTAAACGAAAAAGGATACGTAGAAATCGACCACGCACTACCATTCTCACGAACATGAGACGACTCATTCAACAACAAAGTACTAGTACT |
| | | AGGATCAGAAAACCAAAACAAAGGAAACCAAACACCATACGAATACTTCAACGGAAAAGACAACTCACGAGAATGACAAGAATTCAAAGCA |
| | | CGAGTAGAAACATCACGATTCCCACGATCAAAAAAACAACGAATCCTACTACAAAAATTCGACGAAGACGGATTCAAAGAACGAAACCTAA |
| | | ACGACACACGATACGTAAACCGATTCCTATGCCAATTCGTAGCAGACCGAATGCGACTAACAGGAAAAGGAAAAAAACGAGTATTCGCATC |
| | | AAACGGACAAATCACAAACCTACTACGAGGATTCTGAGGACTACGAAAAGTACGAGCAGAAAACGACCGACACCACGCACTAGACGCAGTA |
| | | GTAGTAGCATGCTCAACAGTAGCAATGCAACAAAAAATCACACGATTCGTACGATACAAAGAAATGAACGCATTCGACGGAAAAACAATCG |
| | | ACAAAGAAACAGGAGAAGTACTACACCAAAAAACACACTTCCCACAACCATGAGAATTCTTCGCACAAGAAGTAATGATCCGAGTATTCGG |
| | | AAAACCAGACGGAAAACCAGAATTCGAAGAAGCAGACACACCAGAAAAACTACGAACACTACTAGCAGAAAAACTATCATCACGACCAGAA |
| | | GCAGTACACGAATACGTAACACCACTATTCGTATCACGAGCACCAAACCGAAAAATGTCAGGACAAGGACACATGGAAACAGTAAAATCAG |
| | | CAAAACGACTAGACGAAGGAGTATCAGTACTACGAGTACCACTAACACAACTAAAACTAAAAGACCTAGAAAAAATGGTAAACCGAGAACG |
| | | AGAACCAAAACTATACGAAGCACTAAAAGCACGACTAGAAGCACACAAAGACGACCCAGCAAAAGCATTCGCAGAACCATTCTACAAATAC |
| | | GACAAAGCAGGAAACCGAACACAACAAGTAAAAGCAGTACGAGTAGAACAAGTACAAAAAACAGGAGTATGAGTACGAAACCACAACGGAA |
| | | TCGCAGACAACGCAACAATGGTACGAGTAGACGTATTCGAAAAAGGAGACAAATACTACCTAGTACCAATCTACTCATGACAAGTAGCAAA |
| | | AGGAATCCTACCAGACCGAGCAGTAGTAGCATACGCAGACGAAGAAGACTGAACAGTAATCGACGAATCATTCCGATTCAAATTCGTACTA |
| | | TACTCAAACGACCTAATCAAAGTACAACTAAAAAAAGACTCATTCCTAGGATACTTCTCAGGACTAGACCGAGCAACAGGAGCAATCTCAC |
| | | TACGAGAACACGACCTAGAAAAATCAAAAGGAAAAGACGGAATGCACCGAATCGGAGTAAAAACAGCACTATCATTCCAAAAATACCAAAT |
| | | CGACGAAATGGGAAAAGAAATCCGACCATGCCGACTAAAAAAACGACCACCAGTACGAUAA |
| 134 | Exemplary | GGGAAGCUCAGAAUAAACGCUCAACUUUGGCCGGAUCUGCCACCAUGGACGGCUCCGGCGGCGGCUCCCCCAAGAAGAAGCGGAAGGUGGA |
| | coding | GGACAAGCGGCCCGCCGCCACCAAGAAGGCCGGCCAGGCCAAGAAGAAGAAGGGCGGCUCCGGGGGGGGCGCCGCCUUCAAGCCCAACCCC |
| | sequence | AUCAACUACAUCCUGGGCCUGGACAUCGGCAUCGCCUCCGUGGGCUGGGCCAUGGUGGAGAUCGACGAGGAGGAGAACCCCAUCCGGCUGA |
| | encoding | UCGACCUGGGCGUGCGGGUGUUCGAGCGGGCCGAGGUGCCCAAGACCGGCGACUCCCUGGCCAUGGCCCGGCGGCUGGCCCGGUCCGUGCG |
| | Nme2Cas9 | GCGGCUGACCCGGCGGCGGGCCCACCGGCUGCUGCGGGCCCGGCGGCUGCUGAAGCGGGAGGGCGUGCUGCAGGCCGCCGACUUCGACGAG |
| | (mRNA | AACGGCCUGAUCAAGUCCCUGCCCAACACCCCCUGGCAGCUGCGGGCCGCCGCCCUGGACCGGAAGCUGACCCCCCUGGAGUGGUCCGCCG |
| | AA) | UGCUGCUGCACCUGAUCAAGCACCGGGGCUACCUGUCCCAGCGGAAGAACGAGGGCGAGACCGCCGACAAGGAGCUGGGCGCCCUGCUGAA |
| | | GGGCGUGGCCAACAACGCCCACGCCCUGCAGACCGGCGACUUCCGGACCCCCGCCGAGCUGGCCCUGAACAAGUUCGAGAAGGAGUCCGGC |
| | | CACAUCCGGAACCAGCGGGGCGACUACUCCCACACCUUCUCCCGGAAGGACCUGCAGGCCGAGCUGAUCCUGCUGUUCGAGAAGCAGAAGG |
| | | AGUUCGGCAACCCCCACGUGUCCGGCGGCCUGAAGGAGGGCAUCGAGACCCUGCUGAUGACCCAGCGGCCCGCCCUGUCCGGCGACGCCGU |
| | | GCAGAAGAUGCUGGGCCACUGCACCUUCGAGCCCGCCGAGCCCAAGGCCGCCAAGAACACCUACACCGCCGAGCGGUUCAUCUGGCUGACC |
| | | AAGCUGAACAACCUGCGGAUCCUGGAGCAGGGCUCCGAGCGGCCCCUGACCGACACCGAGCGGGCCACCCUGAUGGACGAGCCCUACCGGA |
| | | AGUCCAAGCUGACCUACGCCCAGGCCCGGAAGCUGCUGGGCCUGGAGGACACCGCCUUCUUCAAGGGCCUGCGGUACGGCAAGGACAACGC |
| | | CGAGGCCUCCACCCUGAUGGAGAUGAAGGCCUACCACGCCAUCUCCCGGGCCCUGGAGAAGGAGGGCCUGAAGGACAAGAAGUCCCCCCUG |
| | | AACCUGUCCUCCGAGCUGCAGGACGAGAUCGGCACCGCCUUCUCCCUGUUCAAGACCGACGAGGACAUCACCGGCCGGCUGAAGGACCGGG |
| | | UGCAGCCCGAGAUCCUGGAGGCCCUGCUGAAGCACAUCUCCUUCGACAAGUUCGUGCAGAUCUCCCUGAAGGCCCUGCGGCGGAUCGUGCC |
| | | CCUGAUGGAGCAGGGCAAGCGGUACGACGAGGCCUGCGCCGAGAUCUACGGCGACCACUACGGCAAGAAGAACACCGAGGAGAAGAUCUAC |
| | | CUGCCCCCCAUCCCCGCCGACGAGAUCCGGAACCCCGUGGUGCUGCGGGCCCUGUCCCAGGCCCGGAAGGUGAUCAACGGCGUGGUGCGGC |
| | | GGUACGGCUCCCCCGCCCGGAUCCACAUCGAGACCGCCCGGGAGGUGGGCAAGUCCUUCAAGGACCGGAAGGAGAUCGAGAAGCGGCAGGA |
| | | GGAGAACCGGAAGGACCGGGAGAAGGCCGCCGCCAAGUUCCGGGAGUACUUCCCCAACUUCGUGGGCGAGCCCAAGUCCAAGGACAUCCUG |
| | | AAGCUGCGGCUGUACGAGCAGCAGCACGGCAAGUGCCUGUACUCCGGCAAGGAGAUCAACCUGGUGCGGCUGAACGAGAAGGGCUACGUGG |
| | | AGAUCGACCACGCCCUGCCCUUCUCCCGGACCUGGGACGACUCCUUCAACAACAAGGUGCUGGUGCUGGGCUCCGAGAACCAGAACAAGGG |
| | | CAACCAGACCCCCUACGAGUACUUCAACGGCAAGGACAACUCCCGGGAGUGGCAGGAGUUCAAGGCCCGGGUGGAGACCUCCCGGUUCCCC |
| | | CGGUCCAAGAAGCAGCGGAUCCUGCUGCAGAAGUUCGACGAGGACGGCUUCAAGGAGUGCAACCUGAACGACACCCGGUACGUGAACCGGU |
| | | UCCUGUGCCAGUUCGUGGCCGACCACAUCCUGCUGACCGGCAAGGGCAAGCGGCGGGUGUUCGCCUCCAACGGCCAGAUCACCAACCUGCU |
| | | GCGGGGCUUCUGGGGCCUGCGGAAGGUGCGGGCCGAGAACGACCGGCACCACGCCCUGGACGCCGUGGUGGUGGCCUGCUCCACCGUGGCC |
| | | AUGCAGCAGAAGAUCACCCGGUUCGUGCGGUACAAGGAGAUGAACGCCUUCGACGGCAAGACCAUCGACAAGGAGACCGGCAAGGUGCUGC |
| | | ACCAGAAGACCCACUUCCCCCAGCCCUGGGAGUUCUUCGCCCAGGAGGUGAUGAUCCGGGUGUUCGGCAAGCCCGACGGCAAGCCCGAGUU |
| | | CGAGGAGGCCGACACCCCCGAGAAGCUGCGGACCCUGCUGGCCGAGAAGCUGUCCUCCCGGCCCGAGGCCGUGCACGAGUACGUGACCCCC |
| | | CUGUUCGUGUCCCGGGCCCCCAACCGGAAGAUGUCCGGCGCCCACAAGGACACCCUGCGGUCCGCCAAGCGGUUCGUGAAGCACAACGAGA |
| | | AGAUCUCCGUGAAGCGGGUGUGGCUGACCGAGAUCAAGCUGGCCGACCUGGAGAACAUGGUGAACUACAAGAACGGCCGGGAGAUCGAGCU |
| | | GUACGAGGCCCUGAAGGCCCGGCUGGAGGCCUACGGCGGCAACGCCAAGCAGGCCUUCGACCCCAAGGACAACCCCUUCUACAAGAAGGGC |
| | | GGCCAGCUGGUGAAGGCCGUGCGGGUGGAGAAGACCCAGGAGUCCGGCGUGCUGCUGAACAAGAAGAACGCCUACACCAUCGCCGACAACG |
| | | GCGACAUGGUGCGGGUGGACGUGUUCUGCAAGGUGGACAAGAAGGGCAAGAACCAGUACUUCAUCGUGCCCAUCUACGCCUGGCAGGUGGC |
| | | CGAGAACAUCCUGCCCGACAUCGACUGCAAGGGCUACCGGAUCGACGACUCCUACACCUUCUGCUUCUCCCUGCACAAGUACGACCUGAUC |
| | | GCCUUCCAGAAGGACGAGAAGUCCAAGGUGGAGUUCGCCUACUACAUCAACUGCGACUCCUCCAACGGCCGGUUCUACCUGGCCUGGCACG |
| | | ACAAGGGCUCCAAGGAGCAGCAGUUCCGGAUCUCCACCCAGAACCUGGUGCUGAUCCAGAAGUACCAGGUGAACGAGCUGGGCAAGGAGAU |
| | | CCGGCCCUGCCGGCUGAAGAAGCGGCCCCCCGUGCGGUAGCUAGCACCAGCCUCAAGAACACCCGAAUGGAGUCUCUAAGCUACAUAAUAC |
| | | CAACUUACACUUUACAAAAUGUUGUCCCCCAAAAUGUAGCCAUUCGUAUCUGCUCCUAAUAAAAAGAAAGUUUCUUCACAUUCUCAAAAAA |
| | | AAAAAAAAAAAAAAAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAA |
| 135 | Exemplary | GGGUCCCGCAGUCGGCGUCCAGCGGCUCUGCUUGUUCGUGUGUGUGUCGUUGCAGGCCUUAUUCGGAUCCGCCACCAUGGCAGCAUUCAAG |
| | coding | CCGAACUCGAUCAACUACAUCCUGGGACUGGACAUCGGAAUCGCAUCGGUCGGAUGGGCAAUGGUCGAAAUCGACGAAGAAGAAAACCCGA |
| | sequence | UCAGACUGAUCGACCUGGGAGUCAGAGUCUUCGAAAGAGCAGAAGUCCCGAAGACAGGAGACUCGCUGGCAAUGGCAAGAAGACUGGCAAG |
| | encoding | AUCGGUCAGAAGACUGACAAGAAGAAGAGCACACAGACUGCUGAGAACAAGAAGACUGCUGAAGAGAGAAGGAGUCCUGCAGGCAGCAAAC |
| | Nme1Cas9 | UUCGACGAAAACGGACUGAUCAAGUCGCUGCCGAACACACCGUGGCAGCUGAGAGCAGCAGCACUGGACAGAAAGCUGACACCGCUGGAAU |
| | (mRNA | GGUCGGCAGUCCUGCUGCACCUGAUCAAGCACAGAGGAUACCUGUCGCAGAGAAAGAACGAAGGAGAAACAGCAGACAAGGAACUGGGAGC |
| | AB) | ACUGCUGAAGGGAGUCGCAGGAAACGCACACGCACUGCAGACAGGAGACUUCAGAACACCGGCAGAACUGGCACUGAACAAGUUCGAAAAG |
| | | GAAUCGGGACACAUCAGAAACCAGAGAUCGGACUACUCGCACACAUUCUCGAGAAAGGACCUGCAGGCAGAACUGAUCCUGCUGUUCGAAA |
| | | AGCAGAAGGAAUUCGGAAACCCGCACGUCUCGGGAGGACUGAAGGAAGGAAUCGAAACACUGCUGAUGACACAGAGACCGGCACUGUCGGG |
| | | AGACGCAGUCCAGAAGAUGCUGGGACACUGCACAUUCGAACCGGCAGAACCGAAGGCAGCAAAGAACACAUACACAGCAGAAAGAUUCAUC |
| | | UGGCUGACAAAGCUGAACAACCUGAGAAUCCUGGAACAGGGAUCGGAAAGACCGCUGACAGACACAGAAAGAGCAACACUGAUGGACGAAC |
| | | CGUACAGAAAGUCGAAGCUGACAUACGCACAGGCAAGAAAGCUGCUGGGACUGGAAGACACAGCAUUCUUCAAGGGACUGAGAUACGGAAA |
| | | GGACAACGCAGAAGCAUCGACACUGAUGGAAAUGAAGGCAUACCACGCAAUCUCGAGAGCACUGGAAAAGGAAGGACUGAAGGACAAGAAG |
| | | UCGCCGCUGAACCUGUCGCCGGAACUGCAGGACGAAAUCGGAACAGCAUUCUCGCUGUUCAAGACAGACGAAGACAUCACAGGAAGACUGA |
| | | AGGACAGAAUCCAGCCGGAAAUCCUGGAAGCACUGCUGAAGCACAUCUCGUUCGACAAGUUCGUCCAGAUCUCGCUGAAGGCACUGAGAAG |
| | | AAUCGUCCCGCUGAUGGAACAGGGAAAGAGAUACGACGAAGCAUGCGCAGAAAUCUACGGAGACCACUACGGAAAGAAGAACACAGAAGAA |
| | | AAGAUCUACCUGCCGCCGAUCCCGGCAGACGAAAUCAGAAACCCGGUCGUCCUGAGAGCACUGUCGCAGGCAAGAAAGGUCAUCAACGGAG |
| | | UCGUCAGAAGAUACGGAUCGCCGGCAAGAAUCCACAUCGAAACAGCAAGAGAAGUCGGAAAGUCGUUCAAGGACAGAAAGGAAAUCGAAAA |
| | | GAGACAGGAAGAAAACAGAAAGGACAGAGAAAAGGCAGCAGCAAAGUUCAGAGAAUACUUCCCGAACUUCGUCGGAGAACCGAAGUCGAAG |
| | | GACAUCCUGAAGCUGAGACUGUACGAACAGCAGCACGGAAAGUGCCUGUACUCGGGAAAGGAAAUCAACCUGGGAAGACUGAACGAAAAGG |
| | | GAUACGUCGAAAUCGACCACGCACUGCCGUUCUCGAGAACAUGGGACGACUCGUUCAACAACAAGGUCCUGGUCCUGGGAUCGGAAAACCA |
| | | GAACAAGGGAAACCAGACACCGUACGAAUACUUCAACGGAAAGGACAACUCGAGAGAAUGGCAGGAAUUCAAGGCAAGAGUCGAAACAUCG |
| | | AGAUUCCCGAGAUCGAAGAAGCAGAGAAUCCUGCUGCAGAAGUUCGACGAAGACGGAUUCAAGGAAAGAAACCUGAACGACACAAGAUACG |
| | | UCAACAGAUUCCUGUGCCAGUUCGUCGCAGACAGAAUGAGACUGACAGGAAAGGGAAAGAAGAGAGUCUUCGCAUCGAACGGACAGAUCAC |
| | | AAACCUGCUGAGAGGAUUCUGGGGACUGAGAAAGGUCAGAGCAGAAAACGACAGACACCACGCACUGGACGCAGUCGUCGUCGCAUGCUCG |
| | | ACAGUCGCAAUGCAGCAGAAGAUCACAAGAUUCGUCAGAUACAAGGAAAUGAACGCAUUCGACGGAAAGACAAUCGACAAGGAAACAGGAG |
| | | AAGUCCUGCACCAGAAGACACACUUCCCGCAGCCGUGGGAAUUCUUCGCACAGGAAGUCAUGAUCAGAGUCUUCGGAAAGCCGGACGGAAA |
| | | GCCGGAAUUCGAAGAAGCAGACACACUGGAAAAGCUGAGAACACUGCUGGCAGAAAAGCUGUCGUCGAGACCGGAAGCAGUCCACGAAUAC |
| | | GUCACACCGCUGUUCGUCUCGAGAGCACCGAACAGAAAGAUGUCGGGACAGGGACACAUGGAAACAGUCAAGUCGGCAAAGAGACUGGACG |
| | | AAGGAGUCUCGGUCCUGAGAGUCCCGCUGACACAGCUGAAGCUGAAGGACCUGGAAAAGAUGGUCAACAGAGAAAGAGAACCGAAGCUGUA |
| | | CGAAGCACUGAAGGCAAGACUGGAAGCACACAAGGACGACCCGGCAAAGGCAUUCGCAGAACCGUUCUACAAGUACGACAAGGCAGGAAAC |
| | | AGAACACAGCAGGUCAAGGCAGUCAGAGUCGAACAGGUCCAGAAGACAGGAGUCUGGGUCAGAAACCACAACGGAAUCGCAGACAACGCAA |
| | | CAAUGGUCAGAGUAGACGUCUUCGAAAAGGGAGACAAGUACUACCUGGUCCCGAUCUACUCGUGGCAGGUCGCAAAGGGAAUCCUGCCGGA |
| | | CAGAGCAGUCGUCCAGGGAAAGGACGAAGAAGACUGGCAGCUGAUCGACGACUCGUUCAACUUCAAGUUCUCGCUGCACCCGAACGACCUG |
| | | GUCGAAGUCAUCACAAAGAAGGCAAGAAUGUUCGGAUACUUCGCAUCGUGCCACAGAGGAACAGGAAACAUCAACAUCAGAAUCCACGACC |
| | | UGGACCACAAGAUCGGAAAGAACGGAAUCCUGGAAGGAAUCGGAGUCAAGACAGCACUGUCGUUCCAGAAGUACCAGAUCGACGAACUGGG |
| | | AAAGGAAAUCAGACCGUGCAGACUGAAGAAGAGACCGCCGGUCAGAUCCGGAAAGAGAACAGCAGACGGAUCGGAAUUCGAAUCGCCGAAG |
| | | AAGAAGAGAAAGGUCGAAUGAUAGCUAGCCAUCACAUUUAAAAGCAUCUCAGCCUACCAUGAGAAUAAGAGAAAGAAAAUGAAGAUCAAUA |
| | | GCUUAUUCAUCUCUUUUUCUUUUUCGUUGGUGUAAAGCCAACACCCUGUCUAAAAAACAUAAAUUUCUUUAAUCAUUUUGCCUCUUUUCUC |
| | | UGUGCUUCAAUUAAUAAAAAAUGGAAAGAACCUCGAGAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAUCUAG |
| 136 | Exemplary | MDGSGGGSPKKKRKVEDKRPAATKKAGQAKKKKGGSGGGAAFKPNPINYILGLDIGIASVGWAMVEIDEEENPIRLIDLGVRVFERAEVPK |
| | amino | TGDSLAMARRLARSVRRLTRRRAHRLLRARRLLKREGVLQAADFDENGLIKSLPNTPWQLRAAALDRKLTPLEWSAVLLHLI KHRGYLSQR |
| | acid | KNEGETADKELGALLKGVANNAHALQTGDFRTPAELALNKFEKESGHIRNQRGDYSHTFSRKDLQAELILLFEKQKEFGNPHVSGGLKEGI |
| | sequence | ETLLMTQRPALSGDAVQKMLGHCTFEPAEPKAAKNTYTAERFIWLTKLNNLRILEQGSERPLTDTERATLMDEPYRKSKLTYAQARKLLGL |
| | for | EDTAFFKGLRYGKDNAEASTLMEMKAYHAISRALEKEGLKDKKSPLNLSSELQDEIGTAFSLFKTDEDITGRLKDRVQPEILEALLKHISF |
| | Nme2Cas9 | DKFVQISLKALRRIVPLMEQGKRYDEACAEIYGDHYGKKNTEEKIYLPPIPADEIRNPVVLRALSQARKVINGVVRRYGSPARIHIETARE |
| | (mRNA | VGKSFKDRKEIEKRQEENRKDREKAAAKFREYFPNFVGEPKSKDILKLRLYEQQHGKCLYSGKEINLVRLNEKGYVEIDHALPFSRTWDDS |
| | AA | FNNKVLVLGSENQNKGNQTPYEYFNGKDNSREWQEFKARVETSRFPRSKKQRILLQKFDEDGFKECNLNDTRYVNRFLCQFVADHILLTGK |
| | amino | GKRRVFASNGQITNLLRGFWGLRKVRAENDRHHALDAVVVACSTVAMQQKITRFVRYKEMNAFDGKTIDKETGKVLHQKTHFPQPWEFFAQ |
| | acid) | EVMIRVFGKPDGKPEFEEADTPEKLRTLLAEKLSSRPEAVHEYVTPLFVSRAPNRKMSGAHKDTLRSAKRFVKHNEKI SVKRVWLTEIKLA |
| | | DLENMVNYKNGREIELYEALKARLEAYGGNAKQAFDPKDNPFYKKGGQLVKAVRVEKTQESGVLLNKKNAYTIADNGDMVRVDVFCKVDKK |
| | | GKNQYFIVPIYAWQVAENILPDIDCKGYRIDDSYTFCFSLHKYDLIAFQKDEKSKVEFAYYINCDSSNGRFYLAWHDKGSKEQQFRISTQN |
| | | LVLIQKYQVNELGKEIRPCRLKKRPPVR* |
| 137 | Exemplary | MAAFKPNSINYILGLDIGIASVGWAMVEIDEEENPIRLIDLGVRVFERAEVPKTGDSLAMARRLARSVRRLTRRRAHRLLRTRRLLKREGV |
| | amino | LQAANFDENGLIKSLPNTPWQLRAAALDRKLTPLEWSAVLLHLIKHRGYLSQRKNEGETADKELGALLKGVAGNAHALQTGDFRTPAELAL |
| | acid | NKFEKESGHIRNQRSDYSHTFSRKDLQAELILLFEKQKEFGNPHVSGGLKEGIETLLMTQRPALSGDAVQKMLGHCTFEPAEPKAAKNTYT |
| | sequence | AERFIWLTKLNNLRILEQGSERPLTDTERATLMDEPYRKSKLTYAQARKLLGLEDTAFFKGLRYGKDNAEASTLMEMKAYHAISRALEKEG |
| | for | LKDKKSPLNLSPELQDEIGTAFSLFKTDEDITGRLKDRIQPEILEALLKHISFDKFVQISLKALRRIVPLMEQGKRYDEACAEIYGDHYGK |
| | Nme1Cas9 | KNTEEKIYLPPIPADEIRNPVVLRALSQARKVINGVVRRYGSPARIHIETAREVGKSFKDRKEIEKRQEENRKDREKAAAKFREYFPNFVG |
| | (mRNA | EPKSKDILKLRLYEQQHGKCLYSGKEINLGRLNEKGYVEIDHALPFSRTWDDSFNNKVLVLGSENQNKGNQTPYEYFNGKDNSREWQEFKA |
| | AB | RVETSRFPRSKKQRILLQKFDEDGFKERNLNDTRYVNRFLCQFVADRMRLTGKGKKRVFASNGQITNLLRGFWGLRKVRAENDRHHALDAV |
| | amino | VVACSTVAMQQKITRFVRYKEMNAFDGKTIDKETGEVLHQKTHFPQPWEFFAQEVMIRVFGKPDGKPEFEEADTLEKLRTLLAEKLSSRPE |
| | acid) | AVHEYVTPLFVSRAPNRKMSGQGHMETVKSAKRLDEGVSVLRVPLTQLKLKDLEKMVNREREPKLYEALKARLEAHKDDPAKAFAEPFYKY |
| | | DKAGNRTQQVKAVRVEQVQKTGVWVRNHNGIADNATMVRVDVFEKGDKYYLVPIYSWQVAKGILPDRAVVQGKDEEDWQLIDDSFNFKFSL |
| | | HPNDLVEVITKKARMFGYFASCHRGTGNINIRIHDLDHKIGKNGILEGIGVKTALSFQKYQIDELGKEIRPCRLKKRPPVRSGKRTADGSE |
| | | FESPKKKRKVE* |
| 138 | Exemplary | augGACGGCUCCGGCGGCGGCUCCCCCAAGAAGAAGCGGAAGGUGGAGGACAAGCGGCCCGCCGCCACCAAGAAGGCCGGCCAGGCCAAGA |
| | open | AGAAGAAGGGCGGCUCCGGCGGCGGCgccgccuucaagcccaaccccaucaacuacauccugggccuggacaucggcaucgccuccguggg |
| | reading | cugggccaugguggagaucgacgaggaggagaaccccauccggcugaucgaccugggcgugcggguguucgagcgggccgaggugcccaag |
| | frame | accggcgacucccuggccauggcccggcggcuggcccgguccgugcggcggcugacccggcggcgcggcccaccggcugcuggggcccggc |
| | for | ggcugcugaagcgggagggcgugcugcaggccgccgacuucgacgagaacggccugaucaagucccugcccaacacccccuggcagcugcg |
| | Nme2Cas9 | ggccgccgcccuggaccggaagcugaccccccuggagugguccgccgugcugcugcaccugaucaagcaccggggcuaccugucccagcgg |
| | (mRNA | aagaacgagggcgagaccgccgacaaggagcugggcgcccugcugaagggcguggccaacaacgcccacgcccugcagaccggcgacuucc |
| | AA | ggacccccgccgagcuggcccugaacaaguucgagaaggaguccggccacauccggaaccaggggcgcgacuacucccacaccuucucccg |
| | ORF) | gaaggaccugcaggccgagcugauccugcuguucgagaagcagaaggaguucggcaacccccacguguccgcgggccugaaggagggcauc |
| | | gagacccugcugaugacccagcggcccgcccuguccggcgacgccgugcagaagaugcugggccacugcaccuucgagcccgccgagccca |
| | | aggccgccaagaacaccuacaccgccgagcgguucaucuggcugaccaagcugaacaaccugcggauccuggagcagggcuccgagcggcc |
| | | ccugaccgacaccgagcgggccacccugauggacgagcccuaccggaaguccaagcugaccuacgcccaggcccggaagcugcugggccug |
| | | gaggacaccgccuucuucaagggccugcgguacggcaaggacaacgccgaggccuccacccugauggagaugaaggccuaccacgccaucu |
| | | cccgggcccuggagaaggagggccugaaggacaagaaguccccccugaaccuguccuccgagcugcaggacgagaucggcaccgccuucuc |
| | | ccuguucaagaccgacgaggacaucaccggccggcugaaggaccgggugcagcccgagauccuggaggcccugcugaagcacaucuccuuc |
| | | gacaaguucgugcagaucucccugaaggcccugcggcggaucgugccccugauggagcagggcaagcgguacgacgaggccugcgccgaga |
| | | ucuacggcgaccacuacggcaagaagaacaccgaggagaagaucuaccugccccccauccccgccgacgagauccggaaccccguggugcu |
| | | gcgggcccugucccaggcccggaaggugaucaacggcguggugcggcgguacggcucccccgcccggauccacaucgagaccgcccgggag |
| | | gugggcaaguccuucaaggaccggaaggagaucgagaagcggcaggaggagaaccggaaggaccgggagaaggccgccgccaaguuccggg |
| | | aguacuuccccaacuucgugggcgagcccaaguccaaggacauccugaagcugcggcuguacgagcagcagcacggcaagugccuguacuc |
| | | cggcaaggagaucaaccuggugcggcugaacgagaagggcuacguggagaucgaccacgcccugcccuucucccggaccugggacgacucc |
| | | uucaacaacaaggugcuggugcugggcuccgagaaccagaacaagggcaaccagacccccuacgaguacuucaacggcaaggacaacuccc |
| | | gggaguggcaggaguucaaggcccggguggagaccucccgguucccccgguccaagaagcagcggauccugcugcagaaguucgacgagga |
| | | cggcuucaaggagugcaaccugaacgacacccgguacgugaaccgguuccugugccaguucguggccgaccacauccugcugaccggcaag |
| | | ggcaagcggcggguguucgccuccaacggccagaucaccaaccugcugcggggcuucuggggccugcggaagguggcggccgagaacgacc |
| | | ggcaccacgcccuggacgccguggugguggccugcuccaccguggccaugcagcagaagaucacccgguucgugcgguacaaggagaugaa |
| | | cgccuucgacggcaagaccaucgacaaggagaccggcaaggugcugcaccagaagacccacuucccccagcccugcggaguuuucgcccag |
| | | gaggugaugauccggguguucggcaagcccgacggcaagcccgaguucgaggaggccgacacccccgagaagcugcggacccugcuggccg |
| | | agaagcuguccucccggcccgaggccgugcacgaguacgugaccccccuguucgugucccgggcccccaaccggaagauguccggcgccca |
| | | caaggacacccugcgguccgccaagcgguucgugaagcacaacgagaagaucuccgugaagcggguguggcugaccgagaucaagcuggcc |
| | | gaccuggagaacauggugaacuacaagaacggccgggagaucgagcuguacgaggcccugaaggcccggcuggaggccuacggcggcaacg |
| | | ccaagcaggccuucgaccccaaggacaaccccuucuacaagaagggcggccagcuggugaaggccgugcggguggagaagacccaggaguc |
| | | cggcgugcugcugaacaagaagaacgccuacaccaucgccgacaacggcgacauggugcggguggacguguucugcaagguggacaagaag |
| | | ggcaagaaccaguacuucaucgugcccaucuacgccuggcagguggccgagaacauccugcccgacaucgacugcaagggcuaccggaucg |
| | | acgacuccuacaccuucugcuucucccugcacaaguacgaccugaucgccuuccagaaggacgagaaguccaagguggaguucgccuacua |
| | | caucaacugcgacuccuccaacggccgguucuaccuggccuggcacgacaagggcuccaaggagcagcaguuccggaucuccacccagaac |
| | | cuggugcugauccagaaguaccaggugaacgagcugggcaaggagauccggcccugccggcugaagaagcggccccccgugcgguag |
| 139 | Exemplary | AUGGCAGCAUUCAAGCCGAACUCGAUCAACUACAUCCUGGGACUGGACAUCGGAAUCGCAUCGGUCGGAUGGGCAAUGGUCGAAAUCGACG |
| | open | AAGAAGAAAACCCGAUCAGACUGAUCGACCUGGGAGUCAGAGUCUUCGAAAGAGCAGAAGUCCCGAAGACAGGAGACUCGCUGGCAAUGGC |
| | reading | AAGAAGACUGGCAAGAUCGGUCAGAAGACUGACAAGAAGAAGAGCACACAGACUGCUGAGAACAAGAAGACUGCUGAAGAGAGAAGGAGUC |
| | frame | CUGCAGGCAGCAAACUUCGACGAAAACGGACUGAUCAAGUCGCUGCCGAACACACCGUGGCAGCUGAGAGCAGCAGCACUGGACAGAAAGC |
| | for | UGACACCGCUGGAAUGGUCGGCAGUCCUGCUGCACCUGAUCAAGCACAGAGGAUACCUGUCGCAGAGAAAGAACGAAGGAGAAACAGCAGA |
| | Nme1Cas9 | CAAGGAACUGGGAGCACUGCUGAAGGGAGUCGCAGGAAACGCACACGCACUGCAGACAGGAGACUUCAGAACACCGGCAGAACUGGCACUG |
| | (mRNA | AACAAGUUCGAAAAGGAAUCGGGACACAUCAGAAACCAGAGAUCGGACUACUCGCACACAUUCUCGAGAAAGGACCUGCAGGCAGAACUGA |
| | AB | UCCUGCUGUUCGAAAAGCAGAAGGAAUUCGGAAACCCGCACGUCUCGGGAGGACUGAAGGAAGGAAUCGAAACACUGCUGAUGACACAGAG |
| | ORF) | ACCGGCACUGUCGGGAGACGCAGUCCAGAAGAUGCUGGGACACUGCACAUUCGAACCGGCAGAACCGAAGGCAGCAAAGAACACAUACACA |
| | | GCAGAAAGAUUCAUCUGGCUGACAAAGCUGAACAACCUGAGAAUCCUGGAACAGGGAUCGGAAAGACCGCUGACAGACACAGAAAGAGCAA |
| | | CACUGAUGGACGAACCGUACAGAAAGUCGAAGCUGACAUACGCACAGGCAAGAAAGCUGCUGGGACUGGAAGACACAGCAUUCUUCAAGGG |
| | | ACUGAGAUACGGAAAGGACAACGCAGAAGCAUCGACACUGAUGGAAAUGAAGGCAUACCACGCAAUCUCGAGAGCACUGGAAAAGGAAGGA |
| | | CUGAAGGACAAGAAGUCGCCGCUGAACCUGUCGCCGGAACUGCAGGACGAAAUCGGAACAGCAUUCUCGCUGUUCAAGACAGACGAAGACA |
| | | UCACAGGAAGACUGAAGGACAGAAUCCAGCCGGAAAUCCUGGAAGCACUGCUGAAGCACAUCUCGUUCGACAAGUUCGUCCAGAUCUCGCU |
| | | GAAGGCACUGAGAAGAAUCGUCCCGCUGAUGGAACAGGGAAAGAGAUACGACGAAGCAUGCGCAGAAAUCUACGGAGACCACUACGGAAAG |
| | | AAGAACACAGAAGAAAAGAUCUACCUGCCGCCGAUCCCGGCAGACGAAAUCAGAAACCCGGUCGUCCUGAGAGCACUGUCGCAGGCAAGAA |
| | | AGGUCAUCAACGGAGUCGUCAGAAGAUACGGAUCGCCGGCAAGAAUCCACAUCGAAACAGCAAGAGAAGUCGGAAAGUCGUUCAAGGACAG |
| | | AAAGGAAAUCGAAAAGAGACAGGAAGAAAACAGAAAGGACAGAGAAAAGGCAGCAGCAAAGUUCAGAGAAUACUUCCCGAACUUCGUCGGA |
| | | GAACCGAAGUCGAAGGACAUCCUGAAGCUGAGACUGUACGAACAGCAGCACGGAAAGUGCCUGUACUCGGGAAAGGAAAUCAACCUGGGAA |
| | | GACUGAACGAAAAGGGAUACGUCGAAAUCGACCACGCACUGCCGUUCUCGAGAACAUGGGACGACUCGUUCAACAACAAGGUCCUGGUCCU |
| | | GGGAUCGGAAAACCAGAACAAGGGAAACCAGACACCGUACGAAUACUUCAACGGAAAGGACAACUCGAGAGAAUGGCAGGAAUUCAAGGCA |
| | | AGAGUCGAAACAUCGAGAUUCCCGAGAUCGAAGAAGCAGAGAAUCCUGCUGCAGAAGUUCGACGAAGACGGAUUCAAGGAAAGAAACCUGA |
| | | ACGACACAAGAUACGUCAACAGAUUCCUGUGCCAGUUCGUCGCAGACAGAAUGAGACUGACAGGAAAGGGAAAGAAGAGAGUCUUCGCAUC |
| | | GAACGGACAGAUCACAAACCUGCUGAGAGGAUUCUGGGGACUGAGAAAGGUCAGAGCAGAAAACGACAGACACCACGCACUGGACGCAGUC |
| | | GUCGUCGCAUGCUCGACAGUCGCAAUGCAGCAGAAGAUCACAAGAUUCGUCAGAUACAAGGAAAUGAACGCAUUCGACGGAAAGACAAUCG |
| | | ACAAGGAAACAGGAGAAGUCCUGCACCAGAAGACACACUUCCCGCAGCCGUGGGAAUUCUUCGCACAGGAAGUCAUGAUCAGAGUCUUCGG |
| | | AAAGCCGGACGGAAAGCCGGAAUUCGAAGAAGCAGACACACUGGAAAAGCUGAGAACACUGCUGGCAGAAAAGCUGUCGUCGAGACCGGAA |
| | | GCAGUCCACGAAUACGUCACACCGCUGUUCGUCUCGAGAGCACCGAACAGAAAGAUGUCGGGACAGGGACACAUGGAAACAGUCAAGUCGG |
| | | CAAAGAGACUGGACGAAGGAGUCUCGGUCCUGAGAGUCCCGCUGACACAGCUGAAGCUGAAGGACCUGGAAAAGAUGGUCAACAGAGAAAG |
| | | AGAACCGAAGCUGUACGAAGCACUGAAGGCAAGACUGGAAGCACACAAGGACGACCCGGCAAAGGCAUUCGCAGAACCGUUCUACAAGUAC |
| | | GACAAGGCAGGAAACAGAACACAGCAGGUCAAGGCAGUCAGAGUCGAACAGGUCCAGAAGACAGGAGUCUGGGUCAGAAACCACAACGGAA |
| | | UCGCAGACAACGCAACAAUGGUCAGAGUAGACGUCUUCGAAAAGGGAGACAAGUACUACCUGGUCCCGAUCUACUCGUGGCAGGUCGCAAA |
| | | GGGAAUCCUGCCGGACAGAGCAGUCGUCCAGGGAAAGGACGAAGAAGACUGGCAGCUGAUCGACGACUCGUUCAACUUCAAGUUCUCGCUG |
| | | CACCCGAACGACCUGGUCGAAGUCAUCACAAAGAAGGCAAGAAUGUUCGGAUACUUCGCAUCGUGCCACAGAGGAACAGGAAACAUCAACA |
| | | UCAGAAUCCACGACCUGGACCACAAGAUCGGAAAGAACGGAAUCCUGGAAGGAAUCGGAGUCAAGACAGCACUGUCGUUCCAGAAGUACCA |
| | | GAUCGACGAACUGGGAAAGGAAAUCAGACCGUGCAGACUGAAGAAGAGACCGCCGGUCAGAUCCGGAAAGAGAACAGCAGACGGAUCGGAA |
| | | UUCGAAUCGCCGAAGAAGAAGAGAAAGGUCGAAUGA |
| 140 | WT Smu | AUGGAAAAAUUUCACUAUGUAUUGGGUUUGGAUUUGGGUAUCGCCUCUGUGGGGUGGGCUGCCAUUGAAAUUGACAAGGAAACCGAAACAU |
| | Cas9 | CAAUCGGUUUAUUGGAUUGCGGUGUCAGAACAUUUGAACGUGCAGAAGUACCCAAAACAGGCGAUUCUCUUGCCAAAGCUCGCCGUGAAGC |
| | coding | CAGAAGUACUCGCCGUUUAAUUCGCAGACGUUCGCAUCGCUUAUUACGUUUAAAACGUUUAUUGAAACGUGAAAUUUUCAGGCAGCCUGAA |
| | sequence | ACGUUUAAAGACUUACCAAUCAAUGCUUGGCAAUUGCGUGUUAAAGGCUUGGAUAGUCGGUUGAAUGAAUAUGAAUGGGCGGCCGUUUUAU |
| | | UGCAUUUGGUGAAGCAUCGCGGUUAUUUAUCGCAACGCAAAAGCGAAAUGAGCGAAACAGACAGCAAAUCUGAAAUGGGCAGAUUACUGGC |
| | | AGGUGUGGCGGAAAAUCACCAACUUUUACAACAAGAACAAUAUCGUACACCAGCCGAAUUAGCACUCAAAAAAUUUGUGAAACAUUUUCGC |
| | | AAUAAAGGUGGCGAUUAUGCACACACUUUCAACCGUUUGGAUUUGCAAGCCGAAUUGCAUUUAUUGUUUCAAAAACAACGUGAAUUAGGCA |
| | | AUCCAUUCACUUCACCAGAAUUGGAACGGCAAGUUGAUGAUUUGUUGAUGACGCAGCGCAGUGCUUUACAAGGUGAUGCGAUUUUGAAAAU |
| | | GUUGGGUCAUUGUGGGUUUGAACCUGAACAAUUCAAAGCAGCGAAAAACACAUUCAGUGCCGAACGUUUUAUUUGGUUGACAAAACUCAAU |
| | | AAUCUUCGCAUUCAAGACCAAGGCAAAGAACGUGCGUUAACUGCCGAUGAGCGUACCAAAUUGUUGGACGAGCCUUAUAAAAAAAGUAAAU |
| | | UGACUUACGCACAAGUUCGCAAAUUAUUAAGCUUGCCUCAAACUGCUAUUUUUAAAGGUUUGCGUUAUGAUUUGGAACAUGACAAAAAAGC |
| | | AGAAAACAGUACGUUGAUGGAAAUGAAAUCCUAUCACAACAUCCGCCAAACAUUGGAAAAAUCAGGUUUGAAAACAGAAUGGCAAAGUAUU |
| | | GCCACGCAGCCUGAAAUUUUAGAUGCAAUUGGCACGGCGUUUUCCAUUUAUAAAACCGAUGAAGAUAUUUCGCAUGAAUUAAAAACGUGCA |
| | | GGCUGCCUGAAAACGUAUUGAAUGAAUUACUGAAAAACAUCAAUUUUGAUGGAUUCAUUCAAUUAUCGUUGACUGCAUUACGCAAAAUUUU |
| | | GCCCUUGAUGGAACAAGGCUACCGUUAUGAUGAAGCGUGUACCCAAAUUUACGGUAAUCAUCAUUCAGGCAGCUUGCAACAAGAAUCAAAG |
| | | CAAUUUUUGCCACAUAUUCCGAUUGAUGAUGUCCGAAAUCCUGUGGUGUUCCGUACUUUGACCCAAGCAAGAAAAGUGGUGAAUGCGAUUA |
| | | UUCGUCGGUAUGGUUCGCCAGCUCGUGUGCAUAUUGAGAUGGCGCGUGAAUUGGGUAAGUCUAAAUCAGACCGUGACCGAAUUGAAAAACA |
| | | ACAACAAAAAAAUAAAAAAGAACGUGAAAACGCAGUCGCCAAAUUCAAAGAAGAUUUCCCUGAUUUUGUGGGCGAACCCAGAGGGAAAGAU |
| | | AUUUUGAAAAUGCGUUUGUAUGAACAACAACACGGCAAAUGUUUGUAUUCGGGUCAUGAUAUUGAUAUUAAUCGAUUGAAUGAAAAAGGUU |
| | | AUGUUGAAAUUGACCAUGCCCUGCCAUUUUCACGGACUUGGGAUGAUAGUCAAAAUAAUAAAGUUUUGGUACUUGGCAGCGAAAACCAAAA |
| | | UAAACGCAAUCAAACGCCUGAUGAAUAUUUGGACGGUGCAAACAAUAGCCAACGUUGGCUUGAAUUUCAAGCGCGUGUACAAACUUGUCAU |
| | | UUUUCUUACGGUAAAAAACAACGCAUUCAAUUAGCCAAAUUAGACGAUGAAACCGAAAAAGGAUUUUUAGAACGCAAUCUAAAUGAUACGC |
| | | GGUAUAUUGCUCGUUUUAUGUGUCAAUUUGUCCAAGAAAAUUUAUAUUUGACAGGUAAAGGAAAACGUCUUGUUUUUGCAUCAAACGGCGG |
| | | AAUGACCGCAACAUUGAGAAAUUUAUGGGGUUUGAGAAAAGUCCGUGAAGACAACGACCGCCAUCAUGCUCUUGAUGCGAUUGUGGUGGCG |
| | | UGUUCCACUGCUUCUAUGCAACAAAAAAUAACCAAAGCAUUUCAACGCCAUGAAAGCAUUGAAUAUGUGGAUACCGAAACGGGCGAAGUAA |
| | | AAUUUCGUAUUCCACAGCCUUGGGAUUUUUUCCGUCAGGAAGUGAUGAUUCGUGUGUUCAGCGACCAACCGUGUGAAGAUUUGGUAGAAAA |
| | | AUUGUCGGCUCGUCCCGAAGCUUUGCAUGACAACGUAACGCCUUUAUUUGUCUCGCGUGCACCAAAUCGCAAAAUGUCGGGGCAAGGGCAU |
| | | UUGGAAACCAUCAAAUCUGCAAAAAGGCUGUCUGAAGAAAACAGUAUGGUUAAAAAACCAUUAACCACAUUGAAAUUAAAAGAUAUUCCAG |
| | | AAAUCGUAGGCUACCCGAGUCGUGAACCUCAAUUGUAUGCCGCAUUGAAAACACGUUUAGAAACGCAUGAUGAUGACCCAAUUAAAGCCUU |
| | | UGCCAAACCCUUUUACAAACCCAAUAAAAAUGGUGAAUUGGGGGCGUUGGUUCGAUCGGUGCGUGUGAAAGGUGUACAAAAUACGGGUGUA |
| | | AUGGUUCAUGAUGGCAAAGGCAUUGCCGAUAAUGCCACAAUGGUUCGUGUUGAUGUCUAUACCAAAGCGGGCAAAAAUUACCUUGUUCCUG |
| | | UGUAUGUUUGGCAGGUGGCUCAAGGAAUUUUGCCAAAUCGGGCGGUUACUUCUGGCAAAAGUGAAGCAGAUUGGGAUUUAAUUGAUGAAAG |
| | | UUUUGAAUUUAAAUUUUCGCUGUCUCGUGGGGAUUUAGUGGAAAUGAUUAGCAAUAAAGGAAGAAUUUUUGGUUAUUACAAUGGGUUAGAU |
| | | CGUGCAAAUGGAAGUAUUGGGAUUCGUGAACAUGAUUUGGAAAAGUCCAAAGGAAAAGAUGGUGUUCAUCGUGUUGGCGUGAAAACCGCCA |
| | | CCGCAUUCAACAAAUACCACGUUGACCCACUUGGUAAAGAAAUUCAUCGGUGUUCAUCUGAACCACGCCCCACAUUAAAAAUCAAAUCCAA |
| | | GAAAUAA |
| 141 | human | ATGGAGAAGTTCCACTACGTGCTGGGACTGGATCTGGGAATCGCAAGCGTGGGATGGGCAGCAATCGAGATCGATAAGGAGACCGAGACAT |
| | codon- | CCATCGGCCTGCTGGACTGCGGCGTGAGGACCTTTGAGAGGGCAGAGGTGCCTAAGACAGGCGACAGCCTGGCAAAGGCAAGGAGAGAGGC |
| | optimized | AAGGTCTACAAGGCGCCTGATCCGGAGAAGGAGCCACAGGCTGCTGCGGCTGAAGAGACTGCTGAAGCGGGAGATCTTTAGACAGCCAGAG |
| | Smu | ACCTTCAAGGATCTGCCCATCAACGCATGGCAGCTGAGGGTGAAGGGACTGGACTCTCGGCTGAATGAGTACGAGTGGGCAGCCGTGCTGC |
| | coding | TGCACCTGGTGAAGCACAGGGGCTATCTGAGCCAGCGCAAGTCCGAGATGTCTGAGACCGACTCTAAGAGCGAGATGGGCAGGCTGCTGGC |
| | sequence | AGGAGTGGCCGAGAACCACCAGCTGCTGCAGCAGGAGCAGTACAGGACCCCAGCAGAGCTGGCCCTGAAGAAGTTTGTGAAGCACTTCCGC |
| | | AACAAGGGCGGCGATTATGCCCACACATTCAATAGGCTGGACCTGCAGGCAGAGCTGCACCTGCTGTTTCAGAAGCAGAGAGAGCTGGGCA |
| | | ACCCCTTCACCTCTCCTGAGCTGGAGCGCCAGGTGGACGATCTGCTGATGACACAGCGGAGCGCCCTGCAGGGCGATGCAATCCTGAAGAT |
| | | GCTGGGCCACTGTGGCTTTGAGCCTGAGCAGTTCAAGGCCGCCAAGAATACCTTTAGCGCCGAGAGATTCATCTGGCTGACAAAGCTGAAC |
| | | AATCTGAGGATCCAGGACCAGGGCAAGGAGAGAGCCCTGACCGCCGATGAGAGGACAAAGCTGCTGGACGAGCCTTACAAGAAGTCTAAGC |
| | | TGACCTATGCCCAGGTGAGGAAGCTGCTGAGCCTGCCTCAGACAGCCATCTTCAAGGGCCTGCGCTACGATCTGGAGCACGACAAGAAGGC |
| | | CGAGAACTCTACCCTGATGGAGATGAAGAGCTATCACAATATCCGGCAGACACTGGAGAAGTCCGGCCTGAAGACCGAGTGGCAGTCTATC |
| | | GCCACACAGCCAGAGATCCTGGACGCAATCGGAACCGCCTTTTCCATCTACAAGACAGATGAGGACATCTCTCACGAGCTGAAGACCTGCA |
| | | GACTGCCTGAGAACGTGCTGAATGAGCTGCTGAAGAACATCAATTTTGATGGCTTCATCCAGCTGAGCCTGACCGCCCTGCGCAAGATCCT |
| | | GCCACTGATGGAGCAGGGCTACCGGTATGACGAGGCCTGTACACAGATCTACGGCAACCACCACTCCGGCTCTCTGCAGCAGGAGTCCAAG |
| | | CAGTTTCTGCCTCACATCCCAATCGACGATGTGCGGAACCCAGTGGTGTTCAGAACCCTGACACAGGCCAGGAAGGTGGTGAATGCCATCA |
| | | TCCGCCGGTATGGATCTCCAGCAAGGGTGCACATCGAGATGGCAAGGGAGCTGGGCAAGAGCAAGTCCGATAGAGACAGGATCGAGAAGCA |
| | | GCAGCAGAAGAACAAGAAGGAGAGGGAGAATGCCGTGGCCAAGTTCAAGGAGGATTTTCCAGACTTCGTGGGAGAGCCTAGGGGCAAGGAT |
| | | ATCCTGAAGATGCGGCTGTACGAGCAGCAGCACGGCAAGTGCCTGTATTCCGGCCACGATATCGACATCAACCGGCTGAATGAGAAGGGCT |
| | | ACGTGGAGATCGACCACGCCCTGCCTTTTAGCAGAACCTGGGACGATTCCCAGAACAATAAGGTGCTGGTGCTGGGCAGCGAGAACCAGAA |
| | | TAAGCGCAATCAGACACCAGATGAGTACCTGGACGGCGCCAACAATTCCCAGAGATGGCTGGAGTTTCAGGCCAGGGTGCAGACCTGCCAC |
| | | TTCTCTTATGGCAAGAAGCAGAGGATCCAGCTGGCCAAGCTGGACGATGAGACCGAGAAGGGCTTCCTGGAGCGCAACCTGAATGATACAA |
| | | GGTACATCGCCCGGTTCATGTGCCAGTTCGTGCAGGAGAACCTGTATCTGACCGGCAAGGGCAAGCGCCTGGTGTTTGCCTCCAACGGCGG |
| | | CATGACCGCCACACTGCGGAATCTGTGGGGCCTGAGGAAGGTGCGCGAGGATAATGACAGACACCACGCACTGGACGCAATCGTGGTGGCA |
| | | TGCAGCACCGCATCCATGCAGCAGAAGATCACAAAGGCCTTTCAGCGGCACGAGAGCATCGAGTATGTGGATACCGAGACAGGCGAGGTGA |
| | | AGTTCAGAATCCCCCAGCCTTGGGACTTCTTTCGCCAGGAAGTGATGATCCGGGTGTTTTCCGATCAGCCATGTGAGGACCTGGTGGAGAA |
| | | GCTGTCTGCCAGGCCAGAGGCCCTGCACGATAACGTGACCCCTCTGTTCGTGAGCAGGGCACCAAATAGAAAGATGTCCGGCCAGGGCCAC |
| | | CTGGAGACAATCAAGTCCGCCAAGCGCCTGTCCGAGGAGAACTCTATGGTGAAGAAGCCCCTGACCACACTGAAGCTGAAGGACATCCCTG |
| | | AGATCGTGGGCTACCCATCTAGAGAGCCCCAGCTGTATGCCGCCCTGAAGACCAGGCTGGAGACACACGACGATGACCCAATCAAGGCCTT |
| | | TGCCAAGCCTTTCTACAAGCCAAACAAGAATGGAGAGCTGGGCGCCCTGGTGCGGTCCGTGAGAGTGAAGGGCGTGCAGAACACAGGCGTG |
| | | ATGGTGCACGATGGCAAGGGCATCGCCGACAATGCCACAATGGTGCGGGTGGACGTGTATACAAAGGCCGGCAAGAACTACCTGGTGCCCG |
| | | TGTACGTGTGGCAGGTGGCACAGGGAATCCTGCCAAATAGAGCCGTGACCTCTGGCAAGAGCGAGGCCGATTGGGACCTGATCGATGAGAG |
| | | CTTCGAGTTTAAGTTCTCTCTGAGCAGAGGCGACCTGGTGGAGATGATCTCCAACAAGGGCAGGATCTTCGGCTACTATAACGGCCTGGAT |
| | | AGAGCCAATGGCAGCATCGGCATCAGGGAGCACGATCTGGAGAAGTCCAAGGGCAAGGACGGAGTGCACAGGGTGGGAGTGAAGACCGCAA |
| | | CAGCCTTTAATAAGTACCACGTGGACCCCCTGGGCAAGGAGATCCACAGATGTAGCTCCGAGCCAAGGCCCACCCTGAAGATCAAGAGCAA |
| | | GAAG |
| 142 | WT Smu | MEKFHYVLGLDLGIASVGWAAIEIDKETETSIGLLDCGVRTFERAEVPKTGDSLAKARREARSTRRLIRRRSHRLLRLKRLLKREIFRQPE |
| | Cas9 | TFKDLPINAWQLRVKGLDSRLNEYEWAAVLLHLVKHRGYLSQRKSEMSETDSKSEMGRLLAGVAENHQLLQQEQYRTPAELALKKFVKHFR |
| | amino | NKGGDYAHTFNRLDLQAELHLLFQKQRELGNPFTSPELERQVDDLLMTQRSALQGDAILKMLGHCGFEPEQFKAAKNTFSAERFIWLTKLN |
| | acid | NLRIQDQGKERALTADERTKLLDEPYKKSKLTYAQVRKLLSLPQTAIFKGLRYDLEHDKKAENSTLMEMKSYHNIRQTLEKSGLKTEWQSI |
| | sequen | ATQPEILDAIGTAFSIYKTDEDISHELKTCRLPENVLNELLKNINFDGFIQLSLTALRKILPLMEQGYRYDEACTQIYGNHHSGSLQQESK |
| | ce | QFLPHIPIDDVRNPVVFRTLTQARKVVNAIIRRYGSPARVHIEMARELGKSKSDRDRIEKQQQKNKKERENAVAKFKEDFPDFVGEPRGKD |
| | | ILKMRLYEQQHGKCLYSGHDIDINRLNEKGYVEIDHALPFSRTWDDSQNNKVLVLGSENQNKRNQTPDEYLDGANNSQRWLEFQARVQTCH |
| | | FSYGKKQRIQLAKLDDETEKGFLERNLNDTRYIARFMCQFVQENLYLTGKGKRLVFASNGGMTATLRNLWGLRKVREDNDRHHALDAIVVA |
| | | CSTASMQQKITKAFQRHESIEYVDTETGEVKFRIPQPWDFFRQEVMIRVFSDQPCEDLVEKLSARPEALHDNVTPLFVSRAPNRKMSGQGH |
| | | LETIKSAKRLSEENSMVKKPLTTLKLKDIPEIVGYPSREPQLYAALKTRLETHDDDPIKAFAKPFYKPNKNGELGALVRSVRVKGVQNTGV |
| | | MVHDGKGIADNATMVRVDVYTKAGKNYLVPVYVWQVAQGILPNRAVTSGKSEADWDLIDESFEFKFSLSRGDLVEMISNKGRIFGYYNGLD |
| | | RANGSIGIREHDLEKSKGKDGVHRVGVKTATAFNKYHVDPLGKEIHRCSSEPRPTLKIKSKK |
| 143 | WT Cje | ATGGCAAGAATTTTGGCATTTGATATAGGAATAAGCTCTATTGGCTGGGCATTTAGTGAAAATGATGAGT |
| | Cas9 | TAAAAGATTGTGGAGTTAGGATTTTTACAAAGGTTGAAAATCCCAAAACAGGAGAATCCTTAGCCTTGCC |
| | coding | AAGACGCCTTGCAAGAAGTGCTAGAAAACGCCTTGCTAGACGCAAAGCAAGGCTAAATCACTTAAAACAT |
| | sequence | TTAATCGCAAATGAATTTAAGTTAAATTACGAAGATTATCAAAGCTTTGATGAAAGTTTAGCCAAGGCTT |
| | | ATAAAGGATCTTTAATTAGCCCTTATGAGTTAAGATTTAGAGCTTTAAACGAGCTTTTAAGTAAGCAAGA |
| | | TTTTGCAAGAGTGATTTTACATATTGCAAAAAGGCGTGGCTATGATGATATTAAAAATAGTGATGATAAA |
| | | GAAAAAGGTGCGATTTTAAAAGCTATCAAGCAAAATGAAGAAAAACTAGCAAATTATCAAAGTGTGGGCG |
| | | AGTATCTTTATAAAGAGTATTTTCAAAAATTCAAAGAAAATTCCAAAGAATTTACAAATGTTAGAAATAA |
| | | AAAAGAAAGTTATGAACGCTGCATAGCGCAAAGTTTTTTAAAAGATGAATTAAAGCTTATTTTTAAAAAA |
| | | CAAAGAGAATTTGGCTTTTCTTTTTCAAAAAAATTTGAAGAAGAGGTGCTTAGTGTAGCATTTTATAAAA |
| | | GGGCTTTAAAAGATTTTTCTCATTTGGTGGGAAATTGTAGTTTTTTTACCGATGAAAAAAGAGCGCCAAA |
| | | AAATTCGCCCTTAGCTTTTATGTTTGTAGCACTTACAAGAATTATCAATCTTTTAAACAATCTTAAAAAT |
| | | ACTGAAGGAATTTTATATACAAAAGATGATTTAAACGCCCTTTTAAATGAAGTTTTAAAAAACGGCACTC |
| | | TAACTTATAAACAAACAAAAAAGCTTTTGGGCTTAAGCGATGATTATGAATTTAAAGGAGAAAAAGGAAC |
| | | TTATTTTATTGAGTTTAAAAAATACAAAGAATTTATAAAAGCCCTAGGTGAGCATAATTTAAGCCAAGAT |
| | | GATTTAAATGAAATAGCAAAAGATATTACTCTTATAAAAGATGAGATTAAACTTAAAAAGGCTTTAGCAA |
| | | AATATGATTTAAATCAAAATCAAATTGATAGTTTAAGCAAATTGGAATTTAAAGACCATTTAAATATAAG |
| | | TTTTAAAGCCTTAAAACTTGTCACTCCTTTAATGCTTGAAGGTAAAAAATACGATGAAGCTTGCAATGAG |
| | | CTTAATTTAAAAGTAGCAATCAATGAAGATAAAAAAGACTTTTTACCCGCATTTAATGAAACTTATTACA |
| | | AAGATGAAGTAACTAATCCTGTGGTTTTAAGGGCGATTAAAGAATATAGAAAGGTCTTAAATGCCTTACT |
| | | TAAAAAATACGGTAAAGTGCATAAAATAAACATTGAATTAGCAAGAGAAGTAGGTAAAAATCACAGCCAA |
| | | AGAGCTAAAATAGAAAAAGAGCAAAACGAAAACTACAAGGCCAAAAAAGACGCCGAACTTGAATGTGAAA |
| | | AACTAGGCTTAAAAATAAATAGCAAAAATATTTTAAAATTAAGATTATTTAAAGAACAAAAAGAATTTTG |
| | | TGCTTATAGTGGAGAAAAGATTAAAATTAGTGATTTGCAAGATGAAAAAATGCTTGAAATTGATCACATT |
| | | TACCCTTATTCTAGAAGTTTTGATGATTCTTATATGAATAAAGTTTTAGTTTTTACCAAGCAAAATCAAG |
| | | AAAAACTTAATCAAACCCCCTTTGAAGCCTTTGGCAATGATAGTGCAAAATGGCAGAAAATTGAAGTTTT |
| | | AGCAAAAAATTTGCCTACTAAAAAGCAAAAAAGAATTTTAGATAAAAATTACAAAGACAAAGAGCAAAAA |
| | | AACTTTAAAGATAGAAATCTAAACGATACTCGCTACATAGCAAGGCTTGTTTTAAACTACACTAAAGATT |
| | | ATTTAGATTTTTTACCTTTAAGCGATGATGAAAATACAAAATTAAACGATACACAAAAAGGCTCAAAAGT |
| | | GCATGTAGAAGCAAAAAGCGGTATGCTTACATCGGCTTTAAGACACACTTGGGGCTTTAGCGCTAAGGAT |
| | | AGAAATAACCATCTTCATCACGCCATCGATGCAGTAATTATTGCTTATGCAAACAACTCTATCGTAAAGG |
| | | CTTTTTCAGATTTTAAAAAAGAGCAAGAAAGCAACAGTGCAGAACTTTATGCAAAAAAAATTAGTGAATT |
| | | AGATTATAAAAACAAAAGGAAGTTTTTTGAGCCTTTTAGTGGTTTTAGGCAAAAGGTTTTAGATAAAATT |
| | | GATGAAATTTTTGTCTCAAAACCTGAGAGAAAAAAGCCAAGTGGAGCTTTACATGAAGAAACATTTAGAA |
| | | AAGAAGAAGAATTCTATCAATCATACGGCGGAAAAGAAGGAGTTTTAAAAGCCCTAGAGCTTGGTAAAAT |
| | | CCGCAAAGTAAATGGTAAAATAGTTAAAAATGGCGATATGTTTAGAGTGGATATTTTTAAACACAAAAAG |
| | | ACAAATAAATTTTATGCTGTGCCAATTTATACTATGGATTTTGCTTTAAAAGTATTACCAAATAAAGCTG |
| | | TAGCACGAAGTAAAAAAGGTGAGATAAAAGACTGGATTTTAATGGATGAAAATTATGAGTTTTGCTTTTC |
| | | TTTATATAAAGATAGTTTGATTTTAATTCAAACTAAAGATATGCAAGAGCCAGAATTTGTTTATTATAAT |
| | | GCTTTTACAAGCTCTACTGTTAGTTTGATTGTTTCAAAACACGATAATAAATTTGAAACGCTAAGCAAAA |
| | | ATCAAAAAATATTGTTTAAAAATGCAAATGAAAAAGAAGTAATCGCAAAAAGCATAGGAATTCAAAATCT |
| | | AAAAGTATTTGAAAAATACATAGTTTCAGCACTTGGGGAAGTTACAAAGGCTGAGTTTAGACAAAGAGAA |
| | | GATTTTAAAAAATGA |
| 144 | WT Cje | MARILAFDIGISSIGWAFSENDELKDCGVRIFTKVENPKTGESLALPRRLARSAR |
| | Cas9 | KRLARRKARLNHLKHLIANEFKLNYEDYQSFDESLAKAYKGSLISPYELRFRAL |
| | amino | NELLSKQDFARVILHIAKRRGYDDIKNSDDKEKGAILKAIKQNEEKLANYQSVG |
| | acid | EYLYKEYFQKFKENSKEFTNVRNKKESYERCIAQSFLKDELKLIFKKQREFGFSF |
| | sequence | SKKFEEEVLSVAFYKRALKDFSHLVGNCSFFTDEKRAPKNSPLAFMFVALTRIIN |
| | | LLNNLKNTEGILYTKDDLNALLNEVLKNGTLTYKQTKKLLGLSDDYEFKGEKG |
| | | TYFIEFKKYKEFIKALGEHNLSQDDLNEIAKDITLIKDEIKLKKALAKYDLNQNQ |
| | | IDSLSKLEFKDHLNISFKALKLVTPLMLEGKKYDEACNELNLKVAINEDKKDFL |
| | | PAFNETYYKDEVTNPVVLRAIKEYRKVLNALLKKYGKVHKINIELAREVGKNH |
| | | SQRAKIEKEQNENYKAKKDAELECEKLGLKINSKNILKLRLFKEQKEFCAYSGE |
| | | KIKISDLQDEKMLEIDHIYPYSRSFDDSYMNKVLVFTKQNQEKLNQTPFEAFGN |
| | | DSAKWQKIEVLAKNLPTKKQKRILDKNYKDKEQKNFKDRNLNDTRYIARLVL |
| | | NYTKDYLDFLPLSDDENTKLNDTQKGSKVHVEAKSGMLTSALRHTWGFSAKD |
| | | RNNHLHHAIDAVIIAYANNSIVKAFSDFKKEQESNSAELYAKKISELDYKNKRK |
| | | FFEPFSGFRQKVLDKIDEIFVSKPERKKPSGALHEETFRKEEEFYQSYGGKEGVL |
| | | KALELGKIRKVNGKIVKNGDMFRVDIFKHKKTNKFYAVPIYTMDFALKVLPNK |
| | | AVARSKKGEIKDWILMDENYEFCFSLYKDSLILIQTKDMQEPEFVYYNAFTSST |
| | | VSLIVSKHDNKFETLSKNQKILFKNANEKEVIAKSIGIQNLKVFEKYIVSALGEVT |
| | | KAEFRQREDFKK |
| 145- | | NQT USED |
| 149 | | |
| 150 | Exemplary | GAPESATESGGTSTESEGSAGTSTESEGSAGSAGSTSTEEGTSTESEGSAGTSTESEGSAGTSESATESGGTSTESEGSSS |
| | protein | |
| | linker | |
| | (81 aa) | |
| 151 | Exemplary | GAPESATESGGTSTESEGSAGTSTESEGSAGSAGSTSTEEGTSTESEGSAGTSTESEGSAGTSESATGSSS |
| | protein | |
| | linker | |
| | (71 aa) | |
| 152 | Exemplary | GAPESATESGGTSTESEGSAGTSTESEGSAGSAGSTSTEEGTSTESEGSAGTSTESEGSSS |
| | protein | |
| | linker | |
| | (61 aa) | |
| 153 | Exemplary | GAPESATESGGTSTESEGSAGTSTESEGSAGSAGSTSTEEGTSTESEGSSS |
| | protein | |
| | linker | |
| | (51 aa) | |
| 154 | Exemplary | GAPESATESGGTSTESEGSAGTSTESEGSAGSAGSTSGSSS |
| | protein | |
| | linker | |
| | (41 aa) | |
| 155 | Exemplary | GAPESATESGGTSTESEGSAGTSTESEGSSS |
| | protein | |
| | linker | |
| | (31 aa) | |
| 156 | Exemplary | GAPESATESGGTSTESEGSSS |
| | protein | |
| | linker | |
| | (21 aa) | |
| 157 | Exemplary | GAPESATGSSS |
| | protein | |
| | linker | |
| | (11 aa) | |
| 158 | Exemplary | GAPESATESGGTSTESEGSAGTSTESEGSAGSAGSTSGGSGGGSGGSGSAGTSTESEGSAGTSESATESGGTSTESEGSSS |
| | protein | |
| | linker | |
| | (81 aa) | |

TABLE 38
Additional Sequences

| SEQ | | |
|---|---|---|
| ID | | |
| NO | Description | Sequence |
| 200 | Exemplary Spy | mC*mC*mA*GUCCAGCGAGGCAAAGGGUUUUAGAmGmCmUmAmGmAmAmAmUmAmGmCAAGUUAAAAUAAGGCUAGUCCG |
| | guide; G000267 | UUAUCAmAmCmUmUmGmAmAmAmAmAmGmUmGmGmCmAmCmCmGmAmGmUmCmGmGmUmGmCmU*mU*mU*mU |
| 201 | Exemplary Spy | mA*mC*mA*CAAAUACCAGUCCAGCGGUUUUAGAmGmCmUmAmGmAmAmAmUmAmGmCAAGUUAAAAUAAGGCUAGUCCG |
| | guide; G000502 | UUAUCAmAmCmUmUmGmAmAmAmAmAmGmUmGmGmCmAmCmCmGmAmGmUmCmGmGmUmGmCmU*mU*mU*mU |
| 202 | Exemplary Spy | mC*mC*mC*AGAGGCAAAGGGCUCCCGUUUUAGAmGmCmUmAmGmAmAmAmUmAmGmCAAGUUAAAAUAAGGCUAGUCCG |
| | guide; G000603 | UUAUCAmAmCmUmUmGmAmAmAmAmAmGmUmGmGmCmAmCmCmGmAmGmUmCmGmGmUmGmCmU*mU*mU*mU |
| 203 | Exemplary Spy | mA*mG*mU*GUUCAAAAAGACCUCUGGUUUUAGAmGmCmUmAmGmAmAmAmUmAmGmCAAGUUAAAAUAAGGCUAGUCCG |
| | guide; G000606 | UUAUCAmAmCmUmUmGmAmAmAmAmAmGmUmGmGmCmAmCmCmGmAmGmUmCmGmGmUmGmCmU*mU*mU*mU |
| 204 | Exemplary Spy | mC*mU*mC*CCUGAUGGAGAUGACAGGUUUUAGAmGmCmUmAmGmAmAmAmUmAmGmCAAGUUAAAAUAAGGCUAGUCCG |
| | guide; G000665 | UUAUCAmAmCmUmUmGmAmAmAmAmAmGmUmGmGmCmAmCmCmGmAmGmUmCmGmGmUmGmCmU*mU*mU*mU |
| 205 | Exemplary Spy | mC*mU*mU*UCUACAAGCUUACCCAGGUUUUAGAmGmCmUmAmGmAmAmAmUmAmGmCAAGUUAAAAUAAGGCUAGUCCG |
| | guide; G009577 | UUAUCAmAmCmUmUmGmAmAmAmAmAmGmUmGmGmCmAmCmCmGmAmGmUmCmGmGmUmGmCmU*mU*mU*mU |
| 206 | Exemplary Spy | CAGdTGCGGdTdTCCACACAdTACGdTdTdTdTAGAGCdTAGAAAdTAGCAAGdTdTAAAAdTAAGGCdTAGdTCCGdTd |
| | guide; G012684 | TAdTCAACdTdTGAAAAAGdTGGCACCGAGdTCGGdTGCdTdTdTdTdTdTdTGGdTACCGGdTACC |
| 207 | Exemplary Spy | AAGCCdTGCAdTGAGCdTCAGdTGGdTdTdTdTAGAGCdTAGAAAdTAGCAAGdTdTAAAAdTAAGGCdTAGdTCCGdTd |
| | guide; G012685 | TAdTCAACdTdTGAAAAAGdTGGCACCGAGdTCGGdTGCdTdTdTdTdTdTdTGGdTACCGGdTACC |
| 208 | Exemplary Spy | AdTGGGdTCACAGACGGGdTCCAGdTdTdTdTAGAGCdTAGAAAdTAGCAAGdTdTAAAAdTAAGGCdTAGdTCCGdTdT |
| | guide; G012686 | AdTCAACdTdTGAAAAAGdTGGCACCGAGdTCGGdTGCdTdTdTdTdTdTdTGGdTACCGGdTACC |
| 209 | Exemplary Spy | CdTCAAAGAGACCCAdTGAGAdTGdTdTdTdTAGAGCdTAGAAAdTAGCAAGdTdTAAAAdTAAGGCdTAGdTCCGdTdT |
| | guide; G012687 | AdTCAACdTdTGAAAAAGdTGGCACCGAGdTCGGdTGCdTdTdTdTdTdTdTGGdTACCGGdTACC |
| 210 | Exemplary Spy | AdTCdTCGdTAdTdTdTCAdTGdTCdTCCGdTdTdTdTAGAGCdTAGAAAdTAGCAAGdTdTAAAAdTAAGGCdTAGdTC |
| | guide; G012688 | CGdTdTAdTCAACdTdTGAAAAAGdTGGCACCGAGdTCGGdTGCdTdTdTdTdTdTdTGGdTACCGGdTACC |
| 211 | Exemplary Spy | CACCdTdTGGCdTGdTAGdTCAdTCdTGdTdTdTdTAGAGCdTAGAAAdTAGCAAGdTdTAAAAdTAAGGCdTAGdTCCG |
| | guide; G012746 | dTdTAdTCAACdTdTGAAAAAGdTGGCACCGAGdTCGGdTGCdTdTdTdTdTdTdTGGdTACCGGdTACC |
| 212 | Exemplary Spy | mA*mU*mA*CCAGUCCAGCGAGGCAAGUUUUAGAmGmCmUmAmGmAmAmAmUmAmGmCAAGUUAAAAUAAGGCUAGUCCG |
| | guide; G014304 | UUAUCAmAmCmUmUmGmAmAmAmAmAmGmUmGmGmCmAmCmCmGmAmGmUmCmGmGmUmGmCmU*mU*mU*mU |
| 213 | Exemplary Spy | mC*mC*mU*CCUUUGCCUCGCUGGACGUUUUAGAmGmCmUmAmGmAmAmAmUmAmGmCAAGUUAAAAUAAGGCUAGUCCG |
| | guide; G014317 | UUAUCAmAmCmUmUmGmAmAmAmAmAmGmUmGmGmCmAmCmCmGmAmGmUmCmGmGmUmGmCmU*mU*mU*mU |
| 214 | Exemplary Spy | mC*mU*mG*GUAUUUGUGUCUGAAGCGUUUUAGAmGmCmUmAmGmAmAmAmUmAmGmCAAGUUAAAAUAAGGCUAGUCCG |
| | guide; G014322 | UUAUCAmAmCmUmUmGmAmAmAmAmAmGmUmGmGmCmAmCmCmGmAmGmUmCmGmGmUmGmCmU*mU*mU*mU |
| 215 | Exemplary Spy | mG*mC*mA*AAGGAGGAAGAGUCGAAGUUUUAGAmGmCmUmAmGmAmAmAmUmAmGmCAAGUUAAAAUAAGGCUAGUCCG |
| | guide; G014323 | UUAUCAmAmCmUmUmGmAmAmAmAmAmGmUmGmGmCmAmCmCmGmAmGmUmCmGmGmUmGmCmU*U*mU*mU |
| 216 | Exemplary Spy | mC*mU*mC*UUCCUCCUUUGCCUCGCGUUUUAGAmGmCmUmAmGmAmAmAmUmAmGmCAAGUUAAAAUAAGGCUAGUCCG |
| | guide; G014325 | UUAUCAmAmCmUmUmGmAmAmAmAmAmGmUmGmGmCmAmCmCmGmAmGmUmCmGmGmUmGmCmU*mU*mU*mU |
| 217 | Exemplary Spy | mG*mG*mC*AAAGGAGGAAGAGUCGAGUUUUAGAmGmCmUmAmGmAmAmAmUmAmGmCAAGUUAAAAUAAGGCUAGUCCG |
| | guide; G014326 | UUAUCAmAmCmUmUmGmAmAmAmAmAmGmUmGmGmCmAmCmCmGmAmGmUmCmGmGmUmGmCmU*mU*mU*mU |
| 218 | Exemplary Spy | mU*mU*mU*GACCAUCAGAGGACAUUGUUUUAGAmGmCmUmAmGmAmAmAmUmAmGmCAAGUUAAAAUAAGGCUAGUCCG |
| | guide; G016128 | UUAUCAmAmCmUmUmGmAmAmAmAmAmGmUmGmGmCmAmCmCmGmAmGmUmCmGmGmUmGmCmU*mU*mU*mU |
| 219 | Exemplary Spy | mG*mG*mG*CUCCCAGGAUCCCUCAGGUUUUAGAmGmCmUmAmGmAmAmAmUmAmGmCAAGUUAAAAUAAGGCUAGUCCG |
| | guide; G016133 | UUAUCAmAmCmUmUmGmAmAmAmAmAmGmUmGmGmCmAmCmCmGmAmGmUmCmGmGmUmGmCmU*mU*mU*mU |
| 220 | Exemplary Spy | mA*mA*mA*GACCUCUGAGGGAUCCUGUUUUAGAmGmCmUmAmGmAmAmAmUmAmGmCAAGUUAAAAUAAGGCUAGUCCG |
| | guide; G016138 | UUAUCAmAmCmUmUmGmAmAmAmAmAmGmUmGmGmCmAmCmCmGmAmGmUmCmGmGmUmGmCmU*mU*mU*mU |
| 221 | Exemplary Spy | mU*mC*mC*UGGGAGCCCUUUGCCUCGUUUUAGAmGmCmUmAmGmAmAmAmUmAmGmCAAGUUAAAAUAAGGCUAGUCCG |
| | guide; G016139 | UUAUCAmAmCmUmUmGmAmAmAmAmAmGmUmGmGmCmAmCmCmGmAmGmUmCmGmGmUmGmCmU*mU*mU*mU |
| 222 | Exemplary Spy | mA*mA*mA*AGACCUCUGAGGGAUCCGUUUUAGAmGmCmUmAmGmAmAmAmUmAmGmCAAGUUAAAAUAAGGCUAGUCCG |
| | guide; G016145 | UUAUCAmAmCmUmUmGmAmAmAmAmAmGmUmGmGmCmAmCmCmGmAmGmUmCmGmGmUmGmCmU*mU*mU*mU |
| 223 | Exemplary Spy | mG*mU*mG*UUCAAAAAGACCUCUGAGUUUUAGAmGmCmUmAmGmAmAmAmUmAmGmCAAGUUAAAAUAAGGCUAGUCCG |
| | guide; G016146 | UUAUCAmAmCmUmUmGmAmAmAmAmAmGmUmGmGmCmAmCmCmGmAmGmUmCmGmGmUmGmCmU*mU*mU*mU |
| 224 | Exemplary Spy | mA*mG*mA*AUCCAAAUGUCCUCUGAGUUUUAGAmGmCmUmAmGmAmAmAmUmAmGmCAAGUUAAAAUAAGGCUAGUCCG |
| | guide; G016148 | UUAUCAmAmCmUmUmGmAmAmAmAmAmGmUmGmGmCmAmCmCmGmAmGmUmCmGmGmUmGmCmU*mU*mU*mU |
| 225 | Exemplary Spy | mA*mC*mA*AGCUUACCCAGAGGCAAGUUUUAGAmGmCmUmAmGmAmAmAmUmAmGmCAAGUUAAAAUAAGGCUAGUCCG |
| | guide; G016157 | UUAUCAmAmCmUmUmGmAmAmAmAmAmGmUmGmGmCmAmCmCmGmAmGmUmCmGmGmUmGmCmU*mU*mU*mU |
| 226 | Exemplary Spy | mC*mA*mA*GCUUACCCAGAGGCAAAGUUUUAGAmGmCmUmAmGmAmAmAmUmAmGmCAAGUUAAAAUAAGGCUAGUCCG |
| | guide; G016163 | UUAUCAmAmCmUmUmGmAmAmAmAmAmGmUmGmGmCmAmCmCmGmAmGmUmCmGmGmUmGmCmU*mU*mU*mU |
| 227 | Exemplary Spy | mC*mU*mC*CUCUGUGCCCAGGGUGCGUUUUAGAmGmCmUmAmGmAmAmAmUmAmGmCAAGUUAAAAUAAGGCUAGUCCG |
| | guide; G016164 | UUAUCAmAmCmUmUmGmAmAmAmAmAmGmUmGmGmCmAmCmCmGmAmGmUmCmGmGmUmGmCmU*mU*mU*mU |
| 228 | Exemplary Spy | mC*mU*mA*CUGUGCCCCACCGGCGCGUUUUAGAGCUAGAAAUAGCAAGUUAAAAUAAGGCUAGUCCGUUAUCAAAAUGG |
| | guide; G017875 | CACCGAGUCGGU*mG*mC*mU |
| 229 | Exemplary Spy | mG*mC*mU*GGUGCCCAGGACGAGGAGUUUUAGAGCUAGAAAUAGCAAGUUAAAAUAAGGCUAGUCCGUUAUCAAAAUGG |
| | guide; G017876 | CACCGAGUCGGU*mG*mC*mU |
| 230 | Exemplary Spy | mC*mA*mC*CUUCCGCCGUUGCUCCAGUUUUAGAGCUAGAAAUAGCAAGUUAAAAUAAGGCUAGUCCGUUAUCAAAAUGG |
| | guide; G017877 | CACCGAGUCGGU*mG*mC*mU |
| 231 | Exemplary Spy | mC*mC*mG*CCGUUGCUCCAAGGUAUGUUUUAGAGCUAGAAAUAGCAAGUUAAAAUAAGGCUAGUCCGUUAUCAAAAUGG |
| | guide; G017879 | CACCGAGUCGGU*mG*mC*mU |
| 232 | Exemplary Spy | mA*mA*mG*GUAUGGGUGCCAGGCAAGUUUUAGAGCUAGAAAUAGCAAGUUAAAAUAAGGCUAGUCCGUUAUCAAAAUGG |
| | guide; G017881 | CACCGAGUCGGU*mG*mC*mU |
| 233 | Exemplary Spy | mG*mU*mU*GCCUGGCACCCAUACCUGUUUUAGAGCUAGAAAUAGCAAGUUAAAAUAAGGCUAGUCCGUUAUCAAAAUGG |
| | guide; G017892 | CACCGAGUCGGU*mG*mC*mU |
| 234 | Exemplary Spy | mG*mC*mA*CCCAUACCUUGGAGCAAGUUUUAGAGCUAGAAAUAGCAAGUUAAAAUAAGGCUAGUCCGUUAUCAAAAUGG |
| | guide; G017893 | CACCGAGUCGGU*mG*mC*mU |
| 235 | Exemplary Spy | mA*mU*mA*AUCUCCAUCCUCGUCCUGUUUUAGAGCUAGAAAUAGCAAGUUAAAAUAAGGCUAGUCCGUUAUCAAAAUGG |
| | guide; G017896 | CACCGAGUCGGU*mG*mC*mU |
| 236 | Exemplary Spy | mC*mC*mG*CCGUUGCUCCAAGGUAUGUUUUAGAmGmCmUmAmGmAmAmAmUmAmGmCAAGUUAAAAUAAGGCUAGUCCG |
| | guide; G018723 | UUAUCAmAmCmUmUmGmAmAmAmAmAmGmUmGmGmCmAmCmCmGmAmGmUmCmGmGmUmGmCmU*mU*mU*mU |
| 237 | Exemplary Spy | mG*mC*mA*CCCAUACCUUGGAGCAAGUUUUAGAmGmCmUmAmGmAmAmAmUmAmGmCAAGUUAAAAUAAGGCUAGUCCG |
| | guide; G018726 | UUAUCAmAmCmUmUmGmAmAmAmAmAmGmUmGmGmCmAmCmCmGmAmGmUmCmGmGmUmGmCmU*mU*mU*mU |
| 238 | Exemplary Nme | GGGCGCUUCGCCAGCCAUUCCUGCGUUGUAGCUCCCUUUCUCAUUUCGGAAACGAAAUGAGAACCGUUGCUACAAUAAGG |
| | guide; plCR012468 | CCGUCUGAAAAGAUGUGCCGCAACGCUCUGCCCCUUAAAGCUUCUGCUUUAAGGGGCAUCGUUUAU |
| 239 | Exemplary Nme | GCCGGGGCGGTCCCGGTCCAAGCGGUUGUAGCUCCCUUUCUCAUUUCGGAAACGAAAUGAGAACCGUUGCUACAAUAAGG |
| | guide; plCR012469 | CCGUCUGAAAAGAUGUGCCGCAACGCUCUGCCCCUUAAAGCUUCUGCUUUAAGGGGCAUCGUUUAU |
| 240 | Exemplary Nme | mG*mG*mC*CUGGCUGAUGAGGCCGCACAUGUUGUAGCUCCCU*mG*mA*mA*mA*CCGUUGCUACAAUAAGGCCGUmC* |
| | guide; G017564 | mU*mG*mA*mA*mA*mA*mGAUGUGCCGCAACGCUCUGCCG*mC*mU*mU*mC*mU*mG*CGGCAUCGUUU*mA*mU*mC |
| 241 | Exemplary Nme | mG*mG*mC*CUGGCUGAUGAGGCCGCACAUGUUGUAGCUCCCU*mG*mA*mA*mA*CCGUUGCUACAAUAAGGCCGUmC* |
| | guide; G017565 | mU*mG*mA*mA*mA*mA*mGAUGUGCCGCAACGCUCUGCCmU*mU*mC*mUGGCAUCGUUU*mA*mU*mC |
| 242 | Exemplary Nme | mG*mG*mC*CUGGCUGAUGAGGCCGCACAUGUUGUAGCUCCCU*mG*mA*mA*mA*CCGUUGCUACAAUAAGGCCGUmC* |
| | guide; G017566 | mG*mA*mA*mA*mGAUGUGCCGCAACGCUCUGCCmU*mU*mC*mUGGCAUCGUUU*mA*mU*mC |
| 243 | Exemplary Nme | mG*mGC*CUGGCUmGAUmGAGGCCGCACAUmGUUGmUmAmGmCUCCCUmC(L1)mGCCGUUmGmCUAmCAAU*A*AGmGm |
| | guide; G020362 | CCmGmUmC(L1)mGmAmUGUGCmCGCAACGCUCUmGmCC(L1)GGCAUCGUUU*AmU*mU |
| 244 | Exemplary Nme | mG*mGGGCCAGCmUUCmAGACACAAAUACmGUUGmUmAmGmCUCCCmUmGmAmAmAmCmCGUUmGmCUAmCAAU*AAGmG |
| | guide; G021237 | mCCmGmUmCmGmAmAmAmGmAmUGUGCmCGCmAmAmCmGCUCUmGmCCmUmUmCmUGmGCmAmUC*mG*mU*mU |
| 245 | Exemplary Nme | mU*mUUUCACAGmCCAmACGACUCUGGCCmGUUGmUmAmGmCUCCCmUmGmAmAmAmCmCGUUmGmCUAmCAAU*AAGG |
| | guide; G021238 | mCCmGmUmCmGmAmAmAmGmAmUGUGCmCGCmAmAmCmGCUCUmGmCCmUmUmCmUGmGCmAmUC*mG*mU*mU |
| 246 | Exemplary Nme | mC*mUGCCUCGGmACAmGCAUCCAGGACUmGUUGmUmAmGmCUCCCmUmGmAmAmAmCmCGUUmGmCUAmCAAU*AAGG |
| | guide; G021247 | mCCmGmUmCmGmAmAmAmGmAmUGUGCmCGCmAmAmCmGCUCUmGmCCmUmUmCmUGmGCmAmUC*mG*mU*mU |
| 247 | Exemplary Nme | mC*mCAAGUGUCmUUCmCAGUACGAUUUGmGUUGmUmAmGmCUCCCmUmGmAmAmAmCmCGUUmGmCUAmCAAU*AAGmG |
| | guide; G021256 | mCCmGmUmCmGmAmAmAmGmAmUGUGCmCGCmAmAmCmGCUCUmGmCCmUmUmCmUGmGCmAmUC*mG*mU*mU |
| 248 | Exemplary Nme | mU*mCUACAGCAmGGGmCUGCCUCGGACAmGUUGmUmAmGmCUCCCmUmGmAmAmAmCmCGUUmGmCUAmCAAU*AAGmG |
| | guide; G021275 | mCCmGmUmCmGmAmAmAmGmAmUGUGCmCGCmAmAmCmGCUCUmGmCCmUmUmCmUGmGCmAmUC*G*U*mU |
| 249 | Exemplary Nme | mC*mUUCACCAGmGAGmAAGCCGUCACACmGUUGmUmAmGmCUCCCmUmGmAmAmAmCmCGUUmGmCUAmCAAU*AAGmG |
| | guide; G021320 | mCCmGmUmCmGmAmAmAmGmAmUGUGCmCGCmAmAmCmGCUCUmGmCCmUmUmCmUGmGCmAmUC*mG*mU*mU |
| 250 | Exemplary Nme | mC*mU*mUmCmACCmAmGGmAGmAAGCCmGUCAmCACmGUUGmUmAmGmCUCCCmUmGmAmAmAmCmCGUUmGmCUAmCA |
| | guide; G021722 | AU*AAGmGmCCmGmUmCmGmAmAmAmGmAmUGUGCmCGmCAAmCGCUCUmGmCCmUmUmCmUGGCAUCG*mU*mU |
| 251 | Exemplary Nme | mC*mU*mU*mCmACCmAmGGmAGmAAGCCmGUCAmCACmGUUGmUmAmGmCUCCCmUmGmAmAmAmCmCGUUmGmCUAmC |
| | guide; G021845 | AAU*AAGmGmCCmGmUmCmGmAmAmAmGmAmUGUGCmCGmCAAmCGCUCUmGmCCmUmUmCmUGGCAUCG*mU*mU |
| 252 | Exemplary Nme | mC*mU*mU*mCmACCmAmGGmAGmAAGCCmGUCAmCACmGUUGmUmAmGmCUCCCmUmUmC(L1)mGmAmCmCGUUmGmC |
| | guide; G021846 | UAmCAAU*AAGmGmCCmGmUmC(L1)mGmAmUGUGCmCGmCAAmCGCUCUmGmCC(L1)GGCAUCG*mU*mU |
| 253 | Exemplary Nme | mC*mU*mU*mCmACCmAmAGmAGmAAGCCmGUCAmCACmGUUGmUmAmGmCUCCCmUmUmC(L1)mGmAmCmCGUUmGmC |
| | guide; G023420 | UAmCAAU*AAGmGmCCmGmUmC(L1)mGmAmUGUGCmCGmCAAmCGCUCUmGmCC(L1)GGCAUCGmU*mU |
| 254 | Exemplary Spy | CUCCCUGAUGGAGAUGACAGGUUUUAGAGCUAGAAAUAGCAAGUUAAAAUAAGGCUAGUCCGUUAUCAACUUGAAAAAGU |
| | guide; plG000665 | GGCACCGAGUCGGUGCUUUU |
| 255 | Exemplary Spy | CACCUUGGCUGUAGUCAUCUGUUUUAGAGCUAGAAAUAGCAAGUUAAAAUAAGGCUAGUCCGUUAUCAACUUGAAAAAGU |
| | guide; plG012746 | GGCACCGAGUCGGUGCUUUUUUUGGUACCGGUACC |
| 256 | Exemplary Spy | CAGUGCGGUUCCACACAUACGUUUUAGAGCUAGAAAUAGCAAGUUAAAAUAAGGCUAGUCCGUUAUCAACUUGAAAAAGU |
| | guide; plG012684 | GGCACCGAGUCGGUGCUUUUUUUGGUACCGGUACC |
| 257 | Exemplary Spy | AAGCCUGCAUGAGCUCAGUGGUUUUAGAGCUAGAAAUAGCAAGUUAAAAUAAGGCUAGUCCGUUAUCAACUUGAAAAAGU |
| | guide; plG012685 | GGCACCGAGUCGGUGCUUUUUUUGGUACCGGUACC |
| 258 | Exemplary Spy | AUGGGUCACAGACGGGUCCAGUUUUAGAGCUAGAAAUAGCAAGUUAAAAUAAGGCUAGUCCGUUAUCAACUUGAAAAAGU |
| | guide; plG012686 | GGCACCGAGUCGGUGCUUUUUUUGGUACCGGUACC |
| 259 | Exemplary Spy | CUCAAAGAGACCCAUGAGAUGUUUUAGAGCUAGAAAUAGCAAGUUAAAAUAAGGCUAGUCCGUUAUCAACUUGAAAAAGU |
| | guide; plG012687 | GGCACCGAGUCGGUGCUUUUUUUGGUACCGGUACC |
| 260 | Exemplary Spy | AUCUCGUAUUUCAUGUCUCCGUUUUAGAGCUAGAAAUAGCAAGUUAAAAUAAGGCUAGUCCGUUAUCAACUUGAAAAAGU |
| | guide; plG012688 | GGCACCGAGUCGGUGCUUUUUUUGGUACCGGUACC |
| 261 | Target sequence of | UGGCGCUUCGCCAGCCAUUCCUGC |
| | plCR012468 | |
| 262 | Target sequence of | CCCGGGGCGGUCCCGGUCCAAGCG |
| | plCR012469 | |
| 263 | Target sequence of | CUCCCUGAUGGAGAUGACAG |
| | plG000665 | |
| 264 | Target sequence of | CACCUUGGCUGUAGUCAUCU |
| | plG012746 | |
| 265 | Target sequence of | CAGUGCGGUUCCACACAUAC |
| | plG012684 | |
| 266 | Target sequence of | AAGCCUGCAUGAGCUCAGUG |
| | plG012685 | |
| 267 | Target sequence of | AUGGGUCACAGACGGGUCCA |
| | plG012686 | |
| 268 | Target sequence of | CUCAAAGAGACCCAUGAGAU |
| | plG012687 | |
| 269 | Target sequence of | AUCUCGUAUUUCAUGUCUCC |
| | plG012688 | |
| 301 | Exemplary XTEN | SGSETPGTSESATPES |
| 302 | Exemplary XTEN | SGSETPGTSESA |
| 303 | Exemplary XTEN | SGSETPGTSESATPEGGSGGS |
| 304 | amino acid | GGS |
| | sequence for | |
| | exemplary linker | |
| 305 | amino acid | GGGGS |
| | sequence for | |
| | exemplary linker | |
| 306 | amino acid | EAAAK |
| | sequence for | |
| | exemplary linker | |
| 307 | amino acid | SEGSA |
| | sequence for | |
| | exemplary linker | |
| 308 | amino acid | SEGSAGTST |
| | sequence for | |
| | exemplary linker | |
| 309 | amino acid | GGGGSGGGGS |
| | sequence for | |
| | exemplary linker | |
| 310 | amino acid | GGGGSEAAAK |
| | sequence for | |
| | exemplary linker | |
| 311 | amino acid | EAAAKGGGGS |
| | sequence for | |
| | exemplary linker | |
| 312 | amino acid | EAAAKEAAAK |
| | sequence for | |
| | exemplary linker | |
| 313 | amino acid | SEGSAGTSTESEGSA |
| | sequence for | |
| | exemplary linker | |
| 314 | amino acid | GGGGSGGGGSGGGGS |
| | sequence for | |
| | exemplary linker | |
| 315 | amino acid | GGGGSGGGGSEAAAK |
| | sequence for | |
| | exemplary linker | |
| 316 | amino acid | GGGGSEAAAKGGGGS |
| | sequence for | |
| | exemplary linker | |
| 317 | amino acid | EAAAKGGGGSEAAAK |
| | sequence for | |
| | exemplary linker | |
| 318 | amino acid | EAAAKEAAAKGGGGS |
| | sequence for | |
| | exemplary linker | |
| 319 | amino acid | SEGSAGTSTESEGSAGTSTE |
| | sequence for | |
| | exemplary linker | |
| 320 | amino acid | GGGGSGGGGSGGGGSEAAAK |
| | sequence for | |
| | exemplary linker | |
| 321 | amino acid | GGGGSGGGGSEAAAKGGGGS |
| | sequence for | |
| | exemplary linker | |
| 322 | amino acid | GGGGSEAAAKGGGGSEAAAK |
| | sequence for | |
| | exemplary linker | |
| 323 | amino acid | GGGGSEAAAKEAAAKGGGGS |
| | sequence for | |
| | exemplary linker | |
| 324 | amino acid | GGGGSEAAAKEAAAKEAAAK |
| | sequence for | |
| | exemplary linker | |
| 325 | amino acid | EAAAKGGGGSGGGGSGGGGS |
| | sequence for | |
| | exemplary linker | |
| 326 | amino acid | EAAAKGGGGSGGGGSEAAAK |
| | sequence for | |
| | exemplary linker | |
| 327 | amino acid | EAAAKGGGGSEAAAKGGGGS |
| | sequence for | |
| | exemplary linker | |
| 328 | amino acid | EAAAKGGGGSEAAAKEAAAK |
| | sequence for | |
| | exemplary linker | |
| 329 | amino acid | EAAAKEAAAKGGGGSGGGGS |
| | sequence for | |
| | exemplary linker | |
| 330 | amino acid | EAAAKEAAAKGGGGSEAAAK |
| | sequence for | |
| | exemplary linker | |
| 331 | amino acid | EAAAKEAAAKEAAAKGGGGS |
| | sequence for | |
| | exemplary linker | |
| 332 | amino acid | SEGSAGTSTESEGSAGTSTESEGSA |
| | sequence for | |
| | exemplary linker | |
| 333 | amino acid | GGGGSGGGGSGGGGSGGGGSGGGGS |
| | sequence for | |
| | exemplary linker | |
| 334 | amino acid | GGGGSGGGGSGGGGSGGGGSEAAAK |
| | sequence for | |
| | exemplary linker | |
| 335 | amino acid | GGGGSGGGGSGGGGSEAAAKGGGGS |
| | sequence for | |
| | exemplary linker | |
| 336 | amino acid | GGGGSGGGGSGGGGSEAAAKEAAAK |
| | sequence for | |
| | exemplary linker | |
| 337 | amino acid | GGGGSGGGGSEAAAKGGGGSGGGGS |
| | sequence for | |
| | exemplary linker | |
| 338 | amino acid | GGGGSGGGGSEAAAKGGGGSEAAAK |
| | sequence for | |
| | exemplary linker | |
| 339 | amino acid | GGGGSGGGGSEAAAKEAAAKGGGGS |
| | sequence for | |
| | exemplary linker | |
| 340 | amino acid | GGGGSGGGGSEAAAKEAAAKEAAAK |
| | sequence for | |
| | exemplary linker | |
| 341 | amino acid | GGGGSEAAAKGGGGSGGGGSGGGGS |
| | sequence for | |
| | exemplary linker | |
| 342 | amino acid | GGGGSEAAAKGGGGSGGGGSEAAAK |
| | sequence for | |
| | exemplary linker | |
| 343 | amino acid | GGGGSEAAAKGGGGSEAAAKGGGGS |
| | sequence for | |
| | exemplary linker | |
| 344 | amino acid | GGGGSEAAAKGGGGSEAAAKEAAAK |
| | sequence for | |
| | exemplary linker | |
| 345 | amino acid | GGGGSEAAAKEAAAKGGGGSGGGGS |
| | sequence for | |
| | exemplary linker | |
| 346 | amino acid | GGGGSEAAAKEAAAKEAAAKGGGGS |
| | sequence for | |
| | exemplary linker | |
| 347 | amino acid | GGGGSEAAAKEAAAKEAAAKEAAAK |
| | sequence for | |
| | exemplary linker | |
| 348 | amino acid | EAAAKGGGGSGGGGSGGGGSGGGGS |
| | sequence for | |
| | exemplary linker | |
| 349 | amino acid | EAAAKGGGGSGGGGSGGGGSEAAAK |
| | sequence for | |
| | exemplary linker | |
| 350 | amino acid | EAAAKGGGGSGGGGSEAAAKGGGGS |
| | sequence for | |
| | exemplary linker | |
| 351 | amino acid | EAAAKGGGGSGGGGSEAAAKEAAAK |
| | sequence for | |
| | exemplary linker | |
| 352 | amino acid | EAAAKGGGGSEAAAKGGGGSGGGGS |
| | sequence for | |
| | exemplary linker | |
| 353 | amino acid | EAAAKGGGGSEAAAKGGGGSEAAAK |
| | sequence for | |
| | exemplary linker | |
| 354 | amino acid | EAAAKGGGGSEAAAKEAAAKGGGGS |
| | sequence for | |
| | exemplary linker | |
| 355 | amino acid | EAAAKGGGGSEAAAKEAAAKEAAAK |
| | sequence for | |
| | exemplary linker | |
| 356 | amino acid | EAAAKEAAAKGGGGSEAAAKGGGGS |
| | sequence for | |
| | exemplary linker | |
| 357 | amino acid | EAAAKEAAAKGGGGSEAAAKEAAAK |
| | sequence for | |
| | exemplary linker | |
| 358 | amino acid | EAAAKEAAAKEAAAKGGGGSEAAAK |
| | sequence for | |
| | exemplary linker | |
| 359 | amino acid | EAAAKEAAAKEAAAKEAAAKGGGGS |
| | sequence for | |
| | exemplary linker | |
| 360 | amino acid | EAAAKEAAAKEAAAKEAAAKEAAAK |
| | sequence for | |
| | exemplary linker | |
| 361 | amino acid | GTKDSTKDIPETPSKD |
| | sequence for | |
| | exemplary linker | |
| 362 | amino acid | GRDVRQPEVKEEKPES |
| | sequence for | |
| | exemplary linker | |
| 363 | amino acid | EGKSSGSGSESKSTAG |
| | sequence for | |
| | exemplary linker | |
| 364 | amino acid | TPGSPAGSPTSTEEGT |
| | sequence for | |
| | exemplary linker | |
| 365 | amino acid | GSEPATSGSETPGTST |
| | sequence for | |
| | exemplary linker | |
| 366 | SV40 NLS | PKKKRKVE |
| 367 | SV40 NLS | KKKRKVE |
| 368 | bipartite NLS | KRTADGSEFESPKKKRKVE |
| 369 | c-myc like NLS | PAAKKKKLD |
| 370 | Nucleic acid | CCCAAGAAGAAGAGGAAAGTC |
| | sequence for SV40 | |
| | NLS | |
| 371 | Amino acid | PKKKRKV |
| | sequence for SV40 | |
| | NLS | |
| 372 | Exemplary NLS 1 | LAAKRSRTT |
| 373 | Exemplary NLS 2 | QAAKRSRTT |
| 374 | Exemplary NLS 3 | PAPAKRERTT |
| 375 | Exemplary NLS 4 | QAAKRPRTT |
| 376 | Exemplary NLS 5 | RAAKRPRTT |
| 377 | Exemplary NLS 6 | AAAKRSWSMAA |
| 378 | Exemplary NLS 7 | AAAKRVWSMAF |
| 379 | Exemplary NLS 8 | AAAKRSWSMAF |
| 380 | Exemplary NLS 9 | AAAKRKYFAA |
| 381 | Exemplary NLS 10 | RAAKRKAFAA |
| 382 | Exemplary NLS 11 | RAAKRKYFAV |
| 383 | Alternative SV40 | PKKKRRV |
| | NLS | |
| 384 | Nucleoplasmin NLS | KRPAATKKAGQAKKKK |
| 385 | Exemplary coding | CCGAAGAAGAAGAGAAAGGTC |
| | sequence for SV40 | |
| | NLS | |
| 386 | Exemplary coding | CTGGCAGCAAAGAGAAGCAGAACAACA |
| | sequence for NLS1 | |
| 387 | Exemplary coding | CAGGCAGCAAAGAGAAGCAGAACAACA |
| | sequence for NLS2 | |
| 388 | Exemplary coding | CCGGCACCGGCAAAGAGAGAAAGAACAACA |
| | sequence for NLS3 | |
| 389 | Exemplary coding | CAGGCAGCAAAGAGACCGAGAACAACA |
| | sequence for NLS4 | |
| 390 | Exemplary coding | AGAGCAGCAAAGAGACCGAGAACAACA |
| | sequence for NLS5 | |
| 391 | Exemplary coding | GCAGCAGCAAAGAGAAGCTGGAGCATGGCAGCA |
| | sequence for NLS6 | |
| 392 | Exemplary coding | GCAGCAGCAAAGAGAGTCTGGAGCATGGCATTC |
| | sequence for NLS7 | |
| 393 | Exemplary coding | GCAGCAGCAAAGAGAAGCTGGAGCATGGCATTC |
| | sequence for NLS8 | |
| 394 | Exemplary coding | GCAGCAGCAAAGAGAAAGTACTTCGCAGCA |
| | sequence for NLS9 | |
| 395 | Exemplary coding | AGAGCAGCAAAGAGAAAGGCATTCGCAGCA |
| | sequence for NLS10 | |
| 396 | Exemplary coding | AGAGCAGCAAAGAGAAAGTACTTCGCAGTC |
| | sequence for NLS11 | |
| 397 | Exemplary coding | CCGAAGAAGAAGAGAAGAGTC |
| | sequence for | |
| | alternate SV40 NLS | |
| 398 | Exemplary 5′ UTR | ACATTTGCTTCTGACACAACTGTGTTCACTAGCAACCTCAAACAGACACC |
| 399 | Exemplary 5′ UTR | CATAAACCCTGGCGCGCTCGCGGCCCGGCACTCTTCTGGTCCCCACAGACTCAGAGAGAACCCACC |
| 400 | Exemplary 5′ UTR | AAGCTCAGAATAAACGCTCAACTTTGGCC |
| 401 | Exemplary 5′ UTR | CAGGGTCCTGTGGACAGCTCACCAGCT |
| 402 | Exemplary 5′ UTR | TCCCGCAGTCGGCGTCCAGCGGCTCTGCTTGTTCGTGTGTGTGTCGTTGCAGGCCTTATTC |
| 403 | Exemplary 5′ UTR | CAGATCGCCTGGAGACGCCATCCACGCTGTTTTGACCTCCAT |
| 404 | Exemplary 5′ UTR | AGAAGACACCGGGACCGATCCAGCCTCCGCGGCCGGGAACGG |
| 405 | Exemplary 5′ UTR | TGCATTGGAACGCGGATTCCCCGTGCCAAGAGTGACTCACCG |
| 406 | Exemplary 3′ UTR | GCTCGCTTTCTTGCTGTCCAATTTCTATTAAAGGTTCCTTTGTTCCCTAAGTCCAACTACTAAACTGGGGGATATTATGA |
| | | AGGGCCTTGAGCATCTGGATTCTGCCTAATAAAAAACATTTATTTTCATTGC |
| 407 | Exemplary 3′ UTR | GCTGGAGCCTCGGTGGCCATGCTTCTTGCCCCTTGGGCCTCCCCCCAGCCCCTCCTCCCCTTCCTGCACCCGTACCCCCG |
| | | TGGTCTTTGAATAAAGTCTGAGTGGGCGGC |
| 408 | Exemplary 3′ UTR | ACCAGCCTCAAGAACACCCGAATGGAGTCTCTAAGCTACATAATACCAACTTACACTTTACAAAATGTTGTCCCCCAAAA |
| | | TGTAGCCATTCGTATCTGCTCCTAATAAAAAGAAAGTTTCTTCACATTCT |
| 409 | Exemplary 3′ UTR | TTGCCAGCCATCTGTTGTTTGCCCCTCCCCCGTGCCTTCCTTGACCCTGGAAGGTGCCACTCCCACTGTCCTTTCCTAAT |
| | | AAAATGAGGAAATTGCATCGCA |
| 410 | Exemplary 3′ UTR | GCTGCCTTCTGCGGGGCTTGCCTTCTGGCCATGCCCTTCTTCTCTCCCTTGCACCTGTACCTCTTGGTCTTTGAATAAAG |
| | | CCTGAGTAGGAAG |
| 411 | Exemplary 3′ UTR | CTGGTACTGCATGCACGCAATGCTAGCTGCCCCTTTCCCGTCCTGGGTACCCCGAGTCTCCCCCGACCTCGGGTCCCAGG |
| | | TATGCTCCCACCTCCACCTGCCCCACTCACCACCTCTGCTAGTTCCAGACACCTCCCAAGCACGCAGCAATGCAGCTCAA |
| | | AACGCTTAGCCTAGCCACACCCCCACGGGAAACAGCAGTGATTAACCTTTAGCAATAAACGAAAGTTTAACTAAGCTATA |
| | | CTAACCCCAGGGTTGGTCAATTTCGTGCCAGCCACACC |
| 412 | Exemplary 3′ UTR | CAAGCACGCAGCAATGCAGCTCAAAACGCTTAGCCTAGCCACACCCCCACGGGAAACAGCAGTGATTAACCTTTAGCAAT |
| | | AAACGAAAGTTTAACTAAGCTATACTAACCCCAGGGTTGGTCAATTTCGTGCCAGCCACACCCTGGTACTGCATGCACGC |
| | | AATGCTAGCTGCCCCTTTCCCGTCCTGGGTACCCCGAGTCTCCCCCGACCTCGGGTCCCAGGTATGCTCCCACCTCCACC |
| | | TGCCCCACTCACCACCTCTGCTAGTTCCAGACACCTCC |
| 413 | Exemplary 3′ UTR | ACCAGCCTCAAGAACACCCGAATGGAGTCTCTAAGCTACATAATACCAACTTACACTTTACAAAATGTTGTCCCCCAAAA |
| | | TGTAGCCATTCGTATCTGCTCCTAATAAAAAGAAAGTTTCTTCACATTCTACCAGCCTCAAGAACACCCGAATGGAGTCT |
| | | CTAAGCTACATAATACCAACTTACACTTTACAAAATGTTGTCCCCCAAAATGTAGCCATTCGTATCTGCTCCTAATAAAA |
| | | AGAAAGTTTCTTCACATTCT |
| 414 | Exemplary Kozak | GCCRCCAUGG |
| | sequence | |
| 415 | Exemplary Kozak | GCCGCCRCCAUGG |
| | sequence | |
| 416 | Exemplary poly-A | AAAAAAAAAAAAAAAAAAAAAAAAAAAAAAGCGAAAAAAAAAAAAAAAAAAAAAAAAAAAAAACCGAAAAAAAAAAAAAA |
| | sequence | AAAAAAAAAAAAAAAAAAAAAAAAA |

TABLE 39
Exemplary SpyCas9 sgRNA conserved portion (“Exemplary SpyCas9 sgRNA-1”; SEQ ID NO: 161)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 40
Exemplary NmeCas9 sgRNA (SEQ ID NO: 279 (“Exemplary NmeCas9 sgRNA-1)

| | | | | | | | | | | | | | | | | | | | | | | | | |
|---|---|---|---|---|---|---|---|---|---|---|---|---|---|---|---|---|---|---|---|---|---|---|---|---|
| 1-24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 |
| NNNNNNNNNNNNNNNNNNNNNNNN | G | U | U | G | U | A | G | C | U | C | C | C | U | U | U | C | U | C | A | U | U | U | C | G |

| | | | | | | | | | | | Lower stem | | | | | | | | | Upper stem | | | | | | | | |
|---|---|---|---|---|---|---|---|---|---|---|---|---|---|---|---|---|---|---|---|---|---|---|---|---|---|---|---|---|
| Guide region | | | | | | | | | | | Repeat/Anti-Repeat region | | | | | | | | | | | | | | | | | |
| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 | 77 |
| G | A | A | A | C | G | A | A | A | U | G | A | G | A | A | C | C | G | U | U | G | C | U | A | C | A | A | U | A |

| | | |
|---|---|---|
| Loop | Upper stem | Lower Stem |

Repeat/Anti-Repeat region

| | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|
| 78 | 79 | 80 | 81 | 82 | 83 | 84 | 85 | 86 | 87 | 88 | 89 | 90 | 91 | 92 | 93 | 94 | 95 | 96 | 97 | 98 | 99 | 100 | 101 | 102 | 103 | 104 | 105 | 106 | 107 | 108 |
| A | G | G | C | C | G | U | C | U | G | A | A | A | A | G | A | U | G | U | G | C | C | G | C | A | A | C | G | C | U | C |

| | | | | |
|---|---|---|---|---|
| Stem | Loop | Stem (96: unpaired) | Lower stem | Bulge |

| | |
|---|---|
| Hairpin 1 | Hairpin 2 |

| | | | | | | | | | | | | | | | | | | | | | | | | | |
|---|---|---|---|---|---|---|---|---|---|---|---|---|---|---|---|---|---|---|---|---|---|---|---|---|---|
| 109 | 110 | 111 | 112 | 113 | 114 | 115 | 116 | 117 | 118 | 119 | 120 | 121 | 122 | 123 | 124 | 125 | 126 | 127 | 128 | 129 | 130 | 131 | 132 | 133 | 134 |
| U | G | C | C | C | C | U | U | A | A | A | G | C | U | U | C | U | G | C | U | U | U | A | A | G | G |

| | | |
|---|---|---|
| Upper Stem | Loop | Upper Stem |

Hairpin 2

| | | | | | | | | | | |
|---|---|---|---|---|---|---|---|---|---|---|
| 135 | 136 | 137 | 138 | 139 | 140 | 141 | 142 | 143 | 144 | 145 |
| G | G | C | A | U | C | G | U | U | U | A |

| | | |
|---|---|---|
| Upper Stem | Bulge | Lower Stem |

| | |
|---|---|
| Hairpin 2 | Tail |

We claim:

1. A method of producing a modification in the genome of a target cell, the method comprising contacting the cell with:
(a) a fusion protein, or a nucleic acid encoding a fusion protein, wherein the fusion protein comprises a first cleavase and a second cleavase, wherein:
a. the first cleavase is a Streptococcus pyogenes (Spy)Cas9 cleavase, said SpyCas9 cleavase comprising a R1333K mutation within its protospacer adjacent motif recognition domain; and
b. the second cleavase is a Neisseria meningitidis (Nme)Cas9 cleavase, a Campylobacter jejuni (Cje) Cas9 cleavase, or a Simonsiella muelleri (Smu) Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

2. A method of producing a cell or a population of cells comprising a modification in the genome of the target cell or cells, the method comprising contacting the cell or cells with:
(a) a fusion protein, or a nucleic acid encoding a fusion protein, wherein the fusion protein comprises a first cleavase and a second cleavase, wherein:
a. the first cleavase is a S. pyogenes (Spy)Cas9 cleavase, said SpyCas9 cleavase comprising a R1333K mutation within its protospacer adjacent motif recognition domain; and
b. the second cleavase is a N. meningitidis (Nme)Cas9 cleavase, a C. jejuni (Cje)Cas9 cleavase, or a S. muelleri (Smu)Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

3. The method of any one of claim 1 or 2, wherein the first cleavase is located N-terminal to the second cleavase.

4. The method of any one of claim 1 or 2, wherein the first cleavase is located C-terminal to the second cleavase.

5. The method of any one of claims 1-4, wherein the first guide RNA and the second guide RNA target two non-overlapping genomic loci, optionally wherein the two non-overlapping genomic loci are separated by equal to or less than 150, 140, 130, 120, 110, 100, 90, 80, 70, 60, 50, 40, 30, or 25 nucleotides.

6. The method of claim 5, wherein the two non-overlapping genomic loci are separated by equal to or less than 110 nucleotides.

7. The method of any one of the preceding claims, wherein the first guide RNA is a single guide RNA (sgRNA), optionally a SpyCas9 guide RNA.

8. The method of claim 7, wherein the SpyCas9 guide RNA is a single guide RNA comprising:
a conserved portion of an sgRNA comprising an upper stem and hairpin region, wherein every nucleotide in the upper stem region is modified with 2′-O-Me, and every nucleotide in the hairpin region is modified with 2′-O-Me;
a 3′ end modification comprising 2′-O-Me modified nucleotides at the last three nucleotides of the 3′ end and phosphorothioate (PS) bonds between the last four nucleotides of the 3′ end; and
5′ end modification comprising 2′-O-Me modified nucleotides at the first three nucleotides of the 5′ end; and phosphorothioate (PS) bonds between the first four nucleotides of the 5′ end.

9. The method of claim 7 or 8, wherein the SpyCas9 guide RNA is a short-single guide RNA (short-sgRNA) comprising a conserved portion of an sgRNA comprising a hairpin region, wherein the hairpin region lacks at least 5-10 nucleotides and wherein the short-sgRNA comprises (i) a 5′ end modification or (ii) a 3′ end modification, optionally comprising a nucleotide sequence selected from SEQ ID NOs: 159-167, 170-177, and 180-194, or a nucleotide sequence that is at least 85%, 90%, or 95% identical to SEQ ID NOs: 159-167, 170-177, and 180-194.

10. The method of any one of claims 1-9, wherein the second guide RNA is a single guide RNA (sgRNA), optionally a NmeCas9 guide RNA.

11. The method of claim 10, wherein the second guide RNA is a shortened or chemically modified single guide RNA (sgRNA).

12. The method of any one of claims 1-11, wherein the second guide RNA is a NmeCas9 guide RNA that is a single guide RNA comprising a nucleotide sequence selected from SEQ ID NOs: 280-297, or a nucleotide sequence that is at least 85%, 90%, or 95% identical to SEQ ID NOs: 280-297.

13. The method of claim 12, wherein the second guide RNA comprises one or more internal polyethylene glycol (PEG) linker, optionally wherein the second guide RNA comprises at least 85%, 90%, 95%, 99%, 100% identical to a sequence selected from SEQ ID NOs: 272-278.

14. The method of any one of claims 1-13, wherein one or both of the guide RNAs comprises one or more mismatches to the target sequences.

15. The method of any one of claims 1-14, wherein the nucleic acid encoding the fusion protein is delivered to the cell on at least one vector.

16. The method of any one of claims 1-15, wherein one or more of the fusion protein or the nucleic acid encoding the fusion protein, the first guide RNA, and the second guide RNA are delivered to the cell via electroporation.

17. The method of any one of claims 1-16, wherein the modification is in vivo.

18. The method of any one of claims 1-17, wherein the modification is ex vivo.

19. The method of any one of claims 1-18, wherein the modification comprises a deletion of equal to or less than 150, 140, 130, 120, 110, 100, 90, 80, 70, 60, 50, 40, 30, or 25 nucleotides, optionally wherein the modification comprises a deletion of equal to or less than 150, 140, 130, 120, 110, 100, 90, 80, 70, 60, 50, 40, 30, or 25 contiguous nucleotides.

20. The method of any one of claims 1-19, wherein the modification comprises a deletion of equal to or larger than 25, 35, 45, 55, 65, 75, 85, 95, 100, 105 nucleotides, optionally wherein the modification comprises a deletion of equal to or larger than 25, 35, 45, 55, 65, 75, 85, 95, 100, 105 contiguous nucleotides.

21. The method of any one of claims 1-20, wherein the modification comprises a deletion of each of the nucleotides between a first cleavage site and a second cleavage site.

22. The method of any one of claims 19-21, wherein the deletion comprises one or both protospacer adjacent motif (PAM) sites recognized by the first cleavase or the second cleavase.

23. The method of any one of claims 1-22, wherein the modification increases the expression of one or more RNAs or proteins, optionally wherein the modification increases the expression of the one or more RNAs or proteins by at least two-fold.

24. The method of any one of claims 1-23, wherein the modification results in the deletion of a start codon.

25. The method of any one of claims 1-24, wherein the modification reduces or eliminates the expression of one or more mRNAs or proteins, optionally wherein the modification reduces or eliminates the expression of one or more mRNAs or proteins by at least 50%, at least 55%, at least 60%, at least 65%, at least 70%, at least 75%, at least 80%, at least 85%, at least 90%, or at least 95%.

26. The method of any one of claims 1-25, wherein the cell is in a subject.

27. The method of any one of claims 1-26, wherein the cell comprises
a. a kidney cell;
b. a liver cell;
c. a cell selected from a mesenchymal stem cell, a hematopoietic stem cell (HSC), a mononuclear cell, an endothelial progenitor cells (EPC), a neural stem cells (NSC), a limbal stem cell (LSC), a tissue-specific primary cell or a cell derived therefrom (TSC), an induced pluripotent stem cell (iPSC), an ocular stem cell, a pluripotent stem cell (PSC), an embryonic stem cell (ESC), and a cell for organ or tissue transplantation;
d. an immune cell,
e. a T-cell; or
f. a lymphocyte.

28. An engineered cell or population of engineered cells altered by the method of any one of claims 1-27.

29. The engineered cell or population of engineered cells of claim 28, wherein the genetic modification comprises a deletion of equal to or less than 110, 100, 90, 80, 70, 60, 50, 40, 30, or 25 nucleotides, optionally wherein the deletion comprises one or both protospacer adjacent motif (PAM) sites.

30. A polynucleotide comprising an open reading frame (ORF) encoding a fusion protein, wherein the fusion protein comprises a first cleavase and a second cleavase, wherein:
a. the first cleavase is a S. pyogenes (Spy)Cas9 cleavase, said SpyCas9 cleavase comprising a R1333K mutation within its protospacer adjacent motif recognition domain; and
b. the second cleavase is a N. meningitidis (Nme)Cas9 cleavase, a C. jejuni (Cje) Cas9 cleavase, or a S. muelleri (Smu) Cas9 cleavase.

31. A composition comprising
(a) a polynucleotide comprising an open reading frame (ORF) encoding a fusion protein, wherein the fusion protein comprises a first cleavase and a second cleavase, wherein:
a. the first cleavase is a S. pyogenes (Spy)Cas9 cleavase, said SpyCas9 cleavase comprising a R1333K mutation within its protospacer adjacent motif recognition domain; and
b. the second cleavase is a N. meningitidis (Nme)Cas9 cleavase, a C. jejuni (Cje) Cas9 cleavase, or a Simonsiella muelleri (Smu) Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

32. One or more lipid nanoparticles comprising:
(a) a polynucleotide comprising an open reading frame (ORF) encoding a fusion protein, wherein the fusion protein comprises a first cleavase and a second cleavase, wherein:
a. the first cleavase is a S. pyogenes (Spy)Cas9 cleavase, said SpyCas9 cleavase comprising a R1333K mutation within its protospacer adjacent motif recognition domain; and
b. the second cleavase is a N. meningitidis (Nme)Cas9 cleavase, a C. jejuni (Cje) Cas9 cleavase, or a Simonsiella muelleri (Smu) Cas9 cleavase; and
(b) a first guide RNA that directs the first cleavase to a first genomic locus; and
(c) a second guide RNA that directs the second cleavase to a second genomic locus,
wherein the second genomic locus is different from the first genomic locus.

33. The method, polynucleotide, composition or lipid nanoparticles of any one of claims 1-32, wherein (i) the SpyCas9 cleavase comprises an amino acid sequence of SEQ ID NO: 105 or an amino acid sequence that is at least 85%, at least 90%, or at least 95% identical to SEQ ID NO: 105; or (ii) the nucleotide encoding the SpyCas9 cleavase comprises an open reading frame (ORF) comprising a sequence of SEQ ID NO: 104 or a nucleotide sequence that is at least 85, at least 90%, or at least 95% identical to SEQ ID NO: 104.

34. The method, polynucleotide, composition or lipid nanoparticles of any one of claims 1-33, wherein the second cleavase is a NmeCas9 cleavase.

35. The method, polynucleotide, composition or lipid nanoparticles of any one of claims 1-34, wherein the NmeCas9 cleavase is an Nme1Cas9, an Nme2Cas9, or an Nme3Cas9.

36. The method, polynucleotide, composition or lipid nanoparticles of any one of claims 1-35, wherein (i) the NmeCas9 cleavase comprises an amino acid sequence of any one of SEQ ID NO: 22, 107, 109, 120, 127, 136, or 137 or an amino acid sequence that is at least 85%, at least 90%, at least 95% identical to any one of SEQ ID NO: 22, 107, 109, 120, 127, 136, or 137; or (ii) the nucleotide encoding the NmeCas9 cleavase comprises a nucleotide sequence of any one of SEQ ID NO: 21, 106, 108, 121-126, 128-133, 134, 135, 138, or 139; or a nucleotide sequence that is at least 85%, at least 90%, or at least 95% identical to any one of SEQ ID NO: SEQ ID NO: 21, 106, 108, 121-126, 128-133, 134, 135, 138, or 139.

37. The method, polynucleotide, composition or lipid nanoparticles of any one of claims 1-36, wherein (a) the NmeCas9 cleavase is a Nme2Cas9 comprises an amino acid sequence of any one of SEQ ID NO: 22, 109, or 136, or an amino acid sequence that is at least 85%, at least 90%, at least 95% identical to any one of SEQ ID NO: 22, 109, or 136; or (b) the nucleotide encoding the NmeCas9 cleavase comprises a nucleotide sequence of any one of SEQ ID NO: 21, 108, or 138; or a nucleotide sequence that is at least 85%, at least 90%, or at least 95% identical to any one of SEQ ID NO: SEQ ID NO: 21, 108, or 138.

38. The method, polynucleotide, composition or lipid nanoparticles of any one of claims 1-37, wherein (a) the CjeCas9 cleavase comprises an amino acid sequence of SEQ ID NO: 144; or an amino acid sequence that is at least 85%, at least 90%, or at least 95% identical to SEQ ID NO: 144; or (b) the nucleotide encoding the CjeCas9 cleavase comprises a sequence of SEQ ID NO: 143 or a nucleotide sequence that is at least 85%, at least 90%, or at least 95% identical to SEQ ID NO: 143.

39. The method polynucleotide, composition or lipid nanoparticles of any one of claims 1-38, wherein (a) the SmuCas9 cleavase comprises an amino acid sequence of SEQ ID NO: 142; or an amino acid sequence that is at least 85%, at least 90%, or at least 95% identical to SEQ ID NO: 142; or (b) the nucleotide encoding the SmuCas9 cleavase comprises an open reading frame (ORF) comprising a sequence of SEQ ID NO: 140 or 141 or a nucleotide sequence that is at least 85%, at least 90%, or at least 95% identical to SEQ ID NO: 140 or 141.

40. The method, polynucleotide, composition or lipid nanoparticles of any one of claims 1-39, wherein the fusion protein comprises a peptide linker between the first cleavase and the second cleavase, optionally wherein the peptide linker comprises
a. at least 10, at least 20, at least 30, at least 40, at least 50, at least 60, at least 70, at least 80 amino acid residues; or
b. 11, 21, 31, 41, 51, 61, 71, or 81 amino acid residues.

41. The method, polynucleotide, composition or lipid nanoparticles of any one of claims 1-40, wherein the fusion protein comprises a peptide linker between the first cleavase and the second cleavase and the peptide linker comprises an amino acid sequence of any one of SEQ ID NOs: 150-158; or an amino acid sequence is at least 85%, at least 90%, or at least 95% identical to any one of SEQ ID NOs: 150-158.

42. The method, polynucleotide, composition or lipid nanoparticles of any one of claims 1-41, wherein the fusion protein comprises a nuclear localization signal (NLS), optionally wherein
a. the NLS is present at the C-terminus of the fusion protein;
b. the NLS is present at the N-terminus of the fusion protein; or
c. the NLS is present at both the N-terminus and C-terminus of the fusion protein.

43. The method, polynucleotide, composition or lipid nanoparticles of any one of any one of claims 1-42, wherein the fusion protein comprises a nuclear localization signal (NLS), and wherein the NLS comprises a sequence having at least 80%, at least 85%, at least 90%, or at least 95% identity to any one of SEQ ID NOs: 366-369 and 371-384 or is encoded by a sequence having at least 80%, at least 85%, at least 90%, at least 95%, at least 98% or 100% identity to the sequence of any one of SEQ ID NOs: 370 and 385-397.

44. The method, polynucleotide, composition or lipid nanoparticles of any one of claims 1-43, wherein the fusion protein comprise one, two, or three nuclear localization signals (NLSs) independently selected from SEQ ID NOs: 366-369 and 371-384.

45. The method, polynucleotide, composition or lipid nanoparticles of any one of claims 1-44, wherein
a. the fusion protein comprises, from N-terminus to C-terminus:
i. the first cleavase;
ii. a peptide linker, optionally wherein the linker comprises 81 amino acid residues;
iii. the second cleavase; and
iv. an NLS comprising an SV40 NLS;
b. the fusion protein comprises, from N-terminus to C-terminus:
i. a first NLS, wherein the first NLS comprises an SV40 NLS;
ii. the second cleavase;
iii. a peptide linker, optionally wherein the peptide linker comprises 41 amino acids;
iv. the first cleavase;
v. a second NLS, wherein the second NLS comprising an SV40 NLS; or
c. the fusion protein comprises, from N-terminus to C-terminus:
i. the second cleavase;
ii. a peptide linker, optionally wherein the peptide linker comprises 41 amino acids;
iii. the first cleavase; and
iv. an NLS, optionally wherein the NLS comprises an SV40 NLS.

46. The method, polynucleotide, composition or lipid nanoparticles of any one of claims 1-45, wherein
(a) the fusion protein comprise an amino acid sequence of SEQ ID NOs: 3, 5, 7, 10, 13, 16, 40, 43, 45, 47, 50, 52, 55, 57, 60, 62, 65, 67, 70, 72, 75, 77, 80, 82, 85, 87, 90, 92, 101, or 105, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, 13, 16, 40, 43, 45, 47, 50, 52, 55, 57, 60, 62, 65, 67, 70, 72, 75, 77, 80, 82, 85, 87, 90, 92, 101, or 105; or
(b) the nucleic acid encoding the fusion protein comprises a nucleotide sequence of SEQ ID NOs: 1-2, 4, 6-8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11, 12, 14-15, 38-39, 41-42, 44, 46, 48-49, 51, 53, 54, 56, 58, 59, 61, 63, 64, 66, 68, 69, 71, 73, 74, 76, 78, 79, 81, 83, 84, 86, 88, 89, 91, 100, or 104.

47. The method, polynucleotide, composition or lipid nanoparticles of any one of claims 1-46, wherein
(a) the fusion protein comprise an amino acid sequence of SEQ ID NOs: 3, 5, 7, 10, or 13, or an amino acid sequence that is at least 90%, or at least 95% identical to SEQ ID NO: 3, or an amino acid sequence that is at least 85%, at least 90%, or at least 95%% identical to an amino acid sequence of SEQ ID NOs: 5, 7, 10, or 13; or
(b) the nucleic acid encoding the fusion protein comprises a nucleotide sequence of SEQ ID NOs: 1, 2, 4, 6, 8, 9, 11 or 12, or a nucleotide sequence that is at least 90%, or at least 95% identical to SEQ ID NO: 1 or 2, or a nucleotide sequence that is at least 85%, at least 90%, or at least 95% identical to SEQ ID NO: 4, 6, 8, 9, 11 or 12.

48. The method, polynucleotide, composition, or lipid nanoparticles of any one of claims 1-47, wherein
(a) the fusion protein comprise an amino acid sequence of SEQ ID NOs: 5, 7, 10, 13, or 99, or an amino acid sequence that is at least 85%, at least 90%, or at least 95%% identical to an amino acid sequence of SEQ ID NOs: 5, 7, 10, 13, or 99; or
(b) the nucleic acid encoding the fusion protein comprises a nucleotide sequence of SEQ ID NOs: 4, 6, 8, 9, 11 or 12, a nucleotide sequence that is at least 85%, at least 90%, or at least 95% identical to SEQ ID NO: 4, 6, 8, 9, 11 or 12,

49. The method, polynucleotide, composition, or lipid nanoparticles of claim 48, wherein
a. the first polypeptide comprise an amino acid sequence of SEQ ID NOs: 28 or 31 or an amino acid sequence that is at least 85%, at least 90%, or at least 95% identical to SEQ ID NO: 28 or 31; or the nucleic acid or nucleic acids encoding the polypeptide or polypeptides comprises a sequence of SEQ ID NOs: 27 or 30, or a nucleotide sequence that is at least 85%, at least 90%, or at least 95% identical to SEQ ID NOs: 27 or 30; or
b. the second polypeptide comprise an amino acid sequence of SEQ ID NOs: 25 or 34 or an amino acid sequence that is at least 85%, at least 90%, or at least 95% identical to SEQ ID NO: 25 or 34; or the nucleic acid or nucleic acids encoding the polypeptide or polypeptides comprises a sequence of SEQ ID NOs: 24 or 33, or a nucleotide sequence that is at least 85%, at least 90%, or at least 95% identical to SEQ ID NOs: 24 or 33.

50. The polynucleotide, composition or lipid nanoparticles of any one of claims 30-49, wherein the polynucleotide comprises
a. a 5′ UTR with at least 85%, at least 90%, or at least 95% identity to any one of SEQ ID NOs: 398-405;
b. a 3′ UTR with at least 85%, at least 90%, or at least 95% identity to any one of SEQ ID NOs: 406-413; or
c. a 5′ cap, optionally wherein the 5′ cap is Cap0, Cap1, or Cap2.

51. The polynucleotide, composition or lipid nanoparticles of any one of claims 30-50, wherein the polynucleotide is an mRNA.

52. The polynucleotide, composition or lipid nanoparticles of any one of claims 30-51, wherein at least 85% of the uridine is substituted with a modified uridine.

53. The method, composition, or lipid nanoparticles of any one of claims 1-52, wherein one or more of the nucleic acids encoding the fusion protein, the first guide RNA, and the second guide RNA are associated with one or more lipid nanoparticle (LNP).

54. The method, composition, or lipid nanoparticles of any one of claims 1-53, wherein
a. the nucleic acids encoding the fusion protein are each associated with a separate lipid nanoparticle (LNP);
b. the first guide RNA and the second guide RNA are associated with a same lipid nanoparticle (LNP); or
c. all of the nucleic acids encoding the fusion protein, the first guide RNA, and the second guide RNA are associated with a same lipid nanoparticle.

55. The method, composition or lipid nanoparticles of any one of claims 53-54, wherein the LNP comprises (i) an ionizable lipid; (ii) a helper lipid; (iii) a stealth lipid; (iv) a neutral lipid; or combinations of one or more of (i)-(iv), optionally wherein:
a. the ionizable lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate;
b. the helper lipid is cholesterol;
c. the stealth lipid is PEG-DMG; or
d. the neutral lipid is DSPC.

56. The method, composition, or lipid nanoparticles of any one of claims 53-55, wherein the PEG-DMG is 1,2-dimyristoyl-rac-glycero-3-methoxypolyethylene glycol-2000 (PEG2k-DMG).

57. The method, composition, or lipid nanoparticles of any one of claims 53-56, wherein the LNP composition comprises about 50 mol-% ionizable lipid; about 9 mol-% neutral lipid; about 3 mol-% of stealth lipid, and the remainder of the lipid component is helper lipid such as cholesterol.

58. The method, composition, or lipid nanoparticles of any one of claims 53-57, wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

59. A polypeptide encoded by the polynucleotide of any one of claims 30-52.

60. A vector comprising a sequence encoding the polynucleotide of any one of claims 30-52 or an expression construct comprising a promoter operably linked to a sequence encoding the polynucleotide of any one of claims 30-52, optionally wherein the expression construct is in a plasmid.

61. A host cell comprising the vector or expression construct of claim 60.

62. A pharmaceutical composition comprising the polynucleotide, composition, lipid nanoparticle, or polypeptide of any one of claims 30-59, and a pharmaceutically acceptable carrier.

63. A kit comprising the polynucleotide, composition, or polypeptide of any one of claims 30-59.

64. Use of the polynucleotide, composition, lipid nanoparticle or polypeptide any one of claims 30-59 for producing a modification in the genome of a target cell.

65. Use of the polynucleotide, composition, lipid nanoparticle or polypeptide any one of claims 30-59 for the manufacture of a medicament for producing a modification in the genome of a target cell.

66. The method or composition of any one of claims 1-27, 31, and 33-58, wherein one or more of the nucleic acids encoding the fusion protein, the first guide RNA, and the second guide RNA are associated with one or more targeted LNP.

67. The method or composition of claim 66, wherein the targeted LNP is targeted to one or more of the brain, eye, muscle, liver, lung, spleen, and bone marrow.

68. The method or composition of any one of claims 66-67, wherein the targeted LNP comprises a targeting lipid component or a targeting domain.

69. The method or composition of claim 68, wherein the targeting domain comprises a nucleic acid, peptide, antibody, small molecule, glycan, sugar, or hormone.

70. The method of any one of claims 66-69, wherein the targeted LNP is administered by a delivery route of intravenous, intradermal, subcutaneous, inhalation, intranasal, or intramuscular delivery.
